(19) United States
(12) Patent Application Publication
(10) Pub. No.: US 2025/0161481 A1
(43) Pub. Date: May 22, 2025

(54) TARGETED LNP DELIVERY

(71) Applicant: ModeX Therapeutics, Inc., Natick, MA (US)

(72) Inventors: Yen-Chun LU, Somerville, MA (US); Timothy Kosarick EITAS, Mansfield, MA (US); Nicholas Edward JONES, Northborough, MA (US); Leon BROWN, Spencer, MA (US); Hao CHEN, Acton, MA (US); Md Munan SHAIK, Arlington, MA (US); Ronnie R. WEI, Western, MA (US); John R. MASCOLA, Rockville, MD (US); Elias A. ZERHOUNI, Fort Lauderdale, FL (US); Gary J. NABEL, Delray Beach, FL (US); Zhi-yong YANG, Newton Center, MA (US); Lan WU, Chestnut Hill, MA (US); Sian LIAO, Billerica, MA (US); Elissa Kathleen LEONARD, Waltham, MA (US); Mohui WEI, Boston, MA (US)

(21) Appl. No.: 18/933,688

(22) Filed: Oct. 31, 2024

Related U.S. Application Data
(60) Provisional application No. 63/594,875, filed on Oct. 31, 2023.
(60) Provisional application No. 63/672,190, filed on Jul. 16, 2024.

(51) Int. Cl. A61K 47/69; A61K 38/17; A61K 47/68; C07C 219/06; C07C 229/22

(52) U.S. Cl. CPC A61K 47/6925; A61K 38/1774; A61K 47/6849; C07C 219/06; C07C 229/22

(57) ABSTRACT

Disclosed are lipid nanoparticle (LNP) delivery systems that specifically target T cells. The LNP delivery system comprises antibodies conjugated to the surface of the LNP, e.g., via maleimide chemistry, that target at least two T cell surface proteins, e.g., CD3 and CD28. The LNP delivery system can have a single population of LNP conjugated to either a bispecific antiCD3/antiCD28 antibody, or two monospecific antiCD3 and antiCD28 antibodies, or two populations of LNP wherein each population comprises a monospecific antibody. The payload of the LNP delivery system can be, e.g., mRNA encoding chimeric antigen receptors (CAR), a linear DNA fragment or a plasmid encoding chimeric antigen receptors (CAR) or therapeutic proteins such as antibodies, components of a gene editing systems (e.g., CRISPR-Cas), small molecules, antibody-drug conjugates (ADC), and any combination thereof, either encapsulated in the LNP or attached to its surface (e.g., conjugated). Also provided are lipids, pharmaceutical compositions, kits, and methods of treatment.

### CROSS-REFERENCE TO RELATED APPLICATIONS AND INCORPORATION BY REFERENCE

This application claims the priority benefit of U.S. Provisional Applications Nos. 63/594,875, filed Oct. 31, 2023, and 63/672,190, filed Jul. 16, 2024, which are herein incorporated by reference in their entireties.

### REFERENCE TO SEQUENCE LISTING SUBMITTED ELECTRONICALLY

The content of the electronically submitted ST.26 listing in XML format (Name 4850_018PC02_SequenceListing_ST26.xml; Size: 831,516 bytes, and Date of Creation: Oct. 24, 2024) filed with the application is incorporated herein by reference in its entirety.

### FIELD

The present disclosure relates to lipids and targeted delivery of therapeutic agents to specific tissues or cells, e.g., T cells, using engineered lipid nanoparticles.

### BACKGROUND

Delivery of mRNA via Lipid Nanoparticles (LNP) has proven to be an effective and robust way to deliver genes expressing therapeutic proteins, antibodies and vaccines into humans for therapeutic purposes. mRNA/LNP technology has become a fast-growing field in biotech industry after the proof-of-concept demonstration in the development of the mRNA COVID-19 vaccines. The technology has enormous upside with advantages in fast research development, uniform manufacturing process and novel process of delivering biotherapeutics and vaccines, albeit overcoming technical hurdles through R & D investment.

One of the expanding applications of LNP technology is to deliver mRNA or DNA cargos to specific cell type in vivo, including human primary T cells. Gene-based in vivo delivery to T cells can provide the possibilities to augment T cell functions in vivo for therapeutic applications. Current LNP systems have been manufactured in such a way that they increase the likelihood of uptake of the LNP into several cell types, but frequently lack the specificity.

### BRIEF SUMMARY

A targeted delivery system comprising (a) an LNP or combination thereof comprising a first antibody or antigen-binding portion thereof anchored to the surface of the LNP and a payload, wherein: (i) the first antibody or antigen-binding portion thereof specifically binds to a first target molecule on the surface of a T cell or B-cell, and (ii) the binding of the first antibody or antigen-binding portion thereof to the first target molecule induces T cell or B-cell uptake of the payload, and (b) a costimulator that specifically binds to a second target molecule on the surface of the T cell or B-cell, wherein (i) the binding of the costimulator to the second target molecule increases expression of the payload, and (ii) the costimulator is selected from the group consisting of: a second specificity of the first antibody, wherein the second specificity targets the second target molecule; a second antibody or antigen-binding portion thereof against the second target molecule, wherein the second antibody or antigen-binding portion thereof is located on the surface of the LNP; or a second antibody or antigen-binding portion thereof against the second target molecule, wherein the second antibody or antigen-binding portion thereof is located on the surface of a second LNP. The present disclosure also provides a T cell targeted delivery system comprising (a) an LNP comprising a surface anchored T cell targeting molecule that specifically binds to a T cell specific surface protein, and (b) a costimulator of T cell activation. In some aspects, the T cell specific surface protein is CD3. In some aspects, the T cell targeting molecule comprises an anti CD3 antibody or antigen-binding portion thereof. In some aspects, the costimulator of T cell activation is a CD28 agonist. In some aspects, the CD28 agonist is an antibody that specifically binds CD28 or an antigen-binding portion thereof. In some aspects, the CD28 agonist is selected from the group consisting of an antibody that specifically binds CD28, a CD28 ligand, an aptamer, a peptide, a small molecule, or, a combination thereof. In some aspects, the CD28 ligand is B7-1 (CD80), B7-2 (CD86), or a combination thereof. In some aspects, the aptamer is CD28Apt7-dimer comprising a forward sequence of SEQ ID NO: 140, and a reverse sequence of SEQ ID NO: 141. In some aspects, the costimulator of T cell activation is an agonist of ICOS, B7, CD226, CRTAM, 41-BB, OX40, CD27, GITR, HVEM, CD40, BAFFR, BAFF, or a combination thereof. The present disclosure also provides a T cell targeted delivery system comprising (a) an LNP comprising a surface anchored T cell targeting molecule that specifically binds to one or two T cell specific surface proteins; or, (b) an LNP comprising one or two surface anchored T cell targeting molecules, wherein each T cell targeting molecule specifically binds to one or two T cell specific surface proteins; or, (c) a set of LNP comprising at least two LNPs, wherein the first LNP comprises a first T cell targeting molecule that specifically binds to a first T cell specific surface protein, and the second LNP comprises a second T cell targeting molecule that specifically binds to a second T cell specific surface protein; wherein the delivery system targets at least two T cell specific surface proteins, and wherein the LNP encapsulates a payload. In some aspects, the T cell specific surface proteins comprise CD3, and CD28. In some aspects, the T cell specific surface proteins comprise CD3 and a T cell specific surface protein selected from CD2, CD4, CD5, CD7, CD8, CD28, 4-1BB, NKG2D, or a combination thereof. In some aspects, the T cell specific surface proteins consist of CD3 and CD28, or CD3 and 4-1BB. In some aspects, the LNP comprises: (i) a cationic or ionizable cationic lipid or lipidoid; (ii) a structural lipid; (iii) a helper lipid; and, (iv) a stabilizing lipid. In some aspects, the ionizable cationic lipid or lipidoid is selected from the group consisting of cKK-E12, ALC-0315, SM-102, YK-009, DLin-MC3-DMA (MC3), DLin-KC2-DMA (KC2), A6, OF-02, A18-Iso5-2DC18, 98N₁₂−5, 9A1p9, C12-200, 7C1, G0-C14, L319, 304O₁₃, OF-Deg-Lin, 306-O12B, 306O110, FTT5, Lipid 8, Lipid 10, any one of MDX1-MDX13, and combinations thereof. In some aspects, the ionizable cationic lipid or lipidoid is cKK-E12 (3,6-bis(4-(bis(2-hydroxydodecyl)amino)butyl)piperazine-2,5-dione) or Lipid 10. In some aspects, the ionizable cationic lipid or lipidoid is DOTAP or DOTMA. In some aspects, the ionizable cationic lipid or lipidoid is Lipid 10. In some aspects, the cationic or ionizable cationic lipid or lipidoid is selected from the group consisting of MDX1-MDX13 (FIGS. 57-59), and combinations thereof.

The present disclosure also provides a T cell targeted delivery system comprising an LNP comprising a bispecific anti-CD3/anti-CD28 antibody anchored to the outer surface of the LNP and a payload encapsulated in the LNP, wherein the anti-CD3 portion of the bispecific antibody specifically binds to CD3 on the surface of a T cell and induces T cell uptake of the payload; and, the anti-CD28 portion of the bispecific antibody is a costimulator that specifically binds to CD28 on the surface of the T cell and increases expression of the payload; and, wherein the payload is a chimeric antigen receptor (CAR).

The present disclosure also provides a T cell targeted delivery system comprising an LNP comprising a monospecific anti-CD3 antibody and a monospecific anti-CD28 antibody anchored to the outer surface of the LNP and a payload encapsulated in the LNP, wherein the anti-CD3 antibody specifically binds to CD3 on the surface of a T cell and induces T cell uptake of the payload; and, the anti-CD28 antibody is a costimulator that specifically binds to CD28 on the surface of the T cell and increases expression of the payload; and, wherein the payload is a CAR.

In some aspects, the bispecific anti-CD3/anti-CD28 antibody comprises two polypeptides, each having a structure according to the formula VL-CL-Linker-VH-CH1-Fc, wherein VL is a light chain variable region, CL is a light chain constant region, VH is a heavy chain variable regions, CH1 is a heavy chain constant domain 1, and Fc is an Fc domain.

In some aspects, the anti-CD2 antibody and/or anti-CD8 antibody is covalently attached to the outer surface of the LNP. In some aspects, the antibodies anchored to the outer surface of the LNP are covalently attached to a lipid via maleimide chemistry. In some aspects, the bispecific anti-CD3/anti-CD28 antibody is MX1500 (SEQ ID NOS: 8 and 26). In some aspects, the bispecific anti-CD3/anti-CD28 antibody is MX1243 (SEQ ID NOS: 18 and 36). In some aspects, the monospecific anti-CD3 antibody is MX1507 (SEQ ID NOS: 5, 23, and 37). In some aspects, the monospecific anti-CD28 antibody is MX1506 (SEQ ID NOS: 7, 25, and 39). In some aspects, the anti-CD2 antibody is MX2864 (SEQ ID NOS: 567 and 568) and/or the anti-CD8 antibody is MX2862 (SEQ ID NOS: 559 and 560) or MX2863 (SEQ ID NOS: 563 and 564). In some aspects, the LNP comprises about 27.5 mol % of an ionizable cationic lipid (iLipid). In some aspects, the iLipid is selected from the group consisting of cKK-E12, MC3, SM-102, ACL-0315, KC2, Lipid A6, Lipid M, Lipid 10, C14-4, and any one of MDX1-MDX13. In some aspects, the T cell targeted delivery system comprises 27.5 mol % of iLipid; 16 mol % of DSPC; 2.45 mol % of DMG-PEG2000; 0.05 mol % of DSPE-PEG2000-maleimide; and 54 mol % of cholesterol. In some aspects, the CAR is a CD20-specific CAR. In some aspects, the CD20-specific CAR is RN105 (SEQ ID NO: 542). In some aspects, RN105 is encoded by the mRNA sequence set forth in SEQ ID NO: 543. In some aspects, the CAR is a CD79b-specific CAR. In some aspects, the CD79b-specific CAR is RN111 (SEQ ID NO: 545). In some aspects, RN111 is encoded by the mRNA sequence set forth in SEQ ID NO: 546. In some aspects, the CAR is a CD19-specific CAR. In some aspects, the CD19-specific CAR is RN068 (SEQ ID NO: 548). In some aspects, RN068 is encoded by the mRNA set forth in SEQ ID NO: 549. In some aspects, the CD19-specific CAR is RN082 (SEQ ID NO: 551). In some aspects, RN082 is encoded by the mRNA set forth in SEQ ID NO:552. In some aspects, the CD19-specific CAR is RN083 (SEQ ID NO:554). In some aspects, RN083 is encoded by the mRNA set forth in SEQ ID NO: 555. In some aspects, the CD19-specific CAR is RN084 (SEQ ID NO: 557). In some aspects, RN084 is encoded by the mRNA set forth in SEQ ID NO: 558.

The present disclosure also provides a T cell targeted delivery system comprising an LNP comprising a bispecific anti-CD3/anti-CD28 antibody anchored to the outer surface of the LNP and a payload encapsulated in the LNP, wherein the anti-CD3 portion of the bispecific antibody specifically binds to CD3 on the surface of a T cell and induces T cell uptake of the payload; and, the anti-CD28 portion of the bispecific antibody is a costimulator that specifically binds to CD28 on the surface of the T cell and increases expression of the payload; and, wherein the payload is a chimeric antigen receptor (CAR) selected from the group consisting of RN105 (SEQ ID NO: 542), RN111 (SEQ ID NO: 545), RN068 (SEQ ID NO: 548), RN082 (SEQ ID NO: 551), RN083 (SEQ ID NO: 554), and RN084 (SEQ ID NO: 557).

The present disclosure also provides a T cell targeted delivery system comprising an LNP comprising a bispecific anti-CD3/anti-CD28 antibody anchored to the outer surface of the LNP and a payload encapsulated in the LNP, wherein the anti-CD3 portion of the bispecific antibody specifically binds to CD3 on the surface of a T cell and induces T cell uptake of the payload; and, the anti-CD28 portion of the bispecific antibody is a costimulator that specifically binds to CD28 on the surface of the T cell and increases expression of the payload; wherein the payload is a chimeric antigen receptor (CAR) selected from the group consisting of a CD19-specific CAR, a CD20-specific CAR, and a CD79b-specific CAR; and, wherein the bispecific anti-CD3/anti-CD28 antibody comprises two polypeptides, each having a structure according to the formula VL-CL-Linker-VH-CH1-Fc, wherein VL is a light chain variable region, CL is a light chain constant region, VH is a heavy chain variable regions, CH1 is a heavy chain constant domain 1, and Fc is an Fc domain.

The present disclosure also provides a pharmaceutical composition comprising a T-cell target delivery system described herein and a pharmaceutically acceptable excipient.

The present disclosure also provides a method to treat a disease or condition comprising administering a T-cell targeted delivery system described herein or a pharmaceutical composition described herein to a subject in need thereof. In some aspects, the disease or condition is cancer.

The present disclosure also provides a lipid having the structure of any one of MDX1-MDX13. The present disclosure also provides a lipid nanoparticle (LNP) comprising the lipid of any one of MDX1-MDX13. The present disclosure also provides a T cell targeted delivery system comprising the lipid of any one of MDX1-MDX13, or a T cell targeted delivery system comprising an LNP comprising the lipid of any one of MDX1-MDX13. The present disclosure also provides a pharmaceutical composition comprising (i) the lipid of any one of MDX1-MDX13; (ii) an LNP comprising the lipid of any one of MDX1-MDX13; (iii) a T cell targeted delivery system comprising the lipid of any one of MDX1-MDX13; or (iv) a T cell targeted delivery system comprising an LNP comprising the lipid of any one of MDX1-MDX13; and a pharmaceutically acceptable excipient. The present disclosure also provides a method to treat a disease or condition comprising administering (i) a T cell targeted delivery system comprising the lipid of any one of MDX1-MDX13 or an LNP comprising the lipid of any one of MDX1-MDX13; or (ii) a pharmaceutical composition comprising the lipid of any one of MDX1-MDX13; an LNP comprising the lipid of any one of MDX1-MDX13; or a T cell targeted delivery system comprising the lipid of any one of MDX1-MDX13 or an LNP comprising the lipid of any one of MDX1-MDX13, to a subject in need thereof. In some aspects, the disease or condition is cancer.

### BRIEF DESCRIPTION OF THE DRAWINGS/FIGURES

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1A is a schematic of various anti-CD3 and anti-CD28 monovalent and bivalent antibody formats Format A, bivalent antibody; Format B, monovalent antibody; VH, heavy chain variable domain; VL, light chain variable domain; CL, light chain constant domain; CH1, heavy chain constant domain 1; CH2, heavy chain constant domain 2; CH3, heavy chain constant domain 3. FIG. 1B shows a schematic of various anti-CD3 and anti-CD28 bispecific antibody formats; Format C, bispecific aCD28/aCD3 antibody; Format D, single-arm aCD28 scFv/aCD3 scFv antibody; Format E, single-arm aCD28 scFv/aCD3 scFv-Fc molecule; Format F, aCD28 scFV/aCD3 scFv-Fc miniantibody; domain labels as in FIG. 1A.

FIG. 2 shows binding data to CD3 and CD28 for a control (IgG1LALAPA) and antibody constructs MX1243, MX1506, MX1507 and MX1500.

FIG. 3A shows a schematic of the composition of an LNP used for antibody conjugation for T cell targeting. FIG. 3B shows exemplary formats of combinations of antibody conjugated LNPs.

FIG. 4A shows a schematic for LNP production. FIG. 4B shows the structures of some of the lipid components of the LNPs.

FIG. 5A shows a schematic describing the maleimide group on the fatty acid chains of the outer surface of the LNP, and the thiol group on the C-terminus of an antibody. FIG. 5B shows the conjugation process between a LNP and an antibody by way of a maleimide-thiol reaction.

FIG. 6 shows a schematic for antibody-conjugated LNP production.

FIG. 7A shows a schematic for anti-CD3/CD28-conjugated LNPs (LNP/MX1243).

FIG. 7B shows the elution peak for the antibody-conjugated LNPs (LNP/MX1243) as compared to unconjugated antibodies (MX1243).

FIG. 8A shows the average hydrodynamic diameters (nm) for cKK-E12 LNPs (cKK-E12 LNP) and CD3/CD28 antibody-conjugated cKK-E12 LNPs (cKK-E12-LNP-CD3/CD28 antibody). FIG. 8B shows the difference in size and encapsulation efficiency of cKK-E12 LNP and cKK-E12 LNP-CD3/CD28 antibody.

FIG. 9 shows the consistency in size and encapsulation efficiency of various batches of antibody-conjugated LNPs. Different mRNA payloads (41.1 to 165.9 μg of mRNA per mL) and targeting antibody MX1243 (12.8 to 152.4 g per mL of anti-CD3/anti-CD28 targeting antibody) were used, corresponding to mRNA/MX1243 ratios between 1.1 and 3.2. Across these ranges of payloads and targeting molecules, encapsulation efficacy was close to 100%, and the size of the LNP was approximately 100 nm (99.55+/−4.82).

FIG. 10 shows the experimental design for determining T cell activation and GFP gene (SEQ ID NO: 534) delivery to PBMCs by anti-CD3/CD28-conjugated LNPs in vitro.

FIG. 11 shows the gating scheme used to evaluate mRNA delivery and T cell activation.

FIG. 12A shows the time course (1, 3, and 7 days post-treatment) of GFP mRNA (SEQ ID NO: 534) delivered to and expressed in viable total T cells using very low concentrations (0.0032 nM and 0.016 nM) of MX1243-conjugated LNPs relative to unconjugated control LNPs.

FIG. 12B shows the time course (1, 3, and 7 days post-treatment) of GFP mRNA (SEQ ID NO: 534) delivered to and expressed in viable total T cells using low concentrations (0.08 nM and 0.4 nM) of MX1243-conjugated LNPs relative to unconjugated control LNPs. FIG. 12C shows the time course (1, 3, and 7 days post-treatment) of GFP mRNA (SEQ ID NO: 534) delivered to and expressed in viable total T cells using high concentrations (2 nM and 10 nM) of MX1243-conjugated LNPs relative to unconjugated control LNPs.

FIG. 13A shows the time course (1, 3, and 7 days post-treatment) of GFP mRNA (SEQ ID NO: 534) delivered to and expressed in viable CD4+ T cells using very low concentrations (0.0032 nM and 0.016 nM) of MX1243-conjugated LNPs relative to unconjugated control LNPs. FIG. 13B shows the time course (1, 3, and 7 days post-treatment) of GFP mRNA (SEQ ID NO: 534) delivered to and expressed in viable CD4+ T cells using low concentrations (0.08 nM and 0.4 nM) of MX1243-conjugated LNPs relative to unconjugated control LNPs. FIG. 13C shows the time course (1, 3, and 7 days post-treatment) of GFP mRNA (SEQ ID NO: 534) delivered to and expressed in viable CD4+ T cells using high concentrations (2 nM and 10 nM) of MX1243-conjugated LNPs relative to unconjugated control LNPs.

FIG. 14A shows the time course (1, 3, and 7 days post-treatment) of GFP mRNA (SEQ ID NO: 534) delivered to and expressed in viable CD8+ T cells using very low concentrations (0.0032 nM and 0.016 nM) of MX1243-conjugated LNPs relative to unconjugated control LNPs. FIG. 14B shows the time course (1, 3, and 7 days post-treatment) of GFP mRNA (SEQ ID NO: 534) delivered to and expressed in viable CD8+ T cells using low concentrations (0.08 nM and 0.4 nM) of MX1243-conjugated LNPs relative to unconjugated control LNPs. FIG. 14C shows the time course (1, 3, and 7 days post-treatment) of GFP mRNA (SEQ ID NO: 534) delivered to and expressed in viable CD8+ T cells using high concentrations (2 nM and 10 nM) of MX1243-conjugated LNPs relative to unconjugated control LNPs.

FIG. 15A shows that MX1243-conjugated LNPs activate T cells at very low concentrations (0.0032 nM and 0.016 nM) at similar rates to a CD3-CD28 bispecific antibody control, as visualized by BcIXL. FIG. 15B shows that MX1243-conjugated LNPs activate T cells at very low concentrations (0.08 nM and 0.4 nM) at similar rates to a CD3-CD28 bispecific antibody control, as visualized by BcIXL. FIG. 15C shows that MX1243-conjugated LNPs activate T cells at high concentrations (2 nM and 10 nM) at similar rates to a CD3-CD28 bispecific antibody control, as visualized by BcIXL.

FIG. 16A shows that MX1243-conjugated LNPs activate T cells at very low concentrations (0.0032 nM and 0.016 nM) at similar rates to a CD3-CD28 bispecific antibody control, as visualized by CD25. FIG. 16B shows that MX1243-conjugated LNPs activate T cells at low concentrations (0.08 nM and 0.4 nM) at similar rates to a CD3-CD28 bispecific antibody control, as visualized by CD25. FIG. 16C shows that MX1243-conjugated LNPs activate T cells at high concentrations (2 nM and 10 nM) at similar rates to a CD3-CD28 bispecific antibody control, as visualized by CD25.

FIG. 17 shows an experimental design schematic for in vivo delivery of conjugated LNPs to circulating human primary T cells in NSG mice reconstituted with human PBMCs.

FIG. 18A shows a scatter plot indicating gating for the evaluation of GFP+ cells within viable T cell populations (Live, CD2⁺). FIG. 18B shows the frequency of GFP+ cells within viable T cell populations (Live, CD2⁺) in the blood at 16 hours post-treatment with LNP-MX1243 relative to unconjugated LNPs

FIG. 19A shows scatter plots of individual animals 16 hours after delivery of 1000 ng of unconjugated, GFP mRNA encapsulating LNPs into human CD2+ T cells in vivo. FIG. 19B shows scatter plots of individual animals 16 hours after delivery of 333 ng of MX1243-conjugated, GFP mRNA encapsulating LNPs into human CD2+ T cells in vivo. FIG. 19C shows scatter plots of individual animals 16 hours after delivery of 1000 ng of MX1243-conjugated, GFP mRNA encapsulating LNPs into human CD2+ T cells in vivo.

FIG. 20A shows scatter plots of splenocytes representing GFP+ cells within viable T cell populations (Live, CD2⁺). FIG. 20B shows the frequency of GFP+ cells within viable T cell populations (Live, CD2⁺) in the blood at 36 hours post-treatment with unconjugated, GFP mRNA encapsulating LNPs or MX1243 antibody-conjugated, GFP mRNA encapsulating LNPs (LNP-MX1243). FIG. 20C shows the frequency of GFP+ cells within viable T cell populations (Live, CD2⁺) in the spleen at 36 hours post-treatment with unconjugated, GFP mRNA encapsulating LNPs or MX1243 antibody-conjugated, GFP mRNA encapsulating LNPs (LNP-MX1243).

FIG. 21 shows schematics of two bivalent monospecific antibodies (MX1506 and MX1507) and two bispecific antibodies (MX1243 and MX1500).

FIG. 22 is a schematic representation of the mixture of monospecific antibodies MX1506 and MX1507 prior to LNP preparation to generate bispecific LNPs.

FIG. 23 shows a schematic of single antibodies conjugated separately to LNPs (monovalent LNPs) and then mixed to generate a bispecific LNP mixture.

FIG. 24 present quality control data corresponding to specific combinations of antibodies and different LNPs. All the LNPs contained the same payload (mRNA encoding EGFP).

FIG. 25 shows the efficacy of mRNA delivery corresponding to different LNP compositions (LNP1 to LNP-11) administered at 4 different concentrations to T cells from two human donors.

FIG. 26 shows the efficacy of T cell activation, measured by quantitating CD69 expression, corresponding to different LNP compositions (LNP1 to LNP-11) administered at 4 different concentrations to T cells from two human donors.

FIG. 27 shows the upregulation of the anti-apoptotic protein Bcl-xL on viable T cell populations (Live, CD2⁺) of Donor 1 (top) and Donor 2 (bottom) after treatment with LNP-1 to LNP-11 compared to unconjugated LNPs.

FIG. 28 is a schematic representation of a CAR showing its domain organization. The binding domain can be, e.g., a single chain variable fragment (scFv) in the V_{H}-V_{L} or V_{L}-V_{H} orientation, or any antigen-binding portion of an antibody or combination thereof. The ectodomain comprises an antigen-binding domain and a CAR spacer. The endodomain can also be referred to as an intracellular or intracytoplasmic domain and can comprise an immunoreceptor tyrosine-based activation motif (ITAM) and optional costimulatory modules.

FIG. 29 is a schematic representation showing the differences in structure between different generations of CARs. First generation CARs comprise an antigen-binding domain (e.g., a scFv) connected to ITAM directly via a transmembrane region. Second generation CARs comprise a costimulatory molecule (CM1) interposed between ITAM and the transmembrane domain. Third generation CARs include an additional costimulatory molecule (CM2). In fourth generation CARs a costimulatory molecule has been replaced with an IL-12 inducer. Fifth generation CARs are based on second generation CARs, but they contain a truncated cytoplasmic IL-2 receptor 3-chain domain.

FIG. 30 shows a schematic representation of a third generation CAR having two antigen targeting specificities, TAA1 and TAA2, e.g., anti-CD19 and anti-CD20, and the sequences of second generation and third generation bispecific CARs targeting CD19 and CD20, including their domain organization.

FIG. 31 shows exemplary ionizable cationic lipids that can be used in the LNP of the present disclosure. See, e.g., Naidu et al. Adv. Sci. 10.19 (2023): 2301929; Ramishetti et al. Adv. Mater. 32.12 (2020): 1906128; and U.S. Pat. No. 11,851,389B2, which are herein incorporated by reference in their entireties.

FIG. 32 shows cargo delivery efficacy to human T cells by an anti-CD3/CD28 bispecific antibody-conjugated LNP formulated with different ionizable cationic lipids (ilipids). To assess the suitability of different ionizable cationic lipids for antibody conjugation, various ilipids were tested in ilipid/DSPC/Chol/PEG2000-DMG/DSPE-PEG2000-maleimide LNP formulations with 35/16/46.5/2.3/0.2 molar % using EGFP mRNA as cargo. The conjugated LNP were evaluated by transfecting unstimulated human PBMCs at indicated EGFP mRNA payload concentrations. At 20 hours post transfection, PBMC's were subjected to flow cytometry analysis to assess mRNA delivery to T-cell populations (Live, CD2⁺) by percentage of GFP expressing cells. The result demonstrated that Lipid 10 and SM102 based LNP showed the highest delivery efficacy.

FIG. 33 is a graphic representation of an exemplary antibody-conjugated LNP production pipeline.

FIG. 34 is a graphic representation of cysteine capping and de-capping using reduction agents such as DTT or TCEP.

FIG. 35A shows mRNA delivery in vitro to unstimulated human PBMCs by LNP-antibody conjugates with different molar % of Lipid 10 (donor 1). At 20 hours post dosing, PBMC's were subjected to flow cytometry analysis to assess mRNA delivery (GFP expression) to T cell populations (Live, CD2⁺). FIG. 35B shows mRNA delivery in vitro to unstimulated human PBMCs by LNP-antibody conjugates with different molar % of Lipid 10 (donor 2). At 20 hours post dosing, PBMC's were subjected to flow cytometry analysis to assess mRNA delivery (GFP expression) to T cell populations (Live, CD2⁺). FIG. 35C shows mRNA delivery in vitro to unstimulated human PBMCs by LNP-antibody conjugates with different molar % of Lipid 10 (MFI assessment) (donor 1). At 20 hours post dosing, PBMC's were subjected to flow cytometry analysis to assess mRNA delivery (GFP expression) to T cell populations (Live, CD2⁺). FIG. 35D shows mRNA delivery in vitro to unstimulated human PBMCs by LNP-antibody conjugates with different molar % of Lipid 10 (MFI assessment) (donor 2). At 20 hours post dosing, PBMC's were subjected to flow cytometry analysis to assess mRNA delivery (GFP expression) to T cell populations (Live, CD2⁺). FIG. 35E shows mRNA delivery in vitro to unstimulated human PBMCs by LNP-antibody conjugates with different molar % of Lipid 10 (CD4 T cells) (donor 1). At 20 hours post dosing, PBMC's were subjected to flow cytometry analysis to assess mRNA delivery (GFP expression) to T cell populations (Live, CD4⁺). FIG. 35F shows mRNA delivery in vitro to unstimulated human PBMCs by LNP-antibody conjugates with different molar % of Lipid 10 (CD4 T cells) (donor 2). At 20 hours post dosing, PBMC's were subjected to flow cytometry analysis to assess mRNA delivery (GFP expression) to T cell populations (Live, CD4⁺). FIG. 35G shows mRNA delivery in vitro to unstimulated human PBMCs by LNP-Antibody Conjugates with different molar % of Lipid 10 (CD8 T cells)(donor 1). At 20 hours post dosing, PBMC's were subjected to flow cytometry analysis to assess mRNA delivery (GFP expression) to T cell populations (Live, CD8⁺). FIG. 35H shows mRNA delivery in vitro to unstimulated human PBMCs by LNP-Antibody Conjugates with different molar % of Lipid 10 (CD8 T cells)(donor 2). At 20 hours post dosing, PBMC's were subjected to flow cytometry analysis to assess mRNA delivery (GFP expression) to T cell populations (Live, CD8⁺).

FIG. 36 shows mRNA delivery to peripheral human T cell populations in a human PBMC reconstituted NSG-MHC I/II DKO mutant mice in vivo by antibody conjugated Lipid 10/EGFP LNPs. 0.2 g mRNA payload/mouse of indicated Lipid 10 formulated LNP conjugated with MX1500 was administered intravenously and 50 μl of blood was collected from each mouse 16 hours post LNP delivery. Whole blood was subjected to flow cytometry analysis to assess mRNA delivery (EGFP expression) to peripheral human T cells populations (Live, hCD2⁺) and live murine leukocytes populations (Live, mCD45⁺).

FIG. 37A shows mRNA delivery and activation of human T cells in vitro by LNP-antibody conjugates (Donor 1) with different molar % of the DSPE-PEG2000-Maleimide. At 20 hours post dosing, PBMC's were subjected to flow cytometry analysis to assess mRNA delivery (GFP expression) and T cell activation (CD69 expression) to T cell populations (Live, CD2⁺). FIG. 37B shows mRNA delivery and activation of human T cells in vitro by LNP-antibody conjugates (Donor 2) with different molar % of the DSPE-PEG2000-Maleimide. At 20 hours post dosing, PBMC's were subjected to flow cytometry analysis to assess mRNA delivery (GFP expression) and T cell activation (CD69 expression) to T cell populations (Live, CD2⁺).

FIG. 38A shows mRNA delivery and activation of human CD4 T cells in vitro by LNP-antibody conjugates (Donor 1) with different molar % of the DSPE-PEG2000-Maleimide. At 20 hours post dosing, PBMC's were subjected to flow cytometry analysis to assess mRNA delivery (GFP expression) and T cell activation (CD69 expression) to CD4 T cell population (Live, CD2⁺CD4⁺). FIG. 38B shows mRNA delivery and activation of human CD4 T cells in vitro by LNP-antibody conjugates (Donor 2) with different molar % of the DSPE-PEG2000-Maleimide. At 20 hours post dosing, PBMC's were subjected to flow cytometry analysis to assess mRNA delivery (GFP expression) and T cell activation (CD69 expression) to CD4 T cell population (Live, CD2⁺CD4⁺).

FIG. 39A shows mRNA delivery and activation of human CD8 T cells in vitro by LNP-antibody conjugates (Donor 1) with different molar % of the DSPE-PEG2000-Maleimide. At 20 hours post dosing, PBMC's were subjected to flow cytometry analysis to assess mRNA delivery (GFP expression) and T cell activation (CD69 expression) to CD4 T cell population (Live, CD2⁺CD8⁺). FIG. 39B shows mRNA delivery and activation of human CD8 T cells in vitro by LNP-antibody conjugates (Donor 2) with different molar % of the DSPE-PEG2000-Maleimide. At 20 hours post dosing, PBMC's were subjected to flow cytometry analysis to assess mRNA delivery (GFP expression) and T cell activation (CD69 expression) to CD4 T cell population (Live, CD2⁺CD8⁺).

FIG. 40 shows EGFP total expression level (MFI) to human T cells in vitro by LNP-antibody conjugates (Donors 1 & 2) with different molar % of the DSPE-PEG2000-Maleimide. At 20 hours post dosing, PBMC's were subjected to flow cytometry analysis to assess mRNA expression level (MFI) to CD2 T cell population (Live, CD2+.

FIG. 41A shows mRNA delivery to peripheral human T cell populations in human PBMC reconstituted NSG-MHC I/I DKO mice in vivo by antibody conjugated Lipid 10/EGFP LNPs using various molar % of DSPE-PEG2000-Maleimide. 0.2 g mRNA payload/mouse of indicated Lipid 10 formulated LNP conjugated with MX1500 was administered intravenously and 50 μL of blood was collected from each mouse 16 hours post LNP delivery. Whole blood was subjected to flow cytometry analysis to assess mRNA delivery (EGFP expression) to peripheral human T cells populations (Live, hCD2⁺) and live murine leukocytes populations (Live, mCD45⁺). Data presented either as percentage of CD2⁺EGFP⁺ among total CD2+ cells (left panel) or as mean fluorescence intensity (MFI) of total CD2⁺EGFP⁺ cells (right panel). FIG. 41B shows mRNA delivery to peripheral human T cell populations in human PBMC reconstituted NSG-MHC I/II DKO mice in vivo by antibody conjugated Lipid 10/EGFP LNPs using various molar % of DSPE-PEG2000-Maleimide. 0.2 μg mRNA payload/mouse of indicated Lipid 10 formulated LNP conjugated with MX1500 was administered intravenously and 50 μL of blood was collected from each mouse 40 hours post LNP delivery. Whole blood was subjected to flow cytometry analysis to assess mRNA delivery (EGFP expression) to peripheral human T cells populations (Live, hCD2⁺) and live murine leukocytes populations (Live, mCD45⁺). Data presented either as percentage of CD2⁺EGFP⁺ among total CD2+ cells (left panel) or as mean fluorescence intensity (MFI) of total CD2⁺EGFP⁺ cells (right panel).

FIG. 42 shows mRNA delivery and activation of human T cells in vitro by LNP-antibody conjugates (Donor 1) with different molar % of the DSPC. At 20 hours post dosing, PBMC's were subjected to flow cytometry analysis to assess mRNA delivery (GFP expression) and T cell activation (CD69 expression) to T cell populations (Live, CD2⁺).

FIG. 43A shows mRNA delivery to human T cells in vitro by LNP-antibody conjugates (Donor 1) with N/P ratio of 4.5, 7, 9. At 20 hours post dosing, PBMCs were subjected to flow cytometry analysis to assess mRNA delivery (EGFP expression) to peripheral human T cells populations (Live, hCD2⁺) Data presented either as percentage of CD2+EGFP+ among total CD2+ cells (left panel) or as mean fluorescence intensity (MFI) of total CD2+EGFP+ cells (right panel).

FIG. 43B shows activation of human T cells in vitro by LNP-antibody conjugates (Donor 1) with N/P ratio of 4.5, 7, 9. At 20 hours post dosing, PBMC's were subjected to flow cytometry analysis to assess T cell activation (CD69 expression) to T cell populations (Live, CD2⁺).

FIG. 44 shows the MSTAR and MSTAR v1 architectures. Preferred linker lengths between the components of the MSTAR constructs are indicated in red.

FIG. 45A shows the domain organization of the sequence of the MX1500 bispecific antibody knob chain. FIG. 45B shows the domain organization of the sequence of the MX1500 bispecific antibody hole chain.

FIG. 46 shows an exemplary workflow to produce a Lipid 10-optimized LNPs comprising antiCD3/antiCD28 bispecific antibody targeting moieties attached to their surface, and carrying as payload an mRNA encoding a CAR.

FIG. 47 shows the chemical structures of ionizable cationic lipids.

FIG. 48 shows the chemical structures of commercially available LNP formulations used in Example 11.

FIG. 49A is a schematic representation of the CAR constructs used for UTR optimization for CAR expression in human T cells in vitro. FIG. 49B shows the domain architecture of the four constructs used in the UTR optimization process, named MD3385, MD3495, MD3496 and MD3497. FIG. 49C shows the biophysical properties of unconjugated and conjugated LNP used in the UTR optimization process. FIG. 49D shows the expression levels of CD19 CARs with different UTRs. The combination of human β-globin (hBB) 5′ UTR and human β-globin (hBB) 3′ UTR showed the highest level of CD19 CAR surface expression.

FIG. 50A shows the structure of a CD20 CAR and CD 79b CAR used to optimize expression in human PBMCs following encapsulation in conjugated SM102-based LNPs (LNPs with a MX1500 targeting antibody covalently attached to their surfaces via maleimide chemistry). FIG. 50B shows cell surface expression of human CD20 CARs from hPBMCs transfected by MX1500 conjugated and non-conjugated SM102-based LNPs. FIG. 50C shows cell surface expression of human CD79b CAR from hPBMCs transfected by MX1500 conjugated and non-conjugated SM102-based LNPs.

FIG. 51 shows the architecture of the anti-CD2 and CD8 monoclonal antibodies used in anti-CD3/anti-CD28 targeting studies. The anti-CD2 or anti-CD8 were conjugated to SM102-based LNPs with RN068 mRNA cargo (mRNA of SEQ ID NO: 549 encoding the anti-CD19 CAR of SEQ ID NO: 548).

FIG. 52 shows CD19CAR expression in CD4 T cells delivered by anti-CD2 conjugated LNPs, anti-CD8 conjugated LNPs, and anti-CD3/CD28 conjugated LNPs.

FIG. 53 shows CD4 T-cell activation using anti-CD2 conjugated LNPs, anti-CD8 conjugated LNPs, and anti-CD3/CD28 conjugated LNPs with RN068 mRNA cargo (mRNA of SEQ ID NO: 549 encoding the anti-CD19 CAR of SEQ ID NO: 548).

FIG. 54 shows the delivery efficacy to CD8 T cells using anti-CD2 conjugated LNPs, anti-CD8 conjugated LNPs, and anti-CD3/CD28 conjugated LNPs with RN068 mRNA cargo (mRNA of SEQ ID NO: 549 encoding the anti-CD19 CAR of SEQ ID NO: 548).

FIG. 55 shows the degree of CD8 T-cell activation using anti-CD2 conjugated LNPs, anti-CD8 conjugated LNPs, and anti-CD3/CD28 conjugated LNPs with RN068 mRNA cargo (mRNA of SEQ ID NO: 549 encoding the anti-CD19 CAR of SEQ ID NO: 548). The delivery efficiency correlated with degree of CD8 T cell activation.

FIG. 56 shows the overall cargo delivery capacity to human T cells of anti-CD2 conjugated LNPs, anti-CD8 conjugated LNPs, and anti-CD3/CD28 conjugated LNPs with RN068 mRNA cargo (mRNA of SEQ ID NO: 549 encoding the anti-CD19 CAR of SEQ ID NO: 548). Anti-CD3/CD28 demonstrated significantly higher cargo delivery capacity to human primary T cells in vitro, compared to delivery mediated by anti-CD2 or anti-CD8.

FIG. 57 shows the chemical structures of ionizable lipids MDX1-MDX4.

FIG. 58 shows the chemical structures of carbohydrate-based ionizable lipids MDX5-MDX7.

FIG. 59 shows the chemical structures of ionizable lipid 14 serials MDX8-MDX13.

### DETAILED DESCRIPTION

The present disclosure relates to delivering target therapeutic or diagnostic payloads (e.g., mRNAs, antibodies, therapeutic oligonucleotides, etc.) to specific cells or tissues, e.g., human T cells, by conjugating monospecific, bispecific or multispecific binding molecules (e.g., antibodies) recognizing at least one cell or tissue specific (e.g., T cell specific) surface protein to LNPs.

In the case of T cells, such surface proteins can be, for example, CD2, CD3, CD4, CD5, CD8, 4-1BBL, OX40L, or CD28. Proteins that are predominantly expressed on the surface of specific cell types (e.g., B cells, muscle cells, neurons, cancer cells, etc.) can be targeted by a ligand or set of ligands (e.g., antibodies) attached to the surface of LNPs disclosed in the present specification.

The conjugation of binding molecules can be performed, for example, by click chemistry, attaching the antibody or antibodies to a lipid component of a payload carrier such as an LNP having a PEG moiety (for example, PEG2000-maleimide) of a PEGylated lipid or other functional group that can bind to a functional group on a T cell targeting antibody.

The conjugated LNPs should preferentially bind to the targeted cell or tissue, e.g., T cells. Effective transfer of primary human T cells requires activation and proliferation signals. It is well established that anti-CD3 provides the primary T cell activation signal, and anti-CD28 offers a second signal for better T cell survival and proliferation. T cell activation can also facilitate the uptake of LNPs. Thus, in some aspects, the LNPs of the present disclosure comprise anti-CD3 and anti-CD28 mono or bispecific antibodies. In some particular aspects, the present disclosure provided unconjugated and conjugated LNP comprising the Lipid 10 ionizable cationic lipid.

Various terms relating to aspects of disclosure are used throughout the specification and claims. Such terms are to be given their ordinary meaning in the art, unless otherwise indicated. Other specifically defined terms are to be construed in a manner consistent with the definitions provided herein.

### Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 15 percent, up or down (higher or lower). Thus, in some aspects, about is interchangeable with ±15%. When particular values or compositions are provided in the application and claims, unless otherwise stated, the meaning of “about” should be assumed to be within an acceptable error range for that particular value or composition.

As described herein, any numerical range, concentration range, percentage range, ratio range or integer range is to be understood to include the value of any integer within the recited range and, when appropriate, fractions thereof (such as one-tenth and one-hundredth of an integer), unless otherwise indicated.

As used herein, the term “antigen binding polypeptide” refers to a polypeptide having the ability to specifically bind to one or more substances that induce an immune response (i.e., one or more antigens or epitopes).

As used herein, the term “antigen binding polypeptide complex” refers to a group of two, three, four, or more associated polypeptides, wherein at least one polypeptide has the ability to specifically bind to one or more antigens. An antigen binding polypeptide complex, includes, but is not limited to, an antibody or antigen binding fragment thereof.

The term “antibody” includes, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

The term “monoclonal antibody,” as used herein, refers to an antibody that is produced by a single clone of B-cells and binds to the same epitope. In contrast, the term “polyclonal antibody” refers to a population of antibodies that are produced by different B-cells and bind to different epitopes of the same antigen. The term “antibody” includes, by way of example, monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man.

The antibody can be an antibody that has been altered (e.g., by mutation, deletion, substitution, conjugation to a non-antibody moiety). For example, an antibody can include one or more variant amino acids (compared to a naturally occurring antibody) which change a property (e.g., a functional property) of the antibody. For example, several such alterations are known in the art, which affect, e.g., half-life, effector function, and/or immune responses to the antibody in a patient. The term antibody also includes artificial polypeptide constructs, which comprise at least one antibody-derived antigen-binding site.

An “antigen binding fragment” of an antibody refers to one or more fragments or portions of an antibody that retain the ability to bind specifically to the antigen bound by the whole antibody. It has been shown that the antigen binding function of an antibody can be performed by fragments or portions of a full-length antibody. An antigen-binding fragment can contain the antigenic determining regions of an intact antibody (e.g., the complementarity determining regions (CDRs)). Examples of antigen binding fragments of antibodies that can be used in the targeted delivery systems of the present disclosure include, but are not limited to, Fab, Fab′, F(ab′)₂, scFv, and Fv fragments, linear antibodies, and single chain antibodies. An antigen-binding fragment of an antibody can be derived from any animal species, such as rodents (e.g., mouse, rat, or hamster) and humans or can be artificially produced.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody.

Antigen binding fragments are obtained using conventional techniques known to those with skill in the art, and the fragments screened for utility in the same manner as are intact antibodies. Antigen binding fragments can be produced by recombinant DNA techniques, or by enzymatic or chemical cleavage of intact immunoglobulins.

As used herein, the term “variable region” typically refers to a portion of an antibody, generally, a portion of a light or heavy chain, typically about the amino-terminal 110 to 120 amino acids, or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called Complementarity Determining Regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of an antibody with antigen. In some aspects, the variable region is a mammalian variable region, e.g., a human, mouse or rabbit variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “complementarity determining region” or “CDR”, as used herein, refer to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL”, “VL region,” and “VL domain” are used herein interchangeably to refer to the light chain variable region of an antigen binding polypeptide, antigen binding polypeptide complex, antibody or antigen binding fragment thereof. In some aspects, a VL region is referred to herein as VL1 to denote a first light chain variable region, VL2 to denote a second light chain variable region, VL3 to denote a third light chain variable region, and VL4 to denote a fourth light chain variable region. An enumerated VL region (e.g., VL1) can have the same or different antigen binding properties and/or the same or different sequence as another enumerated VL region (e.g., VL2).

The terms “VH”, “VH region,” and “VH domain” are used herein interchangeably to refer to the heavy chain variable region of an antigen binding polypeptide, antigen binding polypeptide complex, antibody or antigen binding fragment thereof. In some aspects, a VH region is referred to herein as VH1 to denote a first heavy chain variable region, VH2 to denote a second heavy chain variable region, VH3 to denote a third heavy chain variable region, and VH4 to denote a fourth heavy chain variable region. An enumerated VH region (e.g., VH1) can have the same or different antigen binding properties and/or the same or different sequence as another enumerated VH region (e.g., V12).

As used herein, “Kabat numbering” and like terms are recognized in the art and refer to a system of numbering amino acid residues in the heavy and light chain variable regions of an antibody or antigen binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the terms “constant region” or “constant domain” are used interchangeably to refer to a portion of an antigen binding polypeptide, antigen binding polypeptide complex, antibody or antigen binding fragment thereof, e.g., a carboxyl terminal portion of a light and/or heavy chain which is not directly involved in binding of an antibody to antigen but which can exhibit various effector functions, such as interaction with the Fc region. The constant region generally has a more conserved amino acid sequence relative to a variable region. In some aspects, an antigen binding polypeptide, antigen binding polypeptide complex, antibody or antigen binding fragment thereof comprises a constant region or portion thereof that is sufficient for antibody-dependent cell-mediated cytotoxicity (ADCC), antibody-dependent cellular phagocytosis (ADCP), and complement-dependent cytotoxicity (CDC). A constant region includes, but is not limited to, a light chain constant region (CL) or heavy chain constant region (CH1, CH2, CH3, or a combination thereof, e.g., CH1-CH2 or CH2-CH3).

As used herein, the terms “fragment crystallizable region,” “Fc region,” or “Fc domain” are used interchangeably herein to refer to the tail region of an antibody that interacts with cell surface receptors called Fc receptors and some proteins of the complement system. Fc regions typically comprise CH2 and CH3 regions, and, optionally, an immunoglobulin hinge.

As used herein, the terms “immunoglobulin hinge,” “hinge,” “hinge domain” or “hinge region” are used interchangeably to refer to a stretch of heavy chains between the Fab and Fc portions of an antigen binding polypeptide, antigen binding polypeptide complex, antibody or antigen binding fragment thereof. A hinge provides structure, position and flexibility, which assist with normal functioning of antibodies (e.g., for crosslinking two antigens or binding two antigenic determinants on the same antigen molecule). An immunoglobulin hinge is divided into upper, middle and lower hinge regions that can be separated based on structural and/or genetic components. An immunoglobulin hinge of the invention can contain one, two or all three of these regions. Structurally, the upper hinge region stretches from the C terminal end of CH1 to the first hinge disulfide bond. The middle hinge region stretches from the first cysteine to the last cysteine in the hinge. The lower hinge region extends from the last cysteine to the glycine of CH2. The cysteines present in the hinge form interchain disulfide bonds that link the immunoglobulin monomers.

As used herein, the term “Fab” refers to a region of an antibody that binds to an antigen. It is typically composed of one constant and one variable domain of each of the heavy and the light chain.

As used herein, the term “heavy chain” refers to a portion of an antigen binding polypeptide, antigen binding polypeptide complex, antibody or antigen binding fragment thereof typically composed of a heavy chain variable region (VH), a heavy chain constant region 1 (CH1), a heavy chain constant region 2 (CH2), and a heavy chain constant region 3 (CH3). A typical antibody is composed of two heavy chains and two light chains. When used in reference to an antibody, a heavy chain can refer to any distinct type, e.g., alpha (α), delta (δ), epsilon (ε), gamma (γ), and mu (μ), based on the amino acid sequence of the constant region, which gives rise to IgA, IgD, IgE, IgG, and IgM classes of antibodies, respectively, including subclasses of IgG, e.g., IgG1, IgG2, IgG3, and IgG4. Heavy chain amino acid sequences are known in the art. In some aspects, the heavy chain is a human heavy chain.

As used herein, the term “light chain” refers to a portion of an antigen binding polypeptide, antigen binding polypeptide complex, antibody or antigen binding fragment thereof typically composed of a light chain variable region (VL) and a light chain constant region (CL). A typical antibody is composed of two light chains and two heavy chains. When used in reference to an antibody, a light chain can refer to any distinct type, e.g., kappa (κ) or lambda (λ), based on the amino acid sequence of the constant region. Light chain amino acid sequences are known in the art. In some aspects, the light chain is a human light chain.

The term “chimeric” antibody or antigen-binding fragment thereof refers to an antibody or antigen binding fragments thereof wherein the amino acid sequence is derived from two or more species. Typically, the variable region of both light and heavy chains corresponds to the variable region of antibodies or antigen binding fragments thereof derived from one species of mammals (e.g., mouse, rat, rabbit, etc.) with the desired specificity, affinity and capability, while the constant regions are homologous to the sequences in antibodies or antigen binding fragments thereof derived from another (usually human) to avoid eliciting an immune response in that species.

The term “humanized” antibody or antigen binding fragment thereof refers to forms of non-human (e.g., murine) antibodies or antigen binding fragments that are specific immunoglobulin chains, chimeric immunoglobulins, or fragments thereof that contain minimal non-human (e.g., murine) sequences. Typically, humanized antibodies or antigen binding fragments thereof are human immunoglobulins in which residues from a complementary determining region (CDR) are replaced by residues from a CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen binding fragment thereof can be further modified by the substitution of additional residues either in the Fv framework region and/or within the replaced non-human residues to refine and optimize antibody or antigen-binding fragment thereof specificity, affinity, and/or capability. In general, a humanized antibody or antigen binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. A humanized antibody or antigen binding fragment thereof can also comprise at least a portion of a constant region, typically that of a human immunoglobulin. Examples of methods used to generate humanized antibodies are known and described, for example, in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody or antigen-binding fragment thereof, as used herein, means an antibody or antigen-binding fragment thereof having an amino acid sequence derived from a human immunoglobulin gene locus, where such antibody or antigen-binding fragment is made using recombinant techniques known in the art. This definition of a human antibody or antigen-binding fragment thereof includes intact or full-length antibodies and fragments thereof.

A polypeptide, polypeptide complex, antibody, antigen binding fragment thereof, polynucleotide, vector or host cell which is “isolated” is a polypeptide, polypeptide complex, antibody, antigen binding fragment thereof, polynucleotide, vector or host cell which is in a form not found in nature. Isolated polypeptides, polypeptide complexes, antibodies, antigen binding fragments thereof, polynucleotides, vectors or host cells include those which have been purified to a degree that they are no longer in a form in which they are found in nature. In some aspects, a polypeptide, polypeptide complex, antibody, antigen-binding fragment thereof, polynucleotide, vector or host cell, which is isolated, is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

As used herein, the terms “identity” and “sequence identity” are used interchangeably and refer to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent of sequence identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In some aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions are then compared.

When a position in the first sequence is occupied by the same amino acid as the corresponding position in the second sequence, then the molecules are identical at that position. The percentage of sequence identity between the two sequences is a function of the number of identical positions shared by the sequences, taking into account the number of gaps, and the length of each gap, which needs to be introduced for optimal alignment of the two sequences. The comparison of sequences and determination of percentage of sequence identity between two sequences can be accomplished using a mathematical algorithm.

Suitable software programs are available from various sources, and for alignment of both protein and nucleotide sequences. One suitable program to determine percentage of sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percentage of sequence identity. It is noted that the percentage of sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In some aspects, the percentage of sequence identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percentage of sequence identity of the first sequence to the second sequence will be higher than the percentage of sequence identity of the second sequence to the first sequence.

One skilled in the art will appreciate that the generation of a sequence alignment for the calculation of a percentage of sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percentage of sequence identity can be curated either automatically or manually.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In some aspects, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects of the present disclosure, the biologically active molecule is a polynucleotide.

In some aspects, a polynucleotide disclosed herein can be modified to introduce a thiol group that could be used to react with a maleimide moiety. In some aspects, a polynucleotide disclosed herein can be modified to introduce a maleimide moiety group that could be used to react with a thiol group.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, about 10, about 15, about 20, about 25, about 30, about 35, about 40, about 45, or about 50 amino acids long.

In some aspects, a polypeptide disclosed herein can be modified to introduce a thiol group that could be used to react with a maleimide moiety. In some aspects, a polypeptide disclosed herein can be modified to introduce a maleimide moiety that could be used to react with a thiol group.

As used herein, the term “nanoparticle” refers to particles having a particle size on the nanometer scale, less than 1 micrometer. For example, the nanoparticle may have a particle size up to about 50 nm. In another example, the nanoparticle may have a particle size up to about 10 nm. In another example, the nanoparticle may have a particle size up to about 6 nm. As used herein, “nanoparticle” refers to a number of nanoparticles, including, but not limited to, nanoclusters, nanovesicles, micelles, lamellae shaped particles, polymersomes, dendrimers, and other nano-size particles of various other small fabrications that are known to those in the art. The shapes and compositions of nanoparticles may be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application.

As used herein, the term “Chimeric Antigen Receptor” or alternatively a “CAR” refers to a recombinant polypeptide construct comprising at least an extracellular antigen binding domain, a transmembrane domain, and a cytoplasmic signaling domain comprising a functional signaling domain derived from a stimulatory molecule as defined below. In some aspects, the stimulatory molecule is the zeta chain associated with the T cell receptor complex. In some aspects, the cytoplasmic signaling domain further comprises one or more functional signaling domains derived from at least one costimulatory molecule as defined below. In some aspects, the costimulatory molecule is chosen from 4-1BB (i.e., CD137), CD3, and/or CD28. In some aspects, the CAR comprises a chimeric fusion protein comprising an extracellular antigen recognition domain, a transmembrane domain, and an intracellular signaling domain comprising a functional signaling domain derived from a stimulatory molecule. In some aspects, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In some aspects, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In some aspects, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In some aspects, the CAR comprises an optional leader sequence at the amino-terminus (N-ter) of the CAR fusion protein. In some aspects, the CAR further comprises a leader sequence at the N-terminus of the extracellular antigen recognition domain, wherein the leader sequence is optionally cleaved from the scFv domain during cellular processing and localization of the CAR to the cellular membrane.

The portion of the CAR composition comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the antigen-binding domain of a CAR composition of the invention comprises an antibody fragment. In some aspects, the CAR comprises an antibody fragment that comprises a scFv. In some aspects, a CAR is a payload within an LNP having a targeting antibody or combination thereof bound thereto.

In some aspects, the targeting portion of the CAR is in “MSTAR” format, e.g., an antibody format disclosed in U.S. Appl. Publ. Nos. US20230227553A1, US20230235092A1, US20230203199A1, and PCT Publ. Nos. WO2023056312, WO2023056313, WO2023056314, which are herein incorporated by reference in their entireties. Exemplary antibodies in MSTAR format are presented in Section I.C.i “Bispecific Antibody Architectures” of this disclosure. In some aspects, the targeting portion of the antibody is a bispecific MSTAR antibody. In some aspects, the bispecific MSTAR antibody targets CD19 and CD20. In some aspects, the bispecific MSTAR antibody targets CD3 and CD28.

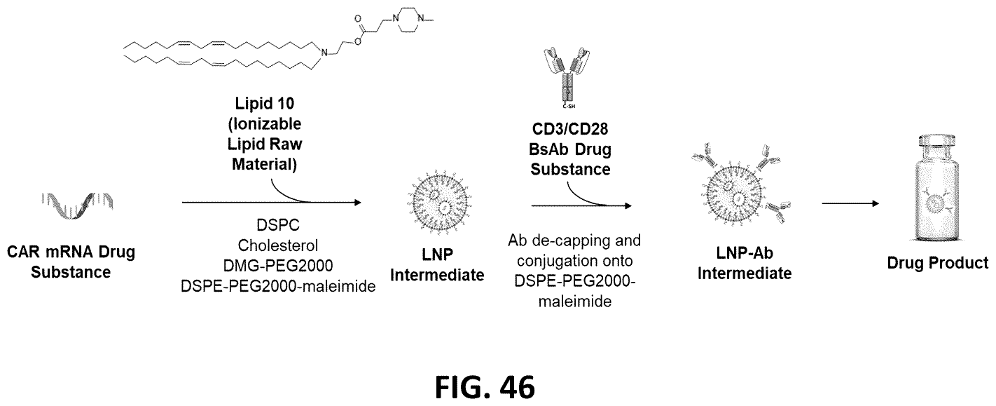

As used herein, the term “MSTAR” refers to an antibody format presented in Section I.C.i “Bispecific Antibody Architectures” of the present disclosure. In some aspects, the term MSTAR refers to the architectures presented in FIG. 46. The MSTAR design combines heavy and light regions into a single chain and stitches together two (or more) different antibody specificities onto one antibody “arm”.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “lipid” refers to a group of organic compounds that include, but are not limited to, esters of fatty acids and are characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids. The selection of the individual lipid components of the lipid formulation is made to optimize delivery of a payload (e.g., a nucleic acid) to a target cell. As used herein, the phrase “lipid formulation” refers to a formulation comprising one or more lipids (e.g., cationic lipids, non-cationic lipids, lipid conjugates, and the like).

As used herein, the term “lipid nanoparticle” is used interchangeably with the abbreviation “LNP” and refers to a microscopic lipid formulation that can be used to deliver an active agent or therapeutic agent, such as a nucleic acid (e.g., an mRNA, dsDNA), to a target site of interest (e.g., an immune cell). LNPs typically have a size of less than about 1000 nm in at least one dimension. In some aspects, the LNPs of the present disclosure have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

As used herein, the term “human T cell” or “T cell” refers to a T cell isolated from a donor, particularly a human donor. T cells, and cells derived therefrom, include isolated T cells that have not been passaged in culture, T cells that have been passaged and maintained under cell culture conditions without immortalization, and T cells that have been immortalized and can be maintained under cell culture conditions indefinitely.

As used herein, an antigen binding polypeptide or antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

As used herein, an “epitope” refers to a localized region of an antigen to which an antigen binding polypeptide or antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303; Meth Enzymol (1985) volumes 114 & 115, eds Wyckoff H W et al., U.S. Pub. No. 2004/0014194), Bricogne G (1993) Acta Crystallogr D Biol Crystallogr 49(Pt 1): 37-60, Bricogne G (1997) Meth Enzymol 276A: 361-423, ed Carter C W, and Roversi et al., (2000) Acta Crystallogr D Biol Crystallogr 56(Pt 10): 1316-1323 (X-ray diffraction crystallography studies); and Champe et al., (1995) J Biol Chem 270: 1388-1394 and Cunningham B C & Wells J A (1989) Science 244: 1081-1085 (mutagenesis mapping).

Specific binding can be represented by a “binding affinity.” Binding affinity refers to an intrinsic binding affinity that reflects a 1:1 interaction between members of a binding pair (e.g., an antigen binding polypeptide complex and an antigen). Binding affinity can be measured and/or expressed in several ways known in the art, including, but not limited to, equilibrium dissociation constant (K_{D}). K_{D} is calculated from the quotient of k_{off}/kₒₙ, where kₒₙ refers to the association rate constant of, e.g., an antigen binding polypeptide complex to an antigen, and k_{off} refers to the dissociation of, e.g., an antigen binding polypeptide complex from an antigen. The kₒₙ and k_{off} can be determined by techniques known to one of ordinary skill in the art, such as Octet® BLI, BIAcore® or KinExA.

Accordingly, in some aspects, an antigen binding polypeptide complex provided herein is an antibody or antigen binding fragment thereof. In some aspects, an antigen binding polypeptide provided herein is part of an antibody or antigen-binding fragment thereof. In some aspects, the antibody or antigen binding fragment thereof specifically binds to an antigen with an equilibrium dissociation constant (K_{D}) of from about 10 μM to about 1 pM.

### I. Targeted LNPs

The present disclosure provides targeted delivery systems comprising a payload-loaded LNP comprising a surface anchored targeting molecule (e.g., an antibody) that specifically binds to specific surface proteins (e.g., one, two, three, or more) on the surface of a particular cell type or tissue thereby directing the payload (e.g., a therapeutic mRNA, for example an mRNA encoding a vaccine or a CAR) to the targeted cell type or tissue. In some aspects, the delivery system targets at least two cell or tissue specific surface proteins. In some aspects, the cell or tissue targeted delivery system comprises a LNP comprising a surface anchored targeting molecule (e.g., an antibody) that specifically binds to at least one cell or tissue specific surface protein. Also provided is a cell or tissue targeted delivery system comprising an LNP comprising at least two surface anchored targeting molecules (e.g., two antibodies), wherein each cell- or tissue-specific targeting molecule specifically binds to at least one cell- or tissue-specific target protein (e.g., a first antibody targets a first target protein and a second antibody targets a second target protein). Also provided is a cell or tissue targeted delivery system comprising a set of LNPs comprising at least two LNPs, wherein the first LNP comprises a first cell- or tissue-specific targeting molecule (e.g., an antibody) that specifically binds to a first cell or tissue specific surface protein, and the second LNP comprises a second cell- or tissue-specific targeting molecule that specifically binds to a second cell- or tissue-specific surface protein. In some aspects, the LNP in a cell- or tissue-specific targeted delivery system of the present disclosure encapsulates a payload (e.g., an mRNA or a linear DNA fragment).

In some aspects, the present disclosure provides LNP delivery systems which are unconjugated, i.e., the LNP is not decorated with surface anchored targeting molecules (e.g., antibodies) that specifically binds to a specific surface protein in a target cell. In some aspects, the unconjugated LNP does not comprise a lipid with a derivatizable group (e.g., a maleimide group). In some aspects, the unconjugated LNP comprises a lipid with a derivatizable group (e.g., a maleimide). In other words, in some aspects the unconjugated LNP is an LNP specifically designed for delivery without the need of attaching a targeting molecule (e.g., an antibody) to the surface of the LNP, whereas in some aspects the unconjugated LNP is an intermediate used to produce a targeted delivery system comprising a LNP having a surface anchored targeting molecule (e.g., antibody) that specifically binds to a specific surface protein in a target cell.

It is to be understood that disclosures related to an unconjugated LNP, e.g., an LNP comprising Lipid 10 25%, DSPC 16%, cholesterol 56.5%, DMG-PEG2000 2.45%, and DSPE-PEG2000-maleimide 0.05%, also encompass the corresponding derivatized LNP, i.e., Lipid 10 25%, DSPC 16%, cholesterol 56.5%, DMG-PEG2000 2.45%, DSPE-PEG2000-maleimide-antibody 0.05% (e.g., Lipid 10 25%, DSPC 16%, cholesterol 56.5%, DMG-PEG2000 2.45%, DSPE-PEG2000-maleimide-antiCD3/antiCD28 0.05%), and unconjugated forms that do not include a chemically modified lipid to incorporate a targeting molecule onto the surface of the LNP. In the later case, the molar % of chemically modified lipid would be added to the molar % of PEG-lipid. Accordingly, for example, the disclosure of an unconjugated LNP comprising Lipid 10 25%, DSPC 16%, cholesterol 56.5%, DMG-PEG2000 2.45%, and DSPE-PEG2000-maleimide 0.05% LNP would encompass the corresponding unconjugated LNP comprising Lipid 10 25%, DSPC 16%, cholesterol 56.5%, DMG-PEG2000 2.5% (the amount of DMG-PEG2000 would be the amount of DMG-PEG2000 plus the amount of DSPE-PEG2000-maleimide present in the LNP for derivatization).

The present disclosure provides a targeted delivery system comprising a LNP comprising a surface anchored targeting molecule (e.g., antibody) that specifically binds to a specific surface protein in a target cell (e.g., a T cell, B cell, muscle cell, fibroblast, etc.) or tissue (e.g., a specific organ, a tumor, or the microenvironment of a tumor) and efficiently delivers a payload encapsulated in the LNP to the target cell or tissue.

Any combination of cells within the hematopoietic lineage can be targeted by the targeted delivery systems of the present disclosure. In some aspects, target cells selected from bone marrow cells, hematopoietic stem cells (HSCs), hematopoietic stem and progenitor cells (HSPCs), peripheral blood mononuclear cells (PBMCs), myeloid progenitor cells, lymphoid progenitor cells, T cells, B-cells, NKT cells, NK cells, dendritic cells, monocytes, granulocytes, erythrocytes, megakaryocytes, mast cells, basophils, eosinophils, neutrophils, macrophages, erythroid progenitor cells (e.g., HUDEP cells), megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic 3-islet cells, liver cells, muscle cells, skeletal muscle cells, cardiac muscle cells, hepatic cells, fat cells, intestinal cells, cells of the colon, and cells of the stomach.

Non-limiting examples of various applications of the targeted delivery systems of the present disclosure (e.g., for targeting neurons, cells of the pancreas, hematopoietic stem cells and multipotent progenitors, etc.) are described next. For example, Hematopoietic stem cells and multipotent progenitors can be targeted for gene editing (e.g., insertion) in vivo. As another example, pancreatic cells (e.g., β islet cells) can be targeted, e.g., to treat pancreatic cancer, to treat diabetes, etc. As another example, somatic cells in the brain such as neurons can be targeted (e.g., to treat indications such as Huntington's disease, Parkinson's (e.g., LRRK2 mutations), and ALS (e.g., SOD1 mutations)). As another example, endothelial cells and cells of the hematopoietic system (e.g., megakaryocytes and/or any progenitor cell upstream of a megakaryocyte such as a megakaryocyte-erythroid progenitor cell (MEP), a common myeloid progenitor cell (CMP), a multipotent progenitor cell (MPP), a hematopoietic stem cells (HSC), a short term HSC (ST-HSC), an IT-HSC, a long term HSC (LT-HSC) can be targeted with a subject delivery molecule to treat Von Willebrand's disease. For example, a cell (e.g., an endothelial cell, a megakaryocyte and/or any progenitor cell upstream of a megakaryocyte such as an MEP, a CMP, an MPP, an HSC such as an ST-HSC, an IT-HSC, and/or an LT-HSC) harboring a mutation in the gene encoding von Willebrand factor (VWF) can be targeted (in vitro, ex vivo, in vivo) in order to introduce an active protein (e.g., via delivery of a functional VWF protein and/or a nucleic acid encoding a functional VWF protein) and/or in order to edit the mutated gene, e.g., by introducing a replacement sequence (e.g., via delivery of a gene editing tool and delivery of a DNA donor template). In some of the above cases (e.g., in cases related to treating Von Willebrand's disease, in cases related to targeting a cell harboring a mutation in the gene encoding VWF), a subject targeting ligand provides for targeted binding to E-selectin. As another example, a cell of a stem cell lineage (e.g., a stem and/or progenitor cell of the hematopoietic lineage, e.g., a GMP, MEP, CMP, MLP, MPP, and/or an HSC) can be targeted with a subject delivery molecule (or subject viral or non-viral delivery vehicle) in order to increase expression of stem cell factor (SCF) in the cell, which can therefore drive proliferation of the targeted cell. For example, a subject delivery molecule can be used to deliver SCF and/or a nucleic acid (DNA or mRNA) encoding SCF to the targeted cell. Methods and compositions of this disclosure can be used to treat any number of diseases, including any disease that is linked to a known causative mutation, e.g., a mutation in the genome. For example, methods and compositions of this disclosure can be used to treat sickle cell disease, R thalassemia, HIV, myelodysplasia syndromes, JAK2-mediated polycythemia vera, JAK2-mediated primary myelofibrosis, JAK2-mediated leukemia, and various hematological disorders. As additional non-limiting examples, the methods and compositions of this disclosure can also be used for B-cell antibody generation, immunotherapies (e.g., delivery of a checkpoint blocking reagent), and stem cell differentiation applications.

The present disclosure also provides a T cell targeted delivery system comprising (a) a LNP comprising a surface anchored T cell targeting molecule (e.g., an antibody) that specifically binds to a T cell surface protein (e.g., CD3), and (b) a costimulator of T cell activation. In some aspects, the costimulator of T cell activation is a CD28 agonist. In some aspects, the CD28 agonist is an antibody that specifically binds CD28 or an antigen-binding portion thereof. In some aspects, the CD28 agonist is selected from the group consisting of an antibody that specifically binds CD28, a CD28 ligand, an aptamer, a peptide, a small molecule, or, a combination thereof. In some aspects, the CD28 ligand is B7-1 (CD80), B7-2 (CD86), or a combination thereof. In some aspects, the aptamer is CD28Apt7-dimer (CD28Apt7 having the forward polynucleotide sequence of SEQ ID NO: 140, and the reverse polynucleotide sequence of SEQ ID NO: 141. In some aspects, the costimulator of T cell activation is an agonist of ICOS, B7, CD226, CRTAM, 41-BB, OX40, CD27, GITR, HVEM, CD40, BAFFR, or BAFF. See Chen & Flies (2013) Nat. Rev. Immunol. 13(3):227-242, which is herein incorporated by reference in its entirety.

The present disclosure provides a T cell targeted delivery system comprising a payload-loaded LNP comprising a surface anchored T cell targeting molecule that specifically binds to CD3, wherein the delivery system targets a payload to the T cell.

The present disclosure provides a T cell targeted delivery system comprising a payload-loaded LNP comprising a surface anchored T cell targeting molecule (e.g., an antibody) that specifically binds to at least two T cell specific surface proteins (e.g., CD3 and CD28), wherein the delivery system targets the least two T cell specific surface proteins. In some aspects, the present invention provides a T cell targeted delivery system comprising a LNP comprising a surface anchored T cell targeting molecule (e.g., an antibody) that specifically binds to at least one T cell specific surface protein selected from CD3 and which is used in combination with an anti-CD28 to activate the T cell and facilitate delivery of the payload within the LNP. Also provided is a T cell targeted delivery system comprising an LNP comprising at least two surface anchored T cell targeting molecules (e.g., two antibodies), wherein each T cell targeting molecule specifically binds to at least one T cell specific surface protein (e.g., a first antibody targets CD3 and a second antibody targets CD28), and wherein the delivery system targets the least two T cell specific surface proteins (e.g., CD3 and CD28). Also provided is a T cell targeted delivery system comprising a set of LNPs comprising at least two LNPs, wherein the first LNP comprises a first T cell targeting molecule (e.g., an antibody) that specifically binds to a first T cell specific surface protein, and the second LNP comprises a second T cell targeting molecule that specifically binds to a second T cell specific surface protein. In some aspects, the LNP in a T cell targeted delivery system of the present disclosure encapsulates a payload (e.g., an mRNA or a linear DNA fragment).

There is a wide range of therapeutic payloads that can be encapsulated in LNPs, including for example, small molecules, proteins, nucleic acids, and diagnostic agents. In some aspects, the payload can comprise a nucleic acid such as an mRNA, a small interfering RNA (siRNA), a vector, etc. For example, the payload can comprise a polynucleotide encoding an enzyme, e.g., for gene replacement therapy, or an enzyme. In some aspects, the payload can comprise a component of a gene editing system, e.g., a polynucleotide encoding a Cas enzyme or a Cas enzyme, or a gRNA. In some aspects, the payload can comprise an mRNA encoding, e.g., a vaccine, for example, a SARS-CoV2 vaccine (e.g., mRNA-1273 or BNR162b2). In some aspects, the payload can comprise a small molecule, e.g., a chemotherapy agent. In some aspects, the payload can comprise a chimeric antigen receptor (CAR). In some aspects, the payload can comprise a T cell receptor (TCR). In some aspects, the payload can comprise a hormone. In some aspects, the payload can comprise a growth factor. In some aspects, the payload can comprise an anti-inflammatory protein. Payloads that can be carried by the LNP delivery systems of the present disclosure are disclosed in detail in Section I-B, below.

The LNP delivery systems disclosed herein can target any cell, tissue, or cellular compartment as long as the appropriate surface anchored targeting molecule or set thereof are capable of specifically binding to one or more target molecules (e.g., receptors) on the target cell, tissue, or cellular compartment. In some aspects, the LNP delivery systems of the present disclosure can target a human cell, e.g., a T cell. In some aspects, the LNP delivery system of the present disclosure can target an organ, e.g., liver. In some aspects, the LNP delivery system of the present disclosure can target a tissue, e.g., muscle tissue. In some aspects, the LNP delivery system of the present disclosure can target a tumor. In some aspects, the LNP delivery system of the present disclosure can target bacteria. In some aspects, the LNP delivery system of the present disclosure can target a virus.

In some aspects, the present disclosure provides T cell targeted delivery systems comprising, e.g., (a) a single LNP population with bispecific antibodies conjugated to their surface, wherein the bispecific antibodies target two different T cell antigens (e.g., two T cell specific surface proteins), (b) a single LNP population with monospecific antibodies (either monovalent, bivalent, or polyvalent) conjugated to their surface, wherein each monospecific antibody target a different T cell antigen (e.g., two T cell specific surface proteins), or (c) two LNP populations wherein each population comprises a LNP with a monospecific antibody (either monovalent, bivalent, or polyvalent) conjugated to their surface, wherein the monospecific antibody of each LNP population targets a different T cell antigen (e.g., one of two T cell specific surface proteins).

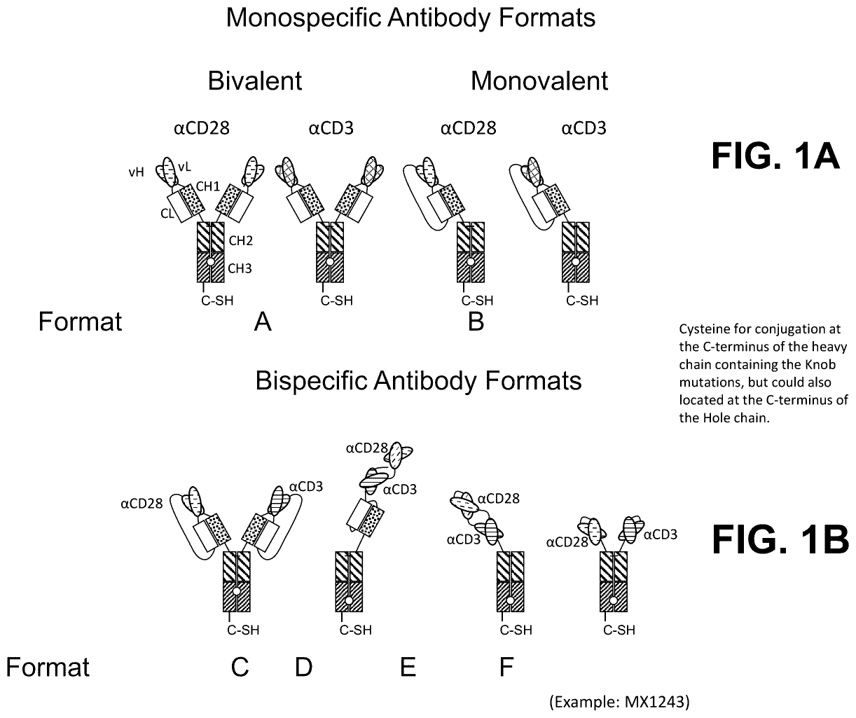

An exemplary architecture of a LNP of the present disclosure having a bispecific antibody targeting CD3 and CD28 is shown in FIG. 7A. Other antibody formats suitable for conjugation to the surface of LNP of the present disclosure are shown in FIG. 1A and FIG. 1B. The aCD3 (anti-CD3) and aCD28 (anti-CD28) labels of each of the formats shown in FIG. 1A and FIG. 1B are exemplary, and can be supplemented with molecule (e.g., an antibody, ligand, polypeptide, or interacting protein) capable of binding at least one additional T cell specific surface protein selected from the group consisting of CD2, CD3, CD4, CD28, CD8, CD5, CD7, CD40L (CD154), LFA-1 (CD11a/CD18), CD45, CTLA-4 (CD152), ICAM (CD50), 4-1BB, and combinations thereof. In some aspects, the cell is a B-cell, and therefore B-cell surface proteins are selected, for example CD74, CD19 or CD40L.

In some aspects, a T cell targeted delivery system of the present disclosure comprises a LNP having a Format C bispecific antibody, as shown in FIG. 1B, conjugated to its surface. In some aspects, a T cell targeted delivery system of the present disclosure comprises a LNP having a Format D bispecific antibody, as shown in FIG. 1B, conjugated to its surface. In some aspects, a T cell targeted delivery system of the present disclosure comprises a LNP having a Format E bispecific antibody, as shown in FIG. 1B, conjugated to its surface. In some aspects, a T cell targeted delivery system of the present disclosure comprises a LNP having a Format F bispecific antibody, as shown in FIG. 1B, conjugated to its surface.

In some aspects, a T cell targeted delivery system of the present disclosure comprises a LNP having two Format A monospecific antibodies, as shown in FIG. 1A, conjugated to its surface, wherein each Format A monospecific antibody targets a different T cell specific surface protein. In some aspects, a T cell targeted delivery system of the present disclosure comprises a LNP having two Format B monospecific antibodies, as shown in FIG. 1A, conjugated to its surface, wherein each Format B monospecific antibody targets a different T cell specific surface protein. In some aspects, a T cell targeted delivery system of the present disclosure comprises a LNP having one Format A monospecific antibody and one Format B monospecific antibody, as shown in FIG. 1A, conjugated to its surface, wherein the Format A and Format B monospecific antibodies target different T cell specific surface proteins.

In some aspects, a T cell targeted delivery system of the present disclosure comprises two LNPs, wherein each LNP has a Format A monospecific antibody, as shown in FIG. 1A, conjugated to its surface, and wherein each Format A monospecific antibody targets a different T cell specific surface protein. In some aspects, a T cell targeted delivery system of the present disclosure comprises two LNPs, wherein each LNP has a Format B monospecific antibody, as shown in FIG. 1A, conjugated to its surface, and wherein each Format B monospecific antibody targets a different T cell specific surface protein. In some aspects, a T cell targeted delivery system of the present disclosure comprises two LNPs, a LNP having one Format A monospecific antibody and the other LNP having one Format B monospecific antibody, as shown in FIG. 1A, conjugated to its surface, wherein the Format A and Format B monospecific antibodies target different T cell specific surface proteins.

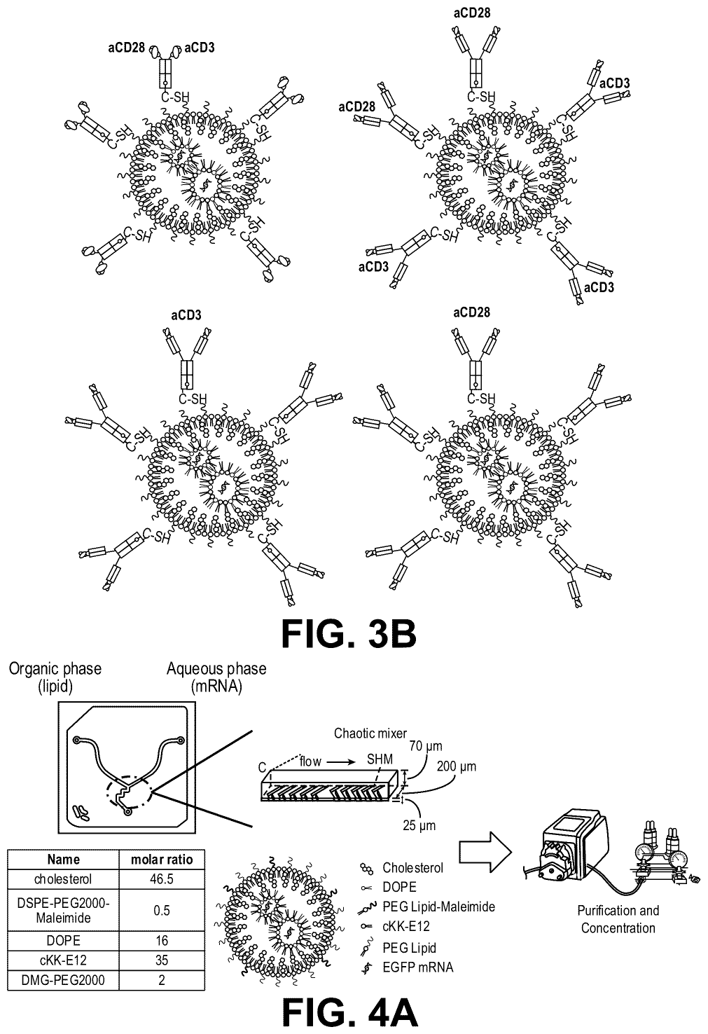

FIG. 3B shows a schematic representation of exemplary delivery systems of the present disclosure. The top left graphic of FIG. 3B represents a LNP delivery system of the present disclosure comprising bispecific antibodies attached to its surface. Each of those bispecific antibodies may bind, e.g., to CD3 and to CD28. The top right graphic of FIG. 3B represents a LNP delivery system of the present disclosure comprising two populations of monospecific antibodies, one specific for CD3 and the other one specific for CD28. The bottom graphics of FIG. 3B represent two populations of LNPs, each population comprising a single type of monospecific antibodies, e.g., anti CD3 and anti CD38. A delivery system of the present disclosure may comprise combination of these two populations of LNPs.

In some aspects, targeting of a LNP of the present disclosure can be achieved, e.g., by a Fab fragment or an MSTAR antibody. In some aspects, the MSTAR antibody comprises a single chain CHCL. In some aspects, the MSTAR antibody does not comprise a single chain CHCL. In some aspects, the MSTAR antibody is monospecific. In some aspects, the MSTAR antibody is bispecific. In some aspects, the MSTAR antibody is trispecific. In some aspects, the MSTAR antibody is multispecific.

In some aspects, the lipid delivery systems of the present disclosure is a nanocarrier, e.g., a LNP. In some aspects, the nanocarrier is liposome or a micelle. Therapeutic liposomes are 50-200 nm in size. Micelles are colloidal dispersion with a particle size between 5-100 nm. In some aspects, the nanocarrier is an extracellular vesicle. In some aspects, the extracellular vesicle is an exosome (30-200 nm) or a microvesicle (100-1000 nm).

In some aspects, the T cell targeted delivery system comprises a T cell targeting molecule (e.g., an antibody) or set thereof that specifically binds to at least two T cell specific surface proteins, e.g., CD3 and CD28. In some aspects, the T cell targeted delivery system targets two, three, four, five, or six T cell specific surface proteins. In some aspects, the T cell targeted delivery system targets two, three, four, five, or six T cell specific surface proteins, wherein the T cell targeting molecule or set thereof specifically binds to CD3 and/or CD28, and wherein the binding activates CD3 and CD28. In some aspects, the T cell targeting molecule or set thereof specifically binds to T cell specific surface proteins selected from the group consisting of CD2, CD3, CD4, CD28, CD8 CD5, CD7, CD40L (CD154), LFA-1 (CD11a/CD18), CD45, CTLA-4 (CD152), ICAM (CD50), CD19, CD20, BCMA, CD22, PSMA, FAP, 4-1BB, OX40 and CA IX, wherein at least one T cell targeting molecule or set thereof specifically binds to CD3 and/or CD28, and wherein the binding activates CD3 and CD28.

In some aspects of the T cell targeted delivery systems disclosed herein, the at least two T cell specific surface proteins comprise CD3 and CD28, CD2 and CD3, CD2 and CD4, CD3 and CD4, CD2 and CD28, CD4 and CD28, CD3 and OX40, or CD3 and 4-1BB.

In some aspects of the T cell targeted delivery systems disclosed herein the at least two T cell specific surface proteins consists of CD3 and CD28, CD2 and CD3, CD2 and CD4, CD3 and CD4, CD2 and CD28, CD4 and CD28, CD3 and OX40, CD3 and 4-1BB. In some aspects, the present disclosure provides a B-cell targeted delivery system wherein at least two B-cell specific proteins are targeted, e.g., CD74 and CD40L, or CD19 and CD40L.

In some aspects, the T cell targeted delivery system comprises a T cell targeting molecule (e.g., an antibody) and a second binding molecule that is a natural binding partner of a T cell surface protein. In some aspects, the second binding molecule on the T cell targeted delivery system is CD58 and binds to CD2 on the T cell surface. In some aspects, the second binding molecule on the T cell targeted delivery system is CD80 and binds to CD28 on the T cell surface. In some aspects, the second binding molecule on the T cell targeted delivery system is CD86 and binds to CD28 on the T cell surface. In some aspects, the second binding molecule on the T cell targeted delivery system is CD80 and binds to CTLA-4 on the T cell surface. In some aspects, the second binding molecule on the T cell targeted delivery system is CD86 and binds to CTLA-4 on the T cell surface. In some aspects, the second binding molecule on the T cell targeted delivery system is ICOSL and binds to ICOS on the T cell surface. In some aspects, the second binding molecule on the T cell targeted delivery system is MHC II and binds to the T cell receptor (TCR) on the T cell surface. In some aspects, the second binding molecule on the T cell targeted delivery system is PD-L1 and binds to CD80 on the T cell surface. In some aspects, the second binding molecule on the T cell targeted delivery system is PD-L1 and binds to PD-1 on the T cell surface. In some aspects, the second binding molecule on the T cell targeted delivery system is PD-L2 and binds to PD-1 on the T cell surface. In some aspects, the second binding molecule on the T cell targeted delivery system is 4-1BBL and binds to 4-1BB on the T cell surface. In some aspects, the second binding molecule on the T cell targeted delivery system is OX40L and binds to OX40 on the T cell surface. In some aspects, the second binding molecule on the T cell targeted delivery system is CD40 and binds to CD154 on the T cell surface. In some aspects, the second binding molecule on the T cell targeted delivery system is ICAM-1 and binds to LFA-1 on the T cell surface.

As used herein, “CD3” refers to the human Cluster of Differentiation 3 protein that is part of the T cell co-receptor protein complex and is composed of four distinct chains. The complex contains a CD3γ (gamma) chain (human CD37 chain; Uniprot P09693), a CD3δ (delta) chain (human CD3δ; Uniprot P04234), two CD3ε (epsilon) chains (human CD3ε; Uniprot P07766), and a CD3ζ (zeta) chain (human CD3ζ; Uniprot P20963). These chains associate with a molecule known as the T cell receptor (TCR) and generate an activation signal in T cells. The TCR and CD3 molecules together comprise the TCR complex.

As used herein “CD28” refers to the human Cluster of Differentiation 28 protein (Uniprot P10747), which is one of the proteins expressed on T cells that provide co-stimulatory signals required for T cell activation and survival. T cell stimulation through CD28 in addition to the T cell receptor (TCR) can provide a potent signal for the production of various interleukins (IL-6 in particular). CD28 is the receptor for CD80 (B7.1) and CD86 (B7.2) proteins.

As used herein “CD2” refers to the human Cluster of Differentiation 2 protein (Uniprot P06729; Q53F96). CD2 is a cell adhesion molecule found on the surface of T cells and natural killer (NK) cells. It has also been called T cell surface antigen T11/Leu-5, LFA-2, LFA-3 receptor, erythrocyte receptor and rosette receptor. CD2 interacts with CD2BP2, Lck and PSTPIP1.

As used herein “CD4” refers to the human Cluster of Differentiation 4 protein (Uniprot P01730). CD4 is a glycoprotein that serves as a co-receptor for the T cell receptor (TCR). CD4 is found on the surface of immune cells, e.g., T helper cells, monocytes, macrophages, or dendritic cells.

As used herein “CD8” refers to the human Cluster of Differentiation 8 protein (Uniprot P01732). CD8 is a glycoprotein co-receptor for MHC class I molecule:peptide complex. CD8 is found on the surface of cytotoxic T cells, NK cells, memory T cells, monocytes, and dendritic cells.

As used herein “ICOS” refers to the human inducible T cell co-stimulator (Uniprot Q9Y6W8). ICOS (CD278) is expressed on activated T cells.

As used herein “4-1BB” refers to CD137, a member of the tumor necrosis factor (TNF) receptor family, is a type 1 transmembrane protein, expressed on surfaces of leukocytes and non-immune cells. Its alternative names are tumor necrosis factor receptor superfamily member 9 (TNFRSF9), CDw137, and ILA (induced by lymphocyte activation). See Uniprot Q07011.

### I.A Lipid Nanoparticles (LNP)

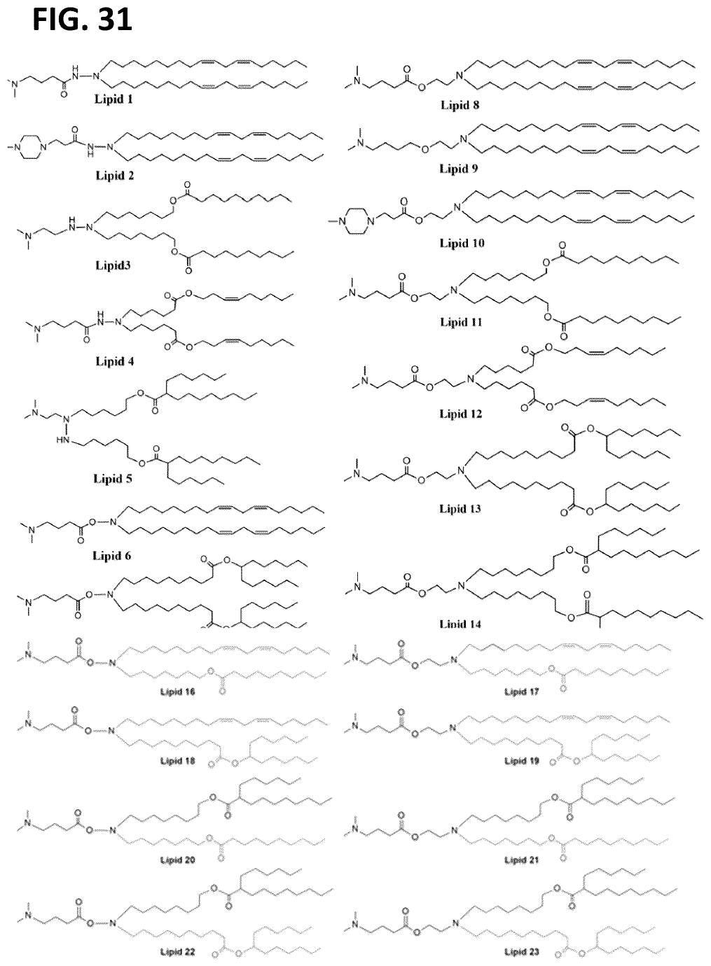

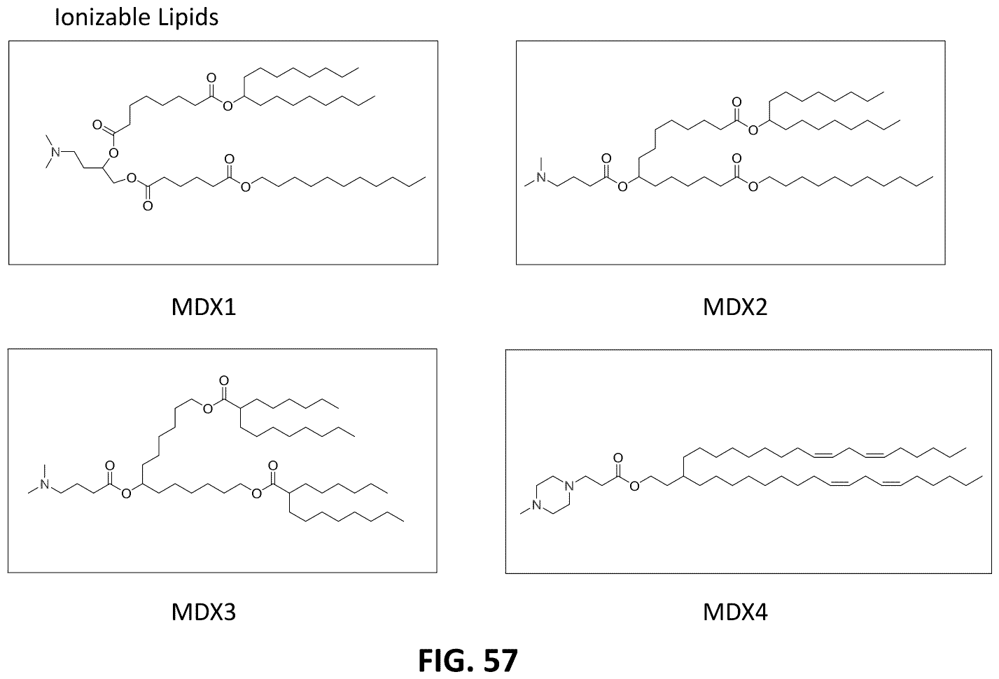

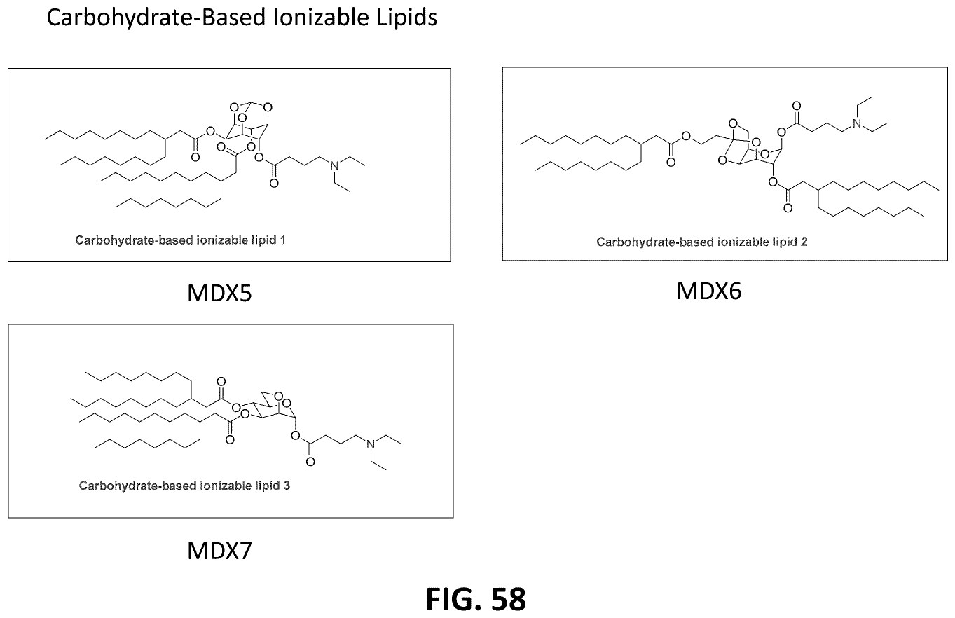

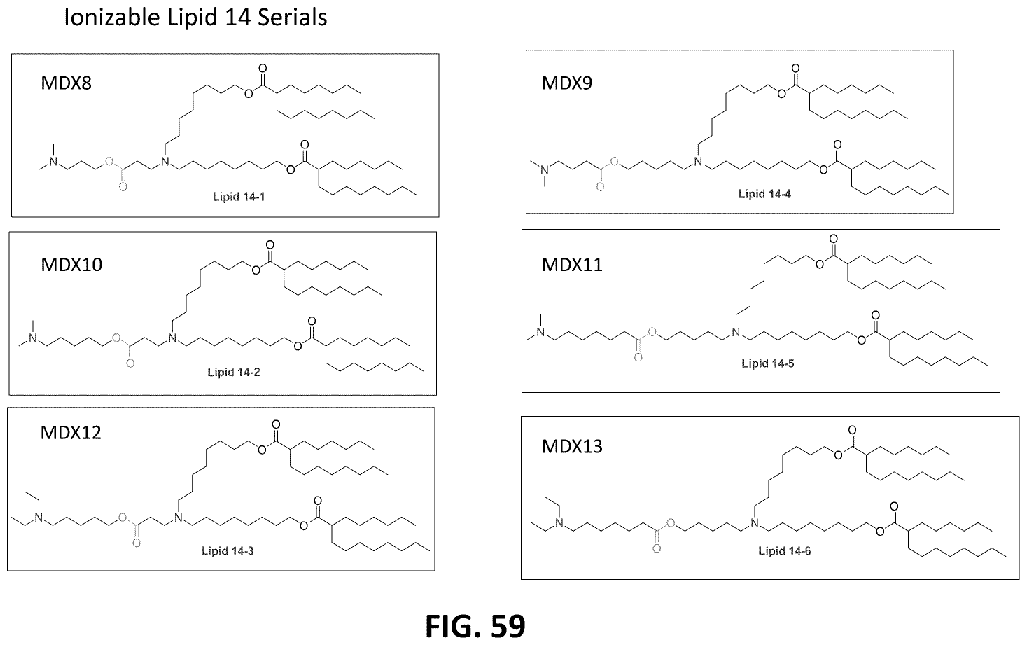

In some aspects, the LNP in the T cell targeted delivery systems the present disclosure comprises (a) a cationic or ionizable cationic lipid or lipidoid (e.g., cKK-E12, ALC-0315, SM-102, YK-009, MC3, KC2, A6, OF-02, A18-Iso5-2DC18, 98N12-5, 9A1p9, C12-200, 7C1, G0-C14, L319, 304O13, OF-Deg-Lin, 306-O12B, 306Oi10, FTT5, Lipid 8 (shown in FIG. 31), Lipid 10 (shown in FIG. 31), Trialkyl Lipid 10, or any one of MDX1-MDX13 (shown in FIGS. 57-59)); (b) a structural lipid (e.g., cholesterol or beta-cholesterol); (c) a helper lipid (e.g., a phospholipid such as DSPE, DSPC, DOPC or DOPE); and, (d) a stabilizing lipid (e.g., a PEG lipid such as DMG-PEG1000, DMG-PEG2000, DMG-PEG1000).

### I.A.i Cationic or Ionizable Cationic Lipids or Lipidoids

The terms “ionizable cationic lipid” and “ilipid” are used interchangeably and refer to any of a number of lipid species that carry a net positive charge at a selected pH, such as physiological pH 4 and a neutral charge at other pHs such as physiological pH 7. Ionizable cationic lipids can be used as a component of a LNP to facilitate or enhance the delivery and release of a nucleic acid, e.g., an RNA, to one or more target cells (e.g., by permeating or fusing with the lipid membranes of such target cells). As used herein, the term “lipidoid” refers to a type of synthetic lipid-like molecule that is used for intracellular delivery of various bioactive cargos and it is composed of lipid-like structures that can self-assemble into nanoparticles for efficient delivery of therapeutic agents. In the context of the present disclosure, the terms “ionizable cationic lipid,” “ilipid,” and “lipidoid” are used interchangeably.

In some aspects, the ionizable cationic lipid or lipidoid is cKK-E12 (3,6-bis(4-(bis(2-hydroxydodecyl)amino)butyl)piperazine-2,5-dione). In some aspects, the ionizable cationic lipid or lipidoid is selected from the group consisting of cKK-E12, ALC-0315, SM-102, YK-009, MC3, KC2, A6, OF-02, A18-Iso5-2DC18, 98N₁₂−5, 9A1p9, C12-200, 7C1, G0-C14, L319, 304O₁₃, OF-Deg-Lin, 306-O12B, 306Oᵢ₁₀, FTT5, Lipid 8 (see FIG. 31), Lipid 10 (see FIG. 31), Trialkyl Lipid 10, and combinations thereof.

cKK-E12 is an ionizable cationic lipomer that has been used in combination with other lipids in the formation of LNPs for the delivery of mRNA. ALC-0315 is an ionizable cationic lipid that has been used to form LNPs for delivery of RNA. ALC-0315 is one of the components in the BNT162b2 vaccine against SARS-CoV-2 in addition to ALC-0159, DSPC, and cholesterol. SM-102 is a synthetic amino lipid that is used in combination with other lipids to form LNPs. These are used for the delivery of mRNA-based vaccines, and in particular SM-102 forms part of the drug delivery system for the Moderna COVID-19 vaccine.

In some aspects, the cationic or ionizable cationic lipid or lipidoid, used as a component of a LNP described herein, is selected from the group consisting of MDX1-MDX13 (see FIGS. 57-59), and combinations thereof. In some aspects, the LNP comprises MDX1. In some aspects, the LNP comprises MDX2. In some aspects, the LNP comprises MDX3. In some aspects, the LNP comprises MDX4. In some aspects, the LNP comprises MDX5. In some aspects, the LNP comprises MDX6. In some aspects, the LNP comprises MDX7. In some aspects, the LNP comprises MDX8. In some aspects, the LNP comprises MDX9. In some aspects, the LNP comprises MDX10. In some aspects, the LNP comprises MDX11. In some aspects, the LNP comprises MDX12. In some aspects, the LNP comprises MDX13. MDX1-MDX13 contain a combination of, for example, SM-102 and KC2, and can be made by routine synthetic methods known in the art.

In some aspects, an ionizable cationic lipid comprises one or more cleavable functional groups (e.g., a disulfide) that allow, for example, a hydrophilic functional head-group to dissociate from a lipophilic functional tail-group of the compound (e.g., upon exposure to oxidative, reducing or acidic conditions), thereby facilitating a phase transition in the lipid bilayer of the one or more target cells. In some aspects, an ionizable cationic lipid is a lipid as represented by formula 1 or as listed in Tables 1 or 2 of U.S. Pat. No. 9,708,628, the content of which is herein incorporated by reference in its entirety. In some aspects, an ionizable cationic lipid is as described in pages 7-13 of U.S. Pat. No. 9,765,022 or as represented by formula 1 of U.S. Pat. No. 9,765,022, the content of which is herein incorporated by reference in its entirety. In some aspects, an ionizable cationic lipid is described in pages 12-24 of WO2019152848A1 or as represented by formula 1 of International Patent Application WO2019152848A1, the contents of which are herein incorporated by reference in their entireties.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In some aspects, the cationic lipid comprises a zwitterionic lipid that assumes a positive charge on pH decrease.

In some aspects, the cationic lipid is DOTAP or DOTMA. In some aspects, the cationic lipid comprises any of a number of lipid species that carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, commercial preparations of cationic lipids are available which can be used in the LNP of the present disclosure. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(14(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: 20 DODAP, DODMA, DMDMA, 1,2-dilinoleyloxy-N,N-dimethylaminopropane (DLinDMA), and 1,2-dilinolenyloxy-N,N-dimethylaminopropane (DLenDMA).

In some aspects, the cationic lipid is an amino lipid. Suitable amino lipids useful in the LNP of the present disclosure include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), I-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt 30 (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

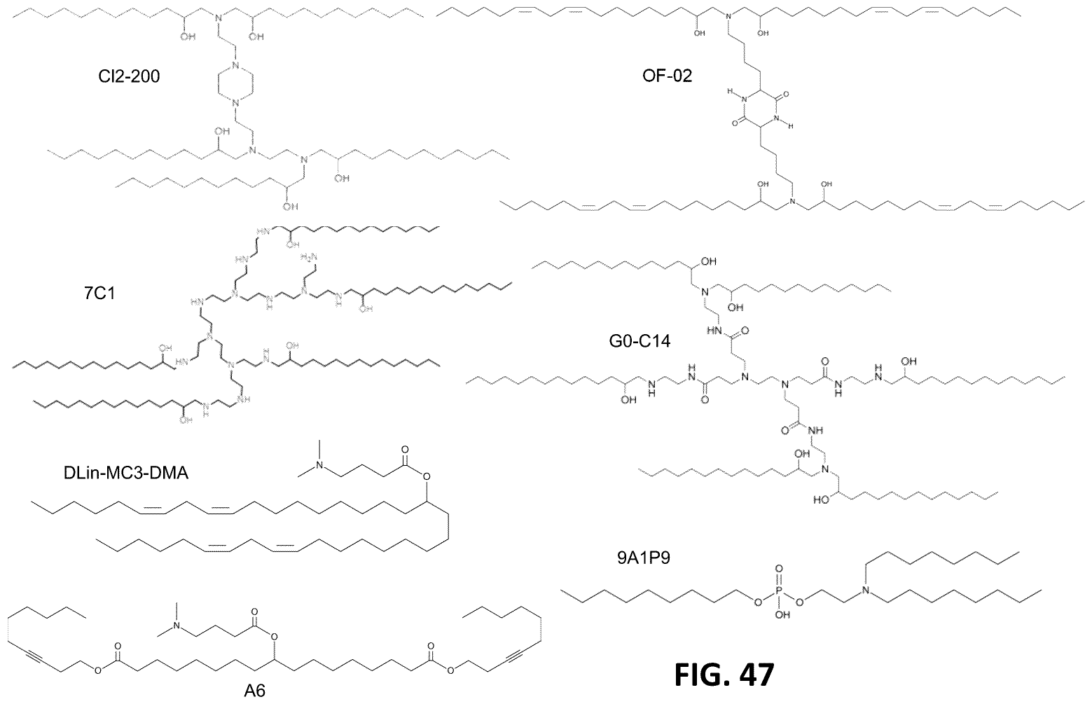

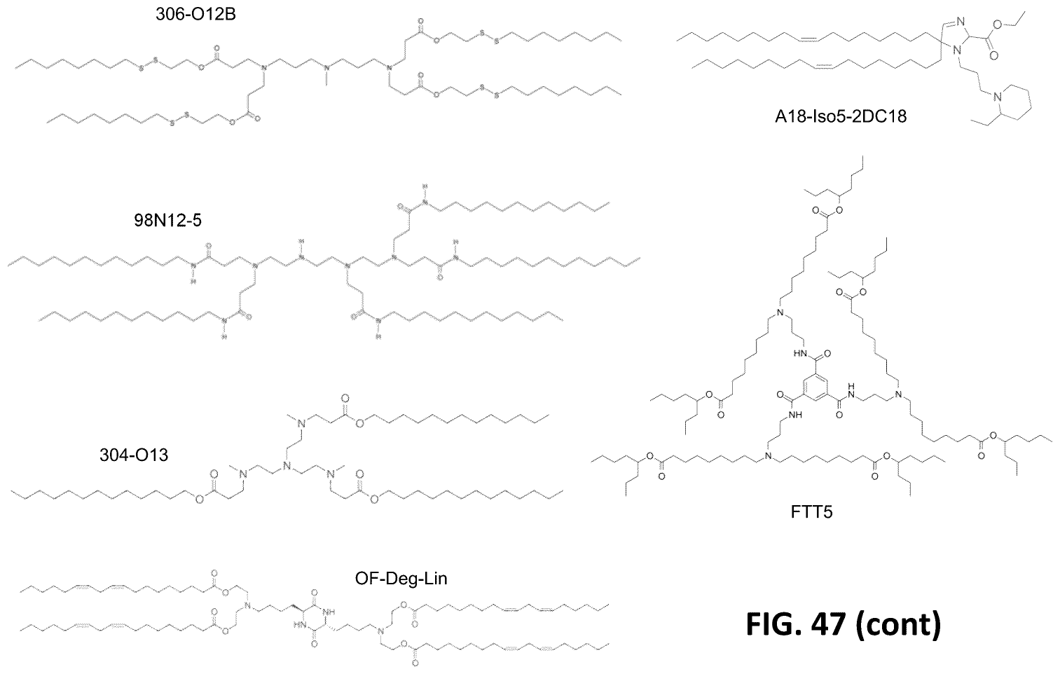

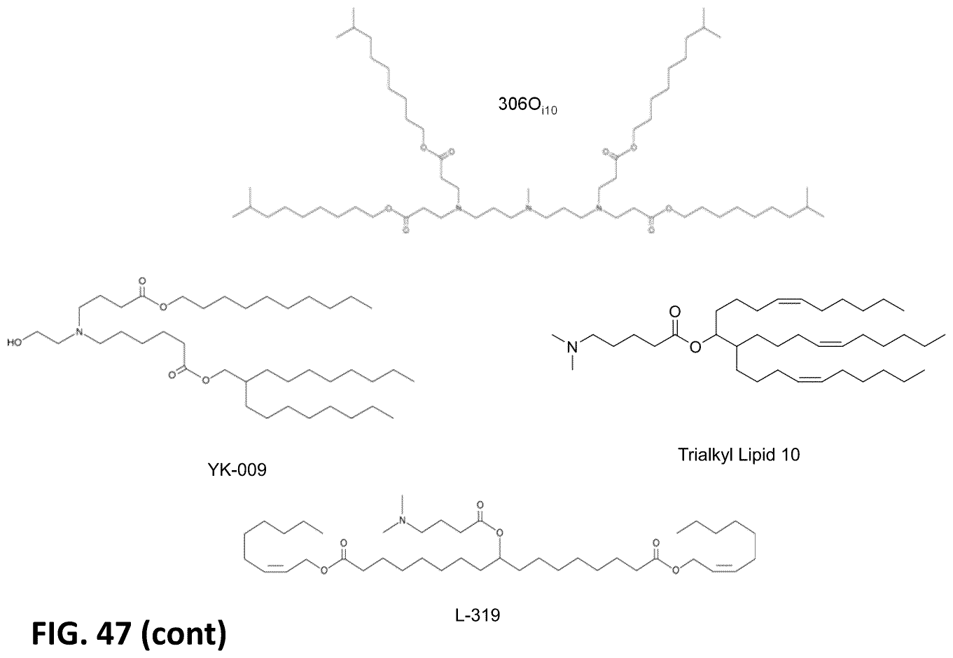

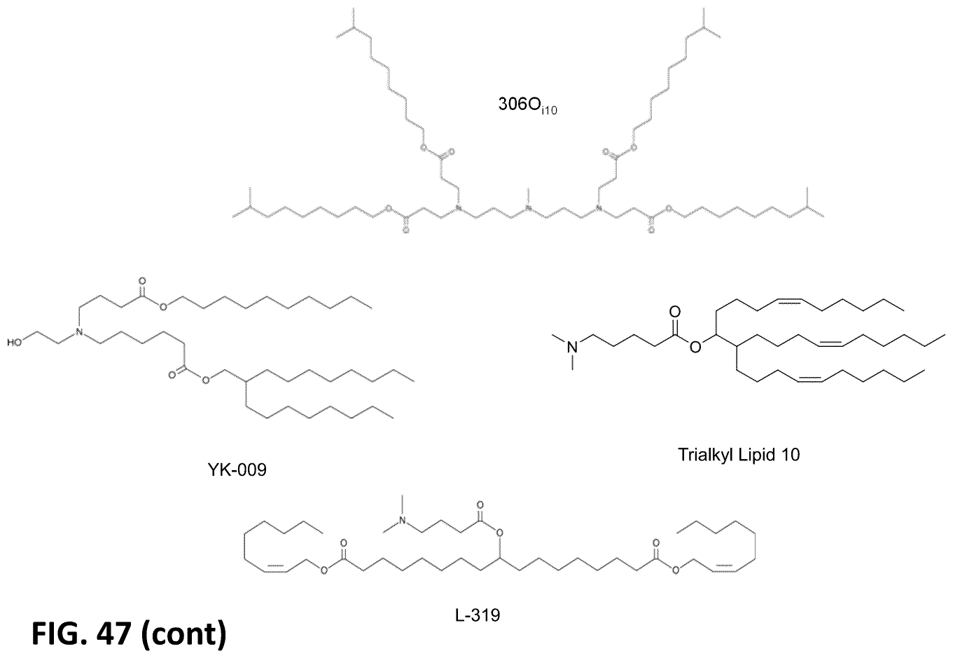

The structures of 7C1 (available, e.g., from Xcess Biosciences; Catalog No. M33911-C); DLin-MC3-DMA (available, e.g., from Cayman Chemical; Item No. 34364); A6 (available, e.g., from Cayman Chemical; Item No. 35052); C12-200 (available, e.g., from Cordon Pharma; Item No. LP-04-425); OF-02 (available, e.g., from Cayman Chemical; Item No. 37652); 98N12-5 (available, e.g., from Cayman Chemical; Item No. 37651); 9A1P9 (available, e.g., from Cayman Chemical; Item No. 37276); G0-C14 (available, e.g., from MedChemExpress; Cat. No. HY-152229); L-319 (available, e.g., from Cayman Chemical; Item No. 35051); 304-O13 (available, e.g., from Corden Pharma; Item No. LP-R4-520); OF-Deg-Lin (available, e.g., from Corden Pharma; Item No. LP-R4-516); 306-O12B (available, e.g., from Cayman Chemical; Item No. 37549); 306Oᵢ₁₀ (available, e.g., from Cayman Chemical; Item No. 36698); FTT5 (available, e.g., from MedChemExpress; Cat. No. HY-145793); YK-009 (available, e.g., from Cayman Chemical; Cat. No. 38282) and A18-Iso5-2DC18 (available, e.g., from Corden Pharma; Cat. No. LP-R4-517) are shown in FIG. 47.

As used herein, the term “Lipid 10” refers to 2-(di(9Z,12Z)-octadeca-9,12-dien-1-yl)amino)ethyl 3-(4-methylpiperazin-1-yl)propanoate (CAS: 2430034-02-7) as shown, e.g., in FIG. 1 of Ramishetti et al. (2020) Adv. Mater. 32: 1906128. The structure of Lipid 10 is shown in FIG. 31.

Lipid 10 is available, for example, from BroadPharm (Cat. No. BP-40632), MedChemExpress (Cat. No.: HY-150115), Echelon Biosciences (Cat. No. N-1110), or Cayman Chemical (Cat. No. 38705). See, e.g., Naidu et al. Adv. Sci. 10.19 (2023): 2301929; Ramishetti et al. Adv. Mat. 32.12 (2020): 1906128; and U.S. Pat. No. 11,851,389B2 (U.S. national phase of WO2018087753), which are herein incorporated by reference in their entireties. Lipid 10 is also known as EA-PIP. See, e.g., Granot-Matok et al. Theranostics 13.11 (2023): 3497.

As used herein, the term “Trialkyl Lipid 10” refers to ((6Z,16Z)-12-((Z)-dec-4-en-1-yl)docosa-6,16-dien-11-yl 5-(dimethylamino)pentanoate). See WO2013/126803, wherein Trialkyl Lipid 10 is described as Compound 13, and U.S. Pat. Nos. 11,395,854, 10,561,732, and 9,352,042, which are herein incorporated by reference in their entireties. The structure of Trialkyl Lipid 10 is shown in FIG. 47.

In some aspects, the cationic or ilipid component in a LNP of the present disclosure comprises an ionizable cationic lipid, an ionizable cationic amino lipid, an ionizable cationic lipidoid, a polyamine branched-chain lipidoid, a lipid catechol, an ionizable dendrimer, a branched-chain ionizable cationic lipidoid, an ionizable cationic trialkyl lipid, a biodegradable alkyne lipid, an ionizable cationic SSPalm, an ionizable cationic self-degradable SSPalm, a multi-tail ionizable cationic phospholipid or a combination thereof.

Cationic ionizable that can be used in the delivery systems of the present disclosure comprise 1,2(R)-Dioleyloxy-3-dimethylamino-propane (Cayman Chemical Item 8004302; CAS No. 666234-78-2, also known as R-DODMA), Lipid R6 (Cayman Chemical Item No. 39130), 3060i9-cis2 (Cayman Chemical Item No. 39557), Lipid 16 (Cayman Chemical Item No. 38861), Lipid AX4 (Cayman Chemical Item No. 39070, CAS No. 2735814-23-8), RM 137-15 (Cayman Chemical Item No. 38918), C12-113 (Cayman Chemical Item No. 39335; CAS No. 1220890-27-6), C12-SPM (Cayman Chemical Item No. 38784, CAS No. 2055647-81-7, also known as C12-spermine), Lipid 10 (Cayman Chemical Item No. 38705, CAS No. 2430034-02-7, also known as EA-PIP), RM 133-3 (Cayman Chemical Item No. 38917, CAS No. 2941228-90-4), AA-T3A-C12 (Cayman Chemical Item No. 38648), Lipid 23 (Cayman Chemical Item No. 38862), OC2-K3-E10 (Cayman Chemical Item No. 38243, also known as I-28), CL4F8-6 (Cayman Chemical Item No. 38802, CAS No. 2766493-12-1), Lipid Catechol (Cayman Chemical Item No. 38665), 1014 (Cayman Chemical Item No. 38150), Lipid III-45 (Cayman Chemical Item No. 39243, CAS No. 2096984-25-5, also known as Cationic Lipid A), Lipid 8 (Cayman Chemical Item No. 38746, CAS No. 2226547-25-5), RCB-4-8 (Cayman Chemical Item No. 38803, CAS No. 2941228-91-5), YK-009 (Cayman Chemical Item No. 38282, CAS No. 2761458-86-8), 4A3-SC8 (Cayman Chemical Item No. 38155, CAS No. 1857340-78-3), Lipid 2,2(8,8) 4C CH3 (Cayman Chemical Item No. 37910, CAS No. 2230647-30-8, also known as ATX-0114), ATX-001 (Cayman Chemical Item No. 39037, CAS No. 1777792-33-2), ALC-0315 analogous-1 (Cayman Chemical Item No. 38591, CAS No. 2430034-17-4), C14-SPM (Cayman Chemical Item No. 38785, CAS No. 2241864-59-3, also known as C14-spermine), Lipid 222 (Cayman Chemical Item No. 38338), Lipid A4 (Cayman Chemical Item No. 38351, CAS No. 2639634-71-0), C14-4 (Cayman Chemical Item No. 38942, CAS No. 2639634-80-1), A12-Iso5-2DC18 (Cayman Chemical Item No. 38586, CAS No. 2412492-06-7), 1,2-Dipalmitoyl-3-dimethylammonium-propane (Cayman Chemical Item No. 38311, CAS No. 96326-74-8, also known as 16:0 DAP or DPDAP), 1,2-Dimyristoyl-3-dimethylammonium-propane (Cayman Chemical Item No. 38310, CAS No. 72719-84-7, also known as 14:0 DAP or DMDAP), Lipid CL1 (Cayman Chemical Item No. 38320, CAS No. 1450888-71-7), L202 (Cayman Chemical Item No. 37841, CAS No. 2170488-92-1), C13-112-tetra-tail (Cayman Chemical Item No. 38329, CAS No. 1381861-92-2), Lipid 14 (Cayman Chemical Item No. 38589, CAS No. 2430034-05-0), OF-Deg-Lin (Cayman Chemical Item No. 37853, CAS No. 1853202-95-5), DOG-IM4 (Cayman Chemical Item No. 37441, CAS No. 2758097-38-8), AL-A12 (Cayman Chemical Item No. 38001), 98N12-5 (Cayman Chemical Item No. 37651, CAS No. 917572-74-8, also known as ND98), TT3 (Cayman Chemical Item No. 37909, CAS No. 1821214-50-9), 246C10 (Cayman Chemical Item No. 37907, CAS No. 2635329-26-7), OF-C4-Deg-Lin (Cayman Chemical Item No. 37856, CAS No. 1853203-01-6), Lipid A6 (Cayman Chemical Item No. 35052), IC8 (Cayman Chemical Item No. 37986, CAS No. 2349307-32-8), PPZ-A10 (Cayman Chemical Item No. 9004144), AA3-DLin (Cayman Chemical Item No. 37903), BAMEA-O16B (Cayman Chemical Item No. 37439, CAS No. 2490668-30-7, also known as BAMPA-O16B), 113-016B (Cayman Chemical Item No. 37831, CAS No. 2566523-07-5), ssPalmO-Phe (Cayman Chemical Item No. 37670, CAS No. 2377474-67-2), ssPalmM (Cayman Chemical Item No. 37377, CAS No. 1436860-60-4), Lipid A9 (Cayman Chemical Item No. 37667, CAS No. 2036272-50-9), OF-02 (Cayman Chemical Item No. 37652, CAS No. 1883431-67-1), 113-O12B (Cayman Chemical Item No. 37671, CAS No. 2803699-72-9), ATX-100 (Cayman Chemical Item No. 36935, CAS No. 2230647-37-5), CL4H6 (Cayman Chemical Item No. 37279, CAS No. 2256087-35-9), 9A1P9 (Cayman Chemical Item No. 37276, CAS No. 2760467-57-8), 80-O16B (Cayman Chemical Item No. 37564, CAS No. 1624618-02-5), CIN-16645 (Cayman Chemical Item No. 37278, CAS No. 1799316-64-5, also known as LP-01), 306-O12B (Cayman Chemical Item No. 37549, CAS No. 2566523-06-4), 306-O12B-3 (Cayman Chemical Item No. 37096), Lipid C24 (Cayman Chemical Item No. 37122, CAS No. 2767561-52-2), cKK-E12 (Cayman Chemical Item No. 36700, CAS No. 1432494-65-9), NT1-O14B (Cayman Chemical Item No. 37095, CAS No. 2739805-64-0), TCL053 (Cayman Chemical Item No. 37045, CAS No. 2361162-70-9), C12-200 (Cayman Chemical Item No. 36699, CAS No. 1220890-25-4), DLin-DMA (Cayman Chemical Item No. 36701, CAS No. 871258-12-7, also known as 1,2-Dilinoleyloxy-N,N-dimethyl-3-aminopropane), YSK05 (Cayman Chemical Item No. 35786, CAS No. 1318793-78-0), 306Oᵢ₁₀ (Cayman Chemical Item No. 36698, CAS No. 2322290-93-5), Lipid 29 (Cayman Chemical Item No. 35337, CAS No. 2244716-55-8), L-319 (Cayman Chemical Item No. 35051, CAS No. 1351586-50-9), 93-O17O (Cayman Chemical Item No. 34366, CAS No. 2227214-78-8), ALC-0315 (Cayman Chemical Item No. 34337, CAS No. 2036272-55-4), 93-O17S (Cayman Chemical Item No. 34367, CAS No. 2227008-67-3), Lipid 5 (Cayman Chemical Item No. 34372, CAS No. 2089251-33-0), DLin-MC3-DMA (Cayman Chemical Item No. 34364, CAS No. 1224606-06-7, also known as MC3), 1,2-Dioleyloxy-3-dimethylamino-propane (Cayman Chemical Item No. 15109, CAS No. 104162-47-2, also known as DODMA or MBN 305A), 1,2-Dioleoyl-3-dimethylammonium-propane (Cayman Chemical Item No. 25726, CAS No. 127512-29-2, also known as 18:1 DAP or DODAP), SM-102 (Cayman Chemical Item No. 33474, CAS No. 2089251-47-6, also known as Lipid H or LNP-102), DLin-KC2-DMA (Cayman Chemical Item No. 34363, CAS No. 1190197-97-7, also known as KC2), Lipid 8 (see FIG. 31) a variant or derivative thereof, or a combination thereof.

In some aspects, the LNP of the present disclosure comprises Lipid 8 (see FIG. 31), Lipid 10 (see FIG. 31), or a combination thereof. In some aspects, the LNP of the present disclosure does not contain MC3. In some aspects, the LNP of the present disclosure does not contain KC2. In some aspects, the LNP of the present disclosure does not contain cKK-E12.

In some aspects, the LNP of the present disclosure comprises Lipid 10 (see FIG. 31). Lipid 10 containing LNPs can deliver their payload efficiently to T cells, NK cells and monocytes, but not to liver cells or to B cells. Accordingly, in some aspects the present disclosure provides LNP comprising Lipid 10 for delivery of a payload to a T cell. In some aspects, the present disclosure provides LNP comprising Lipid 10 for delivery of a payload to a NK cell. In some aspects, the present disclosure provides LNP comprising Lipid 10 for delivery of a payload to a monocyte. In some aspects, the present disclosure provides LNPs comprising Lipid 10, wherein the LNPs' payload is not delivered to liver cells or to B cells.

### I.A.ii Structural Lipids

As used herein, the term “structural lipid” refers to sterols and to lipids containing sterol moieties. Incorporation of structural lipids in the LNP may help mitigate aggregation of other lipids in the particle.

In some aspects, the structural lipid is selected from the group consisting of cholesterol, beta-cholesterol, ergosterol, 7-dehydrocholesterol, 24S-hydroxycholesterol, lanosterol, cycloartenol, fucosterol, saringosterol, campesterol, 0-sitosterol, sitostanol, coprostanol, avenasterol, stigmasterol, and any combination thereof. Other sterols suitable for use as structural lipids comprise cholesterol sulfate, desmosterol-d6, lathosterol-d7, desmosterol, dihydrolanosterol, zymosterol, lathosterol, zymosterol-d5,14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, lanosterol-95, dihydro FF-MAS-d6, zymostenol-d7, zymostenol, campestanol, 7-dehydrodesmosterol, pregnenolone, sitosterol-d7, dihydro T-MAS, delta 5-avenasterol, brassicasterol, dihydro FF-MAS, 24-methylene cholesterol, cholic acid derivatives, cholesteryl esters, glycosylated sterols, hopanoids, hydroxysteroid, phytosterol, zoosterol, gonane, dexamethasone, and medrogestone. In particular aspects, the LNP of the present disclosure comprise cholesterol.

### I.A.iii Helper Lipids

The term “helper lipid”, as used herein, refers to lipids other than the cationic or ionizable cationic lipids and stabilizing lipid (generally PEG-conjugated lipids) that can influence the properties of the LNP. Helper lipids function to stabilize and improve processing of LNPs.

In some aspects, the helper lipid is a phospholipid. A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. Phospholipids can be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospholipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain).

In some aspects, the helper lipid comprises at least one symmetric phospholipid. In some aspects, the symmetric phospholipid comprises or consists of a symmetric phosphocholine. In some aspects, the symmetric phosphocholine is selected from the group consisting of 1,2-dipropionyl-sn-glycero-3-phosphocholine (03:0 PC); 1,2-dibutyryl-sn-glycero-3-phosphocholine (04:0 PC); 1,2-dipentanoyl-sn-glycero-3-phosphocholine (05:0 PC); 1,2-dihexanoyl-sn-glycero-3-phosphocholine (06:0 PC); 1,2-diheptanoyl-sn-glycero-3-phosphocholine (07:0 PC); 1,2-dioctanoyl-sn-glycero-3-phosphocholine (08:0 PC); 1,2-dinonanoyl-sn-glycero-3-phosphocholine (09:0 PC); 1,2-didecanoyl-sn-glycero-3-phosphocholine (10:0 PC); 1,2-diundecanoyl-sn-glycero-3-phosphocholine (11:0 PC, DUPC); 1,2-dilauroyl-sn-glycero-3-phosphocholine (DLPC); 1,2-dilinoleoyl-sn-glycero-3-phosphocholine (DLOPC); 1,2-ditridecanoyl-sn-glycero-3-phosphocholine (13:0 PC); 1,2-dimyristoyl-sn-glycero-3-phosphocholine (14:0 PC, DMPC); 1,2-dipentadecanoyl-sn-glycero-3-phosphocholine (15:0 PC); 1,2-dipalmitoyl-sn-glycero-3-phosphocholine (16:0 PC, DPPC); 1,2-diphytanoyl-sn-glycero-3-phosphocholine (4ME 16:0 PC); 1,2-diheptadecanoyl-sn-glycero-3-phosphocholine (17:0 PC); 1,2-distearoyl-sn-glycero-3-phosphocholine (18:0 PC, DSPC); 1,2-dinonadecanoyl-sn-glycero-3-phosphocholine (19:0 PC); 1,2-diarachidoyl-sn-glycero-3-phosphocholine (20:0 PC); 1,2-dihenarachidoyl-sn-glycero-3-phosphocholine (21:0 PC); 1,2-dibehenoyl-sn-glycero-3-phosphocholine (22:0 PC); 1,2-ditricosanoyl-sn-glycero-3-phosphocholine (23:0 PC); 1,2-dilignoceroyl-sn-glycero-3-phosphocholine (24:0 PC); 1,2-dimyristoleoyl-sn-glycero-3-phosphocholine (14:1 (A9-Cis) PC); 1,2-dimyristelaidoyl-sn-glycero-3-phosphocholine (14:1 (A9-Trans) PC); 1,2-dipalmitoleoyl-sn-glycero-3-phosphocholine (16:1 (A9-Cis) PC); 1,2-dipalmitelaidoyl-sn-glycero-3-phosphocholine (16:1 (A9-Trans) PC); 1,2-dipetroselenoyl-sn-glycero-3-phosphocholine (18:1 (A6-Cis) PC); 1,2-dioleoyl-sn-glycero-3-phosphocholine (18:1 (A9-Cis) PC, DOPC); 1,2-dielaidoyl-sn-glycero-3-phosphocholine (18:1 (A9-Trans) PC); 1,2-dilinoleoyl-sn-glycero-3-phosphocholine (18:2 (Cis) PC, DLPC); 1,2-dilinolenoyl-sn-glycero-3-phosphocholine (18:3 (Cis) PC, DLnPC); 1,2-dieicosenoyl-sn-glycero-3-phosphocholine (20:1 (Cis) PC); 1,2-diarachidonoyl-sn-glycero-3-phosphocholine (20:4 (Cis) PC, DAPC); 1,2-dierucoyl-sn-glycero-3-phosphocholine (22:1 (Cis) PC); 1,2-didocosahexaenoyl-sn-glycero-3-phosphocholine (22:6 (Cis) PC, DHAPC); 1,2-dinervonoyl-sn-glycero-3-phosphocholine (24:1 (Cis) PC); 1,2-dierucoyl-sn-glycero-3-phosphocholine (DEPC); 1,2-di-O-octadecenyl-sn-glycero-3-phosphocholine (18:0 diether PC); and any combination thereof.

In some aspects, the symmetric phosphocholine is DSPC and/or DOPC. In some aspects, the symmetric phospholipid comprises or consists of a symmetric phosphoethanolamine (PE). In some aspects, the symmetric phosphoethanolamine is selected from the group consisting of: 1,2-dihexanoyl-sn-glycero-3-phosphoethanolamine (06:0 PE); 1,2-dioctanoyl-sn-glycero-3-phosphoethanolamine (08:0 PE); 1,2-didecanoyl-sn-glycero-3-phosphoethanolamine (10:0 PE); 1,2-dilauroyl-sn-glycero-3-phosphoethanolamine (12:0 PE); 1,2-dimyristoyl-sn-glycero-3-phosphoethanolamine (14:0 PE, DMPE); 1,2-dipentadecanoyl-sn-glycero-3-phosphoethanolamine (15:0 PE); 1,2-dipalmitoyl-sn-glycero-3-phosphoethanolamine (16:0 PE, DPPE); 1,2-diphytanoyl-sn-glycero-3-phosphoethanolamine (4ME 16:0 PE); 1,2-diheptadecanoyl-sn-glycero-3-phosphoethanolamine (17:0 PE); 1,2-distearoyl-sn-glycero-3-phosphoethanolamine (18:0 PE, DSPE); 1,2-dipalmitoleoyl-sn-glycero-3-phosphoethanolamine (16:1 PE); 1,2-dioleoyl-sn-glycero-3-phosphoethanolamine (18:1 (A9-Cis) PE, DOPE); 1,2-dielaidoyl-sn-glycero-3-phosphoethanolamine (18:1 (A9-Trans) PE); 1,2-dilinoleoyl-sn-glycero-3-phosphoethanolamine (18:2 PE, DLPE); 1,2-dilinolenoyl-sn-glycero-3-phosphoethanolamine (18:3 PE, DLnPE); 1,2-diarachidonoyl-sn-glycero-3-phosphoethanolamine (20:4 PE, DAPE); 1,2-didocosahexaenoyl-sn-glycero-3-phosphoethanolamine (22:6 PE, DHAPE); 1,2-dierucoyl-sn-glycero-3-phosphoethanolamine (DEPE); and. any combination thereof.

In some aspects, the symmetric phosphoethanolamine is DSPE and/or DOPE. In some aspects, the helper lipid can comprise an asymmetric phospholipid such as MPPC, MSPC, PMPC, PSPC, POPC, PLPC, SMPC, SPPC, SOPC, OMPC, OPPC, OSPC, POPE, or a combination thereof. In some aspects, the helper lipid can comprise a lysolipid, e.g., a lyso PC or a lyso PE. Lysophospholipids are derivatives of a phospholipid in which one or both fatty acyl chains have been removed by hydrolysis. In some aspects, the helper lipid can comprise a phosphoglycerol (PG), such as DEPG, DLPG, DMPG, DOPG or DPPG; a phosphoserine (PS), such as DEPS, DLPS, DMPS, DOPS or DPPS; a phosphatidic acid (PA), such as DEPA, DLPA, DMPA, DOPA, or DPPA, or a combination thereof.

### I.A.iv PEG-Lipids

As used herein, the term “PEG-modified lipid” or “PEG-lipid” refers to a lipid-linked (e.g., covalently attached) to at least one PEG polymer chain. In some aspects, the PEG-lipid described herein comprises a poly(ethylene)glycol chain of up to 5, 10 or 20 kDa in length covalently attached to a lipid with alkyl chain(s) of C6-C20 length. In some aspects, the PEG-lipid is reversibly linked to the LNP described herein and the PEG moiety is gradually released in blood circulation upon administration. In some aspects, an alternative to a PEG-lipid can be used, for example, a derivatized lipid such as a derivatized ceramide (PEG-CER), including N-Octanoyl-Sphingosine-1-[Succinyl(Methoxy Polyethylene Glycol)-2000](C8 PEG-2000 ceramide). In some aspects, the PEG-lipid described herein comprises or consists of a PEG-phospholipid and/a PEG-ceramide. In some aspects, the PEG-lipid is a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or a combination thereof. In some aspects, the PEG-lipid is selected from the group consisting of PEG-c-DOMG, PEG-DMG, PEG-DLPE, PEG-DMPE, PEG-DPPC, PEG-DSPE, and any combination thereof. In some aspects, the PEG-lipid is DMG-PEG2000. In some aspects, the PEG-lipid is DSPE-PEG2000. In some aspects, the PEG-lipid is a ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide. In some aspects, the PEG-lipid is a PEG derivative, such as 16:0 PEG5000 PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE, 16:0 PEG1000 PE, 14:0 PEG1000 PE, 18:0 PEG1000 PE, 18:1 PEG1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350. In some aspects, the PEG-lipid is a sterol PEG derivative such as Chol-PEG600. In some aspects, the PEG-lipid is a glycerol PEG derivative such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some aspects, the PEG-lipid is a phospholipid PEG derivative such as DSPE-PEG5000, DSPE-PEG2000, DSPE-PEG1000, or DSPE-PEG550.

PEG-modified lipids include, but are not limited to, a polyethylene glycol chain of up to 5 kDa in length covalently attached to a lipid with alkyl chain(s) of C6-C20 length. In some aspects, the PEG-lipid employed in the compositions and methods of the present disclosure is 1,2-dimyristoyl-sn-glycerol, methoxypolyethylene Glycol (2000 MW PEG) “DMG-PEG2000.”

The addition of PEG-modified lipids to the lipid delivery vehicle may prevent complex aggregation and may also provide a means for increasing circulation lifetime and increasing the delivery of the lipid-polynucleotide composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). In some aspects, the lipid moiety of the PEG-lipids includes those having lengths of from about C₁₄ to about C₂₂, such as from about C₁₄ to about C₁₆. In some aspects, a PEG moiety, for example an mPEG-NH2, has a size of about 1000, 2000, 5000, 10,000, 15,000 or 20,000 daltons. In some aspects, the PEG-lipid is a non-diffusible PEG conjugates. Non-limiting examples of non-diffusible PEG conjugates include PEG-DSG and PEG-DSPE.

### I.A.v Chemically Modified Lipids

In some aspects, the LNP comprises a chemically modified lipid. As used herein, the term “chemically modified lipid” refers to a lipid that has been modified to be derivatizable by incorporating a chemically reactive group (e.g., a maleimide group or a sulfhydryl group) that can be used to attach a biologically active moiety (e.g., an antibody) covalently (e.g., via reaction between the maleimide group and a sulfhydryl group) or non-covalently to the lipid.

In some aspects, the LNP further comprises a derivatizable lipid, e.g., a PEG lipid comprising a maleimide group such as DSPE-PEG2000-maleimide) wherein the maleimide reactive group is free (i.e., prior to the reaction with an antibody). In some aspects, the derivatizable lipid is conjugated to a T cell targeting molecule (e.g., an antibody, such as a bispecific anti CD3/anti CD28 antibody) thereby anchoring the T cell targeting molecule to the surface of the LNP.

In some aspects, where a certain class of lipids present in a LNP of the present disclosure includes both a chemically modified lipid and an unmodified lipid, the chemically modified lipid can be derived from the unmodified lipid. By way of example, the one or more lipids of a LNP may include an unmodified DSPE-PEG2000 lipid and a modified DSPE-PEG2000 lipid that includes a functionalized group capable of forming a covalent bond, e.g., DMG-PEG2000-maleimide or DSPE-PEG2000-maleimide.

In some aspects, LNP of the present disclosure can comprise DMG-PEG2000-maleimide or DSPE-PEG2000-maleimide. In some aspects, LNP of the present disclosure can comprise DMG-PEG2000-SH or DSPE-PEG2000-SH.

In some aspects, the chemically modified lipid is selected from the group consisting of DSPE-PEG2000-maleimide, DSPE-PEG5000-maleimide, DMG-PEG2000-maleimide, DMG-PEG5000-maleimide, cholesterol-PEG2000-maleimide, cholesterol-PEG5000-maleimide, DSPE-PEG2000-SH, DSPE-PEG5000-SH, DMG-PEG2000-SH, DMG-PEG5000-SH, cholesterol-PEG2000-SH, and cholesterol-PEG5000-SH.

### I.A.vi Other Lipid Components

In some aspects, the LNP of the present disclosure can comprise additional components such as fatty acids, lysolipids, or vitamins. In some aspects, the fatty acid is a short-chain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as a ω-3 (omega-3) or ω-6 (omega-6) fatty acid.

### I.A.vii Specific LNP Compositions

In some aspects, the LNP of the present disclosure contains a molar ratio (mol %) of the cationic or ionizable cationic lipid or lipidoid (e.g., cKK-E12, ALC-0315, SSOP, SM-102, MC3, KC2, Lipid 8 (FIG. 31), Lipid 10 (FIG. 31), Trialkyl Lipid 10, any one of MDX1-MDX13 (FIG. 57-59), or a combination thereof) from about 30% to about 60% mol %. In some aspects, the molar ratio of the cationic or ionizable cationic lipid or lipidoid (e.g., cKK-E12, ALC-0315, KC2, MC3, SSOP, SM-102, Lipid 8 (FIG. 31), Lipid 10 (FIG. 31), Trialkyl Lipid 10, any one of MDX1-MDX13 (FIG. 57-59), or a combination thereof) is about 30%, about 35%, about 40%, about 45%, about 50%, about 55%, or about 60%. In some aspects, the molar ratio of the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) is between about 30% and about 35%, between about 35% and 40%, between about 40% and about 45%, between about 45% and about 50%, between about 50% and about 55%, or between about 55% and about 60%. In some aspects, the molar ratio of the cationic or ionizable cationic lipid or lipidoid (e.g., cKK-E12, ALC-0315, SSOP, SM-102, MC3, KC2, Lipid 8, Lipid 10, Trialkyl Lipid 10, any one of MDX1-MDX13, or a combination thereof) is between 30% and about 60%, between about 35% and about 55%, or between about 40% and about 50%. In some aspects, the molar ratio of the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) is about 35%.

In some aspects, the LNP of the present disclosure contains a molar ratio (mol %) of the structural lipid (e.g., cholesterol, beta-cholesterol, or a combination thereof) from about 20% to about 60% mol %. In some aspects, the molar ratio of the structural lipid (e.g., cholesterol) is about 20%, about 25%, about 30%, about 35%, about 40%, about 45%, about 50%, about 55%, or about 60%. In some aspects, the molar ratio of the structural lipid (e.g., cholesterol) is between about 20% and about 25%, about 25% and about 30%, about 30% and about 35%, between about 35% and 40%, between about 40% and about 45%, between about 45% and about 50%, between about 50% and about 55%, or between about 55% and about 60%. In some aspects, the molar ratio of the structural lipid (e.g., cholesterol) is between 20% and about 60%, between about 25% and about 55%, or between about 30% and about 50%, or between about 35% and about 45%. In some aspects, the molar ratio of the structural lipid (e.g., cholesterol) is about 46.5%.

In some aspects, the LNP contains a molar ratio (mol %) of total phospholipid from about 5% to about 30% mol %. As used herein total phospholipid refers to the total amount of helper lipid (e.g., DSPE, DSPC, DOPE, DOPE, or a combination thereof), PEG lipid (e.g., a DMG-PEG2000, DSPE-PEG2000, or a combination thereof), and chemically modified lipid (e.g., DSPE-PEG2000-maleimide). In some aspects, the molar ratio of total phospholipid is about 5%, about 10%, about 15%, about 20%, about 25%, or about 30%. In some aspects, the molar ratio of total phospholipid is between about 5% and about 10%, about 10% and about 15%, about 15% and about 20%, about 20% and about 25%, or about 25% and about 30%. In some aspects, the molar ratio of total phospholipid is between about 5% and about 30%, about 10% and about 25%, and about 15% and about 20%. In some aspects, the molar ratio of total phospholipid is about 18.5% (comprising about 16% of helper lipid, about 2% of PEG-lipid, and about 0.5% of chemically modified lipid).

In some aspects, the LNP of the present disclosure comprises (1) a molar ratio (mol %) of the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) from about 30% to about 60% mol %; (2) a molar ratio (mol %) of the structural lipid (e.g., cholesterol, beta-cholesterol, or a combination thereof) from about 20% to about 60% mol %; and, (3) a molar ratio (mol %) of total phospholipid from about 5% to about 30% mol %.

In some aspects, the LNP of the present disclosure comprises: (1) a molar ratio of the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) of about 30%, about 35%, about 40%, about 45%, about 50%, about 55%, or about 60%; (2) a molar ratio of the structural lipid (e.g., cholesterol) of about 20%, about 25%, about 30%, about 35%, about 40%, about 45%, about 50%, about 55%, or about 60%; and, (3) a molar ratio of total phospholipid about 5%, about 10%, about 15%, about 20%, about 25%, or about 30%.

In some aspects, the LNP of the present disclosure comprises: (1) a molar ratio of the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) between about 30% and about 35%, between about 35% and 40%, between about 40% and about 45%, between about 45% and about 50%, between about 50% and about 55%, or between about 55% and about 60%; (2) a molar ratio of the structural lipid (e.g., cholesterol) between about 20% and about 25%, about 25% and about 30%, about 30% and about 35%, between about 35% and 40%, between about 40% and about 45%, between about 45% and about 50%, between about 50% and about 55%, or between about 55% and about 60%; and, (3) a molar ratio of total phospholipid between about 5% and about 10%, about 10% and about 15%, about 15% and about 20%, about 20% and about 25%, or about 25% and about 30%.

In some aspects, the LNP of the present disclosure comprises: (1) a molar ratio of the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) between 30% and about 60%, between about 35% and about 55%, or between about 40% and about 50%; (2) a molar ratio of the structural lipid (e.g., cholesterol) between 20% and about 60%, between about 25% and about 55%, or between about 30% and about 50%, or between about 35% and about 45%; and, (3) a molar ratio of total phospholipid between about 5% and about 30%, about 10% and about 25%, and about 15% and about 20%.

In some aspects, the LNP of the present disclosure comprises: (1) a molar ratio of the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) of about 35%; (2) a molar ratio of the structural lipid (e.g., cholesterol) of about 46.5%; and, (3) a molar ratio of total phospholipid of about 18.5% (comprising about 16% of helper lipid, about 2% of PEG-lipid, and about 0.5% of chemically modified lipid).

In some aspects, the weight ratio (w/w) between the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) and the payload (e.g., a nucleic acid such as mRNA) is from about 5% to about 15%. In some aspects, the weight ratio (w/w) between the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) and the payload (e.g., a nucleic acid such as mRNA) is about 5%, about 6%, about 7%, about 8%, about 9%, about 10%, about 11%, about 12%, about 13%, about 14%, or about 15%. In some aspects, the weight ratio (w/w) between the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) and the payload (e.g., a nucleic acid such as mRNA) is between about 5% and about 6%, about 6% and about 7%, about 7% and about 8%, about 8% and about 9%, about 9% and about 10%, about 10% and about 11%, about 11% and about 12%, about 12% and about 13%, about 13% and about 14%, or about 14% and about 15%. In some aspects, the weight ratio (w/w) between the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) and the payload (e.g., a nucleic acid such as mRNA or a DNA, e.g., linear DNA) is between about 5% and about 15%, about 6% and about 14%, about 7% and about 13%, about 8% and about 12%, or about 9% and about 11%. In some aspects, the weight ratio (w/w) between the cationic or ionizable cationic lipid or lipidoid (e.g., cKK-E12, ALC-0315, KC2, MC3, SSOP, SM-102, Lipid 8, Lipid 10, Trialkyl Lipid 10, any one of MDX1-MDX13, or a combination thereof) and the payload (e.g., a nucleic acid such as mRNA) about 18.5% (comprising about 16% of helper lipid, about 2% of PEG-lipid, and about 0.5% of chemically modified lipid).

In some aspects, the LNP comprises a molar ratio (mol %) of cholesterol of about 46.5%. In some aspects, the LNP comprises a molar ratio (mol %) of cholesterol of about 40%, about 41%, about 42%, about 43%, about 44%, about 45%, about 46%, about 47%, about 48%, about 49%, about 50%, about 51% or about 52%.

In some aspects, the LNP comprises a molar ratio (mol %) of DSPE-PEG2000-maleimide of about 0.5%. In some aspects, the LNP comprises a molar ratio (mol %) of DSPE-PEG2000-maleimide of about 0.1%, about 0.2%, about 0.3%, about 0.4%, about 0.5%, about 0.6%, about 0.7%, about 0.8%, about 0.9%, or about 1%.

In some aspects, the LNP comprises a molar ratio (mol %) of DOPE of about 16%. In some aspects, the LNP comprises a molar ratio (mol %) of DOPE of about 10%, about 11%, about 12%, about 13%, about 14%, about 15%, about 16%, about 17%, about 18%, about 19%, about 20%, about 21% or about 22%.

In some aspects, the LNP comprises a molar ratio (mol %) of cKK-E12, ALC-0315, KC2, MC3, SSOP, SM-102, Lipid 8, Lipid 10, Trialkyl Lipid 10, any one of MDX1-MDX13, or a combination thereof of about 35%. In some aspects, the LNP comprises a molar ratio (mol %) of cKK-E12, ALC-0315, KC2, MC3, SSOP, SM-102, Lipid 8, Lipid 10, Trialkyl Lipid 10, any one of MDX1-MDX13, or a combination thereof of about 30%, about 31%, about 32%, about 33%, about 34%, about 35%, about 36%, about 37%, about 38%, about 39%, or about 40%.

In some aspects, the LNP comprises a molar ratio (mol %) of DMG-PEG2000 of about 2%. In some aspects, the LNP comprises a molar ratio (mol %) of DMG-PEG2000 of about 1%, about 1.2%, about 1.4%, about 1.6%, about 1.8%, about 2%, about 2.2%, about 2.4%, about 2.6%, about 2.8%, or about 3%.

In some aspects, the LNP comprises: (1) a molar ratio (mol %) of cholesterol of about 46.5%; (2) a molar ratio (mol %) of DSPE-PEG2000-maleimide of about 0.1 to about 0.5%; (3) a molar ratio (mol %) of DOPE of about 16%; (4) a molar ratio (mol %) of cKK-E12, ALC-0315, SSOP, SM-102, MC3, KC2, Lipid 8, Lipid 10, Trialkyl Lipid 10, any one of MDX1-MDX13, or a combination thereof of about 35%; and, (5) a molar ratio (mol %) of DMG-PEG2000 of about 2.0 to about 2.4%.

In some aspects, the LNP comprises: (1) a molar ratio (mol %) of cholesterol of about 51.9%; (2) a molar ratio (mol %) of DSPE-PEG2000-maleimide of about 0.6%; (3) a molar ratio (mol %) of DOPE of about 8.8%; (4) a molar ratio (mol %) of cKK-E12, ALC-0315, SSOP, SM-102, MC3, KC2, Lipid 8, Lipid 10, Trialkyl Lipid 10, any one of MDX1-MDX13, or a combination thereof of about 36.5%; and, (5) a molar ratio (mol %) of DMG-PEG2000 of about 2.2%.

In some aspects, the LNP comprises: (1) a molar ratio (mol %) of cholesterol of about 38.5%; (2) a molar ratio (mol %) of DSPE-PEG2000-maleimide of about 0.2%; (3) a molar ratio (mol %) of DOPE, DSPC, or a combination thereof of about 10%; (4) a molar ratio (mol %) of cKK-E12, ALC-0315, KC2, MC3, SSOP, SM-102, Lipid 8, Lipid 10, Trialkyl Lipid 10, any one of MDX1-MDX13, or a combination thereof of about 50%; and, (5) a molar ratio (mol %) of DMG-PEG2000 of about 1.3%.

In some aspects, the LNP comprises: (1) a molar ratio (mol %) of cholesterol of about 40%, about 41%, about 42%, about 43%, about 44%, about 45%, about 46%, about 47%, about 48%, about 49%, about 50%, about 51% or about 52%; (2) a molar ratio (mol %) of DSPE-PEG2000-maleimide of about 0.1%, about 0.2%, about 0.3%, about 0.4%, about 0.5%, about 0.6%, about 0.7%, about 0.8%, about 0.9%, or about 1%; (3) a molar ratio (mol %) of DOPE of about 10%, about 11%, about 12%, about 13%, about 14%, about 15%, about 16%, about 17%, about 18%, about 19%, about 20%, about 21% or about 22%; (4) a molar ratio (mol %) of cKK-E12, AL-0315, KC2, MC3, SSOP, SM-102, Lipid 8, Lipid 10, Trialkyl Lipid 10, any one of MDX1-MDX13, or a combination thereof of about 30%, about 31%, about 32%, about 33%, about 34%, about 35%, about 36%, about 37%, about 38%, about 39%, or about 40%; and, (5) a molar ratio (mol %) of DMG-PEG2000 of about 1%, about 1.2%, about 1.4%, about 1.6%, about 1.8%, about 2%, about 2.2%, about 2.4%, about 2.6%, about 2.8%, or about 3%.

In some aspects, the LNP comprises: (1) a molar ratio (mol %) of cholesterol of about 46.5±4%; (2) a molar ratio (mol %) of DSPE-PEG2000-maleimide of about 0.5±0.1%; (3) a molar ratio (mol %) of DOPE of about 16%±2%; (4) a molar ratio (mol %) of cKK-E12 (or, e.g., AL-0315, KC2, MC3, SSOP, SM-102, Lipid 8, Lipid 10, Trialkyl Lipid 10, any one of MDX1-MDX13, or a combination thereof) of about 35±4%; and, (5) a molar ratio (mol %) of DMG-PEG2000 of about 2±0.2%.

In some aspects, the LNP comprises (i) a molar ratio of about 50% ionizable cationic lipid, e.g., Lipid 10 or any one of MDX1-MDX13, (ii) a molar ratio of about 10% DSPC, (iii) a molar ratio of about 38.5% cholesterol, and (iv) a molar ratio of about 1.5% DMG-PEG.

In some aspects, the LNP comprises (i) a molar ratio of about 50% Lipid 10, (ii) a molar ratio of about 10% DSPC, (iii) a molar ratio of about 38.5% cholesterol, and (iv) a molar ratio of about 1.5% DMG-PEG.

The selection of cationic or ionizable cationic lipids, structural lipids, PEG-lipids, helper lipids, and chemically modified lipids and lipid conjugates which comprise the LNP, as well as the relative molar ratio of such lipids to each other, is based upon the characteristics of the selected lipid(s), the nature of the intended target cells, and the characteristics of the payload (e.g., nucleic acid) to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

In some aspects, the LNP of the present disclosure comprises GenVoyILM™. In some aspects, the LNP of the present disclosure does not comprise GenVoyILM™. In some aspects, the LNP of the present disclosure does not comprise 50% ionizable cationic lipid, 10% DSPC, 37% cholesterol, and 2.5% stabilizing lipid. In some aspects, the LNP of the present disclosure does not comprise 50% ionizable cationic lipid. In some aspects, the LNP of the present disclosure does not comprise 10% DSPC. In some aspects, the LNP of the present disclosure does not comprise 37%% cholesterol. In some aspects, the LNP of the present disclosure does not comprise 2.5% stabilizing lipid. See Kitte et al. Molecular Therapy: Methods & Clinical Development (2023), doi.org/10.1016/j.omtm.2023.101139, which is herein incorporated by reference in its entirety.

In some aspects, the LNP of the present disclosure does not have an average Mw in g/mol of approximately 630.5 Da. In some aspects, the LNP of the present disclosure does not have Z-Avg diameter of 45-75 nm when the payload is siRNA. In some aspects, the LNP of the present disclosure does not have a Z-Avg diameter pf 60-120 nm when the payload is mRNA. In some aspects, the target LNP delivery systems of the present disclosure is not a LNP composition disclosed in WO2023057979A1, WO2018119514A1, WO2020210901A1, WO2020206231A1, WO2018064755A1, WO2020252589A1, WO2021000041A1, or WO2019210394A1, which are herein incorporated by reference in their entireties.

The LNP for use in the method of the invention can be prepared by various techniques that are presently known in the art. Nucleic acid-lipid particles and their method of preparation are disclosed in, for example, U.S. Patent Publication Nos. 2004/0142025 and 2007/0042031, the disclosures of which are herein incorporated by reference in their entirety for all purposes. Selection of the appropriate size of LNP must take into consideration the site of the target cell and the application for which the LNP is being made.

Generally, the LNP have a size (e.g., average hydrodynamic diameter) within the range of about 25 to about 500 nm. In some aspects, the LNP has a size (e.g., average hydrodynamic diameter) from about 50 nm to about 300 nm, or from about 60 nm to about 120 nm. In some aspects, the average size of the LNP (e.g., average hydrodynamic diameter) is between about 85 nm and about 110 nm. In some aspects, the LNP have a size (e.g., average hydrodynamic diameter) of about 80 nm, about 85 nm, about 90 nm, about 95 nm, about 100 nm, about 105 nm, about 110 nm, about 115 nm, or about 120 nm. In some aspects, the average size of the LNP (e.g., average hydrodynamic diameter) is 95±5 nm. In some aspects, the LNP has a size (nm) of about 100 nm, about 110 nm, about 120 nm, about 130 nm, about 140 nm or about 150 nm. In some aspects, the LNP has a size (nm) of about 90 nm to about 100 nm, about 100 nm to about 110 nm, about 110 nm to about 120 nm, about 120 nm to about 130 nm, about 130 to about 140 nm, about 140 nm to about 150 nm, about 90 nm to about 110 nm, about 100 nm to about 120 nm, about 110 nm to about 130 nm, about 120 nm to about 140 nm, about 130 to about 150 nm, about 90 to about 120 nm, about 100 to about 130 nm, about 110 nm to about 140 nm, about 120 nm to about 150 nm, about 90 nm to about 130 nm, about 100 nm to about 140 nm, about 110 to about 150 nm, about 90 to about 140 nm, about 100 to about 150 nm, or about 90 nm to about 150 nm.

A LNP composition may be relatively homogenous. A polydispersity index may be used to indicate the homogeneity of a LNP composition, e.g., the particle size distribution of the LNP compositions. A small (e.g., less than 0.3) polydispersity index generally indicates a narrow particle size distribution. In some aspects, the polydispersity index of the LNP of the present disclosure is less than about 0.3, less than about 0.29, less than about 0.28, less than about 0.27, less than about 0.26, less than about 0.25, less than about 0.24, less than about 0.23, less than about 0.22, less than about 0.21, less than about 0.2, less than about 0.19, less than about 0.18, less than about 0.17, less than about 0.16, or less than about 0.15. In some aspects, the polydispersity index of the LNP is about 0.29, about 0.28, about 0.27, about 0.26, about 0.25, about 0.24, about 0.23, about 0.22, about 0.21, about 0.2, about 0.19, about 0.18, about 0.17, about 0.16, or about 0.15.

The zeta potential of a LNP composition may be used to indicate the electrokinetic potential of the composition. For example, the zeta potential may describe the surface charge of a LNP composition. LNP compositions with relatively low charges, positive or negative, are generally desirable, as more highly charged species may interact undesirably with cells, tissues, and other elements in the body. In some aspects, the zeta potential of the LNP of the present disclosure is from about −40 mV to about +40 mV. In some aspects, the zeta potential of the LNP of the present disclosure is from about −10 mV to about +10 mV. In some aspects, zeta potential of the LNP is about −40 mV, about −35 mV, about −30 mV, about −25 mV, about −20 mV, about −15 mV, about −10 mV, about −5 mV, about 0 mV, about +5 mV, about +10 mV, about +15 mV, about +20 mV, about +25 mV, about +30 mV, about +35 mV or about +40 mV. In some aspects, zeta potential of the LNP is between about −40 mV and about −35 mV, between about −35 mV and about −30 mV, between about −30 mV and about −25 mV, between about −25 mV and about −20 mV, between about −20 mV and about −15 mV, between about −15 mV and about −10 mV, between about −10 mV and about −5 mV, between about −5 mV and about 0 mV, between about 0 mV and about +5 mV, between about +5 mV and about +10 mV, between about +10 mV and about +15 mV, between about +15 mV and about +20 mV, between about +20 mV and about +25 mV, between about +25 mV and about +30 mV, between about +30 mV and about +35 mV, or between about +35 mV and about +40 mV.

### I.B Payloads

As used herein, the term “payload” refers to a biologically active molecule (e.g., a therapeutic agent) that acts on a target (e.g., a target cell) that is contacted with a T cell targeted delivery system of the present disclosure. Non-limiting examples of payloads that can be introduced into a T cell targeted delivery system of the present disclosure, include therapeutic agents such as, nucleotides (e.g., therapeutic nucleotides or nucleotides comprising a detectable moiety), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety), polypeptides (e.g., enzymes), lipids, carbohydrates, and small molecules (e.g., small molecule drugs and toxins). In some aspects, a payload comprises an antigen or a nucleic acid (e.g., an mRNA) encoding an antigen. As used herein, the term “antigen” refers to any agent that when introduced into a subject elicits an immune response (cellular or humoral) to itself. In some aspects, the antigen is used to elicit an immune response, i.e., as a vaccine, e.g., in a cancer vaccine. In some aspects, a payload comprises an adjuvant. In some aspects, the payload molecules are covalently linked to the T cell targeted delivery system, e.g., a LNP, via a maleimide moiety.

The T cell targeted delivery systems of the present disclosure can be used to deliver a variety of payload, e.g., therapeutic agents, detectable labels, and cell penetrating payloads. In some aspects, the payloads are encapsulated in the LNP(s). In some aspects, the payload can be covalently or non-covalently linked to the external surface or interior of the LNP, e.g., to the internal membrane or internal surface of the LNP. In some aspects, a payload can be attached to a LNP of the present disclosure via a linker, for example, cleavable linker.

In some aspects of the present disclosure, the payload comprises a polypeptide, a peptide, a polynucleotide, a chemical compound, or any combination thereof. In some aspects, a T cell targeted delivery system of the present disclosure can comprises a single payload. In some aspects, a T cell targeted delivery systems of the present disclosure can comprises multiple payloads.

In some aspects, the payload is a detectable substance. Detectable substances include, but are not limited to, various organic small molecules, inorganic compounds, nanoparticles, enzymes or enzyme substrates, fluorescent materials, luminescent materials, bioluminescent materials, chemiluminescent materials, radioactive materials, and contrast agents. Labels are contemplated by the present disclosure, including, but not limited to, optically detectable labels. Labels can be attached to another payload of the present disclosure, e.g., an mRNA, and/or to a component of the LNP using standard chemistries such that the label can be removed upon cleavage of a cleavable linker. A detectable label can useful in therapeutic, diagnostic, imaging (e.g., radioimaging), or basic research applications.

In some aspects, the detectable label is a radioactive label. Examples of a radioactive label include, but are not limited to, the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc, ¹⁸⁶Re and ⁸⁹Zr.

In some aspects, the detectable label is a chemiluminescent label, fluorescent label, enzyme, biotin, or a combination thereof. In some aspects, the detectable label is a peptide tag. In some aspects, the detectable label is a polyhistidine tag, polyarginine tag, glutathione-S-transferase (GST), maltose binding protein (MBP), chitin binding protein (CBP), Strep-tag, thioredoxin (TRX), poly(NANP), FLAG tag, ALFA-tag, V5-tag, Myc-tag, hemagglutinin (HA) tag, Spot tag, T7 tag, NE tag, or green fluorescence protein (GFP), or a combination thereof. In some aspects, the polyhistidine tag consists of from about 4 to about 10 histidine residues. In some aspects, the polyhistidine tag consists of about 4, about 5, about 6, about 7, about 8, about 9, or about 10 histidine residues. Additional examples of detectable labels and methods for introducing detectable labels into a polypeptide or polynucleotide are known and include routine chemical, molecular biology and recombinant DNA techniques. See, e.g., Hnatowich et al., Science, 220(4597):613-615, 1983; Yao et al., Int. J. Mol. Sci., 17(2):194, 2016; Kimple et al., Curr. Protoc. Protein Sci., 73:Unit 9.9, 2013; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

In some aspects, the payload comprises a therapeutic small molecule. In some aspects, the small molecule is a proteolysis-targeting chimera (PROTAC). In some aspects, the small molecule is a nucleotide, e.g., a stimulator of interferon genes protein (STING) agonist.

In some aspects, the payload comprises, consists or consists essentially of a polynucleotide, e.g., an mRNA, an antisense oligonucleotide (ASO), a phosphorodiamidate morpholino oligonucleotide (PMO), a siRNA, a miRNA, a shRNA, a plasmid, or a vector.

### I.B.i Payloads: Gene Editing System Components

In some aspects, the payload comprises a polynucleotide comprising one or more components of a gene editing system. In some aspects, the payload comprises a gRNA. In some aspects, the payload comprises an mRNA encoding a nuclease. In some aspects, the payload comprises a gRNA and an mRNA encoding a nuclease.

CRISPR/Cas: In some aspects, the gene editing system used with the T cell targeted delivery system can comprise a CRISPR system. In some aspects, the payload of a LNP targeting delivery system of the present disclosure comprises an mRNA encoding a CRISPR Cas nuclease, e.g., an mRNA encoding a Cas9 nuclease.

In some aspects, the CRISPR/Cas nuclease is codon-optimized for the desired cell type in which it is to be expressed. In some aspects, the CRIPS/Cas gene editing system can also employ a guide RNA (gRNA) that comprises two separate molecules. An exemplary two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule.

A crRNA comprises both the DNA-targeting segment (single stranded) of the gRNA and a stretch of nucleotides that forms one-half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence, and a tracrRNA. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used.

In some aspects, the CRISPR/Cas gene editing system can employ a fused crRNA-tracrRNA construct (i.e., a single transcript) that functions with the codon-optimized Cas9. This single RNA is often referred to as a guide RNA or gRNA. Within a gRNA, the crRNA portion is identified as the “target sequence” for the given recognition site and the tracrRNA is often referred to as the “scaffold.” To generate a gRNA, a short DNA fragment containing the target sequence is inserted into a guide RNA expression nucleic acid. The gRNA expression nucleic acid comprises the target sequence (in some aspects around 20 nucleotides), a form of the tracrRNA sequence (the scaffold) as well as a suitable promoter that is active in the cell and necessary elements for proper processing in eukaryotic cells. In some aspects, the payload of the T cell targeted delivery system comprises the gRNA expression nucleic acid comprising the target sequence (in some aspects around 20 nucleotides), a form of the tracrRNA sequence (the scaffold) as well as a suitable promoter. In some aspects, custom, complementary oligonucleotides are annealed to form a double stranded DNA and are then cloned into the gRNA expression nucleic acid, which is included as payload in the T cell targeted delivery system. In some aspects, the payload comprises a two-molecule gRNA or a fused crRNA-tracrRNA construct.

In some aspects, the payload comprises a Cas9 nuclease provided in the form of a protein. In some aspects, the Cas9 protein can be provided in the form of a complex with the gRNA. In some aspects, the Cas9 nuclease can be provided in the form of a nucleic acid encoding the protein. The nucleic acid encoding the Cas9 nuclease can be RNA (e.g., messenger RNA (mRNA)) or DNA. In some aspects, the gRNA can be provided in the form of RNA. In some aspects, the gRNA can be provided in the form of DNA encoding the RNA. In some aspects, the gRNA can be provided in the form of separate crRNA and tracrRNA molecules, or separate DNA molecules encoding the crRNA and tracrRNA, respectively.

In some aspects, the gRNA comprises a third nucleic acid sequence encoding a Clustered Regularly Interspaced Short Palindromic Repeats (CRISPR) RNA (crRNA) and a trans-activating CRISPR RNA (tracrRNA). In some aspects, the Cas protein to be used with the T cell targeted delivery system is a type I Cas protein. In some aspects, the Cas protein is a type II Cas protein. In some aspects, the type II Cas protein is Cas9. In some aspects, the type II Cas, e.g., Cas9 protein, is a human codon-optimized Cas.

In some aspects, the Cas protein is a “nickase” that can create single strand breaks (i.e., “nicks”) at the target site without cutting both strands of double stranded DNA (dsDNA). Cas9, for example, comprises two nuclease domains—a RuvC-like nuclease domain and an HNH-like nuclease domain—which are responsible for cleavage of opposite DNA strands. Mutation in either of these domains can create a nickase. Examples of mutations creating nickases can be found, for example, WO/2013/176772A1 and WO/2013/142578A1, each of which is incorporated by reference.

In some aspects, two separate Cas proteins (e.g., nickases) specific for a target site on each strand of dsDNA can create overhanging sequences complementary to overhanging sequences on another nucleic acid, or a separate region on the same nucleic acid. The overhanging ends created by contacting a nucleic acid with two nickases specific for target sites on both strands of dsDNA can be either 5′ or 3′ overhanging ends. For example, a first nickase can create a single strand break on the first strand of dsDNA, while a second nickase can create a single strand break on the second strand of dsDNA such that overhanging sequences are created. The target sites of each nickase creating the single strand break can be selected such that the overhanging end sequences created are complementary to overhanging end sequences on a different nucleic acid molecule. The complementary overhanging ends of the two different nucleic acid molecules can be annealed by the methods disclosed herein. In some aspects, the target site of the nickase on the first strand is different from the target site of the nickase on the second strand.

In some aspects, the first nucleic acid comprises a mutation that disrupts at least one amino acid residue of nuclease active sites in the Cas protein, wherein the mutant Cas protein generates a break in only one strand of the target DNA region, and wherein the mutation diminishes non-homologous recombination in the target DNA region. In some aspects, the first nucleic acid that encodes the Cas protein further comprises a nuclear localization signal (NLS). In some aspects, the nuclear localization signal is a SV40 nuclear localization signal.

Talen: In some aspects, the gene editing system used with the T cell targeted delivery system can comprise a TALEN system. TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze et al. (2010) Virulence 1:428-432; Christian et al. Genetics (2010) 186:757-761; Li et al. (2010) Nucl. Acids Res. (2010) doi:10.1093/nar/gkg704; and Miller et al. (2011) Nature Biotechnol. 29:143-148; all of which are herein incorporated by reference.

Examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference).

In some aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the methods and compositions provided herein include those that are specifically designed to bind at or near target nucleic acid sequences to be modified in a T cell using its targeting delivery system.

In some aspects, each monomer of the TALEN comprises 12-25 TAL repeats, wherein each TAL repeat binds a 1 bp subsite. In some aspects, the nuclease agent is a chimeric protein comprising a TAL repeat-based DNA binding domain operably linked to an independent nuclease. In some aspects, the independent nuclease is a FokI endonuclease. In some aspects, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by about 6 bp to about 40 bp cleavage site, and wherein the FokI nucleases dimerize and make a double strand break at a target sequence.

In some aspects, the nuclease agent of the T cell targeted delivery system comprises a first TAL-repeat-based DNA binding domain and a second TAL-repeat-based DNA binding domain, wherein the first and the second TAL-repeat-based DNA binding domains are operably linked to a FokI nuclease, wherein the first and the second TAL-repeat-based DNA binding domain recognize two contiguous target DNA sequences in each strand of the target DNA sequence separated by a 5 bp or 6 bp cleavage site, and wherein the FokI nucleases dimerize and make a double strand break.

Zinc-finger nucleases: In some aspects, the gene editing system of the T cell targeted delivery system can comprise a zinc-finger nuclease (ZFN) system. In some aspects, each monomer of the ZFN comprises 3 or more zinc finger-based DNA binding domains, wherein each zinc finger-based DNA binding domain binds to a 3 bp subsite. In some aspects, the ZFN is a chimeric protein comprising a zinc finger-based DNA binding domain operably linked to an independent nuclease. In some aspects, the independent endonuclease is a FokI endonuclease. In some aspects, the nuclease agent comprises a first ZFN and a second ZFN, wherein each of the first ZFN and the second ZFN is operably linked to a FokI nuclease, wherein the first and the second ZFN recognize two contiguous target DNA sequences in each strand of the target DNA sequence separated by about 6 bp to about 40 bp cleavage site or about a 5 bp to about 6 bp cleavage site, and wherein the FokI nucleases dimerize and make a double strand break. See, for example, US20060246567; US20080182332; US20020081614; US20030021776; WO/2002/057308A2; US20130123484; US20100291048; and, WO/2011/017293A2, each of which is herein incorporated by reference.

Meganucleases: In some aspects, the gene editing system of the T cell targeted delivery system can comprise a meganuclease system. Meganucleases (or homing endonucleases or HEases) have been classified into four families based on conserved sequence motifs, the families are the “LAGLIDADG,” “GIY-YIG,” “H—N—H,” and “His-Cys box” families. These motifs participate in the coordination of metal ions and hydrolysis of phosphodiester bonds.

HEases are notable for their long recognition sites, and for tolerating some sequence polymorphisms in their DNA substrates. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some aspects, a payload of the T cell targeted delivery system comprises a naturally occurring variant, and/or engineered derivative meganuclease. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used with the T cell targeted delivery system described herein, including, but not limited to, I-SceI, I-SceII, I-SceIII, I-SceIV, I-SceV, I-SecVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-Anil, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

In some aspects, the meganuclease recognizes double-stranded DNA sequences of 12 to 40 b.p. In some aspects, the meganuclease recognizes one perfectly matched target sequence in one of the heterologous plasmids described herein. In some aspects, the meganuclease is a homing nuclease, e.g., a “LAGLIDADG” family of homing nuclease such as I-SceI, I-CreI, and I-Dmol.

Restriction endonucleases: In some aspects, the gene editing system of the T cell targeted delivery system can comprise a restriction endonuclease, which includes Type I, Type II, Type III, and Type IV endonucleases. Type I and Type III restriction endonucleases recognize specific recognition sites, but typically cleave at a variable position from the nuclease-binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

I.B.ii Payload: Therapeutics mRNAs

In some aspects, the payload of the T cell targeted delivery system comprises an mRNA molecule, wherein the mRNA molecule is encapsulated within the LNP. The mRNA can be completely or partially encapsulated within the LNP. In some aspects, the payload comprises a single species of mRNA. In some aspects, the payload comprises one or more (e.g., a cocktail) mRNAs. mRNA may comprise at least one, two, three, four, five, six, seven, eight, nine, ten, or more modified nucleotides such as 2′OMe nucleotides. Preferably, uridine and/or guanosine nucleotides in the mRNA are modified with 2′OMe nucleotides. In some aspects, the mRNA may further comprise modified (e.g., 2′OMe-modified) adenosine and/or modified (e.g., 2′OMe-modified) cytosine nucleotides. In some aspects, the mRNA may further comprise, linkage modifications, e.g., phosphorothioate linkages.

In some aspects, the mRNA(s) are fully encapsulated in the LNP. With respect to formulations comprising an mRNA cocktail, the different types of mRNA species present in the cocktail (e.g., mRNA having different sequences) may be co-encapsulated in the same LNP, or each type of mRNA species present in the cocktail may be encapsulated in a separate LNP. The mRNA cocktail may be formulated in the LNP described herein using a mixture of two or more individual mRNAs (each having a unique sequence) at identical, similar, or different concentrations or molar ratios. In some aspects, a cocktail of mRNAs (corresponding to a plurality of mRNAs with different sequences) is formulated using identical, similar, or different concentrations or molar ratios of each mRNA species, and the different types of mRNAs are co-encapsulated in the same LNP. In some aspects, each type of mRNA species present in the cocktail is encapsulated in different LNP at identical, similar, or different mRNA concentrations or molar ratios, and the LNP thus formed (each containing a different mRNA payload) are administered separately (e.g., at different times in accordance with a therapeutic regimen), or are combined and administered together as a single unit dose (e.g., with a pharmaceutically acceptable carrier).

In some aspects, the mRNA comprises an mRNA vaccine, e.g., an mRNA vaccine to treat, e.g., COVID-19 (SARS-CoV2 infection), influenza, RSV infection, rabies, HPV infection, malaria, EBV infection, tuberculosis, CMV infection, Herpes zoster, Zika virus infection, HBV infection, yellow fever, PIV infection, hMPV infection, rotavirus infection, Nipah or virus infection. In some aspects, the mRNA comprises an mRNA encoding an antibody to treat, e.g., COVID-19, HIV infection, or Chikungunya virus infection. In some aspects, the mRNA encodes one or more components of gene editing system. In some aspects, the mRNA encodes a vaccine for the treatment of cancer, e.g., melanoma, NSCLC, cervical cancer, breast cancer, ovarian cancer, liver cancer, gastric cancer, pancreatic cancer, colorectal cancer, bladder cancer, prostate cancer, head and neck cancer, adenoidcystic carcinoma, cSCC, basal cell cancer, renal cell cancer, or AML, In some aspects, the vaccine for the treatment of cancer is a personal vaccine. In some aspects, the mRNA encodes a CAR (see below). In some aspects, the mRNA encodes an antibody or antigen-binding portion thereof. In some aspects, the mRNA encodes an antibody disclosed below or an antigen-binding portion thereof (e.g., the antigen-binding portion of a CAR). In some aspects, the mRNA encodes a protein for protein replacement therapy. In some aspects, the mRNA encodes a component of the CRISPR/Cas nuclease system.

In some aspects, the mRNA encodes a protein for protein replacement therapy in genetic diseases such as cystic fibrosis, propionic academia, methylmalonic academia, CSD1a, phenylketonuria, CN-1, OTC, or hemophilia. In some aspects, the mRNA encodes a protein for protein replacement therapy in autoimmune disorders. In some aspects, the mRNA encodes a protein for protein replacement therapy in metabolic disorders, e.g., type 2 diabetes. In some aspects, the mRNA encodes a protein for protein replacement therapy in cardiovascular disease, e.g., hypercholesterolemia or myocardial ischemia. In some aspects, the mRNA encodes a protein for protein replacement therapy in fibrosis, e.g., hypertrophic scarring, liver fibrosis, lung fibrosis, anemia, or primary sclerosing cholangitis. See Qin et al. (2022) Signal Transduction and Targeted Therapy 7:166; Huang et al. (2022) Nature Medicine 28:2273-2287; and Liu et al. (2022) Nature Reviews Cancer 23:526-543, which are herein incorporated by reference in their entireties.

### I.B.iii Payload: Chimeric Antigen Receptor (CARs)

In some aspects, the payload of the T cell targeted delivery system comprises a CAR or polynucleotide encoding a CAR. As used herein, the term “Chimeric Antigen Receptor” or alternatively a “CAR” refers to a recombinant polypeptide construct comprising at least (i) an extracellular antigen binding domain, (ii) a transmembrane domain, and (iii) a cytoplasmic signaling domain comprising a functional signaling domain derived from a stimulatory molecule and/or costimulatory molecule. In its simplest form, a CAR comprises a set of polypeptides, typically two, which when in an immune effector cell, provides the cell with specificity for a target cell, typically a cancer cell, and with intracellular signal generation. In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides include a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (CD3 zeta). In some aspects, the cytoplasmic signaling domain comprises a primary signaling domain (e.g., a primary signaling domain of CD3 zeta). In some aspects, the cytoplasmic signaling domain further comprises one or more functional signaling domains derived from at least one costimulatory molecule defined below. In some aspects, the costimulatory molecule is, e.g., 4-1BB, CD27, and/or CD28.

In some aspects, the CAR comprises a chimeric fusion protein comprising an antigen-binding domain (extracellular antigen binding domain), a transmembrane domain and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain (extracellular antigen binding domain) linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an extracellular antigen binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises an extracellular antigen binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

In some aspects, the present disclosure provide polynucleotides encoding a CAR comprising, e.g., (i) an extracellular antigen binding domain, (ii) a transmembrane domain, (iii) an intracellular domain, and (iv) a CAR spacer comprising an amino acid sequence derived from a human immunoglobulin (Ig) hinge region and/or loop region (i.e., a CAR spacer), and optionally a linker (e.g, a Gly-Ser rich linker) wherein the spacer is located between the extracellular antigen binding domain and the transmembrane domain. In some aspects, the present disclosure provides a recombinant nucleic acid construct comprising a transgene encoding a CAR of the present disclosure. The present disclosure also provides a CAR encoded by one or more of the polynucleotide sequences or the vectors disclosed herein. In some aspects, the CAR of present disclosure is designed as a standard CAR, a split CAR, an off-switch CAR, an on-switch CAR, a first-generation CAR, a second-generation CAR, a third-generation CAR, a fourth-generation CAR, or a fifth generation CAR.

In some aspects, the payload of the T cell targeted delivery system comprises a CAR or polynucleotide encoding a CAR wherein the CAR's antigen binding portion is an anti-CD19, anti-BCMA, anti-HER2, anti-CD20, anti-CD22, anti-IL13Ra2, anti-GPC3, or combination thereof. In some aspects, the payload comprises a CAR or polynucleotide encoding a CAR wherein the CAR's antigen binding portion comprises an scFv derived from a therapeutic antibody disclosed herein, e.g., a therapeutic antibody disclosed herein targeting an antigen expressed on the surface of T cells. In some aspects, the CAR is a monospecific or a bispecific CAR.

In some aspects, the payload of the T cell targeted delivery system comprises an anti-CD20 CAR. In some aspects, the anti-CD20 CAr is an RN105 CAR (SEQ ID NO: 542). In some aspects, the anti-CD20 CAR is an RN105 CAR encoded by an mRNA of SEQ ID NO: 543. In some aspects, the payload of the T cell targeted delivery system comprises an anti-CD79b CAR. In some aspects, the anti-CD79b CAR is an RN111 CAR (SEQ ID NO: 545). In some aspects, the anti-CD79b CAR is an RN111 CAR encoded by an mRNA of SEQ ID NO: 546. In some aspects, the payload of the T cell targeted delivery system comprises an anti-CD19 CAR. In some aspects, the anti-CD19 CAR is an RN068 CAR (SEQ ID NO: 548). In some aspects, the anti-CD19 CAR is an RN068 CAR encoded by an mRNA of SEQ ID NO: 549. In some aspects, the anti-CD19 is an RN082 CAR (SEQ ID NO: 551). In some aspects, the anti-CD19 CAR is an RN082 CAR encoded by an mRNA of SEQ ID NO: 552. In some aspects, the anti-CD19 is an RN083 CAR (SEQ ID NO: 554). In some aspects, the anti-CD19 CAR is an RN083 CAR encoded by an mRNA of SEQ ID NO: 555. In some aspects, the anti-CD19 is an RN084 CAR (SEQ ID NO: 557). In some aspects, the anti-CD19 CAR is an RN083 CAR encoded by an mRNA of SEQ ID NO: 558.

### CAR Structure

Although their fundamental modular structure has remained similar since their inception, CARs can be classified into five generations according to the organization of their intracellular signaling domain. Structural changes of CARs focus mostly on the intracellular region because CARs are designed based on the principles of TCR (T cell receptors) and costimulatory signaling. In these structures, the intracellular domains work as the functional endpoints by triggering differentiation, cytotoxic response, cytokine production, and by recruiting other immune cells that will enhance the process of tumor elimination. These mechanisms allow a non-MHC restricted targeting of tumors. For this reason, most strategies aiming to enhance CAR-T cell clinical efficacy have focused on amplifying and sustaining these signaling pathways.

First-generation CARs: First-generation CARs contain a single CD3 ζ− chain or FcεRIγ intracellular domain devoid of additional costimulatory domains. These complexes were very similar to endogenous TCR; however, they suffer from one major drawback—the inability to produce sufficient IL-2 (interleukin-2). Given their weak response, first-generation CARs have to be supplemented with exogenous IL-2 to ensure an efficient response. Moreover, studies revealed these modified cells still displayed low cell proliferation and short in vivo lifespan, which further prompted the development of costimulatory domains.

Second-generation CARs: Second-generation CARs attempt to solve the challenges caused by inadequate proliferation, low cytokine production, and the short lifespan of conventional CAR-T cells. They do this by leveraging the power of dual signaling known to drive strong T cell proliferation in natural systems. This new generation of CARs contains additional cytoplasmic domains such as CD28, 4-1BB, or OX-40, capable of delivering a secondary signal upon encountering a tumor antigen. Clinical and preclinical studies reveal that the presence of the costimulatory signal is able to improve proliferation, cytotoxicity, and sustained response due to longer in vivo half-lives. Studies also reveal that the composition of the costimulatory domain plays a vital role in modulating these parameters. For instance, 4-1BBζ-CAR-T cells might persist longer in circulation than CD28ζ-CAR-T. However, while the first may cause early exhaustion of CAR-T cells, the second was also reported to lead to the constitutive stimulation (activation in the absence of the antigen). For this reason, CAR design has evolved into better costimulatory constructs.

Third-generation CARs: Third-generation CARs have been made by combining multiple costimulatory signaling domains within the endodomain. Known examples of these constructs include CD3ζ-CD28-OX40 or CD3ζ-CD28-41BB. Although these have been used to treat successfully certain types of cancer with good safety profiles, increased persistence, and proliferation, no enhanced efficacy was achieved in comparison to second-generation CAR-T cells.

Fourth-generation CARs: Given that the presence of multiple costimulatory domains failed to improve CAR-T cell efficacy, fourth-generation CARs are based on second-generation constructs. The difference between the two generations is that the latest is additionally modified with a constitutive or inducible expression cassette containing a transgenic protein such as a cytokine. These are called T cell redirected for universal cytokine-mediated killing (TRUCK) CAR-T cells and they are designed to deliver the transgenic product to the targeted tumor site. This is usually achieved by engineering these cells to carry a nuclear factor of the activated T cell (NFAT)-responsive cassette (containing the transgenic cytokine such as IL-12). The expression of the transgene is thus induced when CD3ζ-containing CARs engage with their specific target. In practice, the engineering of TRUCK CAR-T cells requires the transfer of two transgenic cassettes—one for the CAR structure and another for the inducible cytokine. In preclinical models, the presence of a cytokine transgene greatly enhances the efficacy of CAR-T cell therapies in comparison to second-generation CARs. Moreover, the approach is also successful at avoiding systemic toxicity—one of the most common drawbacks of CAR-T cell therapy.

Fifth-generation CARs: CAR-T cell therapies have greatly evolved in an attempt to enhance persistence, proliferation, safety, and efficacy. However, it remains challenging to minimize CAR-T cells' off-target and off-tumor toxicity. The fifth generation differs from the previous ones because they integrate an additional membrane receptor. In TRUCKS or fourth-generation CARs, these modified T cells are activated by coming into contact with their target antigen, which leads to the induction of the secondary transgene, subsequent transcription, and secretion into the extracellular fluid. In this approach, the secreted signal not only stimulates CAR-T cells to remain active and form memory T cells but also reactivates the immune system to respond to restimulation. CAR-T cells that use membrane receptors (fifth generation) act according to a different principle. Several approaches can be used, e.g., the addition of IL-2 receptors that allows JAK/STAT pathway activation in an antigen-dependent manner, incorporation of a drug-dependent OFF-switch receptor leading to CAR depletion or an ON-switch receptor leading to activation. Based on these principles, lenalidomide-gated CARs were produced and tested. Although these cells proved to be slightly less efficacious in vitro, they were much more controllable than earlier generations of CARs resulting in a better safety profile and wider therapeutic window.

### CAR Architecture—Elements

Intracellular signaling domain: The “intracellular signaling domain” of a CAR refers to an intracellular portion of a CAR that acts by transmitting information within the cell to regulate cellular activity via defined signaling pathways by generating second messengers or functioning as effectors by responding to such messengers. Thus, the intracellular signaling domain can generate a signal that promotes an immune effector function of the CAR containing cell, e.g., a CART cell. Examples of immune effector function, e.g., in a CART cell, include cytolytic activity and helper activity, including the secretion of cytokines. In some aspects, the intracellular signal domain is the portion of the protein that transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

In some aspects, the intracellular signaling domain can comprise a primary intracellular signaling domain. Exemplary primary intracellular signaling domains include those derived from the molecules responsible for primary stimulation, or antigen dependent simulation. In some aspects, the intracellular signaling domain can comprise a costimulatory intracellular domain. Exemplary costimulatory intracellular signaling domains include those derived from molecules responsible for costimulatory signals, or antigen independent stimulation. For example, in the case of a CAR T, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif that is known as an Immunoreceptor Tyrosine-based Activation Motif or ITAM. Examples of ITAM containing primary cytoplasmic signaling sequences include, but are not limited to, those derived from CD3 zeta, FcR gamma, common FcR gamma (FCER1G), Fc gamma RIIa, FcR beta (Fc Epsilon Rib), CD3 gamma, CD3 delta, CD3 epsilon, CD5, CD22, CD79a, CD79b, CD278 (ICOS), FcεRI, CD66d, CD32, DAP10 and DAP12. In some aspects, the signaling domain is derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, or B7-H3. In some aspects, the intracellular signaling domain comprises a functional signaling domain of 4-1BB and/or a functional signaling domain of CD3 zeta. In some aspects, the intracellular signaling domain comprises a functional signaling domain of CD27 and/or a functional signaling domain of CD3 zeta.

Costimulatory domain: In some aspects, the CAR comprises a costimulatory domain comprising a functional signaling domain of a protein selected from the group consisting of OX40, CD2, CD27, CD28, CDS, ICAM-1, LFA-1 (CD11a/CD18), ICOS (CD278), and 4-1BB (CD137). In some aspects, the costimulatory domain comprises a functional signaling domain of a protein selected from the group consisting of MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In some aspects, the costimulatory domain comprises 4-1BB, CD27, CD28, or ICOS.

Transmembrane domain: In some aspects, the CAR comprises a transmembrane domain of a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137 and CD154. The transmembrane domain may be derived either from a natural or from a recombinant source. Where the source is natural, the domain may be derived from any membrane-bound or transmembrane protein. In some aspects, the transmembrane domain is capable of signaling to the intracellular domain(s) whenever the CAR of the present disclosure has bound to a target.

In some aspects, a transmembrane domain can include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

Extracellular antigen recognition domain: In some aspects, the CAR comprises an extracellular antigen recognition domain (antigen-binding domain) comprising an antibody disclosed herein or an antigen-binding portion thereof. In some aspects, the extracellular antigen recognition domain comprises a scFv derived from an antibody disclosed herein. In some aspects, the extracellular antigen recognition domain comprises an antibody in a format disclosed in the present application. The antigen-binding domain portion of a CAR, generally comprises an antibody or antibody fragment thereof that may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the antigen-binding domain of a CAR composition disclosed herein comprises an antibody fragment. In some aspects, the CAR comprises an antibody fragment that comprises a scFv. The term “scFv” refers to a fusion protein comprising at least one antibody portion comprising a variable region of a light chain and at least one antibody portion comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference. An scFv can comprise a linker of, e.g., at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some aspects, the linker sequence comprises amino acids glycine and serine. In some aspects, the linker sequence comprises sets of glycine and serine repeats such as (Gly₄Ser)n, where n is a positive integer equal to or greater than 1 (SEQ ID NO: 512). In some aspects, the linker can be (Gly₄Ser)₄ (SEQ ID NO: 513) or (Gly₄Ser)₃ (SEQ ID NO: 514), or any Gly-Ser rich linker. Variation in the linker length may retain or enhance activity, giving rise to superior efficacy in activity studies.

In some aspects, the antigen-binding portion of a CAR disclosed herein can comprise, consist, or consist essentially of an MSTAR, single domain antibody, maxibody, minibody, nanobody, intrabody, diabody, triabody, tetrabody, v-NAR and bis-scFv (see, e.g., Hollinger and Hudson, Nature Biotechnology 23:1126-1136, 2005). In some aspects, the antigen-binding portion of a CAR disclosed herein can comprise, consist, or consist essentially of one or more CDRs grafted into scaffolds based on polypeptides such as a fibronectin type III (Fn3)(see U.S. Pat. No. 6,703,199, which describes fibronectin polypeptide minibodies). Also included are antibody mimics based on the scaffold of the fibronectin type III domain (monobodies), other scaffolding systems (e.g., tenascin) in which one or more CDRs are grafted, aptamers, etc.

Also included are other suitable antigen-binding domains, e.g., VHH antibody, DARPin (designed ankyrin repeat proteins), affibody, monobody, adnectin, alphabody, Albumin-binding domain, Adhiron, Affilin and other gamma-B crystallin-derived artificial proteins, Affimer, Affitin (NANOFITIN™), Anticalin, Armadillo repeat proteins (ARM-repeat protein such as, e.g., β-catenin, α-importin, plakoglobin, adenomatous polyposis coli, ARMC4, ARMCX3, etc.), Atrimer (e.g., tetranectin and derived proteins), Avimer/Maxibody, Centyrin, Fynomer and other Fyn SH3 domain-derived proteins, Kunitz domain, Obody/OB-fold, Pronectin, Repebody, or any synthetic and/or computationally designed binding-protein or scaffold.

The modular architecture of antibodies has been exploited to create more than 60 different bispecific or multispecific antibody formats. Accordingly, in some aspects, the antibody can be in a format selected, e.g., from crossMab, DAF (Dual Action Fab) (two-in-one), DAF (four-in-one), DutaMab, DT-IgG, Knobs-in-holes common LC, Knobs-in-holes assembly, Charge pair, Fab-arm exchange, SEEDbody, Triomab, LUZ-Y (bispecific antibody with a leucine zipper inducing heterodimerization of two HCs), Fcab, KX-body, Orthogonal Fab, DVD-IgG (dual variable domain IgG), IgG(H)-scFv, scFv-(H)IgG, IgG(L)-scFv, scFv-(L)IgG, IgG(L,H)-Fv, IgG(H)—V, V(H)—IgG, IgG(L)-V, V(L)-IgG, KIH IgG-scFab, 2scFv-IgG, IgG-2scFv, scFv4-Ig, Zybody, DVI-IgG (four-in-one), Nanobody, Nanobody-HSA, BiTE (bispecific T cell engager), Diabody, DART (dual-affinity-retargeting), TandAb (tandem antibody), scDiabody, scDiabody-CH3, Triple Body, Miniantibody, Minibody, TriBi minibody, scFv-CH3 KIH, Fab-scFv, scFv-CH-CL-scFv, F(ab′)2, F(ab′)2-ScFv2, scFv-KIH, Fab-scFv-Fc, Tetravalent HC Ab, scDiabody-Fc, Diabody-Fc, Tandem scFv-Fc, Intrabody, Dock and Locck, ImmTAC, HSAbody, scDiabody-HSA, Tandem scFv-Toxin, IgG-IgG, Cov-X-Body, and scFv1-PEG-scFV2. In some aspects, the CAR comprises an MSTAR antibody.

In some aspects, the extracellular antigen recognition domain specifically binds to an antigen, e.g., a tumor antigen, selected from the selected from the group consisting of ROR1, HER2, AFP, CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY—BR-1, UPK2, HAVCR1, ADRB3, PANX3, GPR20, LY6K, OR51E2, TARP, WTl, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, and any combinations thereof.

CAR spacer: The term “CAR spacer” as used herein refers to a polypeptide sequence that is capable of covalently linking together two spaced moieties: an extracellular antigen recognition domain, and the transmembrane domain of the CAR. In some aspects, the CAR spacer comprises an amino acid sequence derived from a human immunoglobulin (Ig) hinge region and/or loop region, and optionally a linker (e.g, a gly-ser rich linker) wherein the spacer is located between the antigen-binding domain and the transmembrane domain. In some aspects, the CAR spacer is a spacer disclosed in U.S. Appl. Publ. No. 2021-0380658, which is herein incorporated by reference in its entirety.

In some aspects, the CAR spacer comprises an amino acid sequence derived from a hinge region located between the CH1 and CH2 constant domains of a human immunoglobulin, e.g., IgA1, IgA2, IgG1, IgG2, IgG3, IgG4, IgD, IgE, or IgM, and optionally one or more amino acids from an adjacent CH1 and/or CH2 domain, or a combination thereof (e.g., several concatenated hinge region derived CAR spacer). In some aspects, the CAR spacer comprising an amino acid sequence derived from a loop region of a constant domain of a human immunoglobulin, e.g., IgA1, IgA2, IgG1, IgG2, IgG3, IgG4, IgD, IgE, or IgM, and optionally one or more amino acids from an adjacent β-strand, or a combination thereof (e.g., several concatenated loop region derived CAR spacers).

In some aspects, the CAR spacer comprises a subsequence of an immunoglobulin heavy chain selected the group consisting of human IgA1 (Uniprot: P01876, IGHA1_HUMAN, immunoglobulin heavy constant alpha 1), human IgA2 (Uniprot P01877, IGHA2_HUMAN, immunoglobulin heavy constant alpha 2), murine IgG2A (Uniprot P01665, GCAM_MOUSE, immunoglobulin gamma 2A chain C region), human IgG1 (Uniprot P01857, IGHG1_HUMAN, immunoglobulin heavy constant gamma 1), human IgG2 (Uniprot P01859, IGHG2_HUMAN, immunoglobulin heavy constant gamma 2), human IgG3 (Uniprot P01860, IGHG3_HUMAN, immunoglobulin heavy constant gamma 3), human IgG4 (Uniprot P01861, IGHG4, immunoglobulin heavy constant gamma 4), human IgD (Uniprot P01880, IGHD_HUMAN, immunoglobulin heavy constant delta), human IgE (Uniprot P01854, IGHE_HUMAN, immunoglobulin heavy constant chain epsilon), or IgM (Uniprot P01871, IGHM_UMAN, immunoglobulin heavy constant mu), wherein the subsequence comprises the CH1-CH2 hinge region or a portion thereof. In some aspects, the subsequence further comprises an adjacent portion of a CH1 and/or CH2 constant domain.

In some aspects, the CAR spacer comprises a subsequence of an immunoglobulin heavy chain selected the group consisting of human IgA1 (Uniprot: P01876, IGHA1_HUMAN, immunoglobulin heavy constant alpha 1), human IgA2 (Uniprot P01877, IGHA2_HUMAN, immunoglobulin heavy constant alpha 2), murine IgG2A (Uniprot P01665, GCAM_MOUSE, immunoglobulin gamma 2A chain C region), human IgG1 (Uniprot P01857, IGHG1_HUMAN, immunoglobulin heavy constant gamma 1), human IgG2 (Uniprot P01859, IGHG2_HUMAN, immunoglobulin heavy constant gamma 2), human IgG3 (Uniprot P01860, IGHG3_HUMAN, immunoglobulin heavy constant gamma 3), human IgG4 (Uniprot P01861, IGHG4, immunoglobulin heavy constant gamma 4), human IgD (Uniprot P01880, IGHD_HUMAN, immunoglobulin heavy constant delta), human IgE (Uniprot P01854, IGHE_HUMAN, immunoglobulin heavy constant chain epsilon), or IgM (Uniprot P01871, IGHM_UMAN, immunoglobulin heavy constant mu), wherein the subsequence comprises a loop region from a constant domain or a portion thereof. In some aspects, the subsequence further comprises an adjacent portion of a β-strand.

### Bispecific CARs

In some aspects, the CARs of the present disclosure are bispecific CARs. Accordingly, in some aspects, the polynucleotide encoding a CAR of the present disclosure encodes at least a polypeptide of a bispecific CAR (e.g., a CAR targeting a first antigen and second antigen). In some aspects, the antigen-binding domain of a CAR of the present disclosure is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some aspects, the first and second epitopes are on the same antigen, e.g., the same protein (or subunit of a multimeric protein). In some aspects, the first and second epitopes overlap. In some aspects, the first and second epitopes do not overlap. In some aspects, the first and second epitopes are on different antigens, e.g., different proteins (or different subunits of a multimeric protein).

In some aspects, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some aspects, a bispecific antibody molecule comprises a half antibody having binding specificity for a first epitope and a half antibody having binding specificity for a second epitope. In some aspects, a bispecific antibody molecule comprises a half antibody, or fragment thereof, having binding specificity for a first epitope and a half antibody, or fragment thereof, having binding specificity for a second epitope. In some aspects, a bispecific antibody molecule comprises a scFv, or fragment thereof, have binding specificity for a first epitope and a scFv, or fragment thereof, have binding specificity for a second epitope.

In some aspects, the antibody molecule is a multi-specific (e.g., a bispecific or a trispecific) antibody molecule. Protocols for generating bispecific or heterodimeric antibody molecules are known in the art.

Within each antibody or antigen-binding antibody fragment (e.g., scFv) of a bispecific antibody molecule, the VH can be upstream or downstream of the VL. In some aspects, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In some aspects, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁-VH₁—VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁—VH₂-VL₂. In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some aspects, a bispecific CAR comprises VLs, VHs, and optionally one or more linkers in an arrangement as described herein.

### Inducible CARs

In some aspects, the CAR of the present disclosure is regulated by a constitutive promoter, e.g., immediate early cytomegalovirus (CMV) promoter, Elongation Growth Factor-la (EF-1α), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. However, the regulation of the expression of a CAR of the present disclosure is not limited to the use of a constitutive promoter. Thus, in some aspects, the CAR of the present disclosure encoded by a polynucleotide disclosed herein is an inducible CAR. The term “inducible” refers to the presence of an “inducible promoter,” i.e., a nucleotide sequence which, when operably linked with a polynucleotide which encodes or specifies a gene product, e.g., a CAR of the present disclosure, causes the gene product to be produced in a cell substantially only when an inducer which corresponds to the promoter is present in the cell. The use of an inducible promoter provides a molecular switch capable of turning on expression of the polynucleotide sequence that it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some aspects, a polynucleotide encoding a CAR of the present disclosure comprises a “tissue-specific” promoter, i.e., a nucleotide sequence which, when operably linked with a polynucleotide which encodes or specifies a gene product, e.g., a CAR of the present disclosure, causes the gene product to be produced in a cell substantially only if the cell is a cell of the tissue type corresponding to the promoter.

### CAR Sequences

In some aspects, the CAR targets CD19. In some aspects, the CAR targets CD20. In some aspects, the CAR targets CD19 and CD3E. In some aspects, the CAR is acmucabtagene autoleucel (INN/IMGT No. 11832) an anti-CD19/anti-CD3E CAR for the treatment of B cell lymphoma. In some aspects, the CAR is anbalcabtagene autoleucel (INN/IMGT No. 12186) an anti-CD19 CAR for the treatment of diffuse large B cell lymphoma (DLBCL). In some aspects, the CAR is azercabtagene zapreleucel (INN/IMGT No. 11438) an anti-CD19 CAR for the treatment of Non-Hodgkin's lymphoma (NHL). In some aspects, the CAR is axicabtagene ciloleucel (YESCARTA®) (INN/IMGT No. 10518), an anti-CD19 CAR for the treatment of diffuse large B-cell lymphoma and follicular lymphoma. In some aspects, the CAR is brexucabtagene autoleucel (TECARTUS®) (INN/IMGT No. 11886) an anti-CD19 CAR for the treatment of mantle cell lymphoma and B-cell precursor ALL. In some aspects, the CAR is cemacabtagene ansegedleucel (INN/IMGT No. 12556) an anti-CD19 CAR. In some aspects, the CAR is evoncabtagene pazurgedleucel (INN/IMGT No. 11599) an anti-CD19 CAR for the treatment of hematologic-blood cancer. In some aspects, the CAR is inaticabtagene autoleucel (INN/IMGT No. 12418) an anti-CD19 CAR. In some aspects, the CAR is lisocabtagene maraleucel (BREYANZI®) (INN/IMGT No. 10805) an anti-CD19 CAR for the treatment of diffuse large B-cell lymphoma. In some aspects, the CAR is obecabtagene autoleucel (INN/IMGT No. 11486) an anti-CD19 CAR for the treatment of acute lymphocytic leukemia (ALL). In some aspects, the CAR is tisagenlecleucel (KYMRIAH®) (INN/IMGT No. 10557), an anti-CD19 CAR for the treatment of B-cell precursor ALL, diffuse large B-cell lymphoma, or follicular lymphoma. In some aspects, the CAR is idecabtagene vicleucel (ABECMA®) (INN/IMGT No. 10906) an anti-BCMA CAR for the treatment of multiple myeloma. In some aspects, the CAR is ciltacabtagene autoleucel (CARVYKTI®) (INN/IMGT No. 11131) an anti-BCMA CAR for the treatment of multiple myeloma.

In some aspects, the CAR is selected from the group consisting of xicabtagene ciloleucel (KTE-C19), vadacabtagene leraleucel (JCAR015), tisagenlecleucel (CTL019, CART19), lisocabtagene maraleucel (JCAR017), olitresgene autoleucel, letetresgene autoleucel, idecabtagene vicleucel (bb-2121), mipetresgene autoleucel, tebrocabtagene autoleucel (TBI-1501), ciltacabtagene autoleucel (JNJ-68284528, LCAR-B38M), orvacabtagene autoleucel (FCARH-143, JCARH-125), afamitresgene autoleucel (MAGEA4c1032T), relmacabtagene autoleucel (JWCAR029), gavocabtagene autoleucel (TC-210), azercabtagene zapreleucel (JWCAR029), obecabtagene autoleucel (AUTO-1, CAT-41BBZ, CD19CAT-41BBZ), tacatresgene autoleucel, zamtocabtagene autoleucel (MB-CART2019.1), evoncabtagene pazurgedleucel (CTX-110), zevorcabtagene, autoleucel (CT053, CT053, CAR-BCMA), motacabtagene lurevgedleucel (CTX120), acmucabtagene autoleucel (TAC01-CD19), brexucabtagene autoleucel (KTE-X19), itezocabtagene autoleucel (CD30-CAR-T), plixacabtagene autoleucel, rapcabtagene autoleucel, volamcabtagene durzigedleucel, equecabtagene autoleucel, anbalcabtagene autoleucel, varnimcabtagene autoleucel, satricabtagene autoleucel, pomlucabtagene autoleucel, inaticabtagene autoleucel, anitocabtagene autoleucel, durcabtagene autoleucel, tinocabtagene autoleucel, besvatresgene autoleucel, suvutresgene autoleucel, cemacabtagene ansegedleucel, trovocabtagene autoleucel, prizloncabtagene autoleucel, ribrecabtagene autoleucel, fencabtagene autoleucel, and firicabtagene autoleucel.

In some aspects, the CAR is a CAR disclosed in TABLE 1. In some aspects, the CAR comprises a VH and/or VL disclosed in TABLE 1. In some aspects, the CAR comprises the spacer, transmembrane, and cytoplasmic region of a CAR disclosed in TABLE 1 with the VH and VL regions of said CAR arranged in an MSTAR format.

IMGT Acc. Nos. in TABLE 1 refer to the INN number for each CAR construct as disclosed in the release of the International Immunogenetics Information System database (IMGT database, available at www dot imgt dot org) publicly available on Dec. 6, 2023. The information corresponding to the IMGT/2Dstructure-DB card for each IMGT Acc No. disclosed herein is incorporated by reference in its entirety.

TABLE 1
Chimeric antigen receptors (CARs). The structure of each CAR corresponds to its IMGT chain description. For example, V-
KAPPA (1-106) [D1] + VH (127-245) [D2] + V-KAPPA (282-388) [D3] + VH (404-525) [D4]
+ C-LIKE (535-548) [D5], indicates that the CAR has 5 domains, D1 to D5, which would include a C-like domain
(D5) and two scFvs VLκ-VH corresponding to D1-D2 (first scFV) and D3-D4 (second scFv). The pairs of numbers between
parentheses indicate amino acid positions, e.g., V-Kappa (1-106) of IMGT Acc. No. 11832 would be a V-Kappa domain comprising
amino acids 1-106 of acmucabtagene autoleucel, i.e., the amino acid subsequence comprising amino acids 1-106 of SEQ ID
NO: 299. The binding domains of the CARs disclosed in TABLE 1 can be replaced with a binding sequence derived from an
antibody disclosed in TABLE 2, e.g., an scFv or MSTAR antibody, or a combination thereof. An alternative binding domain
can be appended to the N-terminus of the sequences in the column labeled (“Non-binding portion”).

| | | | | Full CAR | Non-binding |
|---|---|---|---|---|---|
| IMGT | | | | sequence | portion |
| Acc. No. | Name | Target | Structure | SEQ ID NO | SEQ ID NO |
| 11832 | acmucabtagene | Homo | V-KAPPA (1-106) [D1] + VH (127-245) [D2] + V- | 299 | 473 |
| | autoleucel | sapiens | KAPPA (282-388) [D3] + VH (404-525) [D4] + C-LIKE | | |
| | | CD19/CD3E | (535-548) [D5] | | |
| 12186 | anbalcabtagene | Homo | V-KAPPA (1-106) [D1] + VH (124-242) [D2] + | 300 | 474 |
| | autoleucel | sapiens | TRANSMEMBRANE-REGION (243-311) [D3] + | | |
| | | CD19 | TNFRSF9 (Pr214-255) (312-353) [D4] + CD247 (Pr52- | | |
| | | | 164) (354-465) [D5] | | |
| 10518 | axicabtagene | Homo | V-KAPPA (1-106) [D1] + VH (126-245) [D2] + CD28 | 301 | 475 |
| | ciloleucel | sapiens | (Pr114-220) (249-355) [D3] + CD247 (Pr52-164) (356- | | |
| | YESCARTA ® | CD19 | 467) [D4] | | |
| 11438 | azercabtagene | Homo | V-KAPPA (1-106) [D1] + VH (124-242) [D2] + | 302 | 476 |
| | zapreleucel) | sapiens | TRANSMEMBRANE-REGION (243-311) [D3] + | | |
| | | CD19 | TNFRSF9 (Pr214-255) (334-353) [D4] + CD247 (Pr52- | | |
| | | | 164) (354-465) [D5] | | |
| 11886 | brexucabtagene | Homo | V-KAPPA (1-106) [D1] + VH (127-245) [D2] + CD28 | 303 | 477 |
| | autoleucel | sapiens | (Pr114-220) (249-355) [D3] + CD247 (Pr52-164) (356- | | |
| | TECARTUS ™ | CD19 | 467) [D4] | | |
| 12556 | cemacabtagene | Homo | VH (1-121) [D1] + V-KAPPA (137-248) [D2] + | 304 | 478 |
| | ansegedleucel | sapiens | TRANSMEMBRANE-REGION (252-321) [D3] + | | |
| | | CD19 | TNFRSF9 (Pr214-255) (322-363) [D4] + CD247 (Pr52- | | |
| | | | 164) (364-475) [D5] | | |
| 11599 | evoncabtagene | Homo | V-KAPPA (1-106) [D1] + VH (127-245) [D2] + CD8A | 305 | 479 |
| | pazurgedleucel | sapiens | (257-329) [D3] + CD28 (Pr114-220) (332-372) [D4] + | | |
| | | CD19 | CD247 (Pr52-164) (373-484) [D5] | | |
| 12418 | inaticabtagene | Homo | V-KAPPA (1-107) [D1] + VH (124-245) [D2] + | 306 | 480 |
| | autoleucel | sapiens | TRANSMEMBRANE-REGION (248-316) [D3] + | | |
| | | CD19 | TNFRSF9 (Pr214-255) (317-358) [D4] + CD247 (Pr52- | | |
| | | | 164) (359-470) [D5] | | |
| 10805 | lisocabtagene | Homo | V-KAPPA (1-106) [D1] + VH (127-245) [D2] + CD28 | 307 | 481 |
| | maraleucel | sapiens | (Pr153-179) (259-285) [D3] + TNFRSF9 (Pr214-255) | | |
| | BREYANZI ™ | CD19 | (286-327) [D4] + CD247 (Pr52-164) (328-439) [D5] + | | |
| | | | EGFR (Pr28-459) (486-657) [D6] | | |
| 11486 | obecabtagene | Homo | VH (1-119) [D1] + V-KAPPA (135-240) [D2] + | 308 | 482 |
| | autoleucel | sapiens | TRANSMEMBRANE-REGION (244-313) [D3] + | | |
| | | CD19 | TNFRSF9 (Pr214-255) (314-355) [D4] + CD247 (Pr52- | | |
| | | | 164) (356-406) [D5] | | |
| 12425 | anitocabtagene | Homo | THB (1-72) [D1] + TRANSMEMBRANE-REGION (84- | 309 | 483 |
| | autoleucel | sapiens | 152) [D2] + TNFRSF9 (Pr214-255) (153-194) [D3] + | | |
| | | TNFRSF17 | CD247 (Pr52-164) (195-306) [D4] | | |
| | | (BCM, BCMA, | | | |
| | | CD269) | | | |
| 11131 | ciltacabtagene | Homo | VH (1-119) [1] + VH (125-242) [2] + | 310 | 484 |
| | autoleucel | sapiens | TRANSMEMBRANE-REGION (245-324) [D3] + | | |
| | | TNFRSF17 | TNFRSF9 (Pr214-255) (325-355) [D4] + CD247 (Pr52- | | |
| | | (BCM, BCMA, | 164) (356-467) [D5] | | |
| | | CD269) | | | |
| 12119 | dalucabtagene | Homo | | 311 | 485 |
| | autoleucel | sapiens | | | |
| | | CD19/CD22 | | | |
| | | bispecific | | | |
| | | CAR | | | |
| 12461 | durcabtagene | Homo | VH (1-123) [D1] + V-LAMBDA (139-249) [D2] + | 312 | 486 |
| | autoleucel | sapiens | TRANSMEMBRANE-REGION (250-318) [D3] + | | |
| | | TNFRSF17 | TNFRSF9 (Pr214-255) (319-360) [D4] + CD247 (Pr52- | | |
| | | (BCM, BCMA, | 164) (361-472) [D5] | | |
| | | CD269) | | | |
| 12142 | equecabtagene | Homo | V-KAPPA (1-106) [D1] + VH (125-243) [D2] + | 313 | 487 |
| | autoleucel | sapiens | TRANSMEMBRANE-REGION (252-324) [D3] + | | |
| | | TNFRSF17 | TNFRSF9 (Pr214-255) (327-368) [D4] + CD247 (Pr52- | | |
| | | (BCM, BCMA, | 164) (369-480) [D5] | | |
| | | CD269) | | | |
| 12821 | fencabtagene | Homo | ANK-REPEAT (1-128) [D1] + V-KAPPA (165-271) [D2] + | 314 | 488 |
| | autoleucel | sapiens | VH (287-408) [D3] + C-LIKE (418-431) [D4] + | | |
| | | CD3E | CYTOPLASMIC-REGION (432-501) [D5] | | |
| | | Homo | | | |
| | | sapiens | | | |
| | | ERBB2 | | | |
| 12829 | firicabtagene | Homo | VH (1-124) [D1] + V-KAPPA (130-236) [D2] + | 315 | 489 |
| | autoleucel | sapiens | TRANSMEMBRANE-REGION (240-308) [D3] | | |
| | | CD22 | TNFRSF9 (Pr214-255) (309-350) [D4] + CD247 (Pr52- | | |
| | | | 164) (351-462) [D5] | | |
| 11437 | gavocabtagene | Homo | VH (1-111) [D1] + CD3E C-LIKE (132-226) [D2] + | 316 | 490 |
| | autoleucel | sapiens | CD3E CO (227-241) [D3] + CD3E TM (242-257) [D4] + | | |
| | | MSLN | CD3E CY (258-316) [D5] | | |
| 10906 | idecabtagene | Homo | V-KAPPA (1-111) [D1] + VH (130-246) [D2] + CD8A | 317 | 491 |
| | vicleucel | sapiens | (250-318) [D3] + TNFRSF9 (Pr214-255) (319-360) [D4] + | | |
| | | TNFRSF17 | CD247 (Pr52-164) (361-472) [D5] | | |
| 11887 | itezocabtagene | Homo | VH (1-122) [D1] + V-KAPPA (143-245) [D2] + CH2 | 318 | 492 |
| | autoleucel | sapiens | (266-375) [D3] + CH3 (376-480) [D4] + CD28 (Pr114- | | |
| | | TNFRSF8 | 220) (487-554) [D5] + CD247 (Pr52-164) (555-666) [D6] | | |
| 11721 | motacabtagene | Homo | VH (1-118) [D1] + V-KAPPA (134-245) [D2] + | 319 | 403 |
| | lurevgedleucel | sapiens | TRANSMEMBRANE-REGION (258-330) [D3] + | | |
| | | TNFRSF17 | TNFRSF9 (Pr214-255) (334-375) [D4] + CD247 (Pr52- | | |
| | | | 164) (376-487) [D5] | | |
| 11486 | obecabtagene | Homo | VH (1-119) [D1] + V-KAPPA (135-240) [D2] + | 320 | 494 |
| | autoleucel | sapiens | TRANSMEMBRANE-REGION (244-313) [D3] + | | |
| | | CD19 | TNFRSF9 (Pr214-255) (314-355) [D4] + CD247 (Pr52- | | |
| | | | 164) (356-406) [D5] | | |
| 11274 | orvacabtagene | Homo | V-LAMBDA (1-104) [D1] + VH (127-244) [D2] + CH2 | 321 | 495 |
| | autoleucel | sapiens | (257-365) [D3] + CH3 (366-470) [D4] + CD28 (Pr114- | | |
| | | TNFRSF17 | 220) (472-509) [D5] + TNFRSF9 (Pr214-255) (510-542) | | |
| | | | [D6] + CD247 (Pr52-164) (543-654) [D7] + EGFR1 (701- | | |
| | | | 1035) [D8] | | |
| 11931 | plixacabtagene | Homo | V-KAPPA (1-106) [D1] + VH (124-242) [D2] + CD3E C- | 322 | 496 |
| | autoleucel | sapiens | LIKE (263-357) [D3] + CD3E CO (358-372) [D4] + CD3E | | |
| | | CD19 | TM (373-388) [D5] + CD3E CY (389-447) [D6] | | |
| 12254 | pomlucabtagene | Homo | V-KAPPA (2-106) [D1] + VH (122-241) [D2] + | 323 | 497 |
| | autoleucel | sapiens | TRANSMEMBRANE-REGION (253-328) [D3] + CD28 | | |
| | | ganglioside | (Pr114-220) (329-368) [D4] + TNFRSF9 (Pr214-255) | | |
| | | GM2 | (374-415) [D5] + CD247 (Pr52-164) (416-527) [D6] | | |
| 12614 | prizloncabtagene | Homo | V-KAPPA (1-107) [D1] + VH (126-247) [D2] + VH (254- | 324 | 498 |
| | autoleucel | sapiens | 372) [D3] + V-KAPPA (391-496) [D4] + CD28 (Pr153- | | |
| | | CD19 | 179) (511-537) [D5] + TNFRSF9 (Pr214-255) (538-579) | | |
| | | Homo | [D6] + CD247 (Pr52-164) (580-691) [D7] | | |
| | | sapiens | | | |
| | | MS4A1 | | | |
| | | bispecific | | | |
| 11974 | rapcabtagene | Homo | V-KAPPA (1-106) [D1] + VH (124-242) [D2] + | 325 | 499 |
| | autoleucel | sapiens | TRANSMEMBRANE-REGION (243-311) [D3] | | |
| | | CD19 | TNFRSF9 (Pr214-255) (312-353) [D4] + CD247 (Pr52- | | |
| | | | 164) (354-465) [D5] | | |
| 11372 | relmacabtagene | Homo | V-KAPPA (1-106) [D1] + VH (127-245) [D2] + CD28 | 326 | 500 |
| | autoleucel | sapiens | (Pr153-179) (259-285) [D3] + TNFRSF9 (Pr214-255) | | |
| | | CD19 | (286-327) [D4] + CD247 (Pr52-164) (328-439) [D5] + | | |
| | | | EGFR1 (486-820) [D6] | | |
| 12662 | ribrecabtagene | Homo | VH (1-120) [D1] + TRANSMEMBRANE-REGION (123- | 327 | 501 |
| | autoleucel | sapiens | 193) [D2] + TNFRSF9 (Pr214-255) (194-235) [D3] + | | |
| | | TNFRSF17 | CD247 (Pr52-164) (236-348) [D4] | | |
| 12215 | satricabtagene | Homo | VH (1-118) [D1] + V-KAPPA (134-246) [D2] + | 328 | 502 |
| | autoleucel | sapiens | TRANSMEMBRANE-REGION (248-292) [D3] + CD28 | | |
| | | CLDN18 | (Pr114-220) (293-360) [D4] + CD247 (Pr52-164) (361- | | |
| | | | 473) [D5] | | |
| 11110 | tebrocabtagene | Homo | V-KAPPA (1-106) [D1] + VH (127-245) [D2] + CD28 | 329 | 503 |
| | autoleucel | sapiens | (Pr114-220) (249-355) [D3] + CD247 (Pr52-164) (356- | | |
| | | CD19 | 467) [D4] | | |
| 12483 | tinocabtagene | Homo | VH (1-119) [D1] + V-LAMBDA (125-234) [D2] + VH | 330 | 504 |
| | autoleucel | sapiens | (254-374) [D3] + V-KAPPA (380-487) [D4] + | | |
| | | CD19 | TRANSMEMBRANE-REGION (496-568) [D5] + | | |
| | | Homo | TNFRSF9 (Pr214-255) (571-612) [D6] + CD247 (Pr52- | | |
| | | sapiens | 164) (613-724) [D7] | | |
| | | MS4A1 | | | |
| | | bispecific | | | |
| 12557 | trovocabtagene | Homo | V-KAPPA (1-107) [D1] + VH (123-243) [D2] + | 331 | 505 |
| | autoleucel | sapiens | TRANSMEMBRANE-REGION (252-322) [D3] + | | |
| | | TNFRSF17 | TNFRSF9 (Pr214-255) (323-364) [D4] + CD247 (Pr52- | | |
| | | | 164) (365-476) [D5] | | |
| 10522 | vadacabtagene | Homo | VH (1-122) [D1] + V-KAPPA (138-244) [D2] + CD28 | 332 | 506 |
| | leraleucel | sapiens | (Pr114-220) (249-355) [D3] + CD247 (Pr52-164) (356- | | |
| | | CD19 | 467) [D4] | | |
| 12208 | varnimcabtagene | Homo | V-KAPPA (1-111) [D1] + VH (134-255) [D2] + | 333 | 507 |
| | autoleucel | sapiens | TRANSMEMBRANE-REGION (256-324) [D3] + | | |
| | | CD19 | TNFRSF9 (Pr214-255) (325-366) [D4] + CD247 (Pr52- | | |
| | | | 164) (367-479) [D5] | | |
| 12102 | volamcabtagene | Homo | VH (1-118) [D1] + V-KAPPA (135-245) [D2] + | 334 | 508 |
| | durzigedleucel | sapiens | TRANSMEMBRANE-REGION (258-330) [D3] + | | |
| | | CD70 | TNFRSF9 (Pr214-255) (334-375) [D4] + CD247 (Pr52- | | |
| | | | 164) (376-487) [D5 | | |
| 11574 | zamtocabtagene | Homo | VH (1-122) [D1] + V-KAPPA (124-228) [D2] + V- | 335 | |
| | autoleucel | sapiens | KAPPA (229-335) [D3] + VH (336-454) [D4] | | |
| | | CD19 | | | |
| | | Homo | | | |
| | | sapiens | | | |
| | | MS4A1 | | | |
| | | bispecific | | | |
| 11704 | zevorcabtagene | Homo | VH (1-119) [D1] + V-KAPPA (135-243) [D2] + | 336 | 509 |
| | autoleucel | sapiens | TRANSMEMBRANE-REGION (245-313) [D3] + | | |
| | | TNFRSF17 | TNFRSF9 (Pr214-255) (314-355) [D4] + CD247 (Pr52- | | |
| | | | 164) (356-468) [D5] | | |
| 11286 | afamitresgene | Homo | V-ALPHA (1-114) [D1] + C-ALPHA (116-205) [D2] + V- | 337 | 510 |
| | autoleucel | sapiens | BETA (305-417) [D3] + C-BETA-2 (418-546) [D4] | | |
| | | MAGEA4 | | | |
| 12537 | besvatresgene | Homo | V-ALPHA (1-114) [D1] + C-ALPHA (116-205) [D2] + V- | 338 | |
| | autoleucel | sapiens | BETA (305-417) [D3] + C-BETA-2 (418-546) [D4] | | |
| | | CTAG1B | | | |
| | | Homo | | | |
| | | sapiens | | | |
| | | CTAG2 | | | |
| 10866 | letetresgene | Homo | V-ALPHA (1-114) [D1] + C-ALPHA (116-205) [D2] + V- | 339 | |
| | autoleucel | sapiens | BETA (305-417) [D3] + C-BETA-2 (418-546) [D4] | | |
| | | CTAG1B | | | |
| 11109 | mipetresgene | Homo | V-BETA (1-113) [D1] + C-BETA-2 (114-242) [D2] + V- | 340 | |
| | autoleucel | sapiens | ALPHA (335-448) [D3] + C-ALPHA (450-539) [D4] | | |
| | | CTAG1B | | | |
| 10865 | olitresgene | Homo | V-ALPHA (1-111) [D1] + C-ALPHA (113-202) [D2] + V- | 341 | |
| | autoleucel | sapiens | BETA (302-411) [D3] + C-BETA-2 (412-540) [D4] | | |
| | | MAGEA10 | | | |
| 12538 | suvutresgene | Homo | V-ALPHA (1-114) [D1] + C-ALPHA (116-205) [D2] + V- | 342 | |
| | autoleucel | sapiens | BETA (305-417) [D3] + C-BETA-2 (418-546) [D4] | | |
| | | CTAG1B | | | |
| | | Homo | | | |
| | | sapiens | | | |
| | | CTAG2 | | | |
| 11534 | tacatresgene | Homo | C-BETA-2 (1-127) [D1] + C-ALPHA (231-254) [D2] | 343 | |
| | autoleucel | sapiens | | | |
| | | p/MH | | | |
| 10557 | tisagenlecleucel | Homo | V-KAPPA (1-106) [D1] + VH (124-242) [D2] + CD8A | 344 | 511 |
| | | sapiens | (243-311) [D3] + TNFRSF9 (Pr214-255) (312-353) [D4] + | | |
| | | CD19 | CD247 (Pr52-164) (354-465) [D5] | | |

In some aspects, a CAR of the present disclosure comprises an antigen-binding domain derived from a CD19 and/or CD20 antibody disclosed in TABLE 2, e.g., an scFv, tandem scFv, or MSTAR antibody, covalently linked to a sequence set forth in SEQ ID NOS: 473 to 511.

In some aspects, a CAR of the present disclosure comprises an antigen-binding domain comprising an antibody or a binding sequence derived from an antibody or more (e.g., in the case of a bispecific binding sequence), for example, an scFv (for example, a monospecific or bispecific scFv), tandem scFv (e.g., two scFv wherein each sFv has a different specificity), or MSTAR-format antibody (for example, a monospecific or bispecific MSTAR) covalently linked to a sequence set forth in SEQ ID NOS: 473 to 511, wherein the antibody or antibodies (in the of a bispecific antigen-binding sequence) is/are selected from the group consisting of 3F8 (anti-GD2 ganglioside), abagovomab (anti-CA-125), abciximab (anti-CD41, integrin alpha-IIb), abituzumab (anti-CD51), abrezekimab (anti-IL-13), abrilumab (anti-integrin α4 β7), actoxumab (anti-Clostridium difficile), adalimumab (anti-TNF-α), adecatumumab (anti-EpCAM), aducanumab (anti-Amyloid beta), afasevikumab (anti-IL-17A, IL-17F), afelimomab (anti-TNF-α), alacizumab pegol[13] VEGFR2), alemtuzumab (anti-CD52), alirocumab (anti-PCSK9), altumomab pentetate (anti-carcinoembryonic antigen (CEA)), amatuximab[(anti-mesothelin), amivantamab (anti-epidermal growth factor receptor (EGFR), cMet), anatumomab mafenatox (anti-tumor-associated glycoprotein 72 (TAG-72)), andecaliximab (anti-gelatinase B), anetumab ravtansine (anti-mesothelin (MSLN)), anifrolumab (anti-IFN-α/β receptor), ansuvimab (anti-Ebola virus glycoprotein), anrukinzumab (anti-IL-13), apolizumab[(anti-HLA-DR), aprutumab ixadotin (anti-FGFR2), arcitumomab (anti-Carcinoembryonic antigen (CEA)), ascrinvacumab (anti-activin receptor-like kinase 1), aselizumab (anti-L-selectin (CD62L)), atezolizumab (anti-PD-L1), atidortoxumab (anti-Staphylococcus aureus alpha toxin), atinumab (anti-RTN4), atorolimumab (anti-Rhesus factor), avelumab (anti-PD-L1), azintuxizumab vedotin (anti-CD319), bamlanivimab (anti-spike protein receptor binding domain (RBD) of SARS-CoV-2), bapineuzumab (anti-β-amyloid), basiliximab (anti-CD25 (a chain of IL-2 receptor)), bavituximab (anti-phosphatidylserine), BCD-100 (anti-PD-1), bebtelovimab (anti-spike protein receptor binding domain (RBD) of SARS-CoV-2), bectumomab (anti-CD22), bedinvetmab (anti-nerve growth factor (NGF)), begelomab (anti-DPP4), belantamab mafodotin (anti-B-cell maturation antigen (BCMA)), belimumab (anti-B-cell activating factor (BAFF)), bemarituzumab (anti-FGFR2), benralizumab[(anti-CD125), berlimatoxumab (anti-Staphylococcus aureus bi-component leucocidin), bermekimab (anti-IL-1α), bersanlimab (anti-ICAM-1), bertilimumab (anti-CCL11 (eotaxin-1)), besilesomab (anti-carcinoembryonic antigen (CEA)-related antigen), bevacizumab (anti-VEGF-A), bezlotoxumab[(anti-Clostridium difficile), biciromab (anti-beta chain), bimagrumab (anti-ACVR2B), bimekizumab (anti-IL-17A, IL-17F, IL-17AF), birtamimab (anti-serum amyloid A protein), bivatuzumab (anti-CD44 v6), bleselumab (anti-CD40), blinatumomab (anti-CD19), blontuvetmab (anti-CD20), blosozumab (anti-SOST), bococizumab (anti-PCSK9), brazikumab (anti-IL-23), brentuximab vedotin (anti-CD30 (TNFRSF8)), briakinumab (anti-IL-12, IL-23), brodalumab (anti-IL-17), brolucizumab (anti-vascular endothelial growth factor A (VEGFA)), brontictuzumab (anti-Notch 1), burosumab (anti-FGF 23), cabiralizumab (anti-CSF1R), camidanlumab tesirine (anti-CD25 (a chain of IL-2 receptor), camrelizumab (anti-PD-1), canakinumab (anti-IL-1), cantuzumab mertansine (anti-CanAg (a glycoform of MUC1)), cantuzumab ravtansine (anti-CanAg (a glycoform of MUC1)), caplacizumab (anti-VWF), casirivimab (anti-spike protein receptor binding domain (RBD) of SARS-CoV-2), capromab (anti-Glutamate carboxypeptidase II), carlumab (anti-MCP-1), carotuximab (anti-endoglin), catumaxomab (anti-EpCAM, CD3), cBR96-doxorubicin immunoconjugate (anti-Lewis-Y antigen), cedelizumab (anti-CD4), cemiplimab (anti-PD-1), cergutuzumab amunaleukin (anti-IL-2), certolizumab pegol (anti-TNF-α), cetrelimab (anti-PD-1), cetuximab (anti-epidermal growth factor receptor (EGFR)), cibisatamab (anti-CEACAM5), cilgavimab (anti-spike protein receptor binding domain (RBD) of SARS-CoV-2), cirmtuzumab (anti-ROR1), citatuzumab bogatox (anti-EpCAM), cixutumumab (anti-IGF-1 receptor (CD221)), clazakizumab (anti-IL-6), clenoliximab (anti-CD4), clivatuzumab tetraxetan (anti-MUC1), codrituzumab (anti-glypican 3), cofetuzumab pelidotin (anti-PTK7), coltuximab ravtansine (anti-CD19), conatumumab (anti-TRAIL-R2), concizumab (anti-tissue factor pathway inhibitor (TFPI)), cosfroviximab (anti-ebolavirus glycoprotein), crenezumab (anti-β-amyloid (1-40 and 1-42)), crizanlizumab (anti-selectin P), crotedumab (anti-glucagon receptor (GCGR)), CR6261 (anti-Hemagglutinin (influenza)), cusatuzumab (anti-CD70), dacetuzumab (anti-CD40), daclizumab (anti-CD25 (a chain of IL-2 receptor), dalotuzumab (anti-IGF-1 receptor (CD221)), dapirolizumab pegol (anti-CD154 (CD40L)), daratumumab (anti-CD38), dectrekumab (anti-IL-13), demcizumab (anti-DLL4), denintuzumab mafodotin (anti-CD19), denosumab (anti-RANKL), depatuxizumab mafodotin (anti-EGFR), derlotuximab biotin (anti-histone complex), detumomab (anti-B-lymphoma cell), dezamizumab (anti-serum amyloid P component), dinutuximab (anti-GD2 ganglioside), dinutuximab beta (anti-GD2 ganglioside), diridavumab (anti-Hemagglutinin (influenza)), domagrozumab (anti-GDF-8), donanemab (anti-Amyloid beta), dostarlimab (anti-PCDP1), drozitumab (anti-DR5), DS-8201 (anti-HER2), duligotuzumab (anti-ERBB3 (HER3)), dupilumab (anti-IL-4Ra), durvalumab (anti-PD-L1), dusigitumab (anti-IGF-2), duvortuxizumab (anti-CD19, CD3E), ecromeximab (anti-GD3 ganglioside), eculizumab (anti-C5), edobacomab (anti-endotoxin), edrecolomab (anti-EpCAM), efalizumab (anti-LFA-1 (CD11a)), efungumab (anti-Hsp90), eldelumab (anti-CXCL10 (IP-10)), elezanumab (anti-repulsive guidance molecule A (RGMA)), elgemtumab (anti-ERBB3 (HER3)), elotuzumab (anti-SLAMF7), elsilimomab (anti-IL-6), emactuzumab (anti-CSF1R), emapalumab (anti-IFN-7), emibetuzumab (anti-HGFR), emicizumab (anti-activated F9, F10), enapotamab vedotin (anti-AXL), enavatuzumab (anti-TWEAK receptor), enfortumab vedotin (anti-nectin-4), enlimomab pegol (anti-ICAM-1 (CD54)), enoblituzumab (anti-CD276), enokizumab (anti-IL-9), enoticumab (anti-DLL4), ensituximab (anti-MUC5AC), epcoritamab (anti-CD3, CD20), epitumomab cituxetan (anti-episialin), epratuzumab (anti-CD22), eptinezumab (anti-calcitonin gene-related peptide), erenumab (anti-calcitonin gene-related peptide receptor (CGRP)), erlizumab (anti-ITGB2 (CD18)), ertumaxomab (anti-HER2/neu, CD3), etaracizumab (anti-integrin αvβ3), etesevimab (anti-spike protein receptor binding domain (RBD) of SARS-CoV-2), etigilimab (anti-TIGIT), etrolizumab (anti-integrin β7), evinacumab (anti-angiopoietin 3), evolocumab (anti-PCSK9), exbivirumab (anti-hepatitis B surface antigen), fanolesomab(anti-CD15), faralimomab (anti-IFN receptor), faricimab (anti-VEGF-A and Ang-2), farletuzumab (anti-folate receptor 1), fasinumab (anti-nerve growth factor (NGF)), FBTA05 (anti-CD20), felvizumab (anti-respiratory syncytial virus), fezakinumab (anti-IL-22), fibatuzumab (anti-ephrin receptor A3), ficlatuzumab (anti-Hepatocyte growth factor (HGF)), figitumumab (anti-IGF-1 receptor (CD221)), firivumab (anti-Hemagglutinin (influenza)), flanvotumab (anti-TYRP1 (glycoprotein 75)), fletikumab (anti-IL-20), flotetuzumab (anti-IL-3 receptor), fontolizumab (anti-IFN-7), foralumab (anti-CD3E), foravirumab (anti-rabies virus glycoprotein), fremanezumab (anti-calcitonin gene-related peptide alpha and beta), fresolimumab (anti-TGF-β), frovocimab (anti-PCSK9), frunevetmab (anti-nerve growth factor (NGF)), fulranumab (anti-nerve growth factor (NGF)), futuximab (anti-Epidermal growth factor receptor (EGFR)), galcanezumab (anti-calcitonin), galiximab (anti-CD80), gancotamab (anti-HER2/neu), ganitumab (anti-IGF-1 receptor (CD221)), gantenerumab (anti-β-amyloid (1-40 and 1-42)), gatipotuzumab (anti-MUC1), gavilimomab (anti-CD147 (basigin)), gedivumab (anti-Hemagglutinin (influenza)), gemtuzumab ozogamicin (anti-CD33), gevokizumab (anti-IL-10), gilvetmab (anti-PCDC1), gimsilumab (anti-CSF2), girentuximab (anti-carbonic anhydrase 9 (CA-IX)), glembatumumab vedotin (anti-GPNMB), glofitamab (anti-CD20, CD3), golimumab (anti-TNF-α), gomiliximab (anti-CD23 (IgE receptor), gosuranemab (anti-tau protein), guselkumab (anti-IL-23), ianalumab (anti-BAFF-R), ibalizumab (anti-CD4), sintilimab (anti-PD-1), ibritumomab tiuxetan (anti-CD20), icrucumab (anti-VEGFR-1), idarucizumab (anti-dabigatran), ifabotuzumab (anti-EPHA3), igovomab (anti-CA-125), iladatuzumab vedotin (anti-CD79B), imalumab (anti-macrophage migration inhibitory factor (MIF)), imaprelimab (anti-melanoma cell adhesion molecule (MCAM)), imciromab (anti-cardiac myosin), imdevimab (anti-spike protein receptor binding domain (RBD) of SARS-CoV-2), imgatuzumab (anti-Epidermal growth factor receptor (EGFR)), inclacumab (anti-selectin P), indatuximab ravtansine (anti-SDC1), indusatumab vedotin (anti-GUCY2C), inebilizumab (anti-CD19), infliximab (anti-TNF-α), intetumumab (anti-CD51), inolimomab (anti-CD25 (a chain of IL-2 receptor)), inotuzumab ozogamicin (anti-CD22), ipilimumab (anti-CD152), iomab-B (anti-CD45), iratumumab (anti-CD30 (TNFRSF8)), isatuximab (anti-CD38), iscalimab (anti-CD40), istiratumab (anti-IGF-1 receptor (CD221)), itolizumab (anti-CD6), ixekizumab (anti-IL-17A), keliximab (anti-CD4), labetuzumab (anti-Carcinoembryonic antigen (CEA)), lacnotuzumab (anti-CSF1, macrophage colony stimulating factor (MCSF)), ladiratuzumab vedotin (anti-LIV-1), lampalizumab (anti-Complement factor D (CFD)), lanadelumab (anti-kallikrein), landogrozumab (anti-GDF-8), laprituximab emtansine (anti-epidermal growth factor receptor (EGFR)), larcaviximab (anti-ebolavirus glycoprotein), lebrikizumab (anti-IL-13), lecanemab (anti-β-amyloid), lemalesomab (anti-NCA-90 (granulocyte antigen)), lendalizumab (anti-C5), lenvervimab (anti-hepatitis B surfage antigen), lenzilumab (anti-CSF2), lerdelimumab (anti-TGF-02), leronlimab (anti-CCR5), lesofavumab (anti-Hemagglutinin (influenza)), letolizumab (anti-tumor necrosis factor related activation protein (TRAP)), lexatumumab (anti-TRAIL-R2), libivirumab (anti-hepatitis B surface antigen), lifastuzumab vedotin (anti-phosphate-sodium co-transporter), ligelizumab (anti-IGHE), loncastuximab tesirine (anti-CD19), losatuxizumab vedotin (anti-EGRF, ERBB1 HER1), lilotomab satetraxetan (anti-CD37), lintuzumab (anti-CD33), lirilumab (anti-KIR2D), lodelcizumab (anti-PCSK9), lorvotuzumab mertansine (anti-CD56), lucatumumab (anti-CD40), lulizumab pegol (anti-CD28), lumiliximab (anti-CD23 (IgE receptor)), lumretuzumab (anti-ERBB3 (HER3)), lupartumab amadotin (anti-LYPD3), lutikizumab (anti-IL-1α), mapatumumab (anti-TRAIL-R1), margetuximab (anti-HER2), marstacimab (anti-tissue factor pathway inhibitor (TFPI)), maslimomab (anti-T cell receptor), mavrilimumab (anti-GMCSF receptor α-chain), matuzumab (anti-epidermal growth factor receptor (EGFR)), mepolizumab (anti-IL-5), metelimumab (anti-TGF-β1), milatuzumab (anti-CD74), minretumomab (anti-TAG-72), mirikizumab (anti-IL-23), mirvetuximab soravtansine (anti-folate receptor alpha), mitumomab (anti-GD3 ganglioside), modotuximab (anti-EGFR extracellular domain III), mogamulizumab (anti-CCR4), monalizumab (anti-NKG2A), morolimumab (anti-Rhesus factor), mosunetuzumab (anti-CD3E, MS4A1, CD20), motavizumab (anti-respiratory syncytial virus), moxetumomab pasudotox (anti-CD22), muromonab-CD3 (anti-CD3), nacolomab tafenatox (anti-C242 antigen), namilumab (anti-CSF2), naptumomab estafenatox (anti-5T4)), naratuximab emtansine (anti-CD37)), narnatumab (anti-MST1R (aka RON)), natalizumab (anti-integrin α4), navicixizumab (anti-DLL4 and VEGFA), navivumab (anti-Hemagglutinin (influenza)), naxitamab (anti-c-Met), nebacumab (anti-endotoxin), necitumumab (anti-epidermal growth factor receptor (EGFR)), nemolizumab (anti-IL-31 receptor A), NEOD001 (anti-amyloid), nerelimomab (anti-TNF-α, nesvacumab (anti-angiopoietin 2), netakimab (anti-IL-17A), nimotuzumab (anti-epidermal growth factor receptor (EGFR)), nirsevimab (anti-RSV fusion glycoprotein), nivolumab (anti-PD-1), nofetumomab merpentan (pancarcinoma murine antibody NR-LU-10 linked with gamma-emitting radioisotope technetium 99m (Tc 99m)), obiltoxaximab (anti-Bacillus anthracis anthrax), obinutuzumab anti-CD20, ocaratuzumab (anti-CD20), ocrelizumab (anti-CD20). atoltivimab/maftivimab/odesivimab (INMAZEB®, REGN-EB3) (anti-Zaire ebolavirus glycoprotein combination therapy), odulimomab (anti-LFA-1 (CD11a)), ofatumumab (anti-CD20), olaratumab (anti-PDGFRA), oleclumab (anti-5′-nucleotidase), olendalizumab (anti-complement C5a), olokizumab (anti-IL-6), omalizumab (anti-IgE Fc region), omburtamab (anti-CD276), oMS721 (anti-MASP-2), onartuzumab (anti-human scatter factor receptor kinase), ontuxizumab (anti-TEM1), onvatilimab (anti-VISTA (protein) (VSIR)); opicinumab (anti-LINGO-1), oportuzumab monatox (anti-EpCAM), oregovomab (anti-CA-125), orticumab (anti-oxLDL), otelixizumab (anti-CD3), otilimab (anti-GMCSF), otlertuzumab (anti-CD37), oxelumab (anti-OX-40), ozanezumab (anti-NOGO-A), ozoralizumab (anti-TNF-α), pagibaximab (anti-lipoteichoic acid), palivizumab (anti-F protein of respiratory syncytial virus), pamrevlumab (anti-connective tissue growth factor (CTGF)), panitumumab (anti-epidermal growth factor receptor (EGFR)), pankomab (anti-tumor specific glycosylation of MUC1), panobacumab (anti-Pseudomonas aeruginosa), parsatuzumab (anti-EGFL7), pascolizumab (anti-IL-4), pasotuxizumab (anti-folate hydrolase), pateclizumab (anti-lymphotoxin alpha (LTA)), patritumab (anti-ERBB3 (HER3)), PDR001 (anti-PD-1), pembrolizumab (anti-PD-1), pemtumomab (anti-MUC1), perakizumab (anti-IL-17A), pertuzumab (anti-HER2/neu), pexelizumab (anti-C5), pidilizumab (anti-PD-1), pinatuzumab vedotin (anti-CD22), pintumomab (anti-adenocarcinoma antigen), placulumab (anti-TNF), pozelimab (anti-C5), prezalumab (anti-TNF), plozalizumab (anti-CCR2), pogalizumab (anti-tumor necrosis factor receptor (TNFR) superfamily member 4), polatuzumab vedotin (anti-CD79B), ponezumab (anti-β-amyloid), porgaviximab (anti-Zaire ebolavirus glycoprotein), prasinezumab (anti-Alpha-synuclein), prezalizumab (anti-inducible T cell co-stimulatory ligand (ICOSL)), priliximab (anti-CD4), pritoxaximab (anti-E. coli shiga toxin type-1), pritumumab (anti-vimentin), PRO 140 (anti-CCR5), quilizumab (anti-IGHE), racotumomab (anti-NGNA ganglioside), radretumab (anti-fibronectin extra domain-B), rafivirumab (anti-rabies virus glycoprotein), ralpancizumab (anti-PCSK9), ramucirumab (anti-VEGFR2), ranevetmab (anti-NGF), ranibizumab (anti-VEGF-A), raxibacumab (anti-anthrax toxin protective antigen), ravagalimab (anti-CD40), ravulizumab (anti-C5), refanezumab (anti-myelin-associated glycoprotein), regavirumab (anti-cytomegalovirus glycoprotein B), regdanvimab (anti-spike protein receptor binding domain (RBD) of SARS-CoV-2), relatlimab (anti-LAG3), remtolumab (anti-IL-17A, TNF), reslizumab (anti-IL-5), retifanlimab (anti-PD-1), rilotumumab (anti-hepatocyte growth factor (HGF)), rinucumab (anti-PDGFRB), risankizumab (anti-IL-23A), rituximab (anti-CD20), rivabazumab pegol (anti-Pseudomonas aeruginosa type III secretion system), robatumumab (anti-IGF-1 receptor (CD221)), rmab (anti-rabies virus G glycoprotein), roledumab (anti-RHD (gene) (RHD)), romilkimab (anti-IL-13), romosozumab (anti-sclerostin), rontalizumab (anti-IFN-α), rosmantuzumab (anti-root plate-specific spondin 3), rovalpituzumab tesirine (anti-DLL3), rovelizumab (anti-CD11, anti-CD18), rozanolixizumab (anti-FCGRT), ruplizumab (anti-CD154 (CD40L)), SA237 (anti-IL-6 receptor), sacituzumab govitecan (anti-TROP-2), samalizumab (anti-CD200), samrotamab vedotin (anti-LRRC15), sarilumab (anti-IL-6), satralizumab (anti-IL-6 receptor), satumomab pendetide (anti-TAG-72), secukinumab (anti-IL-17A), selicrelumab (anti-CD40), seribantumab (anti-ERBB3 (HER3)), setoxaximab (anti-E. coli shiga toxin type-2), setrusumab (anti-sclerostin (SOST)), sevirumab (anti-cytomegalovirus), sibrotuzumab (anti-FAP (gene) (FAP)), SGN-CD19A (anti-CD19), SHP647 (anti-mucosal addressin cell adhesion molecule), sifalimumab (anti-IFN-α), siltuximab (anti-IL-6), simtuzumab (anti-LOXL2), siplizumab (anti-CD2), sirtratumab vedotin (anti-SLITRK6), sirukumab (anti-IL-6), sofituzumab vedotin (anti-CA-125), solanezumab (anti-β-amyloid), solitomab (anti-EpCAM), sonepcizumab (anti-sphingosine-1-phosphate), sontuzumab (anti-episialin), sotrovimab (anti-spike protein receptor binding domain (RBD) of SARS-CoV-2), spartalizumab (anti-PD-1), spesolimab (anti-Interleukin 36 receptor (IL1RL2/IL1RAP)), stamulumab (anti-myostatin), sulesomab (anti-NCA-90 (granulocyte antigen)), suptavumab (anti-RSVFR), sutimlimab (anti-complement component is (CIs)), suvizumab (anti-HIV-1), suvratoxumab (anti-Staphylococcus aureus alpha toxin), tabalumab (anti-B-cell activating factor (BAFF)), tacatuzumab tetraxetan (anti-alpha-fetoprotein), tadocizumab (anti-integrin αIIbβ3), tafasitamab (anti-CD19), talacotuzumab (anti-CD123), talizumab (anti-IgE), talquetamab (anti-GPRC5D, anti-CD3), tamtuvetmab (anti-CD52), tanezumab (anti-nerve growth factor (NGF)), taplitumomab paptox (anti-CD19), tarextumab (anti-Notch receptor), tavolimab (anti-CD134), teclistamab (anti-B-cell maturation antigen (BCMA), CD3), tefibazumab (anti-clumping factor A), telimomab aritox (anti-CD5), telisotuzumab (anti-HGFR), telisotuzumab vedotin (anti-HGFR), tenatumomab (anti-tenascin C), teneliximab (anti-CD40), teplizumab (anti-CD3), tepoditamab (anti-dendritic cell-associated lectin 2), teprotumumab (anti-IGF-1 receptor (CD221)), tesidolumab (anti-C5), tetulomab (anti-CD37), tezepelumab (anti-thymic stromal lymphopoietin (TSLP)), TGN1412 (anti-CD28), tibulizumab (anti-B-cell activating factor (BAFF)), tildrakizumab (anti-IL-23), tigatuzumab (anti-TRAIL-R2), timigutuzumab (anti-HER2), timolumab (anti-AOC3), tiragotumab (anti-TIGIT), tislelizumab (anti-PCDC1, anti-CD279), tisotumab vedotin (anti-coagulation factor III), tixagevimab (anti-spike protein receptor binding domain (RBD) of SARS-CoV-2), TNX-650 (anti-IL-13), tocilizumab (anti-IL-6 receptor), tomuzotuximab (anti-Epidermal growth factor receptor (EGFR), anti-HER1), toralizumab (anti-CD154 (CD40L)), tosatoxumab (anti-Staphylococcus aureus), tositumomab (anti-CD20), tovetumab (anti-PDGFRA), tralokinumab (anti-IL-13), trastuzumab (anti-HER2/neu), trastuzumab duocarmazine (anti-HER2/neu), trastuzumab emtansine (anti-HER2/neu), TRBS07 (anti-GD2 ganglioside), tregalizumab (anti-CD4), tremelimumab (anti-CTLA-4), trevogrumab (anti-growth differentiation factor 8), tucotuzumab celmoleukin (anti-EpCAM), tuvirumab (anti-hepatitis B virus), ublituximab (anti-CD20), ulocuplumab (anti-CXCR4 (CD184)), urelumab (anti-4-1BB (CD137)), urtoxazumab (anti-Escherichia coli), ustekinumab (anti-IL-12, anti-IL-23), utomilumab (anti-4-1BB (CD137)), vadastuximab talirine (anti-CD33), vanalimab (anti-CD40), vandortuzumab vedotin (anti-STEAP1), vantictumab (anti-Frizzled receptor), vanucizumab (anti-angiopoietin 2), vapaliximab (anti-AOC3/VAP-1), varisacumab (anti-VEGF-A), varlilumab (anti-CD27), vatelizumab (anti-ITGA2/CD49b), vedolizumab (anti-integrin α4 β7), veltuzumab (anti-CD20), vepalimomab (anti-AOC3, a.k.a., VAP-1), vesencumab (anti-NRP1), vilobelimab (anti-C5a receptor; C5a), visilizumab (anti-CD3), vobarilizumab (anti-IL-6 receptor), volociximab (anti-integrin α5β1), vonlerolizumab (anti-CD134), vopratelimab (anti-CD278, a.k.a. ICOS), vorsetuzumab mafodotin (anti-CD70), votumumab (anti-tumor antigen CTAA16.88), vunakizumab (anti-IL-17A), xentuzumab (anti-IGF-1, anti-IGF-2), XMAB-5574 (anti-CD19), zalutumumab (anti-Epidermal growth factor receptor, EGFR), zanolimumab (anti-CD4), zatuximab (an anti-HER1), zenocutuzumab (anti-ERBB3/HER3), ziralimumab (anti-CD147/basigin), zolbetuximab (anti-claudin 18 isoform 2), and zolimomab aritox (anti-CD5).

In some aspects, the CAR comprises an antigen-binding domain covalently attached to a sequence set forth in SEQ ID NOS: 473 to 511.

In some aspects, the CAR has a polypeptide structure represented by
[Antigen Binding Domain]-[Optional Spacer]-[Spacer/Transmembrane/Intracellular Domain], or in abbreviated form

[ABD]-[OS]—[STID]
wherein
[Antigen Binding Domain] or [ABD] is the extracellular antigen recognition domain of the CAR and comprises an antibody or antigen binding fragment thereof, for example, an MSTAR antibody;
[Optional Spacer] or [OS] is an optional spacer that comprises, e.g., an antibody hinge region or fragment thereof, or a Gly-Ser spacer; and,
[Spacer/Transmembrane/Intracellular Domain] or [STID] comprises, consists, or consists essentially of a sequence set forth in SEQ ID NOS: 473 to 511, or a sequence derived from a sequence set forth in SEQ ID NOS: 473 to 511, minus 1, 2, 3, 4, 5, 6, 7, 8, 9, 10, 11, 12, 13, 14, 15, 16, 17, 18, 19 OR 20N-terminal amino acids.

In some aspects, [STID] is a functional variant (protein with one or more mutations/substitutions with respect to a parent protein, having the functional characteristics as the parent protein) which about 85%, about 90%, about 95%, about 96%, about 97%, about 98%, or about 99% sequence identity to a sequence set forth in any one of SEQ ID NOS: 473 to 511.

In some aspects, [STID] consists of the transmembrane and intracellular portions, subsequences, or domains of a CAR disclosed in TABLE 1.

In some aspects, [STID] corresponds to domains D3-D5 of SEQ ID NO: 300 (TRANSMEMBRANE-REGION (243-311) [D3]+TNFRSF9 (Pr214-255) (312-353) [D4]+CD247 (Pr52-164) (354-465) [D5]). Thus, in some aspects, a CAR of the present disclosure comprises a MSTAR antigen-binding domain covalently attached to domains D3-D5 (positions 243 to 465) of SEQ ID NO: 300, either directly or via a linker or spacer disclosed herein.

In some aspects, [STID] corresponds to domains D3-D4 of SEQ ID NO: 301 (CD28 (Pr114-220) (249-355) [D3]+CD247 (Pr52-164) (356-467) [D4]). Thus, in some aspects, a CAR of the present disclosure comprises a MSTAR antigen-binding domain covalently attached to domains D3-D4 (positions 249 to 467) of SEQ ID NO: 301, directly or via a linker or spacer disclosed herein.

In some aspects, [STID] corresponds to domains D3-D5 of SEQ ID NO: 302 (TRANSMEMBRANE-REGION (243-311) [D3]+TNFRSF9 (Pr214-255) (334-353) [D4]+CD247 (Pr52-164) (354-465) [D5]). Thus, in some aspects, a CAR of the present disclosure comprises a MSTAR antigen-binding domain covalently attached to domains D3-D5 (positions 243 to 465) of SEQ ID NO: 302, either directly or via a linker or spacer disclosed herein.

In some aspects, [STID] corresponds to domains D3-D4 of SEQ ID NO: 303 (CD28 (Pr114-220) (249-355) [D3]+CD247 (Pr52-164) (356-467) [D4]). Thus, in some aspects, a CAR of the present disclosure comprises a MSTAR antigen-binding domain covalently attached to domains D3-D4 (positions 249 to 467) of SEQ ID NO: 303, directly or via a linker or spacer disclosed herein.

In some aspects, [STID] corresponds to domains D3-D5 of SEQ ID NO: 304 (TRANSMEMBRANE-REGION (252-321) [D3]+TNFRSF9 (Pr214-255) (322-363) [D4]+CD247 (Pr52-164) (364-475) [D5]). Thus, in some aspects, a CAR of the present disclosure comprises a MSTAR antigen-binding domain covalently attached to domains D3-D5 (positions 252 to 475) of SEQ ID NO: 304, either directly or via a linker or spacer disclosed herein.

In some aspects, [STID] corresponds to domains D3-D5 of SEQ ID NO: 305 (CD8A (257-329) [D3]+CD28 (Pr114-220) (332-372) [D4]+CD247 (Pr52-164) (373-484) [D5]). Thus, in some aspects, a CAR of the present disclosure comprises a MSTAR antigen-binding domain covalently attached to domains D3-D5 (positions 257 to 484) of SEQ ID NO: 305, either directly or via a linker or spacer disclosed herein.

In some aspects, [STID] corresponds to domains D3-D5 of SEQ ID NO: 306 (TRANSMEMBRANE-REGION (248-316) [D3]+TNFRSF9 (Pr214-255) (317-358) [D4]+CD247 (Pr52-164) (359-470) [D5]). Thus, in some aspects, a CAR of the present disclosure comprises a MSTAR antigen-binding domain covalently attached to domains D3-D5 (positions 316 to 470) of SEQ ID NO: 306, either directly or via a linker or spacer disclosed herein.

In some aspects, [STID] corresponds to domains D3-D6 of SEQ ID NO: 307 (CD28 (Pr153-179) (259-285) [D3]+TNFRSF9 (Pr214-255) (286-327) [D4]+CD247 (Pr52-164) (328-439) [D5]+EGFR (Pr28-459) (486-657) [D6]). Thus, in some aspects, a CAR of the present disclosure comprises a MSTAR antigen-binding domain covalently attached to domains D3-D6 (positions 259 to 657) of SEQ ID NO: 307, either directly or via a linker or spacer disclosed herein.

In some aspects, [STID] corresponds to domains D3-D5 of SEQ ID NO: 308 (TRANSMEMBRANE-REGION (244-313) [D3]+TNFRSF9 (Pr214-255) (314-355) [D4]+CD247 (Pr52-164) (356-406) [D5]). Thus, in some aspects, a CAR of the present disclosure comprises a MSTAR antigen-binding domain covalently attached to domains D3-D5 (positions 244 to 406) of SEQ ID NO: 308, either directly or via a linker or spacer disclosed herein.

In some aspects, [STID] corresponds to domains D2-D4 of SEQ ID NO: 309 (TRANSMEMBRANE-REGION (84-152) [D2]+TNFRSF9 (Pr214-255) (153-194) [D3]+CD247 (Pr52-164) (195-306) [D4]). Thus, in some aspects, a CAR of the present disclosure comprises a MSTAR antigen-binding domain covalently attached to domains D2-D4 (positions 84 to 306) of SEQ ID NO: 309, either directly or via a linker or spacer disclosed herein.

In some aspects, [STID] corresponds to domains D3-D5 of SEQ ID NO: 310 (TRANSMEMBRANE-REGION (245-324) [D3]+TNFRSF9 (Pr214-255) (325-355) [D4]+CD247 (Pr52-164) (356-467) [D5]). Thus, in some aspects, a CAR of the present disclosure comprises a MSTAR antigen-binding domain covalently attached to domains D3-D5 (positions 245 to 467) of SEQ ID NO: 310, either directly or via a linker or spacer disclosed herein.

In some aspects, [STID] corresponds to domains D3-D5 of SEQ ID NO: 312 (TRANSMEMBRANE-REGION (250-318) [D3]+TNFRSF9 (Pr214-255) (319-360) [D4]+CD247 (Pr52-164) (361-472) [D5]). Thus, in some aspects, a CAR of the present disclosure comprises a MSTAR antigen-binding domain covalently attached to domains D3-D5 (positions 250 to 472) of SEQ ID NO: 312, either directly or via a linker or spacer disclosed herein.

In some aspects, [STID] corresponds to domains D3-D5 of SEQ ID NO: 313 (TRANSMEMBRANE-REGION (252-324) [D3]+TNFRSF9 (Pr214-255) (327-368) [D4]+CD247 (Pr52-164) (369-480) [D5]). Thus, in some aspects, a CAR of the present disclosure comprises a MSTAR antigen-binding domain covalently attached to domains D3-D5 (positions 252 to 480) of SEQ ID NO: 313, either directly or via a linker or spacer disclosed herein.

In some aspects, [STID] corresponds to domains D4-D5 of SEQ ID NO: 314 (C-LIKE (418-431) [D4]+CYTOPLASMIC-REGION (432-501) [D5]). Thus, in some aspects, a CAR of the present disclosure comprises a MSTAR antigen-binding domain covalently attached to domains D4-D5 (positions 418 to 501) of SEQ ID NO: 314, directly or via a linker or spacer disclosed herein.

In some aspects, [STID] corresponds to domains D3-D5 of SEQ ID NO: 315 (TRANSMEMBRANE-REGION (240-308) [D3]+TNFRSF9 (Pr214-255) (309-350) [D4]+CD247 (Pr52-164) (351-462) [D5]). Thus, in some aspects, a CAR of the present disclosure comprises a MSTAR antigen-binding domain covalently attached to domains D3-D5 (positions 240 to 462) of SEQ ID NO: 315, either directly or via a linker or spacer disclosed herein.

In some aspects, [STID] corresponds to domains D2-D5 of SEQ ID NO: 316 (CD3E C-LIKE (132-226) [D2]+CD3E CO (227-241) [D3]+CD3E TM (242-257) [D4]+CD3E CY (258-316) [D5]). Thus, in some aspects, a CAR of the present disclosure comprises a MSTAR antigen-binding domain covalently attached to domains D2-D5 (positions 132 to 316) of SEQ ID NO: 316, either directly or via a linker or spacer disclosed herein.

In some aspects, [STID] corresponds to domains D3-D5 of SEQ ID NO: 317 (CD8A (250-318) [D3]+TNFRSF9 (Pr214-255) (319-360) [D4]+CD247 (Pr52-164) (361-472) [D5]). Thus, in some aspects, a CAR of the present disclosure comprises a MSTAR antigen-binding domain covalently attached to domains D3-D5 (positions 250 to 472) of SEQ ID NO: 317, either directly or via a linker or spacer disclosed herein.

In some aspects, [STID] corresponds to domains D5-D6 of SEQ ID NO: 318 (CD28 (Pr114-220) (487-554) [D5]+CD247 (Pr52-164) (555-666) [D6]). Thus, in some aspects, a CAR of the present disclosure comprises a MSTAR antigen-binding domain covalently attached to domains D5-D6 (positions 487 to 666) of SEQ ID NO: 318, directly or via a linker or spacer disclosed herein.

In some aspects, [STID] corresponds to domains D3-D5 of SEQ ID NO: 319 (TRANSMEMBRANE-REGION (258-330) [D3]+TNFRSF9 (Pr214-255) (334-375) [D4]+CD247 (Pr52-164) (376-487) [D5]). Thus, in some aspects, a CAR of the present disclosure comprises a MSTAR antigen-binding domain covalently attached to domains D3-D5 (positions 258 to 487) of SEQ ID NO: 319, either directly or via a linker or spacer disclosed herein.

In some aspects, [STID] corresponds to domains D3-D5 of SEQ ID NO: 320 (TRANSMEMBRANE-REGION (244-313) [D3]+TNFRSF9 (Pr214-255) (314-355) [D4]+CD247 (Pr52-164) (356-406) [D5]). Thus, in some aspects, a CAR of the present disclosure comprises a MSTAR antigen-binding domain covalently attached to domains D3-D5 (positions 244 to 406) of SEQ ID NO: 320, either directly or via a linker or spacer disclosed herein.

In some aspects, [STID] corresponds to domains D5-D8 of SEQ ID NO: 321 (CD28 (Pr114-220) (472-509) [D5]+TNFRSF9 (Pr214-255) (510-542) [D6]+CD247 (Pr52-164) (543-654) [D7]+EGFR1 (701-1035) [D8]). Thus, in some aspects, a CAR of the present disclosure comprises a MSTAR antigen-binding domain covalently attached to domains D5-D8 (positions 472 to 1035) of SEQ ID NO: 321, either directly or via a linker or spacer disclosed herein.

In some aspects, [STID] corresponds to domains D3-D6 of SEQ ID NO: 322 (CD3E C-LIKE (263-357) [D3]+CD3E CO (358-372) [D4]+CD3E TM (373-388) [D5]+CD3E CY (389-447) [D6]). Thus, in some aspects, a CAR of the present disclosure comprises a MSTAR antigen-binding domain covalently attached to domains D3-D6 (positions 263 to 447) of SEQ ID NO: 322, either directly or via a linker or spacer disclosed herein.

In some aspects, [STID] corresponds to domains D3-D6 of SEQ ID NO: 323 (TRANSMEMBRANE-REGION (253-328) [D3]+CD28 (Pr114-220) (329-368) [D4]+TNFRSF9 (Pr214-255) (374-415) [D5]+CD247 (Pr52-164) (416-527) [D6]). Thus, in some aspects, a CAR of the present disclosure comprises a MSTAR antigen-binding domain covalently attached to domains D3-D6 (positions 253 to 527) of SEQ ID NO: 323, directly or via a linker or spacer disclosed herein.

In some aspects, [STID] corresponds to domains D5-D7 of SEQ ID NO: 324 (CD28 (Pr153-179) (511-537) [D5]+TNFRSF9 (Pr214-255) (538-579) [D6]+CD247 (Pr52-164) (580-691) [D7]). Thus, in some aspects, a CAR of the present disclosure comprises a MSTAR antigen-binding domain covalently attached to domains D5-D7 (positions 511 to 691) of SEQ ID NO: 324, either directly or via a linker or spacer disclosed herein.

In some aspects, [STID] corresponds to domains D3-D5 of SEQ ID NO: 325 (TRANSMEMBRANE-REGION (243-311) [D3]+TNFRSF9 (Pr214-255) (312-353) [D4]+CD247 (Pr52-164) (354-465) [D5]). Thus, in some aspects, a CAR of the present disclosure comprises a MSTAR antigen-binding domain covalently attached to domains D3-D5 (positions 243 to 465) of SEQ ID NO: 325, either directly or via a linker or spacer disclosed herein.

In some aspects, [STID] corresponds to domains D3-D6 of SEQ ID NO: 326 (CD28 (Pr153-179) (259-285) [D3]+TNFRSF9 (Pr214-255) (286-327) [D4]+CD247 (Pr52-164) (328-439) [D5]+EGFR1 (486-820) [D6]). Thus, in some aspects, a CAR of the present disclosure comprises a MSTAR antigen-binding domain covalently attached to domains D3-D6 (positions 259 to 820) of SEQ ID NO: 326, either directly or via a linker or spacer disclosed herein.

In some aspects, [STID] corresponds to domains D2-D4 of SEQ ID NO: 327 (TRANSMEMBRANE-REGION (123-193) [D2]+TNFRSF9 (Pr214-255) (194-235) [D3]+CD247 (Pr52-164) (236-348) [D4]). Thus, in some aspects, a CAR of the present disclosure comprises a MSTAR antigen-binding domain covalently attached to domains D2-D4 (positions 123 to 348) of SEQ ID NO: 327, either directly or via a linker or spacer disclosed herein.

In some aspects, [STID] corresponds to domains D3-D5 of SEQ ID NO: 328 (TRANSMEMBRANE-REGION (248-292) [D3]+CD28 (Pr114-220) (293-360) [D4]+CD247 (Pr52-164) (361-473) [D5]). Thus, in some aspects, a CAR of the present disclosure comprises a MSTAR antigen-binding domain covalently attached to domains D3-D5 (positions 248 to 473) of SEQ ID NO: 328, either directly or via a linker or spacer disclosed herein.

In some aspects, [STID] corresponds to domains D3-D4 of SEQ ID NO: 329 (CD28 (Pr114-220) (249-355) [D3]+CD247 (Pr52-164) (356-467) [D4]). Thus, in some aspects, a CAR of the present disclosure comprises a MSTAR antigen-binding domain covalently attached to domains D3-D4 (positions 249 to 467) of SEQ ID NO: 329, directly or via a linker or spacer disclosed herein.

In some aspects, [STID] corresponds to domains D5-D7 of SEQ ID NO: 330 (TRANSMEMBRANE-REGION (496-568) [D5]+TNFRSF9 (Pr214-255) (571-612) [D6]+CD247 (Pr52-164) (613-724) [D7]). Thus, in some aspects, a CAR of the present disclosure comprises a MSTAR antigen-binding domain covalently attached to domains D5-D7 (positions 496 to 724) of SEQ ID NO: 330, either directly or via a linker or spacer disclosed herein.

In some aspects, [STID] corresponds to domains D3-D5 of SEQ ID NO: 331 (TRANSMEMBRANE-REGION (252-322) [D3]+TNFRSF9 (Pr214-255) (323-364) [D4]+CD247 (Pr52-164) (365-476) [D5]). Thus, in some aspects, a CAR of the present disclosure comprises a MSTAR antigen-binding domain covalently attached to domains D3-D5 (positions 252 to 476) of SEQ ID NO: 331, either directly or via a linker or spacer disclosed herein.

In some aspects, [STID] corresponds to domains D3-D4 of SEQ ID NO: 332 (CD28 (Pr114-220) (249-355) [D3]+CD247 (Pr52-164) (356-467) [D4]). Thus, in some aspects, a CAR of the present disclosure comprises a MSTAR antigen-binding domain covalently attached to domains D3-D4 (positions 249 to 467) of SEQ ID NO: 332, directly or via a linker or spacer disclosed herein.

In some aspects, [STID] corresponds to domains D3-D5 of SEQ ID NO: 333 (TRANSMEMBRANE-REGION (256-324) [D3]+TNFRSF9 (Pr214-255) (325-366) [D4]+CD247 (Pr52-164) (367-479) [D5]). Thus, in some aspects, a CAR of the present disclosure comprises a MSTAR antigen-binding domain covalently attached to domains D3-D5 (positions 256 to 479) of SEQ ID NO: 333, either directly or via a linker or spacer disclosed herein.

In some aspects, [STID] corresponds to domains D3-D5 of SEQ ID NO: 334 (TRANSMEMBRANE-REGION (258-330) [D3]+TNFRSF9 (Pr214-255) (334-375) [D4]+CD247 (Pr52-164) (376-487) [D5]). Thus, in some aspects, a CAR of the present disclosure comprises a MSTAR antigen-binding domain covalently attached to domains D3-D5 (positions 258 to 487) of SEQ ID NO: 334, either directly or via a linker or spacer disclosed herein.

In some aspects, [STID] corresponds to domains D3-D5 of SEQ ID NO: 336 (TRANSMEMBRANE-REGION (245-313) [D3]+TNFRSF9 (Pr214-255) (314-355) [D4]+CD247 (Pr52-164) (356-468) [D5]). Thus, in some aspects, a CAR of the present disclosure comprises a MSTAR antigen-binding domain covalently attached to domains D3-D5 (positions 245 to 468) of SEQ ID NO: 336, either directly or via a linker or spacer disclosed herein.

In some aspects, [STID] corresponds to domains D3-D5 of SEQ ID NO: 344 (CD8A (243-311) [D3]+TNFRSF9 (Pr214-255) (312-353) [D4]+CD247 (Pr52-164) (354-465) [D5]). Thus, in some aspects, a CAR of the present disclosure comprises a MSTAR antigen-binding domain covalently attached to domains D3-D5 (positions 243-465) of SEQ ID NO: 344, either directly or via a linker or spacer disclosed herein.

In some aspects, the CAR has a polypeptide structure represented by [ABD]-[OS]—[STID] wherein [ABD] has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; and L1, L2 and L3 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] comprises a first polypeptide having a structure represented by VL1-VL2-VH2-VH1; VH1-VH2-VL2-VL1; VL1-L1-VL2-L2-VH2-L3-VH1; or VH1-L1-VH2-L2-VL2-L3-VL1; and a second polypeptide having a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; and L1, L2 and L3 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH2), an immunoglobulin heavy chain constant region 3 (CH3), and optionally, an immunoglobulin hinge; and L1, L2, L3 and L4 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] comprises a first polypeptide having a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; and a second polypeptide having a structure represented by Fc; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc VL1-L1-VL2-L2-VH2-L3-VH1-L4-Fc; or VH1-L1-VH2-L2-VL2-L3-VL1-L4-Fc; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH2), an immunoglobulin heavy chain constant region 3 (CH3), and optionally, an immunoglobulin hinge; and L1, L2, L3 and L4 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] has a structure represented by VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; and L1, L2, L3, L4 and L5 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] comprises a first polypeptide having a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; and a second polypeptide having a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; and L1, L2, L3, L4 and L5 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] has a structure represented by VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] comprises a first polypeptide having a structure represented by VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH-L1-VH2-L2-VL2-L3-VL-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc;
and a second polypeptide having a structure represented by Fc; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] comprises a first polypeptide having a structure represented by VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fe; VL1-L1-VL2-L2-VH2-L3-VH1-L4-Fe; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; and a second polypeptide having a structure represented by Fc; VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fe; VL1-L1-VL2-L2-VH2-L3-VH1-L4-CL-L5-CH1-Fe; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] has a structure represented by VL1-VL2-VH2-VH1-Fc-Fc; VH1-VH2-VL2-VL1-Fc-Fc; VL1-L1-VL2-L2-VH2-L3-VH1-Fc-Fc; VH1-L1-VH2-L2-VL2-L3-VL1-Fc-Fc; VL1-L1-VL2-L2-VH2-L3-VH1-L4-Fc-Fc; VH1-L1-VH2-L2-VL2-L3-VL1-L4-Fc-Fc; VL1-L1-VL2-L2-VH2-L3-VH1-L4-Fc-L5-Fc; or VH1-L1-VH2-L2-VL2-L3-VL1-L4-Fc-L5-Fc; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH2), an immunoglobulin heavy chain constant region 3 (CH3), and optionally, an immunoglobulin hinge; and L1, L2, L3, L4 and L5 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

In some aspects, the CAR has a polypeptide structure represented by [ABD]-[OS]—[STID] wherein [ABD] has a structure represented by VL1-VL2-VH2-VH1-CH3; VH1-VH2-VL2-VL1-CH3; VL1-L1-VL2-L2-VH2-L3-VH1-CH3; VH1-L1-VH2-L2-VL2-L3-VL1-CH3; VL1-L1-VL2-L2-VH2-L3-VH1-L4-CH3; VH1-L1-VH2-L2-VL2-L3-VL1-L4-CH3; VL1-VL2-VH2-VH1-CH3-CH3; VH1-VH2-VL2-VL1-CH3-CH3; VL1-L1-VL2-L2-VH2-L3-VH1-CH3-CH3; VH1-L1-VH2-L2-VL2-L3-VL1-CH3-CH3; VL1-L1-VL2-L2-VH2-L3-VH1-L4-CH3-CH3; VH1-L1-VH2-L2-VL2-L3-VL1-L4-CH3-CH3; VL1-L1-VL2-L2-VH2-L3-VH1-L4-CH3-L5-CH3; or VH1-L1-VH2-L2-VL2-L3-VL1-L4-CH3-L5-CH3; wherein VL1 is a first immunoglobulin light chain variable region that specifically binds to a first T cell specific surface protein; VL2 is a second immunoglobulin light chain variable region that specifically binds to a second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH3 is an immunoglobulin heavy chain constant region 3; and L1, L2, L3, L4 and L5 are amino acid linkers; wherein [OS] is an optional spacer that comprises, e.g., an antibody hinge region or fragment thereof, or a Gly-Ser spacer; and [STID] comprises a sequence set forth in SEQ ID NOS: 473 to 511.

Anti-CD19 and/or Anti-CD20 antigen recognition domains: In some aspects, the CAR of the present disclosure comprises an extracellular antigen recognition domain comprising a VH and/or VL from an anti-CD19 or anti-CD20 disclosed in TABLE 2.

In some aspects, the extracellular antigen recognition domain comprises a VH from an anti-CD19 disclosed in TABLE 2. In some aspects, the extracellular antigen recognition domain comprises a VL from an anti-CD19 disclosed in TABLE 2. In some aspects, the extracellular antigen recognition domain comprises a VL and a VH from an anti-CD19 disclosed in TABLE 2.

In some aspects, the extracellular antigen recognition domain comprises a VH from an anti-CD20 disclosed in TABLE 2. In some aspects, the extracellular antigen recognition domain comprises a VL from an anti-CD20 disclosed in TABLE 2. In some aspects, the extracellular antigen recognition domain comprises a VL and a VH from an anti-CD20 disclosed in TABLE 2.

In some aspects, the extracellular antigen recognition domain comprises the three VH CDRs from an anti-CD19 disclosed in TABLE 2. In some aspects, the extracellular antigen recognition domain comprises the three VL CDRs from an anti-CD19 disclosed in TABLE 2. In some aspects, the extracellular antigen recognition domain comprises the three VL CDRs and the three VH CDRs from an anti-CD19 disclosed in TABLE 2.

In some aspects, the extracellular antigen recognition domain comprises the three VH CDRs from an anti-CD20 disclosed in TABLE 2. In some aspects, the extracellular antigen recognition domain comprises the three VL CDRs from an anti-CD20 disclosed in TABLE 2. In some aspects, the extracellular antigen recognition domain comprises the three VL CDRs and the three VH CDRs from an anti-CD20 disclosed in TABLE 2.

In some aspects, the extracellular antigen recognition domain comprises a VH having about 70%, about 75%, about 80%, about 85%, about 90%, about 95%, about 96%, about 97%, about 98%, about 99%, or about 100% amino acid sequence identity to the sequence of a VH from an anti-CD19 disclosed in TABLE 2. In some aspects, the extracellular antigen recognition domain comprises a VL having about 70%, about 75%, about 80%, about 85%, about 90%, about 95%, about 96%, about 97%, about 98%, about 99%, or about 100% amino acid sequence identity to the sequence of a VL from an anti-CD19 disclosed in TABLE 2. In some aspects, the extracellular antigen recognition domain comprises a VH having about 70%, about 75%, about 80%, about 85%, about 90%, about 95%, about 96%, about 97%, about 98%, about 99%, or about 100% amino acid sequence identity to the sequence of a VH from an anti-CD19 disclosed in TABLE 2, and a VL having about 70%, about 75%, about 80%, about 85%, about 90%, about 95%, about 96%, about 97%, about 98%, about 99%, or about 100% amino acid sequence identity to the sequence of a VL from an anti-CD19 disclosed in TABLE 2.

In some aspects, the extracellular antigen recognition domain comprises a VH having about 70%, about 75%, about 80%, about 85%, about 90%, about 95%, about 96%, about 97%, about 98%, about 99%, or about 100% amino acid sequence identity to the sequence of a VH from an anti-CD20 disclosed in TABLE 2. In some aspects, the extracellular antigen recognition domain comprises a VL having about 70%, about 75%, about 80%, about 85%, about 90%, about 95%, about 96%, about 97%, about 98%, about 99%, or about 100% amino acid sequence identity to the sequence of a VL from an anti-CD20 disclosed in TABLE 2. In some aspects, the extracellular antigen recognition domain comprises a VH having about 70%, about 75%, about 80%, about 85%, about 90%, about 95%, about 96%, about 97%, about 98%, about 99%, or about 100% amino acid sequence identity to the sequence of a VH from an anti-CD20 disclosed in TABLE 2, and a VL having about 70%, about 75%, about 80%, about 85%, about 90%, about 95%, about 96%, about 97%, about 98%, about 99%, or about 100% amino acid sequence identity to the sequence of a VL from an anti-CD20 disclosed in the TABLE 2.

TABLE 2
CD19 and CD20 antibodies. Information about the antibodies in the table is available
from the International Immunogenics Information System (IMGT). The IMGT Acc. Nos.
disclosed in the present application correspond to the “INN Number” in
the IMGT/mAB-DB query available at www.imgt.org/mAb-DB/. HC: Heavy chain; LC:
Light Chain. For structures comprising multiple chimeric chains, e.g., the DART
Duvortuxizumab, polypeptides chains as identified as ChX wherein X is an integer.

| IMGT Acc. No. | Name | Target | SEQ ID NOS |
|---|---|---|---|
| 9028 | Blinatumomab | CD19 | 345 |
| 12546 | Budoprutug | CD19 | 346 (HC), 347 (LC) |
| 9558 | Coltuximab ravtansine | CD19 | 348 (HC), 349 (LC) |
| 9886 | Denintuzumab | CD19 | 350 (HC), 351 (LC) |
| 10506 | Duvortuxizumab | CD19 | 352 (Ch1), 353 (Ch2), 354 (Ch3) |
| 12023 | Emfizatamab | CD19 | 355 (HC), 356 (LC) |
| 11793 | Englumafusp alfa | CD19 | 357 (Ch1), 358 (Ch2), 359 (Ch3), 360 (Ch4) |
| 9985 | Inebilizumab | CD19 | 361 (HC), 362 (LC) |
| 10585 | Loncastuximab | CD19 | 363 (HC), 364 (LC) |
| 10898 | Obexelimab | CD19 | 365 (HC), 366 (LC) |
| 10835 | Tafasitamab | CD19 | 367 (HC), 368 (LC) |
| 8047 | Taplitumomab paptox | CD19 | 369 (HC), 370(LC) |
| 12219 | Zeripatamig | CD19 | 371 (Ch1), 372 (Ch2), 373 (Ch3) |
| 11336 | Divozilimab | CD20 | 374 (HC), 375 (LC) |
| 11078 | Epcoritamab | CD20 | 376 (Ch1), 377 (Ch2), 378 (Ch3), 379 (Ch4) |
| 7833 | Epitumomab cituxetan | CD20 | 380 (HC), 381 (LC) |
| 11590 | Eramkafusp alfa | CD20 | 382 (HC), 383 (LC) |
| 11145 | Glofitamab | CD20 | 384 (Ch1), 385 (Ch2), 386 (Ch3), 387 (Ch4) |
| 7873 | Ibritumomab tiuxetan | CD20 | 388 (HC), 389 (LC) |
| 12054 | Imvotamab | CD20 | 390 (Ch1), 391 (Ch2), 392 (Ch3) |
| 10621 | Mosunetuzumab | CD20 | 393 |
| 9043 | Obinutuzumab | CD20 | 394 (Ch1), 395 (Ch2), 396 (Ch3), 397 (Ch4) |
| 9590 | Ocaratuzumab | CD20 | 398 (HC), 399 (LC) |
| 8636 | Ocrelizumab | CD20 | 400 (Ch1), 401 (Ch2), 402 (Ch3) |
| 11035/8606 | Odronextamab/ofatumumab | CD20 | 403 (Ch1), 404 (Ch2), 405 (Ch3), 406 (Ch4) |
| 11020 | Plamotamab | CD20 | 407 (HC), 408 (LC) |
| 11212 | Ripertamab | CD20 | 409 (HC), 410 (LC) |
| 7609 | Rituximab | CD20 | 411 (HC), 412 (LC) |
| 7542 | Nofetumomab merpentan | CD20 | 413 (HC), 414 (LC) |
| 7827 | Tositumomab | CD20 | 415 (HC), 416 (LC) |
| 9334 | Ublituximab | CD20 | 417 (HC), 418 (LC) |
| 8932 | Veltuzumab | CD20 | 419 (HC), 420 (LC) |
| 11331 | Zuberitamab | CD20 | 421 (HC), 422 (LC) |

In some aspects, the CAR comprises, from N-terminus to C-terminus, the following operably linked elements: (1) an extracellular antigen recognition domain that, e.g., specifically binds to CD19 and CD20; (2) a spacer (e.g., a CD4 spacer or a CD28 spacer); (3) a transmembrane domain (e.g., CD4 or CD28 TM domain); (4) a 4-1BB activation domain; and, (5) a CD3zeta activation domain, and, optionally, costimulatory domain (e.g., a CD28 intracellular domain) interposed between elements (3) and (4).

In some aspects, the CAR comprises a full CAR presented in TABLE 3.

TABLE 3
Full sequences of anti-CD19/anti-CD20 bispecific CARs

| | | SEQ | |
|---|---|---|---|
| Full | Des- | ID | |
| CAR ID | cription | NO | SEQUENCE |
| MD2452 | AntiCD19/ | 423 | DIQLTQSPSFLSASVGDRVTITCKASQSVDYSGDSYLNWYQQKPGKAPKLLIYDASNLVSGVPSRFS |
| | AntiCD20 | | GSGSGTEFTLTISSLQPEDFATYYCQQSTENPWTFGGGTKLEIKGGSSGEIVLTQSPATLSLSPGER |
| | 2^{nd} | | ATLSCRASQSVSSYLAWYQQKPGQAPRLLIYDASNRATGIPARFSGSGSGTDFTLTISSLEPEDFAV |
| | generation | | YYCQQRSNWPITFGQGTRLEIKGGGGSGGSGSGGGGSASGSGEVQLVESGGGLVQPGRSLRLSCAAS |
| | Full | | GFTFNDYAMHWVRQAPGKGLEWVSTISWNSGSIGYADSVKGRFTISRDNAKKSLYLQMNSLRAEDTA |
| | sequence | | LYYCAKDIQYGNYYYGMDVWGQGTTVTVSSGGSSGQVQLVQSGAEVKKPGSSVKVSCKASGYAFSSY |
| | | | WMNWVRQAPGQGLEWMGQIWPGDSDTNYAQKFQGRVTITADESTSTAYMELSSLRSEDTAVYYCARR |
| | | | ETTTVGRYYYAMDYWGQGTTVTVSSASGGSGESNIKVLPTWSTPVQPMALIVLGGVAGLLLFIGLGI |
| | | | FFCVKRGRKKLLYIFKQPFMRPVQTTQEEDGCSCRFPEEEEGGCELRVKFSRSADAPAYQQGQNQLY |
| | | | NELNLGRREEYDVLDKRRGRDPEMGGKPRRKNPQEGLYNELQKDKMAEAYSEIGMKGERRRGKGHDG |
| | | | LYQGLSTATKDTYDALHMQALPPR |
| MD2740 | AntiCD19/ | 424 | DIQLTQSPSFLSASVGDRVTITCKASQSVDYSGDSYLNWYQQKPGKAPKLLIYDASNLVSGVPSRFS |
| | AntiCD20 | | GSGSGTEFTLTISSLQPEDFATYYCQQSTENPWTFGGGTKLEIKGGSSGEIVLTQSPATLSLSPGER |
| | 3^{rd} | | ATLSCRASQSVSSYLAWYQQKPGQAPRLLIYDASNRATGIPARFSGSGSGTDFTLTISSLEPEDFAV |
| | generation | | YYCQQRSNWPITFGQGTRLEIKGGGGSGGSGSGGGGSASGSGEVQLVESGGGLVQPGRSLRLSCAAS |
| | Full | | GFTFNDYAMHWVRQAPGKGLEWVSTISWNSGSIGYADSVKGRFTISRDNAKKSLYLQMNSLRAEDTA |
| | sequence | | LYYCAKDIQYGNYYYGMDVWGQGTTVTVSSGGSSGQVQLVQSGAEVKKPGSSVKVSCKASGYAFSSY |
| | | | WMNWVRQAPGQGLEWMGQIWPGDSDTNYAQKFQGRVTITADESTSTAYMELSSLRSEDTAVYYCARR |
| | | | ETTTVGRYYYAMDYWGQGTTVTVSSASGGSGSPLFPGPSKPFWVLVVVGGVLACYSLLVTVAFIIFW |
| | | | VRSKRSRLLHSDYMNMTPRRPGPTRKHYQPYAPPRDFAAYRSKRGRKKLLYIFKQPFMRPVQTTQEE |
| | | | DGCSCRFPEEEEGGCELRVKFSRSADAPAYQQGQNQLYNELNLGRREEYDVLDKRRGRDPEMGGKPR |
| | | | RKNPQEGLYNELQKDKMAEAYSEIGMKGERRRGKGHDGLYQGLSTATKDTYDALHMQALPPR |

In some aspects, the CAR comprises an extracellular antigen recognition domain presented in TABLE 4. In some aspects, the extracellular antigen recognition domain comprises a VH domain and a VL domain that specifically bind to CD19 and a VH domain that specifically binds to CD20. In some aspects, the anti-CD19 VH of the CAR has about 70%, about 75%, about 80%, about 85%, about 90%, about 95%, about 96%, about 97%, about 98%, about 99%, or about 100% amino acid sequence identity to the sequence of an anti-CD19 VH of an extracellular antigen recognition domain disclosed in TABLE 4. In some aspects, the anti-CD19 VL of the CAR has about 70%, about 75%, about 80%, about 85%, about 90%, about 95%, about 96%, about 97%, about 98%, about 99%, or about 100% amino acid sequence identity to the sequence of an anti-CD19 VL of an extracellular antigen recognition domain disclosed in TABLE 4.

In some aspects, the anti-CD20 VH of the CAR has about 70%, about 75%, about 80%, about 85%, about 90%, about 95%, about 96%, about 97%, about 98%, about 99%, or about 100% amino acid sequence identity to the sequence of an anti-CD20 VH of an extracellular antigen recognition domain disclosed in TABLE 4. In some aspects, the anti-CD20 VL of the CAR has about 70%, about 75%, about 80%, about 85%, about 90%, about 95%, about 96%, about 97%, about 98%, about 99%, or about 100% amino acid sequence identity to the sequence of an anti-CD20 VL of an extracellular antigen recognition domain disclosed in TABLE 4.

In some aspects, the anti-CD19 VH of the CAR has about 70%, about 75%, about 80%, about 85%, about 90%, about 95%, about 96%, about 97%, about 98%, about 99%, or about 100% amino acid sequence identity to the sequence of an anti-CD19 VH of an extracellular antigen recognition domain disclosed in TABLE 4; and the anti-CD19 VL of the CAR has about 70%, about 75%, about 80%, about 85%, about 90%, about 95%, about 96%, about 97%, about 98%, about 99%, or about 100% amino acid sequence identity to the sequence of an anti-CD19 VL of an extracellular antigen recognition domain disclosed in TABLE 4.

In some aspects, the anti-CD20 VH of the CAR has about 70%, about 75%, about 80%, about 85%, about 90%, about 95%, about 96%, about 97%, about 98%, about 99%, or about 100% amino acid sequence identity to the sequence of an anti-CD20 VH of an extracellular antigen recognition domain disclosed in TABLE 4; and the anti-CD20 VL of the CAR has about 70%, about 75%, about 80%, about 85%, about 90%, about 95%, about 96%, about 97%, about 98%, about 99%, or about 100% amino acid sequence identity to the sequence of an anti-CD20 VL of an extracellular antigen recognition domain disclosed in TABLE 4.

In some aspects, (i) the anti-CD19 VH of the CAR has about 70%, about 75%, about 80%, about 85%, about 90%, about 95%, about 96%, about 97%, about 98%, about 99%, or about 100% amino acid sequence identity to the sequence of an anti-CD19 VH of an extracellular antigen recognition domain disclosed in TABLE 4; (ii) the anti-CD19 VL of the CAR has about 70%, about 75%, about 80%, about 85%, about 90%, about 95%, about 96%, about 97%, about 98%, about 99%, or about 100% amino acid sequence identity to the sequence of an anti-CD19 VL of an extracellular antigen recognition domain disclosed in TABLE 4; (iii) the anti-CD20 VH of the CAR has about 70%, about 75%, about 80%, about 85%, about 90%, about 95%, about 96%, about 97%, about 98%, about 99%, or about 100% amino acid sequence identity to the sequence of an anti-CD20 VH of an extracellular antigen recognition domain disclosed in TABLE 4; and (iv) the anti-CD20 VL of the CAR has about 70%, about 75%, about 80%, about 85%, about 90%, about 95%, about 96%, about 97%, about 98%, about 99%, or about 100% amino acid sequence identity to the sequence of an anti-CD20 VL of an extracellular antigen recognition domain disclosed in TABLE 4.

TABLE 4
Extracellular antigen recognition domains and linker from anti-CD19/anti-CD20
bispecific CARs.

| Full CAR | | SEQ ID | |
|---|---|---|---|
| ID | Description | NO | SEQUENCE |
| MD2452 | Extracellular | 425 | DIQLTQSPSFLSASVGDRVTITCKASQSVDYSGDSYLNWYQQKPGKAPKLLIYDAS |
| MD2740 | antigen | | NLVSGVPSRFSGSGSGTEFTLTISSLQPEDFATYYCQQSTENPWTFGGGTKLEIKG |
| | recognition | | GSSGEIVLTQSPATLSLSPGERATLSCRASQSVSSYLAWYQQKPGQAPRLLIYDAS |
| | domain | | NRATGIPARFSGSGSGTDFTLTISSLEPEDFAVYYCQQRSNWPITFGQGTRLEIKG |
| | (VH and VL are | | GGGSGGSGSGGGGSASGSGEVQLVESGGGLVQPGRSLRLSCAASGFTFNDYAMHWV |
| | underlined) | | RQAPGKGLEWVSTISWNSGSIGYADSVKGRFTISRDNAKKSLYLQMNSLRAEDTAL |
| | | | YYCAKDIQYGNYYYGMDVWGQGTTVTVSSGGSSGQVQLVQSGAEVKKPGSSVKVSC |
| | | | KASGYAFSSYWMNWVRQAPGQGLEWMGQIWPGDSDTNYAQKFQGRVTITADESTST |
| | | | AYMELSSLRSEDTAVYYCARRETTTVGRYYYAMDYWGQGTTVTVSSASGGSG |
| MD2452 | Extracellular | 426 | GGGGSGGSGSGGGGSASGSG |
| MD2740 | antigen | | |
| | recognition | | |
| | domain linker | | |

In some aspects, the CAR comprises a CD4 spacer of sequence ESNIKVLPTWSTPVQPMA (SEQ ID NO: 515), a subsequence of human CD4 isoform 1 located between amino acid positions 381 and 298. In some aspects, the CAR comprises a CD28 spacer of sequence SPLFPGPSKP (SEQ ID NO: 516), a subsequence of human CD28 isoform 4 located between amino acid positions 157 and 166.

In some aspects, the CAR comprises a CAR spacer sequence disclosed in TABLE 5. In some aspects, the CAR spacer consists of a CAR spacer sequence disclosed in TABLE 5. In some aspects, the CAR spacer comprises a CAR spacer sequence disclosed in TABLE 5 plus 1, 2, 3, 4, 5, 6, 7, 8, 9, or 10 additional amino acids at the N-terminus. In some aspects, the CAR spacer comprises a CAR spacer sequence disclosed in TABLE 5 plus 1, 2, 3, 4, 5, 6, 7, 8, 9, or 10 additional amino acids at the C-terminus. In some aspects, the CAR spacer comprises a CAR spacer sequence disclosed in TABLE 5 plus 1, 2, 3, 4, 5, 6, 7, 8, 9, or 10 additional amino acids at the N-terminus, and 1, 2, 3, 4, 5, 6, 7, 8, 9, or 10 additional amino acids at the C-terminus.

In some aspects, the CAR spacer has about 70%, about 75%, about 80%, about 85%, about 90%, about 95%, about 96%, about 97%, about 98%, about 99%, or about 100% amino acid sequence identity to the sequence of a CAR spacer disclosed in TABLE 5.

In some aspects, the CAR spacer comprises a two or more sequences disclosed in TABLE 5, wherein the sequences are concatenated. In some aspects, each concatenated sequence is that same sequence from TABLE 5. In some aspects, each concatenated sequence is a different sequence from TABLE 5.

TABLE 5
CAR spacers (located between the extracellular antigen
recognition domain and transmembrane domain)

| CAR Spacers | Description | SEQ ID NO |
|---|---|---|
| CD4 spacer | CD4 isoform 1 381 . . . 398 | 427 |
| CD28 spacer | CD28 isoform 4 157 . . .. 166 | 428 |
| IgG2 spacer | IgG2 hinge derived | 429 |
| IgG2 spacer | IgG2 hinge derived | 430 |
| IgG2 spacer | IgG2 hinge derived | 431 |
| IgG3 spacer | IgG3 hinge derived | 432 |
| IgG3 spacer | IgG3 hinge derived | 433 |
| IgG3 spacer | IgG3 hinge derived | 434 |
| IgG3 spacer | IgG3 hinge derived | 435 |
| IgG3 spacer | IgG3 hinge derived | 436 |
| IgG3 spacer | IgG3 hinge derived | 437 |
| IgG3 spacer | IgG3 hinge derived | 438 |
| IgG1 spacer | IgG1 hinge derived | 439 |
| IgG3 spacer | IgG3 hinge derived | 440 |
| IgG2 spacer | IgG2 hinge derived | 441 |
| IgG4 spacer | IgG4 hinge derived | 442 |
| IgA1 spacer | IgA1 hinge derived | 443 |
| IgA1 spacer | IgA1 hinge derived | 444 |
| IgA1 spacer | IgA1 hinge derived | 445 |
| IgA1 spacer | IgA1 hinge derived | 446 |
| IgA1 spacer | IgA1 hinge derived | 447 |
| IgA1 spacer | IgA1 hinge derived | 448 |
| IgA1 spacer | IgA1 hinge derived | 449 |
| IgA1 spacer | IgA1 hinge derived | 450 |
| IgD spacer | IgD hinge derived | 451 |
| IgD spacer | IgD hinge derived | 452 |
| IgD spacer | IgD hinge derived | 453 |
| IgD spacer | IgD hinge derived | 454 |
| IgE spacer | IgE hinge derived | 455 |
| IgM spacer | IgM hinge derived | 456 |
| IgM spacer | IgM hinge derived | 457 |
| IgM spacer | IgM hinge derived | 458 |
| 41BB spacer | 41BB derived | 459 |
| 41BB spacer | 41BB derived | 460 |
| 41BB spacer | 41BB derived | 461 |
| CD8a spacer | CD8a derived | 462 |
| CD27 spacer | CD27 derived | 463 |
| CD27 spacer | CD27 derived | 464 |
| CD28 spacer | CD28 derived | 465 |
| IgG4 spacer | IgG4 derived | 466 |
| Dap10 spacer | Dap10 derived | 467 |
| IgG4 spacer | IgG4 derived | 468 |
| ICOS spacer | ICOS derived | 469 |
| OX40 spacer | OX40 derived | 470 |
| IgG4 spacer | IgG4 derived | 471 |
| IgG4 spacer | IgG4 CH2CH3 | 472 |
| IgG4 spacer | IgG4 hinge | 539 |

In some aspects, the CAR comprises a transmembrane region disclosed in TABLE 6. In some aspects, the transmembrane region comprises a CD4 transmembrane sequence (CD4 TM) disclosed in TABLE 6. In some aspects, the transmembrane region comprises a CD28 transmembrane sequence (CD28 TM) disclosed in TABLE 6. In some aspects, the transmembrane region has about 70%, about 75%, about 80%, about 85%, about 90%, about 95%, about 96%, about 97%, about 98%, about 99%, or about 100% amino acid sequence identity to the sequence of a transmembrane region disclosed in TABLE 6.

In some aspects, the CAR comprises an intracellular region components or combination thereof disclosed in TABLE 6. In some aspects, the CAR comprises a CD28 intracellular domain disclosed in TABLE 6. In some aspects, the CAR comprises a 4-1BB activation domain disclosed in TABLE 6. In some aspects, the CAR comprises a CD3zeta (CD3ζ) activation domain disclosed in TABLE 6. In some aspects, the CAR comprises a transmembrane region and intracellular region (TM+IC region) disclosed in TABLE 6. In some aspects, the CAR comprises an intracellular region (IC region) disclosed in TABLE 6.

In some aspects, the CAR comprises a CD28 intracellular domain having about 70%, about 75%, about 80%, about 85%, about 90%, about 95%, about 96%, about 97%, about 98%, about 99%, or about 100% amino acid sequence identity to the sequence of a CD28 intracellular domain disclosed in TABLE 6.

In some aspects, the CAR comprises a 4-1BB activation domain having about 70%, about 75%, about 80%, about 85%, about 90%, about 95%, about 96%, about 97%, about 98%, about 99%, or about 100% amino acid sequence identity to the sequence of a 4-1BB activation domain disclosed in TABLE 6.

In some aspects, the CAR comprises a CD3zeta (CD3ζ) activation domain having about 70%, about 75%, about 80%, about 85%, about 90%, about 95%, about 96%, about 97%, about 98%, about 99%, or about 100% amino acid sequence identity to the sequence of a CD3zeta (CD3ζ) activation domain disclosed in TABLE 6.

In some aspects, the CAR comprises a transmembrane region and intracellular region (TM+IC region) having about 70%, about 75%, about 80%, about 85%, about 90%, about 95%, about 96%, about 97%, about 98%, about 99%, or about 100% amino acid sequence identity to the sequence of TM+IC region disclosed in TABLE 6.

In some aspects, the CAR comprises an intracellular region (IC region) having about 70%, about 75%, about 80%, about 85%, about 90%, about 95%, about 96%, about 97%, about 98%, about 99%, or about 100% amino acid sequence identity to the sequence of an IC region disclosed in TABLE 6.

TABLE 6
Transmembrane regions and intracellular regions of CARs.

| TM/IC regions | Description | SEQ ID NO |
|---|---|---|
| CD4 TM | Protein | 517 |
| CD28 TM | Nucleic acid | 518 |
| CD28 TM | Protein | 519 |
| CD28 IC | Nucleic acid | 520 |
| CD28 IC | Protein | 521 |
| 4-1BB activation domain | Nucleic acid | 522 |
| 4-1BB activation domain | Protein | 523 |
| CD3zeta (CD3ζ) activation domain | Nucleic acid | 524 |
| CD3zeta (CD3ζ) activation domain | Protein | 525 |
| TM + IC region | 2^{nd} generation CAR | 526 |
| TM + IC region | 3td generation CAR | 527 |
| IC region | 2^{nd} generation CAR | 528 |
| IC region | 3^{rd} generation CAR | 529 |

### I.B.iv Payload: Therapeutic Antibodies

In some aspects, the payload comprises a therapeutic antibody or an antigen-binding portion thereof, e.g., a therapeutic antibody disclosed below or an antigen-binding portion thereof. In some aspects the payload can comprise a fusion protein comprising a therapeutic antibody disclosed below or an antigen-binding portion thereof.

It is to be noted that the used of the therapeutic antibodies and antigen-binding portions thereof disclose below is not limited to serve as payloads in the delivery systems of the present disclosure. Thus, in some aspects, a therapeutic antibody disclosed below or an antigen-binding portion thereof can be or can be part of a surface anchored targeting molecule in a LNP delivery system of the present disclosure, and direct the LNP to a cell or tissue expressing the molecule to which an antibody disclosed below binds specifically.

In some aspects, VH and/or VL domains of antibodies disclosed below can be used as part of the targeting portion of a CAR. In some aspects, a CAR used as payload in a LNP delivery system of the present disclosure can comprise a scFv comprising VH and VL domains from any of the therapeutic antibodies disclosed below.

In some aspects, the payload comprises an antibody or antigen-binding portion thereof selected from the group consisting of 3F8 (anti-GD2 ganglioside), abagovomab (anti-CA-125), abciximab (anti-CD41, integrin alpha-IIb), abituzumab (anti-CD51), abrezekimab (anti-IL-13), abrilumab (anti-integrin α4 β7), actoxumab (anti-Clostridium difficile), adalimumab (anti-TNF-α), adecatumumab (anti-EpCAM), aducanumab (anti-Amyloid beta), afasevikumab (anti-IL-17A, IL-17F), afelimomab (anti-TNF-α), alacizumab pegol[13]VEGFR2), alemtuzumab (anti-CD52), alirocumab (anti-PCSK9), altumomab pentetate (anti-carcinoembryonic antigen (CEA)), amatuximab[(anti-mesothelin), amivantamab (anti-epidermal growth factor receptor (EGFR), cMet), anatumomab mafenatox (anti-tumor-associated glycoprotein 72 (TAG-72)), andecaliximab (anti-gelatinase B), anetumab ravtansine (anti-mesothelin (MSLN)), anifrolumab (anti-IFN-α/β receptor), ansuvimab (anti-Ebola virus glycoprotein), anrukinzumab (anti-IL-13), apolizumab[(anti-HLA-DR), aprutumab ixadotin (anti-FGFR2), arcitumomab (anti-Carcinoembryonic antigen (CEA)), ascrinvacumab (anti-activin receptor-like kinase 1), aselizumab (anti-L-selectin (CD62L)), atezolizumab (anti-PD-L1), atidortoxumab (anti-Staphylococcus aureus alpha toxin), atinumab (anti-RTN4), atorolimumab (anti-Rhesus factor), avelumab (anti-PD-L1), azintuxizumab vedotin (anti-CD319), bamlanivimab (anti-spike protein receptor binding domain (RBD) of SARS-CoV-2), bapineuzumab (anti-β-amyloid), basiliximab (anti-CD25 (a chain of IL-2 receptor)), bavituximab (anti-phosphatidylserine), BCD-100 (anti-PD-1), bebtelovimab (anti-spike protein receptor binding domain (RBD) of SARS-CoV-2), bectumomab (anti-CD22), bedinvetmab (anti-nerve growth factor (NGF)), begelomab (anti-DPP4), belantamab mafodotin (anti-B-cell maturation antigen (BCMA)), belimumab (anti-B-cell activating factor (BAFF)), bemarituzumab (anti-FGFR2), benralizumab[(anti-CD125), berlimatoxumab (anti-Staphylococcus aureus bi-component leucocidin), bermekimab (anti-IL-1α), bersanlimab (anti-ICAM-1), bertilimumab (anti-CCL11 (eotaxin-1)), besilesomab (anti-carcinoembryonic antigen (CEA)-related antigen), bevacizumab (anti-VEGF-A), bezlotoxumab[(anti-Clostridium difficile), biciromab (anti-beta chain), bimagrumab (anti-ACVR2B), bimekizumab (anti-IL-17A, IL-17F, IL-17AF), birtamimab (anti-serum amyloid A protein), bivatuzumab (anti-CD44 v6), bleselumab (anti-CD40), blinatumomab (anti-CD19), blontuvetmab (anti-CD20), blosozumab (anti-SOST), bococizumab (anti-PCSK9), brazikumab (anti-IL-23), brentuximab vedotin (anti-CD30 (TNFRSF8)), briakinumab (anti-IL-12, IL-23), brodalumab (anti-IL-17), brolucizumab (anti-vascular endothelial growth factor A (VEGFA)), brontictuzumab (anti-Notch 1), burosumab (anti-FGF 23), cabiralizumab (anti-CSF1R), camidanlumab tesirine (anti-CD25 (a chain of IL-2 receptor), camrelizumab (anti-PD-1), canakinumab (anti-IL-1), cantuzumab mertansine (anti-CanAg (a glycoform of MUC1)), cantuzumab ravtansine (anti-CanAg (a glycoform of MUC1)), caplacizumab (anti-VWF), casirivimab (anti-spike protein receptor binding domain (RBD) of SARS-CoV-2), capromab (anti-Glutamate carboxypeptidase II), carlumab (anti-MCP-1), carotuximab (anti-endoglin), catumaxomab (anti-EpCAM, CD3), cBR96-doxorubicin immunoconjugate (anti-Lewis-Y antigen), cedelizumab (anti-CD4), cemiplimab (anti-PD-1), cergutuzumab amunaleukin (anti-IL-2), certolizumab pegol (anti-TNF-α), cetrelimab (anti-PD-1), cetuximab (anti-epidermal growth factor receptor (EGFR)), cibisatamab (anti-CEACAM5), cilgavimab (anti-spike protein receptor binding domain (RBD) of SARS-CoV-2), cirmtuzumab (anti-ROR1), citatuzumab bogatox (anti-EpCAM), cixutumumab (anti-IGF-1 receptor (CD221)), clazakizumab (anti-IL-6), clenoliximab (anti-CD4), clivatuzumab tetraxetan (anti-MUC1), codrituzumab (anti-glypican 3), cofetuzumab pelidotin (anti-PTK7), coltuximab ravtansine (anti-CD19), conatumumab (anti-TRAIL-R2), concizumab (anti-tissue factor pathway inhibitor (TFPI)), cosfroviximab (anti-ebolavirus glycoprotein), crenezumab (anti-β-amyloid (1-40 and 1-42)), crizanlizumab (anti-selectin P), crotedumab (anti-glucagon receptor (GCGR)), CR6261 (anti-Hemagglutinin (influenza)), cusatuzumab (anti-CD70), dacetuzumab (anti-CD40), daclizumab (anti-CD25 (a chain of IL-2 receptor), dalotuzumab (anti-IGF-1 receptor (CD221)), dapirolizumab pegol (anti-CD154 (CD40L)), daratumumab (anti-CD38), dectrekumab (anti-IL-13), demcizumab (anti-DLL4), denintuzumab mafodotin (anti-CD19), denosumab (anti-RANKL), depatuxizumab mafodotin (anti-EGFR), derlotuximab biotin (anti-histone complex), detumomab (anti-B-lymphoma cell), dezamizumab (anti-serum amyloid P component), dinutuximab (anti-GD2 ganglioside), dinutuximab beta (anti-GD2 ganglioside), diridavumab (anti-Hemagglutinin (influenza)), domagrozumab (anti-GDF-8), donanemab (anti-Amyloid beta), dostarlimab (anti-PCDP1), drozitumab (anti-DR5), DS-8201 (anti-HER2), duligotuzumab (anti-ERBB3 (HER3)), dupilumab (anti-IL-4Ra), durvalumab (anti-PD-L1), dusigitumab (anti-IGF-2), duvortuxizumab (anti-CD19, CD3E), ecromeximab (anti-GD3 ganglioside), eculizumab (anti-C5), edobacomab (anti-endotoxin), edrecolomab (anti-EpCAM), efalizumab (anti-LFA-1 (CD11a)), efungumab (anti-Hsp90), eldelumab (anti-CXCL10 (IP-10)), elezanumab (anti-repulsive guidance molecule A (RGMA)), elgemtumab (anti-ERBB3 (HER3)), elotuzumab (anti-SLAMF7), elsilimomab (anti-IL-6), emactuzumab (anti-CSF1R), emapalumab (anti-IFN-γ), emibetuzumab (anti-HGFR), emicizumab (anti-activated F9, F10), enapotamab vedotin (anti-AXL), enavatuzumab (anti-TWEAK receptor), enfortumab vedotin (anti-nectin-4), enlimomab pegol (anti-ICAM-1 (CD54)), enoblituzumab (anti-CD276), enokizumab (anti-IL-9), enoticumab (anti-DLL4), ensituximab (anti-MUC5AC), epcoritamab (anti-CD3, CD20), epitumomab cituxetan (anti-episialin), epratuzumab (anti-CD22), eptinezumab (anti-calcitonin gene-related peptide), erenumab (anti-calcitonin gene-related peptide receptor (CGRP)), erlizumab (anti-ITGB2 (CD18)), ertumaxomab (anti-HER2/neu, CD3), etaracizumab (anti-integrin αvβ3), etesevimab (anti-spike protein receptor binding domain (RBD) of SARS-CoV-2), etigilimab (anti-TIGIT), etrolizumab (anti-integrin β7), evinacumab (anti-angiopoietin 3), evolocumab (anti-PCSK9), exbivirumab (anti-hepatitis B surface antigen), fanolesomab(anti-CD15), faralimomab (anti-IFN receptor), faricimab (anti-VEGF-A and Ang-2), farletuzumab (anti-folate receptor 1), fasinumab (anti-nerve growth factor (NGF)), FBTA05 (anti-CD20), felvizumab (anti-respiratory syncytial virus), fezakinumab (anti-IL-22), fibatuzumab (anti-ephrin receptor A3), ficlatuzumab (anti-Hepatocyte growth factor (HGF)), figitumumab (anti-IGF-1 receptor (CD221)), firivumab (anti-Hemagglutinin (influenza)), flanvotumab (anti-TYRP1 (glycoprotein 75)), fletikumab (anti-IL-20), flotetuzumab (anti-IL-3 receptor), fontolizumab (anti-IFN-γ), foralumab (anti-CD3E), foravirumab (anti-rabies virus glycoprotein), fremanezumab (anti-calcitonin gene-related peptide alpha and beta), fresolimumab (anti-TGF-β), frovocimab (anti-PCSK9), frunevetmab (anti-nerve growth factor (NGF)), fulranumab (anti-nerve growth factor (NGF)), futuximab (anti-Epidermal growth factor receptor (EGFR)), galcanezumab (anti-calcitonin), galiximab (anti-CD80), gancotamab (anti-HER2/neu), ganitumab (anti-IGF-1 receptor (CD221)), gantenerumab (anti-β-amyloid (1-40 and 1-42)), gatipotuzumab (anti-MUC1), gavilimomab (anti-CD147 (basigin)), gedivumab (anti-Hemagglutinin (influenza)), gemtuzumab ozogamicin (anti-CD33), gevokizumab (anti-IL-10), gilvetmab (anti-PCDC1), gimsilumab (anti-CSF2), girentuximab (anti-carbonic anhydrase 9 (CA-IX)), glembatumumab vedotin (anti-GPNMB), glofitamab (anti-CD20, CD3), golimumab (anti-TNF-α), gomiliximab (anti-CD23 (IgE receptor), gosuranemab (anti-tau protein), guselkumab (anti-IL-23), ianalumab (anti-BAFF-R), ibalizumab (anti-CD4), sintilimab (anti-PD-1), ibritumomab tiuxetan (anti-CD20), icrucumab (anti-VEGFR-1), idarucizumab (anti-dabigatran), ifabotuzumab (anti-EPHA3), igovomab (anti-CA-125), iladatuzumab vedotin (anti-CD79B), imalumab (anti-macrophage migration inhibitory factor (MIF)), imaprelimab (anti-melanoma cell adhesion molecule (MCAM)), imciromab (anti-cardiac myosin), imdevimab (anti-spike protein receptor binding domain (RBD) of SARS-CoV-2), imgatuzumab (anti-Epidermal growth factor receptor (EGFR)), inclacumab (anti-selectin P), indatuximab ravtansine (anti-SDC1), indusatumab vedotin (anti-GUCY2C), inebilizumab (anti-CD19), infliximab (anti-TNF-α), intetumumab (anti-CD51), inolimomab (anti-CD25 (a chain of IL-2 receptor)), inotuzumab ozogamicin (anti-CD22), ipilimumab (anti-CD152), iomab-B (anti-CD45), iratumumab (anti-CD30 (TNFRSF8)), isatuximab (anti-CD38), iscalimab (anti-CD40), istiratumab (anti-IGF-1 receptor (CD221)), itolizumab (anti-CD6), ixekizumab (anti-IL-17A), keliximab (anti-CD4), labetuzumab (anti-Carcinoembryonic antigen (CEA)), lacnotuzumab (anti-CSF1, macrophage colony stimulating factor (MCSF)), ladiratuzumab vedotin (anti-LIV-1), lampalizumab (anti-Complement factor D (CFD)), lanadelumab (anti-kallikrein), landogrozumab (anti-GDF-8), laprituximab emtansine (anti-epidermal growth factor receptor (EGFR)), larcaviximab (anti-ebolavirus glycoprotein), lebrikizumab (anti-IL-13), lecanemab (anti-β-amyloid), lemalesomab (anti-NCA-90 (granulocyte antigen)), lendalizumab (anti-C5), lenvervimab (anti-hepatitis B surfage antigen), lenzilumab (anti-CSF2), lerdelimumab (anti-TGF-02), leronlimab (anti-CCR5), lesofavumab (anti-Hemagglutinin (influenza)), letolizumab (anti-tumor necrosis factor related activation protein (TRAP)), lexatumumab (anti-TRAIL-R2), libivirumab (anti-hepatitis B surface antigen), lifastuzumab vedotin (anti-phosphate-sodium co-transporter), ligelizumab (anti-IGHE), loncastuximab tesirine (anti-CD19), losatuxizumab vedotin (anti-EGRF, ERBB1 HER1), lilotomab satetraxetan (anti-CD37), lintuzumab (anti-CD33), lirilumab (anti-KIR2D), lodelcizumab (anti-PCSK9), lorvotuzumab mertansine (anti-CD56), lucatumumab (anti-CD40), lulizumab pegol (anti-CD28), lumiliximab (anti-CD23 (IgE receptor)), lumretuzumab (anti-ERBB3 (HER3)), lupartumab amadotin (anti-LYPD3), lutikizumab (anti-IL-1α), mapatumumab (anti-TRAIL-R1), margetuximab (anti-HER2), marstacimab (anti-tissue factor pathway inhibitor (TFPI)), maslimomab (anti-T cell receptor), mavrilimumab (anti-GMCSF receptor α-chain), matuzumab (anti-epidermal growth factor receptor (EGFR)), mepolizumab (anti-IL-5), metelimumab (anti-TGF-01), milatuzumab (anti-CD74), minretumomab (anti-TAG-72), mirikizumab (anti-IL-23), mirvetuximab soravtansine (anti-folate receptor alpha), mitumomab (anti-GD3 ganglioside), modotuximab (anti-EGFR extracellular domain III), mogamulizumab (anti-CCR4), monalizumab (anti-NKG2A), morolimumab (anti-Rhesus factor), mosunetuzumab (anti-CD3E, MS4A1, CD20), motavizumab (anti-respiratory syncytial virus), moxetumomab pasudotox (anti-CD22), muromonab-CD3 (anti-CD3), nacolomab tafenatox (anti-C242 antigen), namilumab (anti-CSF2), naptumomab estafenatox (anti-5T4)), naratuximab emtansine (anti-CD37)), narnatumab (anti-MST1R (aka RON)), natalizumab (anti-integrin α4), navicixizumab (anti-DLL4 and VEGFA), navivumab (anti-Hemagglutinin (influenza)), naxitamab (anti-c-Met), nebacumab (anti-endotoxin), necitumumab (anti-epidermal growth factor receptor (EGFR)), nemolizumab (anti-IL-31 receptor A), NEOD001 (anti-amyloid), nerelimomab (anti-TNF-α, nesvacumab (anti-angiopoietin 2), netakimab (anti-IL-17A), nimotuzumab (anti-epidermal growth factor receptor (EGFR)), nirsevimab (anti-RSV fusion glycoprotein), nivolumab (anti-PD-1), nofetumomab merpentan (pancarcinoma murine antibody NR-LU-10 linked with gamma-emitting radioisotope technetium 99m (Tc 99m)), obiltoxaximab (anti-Bacillus anthracis anthrax), obinutuzumab anti-CD20, ocaratuzumab (anti-CD20), ocrelizumab (anti-CD20). atoltivimab/maftivimab/odesivimab (INMAZEB®, REGN-EB3) (anti-Zaire ebolavirus glycoprotein combination therapy), odulimomab (anti-LFA-1 (CD11a)), ofatumumab (anti-CD20), olaratumab (anti-PDGFRA), oleclumab (anti-5′-nucleotidase), olendalizumab (anti-complement C5a), olokizumab (anti-IL-6), omalizumab (anti-IgE Fc region), omburtamab (anti-CD276), oMS721 (anti-MASP-2), onartuzumab (anti-human scatter factor receptor kinase), ontuxizumab (anti-TEM1), onvatilimab (anti-VISTA (protein) (VSIR)); opicinumab (anti-LINGO-1), oportuzumab monatox (anti-EpCAM), oregovomab (anti-CA-125), orticumab (anti-oxLDL), otelixizumab (anti-CD3), otilimab (anti-GMCSF), otlertuzumab (anti-CD37), oxelumab (anti-OX-40), ozanezumab (anti-NOGO-A), ozoralizumab (anti-TNF-α), pagibaximab (anti-lipoteichoic acid), palivizumab (anti-F protein of respiratory syncytial virus), pamrevlumab (anti-connective tissue growth factor (CTGF)), panitumumab (anti-epidermal growth factor receptor (EGFR)), pankomab (anti-tumor specific glycosylation of MUC1), panobacumab (anti-Pseudomonas aeruginosa), parsatuzumab (anti-EGFL7), pascolizumab (anti-IL-4), pasotuxizumab (anti-folate hydrolase), pateclizumab (anti-lymphotoxin alpha (LTA)), patritumab (anti-ERBB3 (HER3)), PDR001 (anti-PD-1), pembrolizumab (anti-PD-1), pemtumomab (anti-MUC1), perakizumab (anti-IL-17A), pertuzumab (anti-HER2/neu), pexelizumab (anti-C5), pidilizumab (anti-PD-1), pinatuzumab vedotin (anti-CD22), pintumomab (anti-adenocarcinoma antigen), placulumab (anti-TNF), pozelimab (anti-C5), prezalumab (anti-TNF), plozalizumab (anti-CCR2), pogalizumab (anti-tumor necrosis factor receptor (TNFR) superfamily member 4), polatuzumab vedotin (anti-CD79B), ponezumab (anti-β-amyloid), porgaviximab (anti-Zaire ebolavirus glycoprotein), prasinezumab (anti-Alpha-synuclein), prezalizumab (anti-inducible T cell co-stimulatory ligand (ICOSL)), priliximab (anti-CD4), pritoxaximab (anti-E. coli shiga toxin type-1), pritumumab (anti-vimentin), PRO 140 (anti-CCR5), quilizumab (anti-IGHE), racotumomab (anti-NGNA ganglioside), radretumab (anti-fibronectin extra domain-B), rafivirumab (anti-rabies virus glycoprotein), ralpancizumab (anti-PCSK9), ramucirumab (anti-VEGFR2), ranevetmab (anti-NGF), ranibizumab (anti-VEGF-A), raxibacumab (anti-anthrax toxin protective antigen), ravagalimab (anti-CD40), ravulizumab (anti-C5), refanezumab (anti-myelin-associated glycoprotein), regavirumab (anti-cytomegalovirus glycoprotein B), regdanvimab (anti-spike protein receptor binding domain (RBD) of SARS-CoV-2), relatlimab (anti-LAG3), remtolumab (anti-IL-17A, TNF), reslizumab (anti-IL-5), retifanlimab (anti-PD-1), rilotumumab (anti-hepatocyte growth factor (HGF)), rinucumab (anti-PDGFRB), risankizumab (anti-IL-23A), rituximab (anti-CD20), rivabazumab pegol (anti-Pseudomonas aeruginosa type III secretion system), robatumumab (anti-IGF-1 receptor (CD221)), rmab (anti-rabies virus G glycoprotein), roledumab (anti-RHD (gene) (RHD)), romilkimab (anti-IL-13), romosozumab (anti-sclerostin), rontalizumab (anti-IFN-α), rosmantuzumab (anti-root plate-specific spondin 3), rovalpituzumab tesirine (anti-DLL3), rovelizumab (anti-CD11, anti-CD18), rozanolixizumab (anti-FCGRT), ruplizumab (anti-CD154 (CD40L)), SA237 (anti-IL-6 receptor), sacituzumab govitecan (anti-TROP-2), samalizumab (anti-CD200), samrotamab vedotin (anti-LRRC15), sarilumab (anti-IL-6), satralizumab (anti-IL-6 receptor), satumomab pendetide (anti-TAG-72), secukinumab (anti-IL-17A), selicrelumab (anti-CD40), seribantumab (anti-ERBB3 (HER3)), setoxaximab (anti-E. coli shiga toxin type-2), setrusumab (anti-sclerostin (SOST)), sevirumab (anti-cytomegalovirus), sibrotuzumab (anti-FAP (gene) (FAP)), SGN-CD19A (anti-CD19), SHP647 (anti-mucosal addressin cell adhesion molecule), sifalimumab (anti-IFN-α), siltuximab (anti-IL-6), simtuzumab (anti-LOXL2), siplizumab (anti-CD2), sirtratumab vedotin (anti-SLITRK6), sirukumab (anti-IL-6), sofituzumab vedotin (anti-CA-125), solanezumab (anti-β-amyloid), solitomab (anti-EpCAM), sonepcizumab (anti-sphingosine-1-phosphate), sontuzumab (anti-episialin), sotrovimab (anti-spike protein receptor binding domain (RBD) of SARS-CoV-2), spartalizumab (anti-PD-1), spesolimab (anti-Interleukin 36 receptor (IL1RL2/IL1RAP)), stamulumab (anti-myostatin), sulesomab (anti-NCA-90 (granulocyte antigen)), suptavumab (anti-RSVFR), sutimlimab (anti-complement component is (CIs)), suvizumab (anti-HIV-1), suvratoxumab (anti-Staphylococcus aureus alpha toxin), tabalumab (anti-B-cell activating factor (BAFF)), tacatuzumab tetraxetan (anti-alpha-fetoprotein), tadocizumab (anti-integrin αIIbβ3), tafasitamab (anti-CD19), talacotuzumab (anti-CD123), talizumab (anti-IgE), talquetamab (anti-GPRC5D, anti-CD3), tamtuvetmab (anti-CD52), tanezumab (anti-nerve growth factor (NGF)), taplitumomab paptox (anti-CD19), tarextumab (anti-Notch receptor), tavolimab (anti-CD134), teclistamab (anti-B-cell maturation antigen (BCMA), CD3), tefibazumab (anti-clumping factor A), telimomab aritox (anti-CD5), telisotuzumab (anti-HGFR), telisotuzumab vedotin (anti-HGFR), tenatumomab (anti-tenascin C), teneliximab (anti-CD40), teplizumab (anti-CD3), tepoditamab (anti-dendritic cell-associated lectin 2), teprotumumab (anti-IGF-1 receptor (CD221)), tesidolumab (anti-C5), tetulomab (anti-CD37), tezepelumab (anti-thymic stromal lymphopoietin (TSLP)), TGN1412 (anti-CD28), tibulizumab (anti-B-cell activating factor (BAFF)), tildrakizumab (anti-IL-23), tigatuzumab (anti-TRAIL-R2), timigutuzumab (anti-HER2), timolumab (anti-AOC3), tiragotumab (anti-TIGIT), tislelizumab (anti-PCDC1, anti-CD279), tisotumab vedotin (anti-coagulation factor III), tixagevimab (anti-spike protein receptor binding domain (RBD) of SARS-CoV-2), TNX-650 (anti-IL-13), tocilizumab (anti-IL-6 receptor), tomuzotuximab (anti-Epidermal growth factor receptor (EGFR), anti-HER1), toralizumab (anti-CD154 (CD40L)), tosatoxumab (anti-Staphylococcus aureus), tositumomab (anti-CD20), tovetumab (anti-PDGFRA), tralokinumab (anti-IL-13), trastuzumab (anti-HER2/neu), trastuzumab duocarmazine (anti-HER2/neu), trastuzumab emtansine (anti-HER2/neu), TRBS07 (anti-GD2 ganglioside), tregalizumab (anti-CD4), tremelimumab (anti-CTLA-4), trevogrumab (anti-growth differentiation factor 8), tucotuzumab celmoleukin (anti-EpCAM), tuvirumab (anti-hepatitis B virus), ublituximab (anti-CD20), ulocuplumab (anti-CXCR4 (CD184)), urelumab (anti-4-1BB (CD137)), urtoxazumab (anti-Escherichia coli), ustekinumab (anti-IL-12, anti-IL-23), utomilumab (anti-4-1BB (CD137)), vadastuximab talirine (anti-CD33), vanalimab (anti-CD40), vandortuzumab vedotin (anti-STEAP1), vantictumab (anti-Frizzled receptor), vanucizumab (anti-angiopoietin 2), vapaliximab (anti-AOC3/VAP-1), varisacumab (anti-VEGF-A), varlilumab (anti-CD27), vatelizumab (anti-ITGA2/CD49b), vedolizumab (anti-integrin α4 β7), veltuzumab (anti-CD20), vepalimomab (anti-AOC3, a.k.a., VAP-1), vesencumab (anti-NRP1), vilobelimab (anti-C5a receptor; C5a), visilizumab (anti-CD3), vobarilizumab (anti-IL-6 receptor), volociximab (anti-integrin α5β1), vonlerolizumab (anti-CD134), vopratelimab (anti-CD278, a.k.a. ICOS), vorsetuzumab mafodotin (anti-CD70), votumumab (anti-tumor antigen CTAA16.88), vunakizumab (anti-IL-17A), xentuzumab (anti-IGF-1, anti-IGF-2), XMAB-5574 (anti-CD19), zalutumumab (anti-Epidermal growth factor receptor, EGFR), zanolimumab (anti-CD4), zatuximab (an anti-HER1), zenocutuzumab (anti-ERBB3/HER3), ziralimumab (anti-CD147/basigin), zolbetuximab (anti-claudin 18 isoform 2), and zolimomab aritox (anti-CD5).

In some aspects, the payload comprises an antibody or antigen-binding portion thereof targets an apoptosis regulator. In some aspects, the apoptosis regulator is pro-apoptotic gene product, e.g, FasL (Fas ligand), BAX, BID, BAK or BAD. In some aspects, the apoptosis regulator is an anti-apoptotic gene product, e.g, Bcl-XI, an IAP (e.g., XIAP), or Bcl-2. In some aspects, the apoptosis regulator is a prosurvival factor such as cFLIP, BNIP3, FADD, Akt, or NF-κB.

### I.B.v Payload: Therapeutic Oligonucleotides.

In some aspects, the biologically active molecule (payload) is a nucleic acid, e.g., an RNA or a DNA. Nucleic acid active agents suitable for delivery using the LNP of the present disclosure include all types of RNA and all types of DNA, including also oligonucleotides such as probes and primers used in the polymerase chain reaction (PCR), hybridizations, or DNA sequencing. In some aspects, the nucleic acid comprises mRNA, miRNA, miRNA sponge, tough decoy miRNA (TD), antimir (antagomir), small RNA, rRNA, siRNA, shRNA, gDNA, cDNA, pDNA, PNA, BNA, antisense oligonucleotide (ASO), aptamer, cyclic dinucleotide, or any combination thereof.

In some aspects, the biologically active molecule (payload) comprises a short interfering RNA (siRNA), which is a double-stranded RNA that can induce sequence-specific post-transcriptional gene silencing, thereby decreasing or even inhibiting gene expression. For example, siRNAs can trigger the specific degradation of homologous RNA molecules, such as mRNAs, within the region of sequence identity between both the siRNA and the target RNA. Non-limiting exemplary siRNAs are disclosed in WO 02/44321, which is incorporated by reference in its entirety.

In some aspects, the LNP of the present disclosure is a targeted (conjugated) LNP wherein the payload is an mRNA. In some aspects, the LNP of the present disclosure is a non-targeted (non-conjugated) LNP wherein the payload is an mRNA. In some aspects, the targeted (conjugated) LNP can be administered to a human subject at a dose range between about 0.02 mg/kg and about 0.2 mg/kg, e.g., about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, or about 0.20 mg/kg.

In some aspects, the biologically active molecule (payload) comprises a short hairpin RNAs (shRNAs). In some aspects, the biologically active molecule comprises a miRNA or a miRNA inhibitor (antimiR). In some aspects, the biologically active molecule (payload) can be between about 10 and about 30 nucleotides in length, for example from about 14 to about 25 nucleotides in length. In some aspects, the biologically active molecule (payload) has a length of 16 to 30 nucleotides, 18 to 25 nucleotides, particularly 18, 19, 20, 21, 22, 23, 24, or 25 nucleotides in length.

Sequences for miRNAs are available publicly, for example, through the miRBase registry (Griffiths-Jones, et al., Nucleic Acids Res., 36 (Database Issue):D154-D158 (2008); Griffiths-Jones, et al., Nucleic Acids Res., 36 (Database Issue):D140-D144 (2008); Griffiths-Jones, et al., Nucleic Acids Res., 36 (Database Issue):D109-D111 (2008)) and other publically accessible databases.

In some aspects, the miRNA inhibitors are oligomers or polymers of ribonucleic acid (RNA) or deoxyribonucleic acid (DNA) or modifications thereof. In some aspects, the miRNA antagonists are antimir. Antimirs are a specific class of miRNA inhibitors that are described, for example, in US2007/0213292 to Stoffel et al. Antimirs are RNA-like oligonucleotides that contain various modifications for RNase protection and pharmacologic properties such as enhanced tissue and cellular uptake. Antimirs differ from normal RNA by having complete 2′-O-methylation of sugar, phosphorothioate backbone and a cholesterol-moiety at 3′-end.

Non-limiting examples of antimirs and other miRNA inhibitors are described in WO2009/020771, WO2008/091703, WO2008/046911, WO2008/074328, WO2007/090073, WO2007/027775, WO2007/027894, WO2007/021896, WO2006/093526, WO2006/112872, WO2007/112753, WO2007/112754, WO2005/023986, or WO2005/013901, all of which are hereby incorporated by reference.

In some aspects, the nucleic acids are phosphodiester antisense oligonucleotides, and any oligonucleotides where the sugar-phosphate “backbone” has been derivatized or replaced with “backbone analogues” such as with phosphorothioate, phosphorodithioate, phosphoroamidate, alkyl phosphotriester, or methylphosphonate linkages. In some aspects, the nucleic acids active agents are antisense oligonucleotides, and any oligonucleotides or oligodeoxynucleotides with non-phosphorous backbone analogues such as sulfamate, 3′-thioformacetal, methylene(methylimino) (MMI), 3′-N-carbamate, or morpholino carbamate.

In some aspects, the biologically active molecule (payload) is an antimir. As used herein, the terms “antimir,” “anti microRNA,” “anti miRNA,” and variants thereof refer to molecules (e.g., synthetically generated molecules) that are used to neutralize microRNA (miRNA) function in cells for desired responses. MiRNA are complementary sequences (approx. 20-22 bp) to mRNA that are involved in the cleavage of RNA or the suppression of the translation. By controlling the miRNA that regulate mRNAs in cells, antimirs (also called anti-miRNA oligonucleotides, AMOs, or antagomirs) can be used as further regulation as well as for therapeutic for certain cellular disorders. This regulation can occur through a steric blocking mechanism as well as hybridization to miRNA.

These interactions within the body between antimirs and a miRNA can be for therapeutics in disorders in which over/under expression occurs or aberrations in miRNA lead to coding issues. Some of the miRNA-linked disorders that are encountered in the humans include cancers, muscular diseases, autoimmune disorders, and viruses.

Various components of antimirs can be manipulated to affect the binding affinity and potency of the antimir. The 2′-sugar of the antimirs can be modified by introducing fluorine or various methyl groups, almost all with an increase in binding affinity. However, some of these modified 2′-sugar antimirs lead to negative effects on cell growth. Modifying the 5′-3′ phosphodiester backbone linkage to a phosphorothioate (P—S) backbone linkage is also known to have an effect on target affinity. Using the P—S mutation was shown to decrease the Tm of the oligonucleotide, which leads to a lower target affinity. A final requirement for antimirs is mismatch specificity and length restrictions. Due to miRNAs in the same families sharing “seed” (shared) sequences and differ by only a couple of additional nucleotides; one antimir can potentially target multiple miRNA sequences.

In some aspects, the payload comprises a therapeutic oligonucleotide or a combination thereof. In some aspects, the payload is a therapeutic oligonucleotide disclosed below.

In some aspects the therapeutic oligonucleotide is selected from the group consisting of 1018 ISS, AB-729, abetimus, AEG35156 (GEM640), afovirsen, aganirsen, agatolimod, alicaforsen, ALNAAT-02, amlivirsen, anivamersen, apatorsen, aprinocarsen, APTA-16, AR-177 (ZINTEVIR™), ARC19499 (BAX-499), archexin, AROANG-3, AROAPOC-3, ARO-HSD, AS1411 (AGRO100), ASM-8, asvasiran, atesidorsen, ATL-1102, ATU-027, avacincaptad pegol (ZIMURA™), AVI-4126 (Resten-MP™), AVI-7288, AVI-7537, AVT-02, AZD-8233, AZD-8701, baliforsen, bamosiran, bazlitoran, BC007, beclanorsen, belcesiran, bepirovirsen, bevasiranib, BIIB-080, BMN 044, BMN 053, brivoligide, casimersen, cavrotolimod, cemdisiran, cenersen, cepadacursen (CIVI 008), cimdelirsen, cobitolimod, cobomarsen, CODA-001 (NEXAGON™) cofirasersen, cosdosiran, CpG 7909, CPG-8954, cupabimod, custirsen, danvatirsen, daplusiran, defibrotide (DEFITELIO™), dematirsen, donidalorsen, drisapersen (KYNDRISA™), DYN-101, edifoligide, egaptivon pegol, EIF-4E, eluforsen, emapticap pegol, eplontersen, eteplirsen (EXONDYS 51™), fazisiran, fesomersen, fitusiran, fomivirsen (VITRAVENE™), frenlosirsen, gataparsen, givosiran (GIVLAARI™), GNKG-168 (CPG-685), golodirsen (SRP-4053, VYONDYS 53™), GPI-2A, GTI-2040 (LOR-2040), GTI-2501, GTX-102, HBVAXPRO, imetelstat, IMT-504, inclisiran, inotersen (TEGSEDI™), ION-224, ION-253, ION-363, ION-464, ION-541, ION-859, IONIS-AGTLRx, IONIS-APO(a)-Rx, IONISAR-2.5Rx, IONIS-C9Rx, IONIS-DNM2-2.5Rx, IONISENAC-2.5Rx, IONIS—FB-LRx, IONIS-FXILRx, IONIS-FXIRx, IONIS-GCGRRx, IONIS-HBVLRX, IONIS-MAPTRx, IONIS-PKKRx, IONISTMPRSS-6LRx, IONIS-TTRRx, ISIS EIF4E Rx, ISIS-104838, ISIS-1082, ISIS-113715, ISIS-2503, ISIS-333611, ISIS-426115, ISIS-449884, ISIS-463588, ISIS-5132, ISIS-702843, ISIS-757456, ISIS-863633, ISTH-0036, JNJ-3989, lademirsen, lexanersen (WVE-120102), lexaptepid pegol (NOX—H94), litenimod, LSP-GR3, lumasiran, mipomersen (KYNAMRO™), miravirsen, monarsen, mongersen, MT-5745, MTL-CEBPA, ND-L02-s0201 (BMS-986263), nedosiran, NS-089, nusinersen (SPINRAZA™) oblimersen (SPC2996, GENASENSE™), olaptesed pegol (NOX-A12), olezarsen, olpasiran, OLX-101, patisiran (ONPATTRO™), pegaptanib (MACUGEN™), PEGnivacogin, pegpleranib (FOVISTA™), pelacarsen, prexigebersen, PUL-042, QPI-1007, QR-1123, QRX-421a, radavirsen, remlarsen, renadirsen, revusiran, RG-012, RG-101, RG-6346, RGLS-4326, rimigorsen, rosomidnar, rovanersen (WVE-120101), sapablursen, SB010, sepofarsen, siG-12D-LODER, SLN124, SR-063, SRP-5051, STK-001, STP-705, suvodirsen, tadnersen, temavirsen, teprasiran, tilsotolimod, tivanisiran (SYLENTIS™), tofersen, tominersen, tomligisiran, TOP-1731, trabedersen (AP-12009), trecovirsen, varodarsen, VEGLIN 3, vidutolimod, viltolarsen (VILTEPSO™), VIR-2218, volanesorsen (WAYLIVRA™), vupanorsen, vutrisiran, WVE-003, WVE-004, WVEN-531, zilebesiran, and zilganersen.

In some aspects, the payload comprises an antisense oligonucleotide that targets an apoptosis regulator. In some aspects, the apoptosis regulator is pro-apoptotic gene product, e.g., FasL (Fas ligand), BAX, BID, BAK or BAD. In some aspects, the apoptosis regulator is an anti-apoptotic gene product, e.g, Bcl-XI, an IAP (e.g., XIAP), or Bcl-2. In some aspects, the apoptosis regulator is a prosurvival factor such as cFLIP, BNIP3, FADD, Akt, or NF-κB. In some aspects, the nucleic acid therapeutic agent is 1018 ISS, also known as ISS-1018, which is a CpG oligonucleotide that functions as an immune stimulant. The oligonucleotide sequence of 1018 ISS is SEQ ID NO: 142.

In some aspects, the nucleic acid therapeutic agent is AEG35156, an antisense oligonucleotide for the treatment of hepatocellular carcinoma, acute myeloid leukemia, B-cell lymphoma, chronic lymphocytic leukemia, multiple sclerosis, non-small cell lung cancer, or pancreatic cancer that targets X-Linked Inhibitor of Apoptosis (XIAP). The oligonucleotide sequence of AEG35156 is SEQ ID NO: 143. In some aspects, the nucleic acid therapeutic agent is AB-729, an anti-miRNA (antimir) for the treatment of hepatitis B infection that targets hepatitis virus B's HBsAg. In some aspects, the nucleic acid therapeutic agent is abetimus, an immunosuppressant oligonucleotide for the treatment of lupus nephritis. The oligonucleotide sequences of abetimus and their sequences are: subunit 1 (SEQ ID NO: 144), subunit 2 (SEQ ID NO: 145), subunit 3 (SEQ ID NO: 146), subunit 4 (SEQ ID NO: 147), subunit 5 (SEQ ID NO: 148), subunit 6 (SEQ ID NO: 149), subunit 7 (SEQ ID NO: 150), and subunit 8 (SEQ ID NO: 151). In some aspects, the nucleic acid therapeutic agent is afovirsen, an antisense oligonucleotide for the treatment of human papillomavirus infection that targets the mRNA of human papillomavirus. The oligonucleotide sequence of afovirsen is SEQ ID NO: 152. In some aspects, the nucleic acid therapeutic agent is aganirsen, also known as GS101, which is an antisense oligonucleotide used for the inhibition of corneal neovascularization, a major risk factor of corneal graft rejection that targets insulin receptor substrate-1 (IRS1). The oligonucleotide sequence of aganirsen is SEQ ID NO: 153. In some aspects, the nucleic acid therapeutic agent is agatolimod, also known as CPG-7909, AMA1-C1 or PF-3512676, which is a CpG oligodeoxynucleotide for the treatment of cancers such as basal cell cancer, non-Hodgkin's lymphoma, breast cancer, metastatic or recurrent malignancies, non-small cell lung cancer, infectious diseases, allergies, and asthma that acts as a toll-like receptor 9 (TLR9) agonist. The oligonucleotide sequence of agatolimod is SEQ ID NO: 154. In some aspects, the nucleic acid therapeutic agent is alicaforsen, an antisense oligonucleotide for the treatment of acute distress flares in moderate to severe inflammatory bowel disease that targets ICAM-1. The oligonucleotide sequence of alicaforsen is SEQ ID NO: 155. In some aspects, the nucleic acid therapeutic agent is AGRO100, also known as AS1411, which is an aptamer used for the treatment of acute myeloid leukemia, advanced solid tumors, metastatic renal cell carcinoma, and myeloid leukemia that targets IKBKG. The oligonucleotide sequence of AGRO100 is SEQ ID NO: 156. In some aspects, the nucleic acid therapeutic agent is amlivirsen, an antiviral antisense oligonucleotide. The oligonucleotide sequence of amlivirsen is SEQ ID NO: 157. In some aspects, the nucleic acid therapeutic agent is anivamersen and/or pegnivacogin. Anivamersen and pegnivacogin are components of the REG1 anticoagulation system. Pegnivacogin is an RNA aptamer inhibitor of coagulation factor IXa and anivamersen is a complementary sequence reversal oligonucleotide. The oligonucleotide sequence of pegnivacogin is SEQ ID NO: 158. The oligonucleotide sequence of anivamersen is SEQ ID NO: 159. In some aspects, the nucleic acid therapeutic agent is apatorsen, also known as OGX-427, which is an antisense oligonucleotide used for the treatment of advanced squamous cell lung cancers that targets Hsp27. The oligonucleotide sequence of apatorsen is SEQ ID NO: 160. In some aspects, the nucleic acid therapeutic agent is aprinocarsen, an antisense oligonucleotide for the treatment of cancer that targets PKC-α. The oligonucleotide sequence of aprinocarsen is SEQ ID NO: 161. In some aspects, the nucleic acid therapeutic agent is APTA-16, an aptamer for the treatment of acute myeloid leukemia, myelodysplastic syndromes, or liver cancer, that targets histone methyltransferase. In some aspects, the nucleic acid therapeutic agent is AR-177, also known as ZINTEVIR™, which is an oligonucleotide analogue that functions as an integrase inhibitor, and can be used for the treatment of HIV-1 infection. The oligonucleotide sequence of AR-177 is SEQ ID NO: 162. In some aspects, the nucleic acid therapeutic agent is ARC19499, also known as BAX-499, which is an RNA aptamer for the treatment of hemophilia that targets TFPI. The oligonucleotide sequence of ARC19499 is SEQ ID NO: 163. In some aspects, the nucleic acid therapeutic agent is archexin, also known as RX-201, which is an antisense oligonucleotide for the treatment of metastatic renal cancer, ovarian cancer, renal cell carcinoma, glioblastoma, stomach cancer, pancreatic cancer, lung cancer, or cervical carcinomas that targets the AKT-1 protein kinase. The oligonucleotide sequence of archexin is SEQ ID NO: 164. In some aspects, the nucleic acid therapeutic agent is asvasiran, a siRNA for the treatment of respiratory syncytial virus infection that targets the RSV N gene. The oligonucleotide sequence of asvasiran is a duplex RNA comprising the antisense sequence set forth in SEQ ID NO: 165 and the sense sequence set forth in SEQ ID NO: 166. In some aspects, the nucleic acid therapeutic agent is atesidorsen, also known as ATL 1103, which is an antisense oligonucleotide for the treatment of acromegaly, cancer, or diabetic retinopathy that targets somatotropin receptors. The oligonucleotide sequence of atesidorsen is SEQ ID NO: 167. In some aspects, the nucleic acid therapeutic agent is ATU-027. ATU-027 is a siRNA targeting protein kinase N3 that inhibits cancer progression, e.g., in prostate and pancreatic cancer. The oligonucleotide sequence of ATU-027 is a RNA duple comprising the antisense sequence set forth in SEQ ID NO: 168 and the sense sequence set forth in SEQ ID NO: 169. In some aspects, the nucleic acid therapeutic agent is AVT-02 developed by Avontec GmbH. AVT-02 is a short, double stranded oligonucleotide decoy for the treatment of psoriasis vulgaris that targets STAT-1. In some aspects, the nucleic acid therapeutic agent is avacincaptad pegol (ZIMURA™). Avacincaptad pegol is PEG-conjugated oligonucleotide for the treatment of polypoidal choroidal vasculopathy, Stargardt disease, or wet age-related macular degeneration that functions as a complement C5 inhibitor. The oligonucleotide sequence of avacincaptad pegol is SEQ ID NO: 170. In some aspects, the nucleic acid therapeutic agent is AVI-7537. AVI-7537 is a morpholino antisense oligonucleotide that targets the VP24 gene of Ebola virus. The oligonucleotide sequence of AVI-7537 is SEQ ID NO: 171. In some aspects, the nucleic acid therapeutic agent is AVI-7288. AVI-7288 is a morpholino antisense oligonucleotide that targets Marburg virus nucleoprotein (NP).

In some aspects, the nucleic acid therapeutic agent is baliforsen. Baliforsen, also known as IONIS-598769, is an antisense oligonucleotide for the treatment of myotonic dystrophy. The oligonucleotide sequence of baliforsen is SEQ ID NO: 172. In some aspects, the nucleic acid therapeutic agent is bamosiran. Bamosiran, also known as SYL-040012, is a siRNA for the treatment of glaucoma or ocular hypertension that targets beta 2 adrenergic receptors. The oligonucleotide sequence of bamosiran is a duplex RNA comprising an antisense strand having the sequence set forth in SEQ ID NO:173 and a sense strand having the sequence set forth in SEQ ID NO: 174. In some aspects, the nucleic acid therapeutic agent is bazlitoran. Bazlitoran, also known as IMO-8400, is a DNA oligonucleotide for the treatment of Waldenstrom's macroglobulinemia that targets toll-like receptors TLR7, TLR8 and TLR9. The oligonucleotide sequence of bazlitoran is SEQ ID NO: 175. In some aspects, the nucleic acid therapeutic agent is BC007. BC007 is a non-modified DNA aptamer out of a family of aptamers that bind to and lead to the neutralization of autoantibodies that are directed against G-protein-coupled receptors (GPCR-AABs). BC007 binds to 131-adrenergic-receptor-autoantibodies. BC007 can be used for the treatment of dilated cardiomyopathy or chronic fatigue syndrome. In some aspects, the nucleic acid therapeutic agent is beclanorsen. Beclanorsen, also known as SPC-2996, is an antisense oligonucleotide for the treatment of lymphoid leukemias that targets Bcl-2. The oligonucleotide sequence of beclanorsen is SEQ ID NO: 176. In some aspects, the nucleic acid therapeutic agent is bepirovirsen. Bepirovirsen, also known as ISIS-505358, ISIS-GSK3RX, GSK-3228836 or IONIS HBVRX, is an antisense oligonucleotide for the treatment of hepatitis B. The oligonucleotide sequence of bepirovirsen is SEQ ID NO: 177. In some aspects, the nucleic acid therapeutic agent is bevasiranib. Bevasiranib is a siRNA for the treatment of exudative age-related macular degeneration that targets VEGF. The oligonucleotide sequence of bevasiranib is an RNA duplex comprising an antisense strand having the sequence set forth in SEQ ID NO: 178 and a sense strand having the sequence set forth in SEQ ID NO: 179. In some aspects, the nucleic acid therapeutic agent is BMN 044. BMN 044, also known as PRO44, is an antisense oligonucleotide for the treatment of Duchenne muscular dystrophy that targets mRNA encoding dystrophin. In some aspects, the nucleic acid therapeutic agent is BMN 053. BMN 053, also known as PRO53, is an antisense oligonucleotide for the treatment of Duchenne muscular dystrophy that targets dystrophin. In some aspects, the nucleic acid therapeutic agent is brivoligide. Brivoligide is a 23 bp decoy DNA that functions an early growth response protein 1 inhibitor. Brivoligide can be used to treat pain, e.g., postoperative pain. The oligonucleotide sequence of brivoligide is a duplex DNA comprising SEQ ID NO: 180 and SEQ ID NO: 181.

In some aspects, the nucleic acid therapeutic agent is casimersen. Casimersen is a morpholino antisense oligonucleotide for the treatment of Duchenne muscular dystrophy that targets dystrophin's exon 45. The oligonucleotide sequence of casimersen is SEQ ID NO: 182. In some aspects, the nucleic acid therapeutic agent is cavrotolimod. Cavrotolimod is an immunostimulant oligonucleotide that functions as a TLR9 agonist, and can be used for the treatment of hematological malignancies, Merkel cell carcinoma, solid tumors, or squamous cell cancer. The oligonucleotide sequence of cavrotolimod is SEQ ID NO: 183. In some aspects, the nucleic acid therapeutic agent is cemdisiran. Cemdisiran, also known as AD062643, is a siRNA for the treatment of hemolytic uremic syndrome, IgA nephropathy, paroxysmal nocturnal hemoglobinuria, or myasthenia gravis that targets complement C5. The oligonucleotide sequence of cemdisiran is an RNA duplex comprising an antisense strand having the sequence set forth in SEQ ID NO: 184 and a sense strand having the sequence set forth in SEQ ID NO: 185. In some aspects, the nucleic acid therapeutic agent is cenersen. Cenersen is an antisense oligonucleotide for the treatment of myelodysplastic syndromes, acute myeloid leukemia, or chronic lymphocytic leukemia that targets p53. The oligonucleotide sequence of cenersen is SEQ ID NO: 186. In some aspects, the nucleic acid therapeutic agent is cobitolimod. Cobitolimod is an oligodeoxyribonucleotide for the treatment of ulcerative colitis or brain edema that is an agonist of Toll-like 9 receptors. The oligonucleotide sequence of cobitolimod is SEQ ID NO: 187. In some aspects, the nucleic acid therapeutic agent is cobomarsen. Cobomarsen, also known as MRG-106 or M11667, is an anti-miRNA (antimir) for the treatment of cutaneous T cell lymphoma, adult T cell leukemia-lymphoma, chronic lymphocytic leukemia, diffuse large B cell lymphoma, or amyotrophic lateral sclerosis that targets miR-155. In some aspects, the nucleic acid therapeutic agent is CODA-001. CODA-001, also known as NEXAGON™, is an antisense oligonucleotide for the treatment of wounds, leg ulcers, diabetic foot ulcers, or corneal injuries that targets gap junctions. NEXAGON™ is a natural, unmodified oligonucleotide (30-mer) that downregulates expression of the key gap junction protein Cx43. The oligonucleotide sequence of CODA-001 is SEQ ID NO: 188. In some aspects, the nucleic acid therapeutic agent is cofirasersen. Cofirasersen, also known as is an IONIS-ENACRX and ION-827359, is an antisense oligonucleotide for the treatment of pulmonary disease, chronic bronchitis, or cystic fibrosis that targets ENaC. The oligonucleotide sequence of cofirasersen is SEQ ID NO: 189. In some aspects, the nucleic acid therapeutic agent is cosdosiran. Cosdosiran, also known as QPI-1007, is a neuroprotective siRNA for the treatment of nonarteritic anterior ischemic optic neuropathy that inhibits caspase 2 synthesis. The oligonucleotide sequence of cosdosiran is an RNA duplex comprising an antisense strand having the sequence set forth in SEQ ID NO: 190 and a sense strand having the sequence set forth in SEQ ID NO: 191. In some aspects, the nucleic acid therapeutic agent is CPG-8954. CPG-8954 is a CpG oligonucleotide for the treatment of cancer and viral infections. The oligonucleotide sequence of CPG-8954 is SEQ ID NO: 192. In some aspects, the nucleic acid therapeutic agent is cupabimod. Cupabimod, also known as AMG-0103, is an oligonucleotide for the treatment of pain, e.g., chronic discogenic lumbar back pain. The oligonucleotide sequence of cupabimod is a double stranded DNA comprising the sequences SEQ ID NO: 193 and SEQ ID NO: 194. In some aspects, the nucleic acid therapeutic agent is custirsen. Custirsen, also known as OGX-011 and ISIS-112989, is an antisense oligonucleotide for the treatment of metastatic castrate resistant prostate cancer that targets clusterin. The oligonucleotide sequence of custirsen is SEQ ID NO: 195.

In some aspects, the nucleic acid therapeutic agent is danvatirsen. Danvatirsen, also known as AZD 9150 and ISIS-481464, is an antisense oligonucleotide for the treatment of bladder cancer, colorectal cancer, head and neck cancer, malignant ascites, non-small cell lung cancer, pancreatic cancer, solid tumors, liver cancer, non-Hodgkin's lymphoma, or diffuse large B cell lymphoma, and targets the STAT3 transcription factor. The oligonucleotide sequence of danvatirsen is SEQ ID NO: 196. In some aspects, the nucleic acid therapeutic agent is daplusiran. Daplusiran is an antiviral siRNA. The oligonucleotide sequence of daplusiran is an RNA duplex comprising an antisense strand having the sequence set forth in SEQ ID NO: 197 and a sense strand having the sequence set forth in SEQ ID NO: 198. In some aspects, the nucleic acid therapeutic agent is defibrotide (DEFITELIO™). Defibrotide, also known as DASOVAS™, NORAVID™, or PROCICLIDE™, is a heparanase inhibitor that functions as an angiogenesis and platelet aggregation inhibitor. Defibrotide is a mixture of single-stranded oligonucleotides that can be used for the treatment of veno-occlusive disorders, graft-versus-host disease, neurological disorders, thrombotic microangiopathies, deep vein thrombosis, thrombosis, diabetic nephropathies, or multiple myeloma. In some aspects, the nucleic acid therapeutic agent is the antisense oligonucleotide dematirsen. The oligonucleotide sequence of dematirsen is SEQ ID NO: 199. In some aspects, the nucleic acid therapeutic agent is the antisense oligonucleotide donidalorsen. Donidalorsen, also known as ISIS-721744, is a plasma kallikrein inhibitor that can be used for the treatment of COVID 2019 infections, hereditary angioedema, or acute respiratory disease. The oligonucleotide sequence of donidalorsen is SEQ ID NO: 200. In some aspects, the nucleic acid therapeutic agent is drisapersen (KYNDRISA™) Drisapersen, also known as GSK 2402968A, is an antisense oligonucleotide for the treatment of Duchenne muscular dystrophy that targets mRNA encoding dystrophin. The oligonucleotide sequence of drisapersen is SEQ ID NO: 201.

In some aspects, the nucleic acid therapeutic agent is edifoligide. Edifoligide is a 14 bp decoy DNA that functions as a CDC2 kinase inhibitor, and can be used for the treatment of coronary artery restenosis or vascular graft occlusion. The oligonucleotide sequence of edifoligide is a double stranded DNA comprising the sequences SEQ ID NO: 202 and SEQ ID NO: 203. In some aspects, the nucleic acid therapeutic agent is egaptivon pegol. Egaptivon pegol, also known as ARC1779, is an aptamer for the treatment of intracranial embolism, cerebral thromboembolism, carotid stenosis, von Willebrand disease, thrombotic thrombocytopenic purpura, thrombotic microangiopathy, thrombosis, or acute myocardial infarction that targets VWF GP1BA. The oligonucleotide sequence of egaptivon pegol is SEQ ID NO: 204. In some aspects, the nucleic acid therapeutic agent is EIF-4E ASO. EIF-4E ASO is an antisense oligonucleotide for the treatment of prostate cancer. The oligonucleotide sequence of EIF-4E ASO is SEQ ID NO: 205. In some aspects, the nucleic acid therapeutic agent is eluforsen. Eluforsen, also known as QR-010, is an oligonucleotide partly complementary to Phe508del-CFTR RNA. Eluforsen, also known as QR-010 is designed to repair CFTR-encoded mRNA. The oligonucleotide sequence of eluforsen is SEQ ID NO: 206. In some aspects, the nucleic acid therapeutic agent is emapticap pegol. Emapticap pegol, also known as NOX-E36, is an aptamer for the treatment of systemic lupus erythematosus type 2, diabetes mellitus, chronic inflammatory diseases, albuminuria, and renal impairment that targets CCL2. The oligonucleotide sequence of emapticap pegol is SEQ ID NO: 207. In some aspects, the nucleic acid therapeutic agent is eplontersen. Eplontersen, also known as ION-TTR-LRX or AKCEA-TTR-LRX, is an antisense oligonucleotide that functions as a prealbumin expression inhibitor, and can be used to treat amyloidosis or transthyretin-related hereditary amyloidosis. The oligonucleotide sequence of eplontersen is SEQ ID NO: 208. In some aspects, the nucleic acid therapeutic agent is eteplirsen (EXONDYS 51™). Eteplirsen, also known, AVI-4658, is an antisense oligonucleotide for the treatment of Duchenne muscular dystrophy that targets DMD exon 51. The oligonucleotide sequence of eteplirsen is SEQ ID NO: 209. In some aspects, the nucleic acid therapeutic agent is the antisense oligonucleotide fesomersen. The oligonucleotide sequence of fesomersen is SEQ ID NO: 210. In some aspects, the nucleic acid therapeutic agent is fitusiran. Fitusiran, also known as ALN-57213, is a siRNA for the treatment of hemophilia A and B that targets SERPINC1. The oligonucleotide sequence of fitusiran is a RNA duplex comprising an antisense strand having the sequence set forth in SEQ ID NO: 211 and a sense strand having the sequence set forth in SEQ ID NO: 212.

In some aspects, the nucleic acid therapeutic agent is fomivirsen (VITRAVENE™) Fomivirsen is an antisense oligonucleotide for the treatment of cytomegalovirus-induced retinitis and HIV infections that targets cytomegalovirus mRNA. The oligonucleotide sequence of fomivirsen is SEQ ID NO: 213. In some aspects, the nucleic acid therapeutic agent is gataparsen. Gataparsen is an antisense oligonucleotide for the treatment of acute myeloid leukemia, non-small cell lung cancer, or prostate cancer that targets BIRC5. The oligonucleotide sequence of gataparsen is SEQ ID NO: 214. In some aspects, the nucleic acid therapeutic agent is givosiran (GIVLAARI™). Givosiran is a siRNA for the treatment of acute hepatic porphyria that targets 5-aminolevulinate synthetase (ALAS1). The oligonucleotide sequence of givosiran is antisense SEQ ID NO: 215 and sense SEQ ID NO: 216. In some aspects, the nucleic acid therapeutic agent is GNKG-168. GNKG-168, also known as CPG-685, is an oligonucleotide that functions as a TLR9 agonist. GNKG-168 can be used for the treatment of chronic lymphocytic leukemia. The oligonucleotide sequence of GNKG-168 is SEQ ID NO: 217. In some aspects, the nucleic acid therapeutic agent is golodirsen (VYONDYS 53™). Golodirsen, also known as SRP-4053 and VYONDYS 53™, is an antisense oligonucleotide used to treat Duchenne muscular dystrophy via splicing modulation that targets DMD exon 53. The oligonucleotide sequence of golodirsen is SEQ ID NO: 218. In some aspects, the nucleic acid therapeutic agent is GPI-2A. GPI-2A is an antisense oligonucleotide for the treatment of HIV that inhibits the expression of human immunodeficiency virus type 1 capsid. The oligonucleotide sequence of GPI-2A is SEQ ID NO: 219.

In some aspects, the nucleic acid therapeutic agent is GTI-2040. GTI-2040, also known as LOR-2040, is an antisense oligonucleotide for the treatment of renal cell carcinoma that functions as a DNA synthesis inhibitor. GTI-2040 can also be used for the treatment of acute myeloid leukemia, bladder cancer, breast cancer, chronic myeloid leukemia, colorectal cancer, myelodysplastic syndromes, non-small cell lung cancer, or prostate cancer. The oligonucleotide sequence of GTI-2040 is SEQ ID NO: 220. In some aspects, the nucleic acid therapeutic agent is GTI-2501. GTI-2501 is an antisense oligonucleotide for the treatment of renal cell carcinoma that functions as a DNA synthesis inhibitor by targeting the ribonucleoside-diphosphate reductase large subunit. GTI-2501 can also be used for the treatment of acute myeloid leukemia, bladder cancer, breast cancer, chronic myeloid leukemia, colorectal cancer, myelodysplastic syndromes, non-small cell lung cancer, or prostate cancer. The oligonucleotide sequence of GTI-2501 is SEQ ID NO: 221.

In some aspects, the nucleic acid therapeutic agent is HBVAXPRO. HBVAXPRO is a decoy for the treatment of Hepatitis B that targets HBV. In some aspects, the nucleic acid therapeutic agent is IMT-504. IMT-504 is a B cell immunostimulant oligonucleotide for the treatment of diabetes mellitus, rabies, breast cancer, chronic lymphocytic leukemia, hepatitis B, influenza virus infections, neuropathic pain, osteoporosis, or sepsis. The oligonucleotide sequence of IMT-504 is SEQ ID NO: 222. In some aspects, the nucleic acid therapeutic agent is inclisiran. Inclisiran, also known as ALN-60212, is a siRNA for the treatment of hypercholesterolemia that targets PCSK9. The oligonucleotide sequence of inclisiran is an RNA duplex comprising an antisense strand having the sequence set forth in SEQ ID NO: 223 and a sense strand having the sequence set forth in SEQ ID NO: 224. In some aspects, the nucleic acid therapeutic agent is inotersen (TEGSEDI™). Inotersen is an antisense oligonucleotide for the treatment of hereditary transthyretin-mediated amyloidosis or polyneuropathy that targets TTR. The oligonucleotide sequence of inotersen is SEQ ID NO: 225. In some aspects, the nucleic acid therapeutic agent is imetelstat. Imetelstat is a telomerase inhibitor oligonucleotide for the treatment of myelodysplastic syndromes, myelofibrosis, multiple myeloma, acute myeloid leukemia, chronic myeloid leukemia, breast cancer, essential thrombocythemia, lymphoproliferative disorders, non-small cell lung cancer, polycythemia vera, or solid tumors. The oligonucleotide sequence of imetelstat is SEQ ID NO: 226.

In some aspects, the nucleic acid therapeutic agent is IONIS-APO(a)-Rx. IONIS-APO(a)-Rx is an antisense oligonucleotide for the treatment of high lipoprotein levels that targets apolipoprotein A. In some aspects, the nucleic acid therapeutic agent is IONIS-C9Rx. IONIS-C9Rx is an antisense oligonucleotide for the treatment of ALS that targets C90RF72. In some aspects, the nucleic acid therapeutic agent is IONIS-DNM2-2.5Rx. IONIS-DNM2-2.5Rx is antisense oligonucleotide for the treatment of centronuclear myopathy that targets DNM2. In some aspects, the nucleic acid therapeutic agent is IONIS-FXIRx. IONIS-FXIRx is an antisense oligonucleotide for the treatment of total knee arthroplasty that targets Factor XI. In some aspects, the nucleic acid therapeutic agent is IONIS-GCGRRx. IONIS-GCGRRx is an antisense oligonucleotide for the treatment of type 2 diabetes that targets glucagon receptor. In some aspects, the nucleic acid therapeutic agent is IONIS-MAPTRx. IONIS-MAPTRx is an antisense oligonucleotide for the treatment of Alzheimer disease that targets MAPT. In some aspects, the nucleic acid therapeutic agent is IONIS-TTRRx. IONIS-TTRRx is an antisense oligonucleotide for the treatment of familial amyloid polyneuropathy (FAP) that targets transthyretin. In some aspects, the nucleic acid therapeutic agent is ISIS EIF4E Rx. ISIS EIF4E Rx is an antisense oligonucleotide for the treatment of castrate-resistant prostate cancer that targets eIF-4E. In some aspects, the nucleic acid therapeutic agent is ISIS-104838, ISIS-1082, ISIS-2503, ISIS-333611, ISIS-113715, ISIS-426115, ISIS-449884, ISIS-463588, ISIS-5132, ISIS-702843, or ISIS-757456.

In some aspects, the nucleic acid therapeutic agent is lademirsen. Lademirsen is an antisense oligonucleotide that targets miR-21. The oligonucleotide sequence of lademirsen is SEQ ID NO: 227. In some aspects, the nucleic acid therapeutic agent is lexaptepid pegol. Lexaptepid pegol, also known as NOX—H94, is an aptamer for the treatment of anemia, end stage renal disease, anemia of chronic disease, chronic diseases, or inflammation that targets hepcidin. The oligonucleotide sequence of lexaptepid pegol is SEQ ID NO: 228. In some aspects, the nucleic acid therapeutic agent is litenimod. Litenimod is a 26-mer modified oligodeoxynucleotides (ODN) that functions as a TLR9 agonist. The oligonucleotide sequence of litenimod is SEQ ID NO: 229. In some aspects, the nucleic acid therapeutic agent is lumasiran. Lumasiran is a siRNA for the treatment of primary hyperoxaluria type 1 that targets HAO1. The oligonucleotide sequence of lumasiran (OXLUMO™) is a double stranded RNA (dsRNA) comprising an antisense strand having the sequence set forth in SEQ ID NO: 230 and a sense strand having the sequence set forth in SEQ ID NO: 231. In some aspects, the nucleic acid therapeutic agent is mipomersen (KYNAMRO™). Mipomersen is antisense oligonucleotide for the treatment of familial hypercholesterolemia that targets APOB. The oligonucleotide sequence of mipomersen is SEQ ID NO: 232. In some aspects, the nucleic acid therapeutic agent is miravirsen. Miravirsen, also known as SPC3649, is an antisense oligonucleotide for the treatment of chronic hepatitis C (CHC) infection that targets miRNA-122. The oligonucleotide sequence of miravirsen is SEQ ID NO: 233. In some aspects, the nucleic acid therapeutic agent is mongersen. Mongersen, also known as GED-0301, is an antisense oligonucleotide for the treatment of Crohn's disease that targets SMAD7. The oligonucleotide sequence of mongersen is SEQ ID NO: 234.

In some aspects, the nucleic acid therapeutic agent is MTL-CEBPA. MTL-CEBPA is a small activating RNA (saRNA) designed to reduce immune suppression of myeloid cells by restoring C/EBP-α protein to normal levels using the RNA activation mechanism. MTL-CEBPA can be used for the treatment of liver cancer, solid tumors, colorectal cancer, hepatocellular carcinoma, or liver disorders. In some aspects, the nucleic acid therapeutic agent is ND-L02-s0201. ND-L02-s0201, also known as BMS-986263, is a siRNA for the treatment of extensive hepatic fibrosis that targets HSP47. In some aspects, the nucleic acid therapeutic agent is nedosiran. Nedosiran is a siRNA for the treatment of primary hyperoxaluria that targets LDHA. The oligonucleotide sequence of nedosiran is a double stranded RNA (dsRNA) comprising an antisense strand having the sequence set forth in SEQ ID NO: 235 and a sense strand having the sequence set forth in SEQ ID NO: 236. In some aspects, the nucleic acid therapeutic agent is nusinersen (SPINRAZA™). Nusinersen is an antisense oligonucleotide (splice modulator) for the treatment of infantile-onset spinal muscular atrophy that targets exon 7 of the Survival of Motor Neuron 2 (SMN2) splicing modulator. The oligonucleotide sequence of nusinersen is SEQ ID NO: 237. In some aspects, the nucleic acid therapeutic agent is oblimersen (GENASENSE™). Oblimersen is an antisense oligonucleotide for the treatment of melanoma that targets Bcl-2. The oligonucleotide sequence of oblimersen is SEQ ID NO: 238. In some aspects, the nucleic acid therapeutic agent is olaptesed pegol. Olaptesed pegol, also known as NOX-A12, is an aptamer for the treatment of chronic lymphocytic leukemia, multiple myeloma, hematopoietic stem cell transplantation, autologous stem cell transplantation, glioblastoma, metastatic colorectal cancer, or metastatic pancreatic cancer that targets CXCL12. The oligonucleotide sequence of olaptesed pegol is SEQ ID NO: 239. In some aspects, the nucleic acid therapeutic agent is olpasiran. Olpasiran is a siRNA for the treatment of cardiovascular disorders that targets lipoprotein(a) (Lp(a)). The oligonucleotide sequence of olpasiran is a double stranded RNA (dsRNA) comprising an antisense strand having the sequence set forth in SEQ ID NO: 240 and a sense strand having the sequence set forth in SEQ ID NO: 241.

In some aspects, the nucleic acid therapeutic agent is patisiran (ONPATTRO™) Patisiran is a siRNA for the treatment of hereditary transthyretin-mediated amyloidosis and neuropathy that targets transthyretin. The oligonucleotide sequence of patisiran is a double stranded RNA (dsRNA) comprising an antisense strand having the sequence set forth in SEQ ID NO: 242 and a sense strand having the sequence set forth in SEQ ID NO: 243. In some aspects, the nucleic acid therapeutic agent is pegaptanib (MACUGEN™). Pegaptanib is an aptamer for the treatment of wet macular degeneration neovascular age-related macular degeneration that targets VEGF. The oligonucleotide sequence of pegaptanib is SEQ ID NO: 244. In some aspects, the nucleic acid therapeutic agent is pegpleranib (FOVISTA™). Pegpleranib is an aptamer for the treatment of subfoveal neovascular age-related macular degeneration that targets PDGF-B. The oligonucleotide sequence of pegpleranib is SEQ ID NO: 245. In some aspects, the nucleic acid therapeutic agent is pelacarsen. Pelacarsen, also known as IONIC-APO(a)-LRX and ISIS-681257, is an antisense oligonucleotide form the treatment of hyperlipoproteinemia that targets apolipoprotein A. The oligonucleotide sequence of pelacarsen is SEQ ID NO: 246. In some aspects, the nucleic acid therapeutic agent is prexigebersen. Prexigebersen is an antisense oligonucleotide for the treatment of acute myeloid leukemia, myelodysplastic syndromes, chronic myeloid leukemia, precursor cell lymphoblastic leukemia-lymphoma, colorectal cancer, head and neck cancer, lymphoma, solid tumors, thyroid cancer, or breast cancer that targets GRB2. The oligonucleotide sequence of prexigebersen is SEQ ID NO: 247.

In some aspects, the nucleic acid therapeutic agent is radavirsen. Radavirsen, also known as AVI-7100, is an antisense oligonucleotide for the treatment of influenza A virus infections. The oligonucleotide sequence of radavirsen is SEQ ID NO: 248. In some aspects, the nucleic acid therapeutic agent is remlarsen. Remlarsen is a miRNA mimic for the treatment of cutaneous fibrosis. Remlarsen mimics miR-29. The oligonucleotide sequence of remlarsen is a double stranded RNA (dsRNA) comprising an antisense strand having the sequence set forth in SEQ ID NO: 249 and a sense strand having the sequence set forth in SEQ ID NO: 250. In some aspects, the nucleic acid therapeutic agent is renadirsen. Renadirsen, also known as renapersen, is an antisense oligonucleotide for the treatment of Duchenne muscular dystrophy that functions by stimulating the expression of dystrophin. The oligonucleotide sequence of renadirsen is SEQ ID NO: 251. In some aspects, the nucleic acid therapeutic agent is RESTEN-MP™. RESTEN-MP™, also known as AVI-4126, is an antisense oligonucleotide for the treatment of de novo native coronary artery lesions that targets c-myc. The oligonucleotide sequence of AVI-4126 is SEQ ID NO: 252. In some aspects, the nucleic acid therapeutic agent is revusiran. Revusiran, also known as AD-51547, is a siRNA for the treatment of hereditary amyloidosis that targets TTR. The oligonucleotide sequence of revusiran is a double stranded RNA (dsRNA) comprising an antisense strand having the sequence set forth in SEQ ID NO: 253 and a sense strand having the sequence set forth in SEQ ID NO: 254. In some aspects, the nucleic acid therapeutic agent is RGLS-4326. RGLS-4326 is antisense oligonucleotide antimir for the treatment of autosomal dominant polycystic kidney disease that targets miR-17. The oligonucleotide sequence of RGLS-4326 is SEQ ID NO: 255. In some aspects, the nucleic acid therapeutic agent is rimigorsen. Rimigorsen is an antisense oligonucleotide for the treatment of Duchenne muscular dystrophy that promotes the synthesis of functional dystrophin. The oligonucleotide sequence of rimigorsen is SEQ ID NO: 256. In some aspects, the nucleic acid therapeutic agent is rosomidnar. Rosomidnar, also known as PNT-100, is an oligonucleotide inhibitor of apoptosis regulator Bcl2 that can be used for the treatment of diffuse large B cell lymphoma, Richter's syndrome, non-Hodgkin's lymphoma, or solid tumors. The oligonucleotide sequence of rosomidnar is SEQ ID NO: 257.

In some aspects, the nucleic acid therapeutic agent is SB010. SB010 is an antisense oligonucleotide for the treatment of mild allergic asthma that targets GATA-3. In some aspects, the nucleic acid therapeutic agent is SLN124. SLN124 is a siRNA for the treatment of β-thalassemia that targets TMPRSS6. In some aspects, the nucleic acid therapeutic agent is SRP-5051. SRP-5051 is a PPMO antisense oligonucleotide for the treatment of Duchenne muscular dystrophy that target DMD exon 51. SRP-5051 is next generation eteplirsen, in that it targets the same population, those amenable to exon 51 skipping, but the compound is “charged”, meaning that its cell-penetrating capacity is increased. In some aspects, the nucleic acid therapeutic agent is RG-012. RG-012 is an antisense oligonucleotide antimir for the treatment of Aport syndrome that target miR-21. In some aspects, the nucleic acid therapeutic agent is suvodirsen. Suvodirsen, also known as WVE-210201, is an antisense oligonucleotide for the treatment of Duchenne muscular dystrophy that targets DMD exon 51. The oligonucleotide sequence of suvodirsen is SEQ ID NO: 258.

In some aspects, the nucleic acid therapeutic agent is temavirsen. Temavirsen, also known as RG-101 and RG-2459, is an antiviral antisense oligonucleotide that targets the hepatitis C virus. The oligonucleotide sequence of temavirsen is SEQ ID NO: 259. In some aspects, the nucleic acid therapeutic agent is teprasiran. Teprasiran, also known as QPI-1002, is a siRNA inhibitor of tumor suppressor protein p53. Teprasiran can be used for the treatment of acute kidney injury or delayed graft function. The oligonucleotide sequence of teprasiran is a double stranded RNA (dsRNA) comprising an antisense strand having the sequence set forth in SEQ ID NO: 260 and a sense strand having the sequence set forth in SEQ ID NO: 261. In some aspects, the nucleic acid therapeutic agent is tivanisiran (SYLENTIS™). Tivanisiran, also known as SYL-1001, is a siRNA that targets the Transient Receptor Potential Vanilloid-1 (TRPV1) channel family. Tivanisiran can be used to treat ocular pain. The oligonucleotide sequence of tivanisiran is a double stranded RNA (dsRNA) comprising an antisense strand having the sequence set forth in SEQ ID NO: 262 and a sense strand having the sequence set forth in SEQ ID NO: 263. In some aspects, the nucleic acid therapeutic agent is tofersen. Tofersen, also known as IONIS-SOD1Rx and BIIB-067, is an antisense oligonucleotide for the treatment of Amyotrophic Lateral Sclerosis (ALS) that targets SOD1. The oligonucleotide sequence of tofersen is SEQ ID NO: 264. In some aspects, the nucleic acid therapeutic agent is tominersen. Tominersen, also known as IONIS-HTTRx, RG-6042 and ISIS-443139, is an antisense oligonucleotide for the treatment of Huntington's disease that targets HTT. The oligonucleotide sequence of tominersen is SEQ ID NO: 265. In some aspects, the nucleic acid therapeutic agent is trabedersen. Trabedersen, also known as AP-12009 and A-12009, is an antisense oligonucleotide that is an inhibitor of transforming growth factor beta 2. Trabedersen can be used to treat glioblastoma, malignant melanoma, pancreatic cancer, COVID 2019 infections, COVID-19 pneumonia, ovarian cancer, colorectal cancer, or anaplastic astrocytoma. The oligonucleotide sequence of trabedersen is SEQ ID NO: 266. In some aspects, the nucleic acid therapeutic agent is trecovirsen. Trecovirsen is an antisense oligonucleotide for the treatment of AIDS that targets GAG. The oligonucleotide sequence of trecovirsen is SEQ ID NO: 267.

In some aspects, the nucleic acid therapeutic is vidutolimod. Vidutolimod, also known as CMP-001 is an immunostimulant oligonucleotide for the treatment of malignant melanoma, head and neck cancer, lymphoma, solid tumors, squamous cell cancer, colorectal cancer, non-small cell lung cancer, allergic asthma, atopic dermatitis, hepatitis B, perennial allergic rhinitis, or seasonal allergic rhinitis. The oligonucleotide sequence of vidutolimod is SEQ ID NO: 268. In some aspects, the nucleic acid therapeutic agent is viltolarsen (VILTEPSO™), SEQ ID NO: 269. Viltolarsen is an antisense oligonucleotide for the treatment of Duchenne's muscular dystrophy that targets DMD exon 53. In some aspects, the nucleic acid therapeutic agent is volanesorsen (WAYLIVRA™), SEQ ID NO: 270. Volanesorsen, also known as ISIS-304801, is an antisense oligonucleotide for the treatment of hypertriglyceridemia, familial chylomicronemia syndrome, or familial partial lipodystrophy that targets ApoC-III. In some aspects, the nucleic acid therapeutic agent is vupanorsen, SEQ ID NO: 271. Vupanorsen, also known as IONIS-ANGPTL3-LRx, AKCEA-ANGPTL3-LRx and ISIS-703802, is an antisense oligonucleotide conjugate (GalNAc3) for the treatment of cardiovascular disease and reduce triglyceride and cholesterol levels, that targets angiopoietin-like 3 (ANGPTL3). In some aspects, the nucleic acid therapeutic agent is vutrisiran. Vutrisiran, also known as ALN-TTRsc02 and ALN-65492, is a siRNA for the treatment of hereditary amyloidosis that targets TTR. The oligonucleotide sequence of vutrisiran is a double stranded RNA (dsRNA) comprising an antisense strand having the sequence set forth in SEQ ID NO: 272 and a sense strand having the sequence set forth in SEQ ID NO: 273.

In some aspects, the nucleic acid therapeutic agent is WVE-120101 (rovanersen, also known as WV-1092) or WVE-120102 (lexanersen, also known as WV-2603). WVE-120101 and WVE-120102 are antisense oligonucleotides for the treatment of Huntington's disease that target mutant HTT. WVE-120101 and WVE-120102 interfere with the mutant mRNA copy of the HTT gene. The oligonucleotide sequence of WVE-120101 (rovanersen) is SEQ ID NO: 274. The oligonucleotide sequence of WVE-120102 (lexanersen) is SEQ ID NO: 275. In some aspects, the nucleic acid therapeutic agent is cepadacursen, SEQ ID NO: 276. In some aspects, cepadacursen can be used to treat diseases or conditions caused by abnormal expression levels and/or activity of PCSK9 selected from the group consisting of atherosclerosis, hypercholesterolemia (e.g., familiar hypercholesterolemia or statin resistant hypercholesterolemia), HDL/LDL cholesterol imbalance, dyslipidemia (e.g., familial hyperlipidemia (FCHL) or acquired hyperlipidemia), coronary artery disease (CAD), and coronary heart disease (CHD). In some aspects, the nucleic acid therapeutic agent is ISIS-863633, which has the sequence set forth in SEQ ID NO: 277. In some aspects, the nucleic acid therapeutic agent is ALNAAT-02, which targets SERPINA1. In some aspects, the nucleic acid therapeutic agent is AROANG-3, which targets ANGPTL3. In some aspects, the nucleic acid therapeutic agent is AROAPOC-3, which targets APOC3. In some aspects, the nucleic acid therapeutic agent is ARO-HSD, which targets HSD17B13. In some aspects, the nucleic acid therapeutic agent is AS1411, which targets nucleolin and has the sequence set forth in SEQ ID NO: 278. In some aspects, the nucleic acid therapeutic agent is ASM-8, which targets CCR4 and CSF2RB. In some aspects, the nucleic acid therapeutic agent is ATL-1102, which targets ITGA4. In some aspects, the nucleic acid therapeutic agent is AZD-8233, which targets PCSK9. In some aspects, the nucleic acid therapeutic agent is AZD-8701, which targets FOXP3. In some aspects, the nucleic acid therapeutic agent is belcesiran, which targets SERPINA1 and has the antisense sequence set forth in SEQ ID NO: 279 and the sense sequence set forth in SEQ ID NO: 280. In some aspects, the nucleic acid therapeutic agent is BIIB-080, which targets MAPT. In some aspects, the nucleic acid therapeutic agent is cimderlirsen, which targets GHR and has the sequence set forth in SEQ ID NO: 281. In some aspects, the nucleic acid therapeutic agent is CpG 7909, which targets TLR9. In some aspects, the nucleic acid therapeutic agent is DYN-101, which targets DYN2. In some aspects, the nucleic acid therapeutic agent is fazisiran, which targets SERPINA1. In some aspects, the nucleic acid therapeutic agent is frenlosirsen, which targets IRF4 and has the sequence set forth in SEQ ID NO: 282. In some aspects, the nucleic acid therapeutic agent is GTX-102, which targets UBE2A. In some aspects, the nucleic acid therapeutic agent is ION-224, which targets DGAT2. In some aspects, the nucleic acid therapeutic agent is ION-253. In some aspects, the nucleic acid therapeutic agent is ION-363, which targets FUS. In some aspects, the nucleic acid therapeutic agent is ION-464, which targets SNCA. In some aspects, the nucleic acid therapeutic agent is ION-541, which targets ATXN2. In some aspects, the nucleic acid therapeutic agent is ION-859, which targets LRRK2. In some aspects, the nucleic acid therapeutic agent is IONIS-AGTLRx, which targets AGT. In some aspects, the nucleic acid therapeutic agent is IONISAR-2.5Rx, which targets AR. In some aspects, the nucleic acid therapeutic agent is IONISENAC-2.5Rx, which targets SCNN1A. In some aspects, the nucleic acid therapeutic agent is IONIS—FB-LRx, which targets CFB. In some aspects, the nucleic acid therapeutic agent is IONIS-FXILRx, which targets F11. In some aspects, the nucleic acid therapeutic agent is IONIS-HBVLRX, which targets HBV. In some aspects, the nucleic acid therapeutic agent is IONIS-PKKRx, which targets KLKB1. In some aspects, the nucleic acid therapeutic agent is IONISTMPRSS-6LRx, which targets TMPRSS6. In some aspects, the nucleic acid therapeutic agent is ISTH-0036, which targets TGFB2. In some aspects, the nucleic acid therapeutic agent is JNJ-3989, which targets HBV. In some aspects, the nucleic acid therapeutic agent is LSP-GR3, which targets GR3 and has the sequence set forth in SEQ ID NO: 283. In some aspects, the nucleic acid therapeutic agent is monarsen, which targets AchE and has the sequence set forth in SEQ ID NO: 284. In some aspects, the nucleic acid therapeutic agent is MT-5745, which targets CHST15. In some aspects, the nucleic acid therapeutic agent is NS-089, which targets DMD. In some aspects, the nucleic acid therapeutic agent is olezarsen, which targets APOC3 and has the sequence set forth in SEQ ID NO: 285. In some aspects, the nucleic acid therapeutic agent is OLX-101, which targets CTGF. In some aspects, the nucleic acid therapeutic agent is PUL-042, which targets TLR2, TLR6, and TLR9 and has the sequence set forth in SEQ ID NO: 286. In some aspects, the nucleic acid therapeutic agent is QPI-1007, which targets CASP2. In some aspects, the nucleic acid therapeutic agent is QR-1123, which targets RHO. In some aspects, the nucleic acid therapeutic agent is QRX-421a, which targets USH2A. In some aspects, the nucleic acid therapeutic agent is RG-101, which targets miR-122. In some aspects, the nucleic acid therapeutic agent is RG-6346, which targets HBsAg. In some aspects, the nucleic acid therapeutic agent is sapablursen, which targets TMPRSS6 and has the sequence set forth in SEQ ID NO: 287. In some aspects, the nucleic acid therapeutic agent is sepofarsen, which targets CEP290 and has the sequence set forth in SEQ ID NO: 288. In some aspects, the nucleic acid therapeutic agent is siG-12D-LODER, which targets KRAS. In some aspects, the nucleic acid therapeutic agent is SR-063, which targets AR. In some aspects, the nucleic acid therapeutic agent is STK-001, which targets SVN1A. In some aspects, the nucleic acid therapeutic agent is STP-705, which targets PTGS2/TGFB1. In some aspects, the nucleic acid therapeutic agent is tadnersen, which targets C9orf72 and has the sequence set forth in SEQ ID NO: 289. In some aspects, the nucleic acid therapeutic agent is tilsotolimod, which targets TLR9 and has the set forth in SEQ ID NO: 290 and the sequence set forth in SEQ ID NO: 291. In some aspects, the nucleic acid therapeutic agent is tomligisiran, which has the antisense sequence set forth in SEQ ID NO: 292 and the sequence set forth in SEQ ID NO: 293. In some aspects, the nucleic acid therapeutic agent is TOP-1731, which has the sequence set forth in SEQ ID NO: 294. In some aspects, the nucleic acid therapeutic agent is varodarsen, which has the sequence set forth in SEQ ID NO: 295. In some aspects, the nucleic acid therapeutic agent is VEGLIN 3, which targets VEGF and has the sequence set forth in SEQ ID NO: 296. In some aspects, the nucleic acid therapeutic agent is VIR-2218, which targets HBsAg. In some aspects, the nucleic acid therapeutic agent is WVE-003, which targets HTT. In some aspects, the nucleic acid therapeutic agent is WVE-004, which targets C9orf72. In some aspects, the nucleic acid therapeutic agent is WVEN-531, which targets DMD. In some aspects, the nucleic acid therapeutic agent is zilebesiran, which targets AGT and has the antisense sequence set forth in SEQ ID NO: 297 and the sense sequence set forth in SEQ ID NO: 298. In some aspects, the nucleic acid therapeutic agent is zilganersen, which targets GFAP.

### I.B.vi Payload: Chemotherapy Agents

In some aspects, the payload comprises a chemotherapy agent. In some aspects, the chemotherapy agent comprises an alkylating agent, antimetabolite, anti-microtubule, topoisomerase inhibitor, cytotoxic antibiotic, or a combination thereof. In some aspects, the chemotherapy agent comprises, e.g., cyclophosphamide, methotrexate, 5-fluorouracil, doxorubicin, docetaxel, vinblastine, vincristine, prednisolone, bleomycin, etoposide, cisplatin, epirubicin, capecitabine, ifosfamide, folinic acid, oxaliplatin, vinorelbine, procarbazine, mistune, dacarbazine, or a combination thereof. In some aspects, the payload comprises a nucleoside analog selected from the group consisting of azacitidine, capecitabine, carmofur, cladribine, clofarabine, cytarabine, decitabine, floxuridine, fludarabine, fluorouracil, gemcitabine, mercaptopurine, nelarabine, pentostatin, tegafur, and tioguanine. In some aspects the payload comprises an antifolate selected from the group consisting of methotrexate, pemetrexed, and raltitrexed. In some aspects, the payload comprises a topoisomerase I inhibitor such as irinotecan or topotecan. In some aspects, the payload comprises an anthracycline such as daunorubicin, doxorubicin, epirubicin, idarubicin, mitoxantrone or valrubicin. In some aspects, the payload comprises a podophyllotoxin such as etoposide or teniposide. In some aspects, the payload comprises a taxane such a cabazitaxel, docetaxel, or paclitaxel. In some aspects, the payload comprises a vinca alkaloid selected from the group consisting of vinblastine, vincristine, vindesine, vinflunine, and vinorelbine. In some aspects, the payload comprises an alkylating agent selected from the group consisting of bendamustine, busulfan, carmustine, chlorambucil, chlormethine, cyclophosphamide, dacarbazine, fotemustine, ifosfamide, lomustine, melphalan, streptozotocin, and temozolomide. In some aspects, the payload comprises a platinum compound selected from the group consisting of carboplatin, cisplatin, nedaplatin, and oxaliplatin.

In some aspects, the payload comprises a targeted antineoplastic therapeutic agent. In some aspects, the targeted antineoplastic therapeutic agent comprises an antibody, e.g., a monoclonal antibody, or an antigen-binding portion thereof. In some aspects, the monoclonal antibody comprises alemtuzumab (anti-CD52), bevacizumab (anti-VEGF), cetuximab (anti-EGFR), denosumab (anti-RNAKL), gemtuzumab ozogamicin (anti-CD33), ibritumomab tiuxetan (anti-CD20), ipilimumab (anti-CTLA4), nivolumab (anti-PD1), ofatumumab (anti-CD20), panitumumab (anti-EGFR), pembrolizumab (anti-PD1), pertuzumab (anti-HER2), rituximab (anti-CD20), tositumomab (anti-CD20), trastuzumab (anti-HER2) or an antigen binding portion thereof.

In some aspects, the payload comprises a small molecules tyrosine kinase inhibitor. In some aspects, the tyrosine kinase inhibitor is selected from the group consisting of afatinib (EGFR, HER2 and HER4 inhibitor), aflibercept (VEGF and PGF inhibitor), axitinib (multikinase inhibitor), bosutinib (Bcr-Abl and SRc kinase inhibitor), crizotinib (ALK, HGFR, and RON inhibitor), dasatinib (BCR-ABL, SRC family, c-Kit, EPHA2 and PDGFR-β kinase inhibitor), erlotinib (EGFR inhibitor), gefitinib (EGFR inhibitor), imatinib (Bcr-Abl kinase inhibitor), lapatinib (HER2 inhibitor), nilotinib (Bcr-Abl kinase inhibitor), pazopanib (Multikinase inhibitor, including c-KIT, FGFR, PDGFR and VEGFR), ponatinib (Multikinase inhibitor (BEGFR, PDGFR, FGFR, EPH receptors and SRC families of kinases, and KIT, RET, TIE2 and FLT3), that also inhibits T135I Bcr-Abl kinase), regorafenib (Multikinase inhibitor for RET, VEGFR1, VEGFR2, VEGFR3, KIT, PDGFR-alpha, PDGFR-beta, FGFR1, FGFR2, TIE2, DDR2, Trk2A, Eph2A, RAF-1, BRAF, BRAFV600E, SAPK2, PTK5, and Bcr-Abl), ruxolitinib (JAK1 and JAK2 inhibitor), sorafenib (Multikinase inhibitor, including VEGF and PDGF receptor kinases), sunitinib (Multikinase inhibitor, including VEGF & PDGF receptor tyrosine kinases), and vandelanic (Tyrosine kinase inhibitor (TKI) with selective activity against RET, VEGFR-2 and EGFR). In some aspects, the payload comprises an mTOR inhibitor such as everolimus or temsirolimus. In some aspects, the payload comprises a retinoid selected from the group consisting of bexarotene (RXR agonist), isotretinoin (RXR and RAR agonist), tamibarotene (RAR agonist), and tretinoin (RXR and RAR agonist). In some aspects, the payload comprises an immunomodulatory agent (IMID) such as lenalidomide, thalidomide, or pomalidomide. In some aspects, the payload comprises a histone deacetylase inhibitor such as romidepsin, valproate, or vorinostat.

### I.B.vii Payload: AAV Vectors

In some aspects, the payload is inclusive of an AAV vector including linear or plasmid, circular vectors that encode a gene of interest (GOI). In related aspects, the AAV vector can be an AAV vector having internal terminal repeats on the 5′ end and the 3′ end of the linear vector with the nucleotide coding for the gene of interest or polypeptide of interest in the form of single stranded or double stranded DNA. In some aspects, such AAV vectors can be used as payloads in combination with mRNA packaged within ionizable/cationic lipids within an LNP wherein the mRNA transiently expresses Rep protein.

I.B.viii Encapsulation Efficacy and Payload Ratios

In some aspects, the encapsulation efficiency of a payload (e.g., an mRNA) in a LNP of the present disclosure is about 95±5%. In some aspects, the encapsulation efficiency of a payload (e.g., an mRNA) in a LNP of the present disclosure is about 100%. In some aspects, the encapsulation efficiency of a payload (e.g., an mRNA) in a LNP of the present disclosure is at least about 70%, at least about 75%, at least about 80%, at least about 85%, at least about 90%, at least about 95%, at least about 96%, at least about 97%, at least about 98%, at least about 99%. In some aspects, the encapsulation efficiency of a payload (e.g., an mRNA) in a LNP of the present disclosure is about 70%, about 75%, about 80%, about 85%, about 90%, about 95%, about 96%, about 97%, about 98%, or about 99%. In some aspects, the encapsulation efficiency of a payload (e.g., an mRNA) in a LNP of the present disclosure is between about 70% and about 75%, between at least about 75% and about 80%, between about 80% and about 85%, between about 85% and about 90%, between about 90% and about 95%, or between about 95% and about 100%.

In some aspects, the ratio of T cell targeting molecule to payload (w/w) in a T cell targeted delivery system of the present disclosure is between about 0.5 and about 4. Thus, in some aspects, the w/w ratio between the T cell targeting molecules (e.g., antibodies such as bispecific antibodies disclosed herein conjugated to the external surface of the LNP) and a mRNA payload can be between about 0.5 and about 4, e.g., about 1.5. In some aspects, such w/w ratio between, e.g., anti CD3/anti CD8 bispecific antibody and mRNA payload, is about 0.5, about 1, about 1.5, about 2, about 2.5, about 3, about 3.5, or about 4. In some aspects, such w/w ratio between, e.g., anti CD3/anti CD8 bispecific antibody and mRNA payload, is between about 0.5 and about 1.5, between about 1 and about 1.5, between about 1.5 and about 2, between about 2 and about 2.5, between about 2.5 and about 3, between about 3 and about 3.5, between about 3.5 and about 4, between about 1.5 and about 3.5, or between about 2 and about 3.

In some aspects, the average number of T cell targeting molecules (e.g., antibodies such as bispecific antibodies disclosed herein conjugated to the external surface of the LNP) per LNP is between about 1 and about 6. In some aspects, the average number of T cell targeting molecules (e.g., antibodies such as bispecific antibodies disclosed herein conjugated to the external surface of the LNP) per LNP is between about 1 and about 3, between about 1 and about 4, between about 1 and about 5, between about 1 and about 6, between about 2 and about 4, between about 2 and about 5, between about 2 and about 6, between about 3 and about 5, between about 3 and about 6, between about 4 and about 6, between about 1 and about 20, between about 5 and about 10, between about 5 and about 20, or between about 10 and about 20.

In some aspects, the average number of T cell targeting molecules per LNP is between about 20 and about 100 T cell targeting molecules per LNP, for example, between about 20 and about 30, between about 30 and about 40, between about 40 and about 50, between about 50 and about 60, between about 60 and about 70, between about 70 and about 80, between about 80 and about 90, between about 90 and about 100, between about 10 and about 30, between about 20 and about 40, between about 30 and about 50, between about 40 and about 60, between about 50 and about 70, between about 60 and about 80, between about 70 and about 90, between about 80 and about 100, between about 10 and about 40, between about 20 and about 50, between about 30 and about 60, between about 40 and about 70, between about 50 and about 80, between about 60 and about 90, between about 70 and about 100, between about 10 and about 50, between about 20 and about 60, between about 30 and about 70, between about 40 and about 80, between about 50 and about 90, between about 60 and about 100, between about 10 and about 60, between about 20 and about 70, between about 30 and about 80, between about 40 and about 90, between about 50 and about 100, between about 10 and about 70, between about 20 and about 80, between about 30 and about 90, between about 40 and about 100, between about 10 and about 80, or between about 20 and about 100.

In some aspects, the average number of T cell targeting molecules (e.g., antibodies such as bispecific antibodies disclosed herein conjugated to the external surface of the LNP) per LNP is 1, 2, 3, 4, 5, 6, 7, 8, 9, 10, 11, 2, 13, 14, about 15, about 20, about 25, about 30, about 35, about 40, about 45, about 50, about 55, about 60, about 65, about 70, about 75, about 80, about 85, about 90, about 95 or about 100. In some aspects, the average number of payload molecules (e.g., mRNA) per LNP is between 1 and 3. In some aspects, the average number of payload molecules (e.g., mRNA) per LNP is 1, 2, 3, 4 or 5. In some aspects, the ratio of targeting molecule (e.g., antibody) to payload (e.g., an mRNA) in μg/μg is about 3, about 4, about 5, about 6, about 7, about 8, about 9, about 10, or about 11. In some aspects, the ratio of targeting molecule (e.g., antibody) to payload (e.g., an mRNA) in μg/μg is between about 3 and about 4, between about 4 and about 5, between about 5 and about 6, between about 6 and about 7, between about 7 and about 8, between about 8 and about 9, between about 9 and about 10, or between about 10 and about 11. In some aspects, the percentage of encapsulated payload (e.g., mRNA) after targeting molecule (e.g., antibody) conjugation is about 80%, about 85%, about 90%, or about 95%. In some aspects, the percentage of encapsulated payload (e.g., mRNA) after targeting molecule (e.g., antibody) conjugation is between about 75% and about 80%, between about 80% and about 85%, between about 85% and about 90%, between about 90% and about 95%, or between about 95% and about 100%.

### I.B.ix Targeting Molecule Conjugation: Maleimide Conjugation

Targeting molecules (e.g., monospecific or bispecific anti-CD3 or anti-CD28 antibodies) can be conjugated to the surface of a LNP of the present disclosure using any of a number of methods known in the art. Thus, the incorporation of a targeting molecule, e.g., a molecule that specifically targets a LNP disclosed herein to a specific cell type or tissue such as the T cell targeting molecules disclosed herein to the surface of a LNP can take place using a conjugation approach know in the art, e.g., those disclosed in Adhikari et al. (2019) Meth. Mol. Biol. 2078:51-69; or McPherson & Hobson (2019) Meth. Mol. Biol. 2078:23-36, which are incorporated herein by reference in their entireties. In some aspects of the present disclosure, the targeting molecules are conjugated to the surface of the LNP using maleimide chemistry.

The present disclosure provides LNPs that have been engineered by reacting a first molecular entity comprising a maleimide group with a second molecular entity comprising a free thiol group, wherein the maleimide moiety covalently links the first molecular entity (e.g., a chemically modified lipid such as DMG-PEG2000-maleimide or DSPE-PEG2000-maleimide) with the second molecular entity (e.g., an antibody) via a maleimide moiety. Thus, in some aspects, the T cell targeted delivery systems of the present disclosure comprise a T cell targeting molecule (e.g., an antibody that specifically binds to a T cell specific surface protein such as CD3 and/or CD28) covalently attached to the LNP via a maleimide moiety.

In some aspects, the maleimide moiety if formed by reaction of maleimide group present on to the surface of the LNP and a thiol group present on the T cell targeting molecule, e.g., a bispecific antibody disclosed herein. In some aspects, the maleimide moiety if formed by reaction of a maleimide group present on the T cell targeting molecule e.g., a bispecific antibody disclosed herein, and a thiol group present on the surface of the LNP, e.g., as part of a modified lipid such as DMG-PEG2000-SH or DSPE-PEG2000-SH.

In some aspects, the maleimide group present on to the surface of the LNP is a reactive group covalently linked to a lipid. In some aspects, the lipid is a phospholipid, e.g., DSPE. In some aspects, the lipid is a phosphoglycerol, e.g., DME. In some aspects, the maleimide group is covalently linked to the lipid via a spacer interposed between the maleimide group and the lipid. In some aspects, the spacer is a water-soluble biopolymer such as polyethylene glycol (PEG), polyglycerol (PG), or poly(propylene glycol) (PPG). In some aspects, the lipid is a PEG-lipid, and the modified lipid is therefore a modified PEG-lipid such as DSPE-PEG2000-maleimide or DMG-PEG2000-maleimide.

As used herein the term “maleimide moiety” refers to a chemical moiety linking an LNP to a linker or a biologically active molecule, e.g., a T cell targeting molecule (e.g., an antibody that specifically binds to a T cell specific surface protein such as CD3 and/or CD28) and comprises the maleimide group:

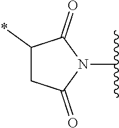 [SMILES: *C1CC(=O)N(*)C1=O]

wherein * indicates the attachment point to any thiol group (e.g., a free SH in an antibody that specifically binds to a T cell specific surface protein such as CD3 and/or CD28), and the wavy line indicates the attachment site to the rest of the molecule comprising maleimide moiety, e.g., the PEG2000 in DSPE-PEG2000-maleimide or DMG-PEG2000-maleimide. In some aspects, * indicates at attachment point to any thiol group on a component of the LNP (e.g., the SH in PEG2000 in DSPE-PEG2000-SH or DMG-PEG2000-SH, or SH in cholesterol-SH) and the wavy line indicates the attachment site to the rest of the maleimide moiety to a T cell targeting molecule (e.g., an antibody that specifically binds to a T cell specific surface protein such as CD3 and/or CD28), for example, via a linker or spacer.

In some aspects, the maleimide moiety attaches to a sulfur atom attached to a T cell targeting molecule (e.g., an antibody that specifically binds to a T cell specific surface protein such as CD3 and/or CD28), e.g., a naturally occurring sulfur atom in a thiol group or a sulfur atom introduced via chemical modification or via mutation.

### In Some Aspects, the Maleimide Moiety has the Formula (I):

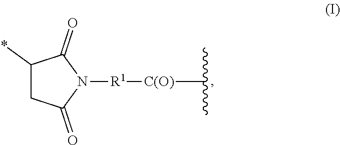

wherein (i) R¹ is selected from the group consisting of —C₁₋₁₀ alkylene-, —C₃₋₈ carbocyclo-, —O—(C₁₋₈ alkylene)-, -arylene-, —C₁₋₁₀ alkylene-arylene-, -arylene-C₁₋₁₀ alkylene-, —C₁₋₁₀ alkylene-(C₃₋₈ carbocyclo)-, —(C₃₋₈ carbocyclo)-C₁₋₁₀ alkylene-, —C₃₋₈ heterocyclo-, —C₁₋₁₀ alkylene-(C₃₋₈ heterocyclo)-, —(C₃₋₈ heterocyclo)-C₁₋₁₀ alkylene-, —(CH₂CH₂O)ᵣ—, and —(CH₂CH₂O)ᵣ—CH₂—; (ii) r is an integer, e.g., from 1 to 10; (iii) * indicates the attachment point to any available reactive sulfur atom, e.g., a sulfur in a thiol group, present on a T cell targeting molecule (e.g., an antibody that specifically binds to a T cell specific surface protein such as CD3 and/or CD28); and, (iv) the wavy line indicates the attachment site of the maleimide moiety to the PEG2000 spacer in DSPE-PEG2000-maleimide or DMG-PEG2000-maleimide.

In some aspects, R¹ is —C₁₋₈ alkylene-, —C₃₋₆ carbocyclo-, —O—(C₁₋₆ alkylene)-, -arylene-, —C₁₋₈ alkylene-arylene-, -arylene-C₁₋₈ alkylene-, —C₁₋₈ alkylene-(C₃₋₆ carbocyclo)-, —(C₃₋₆ carbocyclo)-C₁₋₈ alkylene-, —C₃₋₆ heterocyclo-, —C₁₋₈ alkylene-(C₃₋₆ heterocyclo)-, —(C₃₋₆ heterocyclo)-C₁₋₈ alkylene-, —(CH₂CH₂O)ᵣ—, and —(CH₂CH₂O)ᵣ—CH₂—; where r is an integer, e.g., from 1 to 10. In some aspects, R¹ is —(CH₂)ₛ—, cyclopentyl, cyclohexyl, —O—(CH₂)ₛ—, -phenyl-, —CH₂-phenyl-, -phenyl-CH₂—, —CH₂-cyclopentyl-, -cyclopentyl-CH₂—, —CH₂-cyclohexyl-, -cyclohexyl-CH₂—, —(CH₂CH₂O)ᵣ—, and —(CH₂CH₂O)ᵣ—CH₂—; where r is an integer, e.g., from 1 to 6. In some aspects, R¹ is —(CH₂)ₛ—, wherein s is, e.g., 4, 5, or 6.

In some aspects, the maleimide moiety has the formula (II), wherein R¹ is —(CH₂)₅—:

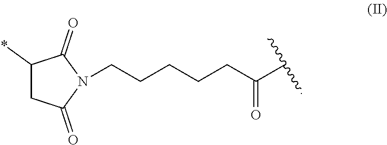

In some aspects, the maleimide moiety has the formula (III), wherein R¹ is —(CH₂CH₂O)ᵣ—CH₂—, and wherein r is 2:

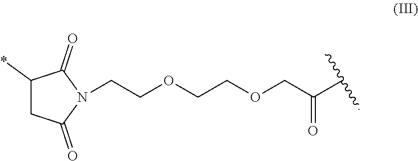

In some aspects, the maleimide moiety is covalently linked to a functional group present on a T cell targeting molecule (e.g., an antibody that specifically binds to a T cell specific surface protein such as CD3 and/or CD28), wherein the functional group is a sulfhydryl (thiol) group. In some aspects, the sulfhydryl group is on a lipid or other LNP component located on the surface of the LNP. For example, in some aspects, the sulfhydryl group can be present on a thiol lipid, e.g., cholesterol-SH, DSPE-SH, or derivatives thereof, e.g., cholesterol-PEG-SH or DSPE-PEG-SH.

In some aspects, the maleimide moiety is covalently linked to a functional group present on a T cell targeting molecule (e.g., an antibody that specifically binds to a T cell specific surface protein such as CD3 and/or CD28) or LNP component (e.g., a lipid) which has been chemically derivatized to provide a maleimide moiety. For example, in some aspects, an amine functional group present on the LNP can be derivatized with a bifunctional reagent comprising, e.g., a succinimide moiety and a maleimide moiety.

In some aspects, a carboxyl functional group present on a T cell targeting molecule (e.g., an antibody that specifically binds to a T cell specific surface protein such as CD3 and/or CD28) or LNP component (e.g., a lipid) can be derivatized with a bifunctional reagent comprising, e.g., an isocyanate moiety and a maleimide moiety.

In some aspects, a carbonyl (oxidized carbohydrate) present on a T cell targeting molecule (e.g., an antibody that specifically binds to a T cell specific surface protein such as CD3 and/or CD28) or LNP component (e.g., a lipid) can be derivatized with a bifunctional reagent comprising, e.g., a hydrazine moiety and a maleimide moiety.

In general, the methods disclosed herein can be practiced using any reagent, e.g., a bifunctional or multifunctional reagent, that upon reacting with a T cell targeting molecule (e.g., an antibody that specifically binds to a T cell specific surface protein such as CD3 and/or CD28) or LNP component molecule (e.g., a lipid) present on the surface (external surface or luminal surface) of the LNP will covalently or non-covalently modify the molecule to yield a modified molecule comprising at least one maleimide moiety.

As used herein, the term “linking” or “attaching” a biologically active molecule, e.g., a T cell targeting molecule (e.g., an antibody that specifically binds to a T cell specific surface protein such as CD3 and/or CD28) to external surface of a LNP of the present disclosure includes both (i) “chemically linking” or “conjugating” the biologically active molecule, e.g., via a chemical linker such as a maleimide moiety, and (ii) “non-chemically linking,” also referred to as “fusing,” or “fusion” of (e.g., via a peptide bond, an amino acid linker, and/or a scaffold protein) the biologically active molecule to the LNP. As used herein, the terms “fusing,” “fused,” “fusion,” or “non-chemically linking” a biologically active molecule on the luminal or external surface of an LNP of the present disclosure via, e.g., a scaffold protein, refers to linking the biologically active molecule to the portion of the scaffold molecule (e.g., protein) located on the luminal or external surface of the LNP, respectively. In some aspects, the fusion between a biologically active molecule can be done via genetic fusion (i.e., chimeric expression).

As used herein, the terms “chemically linking” and “conjugating” are used interchangeably an each refer to the covalent attachment of two or more moieties, each one comprising, e.g., a LNP, a scaffold moiety, a biologically active moiety, a linker or linkers, a targeting moiety and/or a tropism moiety, or any combination thereof, using a chemical moiety, e.g., a maleimide moiety. As a result, a first moiety would become “chemically linked” to a second moiety, e.g., a biologically active moiety, via a thioether linkage formed by the reaction between the maleimide group present in one moiety and a sulfhydryl group present in the other moiety.

The LNP of the present disclosure can comprise one or more linkers that link (i.e., connect) the maleimide moiety to the biologically active molecule or to the LNP. In some aspects, the maleimide moiety is linked to the biologically active molecule by a linker. The linker can be any chemical moiety capable of, e.g., linking a maleimide moiety, e.g., of formula (II) or (III), to a biologically active molecule. In some aspects, a maleimide moiety can comprise one or more linkers. In some aspects, the linkers disclosed herein or combinations thereof can be used to connect, e.g., a maleimide moiety to a biologically active molecule, a first biologically active moiety to a second biologically active moiety, a LNP to a maleimide moiety, or a LNP to a biologically active moiety.

In some aspects, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present in a maleimide moiety disclosed herein, each of the linkers can be the same or different. Generally, linkers provide flexibility or prevent/ameliorate steric hindrances. Linkers are not typically cleaved; however in some aspects, such cleavage can be desirable. Accordingly, in some aspects a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids. In some aspects, the peptide linker can comprise about two, about three, about four, about five, about 10, about 15, about 20, about 25, about 30, about 35, about 40, about 45, about 50, about 55, about 60, about 65, about 70, about 75, about 80, about 85, about 90, about 95, or about 100 amino acids. In some aspects, the peptide linker can comprise at least about 110, at least about 120, at least about 130, at least about 140, at least about 150, at least about 160, at least about 170, at least about 180, at least about 190, or at least about 200 amino acids. In some aspects, the peptide linker can comprise about 110, about 120, about 130, about 140, about 150, about 160, about 170, about 180, about 190, or about 200 amino acids. In some aspects, the peptide linker can comprise at least about 200, at least about 300, at least about 400, at least about 500, at least about 600, at least about 700, at least about 800, at least about 900, or at least about 1,000 amino acids. In some aspects, the peptide linker can comprise about 200, about 300, about 400, about 500, about 600, about 700, about 800, about 900, or about 1,000 amino acids. The peptide linker can comprise between 1 and about 5 amino acids, between 1 and about 10 amino acids, between 1 and about 20 amino acids, between about 10 and about 50 amino acids, between about 50 and about 100 amino acids, between about 100 and about 200 amino acids, between about 200 and about 300 amino acids, between about 300 and about 400 amino acids, between about 400 and about 500 amino acids, between about 500 and about 600 amino acids, between about 600 and about 700 amino acids, between about 700 and about 800 amino acids, between about 800 and about 900 amino acids, or between about 900 and about 1000 amino acids.

In some aspects, the linker is a glycine/serine linker. In some aspects, the peptide linker is glycine/serine linker according to the formula [(Gly)n-Ser]m (SEQ ID NO: 530) where n is any integer from 1 to 100 and m is any integer from 1 to 100. In some aspects, the glycine/serine linker is according to the formula [(Gly)x-Sery]z (SEQ ID NO: 531) wherein x in an integer from 1 to 4, y is 0 or 1, and z is an integer from 1 to 50. In some aspects, the peptide linker comprises the sequence Gn, where n can be an integer from 1 to 100. In some aspects, the peptide linker can comprise the sequence (GlyAla)n (SEQ ID NO: 532), wherein n is an integer between 1 and 100. In some aspects, the peptide linker can comprise the sequence (GlyGlySer)n (SEQ ID NO: 533), wherein n is an integer between 1 and 100.

### I.C Targeting Molecules

The present disclosure provides cell- or tissue-specific LNP delivery systems in which at least one targeting molecule is conjugated to the surface of the LNP. The targeting molecules can specifically bind to a target present on the surface of specific cell types, e.g., T cells or cancer cells, or tissues, e.g., endothelium, muscle tissue, CNS, or tumors. Proteins that are suitable as targets for directing payloads via the LNP of the present disclosure to specific tissues, cell types, human cancers, etc. are disclosed, e.g., at the Human Protein Atlas, available at proteinatlas.org (Version 23.0, Release date: 2023.06.19, Ensembl version: 109); the National Cancer Institute list of tumors markers in common use available at www.cancer.gov/about-cancer/diagnosis-staging/diagnosis/tumor-markers-list; Ryboshapkina & Hamma (2019) “Tissue-specific genes as an underutilized resource in drug discovery” Sci. Rep. 9:7233; or US20230203199A1 which are herein incorporated by reference in their entireties. In some aspects, a targeting molecule comprises an antibody or antigen-binding portion thereof disclosed in Section I.B.iv (Antibodies), see above.

In some aspects, at least one T cell targeting molecule in a T cell targeted delivery system comprises an antigen-binding molecule, wherein the antigen is specifically binds to a T cell specific surface protein. As described above a T cell targeted delivery system of the present disclosure specifically binds to at least two T cell specific surface proteins. Thus, in some aspects, the T cell targeted delivery system comprises a bispecific or multispecific antigen-binding molecule capable of specifically binding to at least two T cell specific surface proteins (e.g., CD3 and CD28). In some aspects, the T cell targeted delivery system comprises a set of monospecific (monovalent, bivalent, or multivalent) antigen-binding molecules capable of specifically binding to at least two T cell specific surface proteins (e.g., CD3 and CD28). In some aspects, the antigen-binding molecules used in a T cell targeted delivery system of the present disclosure are antibodies, or at least comprise an antibody. In some aspects, the antibody is IgG, IgM, IgE, IgA or IgD. In some aspects, the IgG is IgG1, IgG2, IgG3 or IgG4. In some aspects, at least one T cell targeting molecule in a T cell targeted delivery system of the present disclosure comprises a Fab, scFab, Fab′, F(ab′)₂, Fv, or scFv. In some aspects, the antibody is human or humanized.

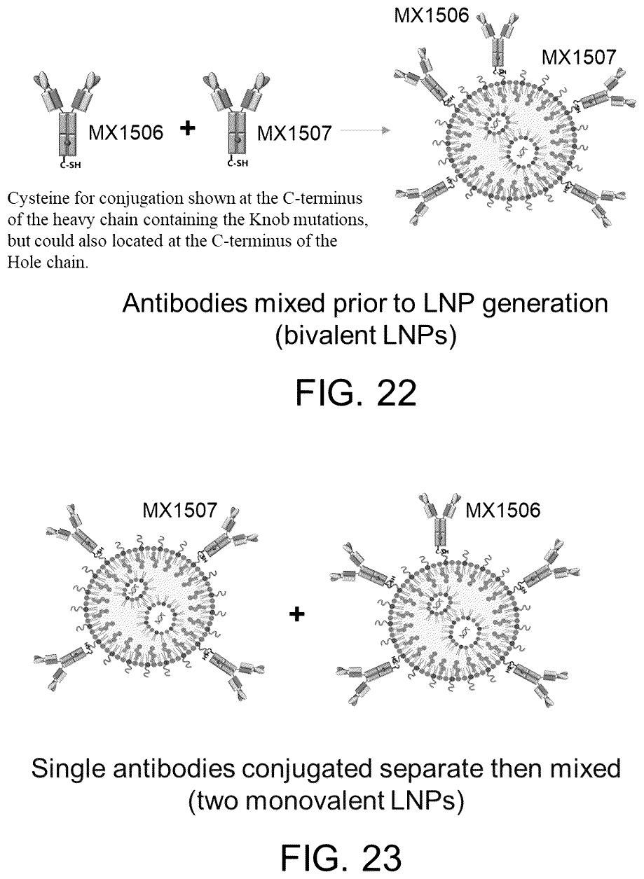

In some aspects, the claim T cell targeting molecule(s) above a T cell targeted delivery system of the present disclosure comprise(s) (i) a monospecific monovalent antibody; (ii) a monospecific bivalent or multivalent antibody; (iii) a bispecific antibody; or, (iv) a combination thereof. In some aspects, the T cell targeting molecule(s) comprise(s) an antibody or combination thereof selected from the formats presented in FIG. 1A, FIG. 1B, and FIG. 22.

In some aspects, the T cell targeting molecule comprises a bispecific antibody targeting two T cell specific surface proteins (e.g., CD3 and CD28). Thus, in some aspects, the T cell targeting molecule is an anti-CD3/anti-CD28 antibody. In some aspects, the T cell targeting molecule(s) comprise(s) (i) a bispecific anti-CD3/anti-CD28 antibody; (ii) a monospecific-monovalent anti-CD3 antibody and a monospecific-monovalent anti-CD28 antibody; (iii) a monospecific-bivalent anti-CD3 antibody and a monospecific-monovalent anti-CD28 antibody; (iv) a monospecific-monovalent anti-CD3 antibody and a monospecific-bivalent anti-CD28 antibody; or, (v) a monospecific-bivalent anti-CD3 antibody and a monospecific-bivalent anti-CD28 antibody. In some aspects, the T cell targeting molecule comprises a bispecific anti-CD3/anti-CD28 antibody having a structure selected from Format C, Format D, Format E and Format F of FIG. 1B, or the formats presented in FIG. 22.

### I.C.i Bispecific Antibody MSTAR Architectures

In some aspects of the T cell targeted delivery system of the present disclosure, the T cell targeting molecule comprises, consists, or consists essentially of an antigen binding polypeptide that specifically binds to a first T cell specific surface protein and to a second T cell specific surface protein, wherein the antigen binding polypeptide has a structure represented by: VL1-VL2-VH2-VH1; VH1-VH2-VL2-VL1; VL1-L1-VL2-L2-VH2-L3-VH1; or VH1-L1-VH2-L2-VL2-L3-VL1; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; and L1, L2 and L3 are amino acid linkers.

In some aspects of the T cell targeted delivery system of the present disclosure, the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by: VL1-VL2-VH2-VH1; VH1-VH2-VL2-VL1; VL1-L1-VL2-L2-VH2-L3-VH1; or VH1-L1-VH2-L2-VL2-L3-VL1; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; and L1, L2 and L3 are amino acid linkers.

In some aspects of the T cell targeted delivery system of the present disclosure, the T cell targeting molecule comprises, consists, or consists essentially of an antigen binding polypeptide that specifically binds to a first T cell specific surface protein and to a second T cell specific surface protein having a structure represented by: VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH₂), an immunoglobulin heavy chain constant region 3 (CH₃), and optionally, an immunoglobulin hinge; and L1, L2, L3 and L4 are amino acid linkers.

In some aspects of the T cell targeted delivery system of the present disclosure, the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by: Fc; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or, VH1-L1-VH2-L2-VL2-L3-VL1-L4-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH₂), an immunoglobulin heavy chain constant region 3 (CH₃), and optionally, an immunoglobulin hinge; and L1, L2, L3 and L4 are amino acid linkers.

In some aspects of the T cell targeted delivery system of the present disclosure, the T cell targeting molecule comprises, consists, or consists essentially of an antigen binding polypeptide that specifically binds to a first T cell specific surface protein and to a second T cell specific surface protein having a structure represented by: VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; and L1, L2, L3, L4 and L5 are amino acid linkers.

In some aspects of the T cell targeted delivery system of the present disclosure, the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by: VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; and L1, L2, L3, L4 and L5 are amino acid linkers.

The T cell targeted delivery system of any one of claims 1 to 53, wherein the T cell targeting molecule comprises, consists, or consists essentially of an antigen binding polypeptide that specifically binds to a first T cell specific surface protein and to a second T cell specific surface protein having a structure represented by: VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In some aspects of the T cell targeted delivery system of the present disclosure, the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by: Fc; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In some aspects of the T cell targeted delivery system of the present disclosure, the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by: Fc; VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fe; VH1-L1-VH2-L2-VL2-L3-VL1-L4-CL-L5-CH1-Fe; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In some aspects of the T cell targeted delivery system of the present disclosure, the T cell targeting molecule comprises, consists, or consists essentially of an antigen binding polypeptide or antigen binding polypeptide complex that specifically binds to a first T cell specific surface protein and to a second T cell specific surface protein comprising a polypeptide having a structure represented by: VL1-VL2-VH2-VH1-Fc-Fc; VH1-VH2-VL2-VL1-Fc-Fc; VL1-L1-VL2-L2-VH2-L3-VH1-Fc-Fc; VH1-L1-VH2-L2-VL2-L3-VL1-Fc-Fc; VL1-L1-VL2-L2-VH2-L3-VH1-L4-Fc-Fc; VH1-L1-VH2-L2-VL2-L3-VL1-L4-Fc-Fc; VL1-L1-VL2-L2-VH2-L3-VH1-L4-Fc-L5-Fc; or VH1-L1-VH2-L2-VL2-L3-VL1-L4-Fc-L5-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH2), an immunoglobulin heavy chain constant region 3 (CH3), and optionally, an immunoglobulin hinge; and L1, L2, L3, L4 and L5 are amino acid linkers.

In some aspects of the T cell targeted delivery system of the present disclosure, the T cell targeting molecule comprises, consists, or consists essentially of an antigen binding polypeptide or antigen binding polypeptide complex that specifically binds to a first T cell specific surface protein and to a second T cell specific surface protein comprising a polypeptide having a structure represented by: VL1-VL2-VH2-VH1-CH3; VH1-VH2-VL2-VL1-CH3; VL1-L1-VL2-L2-VH2-L3-VH1-CH3; VH1-L1-VH2-L2-VL2-L3-VL1-CH3; VL1-L1-VL2-L2-VH2-L3-VH1-L4-CH3; VH1-L1-VH2-L2-VL2-L3-VL1-L4-CH3; VL1-VL2-VH2-VH1-CH3-CH3; VH1-VH2-VL2-VL1-CH3-CH3; VL1-L1-VL2-L2-VH2-L3-VH1-CH3-CH3; VH1-L1-VH2-L2-VL2-L3-VL1-CH3-CH3; VL1-L1-VL2-L2-VH2-L3-VH1-L4-CH3-CH3; VH1-L1-VH2-L2-VL2-L3-VL1-L4-CH3-CH3; VL1-L1-VL2-L2-VH2-L3-VH1-L4-CH3-L5-CH3; or VH1-L1-VH2-L2-VL2-L3-VL1-L4-CH3-L5-CH3; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH3 is an immunoglobulin heavy chain constant region 3; and L1, L2, L3, L4 and L5 are amino acid linkers.

TABLE 7
Sequences of possible VH and VL components of the bispecific
structures disclosed above. Each bispecific structure comprises
at least a VH1, a VH2, a VL1, and a VL2, wherein at least
one VH/VL set is specific for CD3 and at least VH/VL set
is specific for CD28. CDRs are underlined.

| | | | | CDR1, CDR2, |
|---|---|---|---|---|
| | | | VH/VL | CDR3 |
| | Antibody S | | SEQ | SEQ |
| Component | pecificity | Description | ID NO | ID NOS |
| VH1/VH2 | Human CD3 | aCD3_vH | 40 | 46, 47, 48 |
| VL1/VL2 | Human CD3 | aCD3_vL | 41 | 49, 50, 51 |
| VH1/VH2 | Human CD28 | aCD28_vH | 42 | 52, 53, 54 |
| VL1/VL2 | Human CD28 | aCD28_vL | 43 | 55, 56, 57 |
| VH1/VH2 | CD3 | SARCD3_vH | 44 | 58, 59, 60 |
| VL1/VL2 | CD3 | SARCD3_vL | 45 | 61, 62, 63 |

TABLE 8
Linkers. Sequences of possible linkers for the bispecific structures
disclosed above. Each one of the L1-L6 linkers in the bispecific
structures presented above can be independently selected from the
linkers in the table and other linker disclosed in the specification.

| | Sequence or |
|---|---|
| Component | SEQ ID NO |
| L1-L6 | G |
| L1-L6 | A |
| L1-L6 | GSS |
| L1-L6 | ASG |
| L1-L6 | 109 |
| L1-L6 | 110 |
| L1-L6 | 111 |
| L1-L6 | 112 |
| L1-L6 | 113 |
| L1-L6 | 114 |
| L1-L6 | 115 |
| L1-L6 | 116 |
| L1-L6 | 117 |
| L1-L6 | 118 |
| L1-L6 | 119 |
| L1-L6 | 120 |
| L1-L6 | 121 |
| L1-L6 | 122 |
| L1-L6 | 123 |
| L1-L6 | 124 |
| L1-L6 | 125 |
| L1-L6 | 126 |
| L1-L6 | 127 |
| L1-L6 | 128 |
| L1-L6 | 129 |
| L1-L6 | 130 |
| L1-L6 | 131 |

TABLE 9
Other components in the bispecific constructs disclosed above.

| Component | SEQ ID NO |
|---|---|
| Fc (LALAPAhole Fc) | 134 |
| Fc (LALAPAhole_C) Fc | 135 |
| Fc (LALAPAknob Fc) | 136 |
| Fc (LALAPAknob_C) | 137 |
| CH1 | 133 |
| CH2 | 139 |
| CH3 | 138 |
| CL | 132 |

In some aspects, an architecture disclosed above comprises two Fc domains comprising mutations that promote heterodimerization, e.g., “knob” or “hole” mutations (disclosed in more detail below). In some aspects, the Fc forming a knob-hole pair have the sequences set forth in SEQ ID NO: 134 (hole) and SEQ ID NO: 136 (knob). In some aspects, the Fc forming a knob-hole pair have the sequences set forth in SEQ ID NO: 135 (hole) and SEQ ID NO: 137 (knob). In some aspects, the Fc component of an architecture disclosed above comprises the “LALAPA mutations.” These mutations (L23A, L235A, P239A) reduce binding to the IgG Fc receptors FcγRI, FcγRII and FcγRIII as well as to complement component C1q. In some aspects, the Fc component comprises other sets of mutations the diminish or abolish effector functions such as L234F/L235E/P331S (FES), L234F/L235Q/K322Q (FQQ), L234A/L235A/P329G (LALAPG). Other sets of mutations known to diminish effector function include, for example, L234A/G237A, L234A/L235A/G237A, L234A/L235A/G237A/P238S/H268A/A330S/P330S, L234A/L235E, G236R/L328R, and L234A/L235A/K322A.

In some aspects, VH1, VH2, VL1 and VL2 bind to the same type of T cell specific surface protein, e.g., either CD3 or CD28. In other words, in some aspects, the first T cell specific surface protein and the second T cell specific surface protein are the same protein. Accordingly, in some aspects, the architectures presented above can represent monospecific antibodies.

In some aspects, VH1/VL1 and VH2/VL2 bind to the same epitope on the same protein, e.g., the same epitope on CD3 or CD28. In some aspects, VH1/VL1 and VH2/VL2 bind to the different epitopes on the same protein, e.g., the same epitope on CD3 or CD28.

In some aspects, one or more of the VH1 comprises an amino acid sequence having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to a VH of SEQ ID NO: 40 or 44 and/or one or more of the VH2 comprises an amino acid sequence having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to a VH of SEQ ID NO: 42.

In some aspects, one or more of the VH1 comprises an amino acid sequence having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to a VH of SEQ ID NO: 42 and/or one or more of the VH2 comprises an amino acid sequence having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to a VH of SEQ ID NO: 40 or 44.

In some aspects, one or more of the VL1 comprises an amino acid sequence having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to a VL of SEQ ID NO: 41 or 45 and/or one or more of the VL2 comprises an amino acid sequence having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to a VL of SEQ ID NO: 43.

In some aspects, one or more of the VL1 comprises an amino acid sequence having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% identity to a VL of SEQ ID NO: 43 and/or one or more of the VL2 comprises an amino acid sequence having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to a VL of SEQ ID NO: 41 or 45.

In some aspects, the antigen binding polypeptide or antigen binding polypeptide complex comprises an immunoglobulin hinge comprising or consisting of an upper hinge region, a middle hinge region, a lower hinge region, or a combination thereof.

In some aspects, the antigen binding polypeptide or antigen binding polypeptide complex comprises one or more amino acid linkers between two or more regions of the antigen binding polypeptide or polypeptide complex.

As used herein, an “amino acid linker” can be a single amino acid or short amino acid sequence that is capable of joining two regions of an antigen binding polypeptide or polypeptide complex provided herein in a stable manner that maintains or promotes a function associated with the regions. However, as used herein, an “amino acid linker” can also be included where the amino acid linker is not present between two regions, i.e., in some aspects, an amino acid linker L1 to L6 can have 0 amino acids.

In some aspects, an amino acid linker is represented herein in a structure of an antigen binding polypeptide or polypeptide complex by the abbreviation “1” or “L” and a number (e.g., L1 to denote a first linker, L2 to denote a second linker, L3 to denote a third linker, L4 to denote a fourth linker, L5 to denote a fifth linker, and L6 to denote a sixth linker). In some aspects, such enumerated amino acid linkers (e.g., L1) can have the same or different sequence as any other enumerated amino acid linker (e.g., L2, etc.).

In some aspects, an amino acid linker has a length of from 0 amino acids (i.e., an amino acid linker is not present) to about 50 amino acids (e.g., one or more of L1, L2, L3, L4, L5, and L6 or more of a first and/or second polypeptide of an antigen binding polypeptide or polypeptide complex structure provided herein). In some aspects, the amino acid linker has a length of from 0 amino acids to about 45 amino acids, 0 amino acids to about 40 amino acids, 0 amino acids to about 35 amino acids, 0 amino acids to about 30 amino acids, 0 amino acids to about 25 amino acids, 0 amino acids to about 20 amino acids, 0 amino acids to about 15 amino acids, 0 amino acids to about 10 amino acids, 0 amino acids to about 5 amino acids, about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, 1 amino acid to about 15 amino acids, about 1 amino acid to about 10 amino acids, about 1 amino acid to about 5 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 40 amino acids to about 50 amino acids, about 40 amino acids to about 45 amino acids, or about 45 amino acids to about 50 amino acids

In some aspects, the amino acid linker has 0 amino acids (i.e., an amino acid linker is not present) or about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50 amino acids (e.g., one or more of L1, L2, L3, L4, L5, or L6 of a first and/or second polypeptide of an antigen binding polypeptide or polypeptide complex structure provided herein).

In some aspects, the amino acid linker consists of one or more amino acid residues (e.g., one or more of L1, L2, L3, L4, L5, or L6 of a first and/or second polypeptide of an antigen binding polypeptide or polypeptide complex structure provided herein). In some aspects, the amino acid residues are selected from the group consisting of glycine, alanine, serine, threonine, cysteine, asparagine, glutamine, leucine, isoleucine, valine, proline, histidine, aspartic acid, glutamic acid, lysine, arginine, methionine, phenylalanine, tryptophan, and tyrosine.

In some aspects, an amino acid linker is non-immunogenic. In some aspects, a non-immunogenic linker consists of serine, glycine and/or alanine residues, or consists of serine and/or glycine residues. In some aspects, an amino acid linker does not contain a T cell epitope or consensus T cell epitope. In some aspects, an amino acid linker consists of one or more residues of alanine, cysteine, glycine, isoleucine, leucine, methionine, phenylalanine, proline, tryptophan, tyrosine, or valine (e.g., one or more of L1, L2, L3, L4, L5, or L6 of a first and/or second polypeptide of an antigen binding polypeptide or polypeptide complex structure provided herein).

Amino acid linker sequences that can be used with the antigen binding polypeptides or polypeptide complexes (e.g., an antibody or antigen binding fragment thereof) provided herein are well known and can be incorporated into the antigen binding polypeptides and polypeptide complexes provided herein using routine molecular biology and recombinant DNA techniques. See, e.g., Chen et al., Adv Drug Deliv Rev., 65(10):1357-1369, 2013; and Chichili et al., Protein Sci., 22(2):153-167, 2013. In some aspects, the linkers (e.g., L1, L2, L3, L4, L5, and/or L6) have independently a length of from about 1 amino acid to about 50 amino acids. In some aspects, each linker L1, L2, L3, L4, L5, and/or L6 independently comprises or consists of the amino acid sequence of G, A, GSS, ASG, SEQ ID NOS: 109-131 or a sequence having at least about 50% sequence identity, at least about 60% sequence identity, at least about 70% sequence identity, at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or 100% identity to any one of SEQ ID NOS: 109-131, or a subsequence thereof.

In some aspects, the amino acid linker between VL1 and VL2 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 110. In some aspects, the amino acid linker between VL2 and VH2 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 131.

In some aspects, the amino acid linker between VH2 and VH1 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 110. In some aspects, the amino acid linker between VH1 and Fc comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 116.

In some aspects, the amino acid linker between VL1 and VL2 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 110; the amino acid linker between VL2 and VH2 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 131; the amino acid linker between VH2 and VH1 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 110; and the amino acid linker between VH1 and Fc comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 116.

In some aspects, the amino acid linker between CL and CH1 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 131.

In some aspects, the amino acid linker between VL1 and VL2 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 110; the amino acid linker between VL2 and VH2 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 131; the amino acid linker between VH2 and VH1 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 110; and the amino acid linker between CL and CH1 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 131.

In some aspects, the amino acid linker between CH1 and Fc comprises 0 amino acids. In some aspects, the amino acid linker between VL1 and VL2 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 110; the amino acid linker between VL2 and VH2 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 131; the amino acid linker between VH2 and VH1 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 110; and the amino acid linker between CL and CH1 comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99% or about 100% sequence identity to the sequence of SEQ ID NO: 131; and the amino acid linker between CH1 and Fc comprises 0 amino acids.

### I.C.ii Surface Anchored Targeting Molecule Modifications

In some aspects, the surface anchored targeting molecules, e.g., antibodies, contain modifications to improve their function.

### I.C.ii.a Effector and Half-Life Modifications

In some aspects, an antigen binding polypeptide or polypeptide complex (e.g., an antibody or antigen binding fragment thereof) provided herein comprises an effector function mutation or half-life extension mutation.

Effector functions are an important part of the humoral immune response and form a link between innate and adaptive immunity. Most effector functions are induced via the Fc region of an antibody, which can interact with complement proteins and specialized Fc receptors. As used herein, an “effector function mutation” or “Fc effector function mutation” refer to a change in the amino acid sequence, typically in the Fc region, which can increase or decrease effector function, for example, increase binding affinity of Fc for specific Fc receptors, or increase antibody-dependent cellular cytotoxicity (ADCC) activity.

“Half-life” of a pharmaceutically active substance is the time it takes for the amount of the substance, once administered to the body, to reduce by half. A “half-life extension mutation” of an antigen binding polypeptide or polypeptide complex provided herein refers to a change in the amino acid sequence, typically in the Fc region, which increases the half-life of the antigen binding polypeptide or polypeptide complex (e.g., by increasing Fc receptor binding affinity, slowing off-rate for Fc and Fc receptors, and/or increased sialylation). Examples of Fc effector function mutations that decrease or knockout function include, but are not limited to, the following substitutions in the Fc region, based on the EU numbering scheme: L234A, L235A, P239A, N297A, or a combination thereof. Examples of Fc effector function mutations that increase function include, but are not limited to, the following substitutions in the Fc region, based on the EU numbering scheme: S298A/E333A/K334A, S239D/I332E, S239D/A330L/I332E, G236A/S239D/I332E, or a combination thereof. Additional examples of effector function mutations, half-life extension mutations and methods for incorporating the same into an amino acid sequence are known and described, for example, in Saunders, “Conceptual Approaches to Modulating Antibody Effector Functions and Circulation Half-Life,” Front. Immunol. Jun. 7, 2019; U.S. Pat. No. 8,546,543, and U.S. Pub. No. 2020/0054765.

### I.C.ii.b Heterodimer-Forming Modifications

In some aspects, an antigen binding polypeptide or polypeptide complex (e.g., an antibody or antigen binding fragment thereof) provided herein comprises at least one heterodimer-forming modification. In some aspects, the interaction between the polypeptides of an antigen binding polypeptide complex comprises at the interface of the heterodimerization domains a protuberance-into-cavity interaction, a hydrophobic interaction and/or an electrostatic interaction.

In some aspects, the heteromultimerization domain comprises a knob (e.g., protuberance), a hole (e.g., cavity), a leucine zipper, a coiled coil, or a polar amino acid residue capable of forming an electrostatic interaction, or combinations thereof.

In some aspects, the first polypeptide comprises a knob, and the second polypeptide comprises a hole. In some aspects, the interaction involves both a hydrophobic interaction and an electrostatic interaction. In some aspects, the heteromultimerization domain of each of the first and second polypeptides comprises either a knob or a hole and an amino acid residue capable of forming an electrostatic interaction.

It is understood by one skilled in the art that the heterodimerization domain can comprise more than one way of interaction selected, for example, from the group consisting of knob-into-hole modification (KIH), electrostatic steering modification (ESS), protein A affinity-altering modification, hydrophobic interaction, leucine zipper, coiled coil, or a combination thereof.

The term “knob-into-hole modification,” as used herein, refers to a genetic modification that directs the pairing of two polypeptides to promote heterodimerization. In some aspects, the modification introduces a protuberance (knob) into one polypeptide and a cavity (hole) into the other polypeptide at an interface in which the two polypeptides interact. In some aspects, a knob-into-hole modification can be created by introducing only a hole modification, for example, by replacing an amino acid residue with a smaller side chain than the original amino acid residue (e.g., a substitution of one or more serine, threonine, valine or alanine residues, or a combination thereof). In some aspects, a knob-into-hole modification can be created by introducing only a knob modification, for example, by replacing an amino acid residue with a larger side chain than the original amino acid residue (e.g., a substitution of one or more tryptophan or tyrosine residues, or a combination thereof).

In some aspects, the knob-into-hole modification is in the binding interface of two Fc regions, the binding interface of two CH2 regions, the binding interface of two CH3 regions, the binding interface of a CL region and a CH1 region, or the binding interface of a VH region and a VL region. See, e.g., U.S. Pub. No. 2007/0178552, Int'l Pub. No. WO 96/027011, Int'l Pub. No. WO 98/050431 and Zhu et al., Prot. Sci. 6:781-788, 1987, which are herein incorporated by reference.

In some aspects, the antigen binding polypeptide or polypeptide complex comprises one, two, three, four, five, six, seven, eight, nine, ten, or more knob-into-hole modifications.

Knob-into-hole modifications are well known and can be incorporated into antigen binding polypeptides and polypeptide complexes provided herein using routine molecular biology and recombinant DNA techniques. See, e.g., U.S. Pub. No. 2003/0078385; Int'l Pub. No. WO 96/027011; Ridgway et al., Protein Eng., 9:617-621, 1996; and Merchant et al., Nat. Biotechnol., 16:677-681, 1998, which are herein incorporated by reference. In some aspects, the knob-into-hole modification is an amino acid substitution. Such a substitution is described based on the EU numbering scheme of Kabat, which corresponds to the numbering in the Protein Data Bank (PDB).

In some aspects, the knob-into-hole modifications are M428L and N434S or N434A, based on the EU numbering scheme. In some aspects, the knob-into-hole modifications are M428L and N424S, based on the EU numbering scheme. In some aspects, the knob-into-hole modification is N434A, based on the EU numbering scheme. In some aspects, the knob-into-hole modification is a knob substitution of S354C and/or T366W, based on the EU numbering scheme. In some aspects, the knob-into-hole modification is a knob substitution of S354C, T366W, M428L and N434S, or N434A based on the EU numbering scheme. In some aspects, the knob-into-hole modification is a hole substitution of Y349C, T366S, L368A, Y407V, M428L, N434S, N434A, M252Y, S254T, T256E, or a combination thereof, based on the EU numbering scheme. In some aspects, the knob-into-hole modifications are hole substitutions of Y349C, T366S, L368A and Y407V, based on the EU numbering scheme. In some aspects, the knob-into-hole modifications are hole substitutions of M428L and N434S. In some aspects, the knob-into-hole modification is a hole substitution of N434A. In some aspects, the knob-into-hole modifications are hole substitutions of M428L and N434S or N434A, based on the EU numbering scheme. In some aspects, the knob-into-hole modifications are hole substitutions of M252Y, S254T and T256E, based on the EU numbering scheme. In some aspects, the knob-into-hole modifications are hole substitutions of Y349C, T366S, L368A, Y407V, M428L, and N434S.

In some aspects, an antigen binding polypeptide complex is an IgG1 or IgG4 antibody and the knob-into-hole modifications are knob substitutions of S354C and T366W and hole substitutions of Y349C, T366S, L368A and Y407V. In some aspects, the antigen binding polypeptide complex is an IgG1 or IgG4 antibody and the knob-into-hole modifications are hole substitutions of M428L and N434S or N434A. In some aspects, the knob-into-hole modifications are hole substitutions of M428L and N434S. In some aspects, the knob-into-hole modification is a hole substitution of N434A. In some aspects, the antigen binding polypeptide complex is an IgG1 or IgG4 antibody and the knob-into-hole modifications are hole substitutions of M252Y, S254T and T256E.

In some aspects, the modifications or mutations can be both in the Fc region and in the antigen binding polypeptide or antigen binding polypeptide complex. In some aspects, heterodimerization is driven in the bispecific antibody formats disclosed above by knob-into-hole modifications. In some aspects, the knob-into-hole modification comprises: (i) knob substitutions of S354C and T366W and hole substitutions of Y349C, T366S, L368A and Y407V; (ii) hole substitutions of L234A, L235A and P239A; (iii) hole substitutions of L234A and L235A; (iv) hole substitutions of M428L and N433S; (v) hole substitutions of M252Y, S254T and T256E; or (vi) a combination thereof, wherein the amino acid numbering is according to the EU numbering scheme.

### I.C.ii.c Labeling/Tagging

In some aspects, the bispecific antibodies disclosed above comprise an antigen binding polypeptide or antigen binding polypeptide complex comprising a detectable label. In some aspects, the detectable label is a radioactive label, chemiluminescent label, fluorescent label, enzyme, or peptide tag, or a combination thereof. In some aspects, the peptide tag is a polyhistidine tag consisting of from about 4 to about 10 histidine residues. In some aspects, the polyhistidine tag consists of about 8 histidine residues. In some aspects, the polyhistidine tag consists of 4 histidine residues. In some aspects, the polyhistidine tag consists of 5 histidine residues. In some aspects, the polyhistidine tag consists of 6 histidine residues. In some aspects, the polyhistidine tag consists of 7 histidine residues. In some aspects, the polyhistidine tag consists of 8 histidine residues. In some aspects, the polyhistidine tag consists of 9 histidine residues. In some aspects, the polyhistidine tag consists of 10 histidine residues.

### I.C.iii Anti-CD3/CD28 Bispecific Antibody Constructs

In some aspects, the T cell targeting molecule(s) disclosed herein comprise(s) an anti-CD3 VH region comprising VH-CDR1, VH-CDR2 and VH-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 46, 47 and 48, respectively.

In some aspects, the T cell targeting molecule(s) comprise(s) an anti-CD3 VL region comprising VL-CDR1, VL-CDR2 and VL-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 49, 50 and 51, respectively.

In some aspects, the claim T cell targeting molecule(s) comprise(s) an anti-CD28 VH region comprising VH-CDR1, VH-CDR2, and VH-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 52, 53, and 54, respectively.

In some aspects, T cell targeting molecule(s) comprise(s) an anti-CD28 VL region comprising VL-CDR1, VL-CDR2, and VL-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 55, 56, and 57, respectively.

In some aspects, the T cell targeting molecule(s) comprise(s) an anti-CD3 VH region comprising VH-CDR1, VH-CDR2 and VH-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 58, 59 and 60, respectively.

In some aspects, the T cell targeting molecule(s) comprise(s) an anti-CD3 VL region comprising VL-CDR1, VL-CDR2 and VL-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 61, 62 and 63, respectively.

In some aspects, the claim T cell targeting molecule(s) comprise(s) (i) an anti-CD3 VH region comprising VH-CDR1, VH-CDR2 and VH-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 46, 47 and 48, respectively; (ii) an anti-CD3 VL region comprising VL-CDR1, VL-CDR2 and VL-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 49, 50 and 51, respectively; (iii) an anti-CD28 VH region comprising VH-CDR1, VH-CDR2, and VH-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 52, 53, and 54, respectively; (iv) an anti-CD28 VL region comprising VL-CDR1, VL-CDR2, and VL-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 55, 56, and 57, respectively; (v) an anti-CD3 VH region comprising VH-CDR1, VH-CDR2 and VH-CDR3 having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 58, 59 and 60, respectively; (vi) an anti-CD3 VL region comprising VL-CDR1, VL-CDR2 and VL-CDR3 at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NOS: 61, 62 and 63, respectively; or (vii) any combination thereof.

In some aspects, the T cell targeting molecule(s) comprise(s) an anti-CD3 VH region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 40. In some aspects, the T cell targeting molecule(s) comprise(s) an anti-CD3 VL region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 41. In some aspects, the claim T cell targeting molecule(s) comprise(s) an anti-CD28 VH region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 42. In some aspects, the claim T cell targeting molecule(s) comprise(s) an anti-CD28 VL region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 43. In some aspects, the T cell targeting molecule(s) comprise(s) an anti-CD3 VH region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 44. In some aspects, the claim T cell targeting molecule(s) comprise(s) an anti-CD3 VL region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 45.

In some aspects, the claim T cell targeting molecule(s) comprise(s) (i) an anti-CD3 VH region having at least about 80% identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 40; (ii) an anti-CD3 VL region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 41; (iii) an anti-CD28 VH region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 42; (iv) an anti-CD28 VL region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 43; (v) an anti-CD3 VH region having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 44; (vi) an anti-CD3 VL region at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 45; or, (vii) any combination thereof.

In some aspects, the T cell targeted delivery system of the present disclosure comprises a T cell targeting molecule designated MX1501 which comprises a first polypeptide of SEQ ID NO:1, and a second polypeptide of SEQ ID NO: 19. In some aspects, the T cell targeted delivery system of the present disclosure comprises a T cell targeting molecule designated MX1505 which comprises a first polypeptide of SEQ ID NO: 2 and a second polypeptide of SEQ ID NO: 20. In some aspects, the T cell targeted delivery system of the present disclosure comprises a T cell targeting molecule designated MX1517 which comprises a first polypeptide of SEQ ID NO: 3 and a second polypeptide of SEQ ID NO: 21. In some aspects, the T cell targeted delivery system of the present disclosure comprises a T cell targeting molecule designated MX1518 which comprises a first polypeptide of SEQ ID NO: 4 and a second polypeptide of SEQ ID NO: 22. In some aspects, the T cell targeted delivery system of the present disclosure comprises a T cell targeting molecule designated MX1507 which comprises a first polypeptide of SEQ ID NO: 5, a second polypeptide of SEQ ID NO: 23, and a third polypeptide of SEQ ID NO: 37. In some aspects, the T cell targeted delivery system of the present disclosure comprises a T cell targeting molecule designated MX1519 which comprises a first polypeptide of SEQ ID NO: 6, a second polypeptide of SEQ ID NO: 24, and a third polypeptide of SEQ ID NO: 38. In some aspects, the T cell targeted delivery system of the present disclosure comprises a T cell targeting molecule designated MX1506 which comprises a first polypeptide of SEQ ID NO: 7, a second polypeptide of SEQ ID NO: 25, and a third polypeptide of SEQ ID NO: 39. In some aspects, the T cell targeted delivery system of the present disclosure comprises a T cell targeting molecule designated MX1500 which comprises a first polypeptide of SEQ ID NO: 8 and a second polypeptide of SEQ ID NO: 26. In some aspects, the T cell targeted delivery system of the present disclosure comprises a T cell targeting molecule designated MX1502 which comprises a first polypeptide of SEQ ID NO: 9 and a second polypeptide of SEQ ID NO: 27. In some aspects, the T cell targeted delivery system of the present disclosure comprises a T cell targeting molecule designated MX1503 which comprises a first polypeptide of SEQ ID NO:10 and a second polypeptide of SEQ ID NO: 28. In some aspects, the T cell targeted delivery system of the present disclosure comprises a T cell targeting molecule designated MX1504 which comprises a first polypeptide of SEQ ID NO: 11 and a second polypeptide of SEQ ID NO: 29. In some aspects, the T cell targeted delivery system of the present disclosure comprises a T cell targeting molecule designated MX1505 which comprises a first polypeptide of SEQ ID NO: 12 and a second polypeptide of SEQ ID NO: 30. In some aspects, the T cell targeted delivery system of the present disclosure comprises a T cell targeting molecule designated MX1516 which comprises a first polypeptide of SEQ ID NO: 13 and a second polypeptide of SEQ ID NO: 31. In some aspects, the T cell targeted delivery system of the present disclosure comprises a T cell targeting molecule designated MX1520 which comprises a first polypeptide of SEQ ID NO: 14 and a second polypeptide of SEQ ID NO: 32. In some aspects, the T cell targeted delivery system of the present disclosure comprises a T cell targeting molecule designated MX1532 which comprises a first polypeptide of SEQ ID NO: 15 and a second polypeptide of SEQ ID of NO: 33. In some aspects, the T cell targeted delivery system of the present disclosure comprises a T cell targeting molecule designated MX1533 which comprises a first polypeptide of SEQ ID NO: 16 and a second polypeptide of SEQ ID NO: 34. In some aspects, the T cell targeted delivery system of the present disclosure comprises a T cell targeting molecule designated MX1542 which comprises a first polypeptide of SEQ ID NO: 17 and a second polypeptide of SEQ ID NO: 35. In some aspects, the T cell targeted delivery system of the present disclosure comprises a T cell targeting molecule designated MX1243 which comprises a first polypeptide of SEQ ID NO: 18 and second polypeptide of SEQ ID NO: 36.

In some aspects, the T cell targeted delivery system of the present disclosure comprises a T cell targeting molecule designated MX1501 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 1, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 19.

In some aspects, the T cell targeted delivery system of the present disclosure comprises a T cell targeting molecule designated MX1505 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or 100% sequence identity to SEQ ID NO: 2, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 20.

In some aspects, the T cell targeted delivery system of the present disclosure comprises a T cell targeting molecule designated MX1517 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 3, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 21.

In some aspects, the T cell targeted delivery system of the present disclosure comprises a T cell targeting molecule designated MX1518 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 4, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 22.

In some aspects, the T cell targeted delivery system of the present disclosure comprises a T cell targeting molecule designated MX1507 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 5, a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 23, and a third polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 37.

In some aspects, the T cell targeted delivery system of the present disclosure comprises a T cell targeting molecule designated MX1519 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% identity to SEQ ID NO: 6, a second polypeptide having at least about 80% identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 24, and a third polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% identity to SEQ ID NO: 38.

In some aspects, the T cell targeted delivery system of the present disclosure comprises a T cell targeting molecule designated MX1506 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or 100% sequence identity to SEQ ID NO: 7, a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 25, and a third polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 39.

In some aspects, the T cell targeted delivery system of the present disclosure comprises a T cell targeting molecule designated MX1500 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 8, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about sequence 99% identity, or about 100% identity to SEQ ID NO: 26.

In some aspects, the T cell targeted delivery system of the present disclosure comprises a T cell targeting molecule designated MX1502 or a variant thereof which comprises a first polypeptide having at least about 80% identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 9, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 27.

In some aspects, the T cell targeted delivery system of the present disclosure comprises a T cell targeting molecule designated MX1503 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 10, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 28.

In some aspects, the T cell targeted delivery system of the present disclosure comprises a T cell targeting molecule designated MX1504 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 11, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 29.

In some aspects, the T cell targeted delivery system of the present disclosure comprises a T cell targeting molecule designated MX1505 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or 100% sequence identity to SEQ ID NO: 12, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 30.

In some aspects, the T cell targeted delivery system of the present disclosure comprises a T cell targeting molecule designated MX1516 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 13, and a second polypeptide having at least about 80% identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 31.

In some aspects, the T cell targeted delivery system of the present disclosure comprises a T cell targeting molecule designated MX1520 or a variant thereof which comprises a first polypeptide having at least about 80% sequence identity, at least about 85% subsequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or 100% sequence identity to SEQ ID NO: 14, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 32.

In some aspects, the T cell targeted delivery system of the present disclosure comprises a T cell targeting molecule designated MX1532 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 15, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 33.

In some aspects, the T cell targeted delivery system of the present disclosure comprises a T cell targeting molecule designated MX1533 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 16, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 34.

In some aspects, the T cell targeted delivery system of the present disclosure comprises a T cell targeting molecule designated MX1542 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 17, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 35.

In some aspects, the T cell targeted delivery system of the present disclosure comprises a T cell targeting molecule designated MX1243 or a variant thereof which comprises a first polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about sequence 97% identity, at least about sequence 98% identity, at least about sequence 99% identity, or about 100% sequence identity to SEQ ID NO: 18, and a second polypeptide having at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or about 100% sequence identity to SEQ ID NO: 36.

In some aspects, the surface anchored T cell targeting molecule that specifically binds to at least two T cell specific surface proteins comprises a bispecific anti-CD3/anti-CD28 antibody selected from the group consisting of MX1243, MX1501, MX1505, MX1517, MX1518, MX1500, MX1502, MX1505, MX1516 and MX1520. In some aspects, the T cell targeted delivery system comprises at least two surface anchored T cell targeting molecules that are selected from the group consisting of MX1243, MX1501, MX1505, MX1517, MX1518, MX1500, MX1502, MX1505, MX1516, MX1520, MX1507, MX1519, MX1532, MX1506, MX1503, MX1533, MX1542, and MX1504, wherein at least one of the surface anchored T cell targeting molecules binds to CD3 and at least one of the surface anchored T cell targeting molecules bind to CD28. In some aspects, the first T cell targeting molecule that specifically binds to a first T cell specific surface protein (e.g., a first antibody targeting CD3), and the second T cell targeting molecule that specifically binds to a second T cell specific surface protein (e.g., a second antibody targeting CD28) in a set of LNP (e.g., two LNPs) are selected from the group consisting of MX1243, MX1501, MX1505, MX1517, MX1518, MX1500, MX1502, MX1505, MX1516, MX1520, MX1507, MX1519, MX1532, MX1506, MX1503, MX1533, MX1542, and MX1504. In some aspects, the T cell targeted delivery system comprises an anti-CD3/anti-CD28 bispecific antibody selected from the group consisting of MX1243, MX1501, MX1505, MX1517, MX1518, MX1500, MX1502, MX1505, MX1516, and MX1520. In some aspects, one or more of the anti-CD3 and/or anti-CD28 is attached to a LNP via a maleimide moiety. In some aspects, the T cell targeted delivery system comprises a LNP having MX1243 covalently attached to the surface of the LNP, e.g., via a maleimide moiety. In some aspects, the T cell targeted delivery system comprises an anti-CD3 antibody selected from the group consisting of MX1507, MX1519, MX1532, MX1533, and MX1542, and an anti-CD28 antibody selected from the group consisting of MX1506, MX1503, and MX1504. In some aspects, the T cell targeted delivery system comprises a LNP having an anti-CD3 antibody selected from the group consisting of MX1507, MX1519, MX1532, MX1533, and MX1542, and an anti-CD28 antibody selected from the group consisting of MX1506, MX1503, and MX1504. In some aspects, the T cell targeted delivery system comprises a LNP having one or more than one anti-CD3 antibodies selected from the group consisting of MX1507, MX1519, MX1532, MX1533, MX1542 and/or one or more than one anti-CD28 antibodies selected from the group consisting of MX1506, MX1503, and MX1504. In some aspects, the T cell targeted delivery system comprises a first LNP having an anti-CD3 antibody selected from the group consisting of MX1507, MX1519, MX1532, MX1533, and MX1542, and a second LNP having an anti-CD28 antibody selected from the group consisting of MX1506, MX1503, and MX1504. In some aspects, the T cell targeted delivery system comprises at least one antibody selected from the group consisting of MX1243, MX1501, MX1505, MX1517, MX1518, MX1500, MX1502, MX1505, MX1516, MX1520, MX1507, MX1519, MX1532, MX1506, MX1503, MX1533, MX1542, and MX1504. In some aspects, the T cell targeted delivery system comprises MX1506 and MX1507. In some aspects, MX1506 and MX1507 are in a single LNP. In some aspects, MX1506 and MX1507 are in separate LNPs.

In some aspects, the T cell targeted delivery system comprises a anti-CD3/anti-CD28 bispecific antibody, wherein the bispecific antibody is MX1243 (SEQ ID NO: 18/SEQ ID NO: 36), wherein the bispecific antibody is covalently attached to the surface of a LNP via a maleimide moiety, and wherein the LNP contains cholesterol (46.5 mol %), DSPE-PEG-2000-maleimide (0.5 mol %), DOPE (16% mol %), a cationic or ionizable cationic lipid or lipidoid selected from the group consisting of cKK-E12, SSOP, ALC-0315, Lipid 8, Lipid 10, SM-102 and combinations thereof (35% mol %) and DMG-PEG2000 (2% mol %). In some aspects, the T cell targeted delivery system further comprises an mRNA encapsulated in the LNP. In some aspects, the mRNA encodes a CAR.

In some aspects, the T cell targeted delivery system comprises a single population of LNP comprising a bispecific anti-CD3/anti-CD28 antibody (e.g., MX1243). In some aspects, the T cell targeted delivery system comprises a single population of LNP comprising a bivalent anti-CD antibody (e.g., MX1506). In some aspects, the T cell targeted delivery system comprises a single population of LNP comprising a bivalent anti-CD28 antibody (e.g., MX1507). In some aspects, the T cell targeted delivery system comprises a single population of LNP comprising a bivalent anti-CD3 antibody (e.g., MX1506) and a bivalent anti-CD28 antibody (e.g., MX1507). In some aspects, the T cell targeted delivery system comprises a single population of LNP comprising a bivalent anti-CD3 antibody (e.g., MX1506) and a bivalent anti-CD28 antibody (e.g., MX1507), wherein the percentages of anti-CD3 antibody and anti-CD28 antibody are 75% and 25%, respectively. In some aspects, the T cell targeted delivery system comprises a single population of LNP comprising a bivalent anti-CD3 antibody (e.g., MX1506) and a bivalent anti-CD28 antibody (e.g., MX1507), wherein the percentages of anti-CD3 antibody and anti-CD28 antibody are 50% and 50%, respectively. In some aspects, the T cell targeted delivery system comprises a single population of LNP comprising a bivalent anti-CD3 antibody (e.g., MX1506) and a bivalent anti-CD28 antibody (e.g., MX1507), wherein the percentages of anti-CD3 antibody and anti-CD28 antibody are 25% and 75%, respectively.

In some aspects, the T cell targeted delivery system comprises two populations of LNP, wherein the first LNP population comprises a bivalent anti-CD3 antibody (e.g., MX1506) and the second LNP population comprises a bivalent anti-CD28 antibody (e.g., 1507). In some aspects, the T cell targeted delivery system comprises two populations of LNP, wherein the first population comprises a bivalent anti-CD3 antibody (e.g., MX1506) and the second population comprises a bivalent anti-CD28 antibody (e.g., 1507), wherein the first population comprises 75% of the LNP and the second population comprises 25% of the LNP. In some aspects, the T cell targeted delivery system comprises two populations of LNP, wherein the first population comprises a bivalent anti-CD3 antibody (e.g., MX1506) and the second population comprises a bivalent anti-CD28 antibody (e.g., 1507), wherein the first population comprises 50% of the LNP and the second population comprises 50% of the LNP. In some aspects, the T cell targeted delivery system comprises two populations of LNP, wherein the first population comprises a bivalent anti-CD3 antibody (e.g., MX1506) and the second population comprises a bivalent anti-CD28 antibody (e.g., 1507), wherein the first population comprises 25% of the LNP and the second population comprises 75% of the LNP.

In some aspects, the efficacy of mRNA delivery to a T cell by a T cell targeted delivery system disclosed herein is at least about 25%, at least about 30%, at least about 35%, at least about 40%, at least about 45%, at least about 50%, at least about 55%, at least about 60%, at least about 65%, or at least about 70%. In some aspects, the efficacy of mRNA delivery to a T cell by a T cell targeted delivery system disclosed herein is about 25%, about 30%, about 35%, about 40%, about 45%, about 50%, about 55%, about 60%, about 65%, or about 70%. In some aspects, the efficacy of mRNA delivery to a T cell by a T cell targeted delivery system disclosed herein is at least about 25%, at least about 30%, at least about 35%, at least about 40%, at least about 45%, at least about 50%, at least about 55%, at least about 60%, at least about 65%, or at least about 70%. In some aspects, the efficacy of mRNA delivery to a T cell by a T cell targeted delivery system disclosed herein is about 25%, about 30%, about 35%, about 40%, about 45%, about 50%, about 55%, about 60%, about 65%, or about 70%.

In some aspects, the administration of T cell targeted delivery system disclosed herein results in anti-apoptotic protein Bcl-xL up-regulation by at least about 35%, at least about 40%, at least about 45%, at least about 50%, at least about 55%, at least about 60%, at least about 65%, at least about 70%, or at least about 75%. In some aspects, the administration of T cell targeted delivery system disclosed herein results in anti-apoptotic protein Bcl-xL up-regulation by about 35%, about 40%, about 45%, about 50%, about 55%, about 60%, about 65%, about 70%, or about 75%. In some aspects, the administration of T cell targeted delivery system disclosed herein results in T cell activation (Cd25 up-regulation) by at least about 30%, at least about 35%, at least about 40%, at least about 45%, at least about 50%, at least about 55%, at least about 60%, at least about 65%, at least about 70%, at least about 75%, or at least about 80%. In some aspects, the administration of T cell targeted delivery system disclosed herein results in T cell activation (Cd25 up-regulation) by about 30%, about 35%, about 40%, about 45%, about 50%, about 55%, about 60%, about 65%, about 70%, about 75%, or about 80%.

In some aspects, the T cell targeted delivery system comprises a pair of monospecific antibodies, for example MX1506 (bivalent anti-CD3) and MX1507 (bivalent anti-CD28), or a bispecific antibody, for example, MX1243 (bispecific anti-CD3/anti-CD28), wherein the antibodies are covalently attached to the surface of a LNP via a maleimide moiety, and wherein the LNP contains cholesterol (46.5 mol %), DSPE-PEG-2000-maleimide (0.5 mol %), DOPE (16% mol %), cKK-E12 (35% mol %) and DMG-PEG2000 (2% mol %). In some aspects, the T cell targeted delivery system comprises a pair of monospecific antibodies, for example MX1506 (bivalent anti-CD3) and MX1507 (bivalent anti-CD28), or a bispecific antibody, for example, MX1243 (bispecific anti-CD3/anti-CD28), wherein the antibodies are covalently attached to the surface of a LNP via a maleimide moiety, and wherein the LNP contains cholesterol (46.5 mol %), DSPE-PEG-2000-maleimide (0.5 mol %), DOPE (16% mol %), SSOP (35% mol %) and DMG-PEG2000 (2% mol %). In some aspects, the T cell targeted delivery system comprises a pair of monospecific antibodies, for example MX1506 (bivalent anti-CD3) and MX1507 (bivalent anti-CD28), or a bispecific antibody, for example, MX1243 (bispecific anti-CD3/anti-CD28), wherein the antibodies are covalently attached to the surface of a LNP via a maleimide moiety, and wherein the LNP contains cholesterol (46.5 mol %), DSPE-PEG-2000-maleimide (0.5 mol %), DOPE (16% mol %), ALC-0315 (35% mol %) and DMG-PEG2000 (2% mol %). In some aspects, the T cell targeted delivery system comprises a pair of monospecific antibodies, for example MX1506 (bivalent anti-CD3) and MX1507 (bivalent anti-CD28), or a bispecific antibody, for example, MX1243 (bispecific anti-CD3/anti-CD28), wherein the antibodies are covalently attached to the surface of a LNP via a maleimide moiety, and wherein the LNP contains cholesterol (46.5 mol %), DSPE-PEG-2000-maleimide (0.5 mol %), DOPE (16% mol %), SM-102 (35% mol %) and DMG-PEG2000 (2% mol %).

When MX1243 was conjugated to different LNPs, recovery of encapsulated mRNA following antibody conjugation to the LNPs was higher for LNPs comprising SM-102 (6.6%) and ALC-0315 (2.7%) than for LNPs comprising cKK-E12 (1.3%). The highest antibody content (μg/mL) was observed in LNPs comprising SM-102 (37 μg/mL), ALC-0315 (22.3 μg/mL), and SSOP (10.3 μg/mL) compared to LNPs comprising cKK-E12 (0.7 μg/mL). The highest conjugation efficiency with respect to recovery ratio (conjugation efficiency/recovery %) was observed with SM-102 LNPs and the lowest with cKK-E12 LNPs. Thus, it was wholly unexpected that LNPs containing cKK-E12 achieved the highest mRNA delivery among the different LNPs tested, and achieved T cell activation values comparable to the other LNPs tested.

### I.C.iv Bioavailability and Biodistribution Modifying Agents

In some aspects, a T cell targeted delivery system of the present disclosure comprises a bio-distribution modifying agent. As used herein, the term a “bio-distribution modifying agent,” which refers to an agent (i.e., payload) that can modify the distribution of extracellular vesicles (e.g., exosomes, nanovesicles) in vivo or in vitro (e.g., in a mixed culture of cells of different varieties).

In some aspects, the term “targeting moiety” can be used interchangeably with the term bio-distribution modifying agent. In some aspects, the targeting moiety alters the tropism of the LNP (“tropism moiety”). As used herein, the term “tropism moiety” refers to a targeting moiety that when expressed on an LNP alters and/or enhances the natural movement of the EV. In that respect, bispecific antibody covalently attached to an LNP of the present disclosure that targets CD3 and CD28 and therefore directs the LNP to T cells can be considered both a targeting moiety and a tropism moiety. Thus unless indicated otherwise, the term “targeting moiety” or “targeting molecule” as used herein, encompasses tropism moieties. In some aspects, the LNP comprises a targeting moiety, i.e., a biologically active molecule directing an LNP of the present disclosure to a T cell comprising an antibody and/or or a protein or ligand that interact with a protein or receptor on the surface of the T cell. Pharmacokinetics, biodistribution, and in particular tropism and retention in the desired tissue or anatomical location also can be accomplish by selecting the appropriate administration route (e.g., intrathecal administration or intraocular administration to improve tropism to the central nervous system).

In principle, the LNP of the present disclosure comprising at least one tropism moiety that can direct the LNP to a specific target cell or tissue (e.g., T cells) can be administered using any suitable administration method known in the art (e.g., intravenous injection or infusion) since the presence of the tropism moiety (alone or in combination with the presence of an antiphagocytic signal and the use of a specific administration route) will induce a tropism of the LNP towards the desired target cell or tissue.

In some aspects, the T cell targeted delivery system comprises a surface ligand covalently attached to the surface of a LNP of the present disclosure wherein the surface ligand can increase permeation through the blood-brain barrier. In some aspects, the surface ligand is a transferrin receptor. In some aspects, the T cell targeted delivery system comprises a surface ligand covalently attached to the surface of a LNP of the present disclosure wherein the surface ligand is a tissue or cell-specific target ligand that increases LNP tropism to a tissue or physiological compartment. In some aspects, the T cell targeted delivery system comprises a surface ligand covalently attached to the surface of a LNP of the present disclosure wherein the surface ligand is a surface anchored anti-phagocytic signal. In some aspects, the anti-phagocytic signal comprises CD47, CD24, a fragment thereof, and any combination thereof.

### II. Vectors and Cells

Methods that are well known to those skilled in the art can be used to construct vectors encoding antigen binding polypeptides and polypeptide complexes (e.g., CDR, VH, VL, heavy chain and/or light chain coding sequences and appropriate transcriptional and translational control signals). These methods include, for example, in vitro recombinant DNA techniques, synthetic techniques, and in vivo genetic recombination.

A vector can be transferred to a host cell by conventional techniques and the resulting cell can then be cultured by conventional techniques to produce an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain). Thus, provided herein are host cells containing a polynucleotide encoding an antigen binding polypeptide or polypeptide complex comprising, e.g., comprising six CDRs, VH, VL, VH and VL, heavy chain, light chain, or heavy and light chain, or a domain thereof (e.g., one or more CDRs, VH, VL, VH and VL, heavy chain, or light chain), operably linked to a promoter for expression of such sequences in the host cell. In some aspects, vectors encoding both heavy and light chains, or a domain thereof, individually, can be co-expressed in the host cell for expression. In some aspects, a host cell contains a vector comprising a polynucleotide encoding both a heavy chain and light chain, or a domain thereof. In some aspects, a host cell contains two different vectors, a first vector comprising a polynucleotide encoding a heavy chain or a domain thereof, and a second vector comprising a polynucleotide encoding a light chain or a domain thereof. In some aspects, a first host cell comprises a first vector comprising a polynucleotide encoding a heavy chain or a domain thereof, and a second host cell comprises a second vector comprising a polynucleotide encoding a light chain or a domain thereof. In some aspects, the present disclosure provides a population of host cells comprising such a first host cell and such a second host cell.

In some aspects, provided herein is a population of vectors comprising a first vector comprising a polynucleotide encoding a light chain or domain thereof, and a second vector comprising a polynucleotide encoding a heavy chain or domain thereof. Alternatively, a single vector can be used which encodes, and is capable of expressing, both heavy and light chain polypeptides or a domain thereof.

A variety of host-vector systems can be utilized to express the polypeptides and polypeptide complexes provided herein. Such host-vector systems represent vehicles by which the coding sequences of interest can be produced and subsequently purified, but also represent cells which can, when transformed or transfected with the appropriate nucleotide coding sequences, express a polypeptide or polypeptide complex provided herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS 1 or COS), CHO, BHK, MDCK, HEK 293, NS0, PER.C₆, VERO, CRL7O3O, HsS78Bst, HeLa, and NIH 3T3, HEK-293T, HepG2, SP210, R1.1, B—W, L-M, BSC1, BSC40, YB/20, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing polypeptide or polypeptide complexes described herein are CHO cells, for example CHO cells from the CHO GS System™ (Lonza). In some aspects, cells for expressing the polypeptides or polypeptide complexes provided herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking & Hofstetter (1986) Gene 45: 101-105; and Cockett et al., (1990) Biotechnol. 8: 662-667). In some aspects, the polypeptides or polypeptide complexes provided herein are produced by HEK-293 T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells that possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7O3O, COS (e.g., COS 1 or COS), PER.C₆, VERO, HsS78Bst, HEK-293T, HepG2, SP210, R1.1, B—W, L-M, BSC1, BSC40, YB/20, BMT10 and HsS78Bst cells.

Once a polypeptide or polypeptide complex provided herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes provided herein can be fused to heterologous polypeptide sequences provided herein (e.g., peptide tags) or otherwise known in the art to facilitate purification.

In some aspects, a polypeptide or polypeptide complex provided herein is isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex described herein is substantially free of cellular material and/or chemical precursors.

In some aspects, specific constructs disclosed herein can be generated recombinantly using the coding polynucleotide sequences disclosed in TABLE 10.

TABLE 10
Sequence encoding bispecific antibodies.

| Bispecific | Polypeptide 1 | Polypeptide 2 | Polypeptide 3 |
|---|---|---|---|
| antibody | HC - knob chain | HC- hole chain | LC |
| MX1501 | 64 | 82 | |
| MX1505 | 65 | 83 | |
| MX1517 | 66 | 84 | |
| MX1518 | 67 | 85 | |
| MX1507 | 68 | 86 | 100 |
| MX1519 | 69 | 87 | 101 |
| MX1506 | 70 | 88 | 102 |
| MX1500 | 71 | 89 | |
| MX1502 | 72 | 90 | |
| MX1503 | 73 | 91 | |
| MX1504 | 74 | 92 | |
| MX1505 | 75 | 93 | |
| MX1516 | 76 | 94 | |
| MX1520 | 77 | 95 | |
| MX1532 | 78 | 96 | |
| MX1533 | 79 | 97 | |
| MX1542 | 80 | 98 | |
| MX1243 | 81 | 99 | |

### III. Method of Manufacturing

The present disclosure provides methods of manufacturing a T cell targeted delivery system comprising conjugating a T cell targeting antibody or combination thereof to a lipid via a maleimide moiety, wherein the lipid integrates into the external surface of the LNP. In some aspects, the T cell targeting antibody is a monospecific antibody or a combination thereof (e.g., a first monospecific antibody targeting CD3 and a second monospecific antibody targeting CD28). In some aspects, the T cell targeting antibody is a bispecific antibody, e.g., an anti CD3/anti CD28 bispecific antibody.

In some aspects, a first monospecific T cell targeting antibody is conjugated to a first lipid and a second monospecific T cell targeting antibody is conjugated to a second lipid, thereby yielding a first conjugate and a second conjugate, wherein the first conjugate integrates into the external surface of a first LNP and the second conjugate integrates into the external surface of a second LNP. In some aspects, the method further comprises combining the first LNP and the second LNP after the conjugation of the first T cell targeting antibody to the surface of a first LNP and the conjugation of the second T cell targeting antibody to the surface of the second LNP.

In some aspects, the conjugation comprises reacting a maleimide group covalently attached to the lipid with a sulfhydryl group of the T cell targeting antibody. In some aspects, the lipid is DSPE-PEG2000-maleimide, DMG-PEG2000-maleimide, cholesterol-PEG2000-maleimide, DSPE-PEG5000-maleimide, DMG-PEG5000-maleimide, cholesterol-PEG5000-maleimide or a combination thereof.

In some aspects, the conjugation comprises reacting a maleimide group attached to the T cell targeting antibody with a sulfhydryl group covalently attached to the lipid. In some aspects, the lipid is DSPE-PEG2000-SH, DMG-PEG2000-SH, cholesterol-PEG2000-SH, DSPE-PEG5000-SH, DMG-PEG5000-SH, cholesterol-PEG5000-SH or a combination thereof. In some aspects, the antibody-[maleimide]-lipid conjugate is formed on the LNP. In some aspects, the antibody-[maleimide]-lipid conjugate is generated prior to the assembly of the LNP used as a component in the assembly of the LNP. In some aspects, the antibody-[maleimide]-lipid conjugate is generated independently and incorporated to a previously assembled LNP.

The maleimide group reacts specifically with sulfhydryl groups when the pH of the reaction mixture is between 6.5 and 7.5; the result is formation of a stable thioether linkage that is not reversible (i.e., the bond cannot be cleaved with reducing agents). In more alkaline conditions (pH>8.5), the reaction favors primary amines and increases the rate of hydrolysis of the maleimide group to a non-reactive maleamic acid. Maleimides do not react with tyrosines, histidines or methionines. Thiol-containing compounds, such as dithiothreitol (DTT) and beta-mercaptoethanol (BME), must be excluded from reaction buffers used with maleimides because they will compete for coupling sites. If DTT were used to reduce disulfides in a protein to make sulfhydryl groups available for conjugation, the DTT would have to be thoroughly removed using a desalting column before initiating the maleimide reaction. The disulfide-reducing agent TCEP does not contain thiols and does not have to be removed before reactions involving maleimide reagents. Excess maleimides can be quenched at the end of a reaction by adding free thiols. EDTA can be included in the coupling buffer to chelate stray divalent metals that otherwise promote oxidation of sulfhydryls (non-reactive).

In some aspects, the conjugation process comprises treating the T cell targeting molecule, e.g., an antibody, with a reducing agent. Suitable reducing agents include, for example, TCEP (Tris(2-carboxyethyl)phosphine), DTT (dithiothreitol), BME (2-mercaptoethanol), a thiolating agent, and any combination thereof. The thiolating agent can comprise, e.g., Traut's reagent (2-iminothiolane). In some aspects, an antibody of the present disclosure is treated with DTT at 37° C. for 1 hour. After the treatment with the reducing agent, the linking reaction further comprises bringing the T cell targeting molecule, e.g., an antibody, in contact with the maleimide reactive groups present on the surface of the LNP (e.g., in DSPE-PEG2000-maleimide). In some aspects, the maleimide group is reacted with the T cell targeting molecule, e.g., an antibody, prior to the incorporation of the conjugate in LNP. In some aspects, the maleimide moiety is further attached to a linker/spacer to connect the maleimide moiety to the T cell targeting molecule, e.g., an antibody. In some aspects, the spacer is PEG2000 interposed between the maleimide group/moiety and the lipid (e.g., DSPE).

The methods of manufacture disclosed herein also comprise the preparation of a LNP, which can be derivatized as described above by conjugating, e.g., a T cell targeting molecule such as an antibody, to the surface of the LNP via a maleimide moiety. Exemplary LNP compositions and methods for making them are described, for example, herein and in U.S. Pat. Nos. 11,524,023; 11,485,972; 10,898,574; 10,703,789; 10,702,600; 10,577,403; 10,442,756; 10,266,485; 10,064,959; 9,868,692; Int'l Pub. No. WO 2019/046809; Int'l Pub. No. WO 2020/160397; U.S. Pub. No. 2020/0306191; Xu et al., Adv. Nanobio. Res. 2:2, 2022; Cullis et al., Mol. Ther. 25(7):1467-1475, 2017; Fan et al., J. Pharm. Biomed. Anal. 192:113642, 2020; Paunovska et al., Nature Rev., 23:265-280, 2022; Buck et al., ACS Nano. 13:3754-3782, 2019; Pardi et al., Nature Comm. 8:14630, 2017; and Pardi et al., J. Control Release, 217:345-351, 2015, which are incorporated by reference herein. Such methods include, but are not limited to, back translating DNA coding for an antigen binding polypeptide or antigen binding polypeptide complex provided herein (e.g., an antibody or antigen binding fragment thereof) into an in vitro transcription (IVT) plasmid according to such published methods.

The incorporation of T cell targeting molecules (e.g., antibodies) to the surface of a LNP can take place by mixing two populations of T cell targeting molecules prior to their conjugation to the surface of the LNP. In some aspects, the T cell targeting molecules (e.g., antibodies) are conjugated sequentially to the surface of the LNP. Thus, for example, a first antibody could be conjugated to the surface of the LNP, and after the first conjugation reaction a second conjugation reaction attaching the second antibody to the surface of the LNP would take place. In some aspects, different T cell targeting molecules (e.g., antibodies) would be conjugated to the surfaces of different populations of LNPs (which could have the same lipid composition or different lipid compositions) and the populations of LNPs would be subsequently combined (mixed).

The incorporation of T cell targeting molecules (e.g., antibodies) to the surface of a LNP can take place using a conjugation approach know in the art. See, e.g., Adhikari P et al. Antibody-Drug Conjugates, Methods and Protocols. Methods Mol. Biol. 2019; 2078:51-69; and McPherson M J, Hobson A D. Antibody-Drug Conjugates, Methods and Protocols. Methods Mol. Biol. 2019; 2078:23-36, which are incorporated by reference in their entireties.

### IV. Pharmaceutical Composition

The present disclosure provides pharmaceutical compositions comprising a therapeutic agent encapsulated in a T cell targeted delivery system disclosed herein. In some aspects, the pharmaceutical composition comprises a pharmaceutically acceptable carrier. In some aspects, the present disclosure provides a pharmaceutical composition comprising a T cell targeting molecule (antigen binding polypeptide, antigen binding polypeptide complex, e.g., antibody or antigen binding fragment thereof), polypeptide, polynucleotide, vector, or host cell provided herein. In some aspects, the pharmaceutical composition is encapsulated in a LNP.

In some aspects, a pharmaceutical composition provided herein comprises (1) a LNP comprising a bispecific T cell targeting molecule (antigen binding polypeptide, antigen binding polypeptide complex, e.g., antibody or antigen binding fragment thereof) provided herein; and, (2) a pharmaceutically acceptable carrier. In some aspects, a payload (e.g., an mRNA encoding a CAR) is encapsulated in the LNP. In some aspects, a pharmaceutical composition provided herein comprises (1) a LNP comprising two monospecific T cell targeting molecules (antigen binding polypeptide, antigen binding polypeptide complex, e.g., antibody or antigen binding fragment thereof) provided herein; and (2) a pharmaceutically acceptable carrier. In some aspects, a payload (e.g., an mRNA encoding a CAR) is encapsulated in the LNP.

In some aspects, a pharmaceutical composition provided herein comprises (1) two populations of LNPs, each population of LNP comprising a monospecific T cell targeting molecule (antigen binding polypeptide, antigen binding polypeptide complex, e.g., antibody or antigen binding fragment thereof) provided herein; and (2) a pharmaceutically acceptable carrier. In some aspects, a payload (e.g., an mRNA encoding a CAR) or a combination thereof is encapsulated in the two populations of LNPs. In some aspects, the monospecific T cell targeting molecule is selected from the group consisting of MX1243, MX1501, MX1505, MX1517, MX1518, MX1500, MX1502, MX1505, MX1516, MX1520, MX1507, MX1519, MX1532, MX1506, MX1503, MX1533, MX1542, and MX1504. In some aspects, a pharmaceutical composition provided herein comprises (1) a LNP comprising MX1243 attached to the LNP surface via a maleimide moiety; and (2) a pharmaceutically acceptable carrier. In some aspects, an mRNA encoding a CAR is encapsulated in the LNP.

The term “pharmaceutically acceptable carrier” includes any solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions provided herein. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies. In some aspects, the pharmaceutical composition is for parenteral, intravenous or subcutaneous administration. Once a pharmaceutical composition has been formulated, it can be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. In some aspects, the present disclosure provides a diagnostic composition comprising a diagnostic agent (e.g., a radionuclide) attached to a T cell targeted delivery system of the present disclosure.

### V. Kits

The present disclosure provides a kit comprising (1) a T cell targeted delivery system or one or more of its components, a pharmaceutical composition disclosed herein, or a diagnostic composition disclosed herein, and (2) optionally instructions for use. In some aspects, the kit comprises (1) a T cell targeted delivery system of the present disclosure and (2) optionally instructions for encapsulation of a biologically active molecule, e.g., an mRNA. In some aspects, the kit comprises (1) a LNP or components thereof, and (2) instructions to encapsulate a biologically active molecule, e.g., an mRNA, in the LNP. In some aspects, the kit comprises (1) a biologically active molecule encapsulated in a LNP, (2) a T cell targeting molecule, e.g., an antibody selected from the group consisting of MX1243, MX1501, MX1505, MX1517, MX1518, MX1500, MX1502, MX1505, MX1516, MX1520, MX1507, MX1519, MX1532, MX1506, MX1503, MX1533, MX1542, and MX1504, (3) optionally instructions to conjugate the T cell targeting molecule to the LNP, and (4) optionally instructions to incorporate one of more biologically active molecules to the LNP. In some aspects, the kit comprises (1) reagents for the production of LNPs, (2) reagents for conjugating a T cell targeting molecule, e.g., an antibody selected from the group consisting of MX1243, MX1501, MX1505, MX1517, MX1518, MX1500, MX1502, MX1505, MX1516, MX1520, MX1507, MX1519, MX1532, MX1506, MX1503, MX1533, MX1542, and MX1504, to the LNP, (3) optionally instructions to conjugate the T cell targeting molecule to the LNPs, and (4) optionally instructions to incorporate one of more biologically active molecules to the LNP. In some aspects, the kit comprises (1) reagents for the production of two populations of LNPs, (2) reagents for conjugating a T cell targeting molecule, e.g., an antibody selected from the group consisting of MX1243, MX1501, MX1505, MX1517, MX1518, MX1500, MX1502, MX1505, MX1516, MX1520, MX1507, MX1519, MX1532, MX1506, MX1503, MX1533, MX1542, and MX1504, to the LNP, and (3) optional instructions to conjugate the T cell targeting molecule to the LNPs. In some aspects, the kit comprises (1) two populations of LNP, wherein each population comprises a different T cell targeting molecule, e.g., an antibody selected from the group consisting of MX1243, MX1501, MX1505, MX1517, MX1518, MX1500, MX1502, MX1505, MX1516, MX1520, MX1507, MX1519, MX1532, MX1506, MX1503, MX1533, MX1542, and MX1504, and (2) instructions to combine the two populations at specific ratios for administration to a subject in need thereof.

### VI. Methods of Use

The present disclosure provides a method of treating or preventing a disease or disorder in a subject in need thereof comprising administering to the subject a therapeutic agent or combination thereof encapsulated in a T cell targeted delivery system disclosed herein or attached (e.g., covalently attached) to a T cell targeted delivery system disclosed herein.

A gene therapy method comprising administering a therapeutic agent or combination thereof encapsulated in a T cell targeted delivery system disclosed herein or attached (e.g., covalently attached) to a T cell targeted delivery system disclosed herein. In some aspects, the gene therapy is in vivo. In some aspects, the gene therapy is ex vivo. In some aspects, the therapeutic agent comprises an mRNA, a vector, or a guide RNA (gRNA). In some aspects, the therapeutic agent comprises a nucleic acid (e.g., an antisense oligonucleotide, an mRNA, or gRNA), a protein, a small molecule, or a combination thereof. Numerous payloads that can be used, alone or in combination, in T cell targeted delivery systems disclosed herein and used therapeutically are disclosed in the present application.

The present disclosure also provides a CAR-T cell therapy method comprising administering a T cell targeted delivery system disclosed herein to the subject. In some aspects, the T cell targeted delivery system comprises a vector encoding a CAR protein. In some aspects, the T cell targeted delivery system comprises an mRNA encoding a CAR protein. In some aspects, the vector or mRNA are in the lumen of the LNP. In some aspects, the T cell targeted delivery system comprises a CAR protein, e.g., a CAR protein integrated in the LNP or covalently linked to the LNP. In some aspects, the CAR protein is in the lumen of the LNP. In some aspects, the CAR protein is integrated in the membrane of the LNP. The present disclosure provides a method to target at least one payload to a T cell comprising (i) encapsulating a payload in a T cell targeted delivery system disclosed herein; or, (ii) attaching a payload to a T cell targeted delivery system disclosed herein (e.g., via a cleavable linker); or, (iii) a combination thereof. In some aspects, a payload is attached to the inner surface of the LNP, attached to the outer surface or the LNP, transverses the lipid layer of the LNP, or, a combination thereof.

As used herein, the terms “prevent” or “preventing” refer to the prevention of the onset, recurrence or spread, in whole or in part, of a disease or condition provided herein, or a symptom thereof. As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, “administering” is meant a method of giving a dosage of a T cell targeted delivery system disclosed herein comprising one or more therapeutic agents (e.g., mRNA) to a subject in need thereof (e.g., a patient). Administering can be by any suitable means, including parenteral, intrapulmonary or intranasal. Parenteral infusions include, for example, intramuscular, intravenous, intraarterial, intraperitoneal or subcutaneous administration. Dosing can be by any suitable route, e.g., by injection, such as intravenous or subcutaneous injection. Various dosing schedules including, but not limited to, single or multiple administrations over various time-points, bolus administration, and pulse infusion are contemplated herein.

As used herein, a “therapeutically effective amount” is an amount of a T cell targeted delivery system disclosed herein comprising one or more therapeutic agents (e.g., mRNA) that is sufficient to achieve the desired effect and can vary according to the nature and severity of the condition, and the potency of the T cell targeted delivery system disclosed herein comprising one or more therapeutic agents (e.g., mRNA). A therapeutic effect is the relief, to at least some extent, of one or more symptoms of the disease or disorder, and can include curing a disease or disorder. “Curing,” “cure,” and grammatical variants thereof means that the symptoms of active disease are eliminated. However, certain long-term or permanent effects of a disease or disorder can exist even after a cure is obtained.

As used herein, the term “subject” means a human or a non-human mammal, e.g., a dog, cat, mouse, rat, cow, sheep, pig, goat, non-human primate or bird, e.g., chicken, as well as any other vertebrate or invertebrate. In some aspects, the subject is a human. In some aspects, the subject is a veterinary animal. In some aspects, the subject is a mammal.

In some aspects, the present disclosure provides a method of treating cancer. In some aspects, the cancer is a hematologic cancer. In some aspects, the hematologic cancer is a lymphoma, myeloma, or leukemia. In some aspects, the lymphoma is a Hodgkin lymphoma or Non-Hodgkin lymphoma. In some aspects, the myeloma is a multiple myeloma, solitary plasmocytoma, or extramedullary plasmocytoma. In some aspects, the leukemia is an Acute Lymphocytic Leukemia (ALL), Chronic Lymphocytic Leukemia (CLL), Acute Myeloid Leukemia (AML), Chronic Myeloid Leukemia (CML), Hairy Cell Leukemia, Childhood Acute Lymphoblastic Leukemia, or Childhood Acute Myeloid Leukemia. In some aspects, the cancer is a solid cancer. In some aspects, the solid cancer is a breast cancer, colon cancer, kidney cancer, lung cancer, liver cancer, skin cancer, brain cancer, bladder cancer, prostate cancer, endometrial cancer, pancreatic cancer, or thyroid cancer.

In some aspects, the present disclosure provides a method for treating an immune disease. In some aspects, the immune disease is artherosclerosis, Lyme disease, or fibromyalgia. In some aspects, the immune disease is an autoimmune disease. In some aspects, the autoimmune disease is Addison disease, rheumatoid arthritis, inflammatory bowel disease, celiac disease, Hashimoto thyroiditis, Graves disease, Myasthenia gravis, multiple sclerosis, dermatomyositis, systemic lupus erythematosus, type I diabetes, Sjögren syndrome, Giant cell arteritis, reactive arthritis, pernicious anemia, alopecia areata, ankylosing spondylitis, antiphospholipid syndrome, Guillain-Barre syndrome, or psoriasis.

In some aspects, the disease or disorder is a cancer, an inflammatory disease, a neurodegenerative disorder, a central nervous disease or a metabolic disease. In some aspects, a disease or disorder that can be treated with the present methods comprises a cancer, graft-versus-host disease (GvHD), autoimmune disease, infectious diseases, or fibrotic diseases. In some aspects, the disease or disorder is a cancer. When administered to a subject with a cancer, in some aspects, a composition of the present disclosure can up-regulate an immune response and enhance the tumor targeting of the subject's immune system.

In some aspects, the cancer being treated is characterized by infiltration of leukocytes (T cells, B-cells, macrophages, dendritic cells, monocytes) into the tumor microenvironment, or so-called “hot tumors” or “inflammatory tumors.” In some aspects, the cancer being treated is characterized by low levels or undetectable levels of leukocyte infiltration into the tumor microenvironment, or so-called “cold tumors” or “non-inflammatory tumors.” In some aspects, the composition of the present disclosure is administered in an amount and for a time sufficient to convert a “cold tumor” into a “hot tumor,” i.e., said administering results in the infiltration of leukocytes (such as T cells) into the tumor microenvironment.

In some aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. In some aspects, the terms “distal tumor” or “distant tumor” refer to a tumor that has spread from the original (or primary) tumor to distant organs or distant tissues, e.g., lymph nodes. In some aspects, the composition of the present disclosure can treat a tumor after the metastatic spread.

In some aspects, the disease or disorder is a graft-versus-host disease (GvHD). In some aspects, the disease or disorder that can be treated with the present disclosure is an autoimmune disease. Non-limiting examples of autoimmune diseases include: multiple sclerosis, peripheral neuritis, Sjogren's syndrome, rheumatoid arthritis, alopecia, autoimmune pancreatitis, Behcet's disease, bullous pemphigoid, celiac disease, Devic's disease (neuromyelitis optica), glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, and combinations thereof.

In some aspects, the disease or disorder is an infectious disease. In some aspects, the disease or disorder is an oncogenic virus. In some aspects, infectious diseases that can be treated with the present disclosure includes, but not limited to, human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV, SARS CoV, or SARS CoV2), filoviruses (e.g., Marburg and Ebola), Streptococcus pyogenes, Streptococcus pneumoniae, Plasmodia species (e.g., vivax and falciparum), Chikunga virus, human Papilloma virus (HPV), hepatitis B, hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, a disease or disorder that can be treated with the present methods comprises a Pompe disease, Gaucher, a lysosomal storage disorder, mucoviscidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, α1 antitrypsin deficiency, Retts Syndrome, Dravet Syndrome, Angelman Syndrome, DM1 disease, Fragile X disease, Huntingtons Disease, Friedreichs ataxia, CMT disease (also known as Charcot-Marie-Tooth disease, hereditary motor and sensory neuropathy (HMSN), or peroneal muscular atrophy), CMT1X disease, catecholaminergic polymorphic ventricular tachycardia, spinocerebellar ataxia type 3 (SCA3) disease, limb-girdle muscular dystrophy, or a hypercholesterolemia. In some aspects, the treatment is prophylactic.

In some aspects, the disease or disorder is a neurodegenerative disease. In some aspects, the neurodegenerative disease is selected from Alzheimer's disease, Parkinson's disease, prion disease, motor neuron disease, Huntington's disease, spinocerebellar ataxia, spinal muscular atrophy, and any combination thereof.

In some aspects, the disease or disorder comprises a muscular dystrophy. In some aspects, the muscular dystrophy is selected from Duchenne type muscular dystrophy (DMD), myotonic muscular dystrophy, facioscapulohumeral muscular dystrophy (FSHD), congenital muscular dystrophy, limb-girdle muscular dystrophy (including, but not limited to, LGMD2B, LGMD2D, LGNMD2L, LGMD2C, LGMD2E and LGMD2A), and any combination thereof.

In some aspects, the disease or disorder is selected from AADC deficiency (CNS), ADA-SCID, Alpha-1 antitrypsin deficiency, β-thalassemia (severe sickle cell), Cancer (head and neck squamous cell), Niemman-Pick Type C Disease, Cerebral ALD, Choroideremia, Congestive heart failure, Cystic Fibrosis, Duchenne muscular dystrophy (DMD), Fabry disease, Glaucoma, Glioma (cancer), Hemophilia A, Hemophilia B, HoFH (hypercholesterolemia), Huntington's Disease, Lipoprotein lipase deficiency, Leber hereditary optic neuropathy (LHON), Metachromatic leukodystrophy, MPS I (Hurler syndrome), MPS II (Hunter's syndrome), MPS III (Sanfilippo Syndrome), Parkinson's disease, Pompe Disease, Recessive Dystrophic Epidermolysis Bullosa, RPE65 deficiency (vision loss), Spinal Muscular Atrophy (SMA I), Wet AMD (retinal disease), Wiskott Aldrich syndrome (WAS), Mucopolysaccharidosis type IIIA (MPS IIIA), X-linked myotubular myopathy, X-linked retinitis pigmentosa, and any combination thereof.

### Optimization of CAR-T Cell Compositions and Methods

The CAR-T strategy disclosed in the present application is highly advantageous with respect to the traditional CAR-T methods. The traditional CAR-T approach requires a lengthy (4-12 weeks), costly ($375,000 to $475,000 per treatment), and patient specific process that comprises four discrete steps, namely, (i) leukapheresis, (ii) T cell activation and transduction, (iii) modified T cell expansion, and (iv) modified T cell infusion.

The traditional CAR-T cell approach requires lymphodepletion. In contrast, the CAR-T cell approach disclosed in the present application can be applied instantly, without requiring a 4-12 weeks delay, because the process only requires in vivo CAR-T cell infusion using an off-the-self, single product. This novel strategy does not require lymphodepletion and therefore is simpler and more cost-effective than traditional CAR-T cell technology.

In some aspects, the steps to implement the CAR-T methods of the present disclosure can be summarized as follows: (i) antibody selection (e.g., an anti-CD3/anti-CD28 bispecific antibody), (ii) LNP formulation, (iii) antibody conjugation (e.g., maleimide-based conjugation using a thiol group in the antibody), (iv) purification and quality control, and (v) LNP storage until use.

In some aspects, conjugated antibody targeting LNPs is required for efficient cargo delivery; thus, in some aspects, the CAR-T technology of the present disclosure relies on the use of antibodies targeting two receptors, e.g., CD28 and CD3, on the surface of T cells. In some aspects this targeting can be accomplished, e.g., by using two monospecific antibodies, a first one targeting CD28 (e.g., MX1506) and a second one targeting CD3 (e.g., MX1507 or MX1519) conjugated to the surface of LNPs. In some aspects, this targeting can be accomplished, e.g., by using bispecific antibodies targeting simultaneously two receptors, e.g., CD28 and CD3 (see, e.g., MX1500, MX1243, or MX1516) conjugated to the surface of LNPs. An advantageous property of the engineered LNPs of the present disclosure comprising antibodies targeting two receptors, e.g., CD3 and CD28, on the surface of T cells is that there is minimal delivery of the payload to the liver.

Lipid selection is a crucial aspect of the optimization process of the CAR-T methods of the present disclosure, in particular, selection of the appropriate ionizable cationic lipid. In some aspects, the ionizable cationic lipid is selected from the group consisting of ALC-315, cKK-E12, Syn3, MC3, KC2, Lipid 8 (see FIG. 31), Lipid 10 (see FIG. 31), and any one of MDX1-MDX13 (FIGS. 57-59).

In some aspects, the ionizable cationic lipid is Lipid 10 (see FIG. 31). Lipid 10 is a piperazine-containing lipid that represents a new class of ionizable cationic lipid that contains two ionizable groups compared to just one for a traditional ionizable cationic lipid. Thus, in some aspects, the LNPs for targeted delivery of the present disclosure comprise an ionizable cationic lipid comprising two ionizable groups, e.g., Lipid 10. In some aspects, the LNPs for targeted delivery of the present disclosure comprise an ionizable cationic lipid comprising an ionizable piperazine group. In some aspects, the ionizable cationic lipid is Lipid 8 (see FIG. 31).

The CAR-T methods of the present disclosure comprise an optimized antibody conjugation step. It is unexpected that reducing the fraction of maleimide-PEG used to conjugate the anti-CD3/anti-CD-28 to the surface of the LNP, and therefore the number of antibodies, significantly reduced T cell activation potential, but did not affect potency. In other words, reducing maleimide, e.g., to ¼ of the original amount (0.2 maleimide to 0.05 maleimide) resulted in the same gene delivery, albeit with reduced T cell activation.

The methods of the present disclosure are not limited to T cells. They are also applicable to other cell types such as B cells. For example, in some aspects, LNPs of the present disclosure are targeted to T cells, e.g., with anti-CD3/anti-CD28 conjugated to their surface, and the LNPs specifically deliver their payload (e.g., an mRNA encoding a CAR) to T cells. Conversely, in some aspects, LNPs of the present disclosure are targeted to B cells, e.g., with anti-CD19/anti-CD79/anti-CD40L conjugated to their surface, the LNPs specifically deliver their payload to B cells.

In some aspects, the LNPs of the present disclosure are prepared using a manufacturing process comprising the steps of (i) preparing the payload, e.g., an mRNA payload, (ii) preparing an LNP intermediate (LNP pre-conjugation) which comprises the encapsulating the payload in the LNP; (iii) preparing the antibody-conjugated LNP by covalently attaching targeting antibodies to the surface of the LNP intermediate, e.g., via a maleimide moiety, and (iv) formulating the drug product comprising the antibody-conjugated LNP containing the mRNA payload. This manufacturing workflow is summarized in FIG. 46.

In some aspects, the mRNA used as payload has at least about 80% purity. In some aspects, the mRNA used as payload has a purity of at least about 70%, at least about 75%, at least about 80%, at least about 85%, at least about 90%, at least about 95%, at least about 96%, at least about 97%, at least about 98%, at least about 99%. In some aspects, the mRNA use as payload has a purity of about 70%, about 75%, about 80%, about 85%, about 90%, about 95%, about 96%, about 97%, about 98%, or about 99%. In some aspects, the mRNA used as payload has a purity between about 70% and about 80%, between about 75% and 85%, between about 80% and about 90%, between about 85% and about 95%, between about 90% and about 100%, between about 85% and about 100%, or between about 80% and about 100%.

In some aspects, the mRNA used as payload has a 5′ cap efficiency of at least about 95%. In some aspects, the mRNA used as payload has a 5′ cap efficiency of at least about 95%, at least about 96%, at least about 97%, at least about 98%, or at least 99%. In some aspects, the mRNA used as payload has a 5′ cap efficiency of about 95%, about 96%, about 97%, about 98%, about 99% or about 100%.

In some aspects, the mRNA used as payload has a poly(A) tail with from about 100 nucleotides to about 120 nucleotides. In some aspects, the mRNA used as payload has a poly(A) tail of about 100 nucleotides, about 105 nucleotides, about 110 nucleotides, about 115 nucleotides, or about 120 nucleotides. In some aspects, the mRNA used as payload has a poly(A) tail of about 100 nucleotides to about 110 nucleotides or about 110 nucleotides to about 120 nucleotides. In some aspects, the mRNA used as payload has a poly(A) tail of 100 nucleotides, 101 nucleotides, 102 nucleotides, 103 nucleotides, 104 nucleotides, 105 nucleotides, 106 nucleotides, 107 nucleotides, 108 nucleotides, 109 nucleotides, 110 nucleotides, 111 nucleotides, 112 nucleotides, 113 nucleotides, 114 nucleotides, 115 nucleotides, 116 nucleotides, 117 nucleotides, 118 nucleotides, 119 nucleotides, or 120 nucleotides.

In some aspects, the mRNA payload stock prior to encapsulation in the LNP intermediate is at concentration of at least 50 μg/mL In some aspects, the mRNA payload stock prior to encapsulation in the LNP intermediate is at concentration of at least about 20 μg/mL, at least about g/mL, at least about 30 μg/mL, at least about 35 μg/mL, at least about 40 μg/mL, at least about g/mL, at least about 50 μg/mL, at least about 55 μg/mL, at least about 60 μg/mL, at least about 65 μg/mL, at least about 70 μg/mL, at least about 75 μg/mL, at least about 80 μg/mL, at least about 85 μg/mL, at least about 90 μg/mL, at least about 95 μg/mL, at least about 100 μg/mL. In some aspects, the mRNA payload stock prior to encapsulation in the LNP intermediate is at concentration of about 20 μg/mL, about 25 μg/mL, about 30 μg/mL, about 35 μg/mL, about 40 μg/mL, about 45 g/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, or about 100 μg/mL. In some aspects, the mRNA payload stock prior to encapsulation in the LNP intermediate is at concentration of about 20 to about 30 μg/mL, about 25 to about 35 μg/mL, about 30 to about 40 μg/mL, about 35 to about 45 μg/mL, about 40 to about 50 μg/ML, about 45 to about 55 μg/mL, about 50 to about 60 μg/mL, about 55 to about 65 μg/mL, about 60 to about 70 μg/mL, about 65 to about 75 μg/mL, about 70 to about 80 μg/mL, about 75 to about 85 μg/mL, about 80 to about 90 μg/mL, about 85 to about 95 μg/mL, or about 90 to about 100 μg/mL

In some aspects, the LNP intermediate, i.e., the LNP prior to the conjugation of the targeting monospecific antibodies or bispecific antibody, has a size below 100 nm. In some aspects, the LNP intermediate has a size below about 100 nm, below about 95 nm, below about 90 nm, below about 85 nm, below about 80 nm, below about 75 nm, below about 70 nm, below about 65 nm, below about 60 nm, below about 55 nm, below about 50 nm, below about 45 nm, or below about 40 nm. In some aspects, the LNP intermediate has a size of about 100 nm, about 95 nm, about 90 nm, about 85 nm, about 80 nm, about 75 nm, about 70 nm, about 65 nm, about 60 nm, about 55 nm, about 50 nm, about 45 nm, or about 40 nm. In some aspects, the LNP intermediate has a size of about 100 nm and 90 nm, about 95 nm and about 85 nm, about 90 nm and about 80 nm, about 85 nm and about 75 nm, about 80 nm and about 70 nm, about 75 nm and about 65 nm, about 70 nm and about 60 nm, about 65 nm and about 55 nm, about 60 nm and about 50 nm, about 55 nm and about 45 nm, about 50 nm and about 40 nm, about 45 nm and about 35 nm, or about 40 nm and about 30 nm.

In some aspects, the LNP intermediate has a polydispersity index (PDI) measured by Dynamic Light Scattering (DLS) of less than 0.25. In some aspects, the LNP intermediate has a PDI measured by DLS of less than about 0.25, less than about 0.24, less than about 0.23, less than about 0.22, less than about 0.21, less than about 0.20, less than about 0.19, less than about 0.18, less than about 0.17, less than about 0.16, less than about 0.15, less than about 0.14, less than about 0.13, less than about 0.12, less than about 0.11, or less than about 0.10. In some aspects, the LNP intermediate has a PDI measured by DLS of about 0.25, about 0.24, about 0.23, about 0.22, about 0.21, about 0.20, about 0.19, about 0.18, about 0.17, about 0.16, about 0.15, about 0.14, about 0.13, about 0.12, about 0.11, or about 0.10. In some aspects, the LNP intermediate has a PDI measured by DLS of about 0.10 to about 0.15, about 0.15 to about 0.20, or about 0.20 to about 0.25.

In some aspects, the LNP intermediate can encapsulate at least 90% of the payload, e.g., an mRNA payload, combined with the LNP intermediate components. In some aspects, the LNP intermediate can encapsulate e.g., about 91%, about 92%, about 93%, about 94%, about 95%, about 96%, about 97%, about 98%, about 99%, or about 100% of the payload combined with the LNP intermediate components.

In some aspects, the antibody-conjugated LNP has a size below 100 nm. In some aspects, the antibody-conjugated LNP has a size below about 100 nm, below about 95 nm, below about 90 nm, below about 85 nm, below about 80 nm, below about 75 nm, below about 70 nm, below about 65 nm, below about 60 nm, below about 55 nm, below about 50 nm, below about 45 nm, or below about 40 nm. In some aspects, the antibody-conjugated LNP has a size of about 100 nm, about 95 nm, about 90 nm, about 85 nm, about 80 nm, about 75 nm, about 70 nm, about 65 nm, about 60 nm, about 55 nm, about 50 nm, about 45 nm, or about 40 nm. In some aspects, the antibody-conjugated LNP has a size of about 100 nm and 90 nm, about 95 nm and about 85 nm, about 90 nm and about 80 nm, about 85 nm and about 75 nm, about 80 nm and about 70 nm, about 75 nm and about 65 nm, about 70 nm and about 60 nm, about 65 nm and about 55 nm, about 60 nm and about 50 nm, about 55 nm and about 45 nm, about 50 nm and about 40 nm, about 45 nm and about 35 nm, or about 40 nm and about 30 nm.

In some aspects, the antibody-conjugated LNP has a PDI by DLS of less than 0.25. In some aspects, the antibody-conjugated LNP has a PDI measured by DLS of less than about 0.25, less than about 0.24, less than about 0.23, less than about 0.22, less than about 0.21, less than about 0.20, less than about 0.19, less than about 0.18, less than about 0.17, less than about 0.16, less than about 0.15, less than about 0.14, less than about 0.13, less than about 0.12, less than about 0.11, or less than about 0.10. In some aspects, the antibody-conjugated LNP has a PDI measured by DLS of about 0.25, about 0.24, about 0.23, about 0.22, about 0.21, about 0.20, about 0.19, about 0.18, about 0.17, about 0.16, about 0.15, about 0.14, about 0.13, about 0.12, about 0.11, or about 0.10. In some aspects, the antibody-conjugated LNP has a PDI measured by DLS of about 0.10 to about 0.15, about 0.15 to about 0.20, or about 0.20 to about 0.25.

In some aspects, the antibody-conjugated LNP can encapsulate at least 90% of the mRNA payload. In some aspects, the antibody-conjugated LNP can encapsulate at least 90% of the payload, e.g., an mRNA payload, combined with the antibody-conjugated LNP. In some aspects, the antibody-conjugated LNP can encapsulate e.g., about 91%, about 92%, about 93%, about 94%, about 95%, about 96%, about 97%, about 98%, about 99%, or about 100% of the payload combined with the antibody-conjugated LNP.

In some aspects, the mRNA payload following encapsulation in the antibody-conjugated LNP is at concentration of at least 20 μg/mL In some aspects, the mRNA payload following encapsulation in the antibody-conjugated LNP is at concentration of at least about 10 μg/mL, at least about 15 μg/mL, at least about 20 μg/mL, at least about 25 μg/mL, at least about 30 μg/mL, at least about 35 μg/mL, at least about 40 μg/mL, at least about 45 μg/mL, at least about 50 μg/mL, at least about 55 μg/mL, at least about 60 μg/mL, at least about 65 μg/mL, at least about 70 μg/mL, at least about 75 μg/mL, at least about 80 μg/mL, at least about 85 μg/mL, at least about 90 μg/mL, at least about 95 μg/mL, at least about 100 μg/mL. In some aspects, the mRNA payload following encapsulation in the antibody-conjugated LNP is at concentration of about 10 μg/mL, about μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, or about 100 μg/mL. In some aspects, the mRNA payload following encapsulation in the antibody-conjugated LNP is at concentration of about 20 to about 30 μg/mL, about 25 to about 35 μg/mL, about 30 to about 40 μg/mL, about 35 to about 45 μg/mL, about 40 to about 50 μg/ML, about 45 to about 55 μg/mL, about 50 to about 60 μg/mL, about 55 to about 65 μg/mL, about 60 to about 70 μg/mL, about 65 to about 75 μg/mL, about 70 to about 80 μg/mL, about 75 to about 85 μg/mL, about 80 to about 90 μg/mL, about 85 to about 95 μg/mL, or about 90 to about 100 μg/mL

In some aspects, the antibody-conjugated LNP containing the encapsulated mRNA payload is stable at −80° C. for at least 1 week, at least 2 weeks, at least 3 weeks, at least 4 weeks, at least 5 weeks, at least 6 weeks or at least 7 weeks.

In some aspects, the mRNA payload encodes a CAR. In some aspects, the CAR is a second generation CAR. In some aspects the second generation CAR is bispecific. In some aspects, the second generation CAR comprises an antigen-binding domain that can specifically bind to human CD20 and to human CD18. In some aspects, the CAR further comprises a CD4 transmembrane region, a 4-1BB costimulatory domain, and a CD3ζ stimulatory domain. In some aspects, the mRNA encoding the CAR comprises a poly(A) having 100 nucleotides or 120 nucleotides. In some aspects, the mRNA encoding the CAR comprises a 5′ UTR selected from the group consisting of human CD3 5′ UTR, human beta-globin 5′ UTR, human alpha-globin 5′ UTR, and Adj5 major late promoter 5′ UTR. In some aspects, the mRNA encoding the CAR comprises a 5′ UTR comprising, consisting or consisting essentially of a human CD3 5′ UTR. In some aspects, the mRNA encoding the CAR comprises a 5′ UTR comprising, consisting or consisting essentially of a human beta-globin 5′ UTR. In some aspects, the mRNA encoding the CAR comprises a 5′ UTR comprising, consisting or consisting essentially of a human alpha-globin 5′ UTR. In some aspects, the mRNA encoding the CAR comprises a 5′ UTR comprising, consisting or consisting essentially of a Ad5 major late promoter 5′ UTR. In some aspects, the mRNA encoding the CAR comprises a 3′ UTR selected from the group consisting of human CD3 3′ UTR, human beta-globin 3′ UTR, and human alpha-globin 3′ UTR. In some aspects, the mRNA encoding the CAR comprises a 3′ UTR comprising, consisting or consisting essentially of a human CD3 3′ UTR. In some aspects, the mRNA encoding the CAR comprises a 3′ UTR comprising, consisting or consisting essentially of a human beta-globin 3′ UTR. In some aspects, the mRNA encoding the CAR comprises a 3′ UTR comprising, consisting or consisting essentially of a human alpha-globin 3′ UTR. In some aspects, the mRNA encoding the CAR comprises a human CD3 5′ UTR and a human CD3 3′ UTR. In some aspects, the mRNA encoding the CAR comprises a human CD3 5′ UTR, a human CD3 3′ UTR, and a 100 nucleotides long Poly(A) tail. In some aspects, the mRNA encoding the CAR comprises a human beta-globin 5′ UTR and a human beta-globin 3′ UTR. In some aspects, the mRNA encoding the CAR comprises a human beta-globin 5′ UTR, a human beta-globin 3′ UTR, and a 100 nucleotides long Poly(A) tail. In some aspects, the mRNA encoding the CAR comprises a human alpha-globin 5′ UTR and a human beta-globin 3′ UTR. In some aspects, the mRNA encoding the CAR comprises a human alpha-globin 5′ UTR, a human beta-globin 3′ UTR, and a 120 nucleotides long Poly(A) tail. In some aspects, the mRNA encoding the CAR comprises an Ad5 major late promoter 5′ UTR and a human alpha-globin 3′ UTR. In some aspects, the mRNA encoding the CAR comprises an Ad5 major late promoter 5′ UTR, a human alpha-globin 3′ UTR, and a 120 nucleotides long Poly(A) tail. In some aspects, the mRNA encoding the CAR comprises microRNA sequences to avoid expression in myeloid cells.

In some aspects, the polynucleotide encoding the CAR is codon optimized. In some aspects, the mRNA encoding a CAR comprises a HBA1 (hemoglobin subunit alpha 1) 5′ UTR, a codon optimized ORF encoding a CAR, e.g., a second generation CAR, and a 2×HBB (hemoglobin subunit beta) 3′ UTR. In some aspects, the mRNA encoding a CAR comprises a TPL (adenoviral tripartite leader) 5′ UTR, a codon optimized ORF encoding a CAR, e.g., a second generation CAR, and an HBA (hemoglobin subunit alpha) 3′ UTR. In some aspects, the mRNA encoding a CAR comprises an HBB (hemoglobin subunit beta) 5′ UTR, a codon optimized ORF encoding a CAR, e.g., a second generation CAR, and an HBB (hemoglobin subunit beta) 3′ UTR. In some aspects, upon delivery of the mRNA encapsulated into an antibody-conjugated LNP disclosed herein to human T cells, the CAR (e.g., a CD19/CD20 second generation CAR) is expressed on the surface of the T cells. In some aspects, the LNP of the present disclosure comprises (i) an LNP comprising DSPC, cholesterol, DMG-PEG2000, ad Lipid 10; (ii) an MX1500 bispecific CD3/CD28 antibody capable of targeting the LNP to T cells, wherein the antibody is conjugated to the surface of the LNP via DSPE-PEG2000-maleimide, and (iii) a payload comprising an mRNA encoding a second-generation bispecific CAR targeting CD19 and CD20 on the surface of human T cells.

The production process to generate the antibody-conjugated LNP for targeted delivery of mRNA cargo to human primary T cells or B cells disclosed herein comprises three main steps:
i. Antibody decapping to generate a free thiol from the protected thiol group from the engineered cysteine in the antibody to conjugate to the LNP.
ii. LNP preparation including formulation optimization for targeted delivery to human T cells or B cells.
iii. Conjugation/purification process to produce and achieve good yield and high-quality product.
   i. Antibody Decapping

The strategy to conjugate an antibody to an LNP intermediate to obtain and antibody-conjugated LNP of the present disclosure comprises engineering a cysteine amino acid at the C-terminus of one heavy chain, e.g., a heavy chain containing Knob mutations, for LNP conjugation. This cysteine is mostly capped by post-translational modifications such as glutathionylation or cysteinylation in protein produced in mammalian cells. Accordingly, the engineered cysteine much be decapped before the conjugation process.

In some aspects, the de-capping step is performed by using a reducing agent such as DTT or TCEP to generate the free thiol group for click chemistry with maleimide on the LNPs. To efficiently de-cap the C-terminus cysteine but also minimize the disruption of the disulfide bonds, optimizations are carried at specific temperatures molar ratios between reducing agent and antibody. In some aspects, the de-capping agent is DTT. In some aspects, de-capping is conducted at about 37° C. using DTT. In some aspects, de-capping is conducted at a molar ratio between DTT and antibody of about 5:1. In some aspects, de-capping is conducted at about 37° C. and at a molar ratio between DTT and antibody of about 5:1. In some aspects, de-capping is not conducted at a temperature below about 37° C., e.g., about 25° C. or about 4° C., using DTT. In some aspects, de-capping is not conducted at a molar ratio between DTT and antibody below about 5:1, e.g., about 1:1. In some aspects, the de-capping agent is TCEP. In some aspects, de-capping is conducted at about 37° C. using TCEP. In some aspects, de-capping is conducted below about 37° C. using TCEP. In some aspects, de-capping is conducted at about 4° C. using TCEP. In some aspects, de-capping is conducted at a molar ratio between TCEP and antibody of about 5:1, about 3:1, or about 1:1. In some aspects, de-capping is conducted at about 37° C. and at a molar ratio between TCEP and antibody of about 5:1. In some aspects, de-capping is conducted at about 37° C. and at a molar ratio between TCEP and antibody of about 3:1. In some aspects, de-capping is conducted at about 4° C. and at a molar ratio between TCEP and antibody of about 5:1. In some aspects, de-capping is conducted at about 4° C. and at a molar ratio between TCEP and antibody of about 3:1. In some aspects, de-capping is not conducted at a molar ratio between TCEP and antibody below about 5:1 or about 3:1, e.g., about 1:1. In some aspects, de-capping is conducted using a DTT/antibody molar ratio between about 3:1 and about 5:1 at about 37° C. with shaking (about 350 rpm) for about one hour. In some aspects, de-capping is conducted using a TCEP/antibody molar ratio of about 3:1 at temperature from about 4° C. to about 37° C. with shaking (about 350 rpm) for about one hour.

Lower DTT(TCEP)/Ab molar ratio and low temperature are preferred for minimizing the reduction of disulfide bonds in antibodies. It is essential to remove 99.99% of the DTT/TCEP post de-capping step due to potential interaction between DTT/TCEP and maleimide. Accordingly, in some aspects, the de-capping reaction comprises removing all the DTT or TCEP post de-capping step.

ii. LNP Formulation Optimization

One of the key attributes of a viable product for in vivo CAR-T production using antiCD3/antiCD28 for targeting to T cells is to achieve efficient gene delivery at low payload dose to avoid co-delivery of conjugated antiCD3/antiCD28, which can induce cytokine release, and is a major contributor to the observed adverse effects. In other words, it is highly desirable to achieve the highest gene delivery efficacy while using the lowest quantity of antiCD3/antiCD28. In some aspects, this can be accomplished while having an antiCD3/antiCD28 antibody to payload ratio below 1 (w/w).

LNP formulation optimization can be undertaken by varying, for example, (i) the molar % of the ionizable cationic lipid (related to potency); (ii) the molar % of the maleimide-PEG (related to antibody density and potential antibody-induced toxicity); (iii) the N/P ratio, i.e., the stoichiometry of protonatable nitrogen (N) and anionic phosphate groups (P) in a nucleic acid (necessary to achieve an adequate lipid/nucleic acid ratio); and/or (iv) the molar % of the helper lipid (related to stability and to endosomal escape). Thus, in some aspects, the manufacture of a LNP formulation of the present disclosure comprises optimizing (i) the molar % of the ionizable cationic lipid; (ii) the molar % of the maleimide-PEG; (iii) the N/P ratio; (iv) the molar % of the helper lipid, or (v) any combination thereof.

In some aspects, the LNP formulation optimization process comprises (i) ionizable cationic lipid selection, (ii) optimization of molar ratio of lipid components, and (iii) optimization of maleimide-PEG2000-DSPE percentage, thereby selecting the combination of optimized components that provide (a) minimal aggregation, (b) maximal in vitro/in vivo delivery efficacy to human primary T cells, (c) high yield (>50% recovery rate), and (d) stability under −80° C. storage conditions. In some aspects, the LNP formulation optimization process comprises evaluating a selection of ionizable cationic lipids selected from the group consisting of CKK-E12, MC3, SM-102, ACL-0315, KC2, Lipid A6, Lipid M, Lipid 10, C14-4, and any one of MDX1-MDX13.

Generally, LNP containing more ionizable cationic lipid have more effective endosomal escape, resulting in better cargo delivery to target cells. Accordingly, in some aspects, optimization is performed by maximizing the content of ionizable cationic lipid in the LNP. In some aspects, LNPs are not unconjugated LNP prepared using a lipid molar ratio for Ionizable cationic lipid:Helper lipid:Cholesterol:PEG2000-DMG of 50:10:38.5:1.5 (conventional/commercial molar ratio). In some aspects, LNPs are not unconjugated LNP prepared using a lipid molar ratio for Ionizable cationic lipid:Helper lipid:Cholesterol:PEG2000-DMG:DSPE-PEG2000-maleimide of 50:10:38.5:1.3:0.2 (conventional/commercial molar ratio).

In some aspects, LNPs are prepared using a lipid molar ratio for Ionizable cationic lipid:Helper lipid:Cholesterol:PEG2000-DMG of 35:16:46.5:2.5. In some aspects, such lipid molar ratio results in a recovery rate of at least 40% and stable conjugated products.

### ii.a Ionizable Cationic Lipid (Lipid 10) Optimization

The ionizable cationic lipid optimization phase of the LNP optimization process comprises identifying the optimal ratio of ionizable cationic lipid with respect to the rest of lipid components in the LNP. In some aspects, the ionizable cationic lipid is Lipid 10. In some aspects, Lipid 10 is at a molar ratio between 20% and 35%, whereas the Helper lipid (DSPC) is at 16%, PEG2000-DMG is at 2.3%, DSPE-PEG2000-maleimide is at 0.2%, and the molar ratio of cholesterol varies accordingly between 46.5% and 61.5%.

As used herein, the term “Lipid 10 optimized” refers to an LNP of the present disclosure in which, the molar ratio of Lipid 10 has been optimized to maximize mRNA encapsulation efficiency and mRNA recovery rate, to maximize mRNA delivery, and to minimize T cell activation.

In some aspects, the Lipid 10 optimized LNP comprises Lipid 10 at a molar ratio of at least about 20%. In some aspects, the Lipid 10 optimized LNP comprises Lipid 10 at a molar ratio of at least about 22.5%. In some aspects, the Lipid 10 optimized LNP comprises Lipid 10 at a molar ratio of at least about 25%. In some aspects, the Lipid 10 optimized LNP comprises Lipid 10 at a molar ratio of at least about 27.5%. In some aspects, the Lipid 10 optimized LNP comprises Lipid 10 at a molar ratio of at least about 30%. In some aspects, the Lipid 10 optimized LNP comprises Lipid 10 at a molar ratio of at least about 32.5%. In some aspects, the Lipid 10 optimized LNP comprises Lipid 10 at a molar ratio of at least about 35%. In some aspects, the Lipid 10 optimized LNP comprises Lipid 10 at a molar ratio of about 20%. In some aspects, the Lipid 10 optimized LNP comprises Lipid 10 at a molar ratio of about 22.5%. In some aspects, the Lipid 10 optimized LNP comprises Lipid 10 at a molar ratio of about 25%. In some aspects, the Lipid 10 optimized LNP comprises Lipid 10 at a molar ratio of about 27.5%. In some aspects, the Lipid 10 optimized LNP comprises Lipid 10 at a molar ratio of about 30%. In some aspects, the Lipid 10 optimized LNP comprises Lipid 10 at a molar ratio of about 32.5%. In some aspects, the Lipid 10 optimized LNP comprises Lipid 10 at a molar ratio of about 35%.

In some aspects, the Lipid 10 optimized LNP comprises Lipid 10 at a molar ratio between about 15% and about 17.5%. In some aspects, the Lipid 10 optimized LNP comprises Lipid 10 at a molar ratio between about 17.5% and about 20%. In some aspects, the Lipid 10 optimized LNP comprises Lipid 10 at a molar ratio between about 20% and about 22.5%. In some aspects, the Lipid 10 optimized LNP comprises Lipid 10 at a molar ratio between about 22.5% and about 25%. In some aspects, the Lipid 10 optimized LNP comprises Lipid 10 at a molar ratio between about 25% and about 27.5%. In some aspects, the Lipid 10 optimized LNP comprises Lipid 10 at a molar ratio between about 27.5% and about 30%. In some aspects, the Lipid 10 optimized LNP comprises Lipid 10 at a molar ratio between about 30% and about 32.5%. In some aspects, the Lipid 10 optimized LNP comprises Lipid 10 at a molar ratio between about 32.5% and about 35%. In some aspects, the Lipid 10 optimized LNP comprises Lipid 10 at a molar ratio between about 35% and about 37.5%. In some aspects, the Lipid 10 optimized LNP comprises Lipid 10 at a molar ratio between about 37.5% and about 40%.

In some aspects, the Lipid 10 optimized LNP comprises Lipid 10 at a molar ratio between about 15% and about 20%. In some aspects, the Lipid 10 optimized LNP comprises Lipid 10 at a molar ratio between about 20% and about 25%. In some aspects, the Lipid 10 optimized LNP comprises Lipid 10 at a molar ratio between about 25% and about 30%. In some aspects, the Lipid 10 optimized LNP comprises Lipid 10 at a molar ratio between about 30% and about 35%. In some aspects, the Lipid 10 optimized LNP comprises Lipid 10 at a molar ratio between about 35% and about 40%. In some aspects, the Lipid 10 optimized LNP comprises Lipid 10 at a molar ratio between about 10% and about 20%. In some aspects, the Lipid 10 optimized LNP comprises Lipid 10 at a molar ratio between about 20% and about 30%. In some aspects, the Lipid 10 optimized LNP comprises Lipid 10 at a molar ratio between about 30% and about 40%. In some aspects, the Lipid 10 optimized LNP comprises Lipid 10 at a molar ratio between about 40% and about 50%.

In some aspects, the Lipid 10 optimized LNP comprises Lipid 10 at a 20% molar ratio and the antiCD3/antiCD28 antibody covalently attached to the surface of the LNP is an MSTAR, e.g., MX1500. In some aspects, the Lipid 10 optimized LNP comprises Lipid 10 at a 22.5% molar ratio and the antiCD3/antiCD28 antibody covalently attached to the surface of the LNP is MX1500. In some aspects, the Lipid 10 optimized LNP comprises Lipid 10 at a 25% molar ratio and the antiCD3/antiCD28 antibody covalently attached to the surface of the LNP is MX1500. In some aspects, the Lipid 10 optimized LNP comprises Lipid 10 at a 27.5% molar ratio and the antiCD3/antiCD28 antibody covalently attached to the surface of the LNP is MX1500. In some aspects, the Lipid 10 optimized LNP comprises Lipid 10 at a 30% molar ratio and the antiCD3/antiCD28 antibody covalently attached to the surface of the LNP is MX1500. In some aspects, the Lipid 10 optimized LNP comprises Lipid 10 at a 32.5% molar ratio and the antiCD3/antiCD28 antibody covalently attached to the surface of the LNP is MX1500. In some aspects, the Lipid 10 optimized LNP comprises Lipid 10 at a 35% molar ratio and the antiCD3/antiCD28 antibody covalently attached to the surface of the LNP is MX1500. In some aspects, the Lipid 10 optimized LNP comprises Lipid 10 at a 30% molar ratio and the antiCD3/antiCD28 antibody covalently attached to the surface of the LNP is MX1500. In some aspects, the Lipid 10 optimized LNP comprises Lipid 10 at a 32.5% molar ratio and the antiCD3/antiCD28 antibody covalently attached to the surface of the LNP is MX1500. In some aspects, the Lipid 10 optimized LNP comprises Lipid 10 at a 35% molar ratio and the antiCD3/antiCD28 antibody covalently attached to the surface of the LNP is MX1500.

In some aspects, the payload comprises an mRNA at a concentration between about 0.001 μg/mL and about 1 μg/mL. In some aspects, the mRNA payload is at a concentration selected from the group consisting of about 0.002 μg/mL, about 0.008 μg/mL, about 0.03 μg/mL, about 0.125 μg/mL, or about 0.5 μg/mL In some aspects, the mRNA payload is at a concentration selected from the group consisting of about 0.001 μg/mL, about 0.002 μg/mL, about 0.005 μg/mL, about 0.008 μg/mL, about 0.01 μg/mL, about 0.02 μg/mL, about 0.05 μg/mL, about 0.1 μg/mL, about 0.125 μg/mL, about 0.150 μg/mL, about 0.175 μg/mL, about 0.200 μg/mL, about 0.250 μg/mL, about 0.300 μg/mL, about 0.350 μg/mL, about 0.400 μg/mL, about 0.450 μg/mL, about 0.500 μg/mL, about 0.600 μg/mL, about 0.700 μg/mL, about 0.800 μg/mL, about 0.900 μg/mL, and about 1 μg/mL. In some aspects, the mRNA payload is at a concentration selected between about 0.001 μg/mL and about 0.002 μg/mL, about 0.002 μg/mL and about 0.005 μg/mL, about 0.005 μg/mL and about 0.008 μg/mL, about 0.008 μg/mL and about 0.01 μg/mL, about 0.01 μg/mL and about 0.02 μg/mL, about 0.02 μg/mL and about 0.05 μg/mL, about 0.05 μg/mL and about 0.1 μg/mL, about 0.1 μg/mL and about 0.125 μg/mL, about 0.125 μg/mL and about 0.150 μg/mL, about 0.150 μg/mL and about 0.175 μg/mL, about 0.175 μg/mL and about 0.200 μg/mL, about 0.200 μg/mL and about 0.250 μg/mL, about 0.250 μg/mL and about 0.300 μg/mL, about 0.300 μg/mL and about 0.350 μg/mL, about 0.350 μg/mL and about 0.400 μg/mL, about 0.400 μg/mL and about 0.450 μg/mL, about 0.450 μg/mL and about 0.500 μg/mL, about 0.500 μg/mL and about 0.600 μg/mL, about 0.600 μg/mL and about 0.700 μg/mL, about 0.700 μg/mL and about 0.800 μg/mL, about 0.800 μg/mL and about 0.900 μg/mL, about 0.900 μg/mL and about 1 μg/mL.

Biophysical properties of exemplary LNP particles of the present disclosure are summarized in the table below.

TABLE 11
Biophysical properties of LNP. LNPs without a conjugated antibody (labeled “None”) are
“LNP intermediates.” LNPs with MX1500 conjugated to their surface are “antibody-conjugated LNPs.”

| | | Conjugated | | | | | |
|---|---|---|---|---|---|---|---|
| | Lipid 10 | bispecific | | | | mRNA | mRNA |
| | molar | antiCD3/ | | Polydispersity | | Encapsulation | Recovery |
| | ratio | antiCD28 | Size | Index | Charge | Efficiency | Rate |
| LNP | (%) | antibody | (nm) | (PI) | (mV) | (%) | (%) |
| Lipid10_27.5_0.20 | 27.5 | None | 78 | 0.03 | −3.28 | 99 | 65.9 |
| Lipid10_27.5_0.20_MX1500 | 27.5 | MX1500 | 101 | 0.15 | −0.93 | 97 | 60.2 |
| Lipid10_25_0.20 | 25 | None | 77 | 0.08 | −2.52 | 99 | 68.4 |
| Lipid10_25_0.20_MX1500 | 25 | MX1500 | 104 | 0.12 | −1.67 | 97 | 61.1 |
| Lipid10_22.5_0.20 | 22.5 | None | 77 | 0.06 | −1.76 | 99 | 62.4 |
| Lipid10_22.5_0.20_MX1500 | 22.5 | MX1500 | 101 | 0.10 | −0.76 | 97 | 69.7 |
| Lipid10_20_0.20 | 20 | None | 78 | 0.08 | −3.05 | 99 | 59.6 |
| Lipid10_20_0.20_MX1500 | 20 | MX1500 | 100 | 0.11 | −1.11 | 97 | 73.9 |
| Lipid 10_27.5 | 27.5 | None | 72 | 0.07 | −1.84 | 99 | 75 |
| Lipid 10_27.5_MX1500 | 27.5 | MX1500 | 92 | 0.12 | −1.01 | 98 | 74 |
| Lipid 10_30 | 30 | None | 72 | 0.02 | −3.53 | 99 | 81 |
| Lipid 10_30_MX1500 | 30 | MX1500 | 89 | 0.08 | −1.25 | 98 | 62 |
| Lipid 10_32.5 | 32.5 | None | 71 | 0.05 | −2.69 | 99 | 76 |
| Lipid 10_32.5_MX1500 | 32.5 | MX1500 | 88 | 0.10 | −1.6 | 98 | 69 |
| Lipid 10_35 | 35 | None | 71 | 0.04 | −0.48 | 99 | 96 |
| Lipid 10_35_MX1500 | 35 | MX1500 | 85 | 0.09 | 1.24 | 99 | 60 |
| Lipid 10_37.5 | 37.5 | None | 77 | 0.02 | −1.67 | 99 | 79 |
| Lipid 10_37.5_MX1500 | 37.5 | MX1500 | 87 | 0.03 | −1.83 | 96 | 43 |

In some aspects, the Lipid 10 optimized LNP is an LNP intermediate having a size between about 71 nm and about 78 nm. In some aspects, the Lipid 10 optimized LNP is an LNP intermediate having a size between about 65 nm and about 85 nm. In some aspects, the Lipid 10 optimized LNP is an LNP intermediate having a size of about 65 nm, about 70 nm, about 75 nm, about 80 nm, or about 85 nm. In some aspects, the Lipid 10 optimized LNP is an LNP intermediate having a size between about 65 nm and about 70 nm, between about 70 nm and about 75 nm, between about 75 nm and about 80 nm, between about 80 nm and about 85 nm, between about 65 nm and about 75 nm, or between about 75 nm and about 85 nm.

In some aspects, the Lipid 10 optimized LNP is an antibody-conjugated LNP (e.g., a LNP intermediate conjugated to bispecific antiCD3/antiCD28 antibody, e.g., an MSTAR such as MX1500) having a size between about 85 nm and about 104 nm. In some aspects, the Lipid 10 optimized LNP is an antibody-conjugated LNP having a size between about 80 nm and about 110 nm. In some aspects, the Lipid 10 optimized LNP is an antibody-conjugated LNP having a size of about 80 nm, about 85 nm, about 90 nm, about 95 nm, about 100 nm, about 105 nm, or about 110 nm. In some aspects, the Lipid 10 optimized LNP is an antibody-conjugated LNP having a size between about 80 nm and about 85 nm, between about 85 nm and about 90 nm, between about 90 nm and about 95, between about 95 nm and about 100 nm, between about 100 nm and about 105 nm, between about 105 nm and about 110 nm, between about 80 nm and about 90 nm, between about 90 nm and about 100 nm, between about 100 nm and about 110, between about 80 nm and about 100 nm, between about 90 nm and about 110 nm, between about 85 nm and about 105 nm.

In some aspects, the Lipid 10 optimized LNP is an LNP intermediate having a polydispersity index between about 0.02 and about 0.08. In some aspects, the Lipid 10 optimized LNP is an LNP intermediate having a polydispersity index of about 0.02, about 0.03, about 0.04, about 0.05, about 0.06, about 0.07, or about 0.08. In some aspects, the Lipid 10 optimized LNP is an antibody-conjugated LNP (e.g., a LNP intermediate conjugated to bispecific antiCD3/antiCD28 antibody, e.g., an MSTAR such as MX1500) having a polydispersity index between about 0.03 and about 0.15. In some aspects, the Lipid 10 optimized LNP is an antibody-conjugated LNP having a polydispersity index of about 0.03, about 0.04, about 0.05, about 0.06, about 0.07, about 0.08, about 0.09, about 0.10, about 0.11, about 0.12, about 0.13, about 0.14, or about 0.15.

In some aspects, the Lipid 10 optimized LNP is an LNP intermediate having a charge between about −3.53 mV and about −0.48 mV. In some aspects, the Lipid 10 optimized LNP is an LNP intermediate having a charge of about −3.6 mV, about −3.4 mV, about −3.2 mV, about −3 mV, about −2.75 mM, about −2.5 mV, about −2.25 mV, about −2 mV, about −1.75 mV, about −1.5 mV, about −1.25 mV, about −1 mV, about −0.75 mV, or about −0.50 mV. In some aspects, the Lipid 10 optimized LNP is an antibody-conjugated LNP (e.g., a LNP intermediate conjugated to bispecific antiCD3/antiCD28 antibody, e.g., an MSTAR such as MX1500) having a charge between about −1.83 mV and about 1.24 mV. In some aspects, the Lipid 10 optimized LNP is an antibody-conjugated LNP having a charge of about −2 mV, about −1.75 mV, about −1.5 mV, about −1.25 mV, about −1 mV, about −0.75 mV, about −0.5 mV, about −0.25 mV, about 0 mV, about 0.25 mV, about 0.5 mV, about 0.75 mV, about 1 mV, or about 1.25 mV.

In some aspects, the Lipid 10 optimized LNP is an LNP intermediate having an mRNA encapsulation efficiency about 99%. In some aspects, the Lipid 10 optimized LNP is an antibody-conjugated LNP (e.g., a LNP intermediate conjugated to bispecific antiCD3/antiCD28 antibody, e.g., an MSTAR such as MX1500) having an mRNA encapsulation efficiency between about 96% and about 99%. In some aspects, the Lipid 10 optimized LNP is an antibody-conjugated LNP having a mRNA encapsulation efficiency of about 95%, about 96%, about 97%, about 98%, or about 99%.

In some aspects, the Lipid 10 optimized LNP is an LNP intermediate having an mRNA recovery rate between about 60% and about 96%. In some aspects, the Lipid 10 optimized LNP is an LNP intermediate having a mRNA recovery rate of about 55%, about 60%, about 65%, about 70%, about 75%, about 80%, about 85%, about 90%, about 91%, about 92%, about 93%, about 94%, about 95%, about 96%, about 97%, about 98%, or about 99%

In some aspects, the Lipid 10 optimized LNP is an antibody-conjugated LNP (e.g., a LNP intermediate conjugated to bispecific antiCD3/antiCD28 antibody, e.g., an MSTAR such as MX1500) having an mRNA recovery rate between about 43% and about 74%. In some aspects, the Lipid 10 optimized LNP is an antibody-conjugated LNP having a mRNA recovery rate of about 40%, about 45%, about 50%, about 55%, about 60%, about 65%, about 70%, about 75%, or about 80%.

In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 20% Lipid, and the mRNA delivery rate to peripheral human T cells is between about 15% and about 30%. In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 20% Lipid, and the mRNA delivery rate to peripheral human T cells is about 15%, about 20%, about 25%, or about 30%. In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 20% Lipid, and the mRNA delivery rate to peripheral human T cells is between about 15% and about 20%, between about 20% and about 25%, or between about 25% and about 30%.

In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 27.5% Lipid, and the mRNA delivery rate to peripheral human T cells is between about 30% and about 60%. In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 27.5% Lipid, and the mRNA delivery rate to peripheral human T cells is about 30%, about 35%, about 40%, about 45%, about 50%, about 55%, or about 60%. In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 27.5% Lipid, and the mRNA delivery rate to peripheral human T cells is between about 30% and about 35%, between about 35% and about 40%, between about 40% and about 45%, between about 45% and about 50%, between about 50% and about 55%, or between about 55% and about 60%.

In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 30% Lipid, and the mRNA delivery rate to peripheral human T cells is between about 30% and about 40%. In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 30% Lipid, and the mRNA delivery rate to peripheral human T cells is about 30%, about 35%, or about 40%. In some aspects, the Lipid 10 optimized LNP is an antibody-conjugated LNP, e.g., a LNP intermediate conjugated to bispecific antiCD3/antiCD28 antibody, e.g., an MSTAR such as MX1500, wherein the LNP comprises about 30% Lipid, and the mRNA delivery rate to peripheral human T cells is between about 30% and about 35%, or between about 35% and about 40%.

In LNP formulations at a fixed molar % of DSPC (about 16%), DSPE-PEG2000-maleimide (about 0.2%) and DMG-PEG2000 (about 2.3%), molar % reduction of Lipid 10 (from 35% to 20%) and corresponding molar % increase of cholesterol (from 46.5-61.5%) correlate with increased in vitro delivery efficacy to primary human T cells. Furthermore, molar % reduction of Lipid 10 (from 35 to 20%) and molar % increase of cholesterol (from 46.5-61.5%) results in improved yield of the antibody conjugated LNPs (improved recovery rate). Accordingly, in some aspects, intermediate LNPs of the present disclosure comprise 16% DSPC, 0.2% DSPE-PEG2000-maleimide, 2.3% DMG-PEG2000 (about 2.3%), 20% Lipid 10 and 61.5% cholesterol. In some aspects, antibody-conjugated LNPs of the present disclosure comprise 16% DSPC, 0.2% DSPE-PEG2000-antibody (wherein the antibody is a bispecific antiCD3/antiCD28 antibody, e.g., an MSTAR such as MX1500), 2.3% DMG-PEG2000 (about 2.3%), 20% Lipid 10 and 61.5% cholesterol.

### ii.B Maleimide-PEG (DSPE-PEG2000-Maleimide) Optimization

In some aspects, LNP compositions of the presents disclosure have been optimized for antibody conjugation, e.g., the molar ratio of Lipid-PEG-maleimide (e.g., DSPE-PEG2000-maleimide) has been modified to identify the optimal amount of lipid available for conjugation to the T cell targeting antibody (for example, a bispecific antiCD3/antiCD28 antibody, e.g., an MSTAR such as MX1500) or antibodies (e.g., a monospecific antiCD3 and a monospecific antiCD28, which can be in MSTAR format).

As used herein the term “maleimide-PEG optimized” refers to an LNP of the present disclosure in which the molar rates (%) of DSPE-PEG2000-maleimide and DMG-PEG2000 have been optimize to maximize mRNA encapsulation efficiency and mRNA recovery rate, to maximize mRNA delivery, and to minimize T cell activation.

In some aspects, the present disclosure provides a maleimide-PEG optimized LNP comprising a molar ratio (%) of about 54%, about 16% helper lipid (DSPC) and about 27.5% lipid, wherein the molar ratio of DSPE-PEG2000-maleimide is from about 0.05% to about 0.2%, and the molar ratio of DMG-PEG2000, wherein the total molar ratio of DSPE-PEG2000-maleimide plus DMG-PEG2000 is about 2.5%. In some aspects, the maleimide-PEG optimized LNP has a molar ratio of DSPE-PEG2000-maleimide of about 0.05%. In some aspects, the maleimide-PEG optimized LNP has a molar ratio of DSPE-PEG2000-maleimide of about 0.10%. In some aspects, the maleimide-PEG optimized LNP has a molar ratio of DSPE-PEG2000-maleimide of about 0.15%. In some aspects, the maleimide-PEG optimized LNP has a molar ratio of D SPE-PEG2000-maleimide of about 0.20%. In some aspects, the maleimide-PEG optimized LNP, e.g., has a molar ratio of DSPE-PEG2000-maleimide of about 0.05% and it is antibody-conjugated, e.g., to a bispecific antiCD3/antiCD28 antibody, e.g., an MSTAR such as MX1500. In some aspects, the maleimide-PEG optimized LNP has a molar ratio of DSPE-PEG2000-maleimide of about 0.10% and it is antibody-conjugated, e.g., to a bispecific antiCD3/antiCD28 antibody, e.g., an MSTAR such as MX1500. In some aspects, the maleimide-PEG optimized LNP has a molar ratio of DSPE-PEG2000-maleimide of about 0.15% and it is antibody-conjugated, e.g., to a bispecific antiCD3/antiCD28 antibody, e.g., an MSTAR such as MX1500. In some aspects, the maleimide-PEG optimized LNP has a molar ratio of DSPE-PEG2000-maleimide of about 0.20% and it is antibody-conjugated, e.g., to a bispecific antiCD3/antiCD28 antibody, e.g., an MSTAR such as MX1500.

In some aspects, the maleimide-PEG optimized LNP has an mRNA payload of about 0.008, about 0.03, or about 0.125 μg/mL. In some aspects, the maleimide-PEG optimized LNP has an mRNA delivery efficacy of at least about 40%, at least about 45%, at least about 50%, at least about 55%, at least about 60%, at least 65%, at least about 70%, at least about 75%, at least about 80%, at least about 85%, at least about 90%, or at least about 95%. In some aspects, the maleimide-PEG optimized LNP has an mRNA delivery efficacy of about 40% to about 95%. In some aspects, the LNP has an mRNA delivery efficacy of about 40%, about 45%, about 50%, about 55%, about 60%, about 65%, about 70%, about 75%, about 80%, about 85%, about 90%, or about 95%. In some aspects, the maleimide-PEG optimized LNP has an mRNA delivery efficacy between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, or between about 95% and about 100%.

In some aspects, the maleimide-PEG optimized LNP has a T cell activation efficacy of at least about 15%, at least about 20%, at least about 25%, at least about 30%, at least about 35%, at least about 40%, at least about 45%, at least about 50%, at least about 55%, or at least about 60%. In some aspects, the maleimide-PEG optimized LNP has an mRNA delivery efficacy of about 15% to about 60%. In some aspects, the maleimide-PEG optimized LNP has a T cell activation efficacy of about 15%, about 20%, about 25%, about 30%, about 35%, about 40%, about 45%, about 50%, about 55%, or about 60%. %. In some aspects, the maleimide-PEG optimized LNP has an mRNA delivery efficacy between about 15% and about 20%, between about 20% and about 25%, between about 25% and about 30%, between about 30% and about 35%, between about 35% and about 40%, between about 40% and about 45%, between about 45% and about 50%, between about 50% and about 55%, between about 55% and about 60%, between about 20% and about 30%, between about 30% and about 40%, between about 40% and about 50%, between about 50% and about 60%, or, between about 60% and about 70%.

Physicochemical characteristics of maleimide-PEG (DSPE-PEG2000-Maleimide) molar ratio optimized LNP of the present disclosure are summarized the table below.

TABLE 12
Physicochemical characteristics of maleimide-PEG optimized LNP.

| | | | | mRNA | |
|---|---|---|---|---|---|
| | | | | Encap- | mRNA |
| | | Polydis- | | sulation | Re- |
| | | persity | | Effi- | covery |
| | Size | Index | Charge | ciency | Rate |
| LNP | (nm) | (PI) | (mV) | (%) | (%) |
| Lipid10_27.5_0% Mal | 78 | 0.04 | −2.5 | 99 | 59.5 |
| Lipid10_27.5_0% | 81 | 0.09 | −3.71 | 97 | 56.3 |
| Mal_MX1500 | | | | | |
| Lipid10_27.5_0.05% Mal | 83 | 0.07 | −2.51 | 99 | 52.1 |
| Lipid10_27.5_0.05% | 89 | 0.10 | −1.65 | 99 | 73.6 |
| Mal_MX1500 | | | | | |
| Lipid10_27.5_0.10% Mal | 82 | 0.05 | −1.61 | 99 | 66.6 |
| Lipid10_27.5_0.10% | 92 | 0.07 | −0.26 | 98 | 69.8 |
| Mal_MX1500 | | | | | |
| Lipid10_27.5_0.15% Mal | 80 | 0.04 | −2.27 | 99 | 56.3 |
| Lipid10_27.5_0.15% | 98 | 0.10 | −2.12 | 97 | 69.9 |
| Mal_MX1500 | | | | | |
| Lipid10_27.5_0.20% Mal | 78 | 0.03 | −3.28 | 99 | 65.9 |
| Lipid10_27.5_0.20% | 101 | 0.15 | −0.93 | 97 | 60.2 |
| Mal_MX1500 | | | | | |

In some aspects, the maleimide-PEG optimized LNP is an LNP intermediate having a size between about 78 nm and about 83 nm. In some aspects, the maleimide-PEG optimized LNP is an LNP intermediate having a size between about 70 nm and about 90 nm. In some aspects, the maleimide-PEG optimized LNP is an LNP intermediate having a size of about 70 nm, about 75 nm, about 80 nm, about 85 nm, or about 90 nm. In some aspects, the maleimide-PEG optimized LNP is an LNP intermediate having a size between about 70 nm and about 75 nm, between about 75 nm and about 80 nm, between about 80 nm and about 85 nm, between about 85 nm and about 90 nm, between about 70 nm and about 80 nm, or between about 80 nm and about 90 nm.

In some aspects, the maleimide-PEG optimized LNP is an antibody-conjugated LNP (e.g., a LNP intermediate conjugated to a bispecific antiCD3/antiCD28 antibody, e.g., an MSTAR such as MX1500) having a size between about 89 nm and about 101 nm. In some aspects, the maleimide-PEG optimized LNP is an antibody-conjugated LNP having a size between about 85 nm and about 105 nm. In some aspects, the maleimide-PEG optimized LNP is an antibody-conjugated LNP having a size of about 85 nm, about 90 nm, about 95 nm, about 95 nm, about 100 nm, or about 105 nm. In some aspects, the maleimide-PEG optimized LNP is an antibody-conjugated LNP having a size between about 85 nm and about 90 nm, between about 90 nm and about 95, between about 95 nm and about 100 nm, between about 100 nm and about 105 nm, between about 85 nm and about 95 nm, between about 90 nm and about 100 nm, between about 95 nm and about 105, or between about 85 nm and about 105 nm. In some aspects, the maleimide-PEG optimized LNP is an LNP intermediate having a polydispersity index between about 0.03 and about 0.07. In some aspects, the maleimide-PEG optimized LNP is an LNP intermediate having a polydispersity index of about 0.02, about 0.03, about 0.04, about 0.05, about 0.06, about 0.07, or about 0.08. In some aspects, the maleimide-PEG optimized LNP is an antibody-conjugated LNP (e.g., a LNP intermediate conjugated to a bispecific antiCD3/antiCD28 antibody, e.g., an MSTAR such as MX1500) having a polydispersity index between about 0.07 and about 0.15. In some aspects, the maleimide-PEG optimized LNP is an antibody-conjugated LNP having a polydispersity index of about 0.07, about 0.08, about 0.09, about 0.10, about 0.11, about 0.12, about 0.13, about 0.14, or about 0.15.

In some aspects, the maleimide-PEG optimized LNP is an LNP intermediate having a charge between about −3.28 mV and about −1.61 mV. In some aspects, the maleimide-PEG optimized LNP is an LNP intermediate having a charge of about −3.3 mV, about −3.2 mV, about −3.1 mV, about −3 mV, about −2.75 mM, about −2.5 mV, about −2.25 mV, about −2 mV, about −1.9 mV, about −1.8 mV, about −1.7 mV, about −1.6 mV, or about −1.5 mV. In some aspects, the maleimide-PEG optimized LNP is an antibody-conjugated LNP (e.g., a LNP intermediate conjugated to a bispecific antiCD3/antiCD28 antibody, e.g., an MSTAR such as MX1500) having a charge between about −2.12 mV and about −1.65 mV. In some aspects, the maleimide-PEG optimized LNP is an antibody-conjugated LNP having a charge of about −2.2 mV, about −2.1 mV, about −2.0 mV, about −1.9 mV, about −1.8 mV, about −1.7 mV, about −1.6 mV, or about −1.5 mV.

In some aspects, the maleimide-PEG optimized LNP is an LNP intermediate having an mRNA encapsulation efficiency about 99%. In some aspects, the maleimide-PEG optimized LNP is an antibody-conjugated LNP (e.g., a LNP intermediate conjugated to a bispecific antiCD3/antiCD28 antibody, e.g., an MSTAR such as MX1500) having an mRNA encapsulation efficiency between about 97% and about 99%. In some aspects, the maleimide-PEG optimized LNP is an antibody-conjugated LNP having an mRNA encapsulation efficiency of about 96%, about 97%, about 98%, or about 99%. In some aspects, the maleimide-PEG optimized LNP is an LNP intermediate having an mRNA recovery rate between about 50% and about 70%. In some aspects, the maleimide-PEG optimized LNP is an LNP intermediate having a mRNA recovery rate of about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%. In some aspects, the maleimide-PEG optimized LNP is an antibody-conjugated LNP (e.g., a LNP intermediate conjugated to a bispecific antiCD3/antiCD28 antibody, e.g., an MSTAR such as MX1500) having an mRNA recovery rate between about 60% and about 74%. In some aspects, the maleimide-PEG optimized LNP is an antibody-conjugated LNP having an mRNA recovery rate of about 55%, about 60%, about 65%, about 70%, or about 75%.

Reduction of maleimide-PEG (e.g., DSPE-PEG2000-Maleimide), which directly relates to the number or ratio of antibody molecules per LNP, correlates with (i) increased human primary T cell delivery efficiency and (ii) decrease T cell activation, which is a source of potential adverse events. In some aspects, the optimal maleimide-PEG (e.g., DSPE-PEG2000-Maleimide) molar ratio for in vitro testing or administration is about 0.1% or about 0.2%. In some aspects, the optimal maleimide-PEG (e.g., DSPE-PEG2000-Maleimide) molar ratio for in vivo administration is about 0.05%.

### ii.C Helper Lipid (DSPC) Optimization

In some aspects, the molar ratio of helper lipid (e.g., DSPC) in the LNP compositions of the present disclosure has been optimized. As used herein the term “helper lipid optimized” refers to an LNP of the present disclosure in which the molar rate (%) of helper lipid (e.g., DSPC) has been optimized to maximize mRNA encapsulation efficiency and mRNA recovery rate, to maximize mRNA delivery, and to minimize T cell activation. In some aspects, a helper lipid optimized LNP of the present disclosure comprises about 56.5% or 46.5% cholesterol, about 0.1% or 0.2% DSPE-PEG-maleimide, about 25% Lipid 10, and about 2.4 or about 2.3 DMG-PEG2000, and the lipid help is about 16% or about 26% DSPC. In some aspects, the helper lipid optimized LNP has a composition disclosed in the table below. In some aspects, the helper lipid optimized LNP of the table below comprises a bispecific antiCD3/antiCD28 antibody, e.g., an MSTAR such as MX1500, covalently attached to its surface via a maleimide group. In some aspects, the helper lipid optimized LNP of the table below comprises (encapsulates) an mRNA. In some aspects, the N/P ratio is about 9. In some aspects, the helper lipid optimized LNP of the table below has the ionizable cationic lipid to mRNA weight ratio of about 10.

Exemplary LNPs having different ratios of helper lipid are exemplified in TABLE 13. In some aspects, the LNP has a ratio of ionizable cationic lipid (I): helper lipid (H): cholesterol (C): DMG-PEG2000 (P1): DSPE-PEG(2000)-Maleimide (P2) of 25: 16:56.5:2.4:0.1. In some aspects, the LNP has a ratio of ionizable cationic lipid (I): helper lipid (H): cholesterol (C): DMG-PEG2000 (P1): DSPE-PEG(2000)-Maleimide (P2) of 25:26:46.5:2.4:0.1. In some aspects, the LNP has a ratio of ionizable cationic lipid (I): helper lipid (H): cholesterol (C): DMG-PEG2000 (P1): DSPE-PEG(2000)-Maleimide (P2) of 25:16:56.5:2.45:0.05. In some aspects, the LNP has a ratio of ionizable cationic lipid (I): helper lipid (H): cholesterol (C): DMG-PEG2000 (P1): DSPE-PEG(2000)-Maleimide (P2) of 25:26:46.5:2.45:0.05. In some aspects, the LNP has a ratio of ionizable cationic lipid (I): helper lipid (H): cholesterol (C): PEG lipids of 25:16:56.5:2.5. In some aspects, the LNP has a ratio of ionizable cationic lipid (I): helper lipid (H): cholesterol (C): PEG lipids of 25:26:46.5:2.5.

TABLE 13
Composition of helper lipid optimized LNPs.

| Ionizable | | | | | |
|---|---|---|---|---|---|
| cationic lipids | | | | | |
| Ionizable | Helper | | PEG Lipids | | |
| cationic lipid | Lipid | Cholesterol | DMG PEG 2000 | DSPE-PEG2000-Maleimide | Lipids Molar Ratio |
| (I) | (H) | (C) | (P1) | (P2) | (I:H:C:P1:P2) |
| Lipid 10 | DSPC | Cholesterol | DMG PEG 2000 | DSPE-PEG2000-Maleimide | 25:16:56.5:2.4:0.1 |
| Lipid 10 | DSPC | Cholesterol | DMG PEG 2000 | DSPE-PEG2000-Maleimide | 25:26:46.5:2.4:0.1 |
| Lipid 10 | DSPC | Cholesterol | DMG PEG 2000 | DSPE-PEG2000-Maleimide | 25:16:56.5:2.45:0.05 |
| Lipid 10 | DSPC | Cholesterol | DMG PEG 2000 | DSPE-PEG2000-Maleimide | 25:26:46.5:2.45:0.05 |

Physicochemical characteristics of helper lipid optimized LNPs of the present disclosure are summarized the table below.

TABLE 14
Physicochemical characteristics of helper lipid optimized LNP.

| | | | | | mRNA | mRNA |
|---|---|---|---|---|---|---|
| | | Polydispersity | | Particle | Encapsulation | Recovery |
| | Size | Index | Charge | Concentration | Efficiency | Rate |
| LNP | (nm) | (PI) | (mV) | (Particles/ml) | (%) | (%) |
| Lipid10_25_16_0.1 | 69 | 0.09 | −1.31 | 1.39E+13 | 99 | 81 |
| Lipid10_25_16_0.1_MX1500 | 120 | 0.11 | −2.82 | 2.41E+12 | 98 | 87 |
| Lipid10_25_26_0.1 | 76 | 0.08 | −1 | 2.78E+13 | 99 | 86 |
| Lipid10_25_26_0.1 MX1500 | 128 | 0.08 | −2.89 | 2.21E+12 | 98 | 67 |
| Lipid10_25_16_0.05 | 72 | 0.08 | −2.14 | 3.85E+13 | 99 | 85 |
| Lipid10_25_16_0.05 MX1500 | 104 | 0.06 | −4.59 | 6.70E+12 | 98 | 70 |
| Lipid10_25_26_0.05 | 73 | 0.08 | −4.13 | 2.27E+13 | 99 | 79 |
| Lipid10_25_26_0.05_MX1500 | 106 | 0.08 | −2.9 | 2.82E+12 | 98 | 89 |

In some aspects, the helper lipid optimized LNP is an LNP intermediate having a size between about 69 nm and about 76 nm. In some aspects, the helper lipid optimized LNP is an antibody-conjugated LNP (e.g., a LNP intermediate conjugated to a bispecific antiCD3/antiCD28 antibody, e.g., an MSTAR such as MX1500) having a size between about 104 nm and about 128 nm.

In some aspects, the helper lipid optimized LNP is an LNP intermediate having a polydispersity index between about 0.08 and about 0.09. In some aspects, the helper lipid optimized LNP is an antibody-conjugated LNP (e.g., a LNP intermediate conjugated to a bispecific antiCD3/antiCD28 antibody, e.g., an MSTAR such as MX1500) having a polydispersity index between about 0.06 and about 0.11. In some aspects, the helper lipid optimized LNP is an LNP intermediate having a charge between about −4.13 mV and about −1 mV. In some aspects, the helper lipid optimized LNP is an antibody-conjugated LNP (e.g., a LNP intermediate conjugated to a bispecific antiCD3/antiCD28 antibody, e.g., an MSTAR such as MX1500) having a charge between about −4.59 mV and about −2.82 mV. In some aspects, the helper lipid optimized LNP is an LNP intermediate having an mRNA encapsulation efficiency about 99%. In some aspects, the helper lipid optimized LNP is an antibody-conjugated LNP (e.g., a LNP intermediate conjugated to a bispecific antiCD3/antiCD28 antibody, e.g., an MSTAR such as MX1500) having an mRNA encapsulation efficiency between about 98% and about 99%. In some aspects, the helper lipid optimized LNP is an LNP intermediate having an mRNA recovery rate between about 79% and about 86%. In some aspects, the helper lipid optimized LNP is an antibody-conjugated LNP (e.g., a LNP intermediate conjugated to bispecific antiCD3/antiCD28 antibody, e.g., an MSTAR such as MX1500) having an mRNA recovery rate between about 67% and about 89%.

Increasing the molar % of DSPC in Lipid 10 LNP formulations results in reduced in vitro mRNA delivery efficiency to human primary T cells. Increasing the molar % of DSPC in Lipid 10 LNP formulations reduces the T cell activation potential of the conjugated Lipid 10 LNPs in vitro.

### ii.D N/P Ratio Optimization

In some aspects, the N/P ratio of LNP compositions of the present disclosure has been optimized. As used herein, the term “N/P ratio” refers to the ratio of elemental nitrogen and phosphate, i.e., the ratio of positively charged amino groups in the LNP lipids to negatively charged phosphate groups in a LNP of the present disclosure. This ratio describes the charge interaction between the cationic charge of the amino (N+) group in the ionizable amino-lipid to the anionic charge of the phosphate (PO⁴⁻) groups in the backbone of nucleic acids and is the basis of the complexation of the nucleic acid payload (e.g., an mRNA) with the ionizable amino-lipid.

As used herein the term “N/P ratio optimized” refers to an LNP of the present disclosure in which the N/P ratio has been optimized to maximize mRNA encapsulation efficiency and mRNA recovery rate, to maximize mRNA delivery, and to minimize T cell activation. Different N/P ratios in LNPs of the present disclosure are summarized in TABLE 15.

In some aspects, the LNP has a ratio of ionizable cationic lipid (I): helper lipid (H): cholesterol (C): DMG-PEG2000 (P1): DSPE-PEG(2000)-Maleimide (P2) of 25:16:56.5:2.4:0.1 and the N/P is 4.5. In some aspects, the LNP has a ratio of ionizable cationic lipid (I): helper lipid (H): cholesterol (C): PEG lipids of 25:16:56.5:2.5 and the N/P is 4.5. In some aspects, the LNP has a ratio of ionizable cationic lipid (I): helper lipid (H): cholesterol (C): DMG-PEG2000 (P1): DSPE-PEG(2000)-Maleimide (P2) of 25:16:56.5:2.4:0.1 and the N/P is 7. In some aspects, the LNP has a ratio of ionizable cationic lipid (I): helper lipid (H): cholesterol (C): PEG lipids of 25:16:56.5:2.5 and the N/P is 7. In some aspects, the LNP has a ratio of ionizable cationic lipid (I): helper lipid (H): cholesterol (C): DMG-PEG2000 (P1): DSPE-PEG2000-Maleimide (P2) of 25:16:56.5:2.4:0.1 and the N/P is 9. In some aspects, the LNP has a ratio of ionizable cationic lipid (I): helper lipid (H): cholesterol (C): PEG lipids of 25:16:56.5:2.5 and the N/P is 9.

TABLE 15
Composition of N/P ratio optimized LNPs.

| Lipids Molar Ratio (I:H:C:P1:P2) | mRNA | Antibody | N/P Ratio |
|---|---|---|---|
| 25:16:56.5:2.4:0.1 | EGFP | MX1500 | 9 |
| 25:16:56.5:2.4:0.1 | EGFP | MX1500 | 7 |
| 25:16:56.5:2.4:0.1 | EGFP | MX1500 | 4.5 |

Physicochemical characteristics of N/P ratio optimized LNPs of the present disclosure are summarized the table below.

TABLE 16
Physicochemical characteristics of N/P ratio optimized LNPs.

| | | Polydispersity | | mRNA Encapsulation | mRNA Recovery |
|---|---|---|---|---|---|
| LNP | Size (nm) | Index (PI) | Charge (mV) | Efficiency (%) | Rate (%) |
| Lipid10_25_0.1_NP9 | 67 | 0.04 | −2.86 | 97 | 29 |
| Lipid10_25_0.1_NP9_MX1500 | 94 | 0.01 | −2.93 | 94 | 45 |
| Lipid10_25_0.1_NP7 | 69 | 0.04 | −4.5 | 97 | 27 |
| Lipid10_25_0.1_NP7_MX1500 | 94 | 0.03 | −3.05 | 93 | 55 |
| Lipid10_25_0.1_NP4.5 | 63 | 0.04 | −1.83 | 76 | 20 |
| Lipid10_25_0.1_NP4.5_MX1500 | 84 | 0.05 | −2.34 | 89 | 73 |

In some aspects, the N/P ratio optimized LNP is an LNP intermediate having a size between about 63 nm and about 67 nm. In some aspects, the N/P ratio optimized LNP is an antibody-conjugated LNP (e.g., a LNP intermediate conjugated to a bispecific antiCD3/antiCD28 antibody, e.g., an MSTAR such as MX1500) having a size between about 84 nm and about 94 nm. In some aspects, the N/P ratio optimized LNP is an LNP intermediate having a polydispersity index of about 0.04. In some aspects, the N/P ratio optimized LNP is an antibody-conjugated LNP (e.g., a LNP intermediate conjugated to bispecific antiCD3/antiCD28 antibody, e.g., an MSTAR such as MX1500) having a polydispersity index between about 0.01 and about 0.05. In some aspects, the N/P ratio optimized LNP is an LNP intermediate having a charge between about −4.5 mV and about −1.83 mV. In some aspects, the N/P ratio optimized LNP is an antibody-conjugated LNP (e.g., a LNP intermediate conjugated to a bispecific antiCD3/antiCD28 antibody, e.g., an MSTAR such as MX1500) having a charge between about −2.34 mV and about −3.05 mV. In some aspects, the N/P ratio optimized LNP is an LNP intermediate having an mRNA encapsulation efficiency between about 76% and about 97%. In some aspects, the N/P ratio optimized LNP is an antibody-conjugated LNP (e.g., a LNP intermediate conjugated to a bispecific antiCD3/antiCD28 antibody, e.g., an MSTAR such as MX1500) having an mRNA encapsulation efficiency between about 89% and about 94%. In some aspects, the N/P ratio optimized LNP is an LNP intermediate having an mRNA recovery rate between about 20% and about 29%. In some aspects, the N/P ratio optimized LNP is an antibody-conjugated LNP (e.g., a LNP intermediate conjugated to a bispecific antiCD3/antiCD28 antibody, e.g., an MSTAR such as MX1500) having an mRNA recovery rate between about 45% and about 73%.

N/P 9 and N/P 7 show very similar mRNA delivery potency and T cell activation profiles. Accordingly, in some aspects, the LNP has an N/P ratio of about 7. In some aspects, the LNP has an N/P ratio of about 9.

### Lipid 10-Containing Optimized LNP Formulations

The present disclosure provides Lipid 10-containing LNPs optimized in multiple ways as disclosed above in detail. Thus, in some aspects, the present disclosure provides optimized Lipid 10-containing LNP formulations for conjugation, e.g., to a bispecific antiCD3/antiCD28 antibody, e.g., an MSTAR such as MX1500. In some aspects the present disclosure provides optimized Lipid 10-containing LNP formulations comprising, e.g., a bispecific antiCD3/antiCD28 antibody, e.g., an MSTAR such as MX1500, attached to the surface of the LNP. In some aspects, the % of molar ratios in these Lipid 10-containing optimized LNPs are: Lipid 10 (25%):DSPC (16%):Cholesterol (56.5%):PEG2000-DMG (2.45%):DSPE-PEG2000-maleimide (0.05%), and the N/P is about 7.

In some aspects, the LNPs of the present disclosure have (i) a molar ratio of ionizable cationic lipid below 50%, below 45%, below 40%, or below 35%, and/or (ii) a molar ratio of cholesterol above 45%, about 50%, or above 55%. In some aspects, the LNPs of the present disclosure have a molar ratio of ionizable cationic lipid below 50%, e.g., about 25%. In some aspects, the LNPs of the present disclosure have a molar ratio of ionizable cationic lipid below 35%, e.g., about 25%. In some aspects, the LNPs of the present disclosure have a molar ratio of cholesterol above 43.5%, e.g., about 56.5%. In some aspects, the LNPs of the present disclosure have a molar ratio of cholesterol above 46.5%, e.g., about 56.5%. In some aspects, the LNPs of the present disclosure have a molar ratio of PEG2000-DMG above 1.5%, e.g., 2.45%.

In some aspects, the composition of the LNP is: Lipid 10 27.5%, DSPC 16%, cholesterol 54%, DMG-PEG2000 2.45%, and DSPE-PEG2000-maleimide 0.05%. In some aspects, the composition of the LNP is: Lipid 10 about 27.5%, DSPC about 16%, cholesterol about 54%, DMG-PEG2000 about 2.45%, and DSPE-PEG2000-maleimide about 0.05%. These LNP can be conjugated to an antibody (e.g., a bispecific antiCD3/CD28 targeting antibody) or two a pair of antibodies (e.g., an antiCD3 antibody and an antiCD238 antibody). Thus, the resulting LNP would be Lipid 10 27.5%, DSPC 16%, cholesterol 54%, DMG-PEG2000 2.45%, DSPE-PEG2000-maleimide-antibody 0.05%. In some aspects, the LNP is conjugated, i.e., the LNP comprising Lipid 10 27.5%, DSPC 16%, cholesterol 54.2%, DMG-PEG2000 2.45%, and DSPE-PEG2000-maleimide 0.05% is an intermediate for the production of an LNP comprising targeting antibodies on its surface, e.g., Lipid 10 27.5%, DSPC 16%, cholesterol 54%, DMG-PEG2000 2.45%, DSPE-PEG2000-maleimide-antiCD3/antiCD28 0.05%.

In some aspects, the composition of the LNP (e.g., an unconjugated LNP) is: Lipid 10 or Lipid 8 about 27.5%, helper lipid (e.g., DSPC) about 16%, structural lipid (e.g, cholesterol) about 54%, PEG-lipid (e.g., DMG-PEG2000) about 2.45%, and chemically modified lipid (e.g., maleimide-containing lipid such as DSPE-PEG2000-maleimide) about 0.05%. In some aspects, the composition of the LNP (e.g., a conjugated LNP) is: Lipid 10 or Lipid 8 about 27.5%, helper lipid (e.g., DSPC) about 16%, structural lipid (e.g, cholesterol) about 54%, PEG-lipid (e.g., DMG-PEG2000) about 2.45%, and chemically modified lipid attached to a targeting moiety (e.g., maleimide-containing lipid such as DSPE-PEG2000-maleimide covalently attached to an anti-CD3/antiCD28 to yield DSPE-PEG2000-maleimide-antiCD3/antiCD28) about 0.05%.

### Method of Production of LNP Targeting T Cells or B Cells:

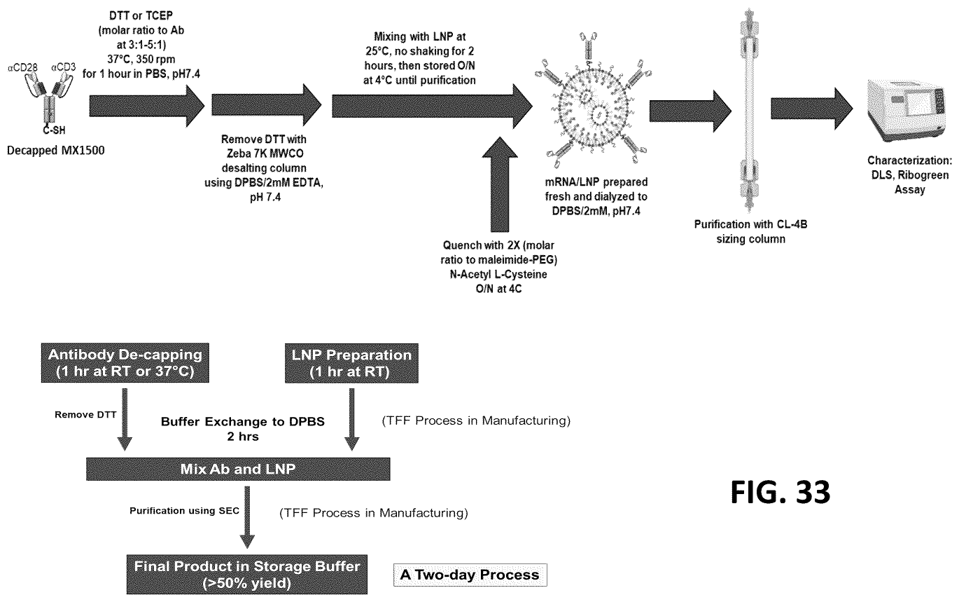

The present disclosure also provides methods to produce a LNP comprising an antibody or combination thereof targeting T cells, e.g., a bispecific antiCD3/antiCD28 antibody, e.g., an MSTAR such as MX1500, or B cells. The general manufacturing process is described in FIG. 33. In some aspects, the method to produce the antibody-conjugated LNPs of the present disclosure comprises (i) decapping the antibody or combination thereof, (ii) preparing a population of optimized LNPs, comprising, e.g., Lipid 10 (27.5%):DSPC (16%):Cholesterol (54%):PEG2000-DMG (2.45%):DSPE-PEG2000-maleimide (0.05%), wherein the LNP comprises a nucleic acid, e.g., a mRNA, at a N/P of about 7; (iii) admixing the antibody or combination thereof of step (i) with the optimized LNPs of step (ii); (iv) purifying the antibody-conjugated LNP, e.g., using SEC; and, (v) storing the antibody-conjugated LNP in a storage buffer. In some aspects, decapping is conducted by using DTT. In some aspects, the manufacture process comprises the use of tangential flow filtration (TFF). In some aspects, TFF is used to remove excess lipids, non-encapsulated RNA, and impurities between step (ii) and step (iii). In some aspects, TFF is used to remove unconjugated antibodies and impurities between step (iii) and step (iv). In some aspects, the compositions obtained after steps (i) and/or (ii) are buffer exchanged. In some aspects, the compositions obtained after steps (i) and/or (ii) are buffer exchanged in DPBS (Dulbecco's Phosphate Buffer Saline), e.g., during 2 hours.

### Optimized LNPs for CAR Delivery

The present disclosure provides a targeted delivery system comprising
(a) an LNP or combination thereof comprising a first antibody (e.g., an anti-CD3 antibody) or antigen-binding portion thereof anchored to the surface of the LNP and a payload encapsulated in the LNP, wherein:
   (i) the first antibody or antigen-binding portion thereof specifically binds to a first target molecule on the surface of a T cell (e.g., CD3) and,
   (ii) the binding of the first antibody or antigen-binding portion thereof to the first target molecule induces T cell uptake of the payload, and
(b) a costimulator (e.g., an anti-CD28 antibody) that specifically binds to a second target molecule on the surface of the T cell (e.g., CD28), wherein
   (i) the binding of the costimulator to the second target molecule increases expression of the payload, and
   (ii) the costimulator is selected from the group consisting of
      (z1) a second specificity of the first antibody, wherein the second specificity targets the second target molecule (e.g., an anti-CD28 portion of an anti-CD3/anti-CD28 bispecific antibody);
      (z2) a second antibody or antigen-binding portion thereof against the second target molecule (e.g., an anti-CD28 antibody), wherein the second antibody or antigen-binding portion thereof is located on the surface of the LNP; and,
      (z3) a second antibody or antigen-binding portion thereof against the second target molecule, wherein the second antibody or antigen-binding portion thereof is located on the surface of a second LNP.

The present disclosure provides a targeted delivery system comprising an LNP comprising
(i) a first antibody (e.g., an anti-CD3 antibody) or antigen-binding portion thereof anchored to the surface of the LNP and a payload encapsulated in the LNP, wherein the first antibody or antigen-binding portion thereof specifically binds to a first target molecule on the surface of a T cell (e.g., CD3) and, wherein the binding of the first antibody or antigen-binding portion thereof to the first target molecule induces T cell uptake of the payload, and
(ii) a costimulator (e.g., an anti-CD28 antibody) that specifically binds to a second target molecule on the surface of the T cell (e.g., CD28), wherein the binding of the costimulator to the second target molecule increases expression of the payload.

The present disclosure provides a targeted delivery system comprising an LNP comprising a bispecific anti-CD3/anti-CD28 antibody anchored to the surface of the LNP and a payload encapsulated in the LNP, wherein
(i) the anti-CD3 portion of the bispecific antibody binds to CD3 on the surface of a T cell;
(ii) the binding of the anti-CD3 portion of the bispecific antibody to CD3 induces T cell uptake of the payload;
(iii) the anti-CD28 portion of the bispecific antibody is a costimulator that specifically binds to a CD28 on the surface of the T cell; and,
(iv) the binding of the anti-CD28 portion of the bispecific antibody to CD28 molecule increases expression of the payload.

In some aspects, the LNP further comprises anti-CD2 and/or anti-CD8 antibodies covalently attached to the surface of the LNP. In some aspects, the covalent attachment of anti-CD3 monospecific antibodies, anti-CD28 monospecific antibodies, bispecific anti-CD3/anti-CD28 antibodies, anti-CD2 antibodies, or anti-CD8 antibodies to the surface of the LNP is conducted using maleimide chemistry as described in the present disclosure.

In some aspects, one or more of the antibodies attached to the surface of an LNP of the present disclosure has the topology VL-CL-Linker-VH-CH1-Fc, wherein VL is a light chain variable region, CL is a light chain constant region, VH is a heavy chain variable regions, CH1 is a heavy chain constant domain 1, and Fc is an Fc domain.

In some aspects, the bispecific anti-CD3/anti-CD28 is MX1500. In some aspects, the bispecific anti-CD3/anti-CD28 is MX1243.

In some aspects, the LNP comprises about 27.5 mol % of an ionizable cationic lipid (iLipid). In some aspects, the LNP comprises 27.5 mol % of iLipid; 16 mol % of DSPC; 2.45 mol % DMG-PEG2000; 0.05 mol % DSPE-PEG2000-maleimide (which is covalently linked to anti-CD3, anti-CD28, bispecific anti-CD3/CD28, anti-CD8, anti-CD2, or a combination thereof following conjugation); and 54 mol % of cholesterol. In some aspects, the iLipid is selected from cKK-E12, MC3, SM-102, ACL-0315, KC2, Lipid A6, Lipid M, Lipid 10, C14-4, any one of MDX1-MDX13, and any other iLipid disclosed herein. In some aspects, the iLipid is cKK-E12. In some aspects, the iLipid is MC3. In some aspects, the iLipid is SM-102. In some aspects, the iLipid is ACL-0315. In some aspects, the iLipid is KC2. In some aspects, the iLipid is Lipid A6. In some aspects, the iLipid is Lipid M. In some aspects, the iLipid is Lipid 10. In some aspects, the iLipid is C14-4.

In some aspects, the payload comprises an mRNA encoding an anti-CD20 CAR. In some aspects, the payload comprises an mRNA encoding an anti-CD19 CAR. In some aspects, the payload comprises an anti-CD79b CAR. In some aspects, the payload comprises a CAR selected from the group consisting of an anti-CD19 CAR, an anti-CD20 CAR, and anti-CD79b CAR, or a combination thereof. In some aspects, the payload comprises a CAR selected from the group consisting of an anti-CD19 second, third, fourth or fifth generation CAR, an anti-CD20 second, third, fourth or fifth generation CAR, and anti-CD79b second, third, fourth or fifth generation CAR, or a combination thereof.

In some aspects, the anti-CD20 CAR is an RN105 CAR (SEQ ID NO: 542). In some aspects, the anti-CD20 CAR is an RN105 CAR encoded by an mRNA of SEQ ID NO: 543. In some aspects, the anti-CD20 CAR is a CAR comprising the extracellular antigen recognition domain of a CAR of SEQ ID NO: 542. In some aspects, the anti-CD20 CAR is a second, third, fourth or fifth generation CAR comprising the extracellular antigen recognition domain of a CAR of SEQ ID NO: 542. In some aspects, the RN105 CAR is encapsulated in an unconjugated LNP. In some aspects, the RN105 CAR is encapsulated in a conjugated LNP, e.g., an LNP comprising a surface anchored T cell targeting molecule that specifically binds to a T cell specific surface protein. In some aspects, the RN105 CAR is encapsulated in a conjugated LNP wherein the surface anchored T cell targeting molecule is an anti-CD3, anti-CD28, anti-CD2, or anti-CD8 antibody or a combination thereof. In some aspects, the RN105 CAR is encapsulated in a conjugated LNP wherein the surface anchored T cell targeting molecule is a bispecific antibody. In some aspects, the RN105 CAR is encapsulated in a conjugated LNP wherein the surface anchored T cell targeting molecule is a bispecific anti-CD3/anti-CD28 antibody, e.g., MX1243 or MX1500. In some aspects, the RN105 CAR is encapsulated in a conjugated LNP wherein the surface anchored T cell targeting molecule is a bispecific anti-CD3/anti-CD28 antibody comprising two polypeptide chains according to the formula VL-CL-Linker-VH-CH1-Fc. In some aspects, the two polypeptide chains of formula VL-CL-Linker-VH-CH1-Fc comprise a set of mutations that promote heterodimerization, e.g., “knob” or “hole” mutations. In some aspects, unconjugated or conjugated LNP comprising the mRNA encoding RN105 contains a molar percentage of ionizable lipid (ilipid) between about 35% and 50% ilipid or between about 25% and about 35% ilipid, e.g., an ilipid presented in FIG. 31, 47, or 48. In some aspects, the RN105 CAR is encapsulated in a conjugated LNP wherein the surface anchored T cell targeting molecules are a monospecific anti-CD3 antibody (e.g., MX1507) and a monospecific anti-CD28 antibody (e.g., MX1506).

In some aspects, the anti-CD79b CAR is an RN111 CAR (SEQ ID NO: 545). In some aspects, the anti-CD79b CAR is an RN111 CAR encoded by an mRNA of SEQ ID NO: 546. In some aspects, the anti-CD79b CAR is a CAR comprising the extracellular antigen recognition domain of a CAR of SEQ ID NO: 545. In some aspects, the anti-CD79b CAR is a second, third, fourth or fifth generation CAR comprising the extracellular antigen recognition domain of a CAR of SEQ ID NO: 545. In some aspects, the RN111 CAR is encapsulated in an unconjugated LNP. In some aspects, the RN111 CAR is encapsulated in a conjugated LNP, e.g., an LNP comprising a surface anchored T cell targeting molecule that specifically binds to a T cell specific surface protein. In some aspects, the RN111 CAR is encapsulated in a conjugated LNP wherein the surface anchored T cell targeting molecule is an anti-CD3, anti-CD28, anti-CD2, or anti-CD8 antibody or a combination thereof. In some aspects, the RN111 CAR is encapsulated in a conjugated LNP wherein the surface anchored T cell targeting molecule is a bispecific antibody. In some aspects, the RN111 CAR is encapsulated in a conjugated LNP wherein the surface anchored T cell targeting molecule is a bispecific anti-CD3/anti-CD28 antibody, e.g., MX1243 or MX1500. In some aspects, the RN111 CAR is encapsulated in a conjugated LNP wherein the surface anchored T cell targeting molecule is a bispecific anti-CD3/anti-CD28 antibody comprising two polypeptide chains according to the formula VL-CL-Linker-VH-CH1-Fc. In some aspects, the two polypeptide chains of formula VL-CL-Linker-VH-CH1-Fc comprise a set of mutations that promote heterodimerization, e.g., “knob” or “hole” mutations. In some aspects, unconjugated or conjugated LNP comprising the mRNA encoding RN111 contains a molar percentage of ionizable lipid (ilipid) between about 35% and 50% ilipid or between about 25% and about 35% ilipid, e.g., an ilipid presented in FIG. 31, 47, or 48. In some aspects, the RN111 CAR is encapsulated in a conjugated LNP wherein the surface anchored T cell targeting molecules are a monospecific anti-CD3 antibody (e.g., MX1507) and a monospecific anti-CD28 antibody (e.g., MX1506).

In some aspects, the anti-CD19 CAR is an RN068 CAR (SEQ ID NO: 548). In some aspects, the anti-CD19 CAR is an RN068 CAR encoded by an mRNA of SEQ ID NO: 549. In some aspects, the anti-CD19 CAR is a CAR comprising the extracellular antigen recognition domain of a CAR of SEQ ID NO: 548. In some aspects, the anti-CD19 CAR is a second, third, fourth or fifth generation CAR comprising the extracellular antigen recognition domain of a CAR of SEQ ID NO: 548. In some aspects, the RN068 CAR is encapsulated in an unconjugated LNP. In some aspects, the RN068 CAR is encapsulated in a conjugated LNP, e.g., an LNP comprising a surface anchored T cell targeting molecule that specifically binds to a T cell specific surface protein. In some aspects, the RN068 CAR is encapsulated in a conjugated LNP wherein the surface anchored T cell targeting molecule is an anti-CD3, anti-CD28, anti-CD2, or anti-CD8 antibody or a combination thereof. In some aspects, the RN068 CAR is encapsulated in a conjugated LNP wherein the surface anchored T cell targeting molecule is a bispecific antibody. In some aspects, the RN068 CAR is encapsulated in a conjugated LNP wherein the surface anchored T cell targeting molecule is a bispecific anti-CD3/anti-CD28 antibody, e.g., MX1243 or MX1500. In some aspects, the RN068 CAR is encapsulated in a conjugated LNP wherein the surface anchored T cell targeting molecule is a bispecific anti-CD3/anti-CD28 antibody comprising two polypeptide chains according to the formula VL-CL-Linker-VH-CH1-Fc. In some aspects, the two polypeptide chains of formula VL-CL-Linker-VH-CH1-Fc comprise a set of mutations that promote heterodimerization, e.g., “knob” or “hole” mutations. In some aspects, unconjugated or conjugated LNP comprising the mRNA encoding RN068 contains a molar percentage of ionizable lipid (ilipid) between about 35% and 50% ilipid or between about 25% and about 35% ilipid, e.g., an ilipid presented in FIG. 31, 47, or 48. In some aspects, the RN068 CAR is encapsulated in a conjugated LNP wherein the surface anchored T cell targeting molecules are a monospecific anti-CD3 antibody (e.g., MX1507) and a monospecific anti-CD28 antibody (e.g., MX1506).

In some aspects, the anti-CD19 is an RN082 CAR (SEQ ID NO: 551). In some aspects, the anti-CD19 CAR is an RN082 CAR encoded by an mRNA of SEQ ID NO: 552. In some aspects, the anti-CD19 CAR is a CAR comprising the extracellular antigen recognition domain of a CAR of SEQ ID NO: 551. In some aspects, the anti-CD19 CAR is a second, third, fourth or fifth generation CAR comprising the extracellular antigen recognition domain of a CAR of SEQ ID NO: 551. In some aspects, the RN082 CAR is encapsulated in an unconjugated LNP. In some aspects, the RN082 CAR is encapsulated in a conjugated LNP, e.g., an LNP comprising a surface anchored T cell targeting molecule that specifically binds to a T cell specific surface protein. In some aspects, the RN082 CAR is encapsulated in a conjugated LNP wherein the surface anchored T cell targeting molecule is an anti-CD3, anti-CD28, anti-CD2, or anti-CD8 antibody or a combination thereof. In some aspects, the RN082 CAR is encapsulated in a conjugated LNP wherein the surface anchored T cell targeting molecule is a bispecific antibody. In some aspects, the RN082 CAR is encapsulated in a conjugated LNP wherein the surface anchored T cell targeting molecule is a bispecific anti-CD3/anti-CD28 antibody, e.g., MX1243 or MX1500. In some aspects, the RN082 CAR is encapsulated in a conjugated LNP wherein the surface anchored T cell targeting molecule is a bispecific anti-CD3/anti-CD28 antibody comprising two polypeptide chains according to the formula VL-CL-Linker-VH-CH1-Fc. In some aspects, the two polypeptide chains of formula VL-CL-Linker-VH-CH1-Fc comprise a set of mutations that promote heterodimerization, e.g., “knob” or “hole” mutations. In some aspects, unconjugated or conjugated LNP comprising the mRNA encoding RN082 contains a molar percentage of ionizable lipid (ilipid) between about 35% and 50% ilipid or between about 25% and about 35% ilipid, e.g., an ilipid presented in FIG. 31, 47, or 48. In some aspects, the RN082 CAR is encapsulated in a conjugated LNP wherein the surface anchored T cell targeting molecules are a monospecific anti-CD3 antibody (e.g., MX1507) and a monospecific anti-CD28 antibody (e.g., MX1506).

In some aspects, the anti-CD19 is an RN083 CAR (SEQ ID NO: 554). In some aspects, the anti-CD19 CAR is an RN083 CAR encoded by an mRNA of SEQ ID NO: 555. In some aspects, the anti-CD19 CAR is a CAR comprising the extracellular antigen recognition domain of a CAR of SEQ ID NO: 554. In some aspects, the anti-CD19 CAR is a second, third, fourth or fifth generation CAR comprising the extracellular antigen recognition domain of a CAR of SEQ ID NO: 554. In some aspects, the RN083 CAR is encapsulated in an unconjugated LNP. In some aspects, the RN083 CAR is encapsulated in a conjugated LNP, e.g., an LNP comprising a surface anchored T cell targeting molecule that specifically binds to a T cell specific surface protein. In some aspects, the RN083 CAR is encapsulated in a conjugated LNP wherein the surface anchored T cell targeting molecule is an anti-CD3, anti-CD28, anti-CD2, or anti-CD8 antibody or a combination thereof. In some aspects, the RN083 CAR is encapsulated in a conjugated LNP wherein the surface anchored T cell targeting molecule is a bispecific antibody. In some aspects, the RN083 CAR is encapsulated in a conjugated LNP wherein the surface anchored T cell targeting molecule is a bispecific anti-CD3/anti-CD28 antibody, e.g., MX1243 or MX1500. In some aspects, the RN083 CAR is encapsulated in a conjugated LNP wherein the surface anchored T cell targeting molecule is a bispecific anti-CD3/anti-CD28 antibody comprising two polypeptide chains according to the formula VL-CL-Linker-VH-CH1-Fc. In some aspects, the two polypeptide chains of formula VL-CL-Linker-VH-CH1-Fc comprise a set of mutations that promote heterodimerization, e.g., “knob” or “hole” mutations. In some aspects, unconjugated or conjugated LNP comprising the mRNA encoding RN083 contains a molar percentage of ionizable lipid (ilipid) between about 35% and 50% ilipid or between about 25% and about 35% ilipid, e.g., an ilipid presented in FIG. 31, 47, or 48. In some aspects, the RN083 CAR is encapsulated in a conjugated LNP wherein the surface anchored T cell targeting molecules are a monospecific anti-CD3 antibody (e.g., MX1507) and a monospecific anti-CD28 antibody (e.g., MX1506).

In some aspects, the anti-CD19 is an RN084 CAR (SEQ ID NO: 557). In some aspects, the anti-CD19 CAR is an RN083 CAR encoded by an mRNA of SEQ ID NO: 558. In some aspects, the anti-CD19 CAR is a CAR comprising the extracellular antigen recognition domain of a CAR of SEQ ID NO: 557. In some aspects, the anti-CD19 CAR is a second, third, fourth or fifth generation CAR comprising the extracellular antigen recognition domain of a CAR of SEQ ID NO: 557. In some aspects, the RN084 CAR is encapsulated in an unconjugated LNP. In some aspects, the RN084 CAR is encapsulated in a conjugated LNP, e.g., an LNP comprising a surface anchored T cell targeting molecule that specifically binds to a T cell specific surface protein. In some aspects, the RN084 CAR is encapsulated in a conjugated LNP wherein the surface anchored T cell targeting molecule is an anti-CD3, anti-CD28, anti-CD2, or anti-CD8 antibody or a combination thereof. In some aspects, the RN084 CAR is encapsulated in a conjugated LNP wherein the surface anchored T cell targeting molecule is a bispecific antibody. In some aspects, the RN084 CAR is encapsulated in a conjugated LNP wherein the surface anchored T cell targeting molecule is a bispecific anti-CD3/anti-CD28 antibody, e.g., MX1243 or MX1500. In some aspects, the RN084 CAR is encapsulated in a conjugated LNP wherein the surface anchored T cell targeting molecule is a bispecific anti-CD3/anti-CD28 antibody comprising two polypeptide chains according to the formula VL-CL-Linker-VH-CH1-Fc. In some aspects, the two polypeptide chains of formula VL-CL-Linker-VH-CH1-Fc comprise a set of mutations that promote heterodimerization, e.g., “knob” or “hole” mutations. In some aspects, unconjugated or conjugated LNP comprising the mRNA encoding RN084 contains a molar percentage of ionizable lipid (ilipid) between about 35% and 50% ilipid or between about 25% and about 35% ilipid, e.g., an ilipid presented in FIG. 31, 47, or 48. In some aspects, the RN084 CAR is encapsulated in a conjugated LNP wherein the surface anchored T cell targeting molecules are a monospecific anti-CD3 antibody (e.g., MX1507) and a monospecific anti-CD28 antibody (e.g., MX1506).

### Aspects

In the text below the term “Ex,” wherein x is an integer, refers to an aspect of the present invention. Thus, e.g., E1 represents Aspect 1.

E1 comprises a targeted delivery system comprising (a) a LNP or combination thereof comprising a first antibody or antigen-binding portion thereof anchored to the surface of the LNP and a payload, wherein: (i) the first antibody or antigen-binding portion thereof specifically binds to a first target molecule on the surface of a T cell or B-cell, and (ii) the binding of the first antibody or antigen-binding portion thereof to the first target molecule induces T cell or B-cell uptake of the payload, and (b) a costimulator that specifically binds to a second target molecule on the surface of the T cell or B-cell, wherein (i) the binding of the costimulator to the second target molecule increases expression of the payload, and (ii) the costimulator is selected from the group consisting of (z1) a second specificity of the first antibody, wherein the second specificity targets the second target molecule; (z2) a second antibody or antigen-binding portion thereof against the second target molecule, wherein the second antibody or antigen-binding portion thereof is located on the surface of the LNP; and, (z3) a second antibody or antigen-binding portion thereof against the second target molecule, wherein the second antibody or antigen-binding portion thereof is located on the surface of a second LNP. E2 comprises a T cell targeted delivery system comprising (a) a LNP comprising a surface anchored T cell targeting molecule that specifically binds to a T cell specific surface protein, and (b) a costimulator of T cell activation. E3 comprises the T cell targeted delivery system of E2, wherein the T cell specific surface protein is CD3. E4 comprises the T cell targeted delivery system of E2, wherein the T cell targeting molecule comprises an anti CD3 antibody or antigen-binding portion thereof. E5 comprises the T cell delivery system of E2, wherein the costimulator of T cell activation is a CD28 agonist. E6 comprises the T cell delivery system of E5, wherein the CD28 agonist is an antibody that specifically binds CD28 or an antigen-binding portion thereof. E7 comprises the T cell delivery system of E5, wherein the CD28 agonist is selected from the group consisting of an antibody that specifically binds CD28, a CD28 ligand, an aptamer, a peptide, a small molecule, or, a combination thereof. E8 comprises the T cell delivery system of E7, wherein the CD28 ligand is B7-1 (CD80), B7-2 (CD86), or a combination thereof. E9 comprises the T cell delivery system of E7, wherein the aptamer is CD28Apt7-dimer comprising a forward sequence of SEQ ID NO: 140), and a reverse sequence of SEQ ID NO: 141.

E10 comprises the T cell delivery system of E2, wherein the costimulator of T cell activation is an agonist of ICOS, B7, CD226, CRTAM, 41-BB, OX40, CD27, GITR, HVEM, CD40, BAFFR, BAFF, or a combination thereof. E11 comprises a T cell targeted delivery system comprising (a) a LNP comprising a surface anchored T cell targeting molecule that specifically binds to one or two T cell specific surface proteins; or, (b) a LNP comprising one or two surface anchored T cell targeting molecules, wherein each T cell targeting molecule specifically binds to one or two T cell specific surface proteins; or, (c) a set of LNP comprising at least two LNPs, wherein the first LNP comprises a first T cell targeting molecule that specifically binds to a first T cell specific surface protein, and the second LNP comprises a second T cell targeting molecule that specifically binds to a second T cell specific surface protein; wherein the delivery system targets at least two T cell specific surface proteins, and wherein the LNP encapsulates a payload. E12 comprises the T cell targeted delivery system of E11, wherein the T cell specific surface proteins comprise CD3, and CD28. E1 comprises the T cell targeted delivery system of E11, wherein the T cell specific surface proteins comprise CD3 and a T cell specific surface protein selected from CD2, CD4, CD5, CD7, CD8, CD28, 4-1BB, NKG2D, or a combination thereof. E14 comprises the T cell targeted delivery system of E11, wherein the T cell specific surface proteins consist of CD3 and CD28, or CD3 and 4-1BB. E15 comprises the T cell targeted delivery system of E11, wherein the LNP comprises (i) a cationic or ionizable cationic lipid or lipidoid; (ii) a structural lipid; (iii) a helper lipid; and, (iv) a stabilizing lipid. E16 comprises the T cell targeted delivery system of E15, wherein the ionizable cationic lipid or lipidoid is selected from the group consisting of cKK-E12, ALC-0315, SM-102, YK-009, DLin-MC3-DMA (MC3), DLin-KC2-DMA (KC2), A6, OF-02, A18-Iso5-2DC18, 98N12-5, 9A1p9, C12-200, 7C1, G0-C14, L319, 304O13, OF-Deg-Lin, 306-O12B, 306O110, FTT5, Lipid 8, Lipid 10, any one of MDX1-MDX13, and combinations thereof. E17 comprises the T cell targeted delivery system of E16, wherein the ionizable cationic lipid or lipidoid is cKK-E12 (3,6-bis(4-(bis(2-hydroxydodecyl)amino)butyl) piperazine-2,5-dione), Lipid 8, or Lipid 10. E18 comprises the T cell targeted delivery system of E15, wherein the cationic lipid is DOTAP or DOTMA. E19 comprises the T cell targeted delivery system of E15, wherein the structural lipid is a sterol.

E20 comprises the T cell targeted delivery system of E19, wherein the sterol is selected from the group consisting of cholesterol, beta-cholesterol, ergosterol, 7-dehydrocholesterol, 24S-hydroxycholesterol, lanosterol, cycloartenol, fucosterol, saringosterol, campesterol, 0-sitosterol, sitostanol, coprostanol, avenasterol, stigmasterol, and any combination thereof. E21 comprises the T cell targeted delivery system of E19, wherein the sterol is cholesterol. E22 comprises the T cell targeted delivery system of E15, wherein the helper lipid comprises at least one symmetric phospholipid, asymmetric lipid, lysolipid, fatty acid, or a combination thereof. E23 comprises the T cell targeted delivery system of E22, wherein the symmetric phospholipid comprises a phosphocholine (PC). E24 comprises the T cell targeted delivery system of E23, wherein the phosphocholine is selected from the group consisting of DSPC, DOPC, DLPV, DMPC, and any combination thereof. E25 comprises the T cell targeted delivery system of E22, wherein the symmetric phospholipid comprises a phosphoethanolamine (PE). E26 comprises the T cell targeted delivery system of E25, wherein the phosphoethanolamine is the selected from the group consisting of DSPE, DOPE, DLPE, DMPE, and a combination thereof. E27 comprises the T cell targeted delivery system of E25, where the stabilizing lipid comprises a PEG-lipid. E28 comprises the T cell targeted delivery system of E27, where the PEG-lipid is a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or a combination thereof. E29 comprises the T cell targeted delivery system of E27, wherein the PEG-lipid is selected from the group consisting of PEG-c-DOMG, PEG-DMG, PEG-DLPE, PEG-DMPE, PEG-DPPC, PEG-DSPE, and any combination thereof.

E30 comprises the T cell targeted delivery system of E25, wherein PEG-lipid is selected from the group consisting of DMG-PEG2000, DSPE-PEG2000, and a combination thereof. E31 comprises the T cell targeted delivery system of E30, wherein the DMG-PEG2000 and/or DSPE-PEG2000 comprises a maleimide group. E32 comprises the T cell targeted delivery system of E15, wherein the cationic or ionizable cationic lipid or lipidoid is at a 30% to 60% molar ratio, the structural lipid is at an about 20% to about 60% molar ratio, and the total phospholipid (helper lipid plus stabilizing lipid) is at about 5% to about 30% molar ratio. E33 comprises the T cell targeted delivery system of E17, wherein the cKK-E12, Lipid 8, or Lipid 10 and a nucleic acid payload are at an about 5% to about 15% weight ratio. E34 comprises the T cell targeted delivery system of E33, wherein the nucleic acid payload is an mRNA. E35 comprises the T cell targeted delivery system of E11, wherein the LNP comprises a molar ratio of about 46.5% cholesterol, about 0.5% DSPE-PEG2000-maleimide, about 16% DOPE, about 35% cKK-E12, Lipid 8, or Lipid 10, and about 2% DMG-PEG2000. E36 comprises the T cell targeted delivery system of any one of E11 to E35, wherein the average hydrodynamic diameter of the LNP is between about 85 nm and about 110 nm. E37 comprises the T cell targeted delivery system of E36, wherein the average hydrodynamic diameter of the LNP is about 95±5 nm. E38 comprises the T cell targeted delivery system of any one of E11 to E37, wherein the encapsulation efficiency of the payload is about 95±5%. E39 comprises the T cell targeted delivery system of E38, wherein the encapsulation efficiency of the payload is about 100%.

E40 comprises the T cell targeted delivery system of any one of E11 to E39, wherein the ratio of payload to T cell targeting molecule (w/w) is between about 1 and about 4. E41 comprises the T cell targeted delivery system of E40, wherein the ratio of payload to T cell targeting molecule (w/w) is about 1.5. E42 comprises the T cell targeted delivery system of any one of E11 to E41, wherein the average number of T cell targeting molecules per LNP is between about 1 and about 6. E43 comprises the T cell targeted delivery system of E42, wherein the average number of T cell targeting molecules per LNP is about 1, about 2, about 3, about 4, about 5 or about 6. E44 comprises the T cell targeted delivery system of any one of E11 to E43, wherein the average number of payload molecules per LNP is between about 1 and about 4. E45 comprises the T cell targeted delivery system of any one of E11 to E44, wherein the average number of payload molecules per LNP is about 2. E46 comprises the T cell targeted delivery system of any one of E11 to E45, wherein the T cell targeting molecule is covalently attached to the LNP via a maleimide moiety. E47 comprises the T cell targeted delivery system of E46, wherein the maleimide moiety if formed by reaction of maleimide group present on to the surface of the LNP and a thiol group present on the T cell targeting molecule. E48 comprises the T cell targeted delivery system of E46, wherein the maleimide moiety if formed by reaction of a maleimide group present on the T cell targeting molecule and a thiol group present on the surface of the LNP. E49 comprises the T cell targeted delivery system of E47, wherein the maleimide group present on to the surface of the LNP is covalently linked to a lipid.

E50 comprises the T cell targeted delivery system of E49, wherein the lipid is a phospholipid. E51 comprises the T cell targeted delivery system of E50, wherein the phospholipid in DSPE. E52 comprises the T cell targeted delivery system of E49, wherein the maleimide group is covalently linked to the lipid via a spacer interposed between the maleimide group and the lipid. E53 comprises the T cell targeted delivery system of E52, wherein the spacer is a water-soluble biopolymer. E54 comprises the T cell targeted delivery system of E53, wherein the water-soluble biopolymer comprises polyethylene glycol (PEG), polyglycerol (PG), or poly(propylene glycol) (PPG). E55 comprises the T cell targeted delivery system of E52, wherein the lipid is a PEG-lipid. E56 comprises the T cell targeted delivery system of E55, wherein the PEG-lipid is DSPE-PEG2000-maleimide. E57 comprises the T cell targeted delivery system of any one of E11 to E56, wherein the payload comprises a polypeptide, a peptide, a polynucleotide, a chemical compound, or any combination thereof. E58 comprises the T cell targeted delivery system of E57, wherein the chemical compound is a small molecule. E59 comprises the T cell targeted delivery system of E58, wherein the small molecule is a proteolysis-targeting chimera (PROTAC).

E60 comprises the T cell targeted delivery system of E58, wherein the small molecule is a nucleotide. E61 comprises the T cell targeted delivery system of E60, wherein the nucleotide is a stimulator of interferon genes protein (STING) agonist. E62 comprises the T cell targeted delivery system of E58, wherein the small molecule is a chemotherapy agent. E63 comprises the T cell targeted delivery system of E57, wherein the polynucleotide is a therapeutic oligonucleotide. E64 comprises the T cell targeted delivery system of E63, wherein the therapeutic oligonucleotide is an antisense oligonucleotide (ASO), a phosphorodiamidate morpholino oligonucleotide (PMO), a siRNA, a miRNA, or a shRNA. E65 comprises the T cell targeted delivery system of E57, wherein the polynucleotide is an mRNA, a plasmid, or a vector. E66 comprises the T cell targeted delivery system of E65, wherein the mRNA comprises at least one nucleotide analogue. E67 comprises the T cell targeted delivery system of E65, wherein the mRNA is codon optimized. E68 comprises the T cell targeted delivery system of E65, wherein the mRNA encodes a chimeric antigen receptor (CAR). E69 comprises the T cell targeted delivery system of E65, wherein the mRNA encodes a component of a gene editing system.

E70 comprises the T cell targeted delivery system of E69, wherein the component of the gene editing system is a gRNA, a nuclease, or a combination thereof. E71 comprises the T cell targeted delivery system of E65, wherein the mRNA encodes an enzyme, receptor, ion channel, vaccine antigen, hormone, cytokine, growth factor, or apoptosis regulator (e.g., an inducer of cell death). E72 comprises the T cell targeted delivery system of E65, wherein the vector is a linear vector. E73 comprises the T cell targeted delivery system of E72, wherein the linear vector is an AAV vector. E74 comprises the T cell targeted delivery system of E57, wherein the polypeptide is an enzyme, a receptor, an ion channel channel, a vaccine antigen, or an antibody or antigen-binding portion thereof. E75 comprises the T cell targeted delivery system of E57, wherein the peptide is a hormone, a cytokine, or a growth factor. E76 comprises the T cell targeted delivery system of E65, wherein the mRNA encodes an antibody or an antigen-binding portion thereof. E77 comprises the T cell targeted delivery system of any one of E11 to E76, wherein the T cell targeting molecule comprises, consists, or consists essentially of an antigen binding polypeptide that specifically binds to a first T cell specific surface protein and to a second T cell specific surface protein, wherein the antigen binding polypeptide has a structure represented by: VL1-VL2-VH2-VH1; VH1-VH2-VL2-VL1; VL1-L1-VL2-L2-VH2-L3-VH1; or VH1-L1-VH2-L2-VL2-L3-VL1; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; and L1, L2 and L3 are amino acid linkers. E78 comprises the T cell targeted delivery system of any one of E11 to E76, wherein the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by: VL1-VL2-VH2-VH1; VH1-VH2-VL2-VL1; VL1-L1-VL2-L2-VH2-L3-VH1; or VH1-L1-VH2-L2-VL2-L3-VL1; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; and L1, L2 and L3 are amino acid linkers. E79 comprises the T cell targeted delivery system of any one of E11 to E76, wherein the T cell targeting molecule comprises, consists, or consists essentially of an antigen binding polypeptide that specifically binds to a first T cell specific surface protein and to a second T cell specific surface protein having a structure represented by: VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH2), an immunoglobulin heavy chain constant region 3 (CH3), and optionally, an immunoglobulin hinge; and L1, L2, L3 and L4 are amino acid linkers.

E80 comprises the T cell targeted delivery system of any one of E11 to E76, wherein the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by: Fc; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH2), an immunoglobulin heavy chain constant region 3 (CH3), and optionally, an immunoglobulin hinge; and L1, L2, L3 and L4 are amino acid linkers. E81 comprises the T cell targeted delivery system of any one of E1I to E76, wherein the T cell targeting molecule comprises, consists, or consists essentially of an antigen binding polypeptide that specifically binds to a first T cell specific surface protein and to a second T cell specific surface protein having a structure represented by: VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; and L1, L2, L3, L4 and L5 are amino acid linkers. E82 comprises the T cell targeted delivery system of any one of E1I to E76, wherein the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by: VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; and L1, L2, L3, L4 and L5 are amino acid linkers. E83 comprises the T cell targeted delivery system of any one of E1 to E76, wherein the T cell targeting molecule comprises, consists, or consists essentially of an antigen binding polypeptide that specifically binds to a first T cell specific surface protein and to a second T cell specific surface protein having a structure represented by: VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers. E84 comprises the T cell targeted delivery system of any one of E11 to E76, wherein the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by: Fc; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers. E85 comprises the T cell targeted delivery system of any one of E11 to E76, wherein the T cell targeting molecule comprises, consists, or consists essentially of an antigen binding polypeptide complex comprising a first polypeptide and a second polypeptide that specifically binds to a first T cell specific surface protein and to a second T cell specific surface protein; wherein the first polypeptide has a structure represented by: VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by: Fc; VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers. E86 comprises the T cell targeted delivery system of any one of E11 to E76, wherein the T cell targeting molecule comprises, consists, or consists essentially of an antigen binding polypeptide or antigen binding polypeptide complex that specifically binds to a first T cell specific surface protein and to a second T cell specific surface protein comprising a polypeptide having a structure represented by: VL1-VL2-VH2-VH1-Fc-Fc; VH1-VH2-VL2-VL1-Fc-Fc; VL1-L1-VL2-L2-VH2-L3-VH1-Fc-Fc; VH1-L1-VH2-L2-VL2-L3-VL1-Fc-Fc; VL1-L1-VL2-L2-VH2-L3-VH1-L4-Fc-Fc; VH1-L1-VH2-L2-VL2-L3-VL1-L4-Fc-Fc; VL1-L1-VL2-L2-VH2-L3-VH1-L4-Fc-L5-Fc; or VH1-L1-VH2-L2-VL2-L3-VL1-L4-Fc-L5-Fc; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; Fc is a region comprising an immunoglobulin heavy chain constant region 2 (CH2), an immunoglobulin heavy chain constant region 3 (CH3), and optionally, an immunoglobulin hinge; and L1, L2, L3, L4 and L5 are amino acid linkers. E87 comprises the T cell targeted delivery system of any one of E11 to E76, wherein the T cell targeting molecule comprises, consists, or consists essentially of an antigen binding polypeptide or antigen binding polypeptide complex that specifically binds to a first T cell specific surface protein and to a second T cell specific surface protein comprising a polypeptide having a structure represented by: VL1-VL2-VH2-VH1-CH3; VH1-VH2-VL2-VL1-CH3; VL1-L1-VL2-L2-VH2-L3-VH1-CH3; VH1-L1-VH2-L2-VL2-L3-VL1-CH3; VL1-L1-VL2-L2-VH2-L3-VH1-L4-CH3; VH1-L1-VH2-L2-VL2-L3-VL1-L4-CH3; VL1-VL2-VH2-VH1-CH3-CH3; VH1-VH2-VL2-VL1-CH3-CH3; VL1-L1-VL2-L2-VH2-L3-VH1-CH3-CH3; VH1-L1-VH2-L2-VL2-L3-VL1-CH3-CH3; VL1-L1-VL2-L2-VH2-L3-VH1-L4-CH3-CH3; VH1-L1-VH2-L2-VL2-L3-VL1-L4-CH3-CH3; VL1-L1-VL2-L2-VH2-L3-VH1-L4-CH3-L5-CH3; or VH1-L1-VH2-L2-VL2-L3-VL1-L4-CH3-L5-CH3; wherein: VL1 is a first immunoglobulin light chain variable region that specifically binds to the first T cell specific surface protein; VL2 is a second immunoglobulin light chain variable region that specifically binds to the second T cell specific surface protein; VH1 is a first immunoglobulin heavy chain variable region that specifically binds to the first T cell specific surface protein; VH2 is a second immunoglobulin heavy chain variable region that specifically binds to the second T cell specific surface protein; CH3 is an immunoglobulin heavy chain constant region 3; and L1, L2, L3, L4 and L5 are amino acid linkers. E88 comprises the T cell targeted delivery system of any one of E77 to E87, wherein one or more of the VH1 comprises an amino acid sequence having at least about 90% sequence identity, at least about 95% sequence identity, or about 100% sequence identity to SEQ ID NO: 40 or SEQ ID NO: 44 and/or one or more of the VH2 comprises an amino acid sequence having at least about 90% sequence identity, at least about 95% sequence identity, or about 100% sequence identity to SEQ ID NO: 42. E89 comprises the T cell targeted delivery system of any one of E77 to E88, wherein one or more of the VL1 comprises an amino acid sequence having at least about 90% sequence identity, at least about 95% sequence identity, or about 100% sequence identity to SEQ ID NO: 41 or SEQ ID NO: 45 and/or one or more of the VL2 comprises an amino acid sequence having at least about 90% sequence identity, at least about 95% sequence identity, or about 100% sequence identity to SEQ ID NO: 43.

E90 comprises the T cell targeted delivery system of any one of E77 to E89, wherein the antigen binding polypeptide or antigen binding polypeptide complex comprises a immunoglobulin hinge comprising or consisting of an upper hinge region, a middle hinge region, a lower hinge region, or a combination thereof. E91 comprises the T cell targeted delivery system of any one of E77 to E90, wherein linkers L1, L2, L3, L4, L5, and/or L6 have a length of from about 1 amino acid to about 50 amino acids. E92 comprises the T cell targeted delivery system of any one of E77 to E91, wherein linkers L1, L2, L3, L4, L5, and/or L6 comprise or consist of the amino acid sequence of G, A, GSS, ASG, SEQ ID NOS: 109-131 or a sequence having at least about 50%, at least about 60%, at least about 70%, at least about 80%, at least about 90%, or at least about 95% sequence identity to any one of SEQ ID NOS: 109-131, or a subsequence thereof. E93 comprises the T cell targeted delivery system of any one of E77 to E92, wherein the antigen binding polypeptide comprises at least one heterodimer-forming modification. E94 comprises the T cell targeted delivery system of E93, wherein the heterodimer-forming modification is selected from the group consisting of knob-into-hole modification (KIH), electrostatic steering modification (ESS), protein A affinity-altering modification, or a combination thereof. E95 comprises the T cell targeted delivery system of any one of E77 to E94, wherein the knob-into-hole modification comprises: (i) knob substitutions of S354C and T366W and hole substitutions of Y349C, T366S, L368A and Y407V; (ii) hole substitutions of L234A, L235A and P239A; (iii) hole substitutions of L234A and L235A; (iv) hole substitutions of M428L and N433S; (v) hole substitutions of M252Y, S254T and T256E; or (vi) a combination thereof, wherein the amino acid numbering is according to the EU numbering scheme. E96 comprises the T cell targeted delivery system of any one of E74 to E96, wherein the antigen binding polypeptide comprises a detectable label. E97 comprises the T cell targeted delivery system of E96, wherein the detectable label is a radioactive label, chemiluminescent label, fluorescent label, enzyme, or peptide tag, or a combination thereof. E98 comprises the T cell targeted delivery system of E97, wherein the peptide tag is a polyhistidine tag consisting of from about 4 to about 10 histidine residues. E99 comprises the T cell targeted delivery system of E98, wherein the polyhistidine tag consists of about 8 histidine residues.

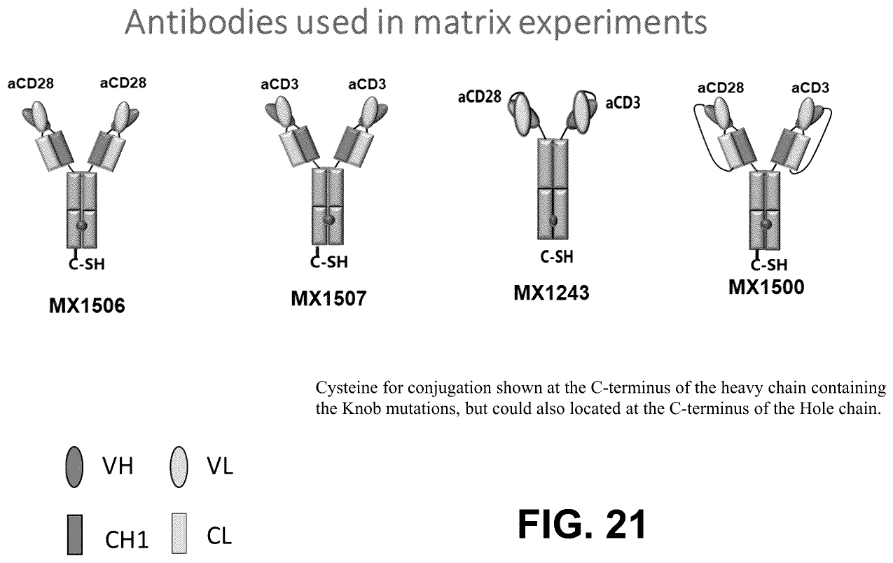

E100 comprises the T cell targeted delivery system of any one of E11 to E99, wherein at least one T cell targeting molecule comprises an antibody. E101 comprises the T cell targeted delivery system of E100, wherein the antibody is IgG, IgM, IgE, IgA or IgD. E102 comprises t the T cell targeted delivery system of E101, wherein the antibody is IgG. E103 comprises the T cell targeted delivery system of E102, wherein the IgG is IgG1, IgG2, IgG3 or IgG4. E104 comprises the T cell targeted delivery system of any one of E11 to E103, wherein at least one T cell targeting molecule comprises a Fab, scFab, Fab′, F(ab′)₂, Fv, or scFv. E105 comprises the T cell targeted delivery system of E101 to E104, wherein the antibody is human or humanized. E106 comprises the T cell targeted delivery system of any one of E11 to E105, wherein the T cell targeting molecule(s) comprise(s): a monospecific monovalent antibody; a monospecific bivalent antibody; a bispecific antibody; or, a combination thereof. E107 comprises the T cell targeted delivery system of any one of E11 to E106, wherein the T cell targeting molecule(s) comprise(s) an antibody or combination thereof selected from the formats presented in FIG. 1A and FIG. 1B. E108 comprises the T cell targeted delivery system of any one of E11 to E106, wherein the T cell targeting molecule(s) comprise(s) an antibody or combination thereof selected from the formats presented in FIG. 21. E109 comprises the T cell targeted delivery system of any one of E11 to E108, wherein the T cell targeting molecule comprises a bispecific anti-CD3/anti-CD28 antibody.

E110 comprises the T cell targeted delivery system of any one of E11 to E108, wherein the T cell targeting molecule(s) comprise(s): (i) a bispecific anti-CD3/anti-CD28 antibody; (ii) a monospecific-monovalent anti-CD3 antibody and a monospecific-monovalent anti-CD28 antibody; (iii) a monospecific-bivalent anti-CD3 antibody and a monospecific-monovalent anti-CD28 antibody; (iv) a monospecific-monovalent anti-CD3 antibody and a monospecific-bivalent anti-CD28 antibody; or, (v) a monospecific-bivalent anti-CD3 antibody and a monospecific-bivalent anti-CD28 antibody. E111 comprises the T cell targeted delivery system of E110, wherein the T cell targeting molecule comprises a bispecific anti-CD3/anti-CD28 antibody selected from Format C, Format D, Format E and Format F of FIG. 1B. E112 comprises the T cell targeted delivery system of any one of E11 to E111, wherein the T cell targeting molecule(s) comprise(s) an anti-CD3 VH region comprising VH-CDR1, VH-CDR2 and VH-CDR3 having at least about 80%, at least about 85%, at least about 90%, at least about 95%, or about 100% sequence identity to SEQ ID NOS: 46, 47 and 48, respectively. E113 comprises the T cell targeted delivery system of any one of E11 to E111, wherein the T cell targeting molecule(s) comprise(s) an anti-CD3 VL region comprising VL-CDR1, VL-CDR2 and VL-CDR3 having at least about 80%, at least about 85%, at least about 90%, at least about 95%, or about 100% sequence identity to SEQ ID NOS: 49, 50 and 51, respectively. E114 comprises the T cell targeted delivery system of any one of E11 to E111, wherein the T cell targeting molecule(s) comprise(s) an anti-CD28 VH region comprising VH-CDR1, VH-CDR2, and VH-CDR3 having at least about 80%, at least about 85%, at least about 90%, at least about 95%, or about 100% sequence identity to SEQ ID NOS: 52, 53, and 54, respectively. E115 comprises the T cell targeted delivery system of any one of E11 to E111, wherein the T cell targeting molecule(s) comprise(s) an anti-CD28 VL region comprising VL-CDR1, VL-CDR2, and VL-CDR3 having at least about 80%, at least about 85%, at least about 90%, at least about 95%, or about 100% sequence identity to SEQ ID NOS: 55, 56, and 57, respectively. E1i6 comprises the T cell targeted delivery system of any one of E11 to E111, wherein the T cell targeting molecule(s) comprise(s) an anti-CD3 VH region comprising VH-CDR1, VH-CDR2 and VH-CDR3 having at least about 80%, at least about 85%, at least about 90%, at least about 95%, or about 100% sequence identity to SEQ ID NOS: 58, 59 and 60, respectively. E117 comprises the T cell targeted delivery system of any one of E11 to E111, wherein the T cell targeting molecule(s) comprise(s) an anti-CD3 VL region comprising VL-CDR1, VL-CDR2 and VL-CDR3 having at least about 80%, at least about 85%, at least about 90%, at least about 95%, or about 100% sequence identity to SEQ ID NOS: 61, 62 and 63, respectively. E118 comprises the T cell targeted delivery system of any one of E11 to E111, wherein the T cell targeting molecule(s) comprise(s) (i) an anti-CD3 VH region comprising VH-CDR1, VH-CDR2 and VH-CDR3 having at least about 80%, at least about 85%, at least about 90%, at least about 95%, or about 100% sequence identity to SEQ ID NOS: 46, 47 and 48, respectively; (ii) an anti-CD3 VL region comprising VL-CDR1, VL-CDR2 and VL-CDR3 having at least about 80%, at least about 85%, at least about 90%, at least about 95%, or about 100% sequence identity to SEQ ID NOS: 49, 50 and 51, respectively; (iii) an anti-CD28 VH region comprising VH-CDR1, VH-CDR2, and VH-CDR3 having at least about 80%, at least about 85%, at least about 90%, at least about 95%, or about 100% sequence identity to SEQ ID NOS: 52, 53, and 54, respectively; (iv) an anti-CD28 VL region comprising VL-CDR1, VL-CDR2, and VL-CDR3 having at least 80%, at least 85%, at least 90%, at least 95%, or 100% identity to SEQ ID NOS: 55, 56, and 57, respectively; (v) an anti-CD3 VH region comprising VH-CDR1, VH-CDR2 and VH-CDR3 having at least 80%, at least 85%, at least 90%, at least 95%, or 100% identity to SEQ ID NOS: 58, 59 and 60, respectively; (vi) an anti-CD3 VL region comprising VL-CDR1, VL-CDR2 and VL-CDR3 having at least about 80%, at least about 85%, at least about 90%, at least about 95%, or 100% sequence identity to SEQ ID NOS: 61, 62 and 63, respectively; or (vii) any combination thereof. E119 comprises the T cell targeted delivery system of any one of E11 to E111, wherein the T cell targeting molecule(s) comprise(s) an anti-CD3 VH region having at least about 80%, at least about 85%, at least about 90%, at least about 95%, or about 100% sequence identity to SEQ ID NO: 40.

E120 comprises the T cell targeted delivery system of any one of E11 to E111, wherein the T cell targeting molecule(s) comprise(s) an anti-CD3 VL region having at least about 80%, at least about 85%, at least about 90%, at least about 95%, or about 100% sequence identity to SEQ ID NO: 41. E121 comprises the T cell targeted delivery system of any one of E11 to E111, wherein the T cell targeting molecule(s) comprise(s) an anti-CD28 VH region having at least about 80%, at least about 85%, at least about 90%, at least about 95%, or about 100% sequence identity to SEQ ID NO: 42. E122 comprises the T cell targeted delivery system of any one of E11 to E111, wherein the T cell targeting molecule(s) comprise(s) an anti-CD28 VL region having at least about 80%, at least about 85%, at least about 90%, at least about 95%, or about 100% sequence identity to SEQ ID NO: 43. E123 comprises the T cell targeted delivery system of any one of E11 to E111, wherein the T cell targeting molecule(s) comprise(s) an anti-CD3 VH region having at least about 80%, at least about 85%, at least about 90%, at least about 95%, or about 100% sequence identity to SEQ ID NO: 44. E124 comprises the T cell targeted delivery system of any one of E11 to E111, wherein the T cell targeting molecule(s) comprise(s) an anti-CD3 VL region having at least about 80%, at least about 85%, at least about 90%, at least about 95%, or about 100% sequence identity to SEQ ID NO: 45. E125 comprises the T cell targeted delivery system of any one of E11 to E111, wherein the T cell targeting molecule(s) comprise(s) (i) an anti-CD3 VH region having at least about 80%, at least about 85%, at least about 90%, at least about 95%, or about 100% sequence identity to SEQ ID NO: 40; (ii) an anti-CD3 VL region having at least about 80%, at least about 85%, at least about 90%, at least about 95%, or about 100% sequence identity to SEQ ID NO: 41; (iii) an anti-CD28 VH region having at least about 80%, at least about 85%, at least about 90%, at least about 95%, or about 100% sequence identity to SEQ ID NO: 42; (iv) an anti-CD28 VL region having at least about 80%, at least about 85%, at least about 90%, at least about 95%, or about 100% sequence identity to SEQ ID NO: 43; (v) an anti-CD3 VH region having at least about 80%, at least about 85%, at least about 90%, at least about 95%, or about 100% sequence identity to SEQ ID NO: 44; (vi) an anti-CD3 VL region having at least about 80%, at least about 85%, at least about 90%, at least about 95%, or about 100% sequence identity to SEQ ID NO: 45; (vii) any combination thereof. E126 comprises the T cell targeted delivery system of any one of E11 to E125, wherein the surface anchored T cell targeting molecule that specifically binds to at least two T cell specific surface proteins comprises a bispecific anti-CD3/anti-CD28 antibody selected from the group consisting of MX1243 (SEQ ID NOS: 18 and 36), MX1501 (SEQ ID NOS: 1 and 19), MX1505 (SEQ ID NOS: 2 and 20), MX1517 (SEQ ID NOS: 3 and 21), MX1518 (SEQ ID NOS: 4 and 22), MX1500 (SEQ ID NOS: 8 and 26), MX1502 (SEQ ID NOS: 9 and 27), MX1516 (SEQ ID NOS: 13 and 31) and MX1520 (SEQ ID NOS: 14 and 32). E127 comprises the T cell targeted delivery system of any one of E11 to E125, wherein the at least two surface anchored T cell targeting molecules are selected from the group consisting of MX1243 (SEQ ID NOS: 18 and 36), MX1501 (SEQ ID NOS: 1 and 19), MX1505 (SEQ ID NOS: 2 and 20), MX1517 (SEQ ID NOS: 3 and 21), MX1518 (SEQ ID NOS: 4 and 22), MX1500 (SEQ ID NOS: 8 and 26), MX1502 (SEQ ID NOS: 9 and 27), MX1516 (SEQ ID NOS: 13 and 31), MX1520 (SEQ ID NOS: 14 and 32), MX1507 (SEQ ID NOS: 5, 23 and 37), MX1519 (SEQ ID NOS: 6, 24, and 38), MX1532 (SEQ ID NOS: 15 and 33), MX1506 (SEQ ID NOS: 7, 25, and 39), MX1503 (SEQ ID NOS: 10 and 28), MX1533 (SEQ ID NOS: 16 and 34), MX1542 (SEQ ID NOS: 17 and 35), and MX1504 (SEQ ID NOS: 11 and 29), wherein at least one of the surface anchored T cell targeting molecules binds to CD3 and at least one of the surface anchored T cell targeting molecules bind to CD28. E128 comprises the T cell targeted delivery system of any one of E11 to E125, wherein the first T cell targeting molecule that specifically binds to a first T cell specific surface protein, and the second T cell targeting molecule that specifically binds to a second T cell specific surface protein in a set of LNP comprising at least two LNPs are selected from the group consisting of MX1243 (SEQ ID NOS: 18 and 36), MX1501 (SEQ ID NOS: 1 and 19), MX1505 (SEQ ID NOS: 2 and 20), MX1517 (SEQ ID NOS: 3 and 21), MX1518 (SEQ ID NOS: 4 and 22), MX1500 (SEQ ID NOS: 8 and 26), MX1502 (SEQ ID NOS: 9 and 27), MX1516 (SEQ ID NOS: 13 and 31), MX1520 (SEQ ID NOS: 14 and 32), MX1507 (SEQ ID NOS: 5, 23, and 37), MX1519 (SEQ ID NOS: 6, 24, and 38), MX1532 (SEQ ID NOS: 15 and 33), MX1506 (SEQ ID NOS: 7, 25, and 39), MX1503 (SEQ ID NOS: 10 and 28), MX1533 (SEQ ID NOS: 16 and 34), MX1542 (SEQ ID NOS: 17 and 35), and MX1504 (SEQ ID NOS: 11 and 29). E129 comprises the T cell targeted delivery system of any one of E11 to E125, wherein the system comprises an anti-CD3/anti-CD28 bispecific antibody selected from the group consisting of MX1243 (SEQ ID NOS: 18 and 36), MX1501 (SEQ ID NOS: 1 and 19), MX1505 (SEQ ID NOS: 2 and 20), MX1517 (SEQ ID NOS: 3 and 21), MX1518 (SEQ ID NOS: 4 and 22), MX1500 (SEQ ID NOS: 8 and 26), MX1502 (SEQ ID NOS: 9 and 27), MX1516 (SEQ ID NOS: 13 and 31), and MX1520 (SEQ ID NOS: 14 and 32).

E130 comprises the T cell targeted delivery system of E129, wherein the system comprises MX1243 (SEQ ID NOS: 18 and 36). E131 comprises the T cell targeted delivery system of any one of E11 to E125, wherein the system comprises an anti-CD3 antibody selected from the group consisting of MX1507 (SEQ ID NOS:5, 23, and 37), MX1519 (SEQ ID NOS: 6, 24, and 38), MX1532 (SEQ ID NOS: 15 and 33), MX1533 (SEQ ID NOS: 16 and 34), MX1542 (SEQ ID NOS: 17 and 35) and an anti-CD28 antibody selected from the group consisting of MX1506 (SEQ ID NOS: 7, 25, and 39), MX1503 (SEQ ID NOS: 10 and 28), and MX1504 (SEQ ID NOS: 11 and 29). E132 comprises the T cell targeted delivery system of any one of E11 to E125, wherein the system comprises at least one antibody selected from the group consisting of MX1243 (SEQ ID NOS: 18 and 36), MX1501 (SEQ ID NOS: 1 and 19), MX1505 (SEQ ID NOS: 2 and 20), MX1517 (SEQ ID NOS: 3 and 21), MX1518 (SEQ ID NOS: 4 and 22), MX1500 (SEQ ID NOS: 8 and 26), MX1502 (SEQ ID NOS: 9 and 27), MX1516 (SEQ ID NOS: 13 and 31), MX1520 (SEQ ID NOS: 14 and 32), MX1507 (SEQ ID NOS: 5, 23, and 37), MX1519 (SEQ ID NOS: 6, 24, and 38), MX1532 (SEQ ID NOS: 15 and 33), MX1506 (SEQ ID NOS: 7 and 25), MX1503 (SEQ ID NOS: 10 and 28), MX1533 (SEQ ID NOS: 16 and 34), MX1542 (SEQ ID NOS: 17 and 35), and MX1504 (SEQ ID NOS: 11 and 29). E133 comprises the T cell targeted delivery system of any one of E11 to E125, wherein the system comprises MX1506 (SEQ ID NOS: 7, 25, and 39) and MX1507 (SEQ ID NOS: 5, 23, and 37). E134 comprises the T cell targeted delivery system of E133, wherein MX1506 (SEQ ID NOS: 7, 25, and 39) and MX1507 (SEQ ID NOS: 5, 23, and 37) are in a single LNP. E135 comprises the T cell targeted delivery system of E133, wherein MX1506 (SEQ ID NOS: 7, 25, and 39) and MX1507 (SEQ ID NOS: 5, 23, and 37) are in separate LNPs. E136 comprises the T cell targeted delivery system of any one of E11 to E135, wherein the LNP comprises a surface ligand that can increase permeation through the blood-brain barrier. E137 comprises the T cell targeted delivery system of E136, wherein the surface ligand is a transferrin receptor. E138 comprises the T cell targeted delivery system of any one of E11 to E137, wherein the LNP comprises a tissue or cell-specific target ligand that increases LNP tropism to a tissue or physiological compartment. E139 comprises the T cell targeted delivery system of any one of E11 to E138, wherein the LNP comprises a surface anchored anti-phagocytic signal.

E140 comprises the T cell targeted delivery system of E139, whereon the anti-phagocytic signal comprises CD47, CD24, a fragment thereof, and any combination thereof. E141 comprises the T cell targeted delivery system of any one of E11 to E140, wherein the LNP comprises a half-live extension moiety. E142 comprises the T cell targeted delivery system of E141, wherein the half-live extension moiety is selected from the group consisting of PEG, albumin, transferrin, and FcRn. E143 comprises a method of manufacturing a targeted delivery system of any one of E1 to E142, comprising conjugating a targeting antibody or combination thereof to a lipid via a maleimide moiety, wherein the lipid integrates into the external surface of the LNP. E144 comprises the method of E143, wherein the targeting antibody is a monospecific antibody. E145 comprises the method of E143, wherein the targeting antibody is a bispecific antibody. E146 comprises the method of E144, wherein a first monospecific targeting antibody is conjugated to a first lipid and a second monospecific targeting antibody is conjugated to a second lipid, thereby yielding a first conjugate and a second conjugate, wherein the first conjugate integrates into the external surface of a first LNP and the second conjugate integrates into the external surface of a second LNP. E147 comprises the method of E146, further comprising combining the first LNP and the second LNP after the conjugation of the first targeting antibody to the surface of a first LNP and the conjugation of the second targeting antibody to the surface of the second LNP. E148 comprises the method of any one of E143 to E147, wherein the conjugation comprises reacting a maleimide group covalently attached to the lipid with a sulfhydryl group of the targeting antibody. E149 comprises the method of E148, wherein the lipid is DSPE-PEG2000-maleimide.

E150 comprises the method of any one of E143 to E149, wherein the conjugation comprises reacting the maleimide group of attached to the targeting antibody with a sulfhydryl group covalently attached to the lipid. E151 comprises the method of E150, wherein the lipid is DSPE-PEG2000—SH. E152 comprises the method of any one of E143 to E151, wherein the conjugate is formed on the LNP. E153 comprises the method of any one of E143 to E151, wherein the conjugate is incorporated into the LNP. E154 comprises a pharmaceutical composition comprising a therapeutic agent encapsulated in the targeted delivery system of any one of E1 to E142 and a pharmaceutically acceptable carrier. E155 comprises a diagnostic composition comprising a diagnostic agent encapsulated in the targeted delivery system or attached to the targeted delivery system of any one of E1 to E142. E156 comprises a kit comprising the targeted delivery system of any one of E1 to E142, the pharmaceutical composition of E154, the diagnostic composition of E155, and instructions for use. E157 comprises a method of treating or preventing a disease or disorder in a subject in need thereof comprising administering a therapeutic agent encapsulated in a targeted delivery system of any one of E1 to E142 to the subject. E158 comprises the method of E157, wherein the disease or disorder is a cancer. E159 comprises the method of E158, wherein the cancer is a tumor.

E160 comprises the method of E158, wherein the cancer is a hematological cancer. E161 comprises the method of E157, wherein the disease or disorder is a genetic disease. E162 comprises the method of E161, wherein the genetic disease is a metabolic disease. E163 comprises the method of E157, whereas the disease or disorder is an inflammatory disease. E164 comprises the method of E157, whereas the disease or disorder is an autoimmune disease. E165 comprises the method of E157, whereas the disease or disorder is an infection. E166 comprises the method of E165, whereas the infection is a viral infection. E167 comprises the method of E165, wherein the infection is a bacterial infection. E168 comprises a gene therapy method comprising administering a therapeutic agent encapsulated in a targeted delivery system of any one of E1 to E142 to a subject. E169 comprises the gene therapy method of E168, wherein the therapeutic agent comprises an mRNA, a vector, or a guide RNA (gRNA).

E170 comprises a CAR-T cell therapy method comprising administering a T cell targeted delivery system of any one of E11 to E142 to the subject, wherein the LNP comprises a CAR or an mRNA encoding a CAR. E171 comprises the CAR-T cell therapy method of E170, wherein the T cell targeted delivery system comprises a vector encoding a CAR protein. E172 comprises the CAR-T cell therapy method of E170, wherein the T cell targeted delivery system comprises an mRNA encoding a CAR protein. E173 comprises the CAR-T cell therapy method of any one of E170 to E172, wherein the vector or mRNA are in the lumen of the LNP. E174 comprises the CAR-T cell therapy method of E170, wherein the T cell targeted delivery system comprises a CAR protein. E175 comprises the CAR-T cell therapy method of E174, wherein the CAR protein is in the lumen of the LNP. E176 comprises the CAR-T cell therapy method of E175, wherein the CAR protein is integrated in the membrane of the LNP. E177 comprises a method to target at least one payload to a cell or tissue comprising (i) encapsulating a payload in a targeted delivery system of any one of E1 to E142; or, (ii) attaching a payload to a targeted delivery system of any one of E1 to E142; or, (iii) a combination thereof. E178 comprises the method of E177, wherein a payload is (i) attached to the inner surface of the LNP; (ii) attached to the outer surface or the LNP; (iii) transverses the lipid layer of the LNP; or, (iv) a combination thereof. E179 comprises a targeted delivery system comprising a LNP comprising an encapsulated payload and a first surface anchored targeting molecule that specifically binds to a first target protein on a target cell or tissue, wherein the binding to the first surface anchored targeting molecule to the first target protein on the target cell or tissue results in (i) delivery of the encapsulated payload to the target cell or tissue, and (ii) activation or inhibition of a biological activity mediated by the first target protein.

E180 comprises the targeted delivery system of E179, further comprising a second surface anchored targeting molecule that specifically binds to a second target protein at the target cell or tissue, wherein the binding of the second surface anchored targeting molecule to the second target protein results in the modulation of the biological function activity mediated by the first target protein. E181 comprises the targeted delivery system of E179 or E180, wherein the cell is a T cell. E182 comprises the targeted delivery system of E179 or E180, wherein the cell is a B cell. E183 comprises the targeted delivery system of any one of E179 to E182, wherein the first target protein is a receptor. E184 comprises the targeted delivery system of any one of E180 to E183, wherein the second target protein is a receptor. E185 comprises the targeted delivery system any one of E179 to E184, wherein the first surface anchored targeting molecule is an agonist, or an antagonist of the first target protein. E186 comprises the targeted delivery system any one of E180 to E185, wherein the second surface anchored targeting molecule is an agonist, or an antagonist of the second target protein. E187 comprises the targeted delivery system of any one of E180 to E186, wherein the second target protein is a costimulator of the first target protein. E188 comprises the targeted delivery system of any one of E180 to E187, wherein the biological function activity is an increase or decrease in protein expression. E189 comprises the targeted delivery system of any one of E180 to E188, wherein the biological function activity is an increase or decrease in cell proliferation.

E190 comprises the targeted delivery system any one of E180 to E189, wherein the first surface anchored targeting molecule and the second surface anchored targeting molecule are part of single molecule. E191 comprises the targeted delivery system E190, wherein the single molecule is a bispecific antibody. E192 comprises the targeted delivery system E179, wherein the single molecule is a bispecific antibody. E193 comprises a LNP composition comprising (i) a cationic or ionizable cationic lipid or lipidoid, wherein the ionizable cationic lipid or lipidoid is selected from the group consisting of cKK-E12, ALC-0315, SM-102, YK-009, DLin-MC3-DMA (MC3), DLin-KC2-DMA (KC2), A6, OF-02, A18-Iso5-2DC18, 98N12-5, 9A1p9, C12-200, 7C1, G0-C14, L319, 304O13, OF-Deg-Lin, 306-O12B, 306O110, FTT5, Lipid 8, Lipid 10, any one of MDX1-MDX13, and combinations thereof; (ii) a sterol; (iii) a helper lipid comprising at least one symmetric phospholipid, asymmetric lipid, lysolipid, fatty acid, or a combination thereof; and, (iv) a stabilizing lipid. E194 comprises the LNP composition of E193, wherein the ionizable cationic lipid or lipidoid is cKK-E12 (3,6-bis(4-(bis(2-hydroxydodecyl)amino)butyl)piperazine-2,5-dione), Lipid 8, or Lipid 10. E195 comprises the LNP composition of E193, wherein the sterol is selected from the group consisting of cholesterol, beta-cholesterol, ergosterol, 7-dehydrocholesterol, 24S-hydroxycholesterol, lanosterol, cycloartenol, fucosterol, saringosterol, campesterol, 0-sitosterol, sitostanol, coprostanol, avenasterol, stigmasterol, and any combination thereof. E196 comprises the LNP composition of E195, wherein the sterol is cholesterol. E197 comprises the LNP composition of E193, wherein the symmetric phospholipid comprises (i) a phosphocholine (PC) selected from the group consisting of DSPC, DOPC, DLPV, DMPC, and any combination thereof, (ii) a phosphoethanolamine (PE) selected from the group consisting of DSPE, DOPE, DLPE, DMPE, and a combination thereof, or, (iii) a combination thereof. E198 comprises the LNP composition of E193, wherein the stabilizing lipid comprises a PEG-lipid selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or a combination thereof. E199 comprises the LNP composition of E198, wherein the PEG-lipid is selected from the group consisting of PEG-c-DOMG, PEG-DMG, PEG-DLPE, PEG-DMPE, PEG-DPPC, PEG-DSPE, and any combination thereof.

E200 comprises the LNP composition of E198, wherein the PEG-lipid is selected from the group consisting of DMG-PEG2000, DSPE-PEG2000, and a combination thereof. E201 comprises the LNP composition of E200, wherein the DMG-PEG2000 and/or DSPE-PEG2000 comprises a maleimide group. E202 comprises the LNP composition of E193, wherein the cationic or ionizable cationic lipid or lipidoid is at a 30% to 60% molar ratio, the structural lipid is at a 20% to 60% molar ratio, and the total phospholipid (helper lipid plus stabilizing lipid) is at 5% to 30% molar ratio. E203 comprises the LNP composition of E193, wherein the LNP composition comprises (i) a molar ratio of about 46.5% cholesterol, about 0.5% DSPE-PEG2000-maleimide, about 16% DOPE, about 35% cKK-E12, and about 2% DMG-PEG2000; or (ii) a molar ratio of about 38.5% cholesterol, a molar ratio of about 10% DSPC, a molar ratio of about 50% Lipid 10, and a molar ratio of about 1.5% DMG-PEG. E204 comprises the LNP composition of any one of E193 to E203, further comprising a payload or a combination thereof. E205 comprises the LNP composition of E204, wherein the LNP encapsulates the payload or combination thereof. E206 comprises the LNP composition of E205, wherein the payload comprises an mRNA or combination thereof. E207 comprises the LNP composition of any one of E204 to E206, wherein the payload is a CAR or a combination thereof. E208 comprises the LNP composition of E207, wherein the CAR is monospecific or bispecific. E209 comprises the LNP composition of E208, wherein the bispecific CAR specifically binds to CD19 and CD20.

E210 comprises a LNP composition comprising (i) an ionizable cationic lipid comprising Lipid 10; (ii) a sterol; (iii) a helper lipid comprising at least one symmetric phospholipid, asymmetric lipid, lysolipid, fatty acid, or a combination thereof, and, (iv) a stabilizing lipid. E211 comprises the LNP composition of E210, wherein the LNP is unconjugated. E212 comprises the LNP composition of E211, wherein the sterol is cholesterol, the helper lipid is DSPC, and the stabilizing lipid is DMG-PEG2000. E213 comprises the LNP composition of E211, comprising 27.5 mol % of Lipid 10; 16 mol % of DSPC; 2.5 mol % of DMG-PEG2000; and 54 mol % of cholesterol. E214 comprises the LNP composition of E210, further comprising a chemically modified lipid. E215 comprises the LNP composition of E214, wherein the chemically modified lipid comprises a maleimide reactive group. E216 comprises the LNP composition of E214 or E215, wherein the chemically modified lipid comprises DSPE-PEG2000-maleimide. E217 comprises the LNP composition of E216, comprising 27.5 mol % of Lipid 10; 16 mol % of DSPC; 2.45 mol % of DMG-PEG2000; 0.05 mol % of DSPE-PEG2000-Maleimide; and 54 mol % of cholesterol. E218 comprises the LNP composition of E217, wherein the LNP is unconjugated. E219 comprises the LNP composition of any one of E214 to E217, wherein the LNP is conjugated.

E220 comprises the LNP composition of E219, wherein the chemically modified lipid is conjugated to a targeting molecule or combination thereof. E221 comprises the LNP composition of E220, wherein the targeting molecule or combination thereof comprises an antibody, antigen-binding portion thereof, or a combination thereof. E222 comprises the LNP composition of E221, wherein the antibody, antigen-binding portion thereof, or combination thereof comprises two monospecific antibodies. E223 comprises the LNP composition of E221, wherein the antibody, antigen-binding portion thereof, or combination thereof comprises a bispecific antibody. E224 comprises the LNP of E223, wherein the bispecific antibody is an MSTAR. E225 comprises the LNP composition of any one of E220 to E224, wherein the targeting molecule or combination thereof targets the LNP to T cells or B cells. E226 comprises the LNP composition of E225, wherein the targeting molecule or combination thereof that targets the LNP to T cells specifically binds to CD3, CD28, or a combination thereof. E227 comprises the LNP composition of E226, wherein the targeting molecule or combination thereof that targets the LNP to B cells specifically binds to CD19, CD79, CD40L, or a combination thereof. E228 comprises the LNP composition of E227, wherein the targeting molecule is a bispecific antibody targeting CD3 and CD28. E229 comprises the LNP composition of E228, wherein the bispecific antibody targeting CD3 and CD28 is MX1500 (SEQ ID NOS: 8 and 26).

E230 comprises the LNP composition of any one of E210 to E229, wherein the LNP encapsulates a payload or a combination thereof. E231 comprises the LNP composition of E230, wherein the payload comprises an mRNA or combination thereof. E232 comprises the LNP composition of E231, wherein the payload comprises one mRNA. E233 comprises the LNP composition of E231, wherein the payload comprises two mRNA. E234 comprises the LNP composition of any one of E231 to E233, wherein the payload is a CAR or a combination thereof. E235 comprises the LNP composition of E232, wherein the one mRNA encodes a monospecific CAR. E236 comprises the LNP composition of E232, where the one mRNA is a bicistronic mRNA encoding two monospecific CAR. E237 comprises the LNP composition of E232, wherein the one mRNA encodes a bispecific CAR. E238 comprises the LNP composition of E237, wherein the bispecific CAR specifically binds to CD19 and CD20. E239 comprises the LNP composition of E233, wherein the two mRNA encode two monospecific CAR.

E240 comprises the LNP composition of E239, wherein a first monospecific CAR specifically binds to CD19 and a second monospecific CAR specifically binds to CD20. E241 comprises the LNP composition of E240, wherein the mRNA encoding the first monospecific CAR is encapsulated in a first LNP and the mRNA encoding the second monospecific CAR is encapsulated in a second LNP. E242 The LNP composition of E240, wherein the mRNA encoding the first monospecific CAR and the mRNA encoding the second monospecific CAR are encapsulated in the same LNP.

### Lipids

The present disclosure also provides a lipid having the structure of any one of MDX1-MDX13 (see FIGS. 57-59). In some aspects, the lipid is MDX1. In some aspects, the lipid is MDX2. In some aspects, the lipid is MDX3. In some aspects, the lipid is MDX4. In some aspects, the lipid is MDX5. In some aspects, the lipid is MDX6. In some aspects, the lipid is MDX7. In some aspects, the lipid is MDX8. In some aspects, the lipid is MDX9. In some aspects, the lipid is MDX10. In some aspects, the lipid is MDX11. In some aspects, the lipid is MDX12. In some aspects, the lipid is MDX13. Such lipids can be used in a lipid nanoparticle (LNP), T cell targeted delivery system, pharmaceutical composition, or method of use, as described herein. MDX1-MDX13 contain a combination of, for example, SM-102 and KC2, and can be made by routine synthetic methods known in the art.

### EXAMPLES

### Example 1

### Determination of Optimal T Cell Targeting by Conjugating CD3 and CD28 Monovalent or Bivalent Antibodies to Lipid Nanoparticles (LNPs)

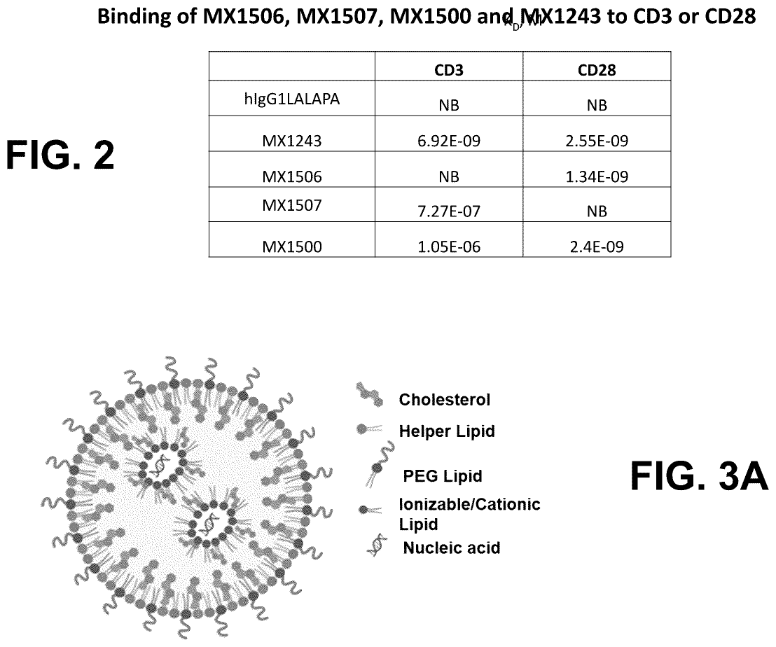

Various monospecific antibody formats were conjugated to LNPs and antibody-LNPs tested for optimal T cell targeting. The antibody formats included monospecific, bivalent and monovalent CD28 antibody formats (FIG. 1A, Format A) and monospecific, bivalent and monovalent CD3 antibody formats (FIG. 1A, Format B). CD28 and CD3 antibodies were conjugated to LNPs at different ratios (FIG. 1A, Table). Binding (K_{D}) of MX1243, MX1506, MX1507 and MX1500 to CD3 and CD28 is presented in FIG. 2.

Further, various bispecific CD28/CD3 antibody formats were conjugated to LNPs. The bispecific CD28/CD3 formats included bispecific CD28/CD3 antibodies (FIG. 1B, Format C), single-arm CD28 scFv/CD3 scFv antibodies (FIG. 1B, Format D), single-arm CD28 scFv/CD3 scFv-Fc molecules (FIG. 1B, Format E), and CD28 scFv/CD3 scFv-Fc mini antibodies (FIG. 1B, Format F).

### Example 2

### LNP Fabrication

Four categories of materials were included in LNPs, such as ionizable/cationic lipid, cholesterol, helper lipid and PEG lipid (FIG. 3A). Different configurations of monospecific and bispecific LNP are presented in FIG. 3B. Ionizable cationic lipids play a key role in LNP formation to interact with nucleic acids. Cholesterol was used to maintain fluidity/stability and enhance the penetration of cell membranes. Helper lipids were used to modulate the structural properties of the LNP; examples of helper lipids were DSPC, DOPC, and DOPE. PEG-lipids could stabilize the LNP by preventing LNP aggregation, interaction with serum proteins, and phagocyte uptake; examples of PEG-lipids were Dimyristoyl PEG 2000, and Dipalmitoyl PEG 5000 (see TABLE 17).

TABLE 17
LNP components

| Phospholipid (Helper) Lipid | Head Group | Tail Group |
|---|---|---|
| DSPE | DS | PE |
| DSPC | DS | PC |
| DOPE | DO | PE |
| DOPC | DO | PC |

Cholesterol
Cholesterol
beta-Cholesterol
PEG lipid
C14 DMG-PEG2000
C14 PE-PEG2000

| Continuous Variables | Lower Limit | Upper Limit |
|---|---|---|
| cKK-E12:mRNA (weight ratio) | 5 | 15 |
| cKK-E12 (molar ratio %) | 30 | 60 |
| Cholesterol (molar ratio %) | 20 | 60 |
| Phospholipid (molar ratio %) | 5 | 30 |

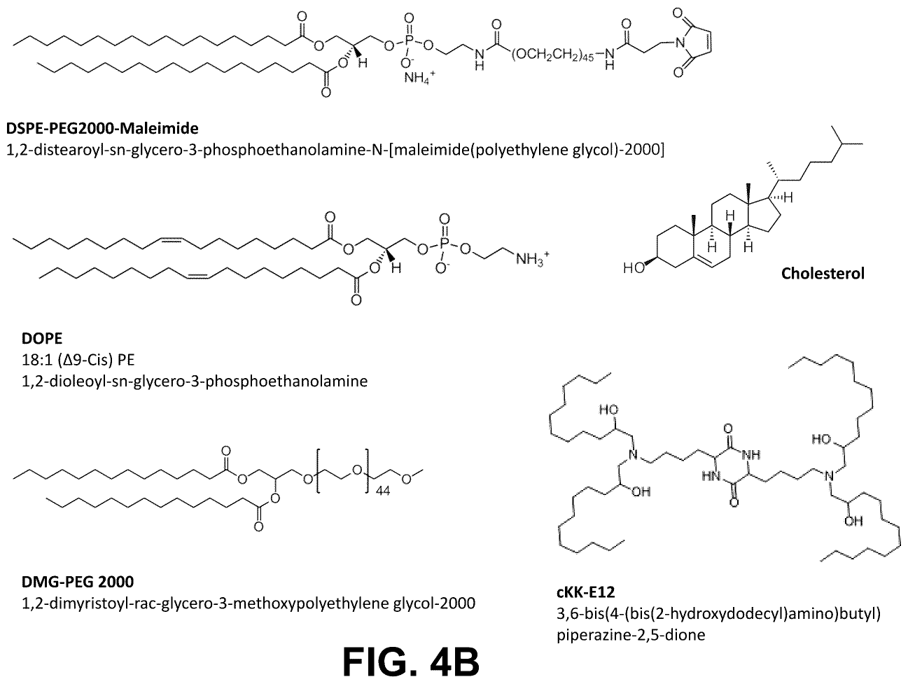

To optimize the LNP formulations, definitive screening design by JMP® 16.2.0 was applied to rapidly identify the lipid components and the composition of LNP (FIG. 4A). In each lipid category, varied materials were used to form LNPs. For example, as helper lipids: DSPE, DSPC, DOPE, or DOPC were used; as ionizable cationic lipids: SM-102, MC3, KC2, ALC-0315, 1-A-N, 11-A-M; as cholesterol: beta-Sitosteril; and as PEG lipid: DMG-PEG1000, 2000, 3000; DSPE-PEG2000-Maleimide were used. These lipid components were used either alone or in combination. (FIG. 4B). Additionally, the molar ratio of each category and the weight ratio of ionizable cationic lipid and nucleic acids were optimized systematically and lower and upper limits, e.g., for weight ratios of ionic lipid cKK-E12 to mRNA, and molar ratios for cKK-E12, cholesterol and phospholipid were determined (TABLE 17).

Lipid, helper lipid, PEG lipid DMG-PEG2000 or PEG lipid with maleimide, and nucleic acids were thawed on ice. One tube of organic phase for lipid and one tube for aqueous phase for nucleic acids were prepared, transferred into syringes, and loaded into a NanoAssemblr (Precision Nanosystems) to prepare the LNPs (FIG. 4A). A dialysis device was used to exchange the buffer of the LNPs and centrifuge and syringe filters were used to sterilize the LNPs. LNPs were stored at 4° C. for characterization and use.

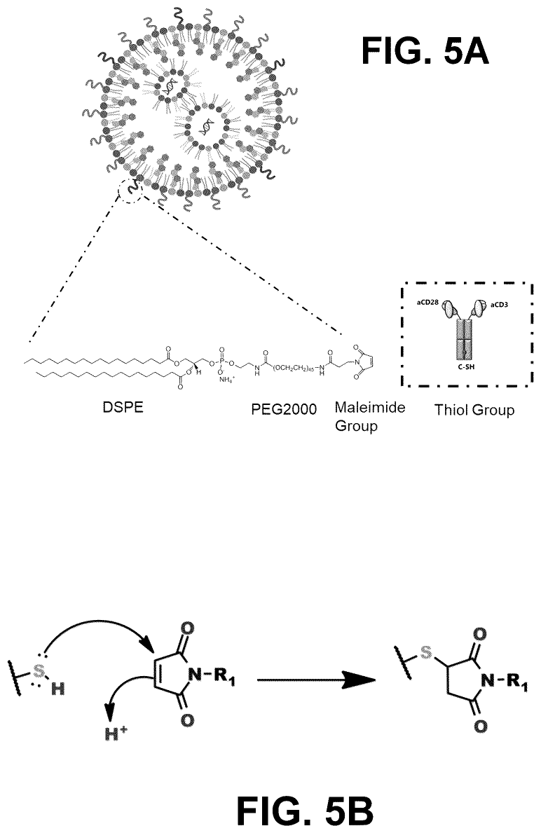

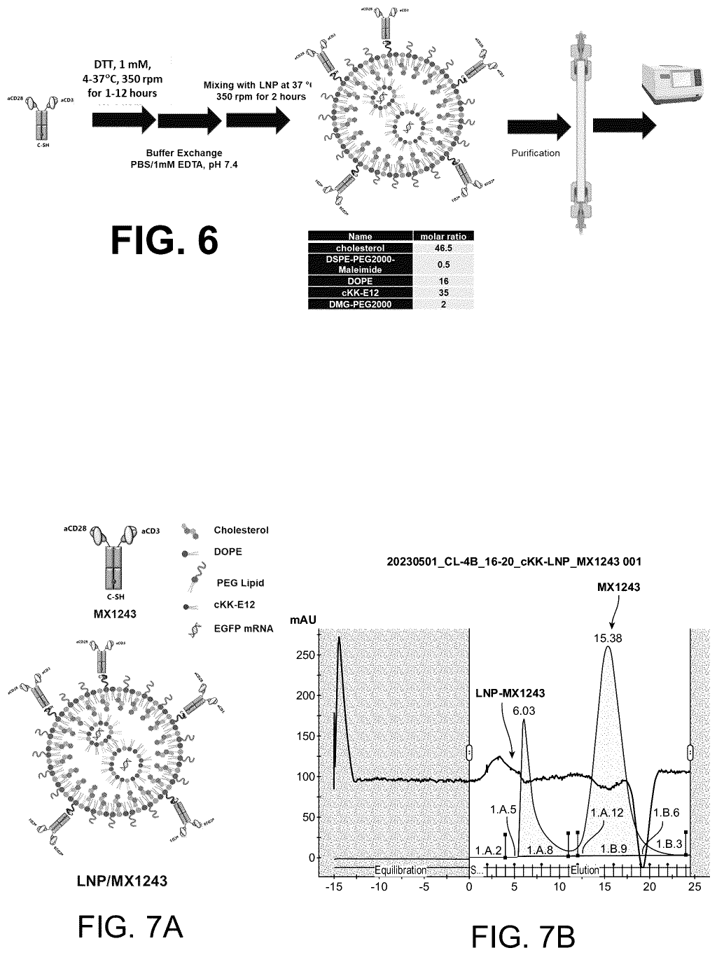

LNP were conjugated to antibody thiol groups by maleimide-thiol reaction (FIG. 5A and FIG. 5B). For conjugation, antibody was incubated with 1 mM DTT at 4-37° C., 350 rpm for 1-12 hours. Buffer was changed to PBS with 1 mM EDTA at pH 7.4 (FIG. 6). The antibody solution was mixed with the LNP solution at 37° C., 350 rpm for 2 hours (FIG. 6). Antibody-conjugated LNPs were purified using Akta Go with CL-4B, crosslinking agarose beads column and concentrated using centrifugal filter devices (FIG. 6). Molar ratios of lipid components of an exemplary LNP are shown in the Table in FIG. 6. An exemplary CD28/CD3 antibody-conjugated, EGFP mRNA encapsulating LNP (LNP/MX1243) is shown in FIG. 7A. Elution peaks for LNP-MX1243 conjugates and free MX1243 antibodies are shown in FIG. 7B.

An ELISA was used to measure antibody-conjugated LNP binding to the target. Briefly, Immulon MaxiSorp 96-well plates were coated with 100 μL of antigen at a concentration of 2 μg/mL in PBS at 4° C. overnight or 37° C. for 2 hours. Following washes with PBST, the plate was blocked with PBST+2% BSA+5% milk for 1 hour at room temperature (RT). Following washes with PBST, the antibody-conjugated LNP was incubated for 1 hour at RT.

The antibody-conjugated LNP concentrations were measured as follows:

Antigen Coating: Immulon MaxiSorp 96-well plates were coated with 100 μl of coating antigen at a concentration of 2 μg/ml in phosphate-buffered saline (PBS), pH 7.4, overnight at 4° C. or at 37° C. for 2 hours. The plate was washed with 300 μl/well PBS-T [PBS+0.05% Tween 20] washing buffer for 5 times.

Blocking: The plates were incubated with 280 μl of blocking buffer (PBS-T+2% BSA+5% milk) for 1 hour at room temperature. The plates were washed with 300 μl of PBS-T washing buffer once.

First antibody binding: Standard protein was serially diluted in dilution buffer (PBS+1% Triton X-100, 2.5% milk+1% BSA), protein conjugated LNP was 200× and 800× fold diluted in dilution buffer. Diluted standard and LNP were added 100 μl to the antigen-coated plates, and incubated 1 hour at room temperature. Plates were then washed with 300 μl of PBS-T washing buffer for 5 times.

Second antibody binding: The plates were incubated with 100 μl anti-human Fc-HRP (Jackson ImmunoResearch Inc #109-035-190 1:5000) in dilution buffer for 1 hour at room temperature. The plates were washed with 300 μl of PBS-T washing buffer for 5 times. Plate development: 100 μl of KPL SureBlue™ TMB 1-Component Microwell Peroxidase Substrate was added in each well for 5-20 minutes at room temperature. The reaction was stopped by adding 100 μl of TMB BlueSTOP™ Solution to each well.

Plate reading and data analysis: The plates were read at 650 nm in 30 minutes by Varioskan Lux (Thermo Scientific). The data was plotted using excel program and the protein concentration was calculated based on the standard curve in GraphPad Prism.

### Example 3

### LNP Quality Control

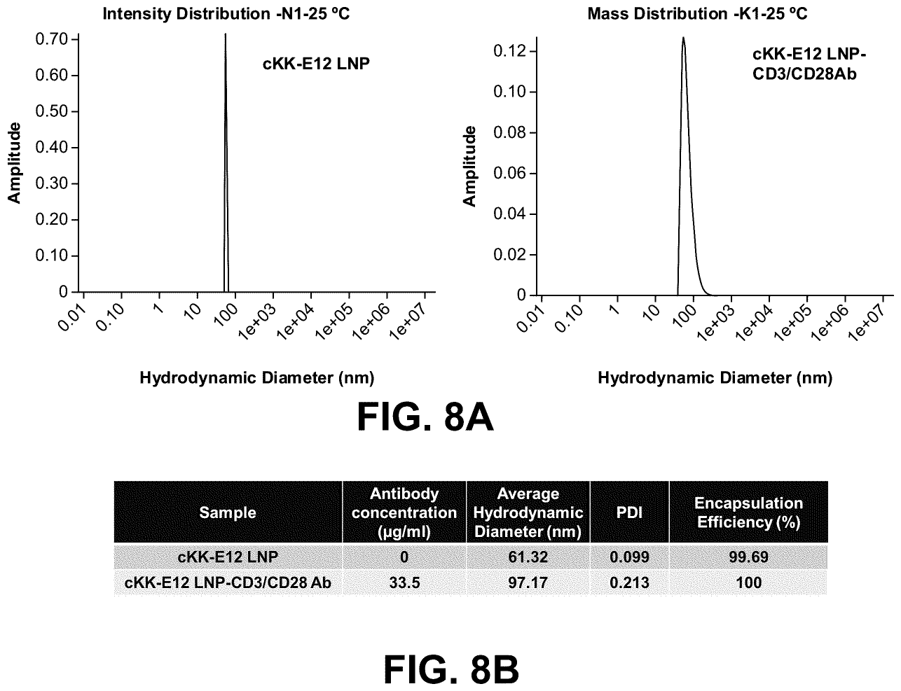

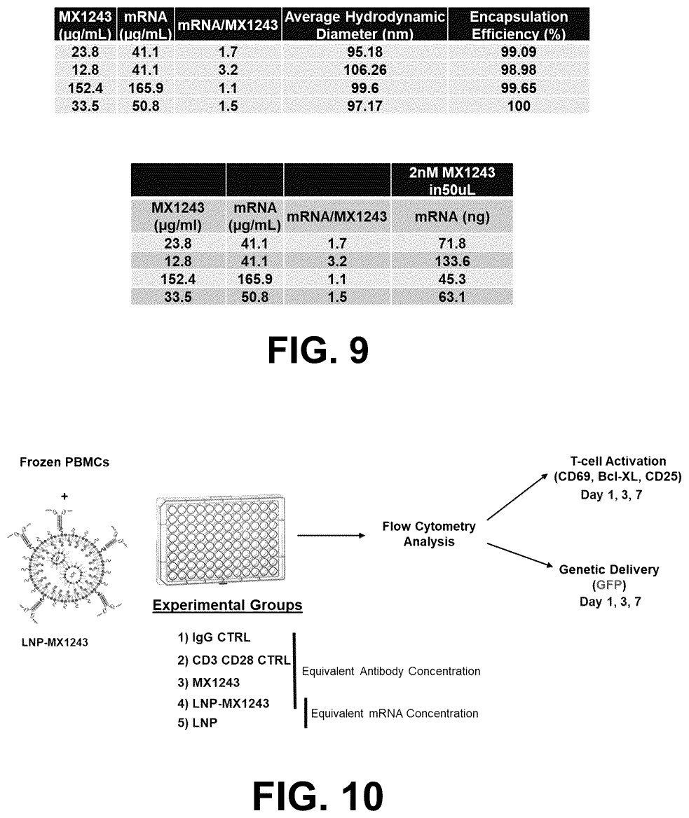

To measure the hydrodynamic diameter of LNPs, 10 μL of the LNP was added into UNcle (Unchained Labs) for a DLS assay with 10 measurements per sample (FIG. 8A). Average hydrodynamic diameter of unconjugated LNP was 61.32 nm while the average hydrodynamic diameter of CD3/CD28 (MX1243) antibody-conjugated LNP was 97.17 nm (FIG. 8B). Hydrodynamic diameter and encapsulation efficiency were consistent between batches of antibody-conjugated LNPs (FIG. 9).

mRNA content was measured using a RiboGreen assay (ThermoFisher). Conjugated or unconjugated LNPs were diluted 100-fold and mRNA standard (component C of the RiboGreen kit, corresponding to ribosomal 16S and 23S rRNA from E. coli at 100 μg/mL in TE buffer) was added at a concentration of 2 μg/mL in PBS. Six-fold dilution down to 31.25 ng/mL was performed in a 96 well plate. Ribogreen dye with and without 10% Triton X-100 (non-ionic surfactant) in PBS was added to the plate. Plates were put on a shaker for 15 minutes at 37° C. and measured using a plate reader at 480 nm excitation and 520 nm emission. The encapsulation efficiency was calculated as 1-(RNA measurement without Triton X-100/RNA measurement with Triton X-100). The nucleic acid encapsulation efficiency was consistently high in various LNP-MX1243 batches (FIG. 8B and FIG. 9). FIG. 9 shows different mRNA payloads (41.1 to 165.9 mg of mRNA per mL) and targeting antibody MX1243 (12.8 to 152.4 mg per mL of anti-CD3/anti-CD28 targeting antibody) were used, corresponding to mRNA/MX1243 ratios between 1.1 and 3.2. Across these ranges of payloads and targeting molecules, encapsulation efficacy was close to 100%, and the size of the LNP was approximately 100 nm (99.55+/−4.82).

### Example 4

### In Vitro Human T Cell mRNA Delivery and Activation Assay

Human IGG1_LALA_PA (Cambridge biologics, Cat. No. 07-01-1018) and anti-CD3 (Biolegend, Cat. No. 317326) antibodies were resuspended in 1×PBS (Gibco, Cat. No. 10010-023) at concentrations of 40, 8, 1.6, 0.32, 0.064, 0.0128 nM to form IgG control (‘IgG CTRL’) and CD3 CD28 control (‘CD3 CD28 CTRL’). Fifty μL of each standard concentration were added to each well of a 96-well, flat bottom cell culture plate in eight replicates of each group at the six concentrations for both controls. Plates were incubated for 2 hours at 37° C. with 5% CO₂. After incubation, 50 μL of 1×PBS was gently removed and 50 μL of prewarmed RPMI (Thermofisher, Cat. No. 72400047) supplemented with 10% FBS (Gibco, Cat. No. S124150H, Lot. M20189) and Penicillin-Streptomycin (Thermofisher, Cat. No. 15140122) was added to the ‘IgG CTRL’ or ‘CD3 CD28 CTRL’ groups. For the ‘CD3 CD28 CTRL’ group, an anti-CD28 antibody (Biolegend, Cat. No. B30630) was added to each well for a final concentration of 5 μg/mL. The mRNA payload (GFP; SEQ ID NO: 534) concentration of LNP-MX1243 and LNP was determined by using Ribogreen Assay kit according to manufacturer's protocol. The GFP mRNA (SEQ ID NO: 534) comprises a 5′ UTR of SEQ ID NO: 535, a 3′ UTR of SEQ ID NO: 536, and ORF of SEQ ID NO: 537, and a poly A tail of SEQ ID NO: 538.

Conjugated antibody concentration for LNP-MX1243 was measured using ELISA as described in above protocol. The concentration of the purified antibodies such as MX1243 were determined by measure OD280 and divide by Extinction Coefficient. MX1243 was diluted in media to concentrations of 40, 8, 1.6, 0.32, 0.064, and 0.0128 nM. LNP-MX1243 was diluted to a 40 nM antibody concentration and serially diluted to 8, 1.6, 0.32, 0.064, and 0.0128 nM in media. A non-conjugated LNP (‘LNP’), at an mRNA concentration equivalent to the mRNA concentration of LNP-MX1243, was resuspended in media and serially diluted similar to LNP-MX1243. Fifty μL of the MX1243, LNP-MX1243, and LNP samples were added to each well (FIG. 10).

During plate incubation, frozen PBMCs from 2 donors (Donor 1—Research Blood Components, Cat. No. (002), KP63473; Donor 2—Research Blood Components, Cat. No. (002), KP63574) were thawed in pre-warmed media. Cells were thawed in 500 μL of pre-warmed media and transferred to a 15 mL Falcon tube containing 9.5 mL of pre-warmed media. Cell suspensions were gently mixed and centrifuged at 250×g for 8 minutes. Cells were counted (Invitrogen, Countess 3 FL) and rested for 1 hour at 37° C., 5% CO₂ in media. Cultured cells were then re-suspended and strained using a 70 μm cell strainer (Falcon, Cat. No. 352350). Cells were then pelleted at 350×g for 5 minutes, counted (Invitrogen, Countess 3 FL) and resuspended to 3.3×105 cells/mL in pre-warmed medium. 150 μL were added to each well (50,000 PBMC). Cells were incubated at 37° C., 5% CO₂ for 24 hours, 72 hours, and 168 hours. Delivery of mRNA was assessed by GFP expression while T cell activation was assessed by CD69, Bcl-XL, and CD25 expression (FIG. 10).

### Example 5

### Cell Staining for Flow Cytometry Analysis

At indicated time points (24, 72, and 168 hours), cells were transferred to U-bottom, 96 well plates. Plates were centrifuged at 350×g for 5 minutes and supernatants were removed. Cells were resuspended in 200 μL cold 1×PBS, centrifuged at 350×g for 5 minutes, and supernatants were removed. Viability staining dye (Invitrogen, eFluor 506, Cat. No. 65086618) was diluted 1:1000 in 1×PBS and cell pellets were resuspended in 50 μL of the solution. While covered from light, cell suspensions were incubated at 4° C. for 10 minutes.

During incubation, a master mix of staining antibodies (CD69-PerCP-Cy5.5, Biolegend, Cat. No. 310926; CD2-APC/C7, Biolegend, Cat. No. 300220; CD25-BV421, Biolegend, Cat. No. 302630; CD4-BV605, Biolegend, Cat. No. 317438; CD8-PE-CY7, Fisher, Cat. No. 50-154-49) and isotype controls (PerCP-Cy5.5, Biolegend, Cat. No. 400149; BV421, Biolegend, Cat. No. 400157) was prepared in FACS buffer (1×PBS, 1% Bovine Serum Albumin (Fisher, Cat. No. BP1600-1), 0.1% Sodium Azide (Ricca, Cat. No. 7144.8-16)).

Fifty μL of the isotype control stain or master mix were added to each well and cell suspensions were gently mixed. While covered from light, cell suspensions were incubated at 4° C. for 20 minutes. Cell pellets were generated by centrifugation at 350×g for 5 minutes and cell pellets were washed in 200 μL of cold FACS buffer. This step was repeated. Cell pellets were then resuspended in 50 μL of BD Cytofix/Cytoperm Solution (BD, Cat. No. 554722). While covered from light, cell suspensions were incubated at 4° C. for 20 minutes.

Cell pellets were generated by centrifugation at 500×g for 5 minutes and cell pellets were washed in 200 μL of cold BD Perm/Wash Solution (BD, Cat. No. 554723) buffer. This step was repeated. A solution of staining antibody (Bcl-xl-PE, Southern Biotech, Cat. No. 10030-09) or isotype control (PE, Southern Biotech, Cat. No. 0105-09) was prepared in cold BD Perm/Wash Solution (BD, Cat. No. 554723) buffer. Fifty μL of either the isotype control or staining antibody solution were used to resuspend the cell pellets.

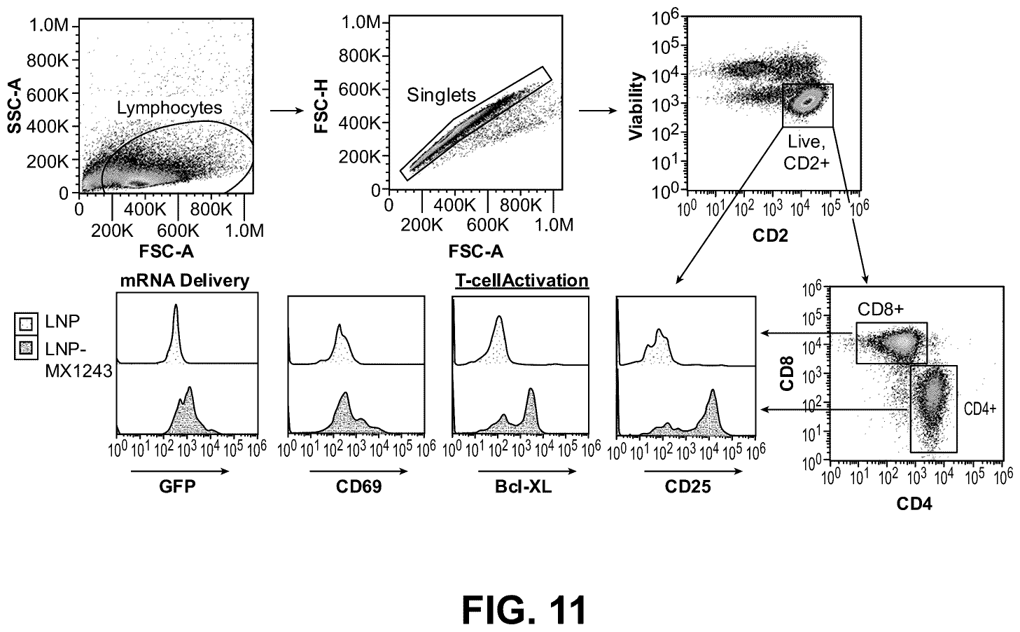

While covered from light, cell suspensions were incubated over-night at 4° C. Cell pellets were generated by centrifugation at 500×g for 5 minutes and cell pellets were washed in 200 μL of cold BD Perm/Wash Solution (BD, Cat. No. 554723) buffer. This step was repeated. Cell pellets were then resuspended in 150 μL of cold BD Perm/Wash Solution (BD, Cat. No. 554723). One hundred fifteen μL were used to acquire fluorescence on an Attune flow cytometer. Gating was performed to evaluate cellular populations with the size and granular characteristics of lymphocytes. Singlet, viable T cells (Live, CD2⁺) were analyzed for GFP, CD69, Bcl-XL, and CD25. Total viable T cell populations (Live, CD2⁺) were subdivided into CD8+ and CD4+ T cell subsets. These T cell subsets were then analyzed for GFP, CD69, Bcl-XL, and CD25 (FIG. 11). FIG. 11 shows that gating strategy for identifying different populations of the cells.

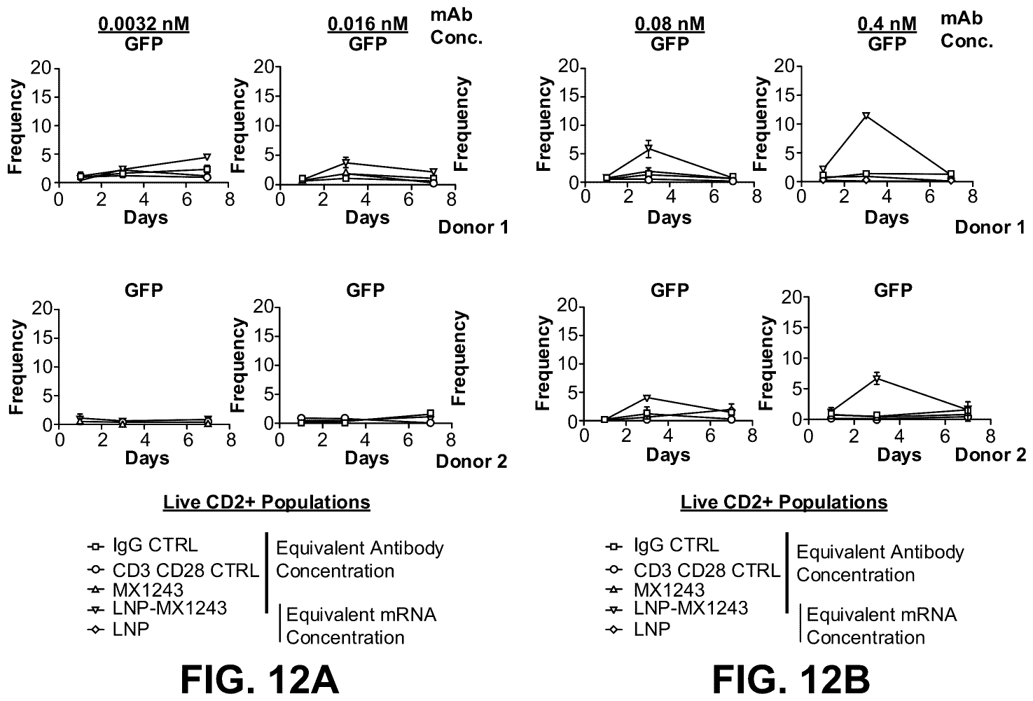

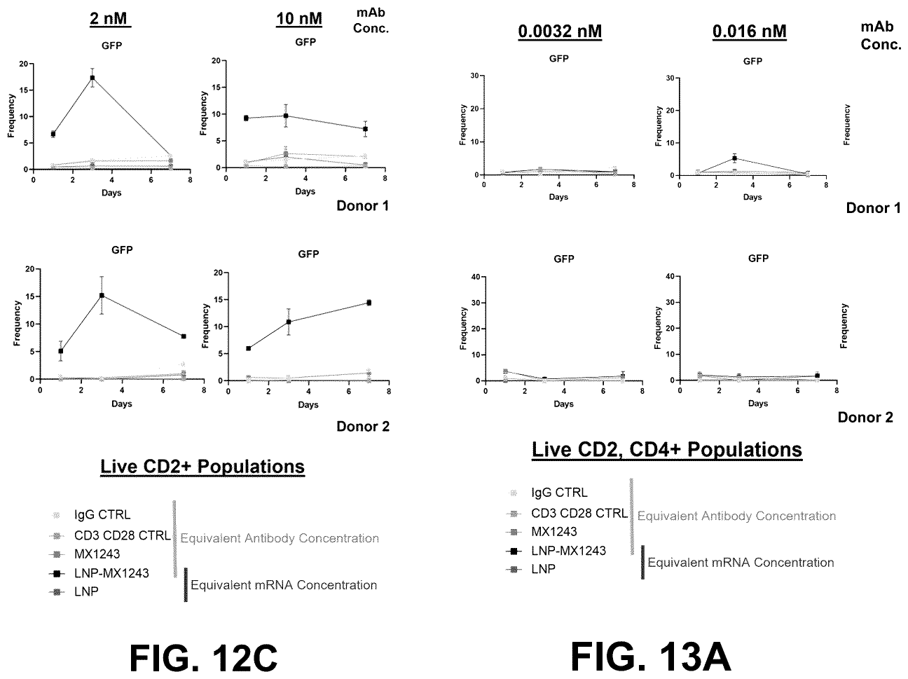

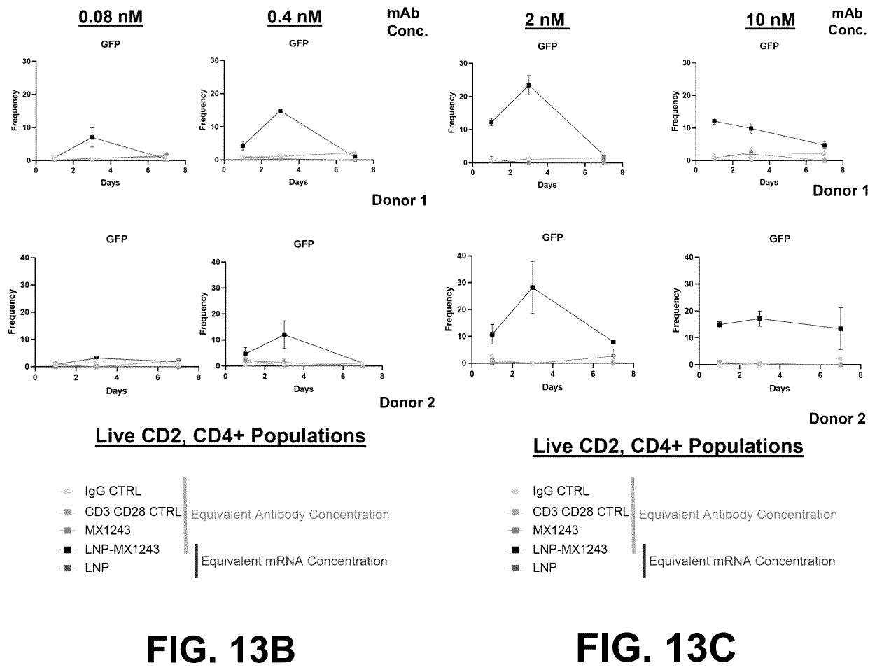

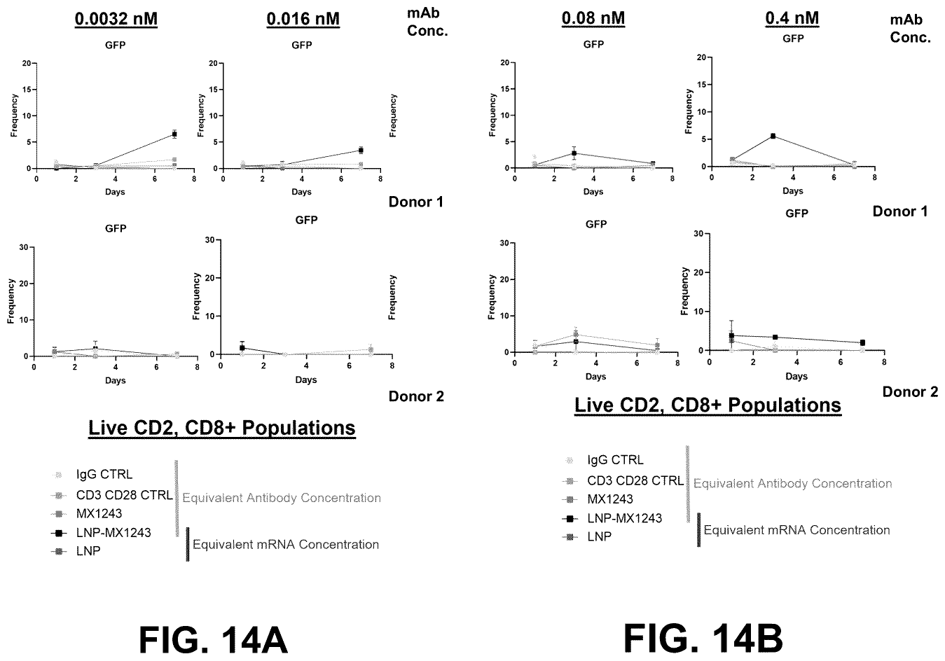

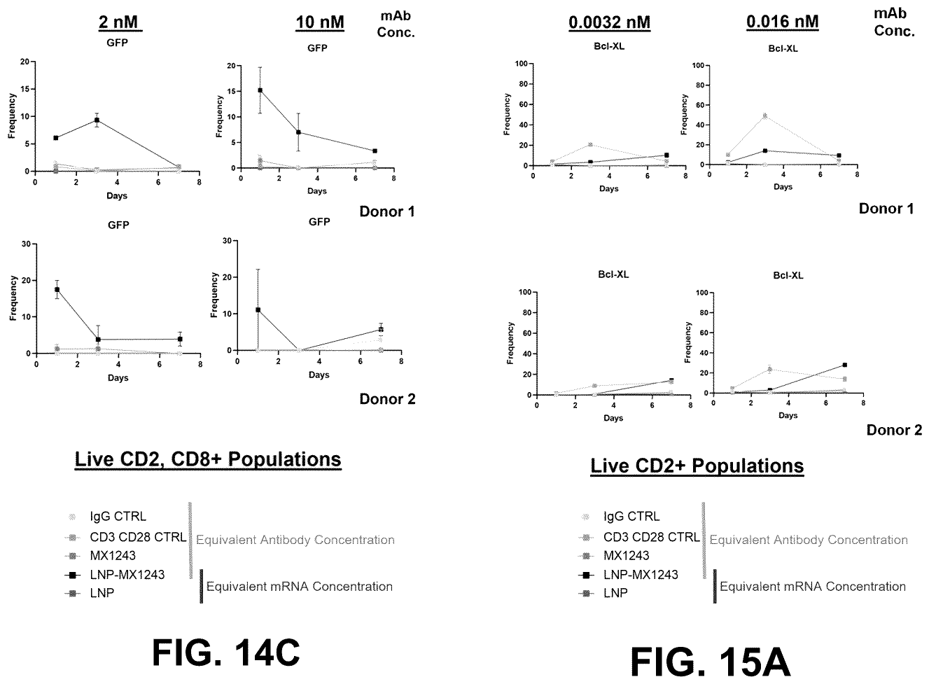

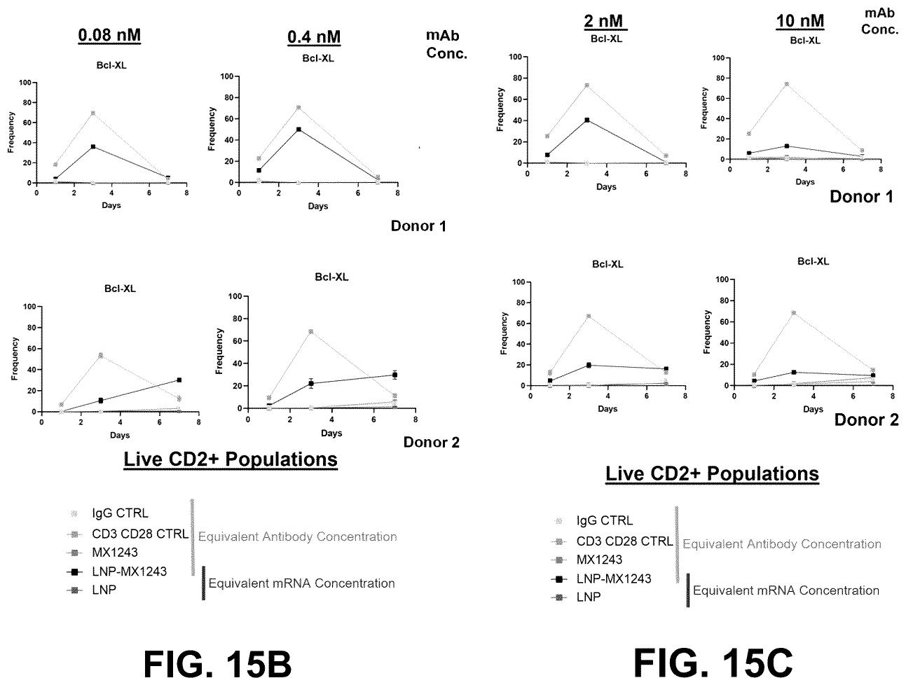

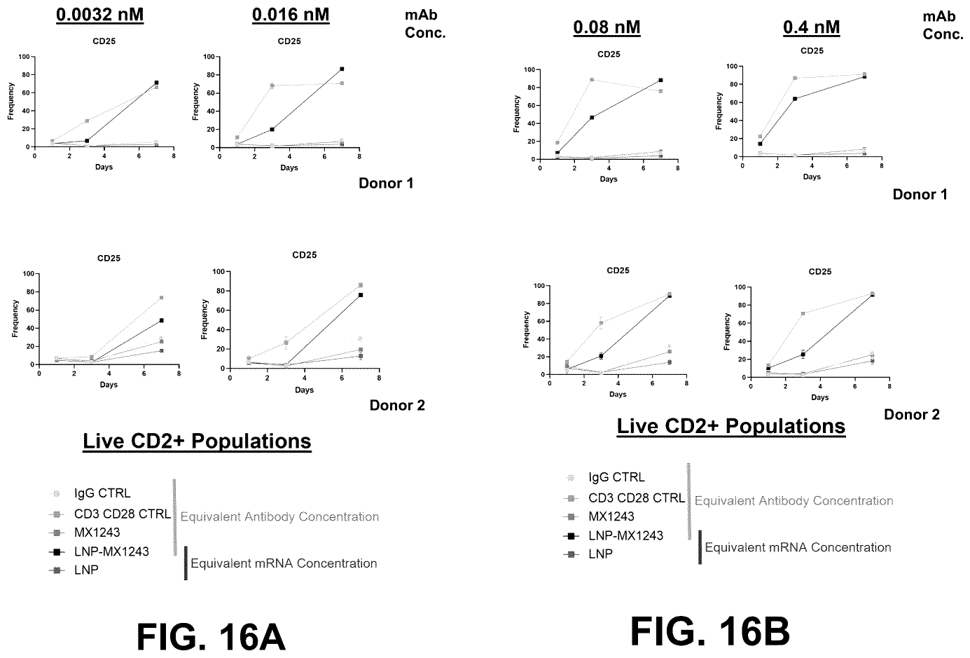

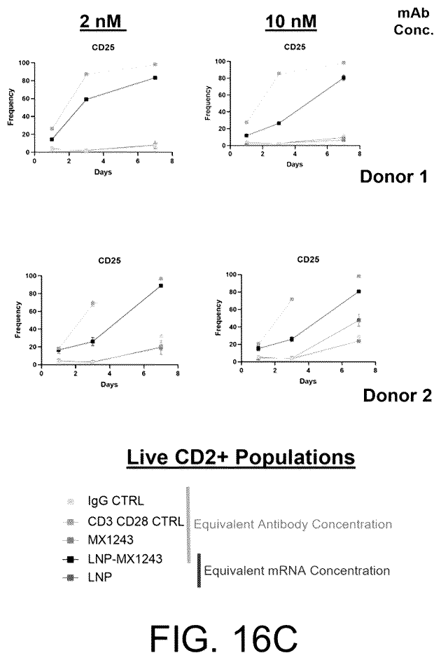

Time course analysis (1, 3, and 7 days post-treatment) of GFP+ cells within viable total T cell populations (Live, CD2⁺) of PBMC from 2 healthy donors demonstrated that LNP-MX1243 specifically delivered GFP mRNA to T cells relative to unconjugated control (FIGS. 12A, 12B, 12C). Time course analysis (1, 3, and 7 days post-treatment) of the frequency of GFP+ cells within CD4+ T cell populations (Live, CD2+, CD4+) of PBMC from 2 healthy donors demonstrated that LNP-MX1243 delivered mRNA to CD4+ T cells relative to unconjugated control (FIGS. 13A, 13B, 13C). Time course analysis (1, 3, and 7 days post-treatment) of the frequency of GFP+ cells within CD8+ T cell populations (Live, CD2+, CD8+) of PBMC from 2 healthy donors demonstrated that LNP-MX1243 delivered mRNA to CD8+ T cells relative to unconjugated control (FIGS. 14A, 14B, 14C). Furthermore, time course analysis (1, 3, and 7 days post-treatment) of the frequency of Bcl-XL+ cells within viable total T cell populations (Live, CD2⁺) of PBMC from 2 healthy donors demonstrated that LMP-MX1243 activated T cells at similar rates to a CD3 CD28 antibody control (FIGS. 15A, 15B, 15C). Time course analysis (1, 3, and 7 days post-treatment) of the frequency of CD25+ cells within viable total T cell populations (Live, CD2⁺) of PBMC from 2 healthy donors demonstrated that LMP-MX1243 activated T cells at similar rates to the CD3 CD28 antibody control (FIGS. 16A, 16B, 16C).

### Example 6

### Generation of NSG Mice with Reconstituted Human PBMCs

Frozen PBMCs from a human donor (Research Blood Components, Cat. No. (002), KP66224) were thawed in pre-warmed media. Cells were thawed in 500 μL of pre-warmed media and transferred to a 15 mL Falcon tube containing 9.5 mL of pre-warmed media. Cell suspensions were gently mixed and centrifuged at 250×g for 8 minutes. Cells were counted (Invitrogen, Countess 3 FL) and rested for 1 hour at 37° C., 5% CO₂ in media.

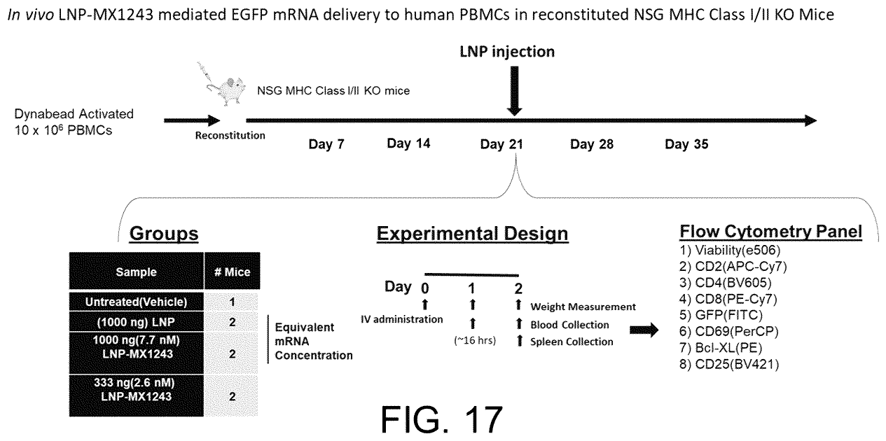

Cultured cells were re-suspended and strained using a 70 μm cell strainer (Falcon, Cat. No. 352350). Cells were then pelleted at 350×g for 5 minutes, counted (Invitrogen, Countess 3 FL), and resuspended to 2×10⁶/mL in 6-well dishes. Cell cultures were stimulation with Dynabeads™ Human T-Activator CD3/CD28 for T Cell Expansion and Activation (Thermofisher, Cat. No. 11161D) at 1:10 Bead/PBMC ratio and incubated at 37° C., 5% CO₂ for 2 days (FIG. 17).

Cultures were transferred to 15 mL Falcon tubes and Dynabeads were removed through use of a cell culture magnet (Invitrogen, DYNAL). Cell suspensions were strained using a 70 μm cell strainer (Falcon, Cat. No. 352350). Cell pellets were generated by centrifugation at 350×g for 5 minutes and cell pellets were resuspended in 10 mL 1×PBS. Cells were counted (Invitrogen, Countess 3 FL) and resuspended to 25×10⁶/mL in 1×PBS. For reconstitution, 10×10⁶ cells were injected IP into each mouse. The reconstitution status was monitored by analyzing human T cell numbers in mouse blood weekly. Generally 10-40% of human CD2+ T cells were observed in each mouse between 3-5 weeks post reconstitution.

### Example 7

### Splenocyte Isolation

Spleens were suspended in 10 mL of media in 6 well dishes and cut into small pieces with dissection forceps/scissors. Tissue pieces were mashed with a 25 mL syringe and filtered using 70 μm cell strainers (Falcon, Cat. No. 352350). Splenocyte suspensions were transferred to a 50 mL Falcon tube and filtered again using 70 μm cell strainers (Falcon, Cat. No. 352350). Cell pellets were generated through centrifugation at 400×g for 5 minutes. Cell pellets were resuspended in 4 mL of ACK Lysis Buffer (Gibco, Cat. No. A1049201) and pelleted through centrifugation at 400×g for 5 minutes.

Cell pellets were resuspended in 10 mL of media and filtered using 70 μm cell strainers (Falcon, Cat. No. 352350). Cell pellets were generated by centrifugation at 400×g for 5 minutes. This step was repeated. Cell pellets were resuspended in 4 mL of FACS buffer (PBS buffer with 1% Bovine Serum Albumin (BSA) (Sigma, Catalog No. A0336-50 ML) and 0.1% Sodium Azide (RICCA, Catalog No. 7144.8-16)), and counted (Invitrogen, Countess 3 FL). Two hundred thousand cells per sample were subjected to staining for flow cytometry analysis. See Example 5.

### Example 8

### Blood Processing and Cell Staining for Flow Cytometry Analysis

Whole blood was transferred to 1.5 mL Eppendorf tubes. Blood was then resuspended in 500 μL of 1×PBS and pelleted by centrifugation at 500×g for 5 minutes. This step was repeated. Cell pellets were then resuspended in 100 μL diluted Viability dye (1/1000 stock in 1×PBS, Invitrogen, eFluor 506, Cat. No. 65086618) and incubated for 30 minutes covered from light at 4° C. During incubation, a master mix of staining antibodies (CD69-PerCP-Cy5.5, Biolegend, Cat. No. 310926; CD2-APC/C7, Biolegend, Cat. No. 300220; CD25-BV421, Biolegend, Cat. No. 302630; CD4-BV605, Biolegend, Cat. No. 317438; CD8-PE-CY7, Fisher, Cat. No. 50-154-49) and isotype controls (PerCP-Cy5.5, Biolegend, Cat. No. 400149; BV421, Biolegend, Cat. No. 400157) was prepared in FACS buffer (1×PBS, 1% Bovine Serum Albumin (Fisher, Cat. No. BP1600-1), 0.1% Sodium Azide (Ricca, Cat. No. 7144.8-16)).

One hundred μL of the isotype control stain or master mix were added to each sample and cell suspensions were gently mixed. While covered from light, cell suspensions were incubated at 4° C. for 60 minutes. Samples were then washed with 500 μL of FACS buffer and pelleted by centrifugation at 500×g for 5 minutes. This step was repeated. Cell pellets were gently resuspended in 1.0 mL of ACK Lysis Buffer (Gibco, Cat. No. A1049201) and pelleted by centrifugation at 500×g for 5 minutes. Cell pellets were washed by gentle resuspension in 500 μL of FACS buffer and pelleted by centrifugation at 500×g for 5 minutes. This step was repeated.

Cell pellets were then resuspended in 100 μL of BD Cytofix/Cytoperm (BD, Cat. No. 51-2090KZ) and transferred to 96-well U bottom plates. While covered from light, cell suspensions were incubated at 4° C. for 20 minutes. Cell pellets were generated by centrifugation at 700×g for 5 minutes. Cell pellets were resuspended in 200 μL of BD Perm/Wash (BD, Cat. No. 51-2091KZ) and pelleted by centrifugation at 700×g for 5 minutes. This step was repeated.

A solution of staining antibody (Bcl-xl-PE, Southern Biotech, Cat. No. 10030-09) or isotype control (PE, Southern Biotech, Cat. No. 0105-09) was prepared in cold BD Perm/Wash Solution (BD, 554723) buffer. One hundred μL of either the isotype control or staining antibody solution was used to resuspend the cell pellets. While covered from light, cell suspensions were incubated over-night at 4° C. Cell pellets were generated by centrifugation at 700×g for 5 minutes and were washed by resuspension in 200 μL of BD Perm/Wash (BD, Cat. No. 51-2091KZ). This step was repeated. Cell pellets were then resuspended in 150 μL BD Perm/Wash (Cat. No. 51-2091KZ) and 25 μL of Precision Counting Beads (Biolegend, Cat. No. 424902) were added to each sample. Acquisition was performed on an Attune flow cytometer. One hundred fifty μL of each sample was acquired.

### Example 9

### In Vivo Delivery of Conjugated LNPs to Circulating Human Primary T Cells in NSG Mice Reconstituted with Human PBMCs

NSG MHC Class I/II KO mice (Jackson, Strain #:025216) were infused with 10×10⁶ Dynabead-activated human PBMCs (FIG. 17). Sufficient human immune cell reconstitution occurred at approximately 3 weeks post-infusion. Mice were treated on day 21 post-infusion intravenously with 1000 ng LNP, 1000 ng (7.7 nM) LNP-MX1243, or 333 ng (2.6 nM) LNP-MX1243, respectively. An equivalent amount of encapsulated mRNA was used for the LNP-MX1243 and LNP 1000 ng groups. Whole blood was analyzed for mRNA delivery and T cell activation at 16 and 36 hours post-treatment. The splenic compartment was analyzed for mRNA delivery and T cell activation at 36 hours post-treatment (FIG. 17).

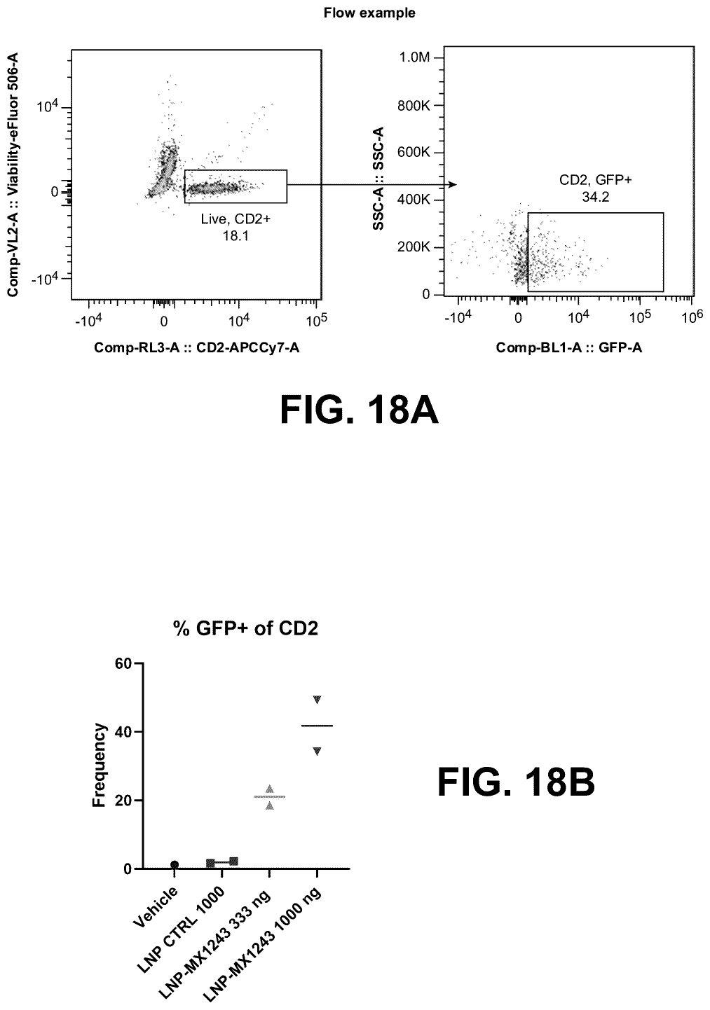

A scatter plot indicating gating for the evaluation of GFP+ cells populations within viable T cell populations (Live, CD2⁺) is shown in FIG. 18A. The frequency of GFP+ cells within viable T cell populations (Live, CD2⁺) in the blood at 16 hours post-treatment increased with increasing concentrations of LNP-MX1243 (FIG. 18B). Notably, MX1243 increased the frequency of GFP+ cells in a dose dependent manner by ˜20 fold (FIG. 18B).

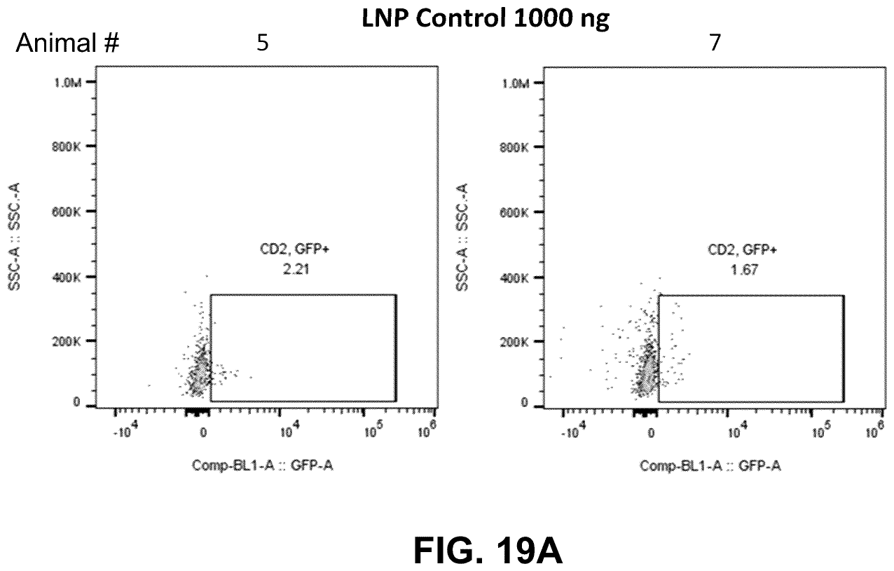

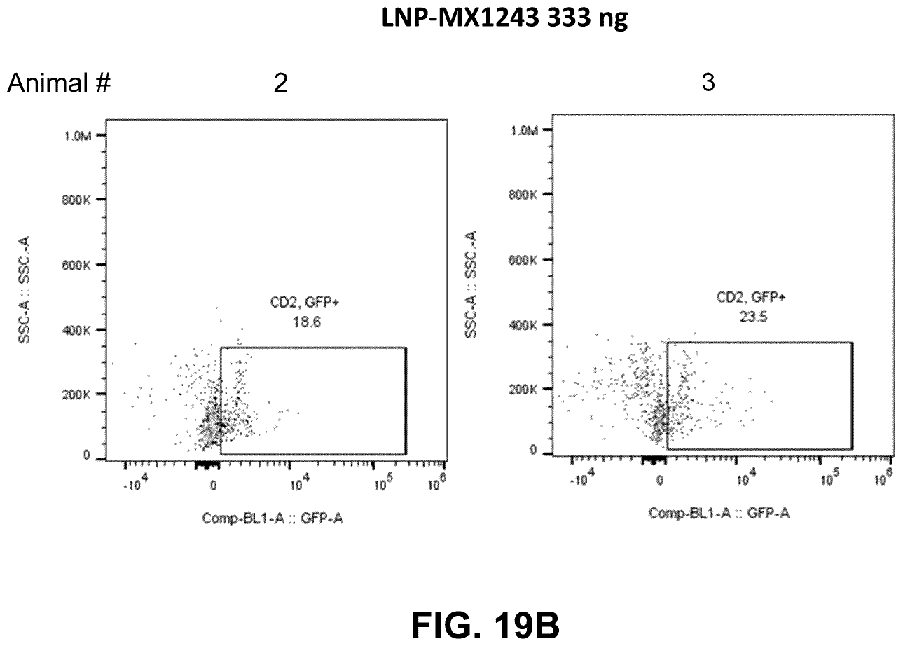

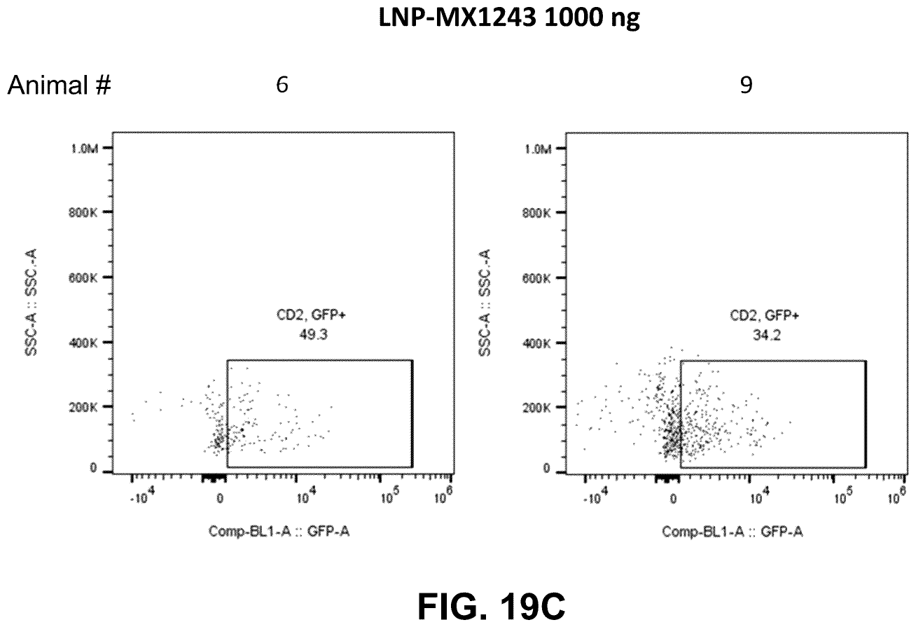

Scatter plots of individual animals demonstrated the frequency of GFP+ cells within viable T cell populations (Live, CD2⁺) in the blood at 16 hours post-treatment with 1000 ng LNP controls (FIG. 19A), 333 ng LNP-MX1243 (FIG. 19B), and 1000 ng of LNP-MX1243 (FIG. 19C).

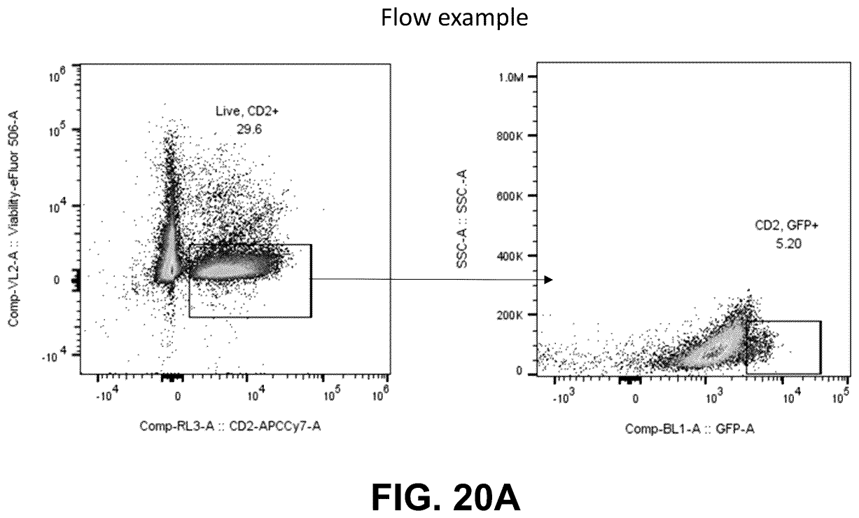

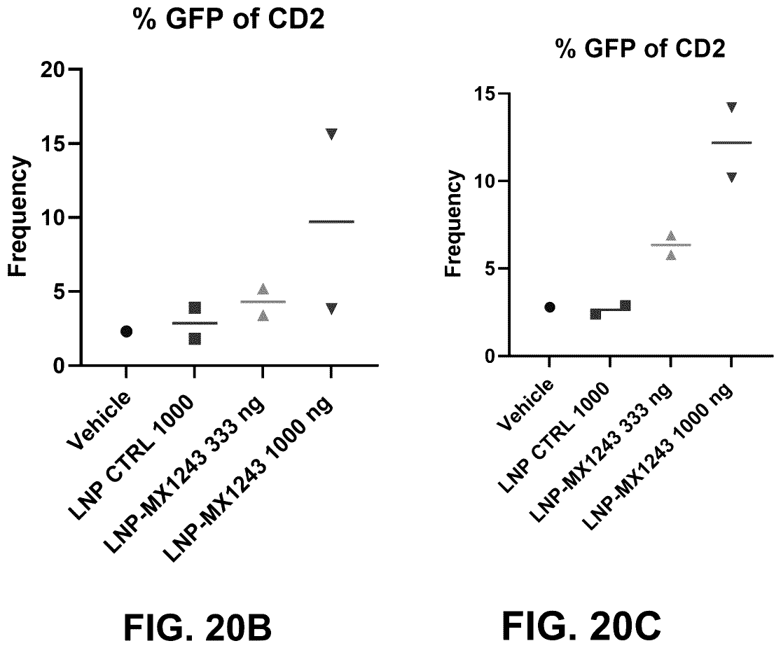

Scatter plots of splenocytes representing GFP+ cells within viable, total T cell populations (Live, CD2⁺) are shown in FIG. 20A. The frequency of GFP+ cells within viable T cell populations (Live, CD2⁺) in the blood at 36 hours post-treatment increased with increasing concentrations of LNP-MX1243 (FIG. 20B). Similarly, the frequency of GFP+ cells within viable T cell populations (Live, CD2⁺) in the spleen at 36 hours post-treatment increased with increasing concentrations of LNP-MX1243 (FIG. 20C).

Taken together, these results demonstrated that maleimide-thiol reaction-based conjugation efficiently attached antibody molecules to the LNP surface. LNP-MX1243 effectively activated human primary T cells while also delivering mRNA cargo into T cells resulting in transgene expression. In a human PBMC reconstituted NSG MHC Class I/II KO mouse model, LNP-MX1243 effectively delivered mRNA cargo into circulating human T cells resulting in EGFP expression in a high percentage of human T cells. Overall, these results demonstrated successful delivery of GFP mRNA to human T cells both in vitro and in vivo using CD28/CD3 antibody-conjugated LNPs.

### Example 10

### Bivalency and Optimization of Antibody Ratios

For successful delivery of LNP cargo to T cells at least two important steps are required. The first step is specific delivery to T cells which can be achieved by anti-CD3, or anti-CD28, or both anti-CD3 and antiCD28. This step includes LNP attachment to T cell and subsequent LNP internalization through endocytosis. The second required steps in T cell activation. This step is required for gene expression post-delivery and T cell survival to perform T cell therapeutic functions. This matrix study was designed to investigate various ways of conjugating monoclonal antibodies to LNP for optimal T cell delivery and T cell functions.

To investigate the contribution of two targeting antibodies on T cell targeting and activation, LNPs with two antibody species conjugated to their surface and mixtures of LNPs with single antibody species conjugated to their surface were generated and tested. Antibodies used were either monospecific (MX1506 (key chain SEQ ID NO: 7; hole chain SEQ ID NO: 25; light chain SEQ ID NO: 39) and MX1507 (key chain SEQ ID NO: 5; hole chain SEQ ID NO: 23; light chain SEQ ID NO: 37) or bispecific (MX1243 (key chain SEQ ID NO: 18; hole chain SEQ ID NO: 36) and MX1500 (key chain SEQ ID NO: 8; hole chain SEQ ID NO: 26) (FIG. 21). In the first approach, two monospecific antibodies (MX1506 and MX1507) were mixed prior to LNP generation to prepare bivalent LNPs (FIG. 22). In the second approach, single monospecific antibodies were conjugated separately to LNPs and the single-antibody LNPs mixed to prepare mixtures of two monovalent LNPs (FIG. 23). TABLE 18 shows the different LNPs prepared. All LNPs were formulated with cKK-E12 (3,6-bis[4-[bis(2-hydroxydodecyl)amino]butyl]-2,5-piperazinedione; CAS number 1432494-65-9).

TABLE 18
Ratios of antibodies attached to the surface of LNPs.

| | MX1243 | MX1506 | MX1507 | MX1500 |
|---|---|---|---|---|
| | Bispecific | Monospecific | Monospecific | Bispecific |
| ID | CD3/CD28 | CD28 | CD3 | CD3/CD28 |
| LNP-1 | 0% | 0% | 0% | 0% |
| LNP-2 | 0% | 100% | 0% | 0% |
| LNP-3 | 0% | 75% | 25% | 0% |
| LNP-4 | 0% | 50% | 50% | 0% |
| LNP-5 | 0% | 25% | 75% | 0% |
| LNP-6 | 0% | 0% | 100% | 0% |
| LNP-7 | 0% | 75% | 25% | 0% |
| LNP-8 | 0% | 50% | 50% | 0% |
| LNP-9 | 0% | 25% | 75% | 0% |
| LNP-10 | 100% | 0% | 0% | 0% |
| LNP-11 | 0% | 0% | 0% | 100% |

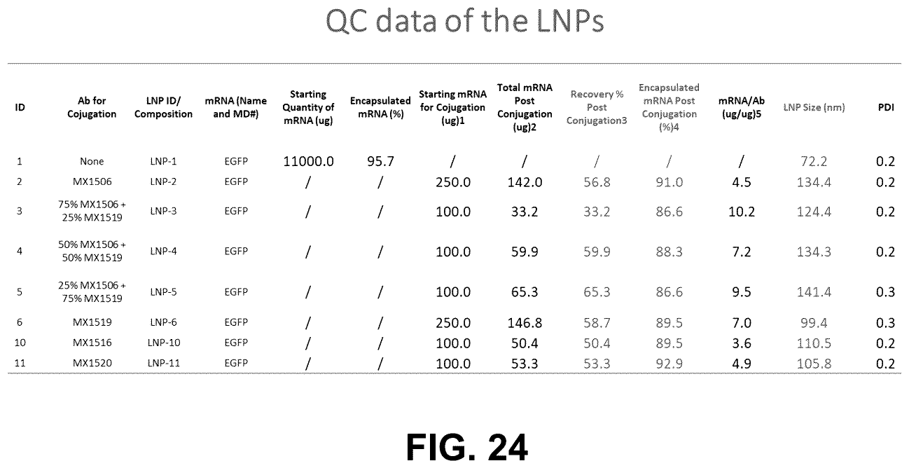

Quality control data for the LNPs used in this experiment is presented in FIG. 24. Size exclusion chromatography (SEC) of LNPs conjugated with a single antibody species (LNP-1, -2 and -6) demonstrated efficient conjugation with antibody MX1506. Antibodies MX1243 and MX1507 showed less efficient conjugation. For example, only about 1.5% of MX1507 were recovered indicating that 98.5% of MX1507 were lost potentially due to precipitation/aggregation with this antibody.

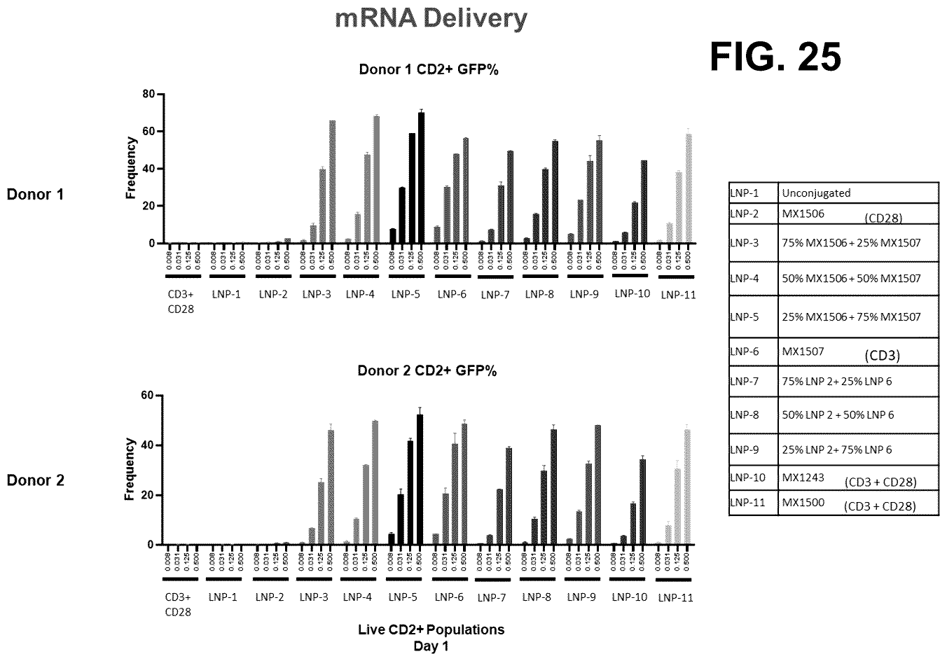

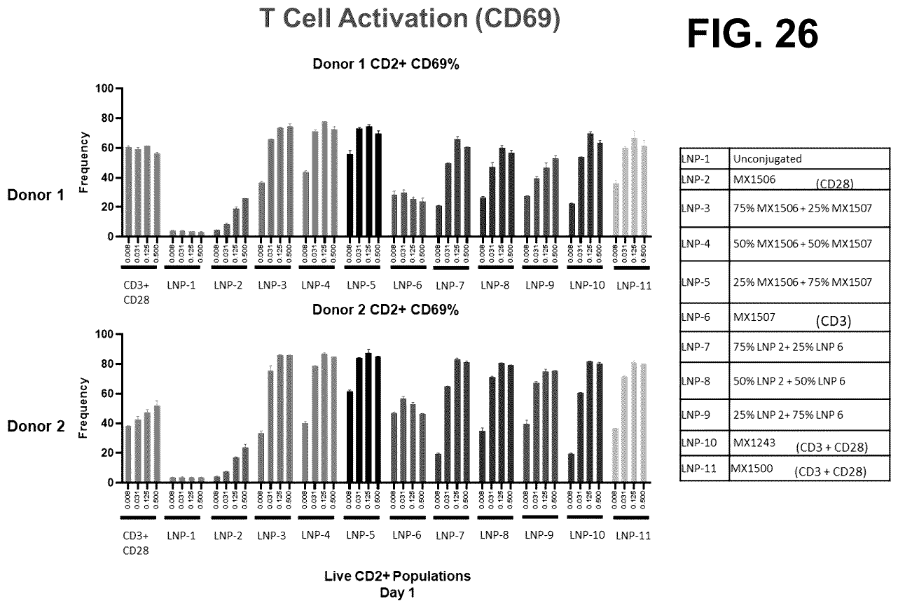

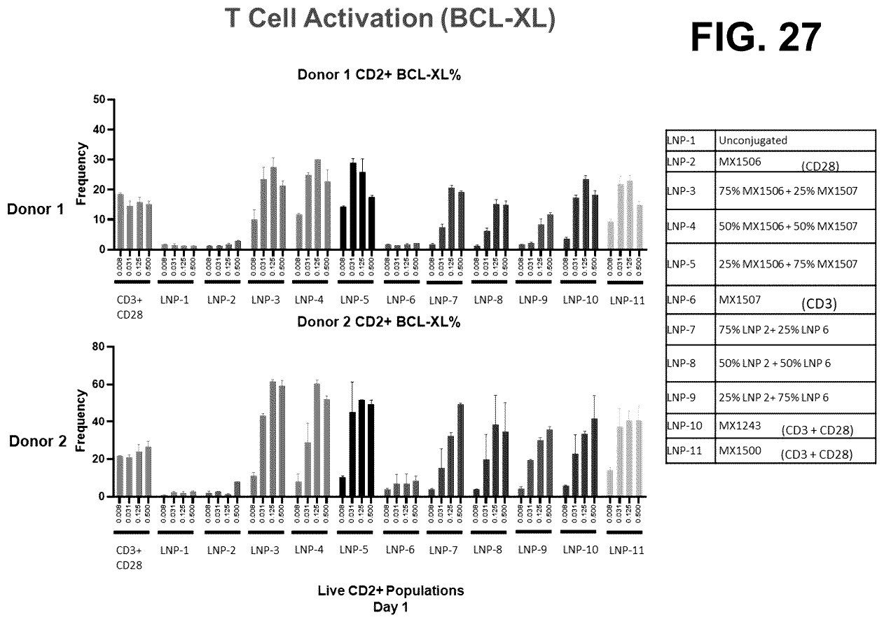

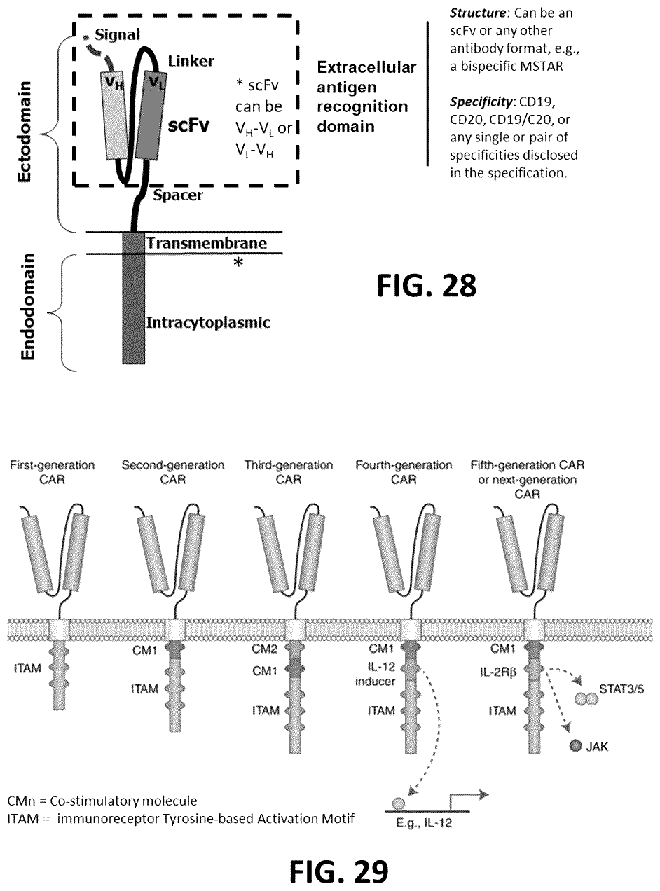

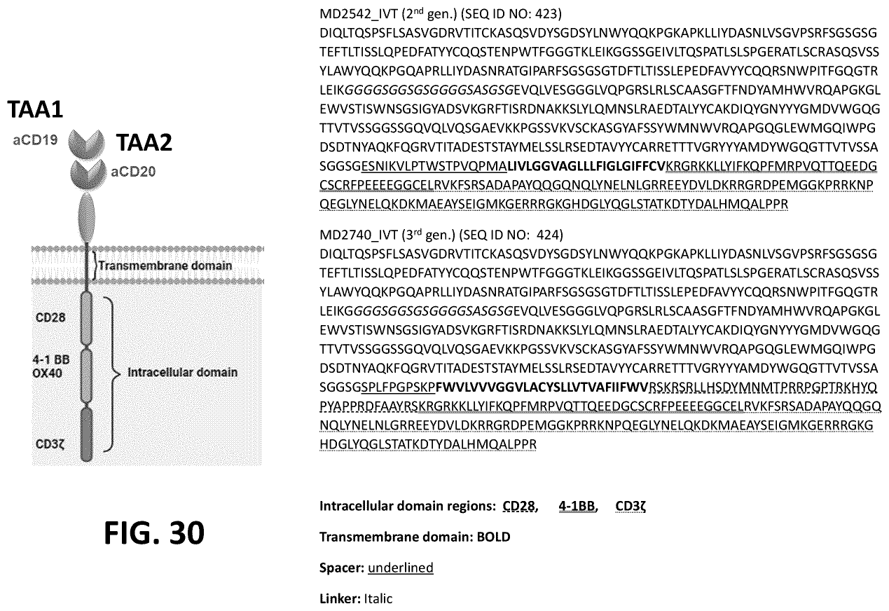

GFP mRNA delivery to T cells in vitro was tested to assess cargo delivery. Concentration-dependent (μg mRNA/ml) increases in GFP+ viable T cells were observed in PBMC of two different donors after treatment with LNP-1 to LNP-11 (FIG. 25). T cell activation in vitro was also tested. Activation was quantitated by measuring the expression levels of CD69 on T cells. Concentration-dependent (μg mRNA/ml) increases in of CD69 were observed in PBMC of two different donors after treatment with LNP-1 to LNP-11 (FIG. 26). Bcl-XL expression on T cells was used for assessing T cell survival. Concentration-dependent (μg mRNA/ml) increases in Bcl-XL+ viable T cells were also observed in PBMC of two different donors after treatment with LNP-1 to LNP-11 (FIG. 27). These results demonstrated that LNP conjugated to MX1507 (LNP-6) was sufficient to deliver cargo (GFP mRNA) but MX1506 (LNP-3, -4, -5) enhanced delivery efficiency. MX1507 was not sufficient for upregulating BCL-XL expression and MX1506 was required to upregulate BCL-XL, which is essential for T cell survival post activation. Bivalent LNP-3 and LNP-4 outperformed LNP-MX1243 (LNP-10) in mRNA delivery indicating bivalency may enhance cargo delivery. Furthermore, the LNPs that had the antibodies conjugated to the same particle (LNP-3, -4, and -5) performed significantly better than a mix of single conjugated LNPs (LNP-7, -8, and -9).

Conclusions: Targeting CD3 by itself was sufficient to deliver the payload to T cells, but targeting CD28 was also required to upregulate Bcl-XL. Targeting CD28 by itself was not sufficient to deliver cargo or upregulate Bcl-XL. LNP-3, LNP-4 and LNP-5 outperformed LNP-MX1243 in mRNA delivery. All LNPs except for LNP-2 and LNP-6 showed similar T cell activation in terms of both CD69 and Bcl-XL upregulation The LNPs that had the antibodies conjugated on the same particle (LNPs 3-5) performed better than a mix of single conjugated LNPs (LNPs 7-9) at least for mRNA delivery. LNP-MX1500 showed higher mRNA delivery than LNP-MX1243.

### Example 11

### Suitability of Various LNP Formulations

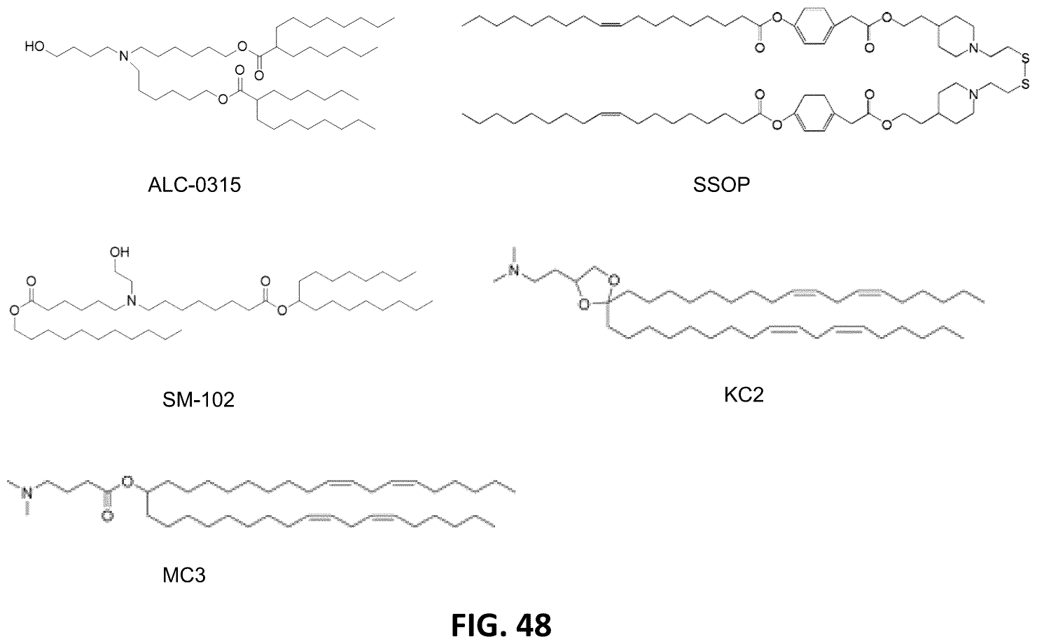

Several commercially available LNP formulations were compared to the cKK-E12 used in the above experiments. ALC-0315 (available, e.g., from Echelon Biosciences, Product Number: N-1020); SM-102 (available, e.g., from Echelon Biosciences, Product Number: N-1102); SSOP (available, e.g., from NOF America Corporation, COATSOME SS—OP): KC2 (term used interchangeably with DLin-KC2-DMA) (available from, e.g., Cayman Chemical Item No. 34363, CAS No. 1190197-97-7); and, MC3 (term used interchangeably with DLin-MC3-DMA) (available from, e.g., Cayman Chemical Item No. 34364, CAS No. 1224606-06-7) were used to prepare ALC-0315-LNPs, SM-102-LNPs, and SSOP-LNPs. See structure in FIG. 48.

LNP formulations were prepared using the same N/P ratio, the same mole ratio among lipids, the same mRNA content, the same maleimide-antibody ratio, and the same flow rate during formulation. Encapsulation efficiency (EE %) was calculated based on the concentration of total mRNA and encapsulated RNA determined by Ribogreen assay. Percentage of recovery of encapsulated mRNA (Recovery %) was calculated based on the total amount of encapsulated mRNA in LNP samples and total mRNA added during formulation. The EE % was similar between cKK-E12-LNPs, ALC-0315-LNPs, SM-102-LNPs, KC2, MC3 and SSOP-LNPs prior to MX1243 conjugation. The EE % was highest in cKK-E12-LNPs after MX1243 conjugation followed by SM-102-LNPs. The recovery % of encapsulated mRNA was highest in ALC-0315-LNPs prior to MX1243 conjugation. After MX1243 conjugation, the recovery % of encapsulated mRNA was highest in SM-102-LNPs followed by ALC-0315-LNPs (FIG. 26).

The hydrodynamic diameter of cKK-E12-LNPs, ALC-0315-LNPs, SM-102-LNPs, and SSOP-LNPs conjugated to MX1243 was determined by DLS. Except for ALC-0315-LNPs where small amounts of unconjugated ALC-0315 were observed, the remaining LNPs were efficiently conjugated. The antibody content was higher on ALC-0315-LNPs, SM-102-LNPs, and SSOP-LNPs compared to cKK-E12-LNPs. Similarly, conjugation efficiency/recovery % were higher with ALC-0315-LNPs, SM-102-LNPs, and SSOP-LNPs compared to cKK-E12LNPs. Notably, similar levels of T cell activation in PBMC of two different donors were observed for all MX1243 conjugated LNPs (cKK-E12-LNPs, ALC-0315-LNPs, SM-102-LNPs, and SSOP-LNPs), but only cKK-E12-LNPs showed effective mRNA delivery and EGFP expression. These results indicated that various LNP formulations could efficiently encapsulate mRNA, be conjugated to antibodies, and activate T cells; however, the mRNA delivery to T cells appeared to be most efficient with cKK-E12-LNPs.

### Example 12

### Selection of Ionizable Cationic Lipid

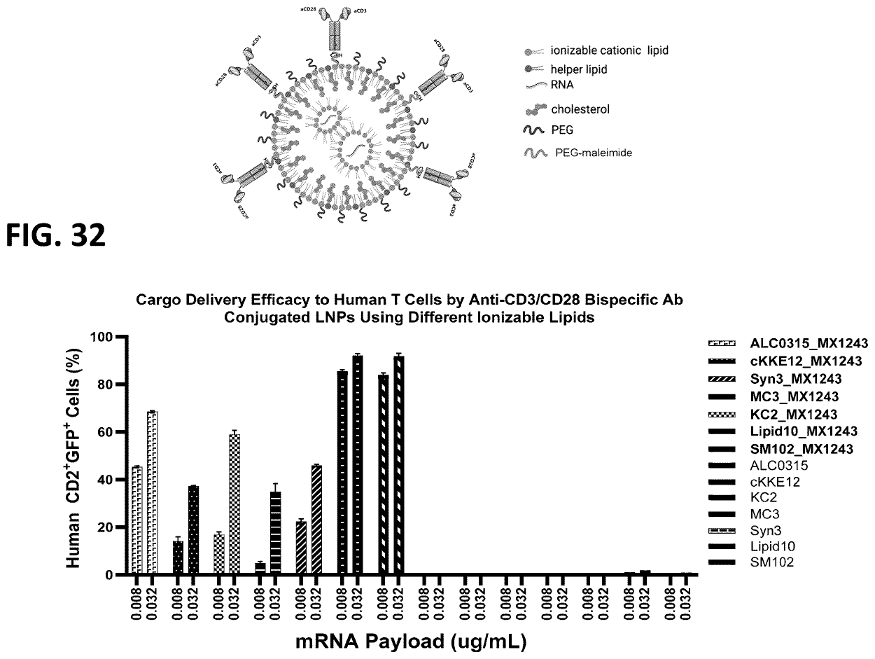

LNPs were prepare using one of several ionizable cationic lipids, a helper lipid, cholesterol, PEG-lipid, an maleimide-PEG-lipid, as shown schematically in FIG. 32, top panel. Five different ionizable cationic lipids were tested: ALC-0315, cKK-E12, Syn3, MC3, KC2, and Lipid 10. mRNA encoding GFP was used as mRNA cargo. Two mRNA payloads were used: 0.008 μg/mL (low dose) and 0.032 μg/mL (high dose=4-fold low dose). The LNP surfaces were decorated with bispecific antibodies against CD3 and CD28. The results showed that the highest expression levels were obtained when using Lipid 10. The antiCD3/antiCD28 LNP containing Lipid 10 transfected human CD2+ T cells at a higher rate than any of the other ionizable cationic lipids used. Over 80% of the T cells incubated with the antiCD3/antiCD28 LNP containing Lipid 10 expressed GFP. Even at the lower dose, the antiCD3/antiCD28 LNP containing Lipid 10 performed better than any of the other LNP formulation, even at the high dose. See FIG. 32, bottom panel.

### Example 13

### Targeting Antibody Ratio Optimization

A reduction in the fraction of maleimide-PEG-lipid in the LNP was tested. The molar ratio of maleimide-PEG-lipid was reduced from 0.2 to 0.05, i.e., a 4-fold reduction. In other words, the reduced maleimide LNP has only ¼ of the targeting antibodies. Both LNP preparations had similar potency in CD2+ T cells. FIG. 33, top panel. However, when T cell activation was analyzed, it was observed that the low maleimide LNP resulted in much lower T cell activation. In fact, at the 0.002 μg/mL and 0.008 μg/mL mRNA payloads the T cell activation was negligible. FIG. 33, bottom panel. Thus, reducing the molar ratio of antibodies to 14 of the original ration significantly reduced T cell activation potential without affecting potency.

### Example 14

### Cell-Type Specific LNP Targeting

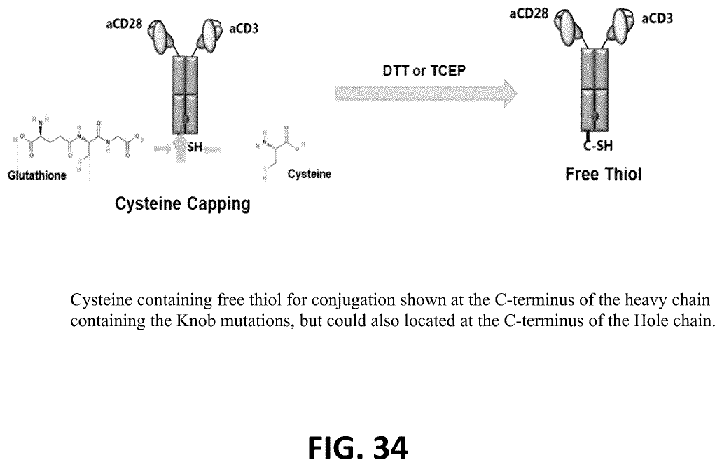

Different targeting antibodies were tested. It was observed that decorating the surface of LNP with antibodies targeting CD3 and CD28 specifically directed the LNP to T cells. On the other hand, when the surface of the LNP was decorated with antibodies specific for CD19, CD79, and CD40L, the LNP were specifically directed to B cells. FIG. 34.

### Example 15

### Development of Production Process for Producing Antibody Conjugated LNP for Targeted Delivery of mRNA Cargo to Human Primary T Cells

One way to achieve targeted delivery using LNP is to decorate the LNP with ligands and/or antibodies that are capable of binding to cell surface proteins and subsequently triggering the endocytosis for internalization and endosomal release of mRNA/DNA cargo. To develop the suitable production process, the following technical aspects were considered:
(a) Identification of suitable ionizable cationic lipid: The preferred ionizable cationic lipid should possess qualities such as low-medium overall delivery capacity in vivo (mouse), low delivery to liver and limited delivery to immune cell types, rigidity/stability for tolerating chemical conjugation and downstream purification processes, high concentration formulation allowing long term storage options. Several ionizable cationic lipids were tested.
(b) Antibody decapping: Process to generate free thiol from the protected thiol group from the engineered cysteine. The design selected was to add one cysteine amino acid to the C-terminus of the heavy chain of a bispecific antibody containing the “Knob” mutations. The process was also used in monoclonal antibodies, or bispecific/multispecific antibodies.
(c) LNP preparation: The step included formulation optimization for targeted delivery to human T cells.
(d) Derivatization: Conjugation chemistry/purification process to produce and achieve good yield and high-quality product.

To produce antibody-conjugated LNP, specific LNP formulations with 5 components (ionizable cationic lipid including MC3, KC2, SM-102, or Lipid 10; DSPC; DMG-PEG2000; cholesterol; and DSPE-PEG2000-maleimide) were prepared using the NanoAssemblr™ Ignite™ system (Cytiva, Wilmington, DE) according to manufacturer's protocol. Briefly, the lipid components were dissolved in ethanol and mRNA was reconstituted in 20 mM sodium citrate (pH4.5), followed by mixing of the organic and aqueous solutions at a ratio of 1:3 at a speed of 10 ml/minute using the Ignite™ system. The resulting LNP were immediately dialyzed against a 200× volume of DPBS (pH7.4), twice, for 2 hours each time. The LNP were concentrated by centrifugation using 100K Amicon Ultra Centrifuge Filters and sterilized using a 0.22 μm syringe filter. Concurrently, antibody decapping was carried out using DTT or TCEP at a DTT(TCEP)/antibody molar ratio of 3 at 37° C. for 2 hours, followed by using a Zaba desalting column to remove the residual DTT/TCEP.

Decapped antibody was mixed with LNP at an antibody/maleimide PEG molar ratio of 0.7 and incubated at 25° C. for 2 hours without shaking, and at 4° C. overnight. A quenching step using 2:1 (N-Acetyl L-Cysteine/maleimide PEG) molar ratio of N-Acetyl L-cysteine was performed at 25° C. for 1 hour followed by further purification using a CL-4B size exclusion column with DPBS buffer plus 2 mM EDTA. The biophysical properties of the LNP and LNP-antibody conjugate were measured using Dynamic Light Scattering (DLS)/Electrophoretic Light Scattering (ELS) (ZETASIZER ULTRA, Malvern, Cambridge, UK), and mRNA content was measured using the Ribogreen assay (Thermo Fisher Scientific, Waltham, MA). See FIG. 33.

### Example 16

### Optimization of Antibody De-Capping Process to Generate Free Thiol from the Protected Thiol Group for the Engineered Cysteine in the C-Terminus of the Antibody

The strategy selected to conjugate targeting molecules (e.g., antibodies) to the surface of LNP relied on maleimide-thiol chemistry. In this approach, a cysteine was added at the C-terminus of a polypeptide chain of the target molecule. In the case of heteromeric bispecific antibodies, the cysteine for conjugation was added at the C-terminus of the heavy chain containing the Knob mutations. By attaching the heavy chain containing the Knob mutations to the surface of the LNP it is possible to control of the specific antibody chain that is attached to the LNP, the site of conjugation, and allow to obtain homogenous site-specific conjugates.

The engineered cysteine is mostly capped by post-translational modifications such as glutathionylation or cysteinylation in protein produced in mammalian cells. It is well documented that free cysteine in antibodies produced in mammalian cells is mostly capped by post-translational modifications such as glutathionylation or cysteinylation in protein produced in mammalian cells (ref). Reducing agents such as tris(2-carboxyethyl)phosphine hydrochloride (TCEP) or dithiothreitol (DTT) can be used to obtain free cysteine residue. See FIG. 34. A de-capping step using DTT or TCEP was used to generate the free thiol group for click chemistry with maleimide on the LNPs. To efficiently de-cap the C-terminus cysteine, and to minimize the disruption of the disulfide bonds in the targeting antibody, optimizations were carried out. Three parameters were optimized: (i) DTT or TCEP/antibody molar ratio, (ii) temperature, and (iii) reaction time.

TABLE 19
Optimization of de-capping condition using DTT.

| Temperature | Mole ratio between | C-terminal |
|---|---|---|
| (° C.) | DTT and Ab | cysteine |
| 37 | 5:1 | De-capped |
| 37 | 1:1 | No |
| 25 | 5:1 | Partially de-capped |
| 25 | 1:1 | No |
| 4 | 5:1 | No |
| 4 | 1:1 | No |

1 ml of bispecific antibody MX1500 (1 mg/ml) in PBS was incubated with DTT at molar ratios of 5:1 and 1:1 (DTT/antibody) at the indicated temperature for one hour, followed by buffer exchange against PBS to remove excess DTT. 0.3 mg from each sample was used to react with equal molar amounts of the DSPE-PEG4-maleimide lipid at 37° C. for 2 hours. The resulting samples were precipitated in 90% ethanol at 4° C. The pellet was reconstituted in PBS and then analyzed by LC-MS to quantitate percentage of DSPE-PEG4-maleimide conjugation to free cysteine.

TABLE 20
Optimization of de-capping condition using TCEP.

| Temperature | Mole ratio between TCEP | C-terminal |
|---|---|---|
| (° C.) | and Ab¹ | cysteine² |
| 37 | 5:1 | ~94% reduced |
| 37 | 3:1 | ~92% reduced |
| 37 | 1:1 | Not reduced |
| 4 | 5:1 | ~93% reduced |
| 4 | 3:1 | ~92% reduced |
| 4 | 1:1 | Not reduced |

¹MX1500 dimer was used for experiments.
²Percentage of reduced antibody was estimated based on corresponding ion intensity.

1 ml of bispecific antibody MX1500 (1 mg/ml) in PBS was incubated with TCEP at molar ratios of 5:1 and 1:1 (TCEP/antibody) at the indicated temperature for one hour, followed by buffer exchange against PBS to remove excess TCEP. 0.3 mg from each sample was used to react with equal molar amounts of the DSPE-PEG4-maleimide lipid at 37° C. for 2 hours. The resulting samples were precipitated in 90% ethanol at 4° C. The pellet was reconstituted in PBS and then analyzed by LC-MS to quantitate percentage of DSPE-PEG4-maleimide conjugation to free cysteine. De-capping using DTT was effective when using DTT/antibody molar ratios between 3:1 and 5:1 at 37° C. with shaking (350 rpm) for one hour. De-capping using TCEP was effective when using a TCEP/antibody molar ratio of 3:1 at temperatures from 4-37° C. with shaking (350 rpm) for one hour. Lower DTT or TCEP to antibody molar ratios and lower temperatures were preferred to minimize the reduction of disulfide bonds in the antibody. An essential step in the de-capping process is to remove 99.99% of the unreacted DTT or TCEP post de-capping step due to potential interaction between DTT or TCEP with the maleimide of the DSPE-PEG4-maleimide, potentially resulting in poor derivatization rates or inconsistent derivatization.

### Example 17

### LNP Formulation Optimization for Antibody Conjugation

The potential major contributor of adverse events in CAR T cell therapy is the induction of cytokine release. Accordingly, a key attribute of a viable product for in vivo CAR-T production using antiCD3/antiCD28 targeting is having a LNP formulation capable of achieving efficient gene delivery at a low mRNA payload dose. In other words, the system should be able to efficiently deliver the mRNA payload while keeping the amounts of molecules that can trigger an immune response, e.g., the mRNA payload and the targeting antibodies as low as possible. The development/optimization of an LNP formulation suitable for targeting antibody conjugation and payload delivery includes, e.g., (i) selection of ionizable cationic lipid (ilipid); (ii) molar percentage (molar %) of ionizable cationic lipid optimization; (iii) molar percentage of maleimide-PEG optimization; (iv) N/P ratio (the stoichiometry of protonatable nitrogen (N) and anionic phosphate groups (P) in a nucleic acid); and, (v) molar percentage of helper lipid optimization.

The optimization of the molar percentage (molar %) of ionizable cationic lipid is important to achieve high potency. The optimization of molar percentage of maleimide-PEG is important to fine-tune the density of targeting antibody on the surface of the LNP, and at the same time reduce potential toxicity. The optimization of the N/P ratio is important to achieve optimal transfection by having an adequate total lipid to nucleic acid payload ratio. The optimization of the molar percentage of helper lipid is important for the stability of the LNP and also for endosomal escape.

The overarching goal was developing an antibody-conjugated LNP drug candidate possessing the highest gene delivery efficacy while using the lowest quantity of targeting antibody (anti-CD3/CD28), and having an antibody to payload ratio lower than 1 (<1) (w/w). The distinct phases of the LNP optimization process are described in detail in the examples below. The optimization process resulted in an optimized LNP formulation comprising the Lipid 10 ionizable cationic lipid, a bispecific antiCD3/CD28 targeting antibody conjugated to the surface of the LNP, and an mRNA payload. The optimized LNP system had an N/P ratio of 7 and the following lipid composition (amount in % of molar ratio):Lipid 10 27.5%, DSPC 16%, cholesterol 54%, DMG-PEG2000 2.45%, DSPE-PEG2000-maleimide 0.05%. These LNP can be conjugated to an antibody (e.g., a bispecific antiCD3/CD28 targeting antibody) or two a pair of antibodies (e.g., an antiCD3 antibody and an antiCD238 antibody). Thus, after antibody conjugation, the resulting LNP would be Lipid 10 27.5%, DSPC 16%, cholesterol 54%, DMG-PEG2000 2.45%, DSPE-PEG2000-maleimide-antibody 0.05%.

### Example 18

### LNP Formulation Optimization: Ionizable Cationic Lipid (Ilipid) Selection and Optimization of Ilipid %

The ideal ionizable cationic lipid (ilipid) should have minimal liver delivery capacity and low overall delivery potential to major organs such as lung, kidney, heart, spleen, brain, but show high delivery efficiency to intended target cells/tissue as an antibody conjugated LNP product at low dosing with acceptable toxicity profile. The ilipid should also possess stability profile for industrial scale manufacturing, tolerating chemical conjugation and downstream purification processes, and suitable for final storage buffer formulation allowing long term storage options. A collection of commercially available ilipids (cKK-E12, MC3, SM-102, ACL-0315, KC2, Lipid A6, Lipid M, Lipid 10, C14-4) was tested in formulation of 50:10:43.5:1.3:0.2 or 35:16:46.5:2.3:0.2 (ilipid:DSPC:Chol:PEG2000-DMG:DSPE-PEG2000-Maleimide) by conjugating to anti-CD3/CD28 bispecific antibodies. Lipid 10 was chosen for further production optimization due to its extrahepatic tropism, high delivery efficiency to human primary T cells and good overall recovery rate post conjugation/purification. Next, the molar percentage (molar %) of Lipid 10 was optimized using the ranges showed in TABLE 21.

TABLE 21
In this workflow, the molar % of the Lipid 10 varied
from 20-35%, while the Helper lipid (DSPC, 16%),
PEG2000-DMG (2.3%), DSPE-PEG2000-Maleimide (0.2%),
and Cholesterol varied accordingly.

| Name | Molar ratio (%) |
|---|---|
| Cholesterol | 46.5-61.5 |
| DSPE-PEG2000-Maleimide | 0.2 |
| DSPC | 16 |
| Lipid 10 | 20-35 |
| DMG-PEG2000 | 2.3 |

To produce antibody conjugated LNPs, LNPs in specific formulation with 5 components (Lipid 10, DSPC, DMG-PEG2000, Cholesterol, and DSPE-PEG2000-Maleimide) were prepared using the NANOASSEMBLR™ IGNITE™ system according to manufacturer's protocol. Briefly, the lipid components were dissolved in ethanol, and mRNA was reconstituted in 20 mM sodium citrate (pH4.5), followed by mixing of the organic and aqueous solutions at a ratio of 1:3 and a speed of 10 ml/minute using the IGNITE™ system. The prepared LNPs were immediately dialyzed against 200× volume of DPBS (pH7.4) twice for 2 hours each time. The LNPs were concentrated by centrifugation using 100K Amicon Ultra Centrifuge Filters and sterilized using a 0.22 μm syringe filter. Concurrently, antibody decapping was carried out using DTT or TCEP at DTT(TCEP)/antibody molar ratios of 3 at 37° C. for 2 hours followed by using a Zaba desalting column to remove the residual DTT/TCEP.

Decapped Ab was then mixed with LNP at an antibody to maleimide-PEG-lipid molar ratio of 0.7, and incubated at 25° C. for 2 hours without shaking and at 4° C. overnight. A quenching step using a 2:1N-Acetyl L-Cysteine:maleimide-PEG-lipid molar ratio was performed at 25° C. for 1 hours and followed by further purification using a CL-4B size exclusion column with DPBS buffer plus 2 mM EDTA. The biophysical properties of the LNPs and LNP-Antibody were measured using Dynamic Light Scattering (DLS)/Electrophoretic Light Scattering (ELS) and mRNA content was measured using Ribogreen assay.

To transfect unstimulated human PBMCs using conjugated LNPs, the incubation of LNP-antibody conjugates with PBMCs was set as following: [1] Cryovials containing samples from two Human PBMC donors were taken from cryofreezer and immediately thawed in the 37° C. water bath; [2] the thawed contents of the cryovials were transferred to 9 mL of pre-warmed RPMI and pelleted by centrifugation at 400×g for 5 minutes at room temperature; [3] the supernatant was removed and replaced with 10 mL of pre-warmed RPMI; [4] the PBMCs were pelleted by centrifugation at 400×g for 5 minutes at room temperature; [5] the supernatant was removed and replaced with 10 mL of pre-warmed RPMI; [6] the PBMCs were rested in an incubator at 37° C. and 5% CO₂ for 2 hours; [7] the PBMCs were then pipetted through a 70 μM strainer (Falcon, Cat. No. 352350) and counted using Trypan blue (Invitrogen, Countess 3 FL); [8] PBMCs were diluted to 6.6×10⁵ cells/mL in warm RPMI and then 100,000 PBMCs (150 μL) were added to each well of a 96-well round bottom plate; [9] a separate 96-well round bottom plate was used to serially dilute a commercial antiCD3 monoclonal antibody (Biolegend, Cat No. B301770) as well as the LNPs; [10] the antiCD3 monoclonal antibody and all LNPs were diluted to either 2 nM or 2 μg/mL mRNA payload respectively in 360 μL and serially diluted 4-fold (90 μL+270 μL media) in RPMI (Gibco, Cat. No. 72400-047) supplemented with 10% FBS (Gibco, 16140-089) and 1% Pen/Strep (Gibco, Cat. No. 15140-122); [11]50 μL of the serially diluted antiCD3 and/or LNPs were added to their designated wells; [12]5 μL of 0.1 mg/mL diluted stock of antiCD28 (Biolegend, Cat. No. B306300) was added to the wells that contained soluble antiCD3; and [13] the plate was incubated at 37° C., in 5% CO₂ for 20 hours.

Cell staining for Flow Cytometry Analysis: After the 20-hour incubation time, all well contents were transferred from the flat bottom 96-well plate to a V-bottom 96-well plate and centrifuged at 400×g for 5 minutes at room temperature. [1] the supernatant was removed, and the cells were washed with 200 μL PBS; [2] the cells were pelleted by centrifugation at 400×g for 5 minutes at room temperature; [3] the supernatant was removed and 100 μL of staining master mix in PBS was added. The mix comprises viability dye (1/1000, Invitrogen, eFluor 506, Cat. No. 65086618), CD69-PerCP-Cy5.5 (1/100, Biolegend, Cat. No. 310926), CD2-APC/C7 (1/100, Biolegend, Cat. No. 300220), CD4-BV605 (1/100, Biolegend, Cat. No. 317438), and CD8-PE (1/100, Biolegend, Cat. No. 301008); [4] the plate was incubated for 25 minutes at 4° C. in the dark; [5] cells were washed with 150 μL FACS buffer (1×PBS, 1% Bovine Serum Albumin (Fisher, Cat. No. BP1600-1), 0.1% Sodium Azide (Ricca, Cat. No. 7144.8-16); [6] the cells were pelleted by centrifugation at 400×g for 5 minutes at room temperature; [7] the supernatant was removed, the cells were washed with 200 μL FACS buffer and centrifuged at 400×g for 5 minutes at room temperature; [8] the supernatant was removed, and the cells were suspended in 150 μL FACS buffer; [9] flow cytometry data acquisition was performed on an Attune flow cytometer; each flow cytometry sample has a volume of 125 μL and was acquired with a stop gate set for 5,000 Live CD2+ Cells.

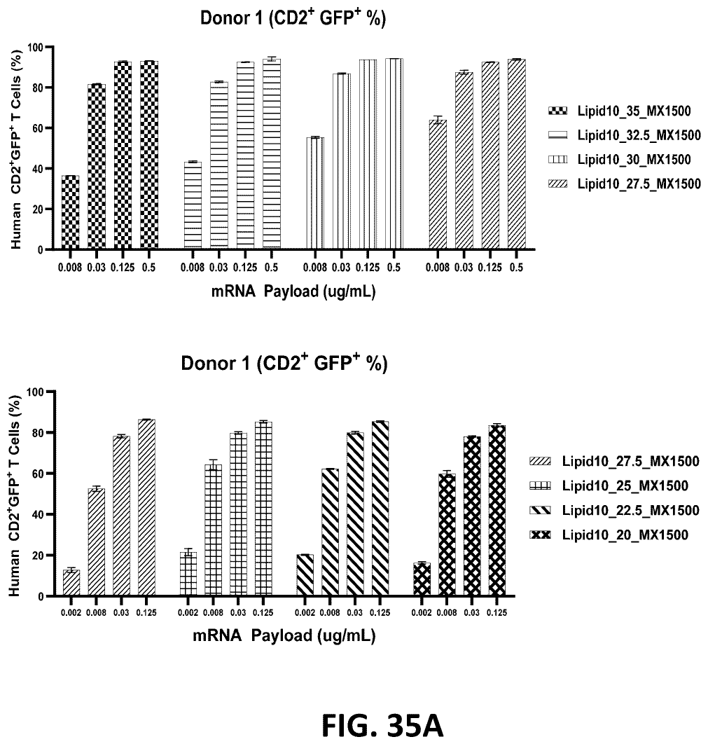

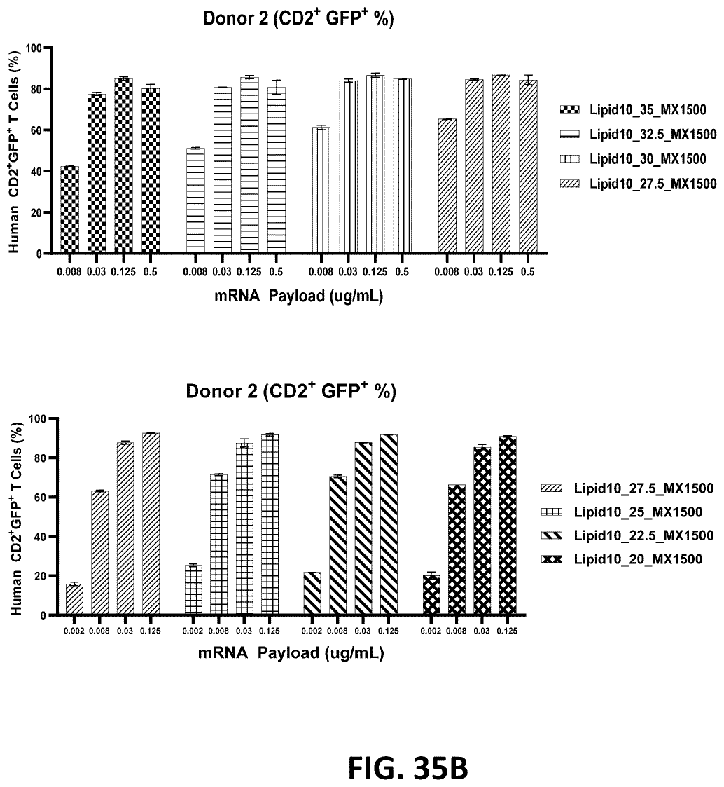

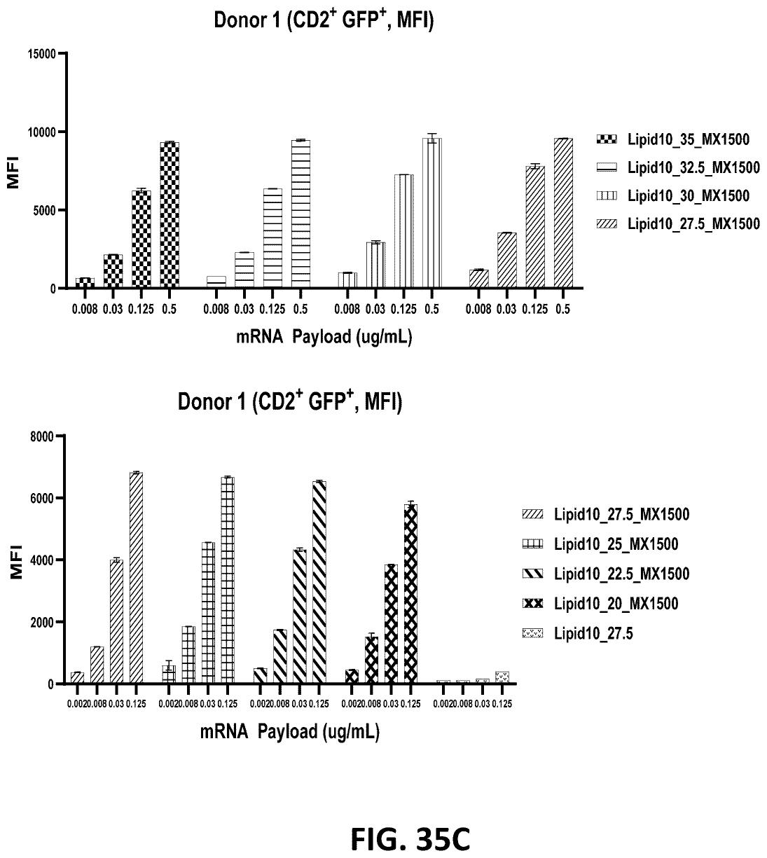

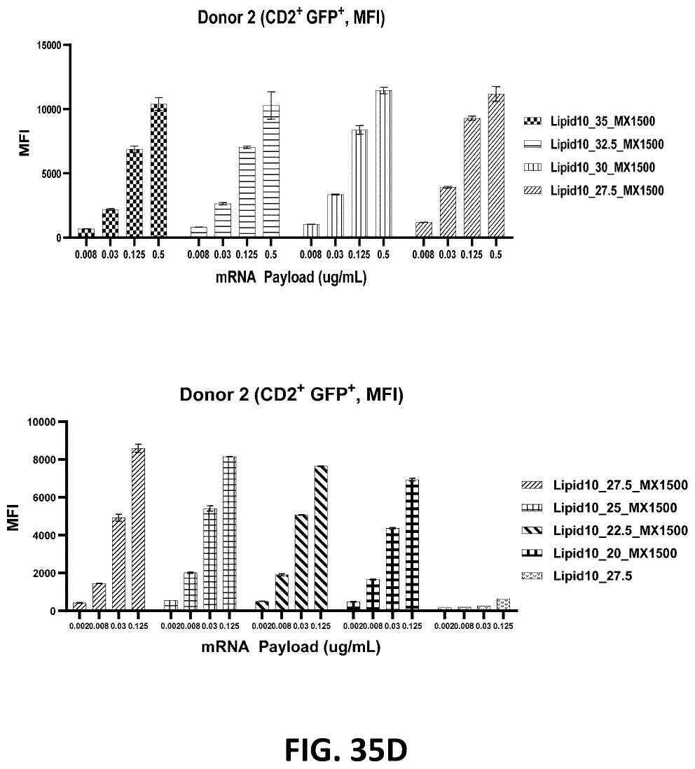

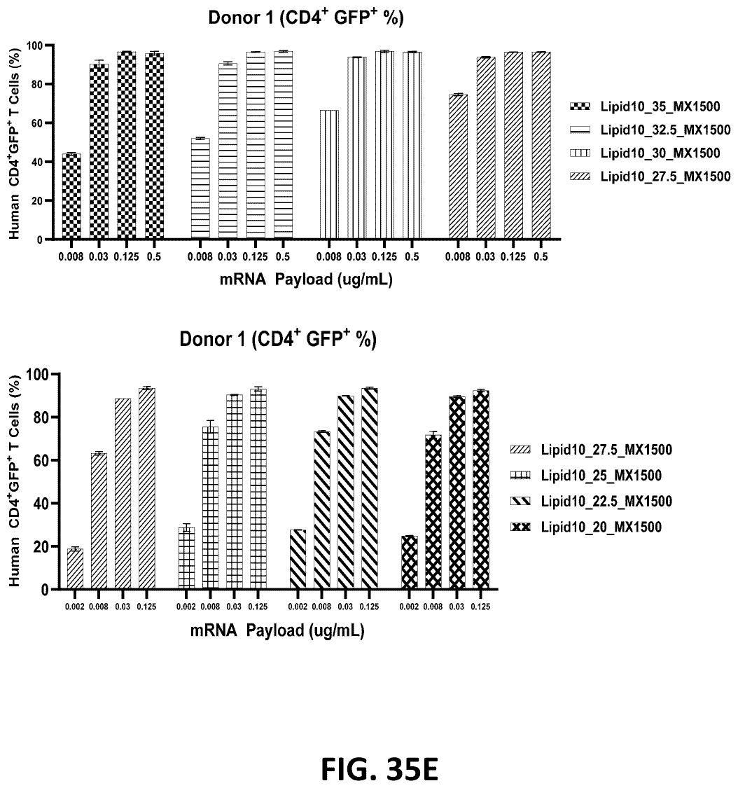

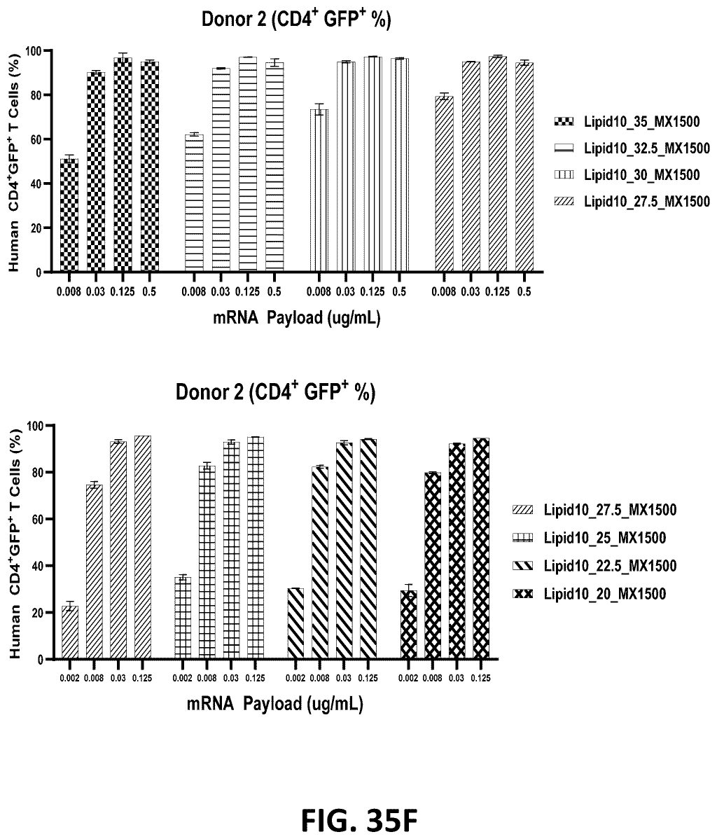

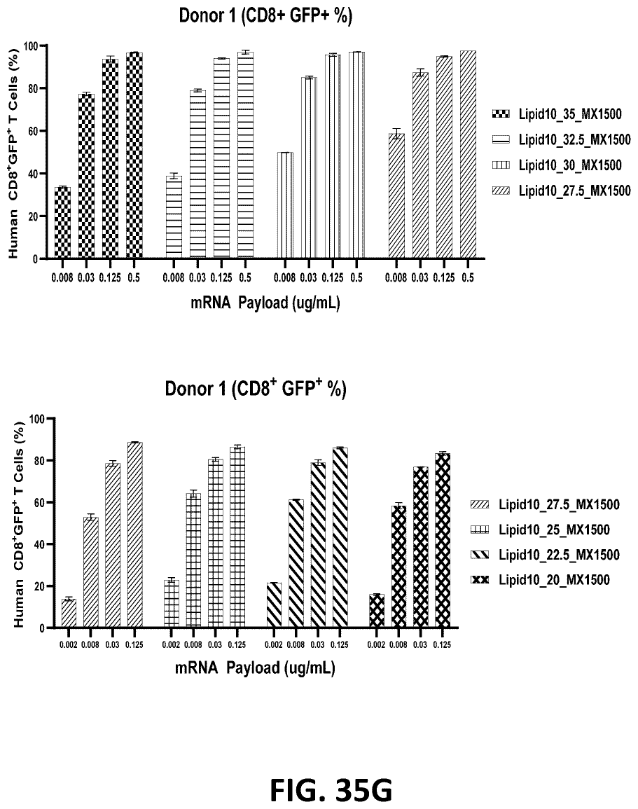

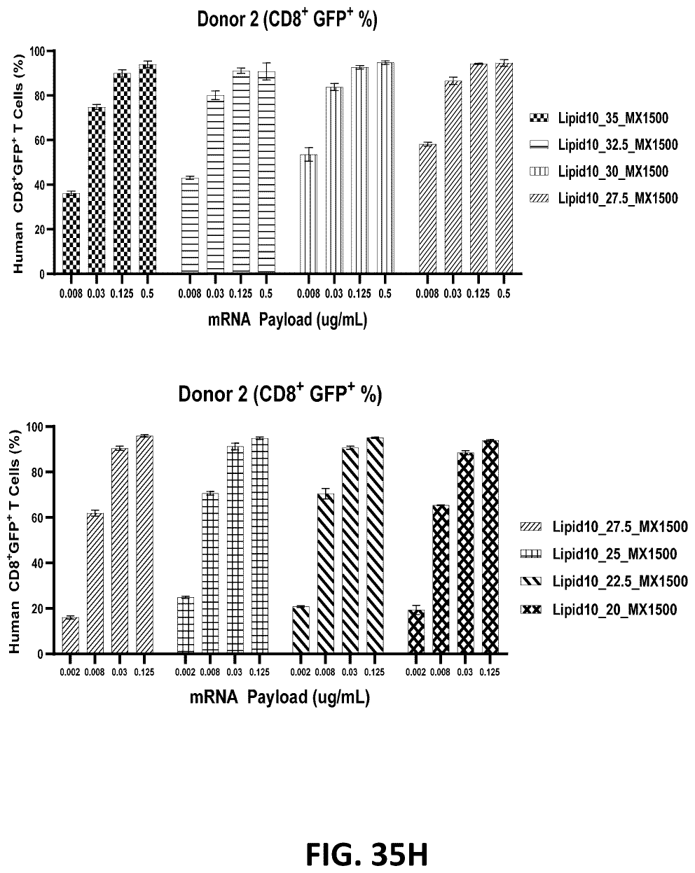

Results are shown in FIGS. 35A, 35B, 35C, 35D, 35E, 35F, 35G, and 35H. The biophysical properties of the unconjugated and conjugated LNPs are summarized in TABLE 22.

TABLE 22
Biophysical properties of unconjugated and conjugated LNPs with various molar
% of Lipid 10. Measurements were taken as described above using ZETASIZER
ULTRA. LNPs were diluted 50X in DPBS for size, PI, and charge measurements.

| | | Polydispersity | | mRNA Encapsulation | mRNA Recovery |
|---|---|---|---|---|---|
| LNP | Size (nm) | Index (PI) | Charge (mV) | Efficiency (%) | Rate (%) |
| Lipid10_27.5_0.20 | 78 | 0.03 | −3.28 | 99 | 65.9 |
| Lipid10_27.5_0.20_MX1500 | 101 | 0.15 | −0.93 | 97 | 60.2 |
| Lipid10_25_0.20 | 77 | 0.08 | −2.52 | 99 | 68.4 |
| Lipid10_25_0.20_MX1500 | 104 | 0.12 | −1.67 | 97 | 61.1 |
| Lipid10_22.5_0.20 | 77 | 0.06 | −1.76 | 99 | 62.4 |
| Lipid10_22.5_0.20_MX1500 | 101 | 0.10 | −0.76 | 97 | 69.7 |
| Lipid10_20_0.20 | 78 | 0.08 | −3.05 | 99 | 59.6 |
| Lipid10_20_0.20_MX1500 | 100 | 0.11 | −1.11 | 97 | 73.9 |
| Lipid 10_27.5 | 72 | 0.07 | −1.84 | 99 | 75 |
| Lipid 10_27.5_MX1500 | 92 | 0.12 | −1.01 | 98 | 74 |
| Lipid 10_30 | 72 | 0.02 | −3.53 | 99 | 81 |
| Lipid 10_30_MX1500 | 89 | 0.08 | −1.25 | 98 | 62 |
| Lipid 10_32.5 | 71 | 0.05 | −2.69 | 99 | 76 |
| Lipid 10_32.5_MX1500 | 88 | 0.10 | −1.6 | 98 | 69 |
| Lipid 10_35 | 71 | 0.04 | −0.48 | 99 | 96 |
| Lipid 10_35_MX1500 | 85 | 0.09 | 1.24 | 99 | 60 |
| Lipid 10_37.5 | 77 | 0.02 | −1.67 | 99 | 79 |
| Lipid 10_37.5_MX1500 | 87 | 0.03 | −1.83 | 96 | 43 |

For in vivo assessment of delivery efficiency, a human PBMC reconstituted NSG mouse model was used. Briefly, NSG or NSG-MHC I/II DKO mutant mice were purchased from the Jackson Laboratory. Frozen PBMCs from selected human donors (Research Blood Components, Cat. No. (002)) were quickly thawed at 37° C. and transferred to a 15 mL Falcon tube containing 9.5 mL of pre-warmed media (RPMI1640+10% FBS).

Cell suspensions were gently mixed and centrifuged at 250×g for 8 minutes. Cells were resuspended in fresh pre-warmed media, counted (Invitrogen, Countess 3 FL) and rested for 1 hour at 37° C., 5% CO2 incubator in media. Cultured cells were then re-suspended and strained using a 70 m cell strainer (Falcon, Cat. No. 352350). Cells were then pelleted at 350×g for 5 minutes, counted (Invitrogen, Countess 3 FL), and resuspended to 2×10⁶/mL in 6-well dishes. Cell cultures were stimulated with DYNABEADS™ Human T-Activator CD3/CD28 for T Cell Expansion and Activation (Thermofisher, Cat. No. 11161D) at 1:10 Bead/PBMC ratio and incubated at 37° C., 5% CO2 for 2 days. Cultures were transferred to 15 mL Falcon tubes and Dynabeads were removed through use of a cell culture magnet (Invitrogen, DYNAL). Cell suspensions were strained using a 70 μm cell strainer (Falcon, Cat. No. 352350), pelleted by centrifugation at 350×g for 5 minutes and resuspended in 10 mL 1×PBS. Cells were counted and adjusted to 25×10⁶/mL in PBS (pH 7.4). 10×10⁶ cells were intraperitoneally injected into each NSG-MHC I/II DKO mouse (Jackson, Strain #:025216). The status of human T cell presence in mouse blood was monitored weekly starting from week 2 post reconstitution. Reconstituted mice with human T cell range from 10% to 50% were selected for LNP delivery studies between 3-10 weeks post reconstitution. Selected formulations of conjugated Lipid 10 LNPs were injected into mice intravenously at indicated dose/mouse (0.2 g, 0.6 g, or 2 g/mouse). 50 μL to 100 μL of blood from each mouse was drawn between 16 and 20 hours post-delivery, and transferred to 1.5 mL Eppendorf tube. Blood was then resuspended in 500 μL of 1×PBS and pelleted by centrifugation at 500×g for 5 minutes. This step was then repeated. Cell pellets were then resuspended in 100 μL diluted Viability dye (1/1000 stock in 1×PBS, Invitrogen, eFluor 506, Cat. No. 65086618) and incubated for 30 minutes covered from light at 4° C. During incubation, a master mix of staining antibodies (mCD45-PE-Cy7, Biolegend, Cat. No. 103113; mCD11b-BV421, Biolegend, Cat. No. 101235; CD2-APC/C7, Biolegend, Cat. No. 300220 was made in FACS buffer (1×PBS, supplemented with 1% Bovine Serum Albumin (Fisher, Cat. No. BP1600-1) and 0.1% sodium azide (Ricca, Cat. No. 7144.8-16). 100 μL of the isotype control stain or master mix was added to each sample and cell suspensions were gently mixed. While covered from light, cell suspensions were incubated at 4° C. for 60 minutes. Samples were then washed with 500 μL of FACS buffer and pelleted by centrifugation at 500×g for 5 minutes. This step was then repeated. Cell pellets were gently resuspended in 1.0 mL of ACK Lysis Buffer (Gibco, Cat. No. A1049201) and pelleted by centrifugation at 500×g for 5 minutes. Cell pellets were washed by gentle resuspension in 500 μL of FACS buffer and pelleted by centrifugation at 500×g for 5 minutes. This step was then repeated. Cell pellets were then resuspended in 180 μL FACS buffer, and 25 μL of Precision Counting Beads (Biolegend, Cat. No. 424902) were added to each sample. Data acquisition was performed on an Attune flow cytometer. 125 μL of each sample was acquired.

Summary: LNP formulations at fixed molar % of DSPC (16%) and PEG-lipid/Maleimide-PEG-lipid (2.3/0.2%), molar % reduction of Lipid 10 (from 35% to 20%), and molar % increase of cholesterol (from 46.5% to 61.5%) correlated with increased in vitro delivery efficacy to primary human T cells. The best efficacy was observed using 25% of Lipid 10 and 56.5% of cholesterol (FIGS. 35A-35H).

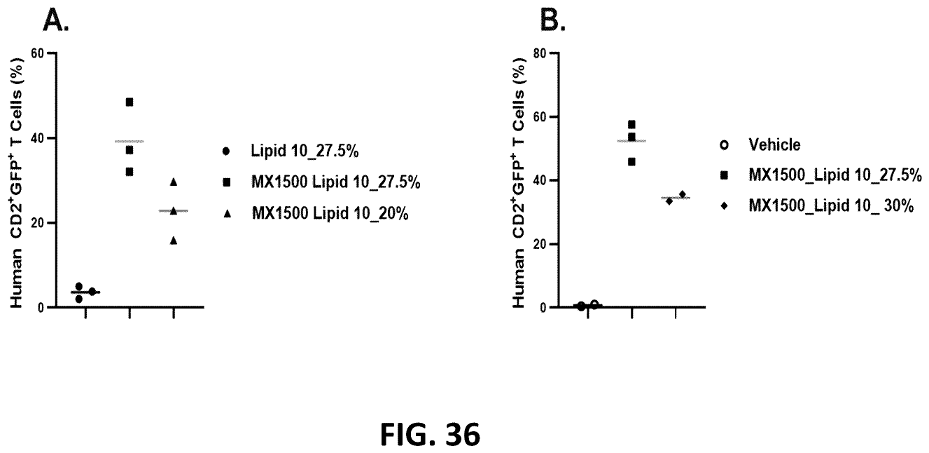

This trend was confirmed in vivo by intravenous delivery of Lipid 10 LNPs using a human PBMCs humanized NSG mouse model (FIG. 36), demonstrating the best in vivo delivery efficacy at 27.5:16:54:2.45:0.05 (Lipid 10:DSPC:Cholesterol:PEG2000-DMG:DSPE-PEG2000-Maleimide) for EGFP mRNA. Among all formulations tested, their biophysical properties (size, PDI, zeta potential, encapsulation efficiency) remained good quality readings (TABLE 22). Molar % reduction of Lipid 10 (from 35% to 20%) and molar % increase of cholesterol (from 46.5 to 61.5%) also resulted in improved yield of the antibody conjugated LNPs (improved recovered rate) (TABLE 22).

### Example 19

### LNP Formulation Optimization: Maleimide-PEG (DSPE-PEG2000-Maleimide)

TABLE 23
In this workflow, the molar % of the DSPE-PEG2000-Maleimide
varied from 0.05-0.2%, while the Lipid 10 (27.5%), Helper
lipid (DSPC, 16%), Cholesterol (54%) are fixed but DMG-
PEG2000 (total PEG 2.5%) varied accordingly.

| Name | Molar ratio (%) |
|---|---|
| Cholesterol | 54 |
| DSPE-PEG2000-Maleimide | 0.05-0.2 |
| DSPC | 16 |
| Lipid 10 | 27.5 |
| DMG-PEG2000 | 2.45-2.3 |

TABLE 24
Biophysical properties of unconjugated and conjugated LNPs with the molar % of the DSPE-PEG2000-
Maleimide varied from 0.05-0.2%. Measurements were taken as described above in previous method
using ZETASIZER ULTRA. LNPs were diluted 50X in DPBS for size, PI, and charge measurements.

| | | Polydispersity | | mRNA Encapsulation | mRNA Recovery |
|---|---|---|---|---|---|
| LNP | Size (nm) | Index (PI) | Charge (mV) | Efficiency (%) | Rate (%) |
| Lipid10_27.5_0% Mal | 78 | 0.04 | −2.5 | 99 | 59.5 |
| Lipid10_27.5_0% Mal_MX1500 | 81 | 0.09 | −3.71 | 97 | 56.3 |
| Lipid10_27.5_0.05% Mal | 83 | 0.07 | −2.51 | 99 | 52.1 |
| Lipid10_27.5_0.05% Mal_MX1500 | 89 | 0.10 | −1.65 | 99 | 73.6 |
| Lipid10_27.5_0.10% Mal | 82 | 0.05 | −1.61 | 99 | 66.6 |
| Lipid10_27.5_0.10% Mal_MX1500 | 92 | 0.07 | −0.26 | 98 | 69.8 |
| Lipid10_27.5_0.15% Mal | 80 | 0.04 | −2.27 | 99 | 56.3 |
| Lipid10_27.5_0.15% Mal_MX1500 | 98 | 0.10 | −2.12 | 97 | 69.9 |
| Lipid10_27.5_0.20% Mal | 78 | 0.03 | −3.28 | 99 | 65.9 |
| Lipid10_27.5_0.20% Mal_MX1500 | 101 | 0.15 | −0.93 | 97 | 60.2 |

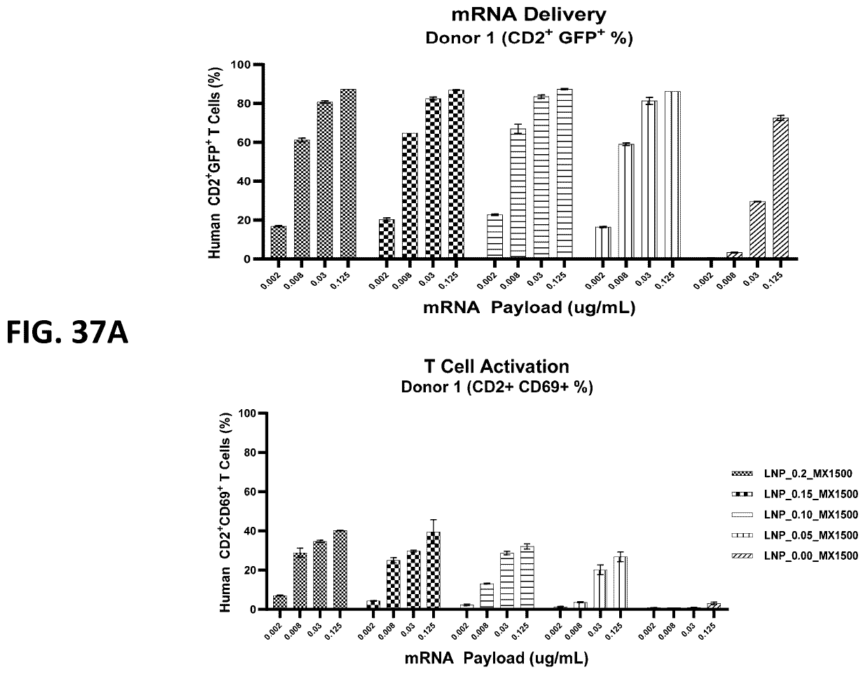

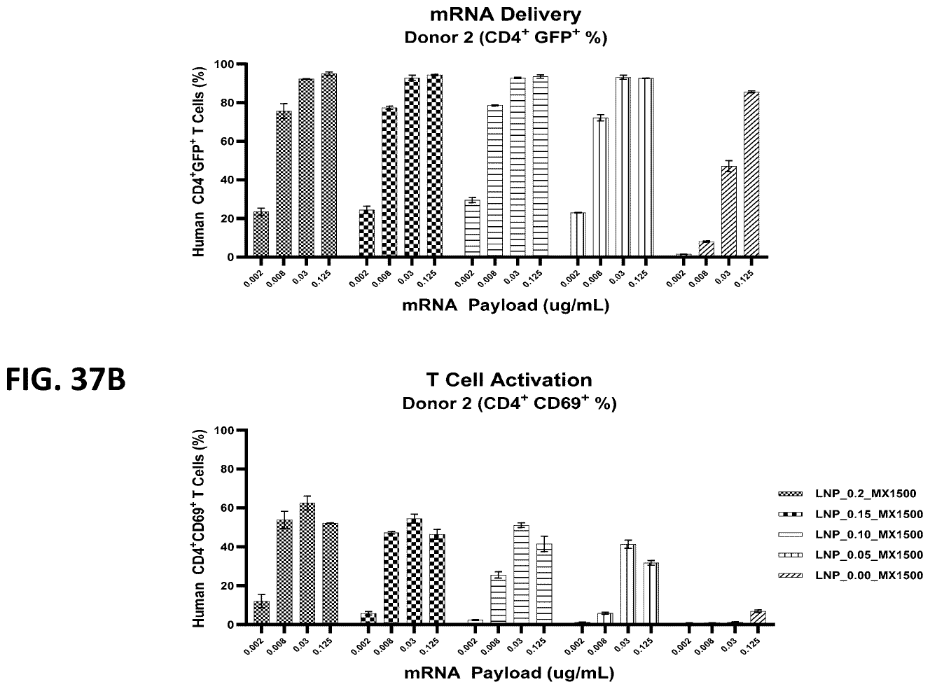

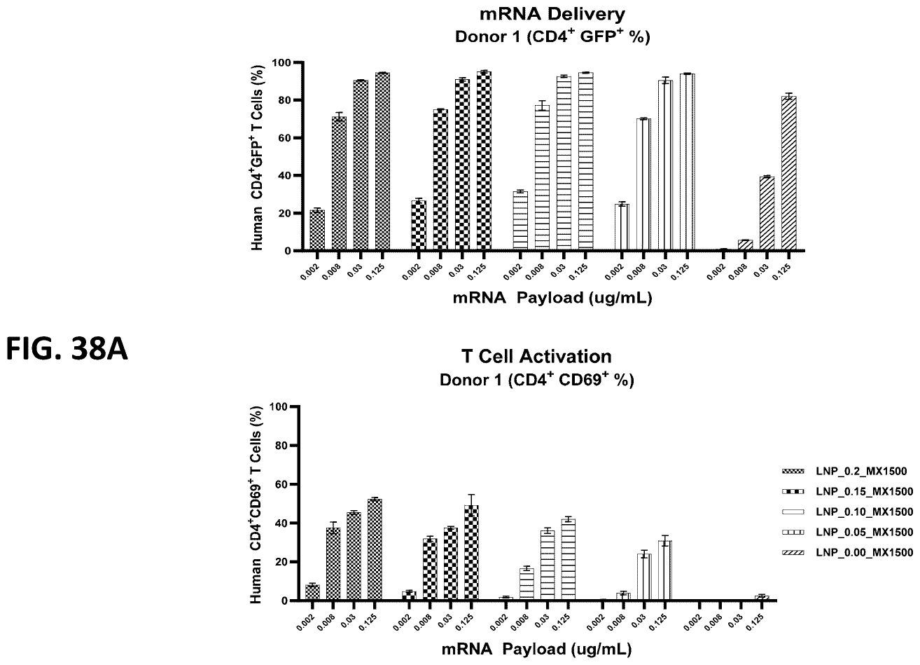

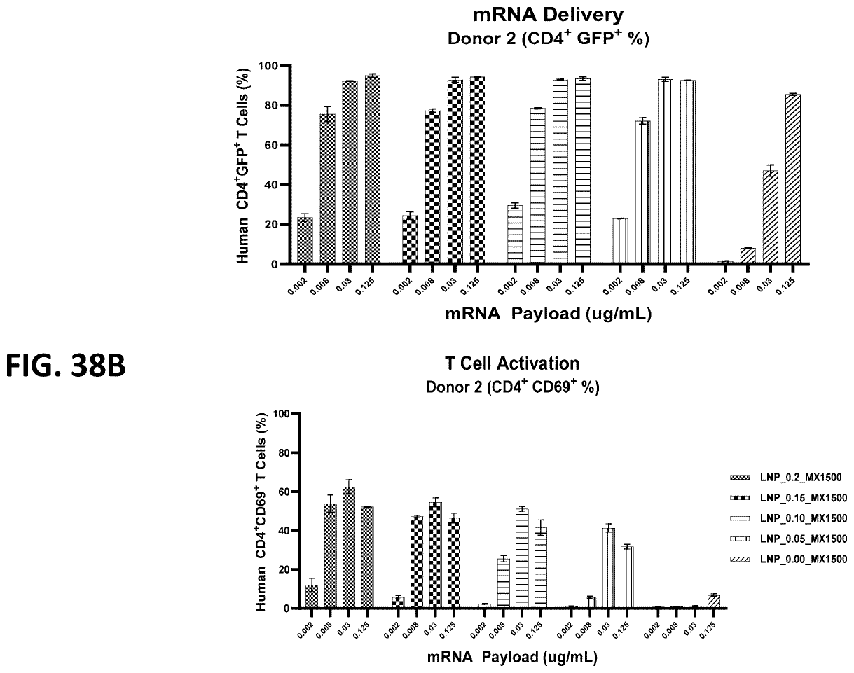

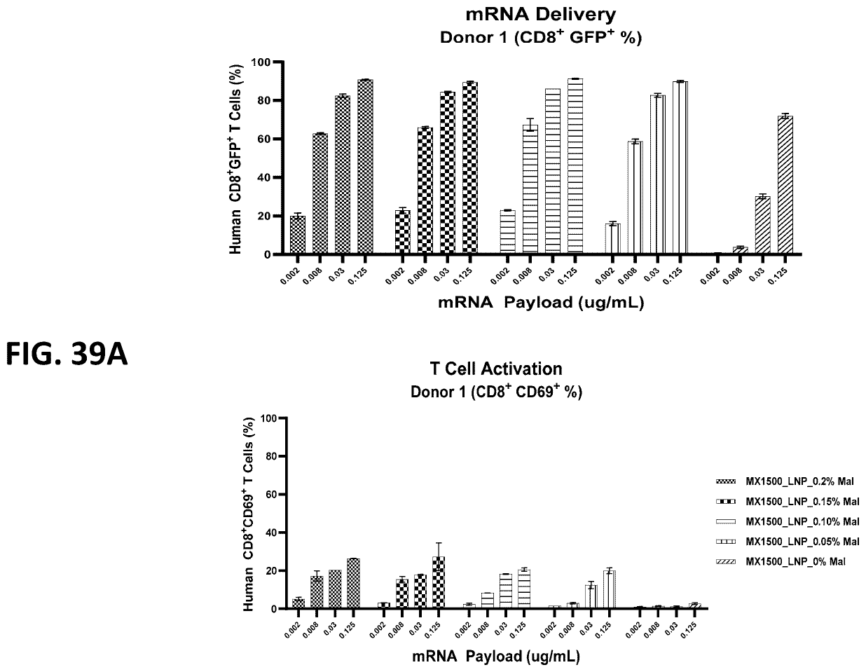

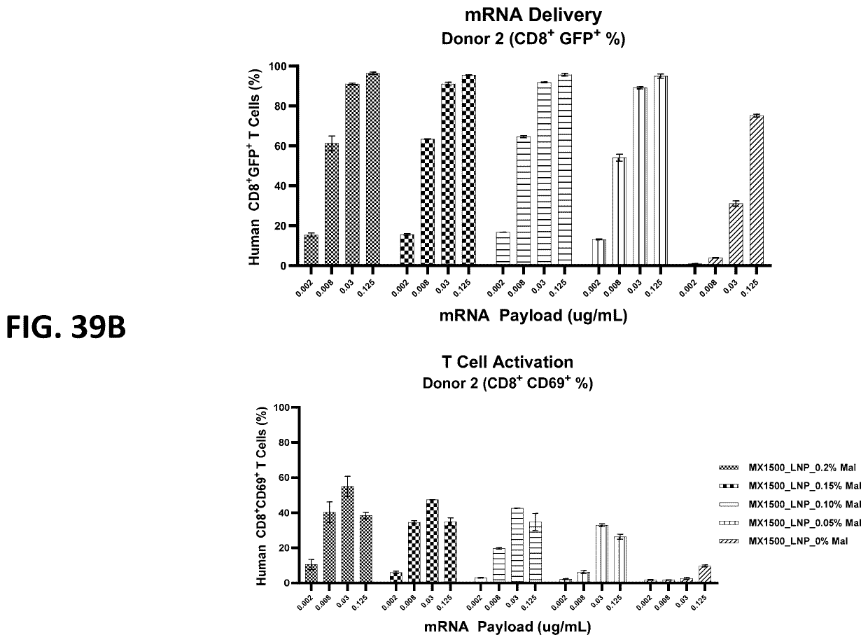

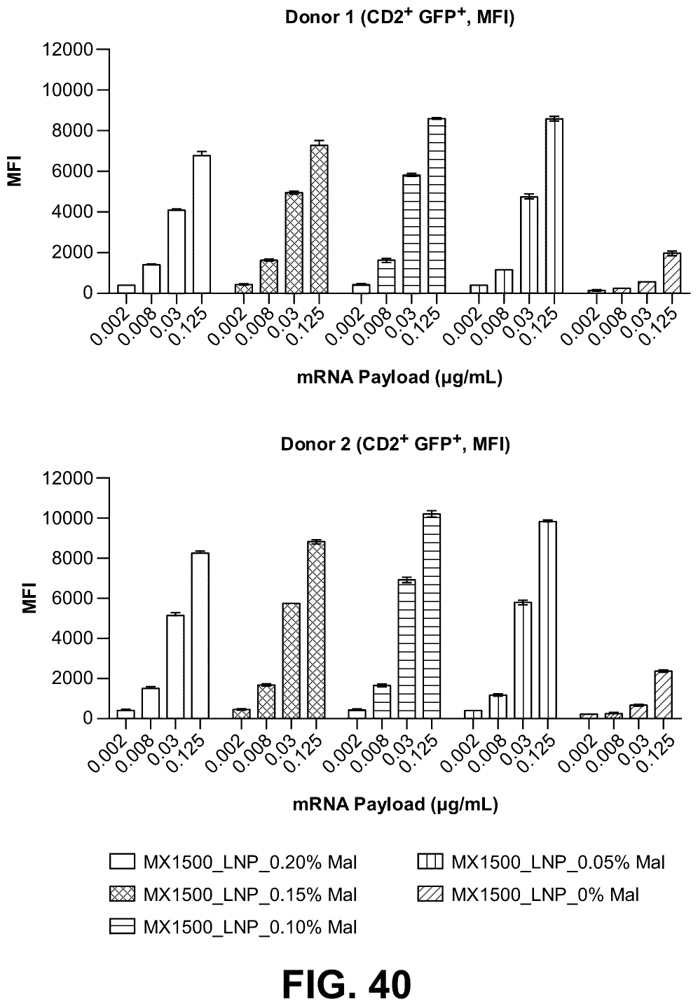

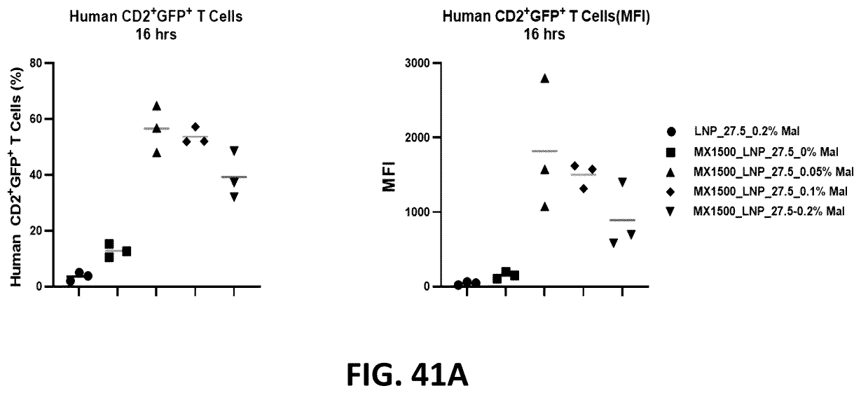

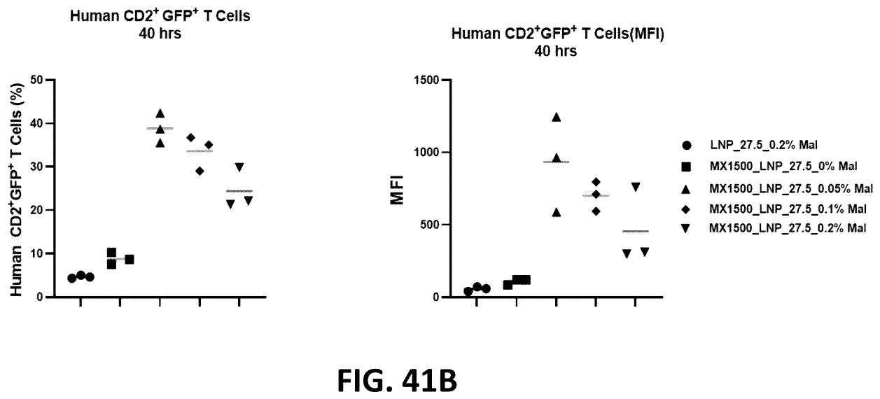

Summary: Reduction of maleimide-PEG lipid (DSPE-PEG2000-Maleimide) correlated with increased in vitro human primary T cell delivery efficiency, which plateaued at 0.1%. See FIGS. 37A-37B, FIGS. 38A-38B, FIGS. 39A-39B and FIG. 40. Reduction of maleimide-PEG lipid (DSPE-PEG2000-Maleimide) also resulted in lower level of T cell activation in vitro, a source of potential adverse events. See FIGS. 37A-37B, FIGS. 38A-38B, and FIGS. 39A-39B. This trend was confirmed in vivo by intravenous delivery of Lipid 10 LNPs using a human PBMCs humanized NSG mouse model (FIGS. 41A-41B), although the best in vivo human T cell delivery efficacy was seen using 0.05% of the DSPE-PEG2000-Maleimide.

### Example 20

### LNP Formulation Optimization: Helper Lipid (DSPC)

TABLE 25
In this workflow, the molar % of the DSPC, Cholesterol,
DMG-PEG2000 and DSPE-PEG2000-Maleimide varied accordingly,
while the Lipid 10 (25%) was constant.

| Name | Molar ratio (%) |
|---|---|
| Cholesterol | 56.5 or 46.5 |
| DSPE-PEG2000-Maleimide | 0.1 or 0.2 |
| DSPC | 16 or 26 |
| Lipid 10 | 25 |
| DMG-PEG2000 | 2.4 or 2.3 |

TABLE 26
Formulations tested included DSPC at 16% versus 26%, with 0.1% and 0.05% of DSPE-PEG2000-Maleimide.

| | | | | PEG Lipids | | | | | | Ionizable |
|---|---|---|---|---|---|---|---|---|---|---|
| | Ionizable | | | | DSPE- | | | | | Lipid to |
| | Lipids | | | DMG | PEG2000- | Lipids | | | | mRNA |
| | Ionizable | Helper | Cholesterol | PEG2000 | Maleimide | Molar Ratio | | | NP | Weight |
| Formulation | lipid (I) | Lipid (H) | (C) | (P1) | (P2) | (I:H:C:P1:P2) | mRNA | Antibody | Ratio | Ratio |
| 1 | Lipid 10 | DSPC | Cholesterol | DMG | DSPE- | 25:38:56.5:2.4:0.3 | EGFP | MX1500 | 9 | 10 |
| | | | | PEG2000 | PEG2000- | | | | | |
| | | | | | Maleimide | | | | | |
| 2 | Lipid 10 | DSPC | Cholesterol | DMG | DSPE- | 25:26:46.5:2.4:0.1 | EGFP | MX1500 | 9 | 10 |
| | | | | PEG2000 | PEG2000- | | | | | |
| | | | | | Maicimide | | | | | |
| 3 | Lipiec 10 | DSPC | Cholesterol | DMG | DSPE- | 25:16:56.5:2.45:0.05 | EGFP | MX1500 | 9 | 10 |
| | | | | PEG2000 | PEG2000- | | | | | |
| | | | | | Maleimide | | | | | |
| 4 | Lipie 10 | DSPC | Cholesterol | DMG | DSPE- | 25:26:46.5:2.45:0.05 | EGFP | MX1500 | 9 | 10 |
| | | | | PEG2000 | PEG2000- | | | | | |
| | | | | | Maleimide | | | | | |

TABLE 27
Biophysical properties of unconjugated and conjugated LNPs with the molar %
of the DSPC at 16% versus 26%, with 0.1% and 0.05% DSPE-PEG2000-Maleimide.
Measurements were taken as described in previous method using ZETASIZER ULTRA ™. LNPs
were diluted 50X in DPBS for size, PI, and charge measurements.

| | | | | | mRNA | mRNA |
|---|---|---|---|---|---|---|
| | | Polydispersity | | | Encapsulation | Recovery |
| | Size | Index | Charge | Particle Conc | Efficiency | Rate |
| LNP | (nm) | (PI) | (mV) | (Particles/ml) | (%) | (%) |
| Lipid10_25_16_0.1 | 69 | 0.09 | −1.31 | 1.39E+13 | 99 | 81 |
| Lipid10_25_16_0.1_MK1500 | 120 | 0.11 | −2.82 | 2.41E+12 | 98 | 87 |
| Lipid10_25_26_0.1 | 76 | 0.08 | −1 | 2.78E+13 | 99 | 86 |
| Lipid10_25_26_0.3_MX1500 | 128 | 0.08 | −2.89 | 2.21E+12 | 98 | 67 |
| Lipid10_25_16_0.05 | 72 | 0.08 | −2.14 | 3.85E+13 | 99 | 85 |
| Lipid10_25_16_0.05_MX1500 | 104 | 0.06 | −4.59 | 6.70E+12 | 98 | 70 |
| Lipid10_25_26_0.05 | 73 | 0.08 | −4.13 | 2.27E+13 | 99 | 79 |
| Lipid10_25_26_0.05_MX1500 | 106 | 0.08 | −2.9 | 2.82E+12 | 98 | 89 |

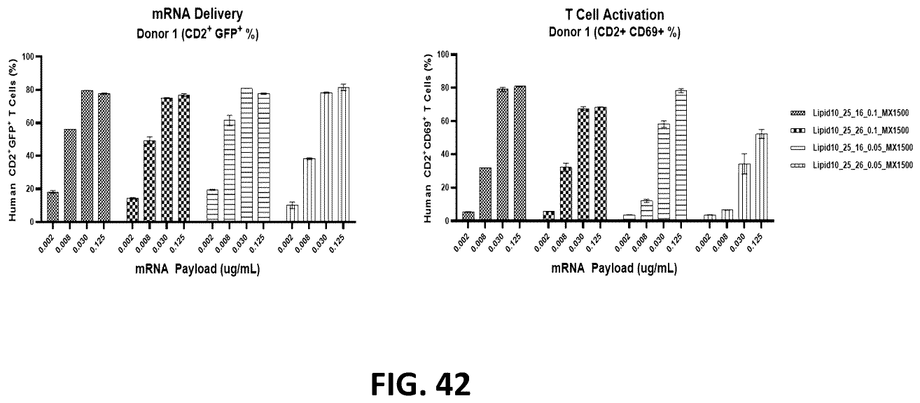

Summary: Increasing the molar % of DSPC in Lipid 10 LNP formulations resulted in reduced in vitro mRNA delivery efficiency to human primary T cells. Increasing the molar % of DSPC in Lipid 10 LNP formulations reduced the T cell activation potential of the conjugated Lipid 10 LNPs in vitro. See FIG. 42.

### Example 21

### LNP Formulation Optimization: N/P Ratio

TABLE 28
In this workflow, the molar % of the DSPC,
Cholesterol, DMG-PEG2000 and DSPE-PEG2000-
Maleimide and Lipid 10 (25%) was constant.

| Name | Molar ratio (%) |
|---|---|
| Cholesterol | 56.5 |
| DSPE-PEG2000-Maleimide | 0.1 |
| DSPC | 16 |
| Lipid 10 | 25 |
| DMG-PEG2000 | 2.4 |

TABLE 29
Formulations tested (N/P ratios of 4.5, 7, and 9).

| | | | | PEG Lipids | | | | | | Ionizable |
|---|---|---|---|---|---|---|---|---|---|---|
| | Ionizable | | | | DSPE- | | | | | Lipid to |
| | Lipids | | | DMG | PEG2000- | Lipids | | | | mRNA |
| | Ionizable | Helper | Cholesterol | PEG2000 | Maleimide | Molar Ratio | | | NP | Weight |
| Formulation | lipid (I) | Lipid (H) | (C) | (P1) | (P2) | (I:H:C:P1:P2) | mRNA | Antibody | Ratio | Ratio |
| 1 | Lipid 10 | DSPC | Cholesterol | DMG | DSPE- | 25:16:56.5:2.4:0.1 | EGFP | MX1500 | 9 | 10 |
| | | | | PEG2000 | PEG2000- | | | | | |
| | | | | | Maleimide | | | | | |
| 2 | Lipid 10 | DSPC | Cholesterol | DMG | DEPE- | 25:16:56.5:2.4:0.1 | EGFP | MX1500 | 7 | 7.78 |
| | | | | PEG2000 | PEG2000- | | | | | |
| | | | | | Maleimide | | | | | |
| 3 | Lipid 10 | DSPC | Cholesterol | DMG | DEPE- | 25:16:56.5:2.4:0.1 | EGFP | MX1500 | 4.5 | 5 |
| | | | | PEG2000 | PEG2000- | | | | | |
| | | | | | Maleimide | | | | | |

TABLE 30
Biophysical properties of unconjugated and conjugated LNPs with the N/P ratio of
4.5, 7, 9. Measurements were taken as described in previous method using ZETASIZER
ULTRA ™. LNPs were diluted 50X in DPBS for size, PI, and charge measurements.

| | | Polydispersity | | mRNA Encapsulation | mRNA Recovery |
|---|---|---|---|---|---|
| LNP | Size (nm) | Index (PI) | Charge (mV) | Efficiency (%) | Rate (%) |
| Lipid10_25_0.1_NP9 | 67 | 0.04 | −2.86 | 97 | 29 |
| Lipid10_25_0.1_NP9_MX1500 | 94 | 0.01 | −2.93 | 94 | 45 |
| Lipid10_25_0.1_NP7 | 69 | 0.04 | −4.5 | 97 | 27 |
| Lipid10_25_0.1_NP7_MX1500 | 94 | 0.03 | −3.05 | 93 | 55 |
| Lipid10_25_0.1_NP4.5 | 63 | 0.04 | −1.83 | 76 | 20 |
| Lipid10_25_0.1_NP4.5_MX1500 | 84 | 0.05 | −2.34 | 89 | 73 |

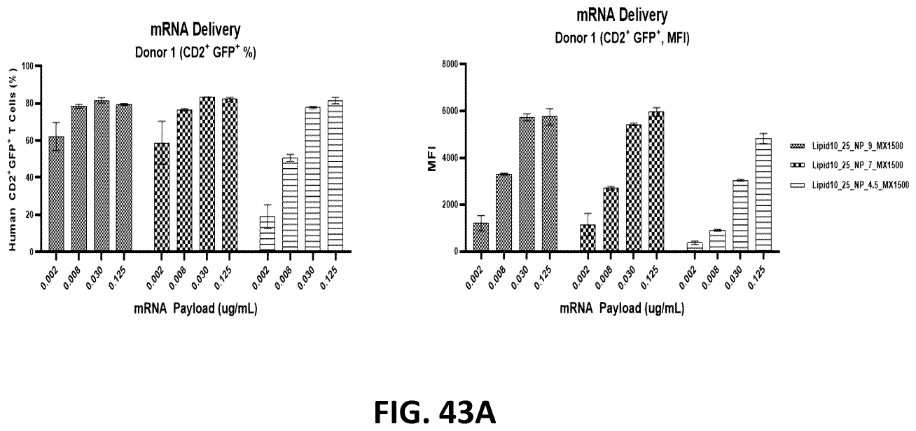

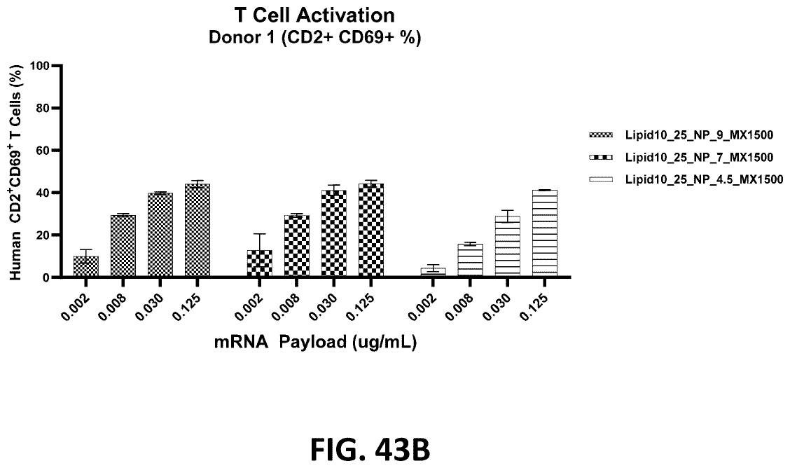

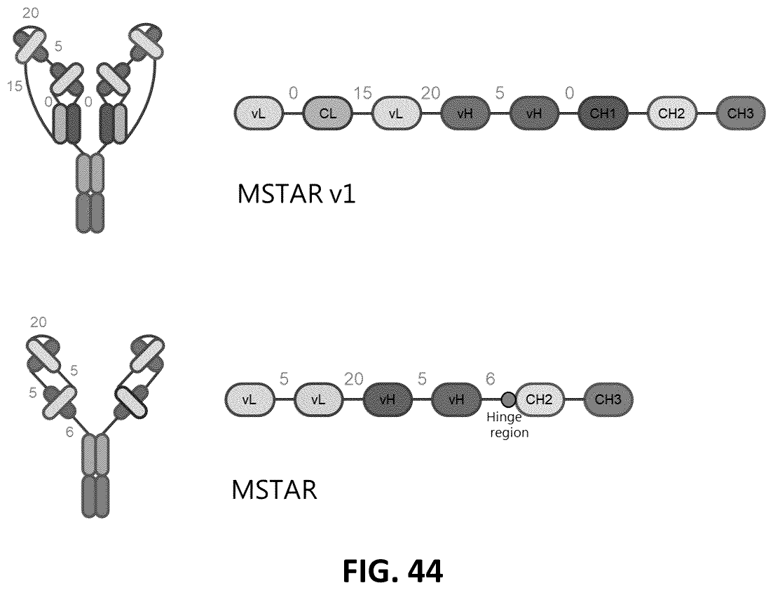

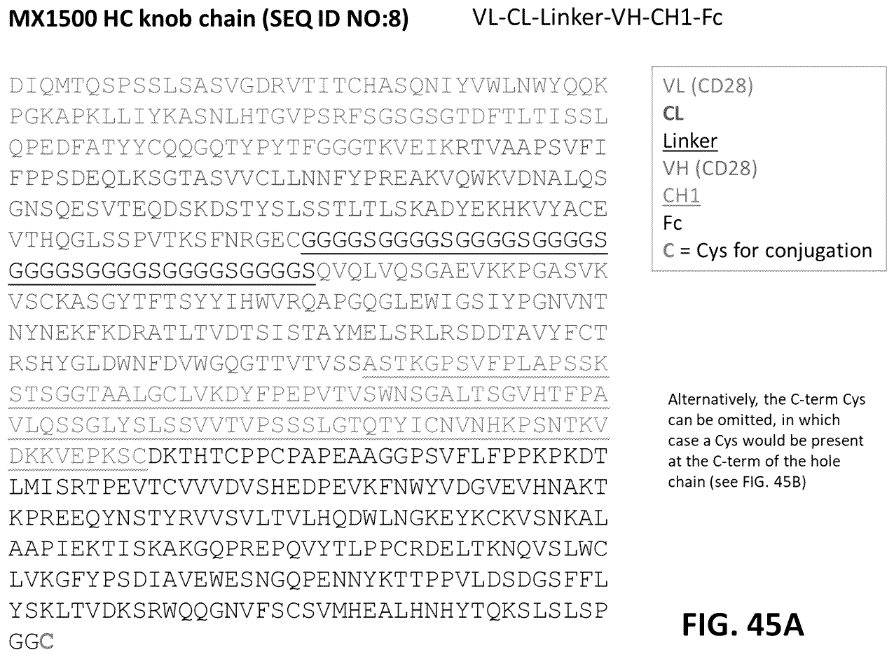

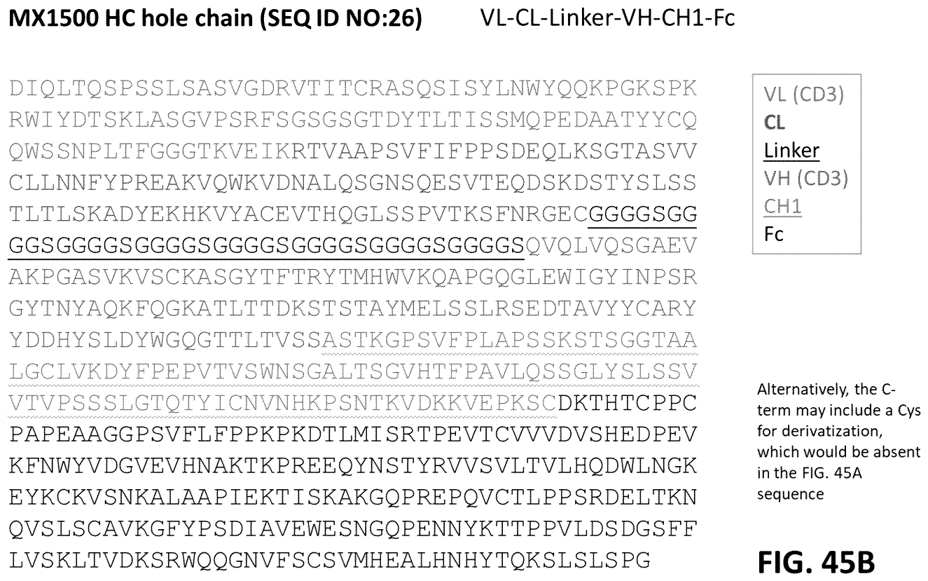

Summary: N/P=9 and N/P=7 showed very similar mRNA delivery potency and T cell activation profiles. N/P=7 can be selected to lower formulation lipid load. N/P=4.5 showed lower potency than N/P=9 or N/P=7 at the lower doses tested. See FIGS. 43A-43B.

TABLE 31
Sequences corresponding to different targeting antibodies that
can be conjugated to the surface of T-cell targeted LNPs.

| | SEQ ID NOS | | |
|---|---|---|---|
| | Heavy Chain | | Light |
| Antibody | Key Chain | Hole Chain | Chain |
| MX1501 | 1 | 19 | |
| MX1505 | 2 | 20 | |
| MX1517 | 3 | 21 | |
| MX1518 | 4 | 22 | |
| MX1507 | 5 | 23 | 37 |
| MX1519 | 6 | 24 | 38 |
| MX1506 | 7 | 25 | 39 |
| MX1500 | 8 | 26 | |
| MX1502 | 9 | 27 | |
| MX1503 | 10 | 28 | |
| MX1504 | 11 | 29 | |
| MX1505 | 12 | 30 | |
| MX1516 | 13 | 31 | |
| MX1520 | 14 | 32 | |
| MX1532 | 15 | 33 | |
| MX1533 | 16 | 34 | |
| MX1542 | 17 | 35 | |
| MX1243 | 18 | 36 | |

### Example 22

### Optimization of UTRs for CAR Expression in Human T Cells In Vitro

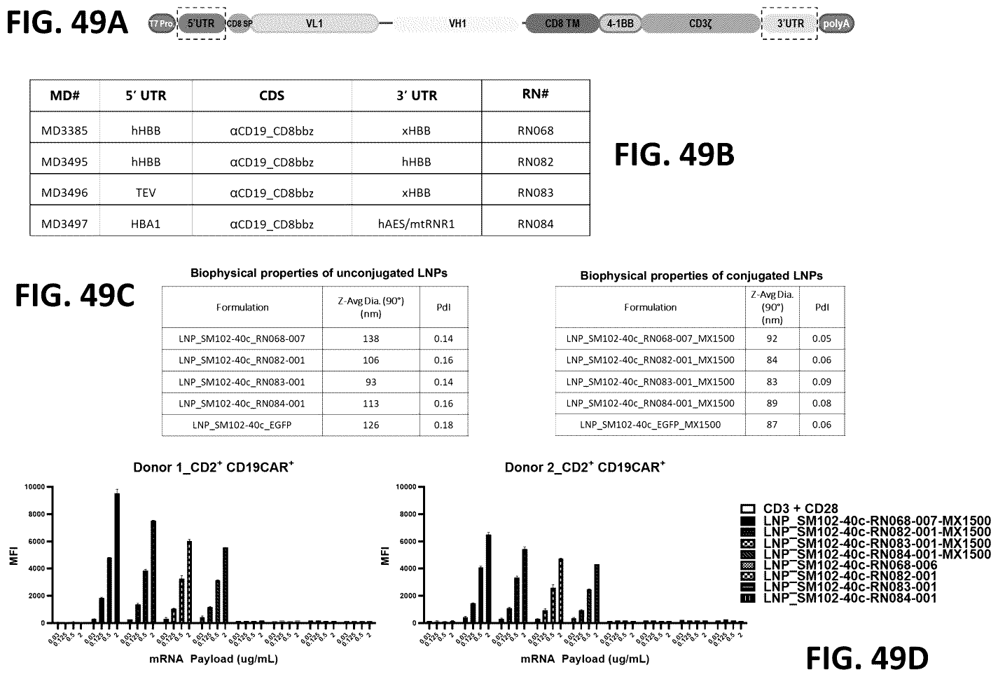

Several combinations of different UTRs were investigated to identify the best combo for human T cell expression delivered by conjugated LNPs. A schematic representation of the constructs used in the optimization process is shown in FIG. 49A. The architecture of the four constructs used in the optimization process, named MD3385, MD3495, MD3496 and MD3497 is shown in FIG. 49B.

DNA plasmids containing the mRNA templates were cloned and produced in NEB Stable E. coli (New England Biolabs). All template sequences contained a T7 promoter, followed by AGA to allow for efficient co-transcriptional capping of the mRNA transcript, a 5′ untranslated region (UTR), the anti-CD19 CAR coding sequence, a 3′UTR, and a 100 nt polyA sequence. The 5′ UTRs were derived from human hemoglobin subunit beta (hHBB), tobacco etch virus (TEV), or human hemoglobin α-globin (HBA1). The 3′ UTRs were derived from hHBB, Xenopus beta-globin (xBG), or a combination of a segment from human mRNA encoding amino-terminal enhancer of split with a segment from mitochondrial 12 S rRNA (AES+mtRNR1).

A minimal UTR construct was also tested, which contained just a 5′ CAAG sequence, and no 3′ UTR. pDNA was linearized by BspQI digestion (New England Biolabs) and purified using QIAquick PCR Purification Kit (Qiagen). mRNAs were produced by IVT using the HiScribe T7 mRNA kit (New England Biolabs), per manufacturer recommendations, with uridine 5′-triphosphate (UTP) replaced by methyl-1 pseudouridine triphosphate (m1ΨTP) (Trilink), and co-transcriptional capping of the transcripts using the trinucleotide capI analog, CleanCap AG (3′ OMe) (TriLink). rNTPs concentrations were adjusted stoichiometrically to match each template, and 50 μg/mL linear pDNA was used. After transcription, DNA was digested with Turbo DNase (Invitrogen).

The synthesized mRNA was isolated by lithium chloride precipitation, with LiCl at a final concentration of 2.5M. The pellet was washed three times with 70% ethanol, dried, resuspended in nuclease-free water, and brought to a concentration of 1 mg/mL. The concentration was measured by nanodrop, and the integrity and purity of mRNA was assessed by capillary electrophoresis on an Agilent 5200 Fragment Analyzer. mRNAs were aliquoted and stored at −80° C.

mRNAs were encapsulated in lipid nanoparticles with NanoAssemblr Ignite microfluidic cartridge technology (Precision Nanosystems). In brief, lipids were dissolved in ethanol at a molar ratio of 40:10:48.5:1.45:0.05 (SM-102 ionizable lipid (Broadpharm):DSPC (Avanti Polar Lipids):cholesterol (Sigma Aldrich):DMG PEG2000: DSPE-PEG2000-Maleimide (Avanti Polar Lipids)), and mRNA was prepared in 100 mM sodium acetate buffer (pH4.5) at 0.1 mg/ml. Then the lipid mixture and mRNA were was prepared using the NanoAssemblr™ Ignite™ system (Cytiva, Wilmington, DE) according to manufacturer's protocol. Briefly, the lipid components were dissolved in ethanol and mRNA reconstituted in 20 mM sodium citrate (pH4.5), followed by mixing of the organic and aqueous solutions at ratio of 1:3 at speed of 10 ml/minute using the Ignite. The prepared LNP was immediately dialyzed against 200× volume of DPBS (pH7.4) twice for 2 hours each time. The LNP was concentrated by centrifugation using 100K Amicon Ultra Centrifuge Filters and sterilized using 0.22 μm syringe filter. Concurrently, Ab decapping was carried out using DTT or TCEP at DTT(TCEP)/Ab molar ratio of 3 at 37° C. for 2 hours followed by using Zaba desalting column to remove the residual DTT/TCEP. Decapped Ab was then mixed with LNP at Ab/maleimide PEG molar ratio of 0.7 and incubate at 25° C. for 2 hours without shaking and at 4° C. overnight. A quenching step using 2:1 (N-Acetyl L-Cysteine/maleimide PEG) molar ratio of N-Acetyl L-cysteine was performed at 25° C. for 1 hours and followed by further purification using CL-4B size exclusion column with DPBS buffer plus 2 mM EDTA. The biophysical properties of the LNP and LNP-Ab were measured using Dynamic Light Scattering (DLS)/Electrophoretic Light Scattering (ELS)(ZETASIZER ULTRA, Malvern, Cambridge,UK), and mRNA content was measured using Ribogreen assay (Thermo Fisher Scientific, Waltham, MA). The biophysical properties of the unconjugated and conjugated LNPs are shown in FIG. 49C.

To transfect unstimulated human PBMCs using conjugated LNPs, LNP-antibody conjugate incubation with PBMC's was set as following: Cryovials containing two Human PBMC donors were taken from cryofreezer and immediately thawed in the 37C water bath. The thawed contents of the cryovials were transferred to 9 mL of pre-warmed RPMI and pelleted by centrifugation at 400×g for 5 minutes at room temperature. The supernatant is removed and replaced with 10 mL pre-warmed RPMI. The PBMC's were pelleted by centrifugation at 400×g for 5 minutes at room temperature. The supernatant was removed and replaced with 10 mL pre-warmed RPMI. The PBMC's were then rested in an incubator at 37C, 5% CO₂ for 2 hours. The PBMC's were then pipetted through a 70 uM strainer (Falcon, Cat. No. 352350) and counted using Trypan blue (Invitrogen, Countess 3 FL). PBMC's are diluted to 6.6×10⁵ cells/mL in warm RPMI and then 100k PBMC's (150 uL) were added to each well of a 96 well round bottom plate. A separate 96-well round bottom plate is used to serially dilute the commercial anti-CD3 (Biolegend, Cat No. B301770) as well as the LNP's. The anti-CD3 and all LNP's were diluted to either 2 nM or 2 ug/mL mRNA payload respectively in 360 μL and serially diluted 4-fold (90 uL+270 uL media) in RPMI (Gibco, Cat. No. 72400-047)+10% FBS (Gibco, 16140-089)+1% Pen/Strep (Gibco, Cat. No. 15140-122). 50 μL of the serially diluted CD3 and/or LNP's were added to their designated wells. 5 uL of 0.1 mg/mL diluted stock of anti-CD28 (Biolegend, Cat. No. B306300) is added to the wells that contain soluble anti-CD3. The plate was incubated at 37° C., 5% CO2 for 20 hours.

Cell Staining for Flow Cytometry Analysis: After the 20-hour incubation time, all well contents were transferred from the Flat bottom 96 well plate to a V-bottom 96 well plate and centrifuged at 400×g for 5 minutes at room temperature. The supernatant was removed, and the cells were washed with 200 uL PBS. The cells were pelleted by centrifugation at 400×g for 5 minutes at room temperature. The supernatant was removed and 100 μL of staining master mix in PBS was added: Viability dye (1/1000, Invitrogen, eFluor 506, Cat. No. 65086618), CD69-PerCP-Cy5.5 (1/100, Biolegend, Cat. No. 310926), CD2-APC/C7 (1/100, Biolegend, Cat. No. 300220), CD4-BV605 (1/100, Biolegend, Cat. No. 317438), CD8-PE (1/100, Biolegend, Cat. No. 301008), and AlexaFluor 647-conjugated anti-CD19CAR idiotypic antibody (10 μg/ml). The plate was incubated for 25 minutes at 4C in the dark. Cells were washed with 150 uL FACS buffer (1×PBS, 1% Bovine Serum Albumin (Fisher, Cat. No. BP1600-1), 0.1% Sodium Azide (Ricca, Cat. No. 7144.8-16). The cells were pelleted by centrifugation at 400×g for 5 minutes at room temperature. The supernatant was removed, the cells were washed with 200 uL FACS buffer and centrifuged at 400×g for 5 minutes at room temperature. The supernatant was removed, and the cells were suspended in 150 uL FACS buffer. Acquisition was performed on an Attune flow cytometer. 125 μL of each sample was acquired with a stop gate set for 5,000 Live CD2+ Cells.

CD19CAR surface expression in human PBMCs was measured as described above. The combo of human β-globin (hBB) 5′ UTR and human β-globin (hBB) 3′ UTR showed the highest level of CD19CAR surface expression. See FIG. 49D.

### Example 23

### Human CD20CAR and Human CD79bCAR Design and Expression in Human PBMCs Delivered by Conjugated LNPs In Vitro

The structure of the CD20 CAR and CD79b CAR used in this example is shown in the FIG. 50A. SM102 based LNPs conjugated to MX1500 were prepared as described previously and used to transfect non-activated human PBMCs (from two donors) in various doses. CD20CAR and CD79bCAR expression were measured 20 hrs post transfection.

Cell surface expression of human CD20CAR from hPBMCs transfected by conjugated and non-conjugated LNPs is shown in FIG. 50B. Surface expression of human CD20CAR was measured using AlexaFluor 647-conjugated anti-CD20CAR idiotypic antibody (10 μg/ml) as previously described flow cytometry method. Cell surface expression of human CD79bCAR from hPBMCs transfected by conjugated and non-conjugated LNPs is shown in FIG. 50C. Surface expression of human CD79bCAR was measured using AlexaFluor 647-conjugated human CD79b protein (20 μg/ml) as previously described flow cytometry method.

TABLE 32
Description of anti-CD20 and anti-CD79b CARs, mRNA encoding the anti-CD20, anti-CD19 and anti-CD79b
CARs used in the T-cell targeted LNPs of the present disclosure and their respective sequences.

| | Construct | | SEQ |
|---|---|---|---|
| Description (molecule type) | name | Protein architecture | ID NO |
| CAR | | | |
| CD20CAR | MX2960, | CD8SP_ahCD20 [ofa]_FcLALPA_CD8bbz | 541 |
| (protein, with signal peptide) | RN105 | | |
| CD20CAR | RN105 | ahCD20 [ofa]_FcLALPA_CD8bbz | 542 |
| (protein, mature, without signal | | | |
| peptide) | | | |
| CD20CAR | MD4056, | ITSv1_5′hHBB_ahCD20 [ofa]_FcLALPA_CD8bbz_3′ | 543 |
| (RNA) | RN105 | | |
| CD79bCAR | MX2961, | CD8SP_anti-hCD79b [23F11wt]_CD8bbz | 544 |
| (protein, with signal peptide) | RN111 | | |
| CD79bCAR | RN111 | anti-hCD79b [23F11wt]_CD8bbz | 545 |
| (protein, mature, without signal | | | |
| peptide) | | | |
| CD79bCAR | MD4143, | mRNA_5′hHBB_CD8SP_anti-hCD79b | 546 |
| (RNA) | RN111 | [23F11wt]_CD8bbz_3′xHBB | |
| UTR_hHBB_xHBB_CD19CAR | RN068 | CD8SP_ahCD19 [hB43]_CD8bbz | 547 |
| (protein, with signal peptide) | | | |
| UTR_hHBB_xHBB_CD19CAR | RN068 | ahCD19 [hB43]_CD8bbz | 548 |
| (protein, mature, without signal | | | |
| peptide) | | | |
| UTR_hHBB_xHBB_CD19CAR | MD3385, | 5′hHBB_CD19_CD8TM_3′xHBB | 549 |
| (RNA) | RN068 | | |
| UTR_hHBB_hHBB_CD19CAR | RN82 | CD8SP_ahCD19 [hB43]_CD8bbz | 550 |
| (protein, with signal peptide) | | | |
| UTR_hHBB_hHBB_CD19CAR | RN82 | ahCD19 [hB43]_CD8bbz | 551 |
| (protein, mature, without signal | | | |
| peptide) | | | |
| UTR_hHBB_hHBB_CD19CAR | MD3495, | 5′hHBB_CD19_CD8TM_3′hHBB | 552 |
| (RNA) | RN082 | | |
| UTR_TEV_xHBB_CD19CAR | RN83 | CD8SP_ahCD19 [hB43]_CD8bbz | 553 |
| (protein, with signal peptide) | | | |
| UTR_TEV_xHBB_CD19CAR | RN83 | ahCD19 [hB43]_CD8bbz | 554 |
| (protein, mature, without signal | | | |
| peptide) | | | |
| UTR_TEV_xHBB_CD19CAR | MD3496, | 5′TEV_CD19_CD8TM_3′xHBB | 555 |
| (RNA) | RN083 | | |
| UTR_hHBA1_hAES/ | RN084 | CD8SP_ahCD19 [hB43]_CD8bbz | 556 |
| mtRNR1_CD19CAR | | | |
| (protein, with signal peptide) | | | |
| UTR_hHBA1_hAES/ | RN084 | ahCD19 [hB43]_CD8bbz | 557 |
| mtRNR1_CD19CAR | | | |
| (protein, mature, without signal | | | |
| peptide) | | | |
| UTR_hHBA1_hAES/ | MD3497, | 5′HBA1_CD19_CD8TM_3′hAES + mtRNR1 | 558 |
| mtRNR1_CD19CAR | RN084 | | |
| (RNA) | | | |

### Example 24

### Active Delivery to Human T Cells Using Anti-CD2 or Anti-CD8 mAb In Vitro

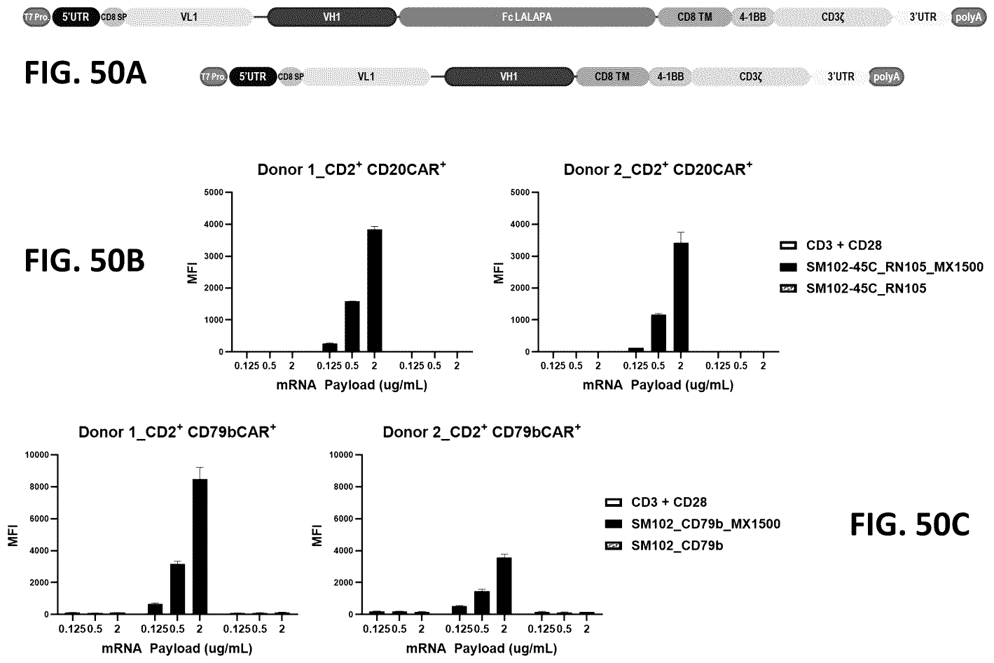

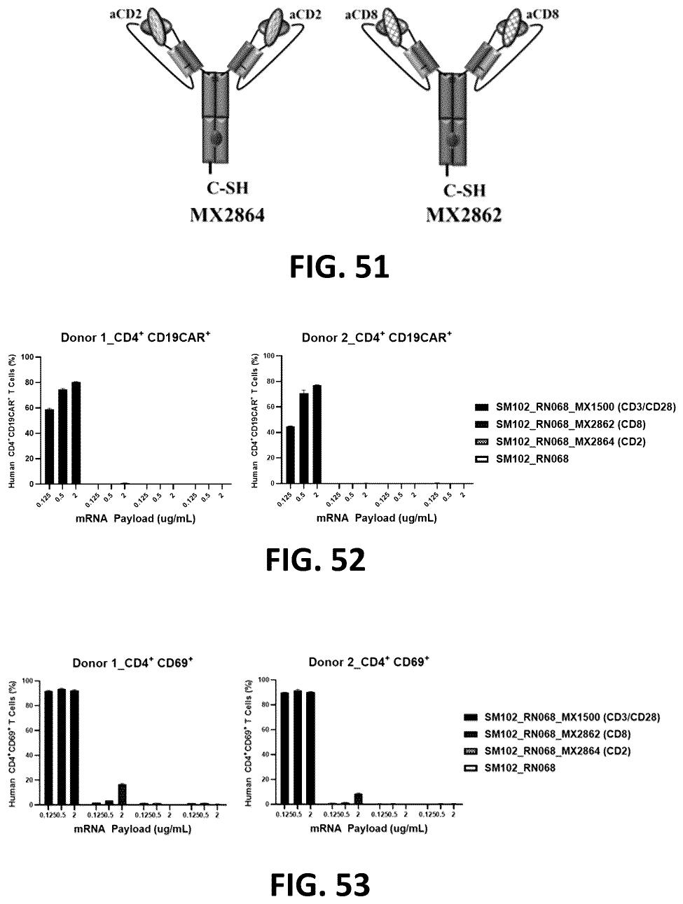

Anti-human CD2 and CD8 monoclonal antibodies were engineered and produced for conjugation as previously described. Diagrams showing the architecture of the anti-CD2 and anti-CD8 monoclonal antibodies are presented in FIG. 51.

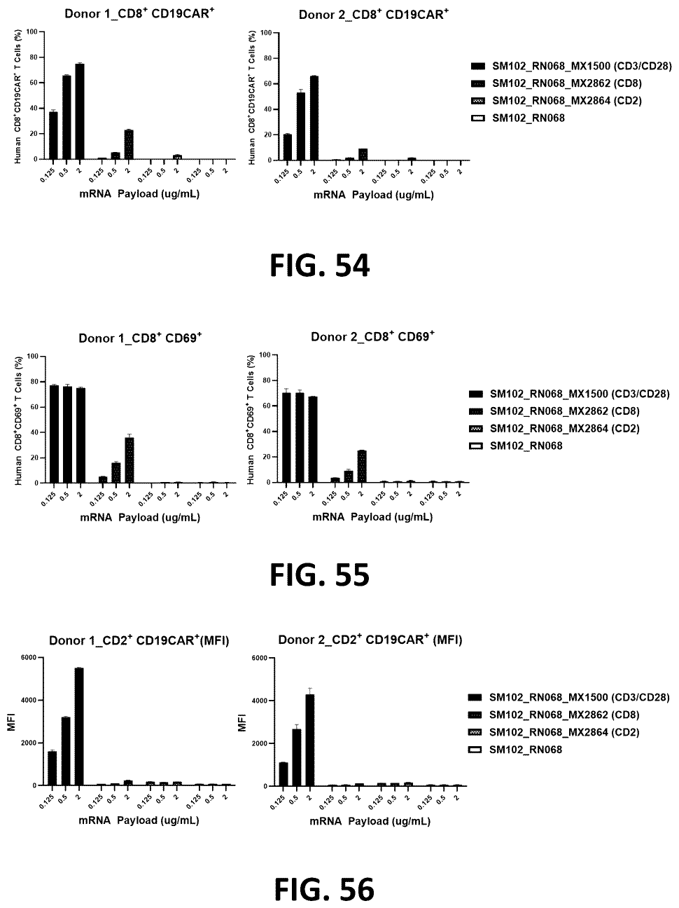

Anti-CD2 or anti-CD8 conjugated to SM102 LNPs with RN068 mRNA cargo were produced as described previously and used to transfect human PBMCs from 2 donors. 20 hours post transfection, cells were collected and CD19CAR surface expression was measured using AlexaFluor 647-conjugated anti-CD19CAR idiotypic antibody (10 μg/ml). CD19CAR expression in CD4 T cells delivered by anti-CD2 conjugated or CD8 conjugated LNPs were minimal while delivery by anti-CD3/CD28 LNP reached a high level. See FIG. 52. The delivery efficiency correlated with the degree of CD4 T cell activation as anti-CD8 showed low but measurable activation of T cells. See FIG. 53. Similarly, anti-CD3/CD28 showed most efficient delivery to human CD8 T cells in vitro, although anti-CD8 demonstrated measurable cargo delivery to CD8 T cells as expected. See FIG. 54. The delivery efficiency correlated with degree of CD8 T cell activation. See FIG. 55. Overall, anti-CD3/CD28 demonstrated significantly higher cargo delivery capacity to human primary T cells in vitro, compared to delivery mediated by anti-CD2 or anti-CD8. See FIG. 56.

TABLE 33
Anti-CD8 and anti-CD2 antibodies, and their respective descriptions and sequence numbers.

| | Construct | | SEQ |
|---|---|---|---|
| Antibody | designation | Construct description | ID NO |
| Anti-hCD8#1 | MX2862 | scFab_ahCD8_OKT8xscFab_ahCD8_OKT8 | 559 |
| (Polypeptide 1) | | hIgGILALAPA-C | |
| Anti-hCD8#1 | MX2862 | | 561 |
| (DNA encoding polypeptide 1) | | | |
| Anti-hCD8#1 | MX2862 | scFab_ahCD8_OKT8xscFab_ahCD8_OKT8 | 560 |
| (Polypeptide 2) | | hIgGILALAPA-C | |
| Anti-hCD8#1 | MX2862 | | 562 |
| (DNA encoding polypeptide 2) | | | |
| Anti-hCD8#2 | MX2863 | scFab_ahCD8_ZED8xscFab_ahCD8_ZED8 | 563 |
| (Polypeptide 1) | | hIgGILALAPA-C | |
| Anti-hCD8#2 | MX2863 | | 565 |
| (DNA encoding polypeptide 1) | | | |
| Anti-hCD8#2 | MX2863 | scFab_ahCD8_ZED8xscFab_ahCD8_ZED8 | 564 |
| (Polypeptide 2) | | hIgGILALAPA-C | |
| Anti-hCD8#2 | MX2863 | | 566 |
| (DNA encoding polypeptide 2) | | | |
| Anti-hCD2 | MX2864 | scFab_ahCD2_sipxscFab_ahCD2_sip | 567 |
| (Polypeptide 1) | | hIgGILALAPA-C | |
| Anti-hCD2 | MX2864 | | 569 |
| (DNA encoding polypeptide 1) | | | |
| Anti-hCD2 | MX2864 | scFab_ahCD2_sipxscFab_ahCD2_sip | 568 |
| (Polypeptide 2) | | hIgGILALAPA-C | |
| Anti-hCD2 | MX2864 | | 570 |
| (DNA encoding polypeptide 2) | | | |

### INCORPORATION BY REFERENCE

The contents of all cited references (including literature references, patents, patent applications, and websites) that may be cited throughout this application are hereby expressly incorporated by reference in their entirety for any purpose, as are the references cited therein, in the versions publicly available on the date the present application was filed Protein and nucleic acid sequences identified by database accession number and other information contained in the subject database entries (e.g., non-sequence related content in database entries corresponding to specific Genbank accession numbers) are incorporated by reference, and correspond to the corresponding database release publicly available on the date the present application was filed.

### EQUIVALENTS

While some aspects have been illustrated and described, the above specification is not restrictive. It will be appreciated that various changes can be made without departing from the spirit and scope of the invention(s). Many variations will become apparent to those skilled in the art upon review of this specification.

It is to be appreciated that the Detailed Description section, and not the Summary and Abstract sections, is intended to be used to interpret the claims. The Summary and Abstract sections may set forth one or more but not all exemplary aspects of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of some aspects will so fully reveal the general nature of the invention that others can, by applying knowledge within the skill of the art, readily modify and/or adapt for various applications such aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

1. A T cell targeted delivery system comprising a lipid nanoparticle (LNP) comprising
(a) a bispecific anti-CD3/anti-CD28 antibody or (b) a monospecific anti-CD3 antibody and a monospecific anti-CD28 antibody anchored to the outer surface of the LNP and a payload encapsulated in the LNP, wherein
(i) the anti-CD3 portion of the bispecific antibody or the monospecific anti-CD3 antibody specifically binds to CD3 on the surface of a T cell; and,
(ii) the anti-CD28 portion of the bispecific antibody or the monospecific anti-CD28 antibody is a costimulator that specifically binds to CD28 on the surface of the T cell; and,
wherein the payload is a chimeric antigen receptor (CAR).

2. (canceled)

3. The T cell targeted delivery system of claim 1, wherein the bispecific anti-CD3/anti-CD28 antibody comprises two polypeptides, each having a structure according to the formula VL-CL-Linker-VH-CH1-Fc, wherein VL is a light chain variable region, CL is a light chain constant region, VH is a heavy chain variable regions, CH1 is a heavy chain constant domain 1, and Fc is an Fc domain.

4. The T cell targeted delivery system of claim 1, further comprising an anti-CD2 antibody and/or an anti-CD8 antibody covalently attached to the outer surface of the LNP.

5. The T cell targeted delivery system of claim 1, wherein the bispecific anti-CD3/anti-CD28 antibody or monospecific anti-CD3 and anti-CD28 antibodies anchored to the outer surface of the LNP are covalently attached to a lipid via maleimide chemistry.

6. The T cell targeted delivery system of claim 1, wherein
(i) the bispecific anti-CD3/anti-CD28 antibody is MX1500 (SEQ ID NOS: 8 and 26);
(ii) the bispecific anti-CD3/anti-CD28 antibody is MX1243 (SEQ ID NOS: 18 and 36): or,
(iii) the monospecific anti-CD3 antibody is MX1507 (SEQ ID NOS: 5, 23, and 37) and the monospecific anti-CD28 antibody is MX1506 (SEQ ID NOS: 7, 25, and 39).

7-9. (canceled)

10. The T cell targeted delivery system of claim 4, wherein the anti-CD2 antibody is MX2864 (SEQ ID NOS: 567 and 568) and/or the anti-CD8 antibody is MX2862 (SEQ ID NOS:
559 and 560) or MX2863 (SEQ ID NOS: 563 and 564).

11. The T cell targeted delivery system of any one of claims 1 to 10, wherein the LNP comprises an ionizable cationic lipid (iLipid) selected from the group consisting of cKK-E12, MC3, SM-102, ACL-0315, KC2, Lipid A6, Lipid M, Lipid 10, C14-4, and any one of MDX1-MDX13.

12. (canceled)

13. The T cell targeted delivery system of claim 1, comprising 27.5 mol % of iLipid; 16 mol % of DSPC; 2.45 mol % of DMG-PEG2000; 0.05 mol % of DSPE-PEG2000-maleimide; and 54 mol % of cholesterol.

14. The T-cell target delivery system of claim 1, wherein the CAR is a CD20-specific CAR, a CD79b-specific CAR, or a CD19-specific CAR.

15. The T-cell target delivery system of claim 14, wherein the CD20-specific CAR is RN105 (SEQ ID NO: 542), the CD79b-specific CAR is RN111 (SEQ ID NO: 545), and the CD19-specific CAR is RN068 (SEQ ID NO: 548), RN082 (SEQ ID NO: 551), RN083 (SEQ ID NO: 554), or RN084 (SEQ ID NO: 557).

16-29. (canceled)

30. A T cell targeted delivery system comprising an LNP comprising a bispecific anti-CD3/anti-CD28 antibody anchored to the outer surface of the LNP and a payload encapsulated in the LNP, wherein
(i) the anti-CD3 portion of the bispecific antibody specifically binds to CD3 on the surface of a T cell; and,
(ii) the anti-CD28 portion of the bispecific antibody is a costimulator that specifically binds to CD28 on the surface of the T cell;
wherein the payload is a CAR selected from the group consisting of a CD19-specific CAR, a CD20-specific CAR, and a CD79b-specific CAR; and,
wherein the bispecific anti-CD3/anti-CD28 antibody comprises two polypeptides, each having a structure according to the formula VL-CL-Linker-VH-CH1-Fc, wherein VL is a light chain variable region, CL is a light chain constant region, VH is a heavy chain variable regions, CH1 is a heavy chain constant domain 1, and Fc is an Fc domain.

31. A pharmaceutical composition comprising a T-cell target delivery system of claim 1 and a pharmaceutically acceptable excipient.

32. A method to treat a disease or condition comprising administering the T-cell targeted delivery system of claim 1 to a subject in need thereof.

33. The method of claim 32, wherein the disease or condition is cancer.

34. An iLipid selected from the group consisting of

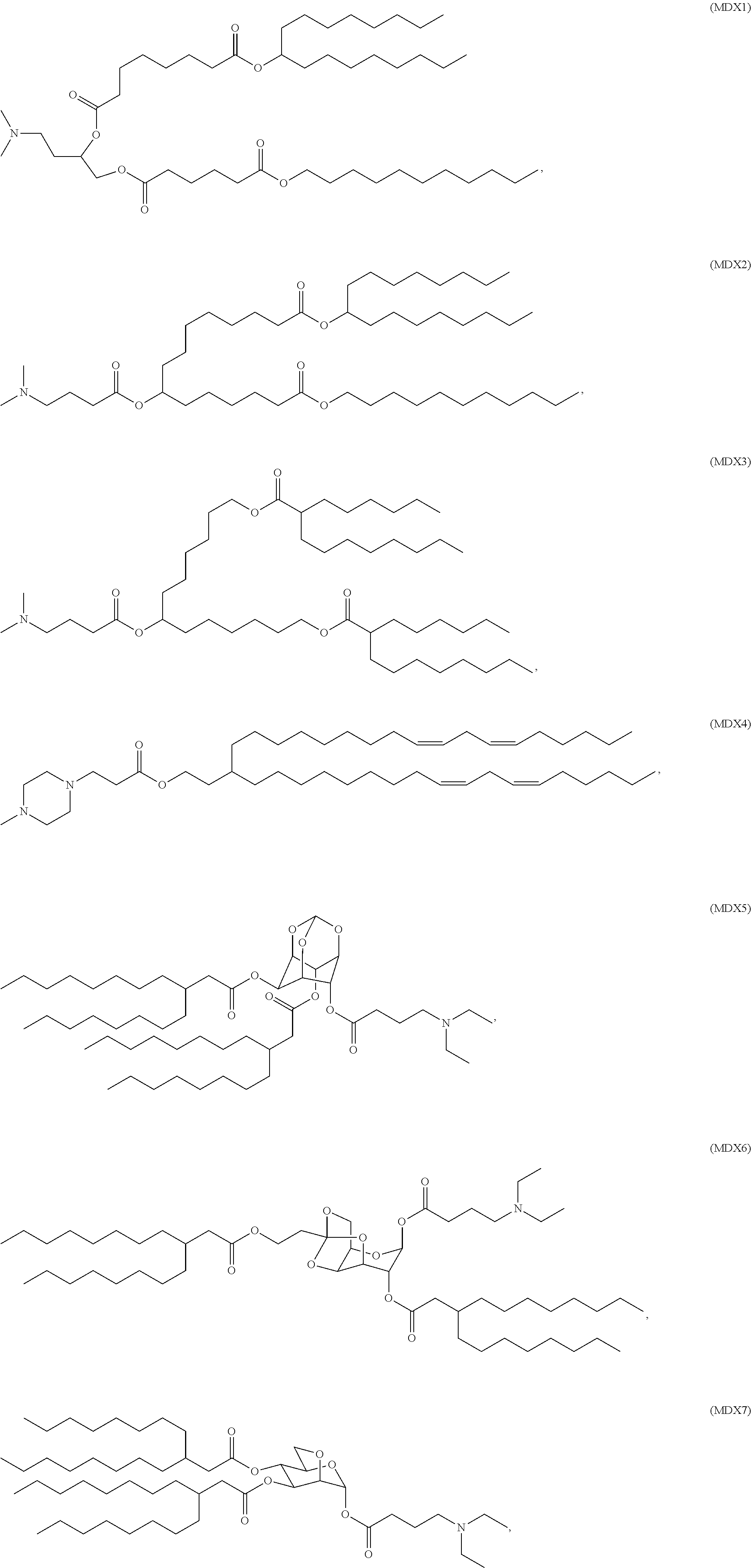

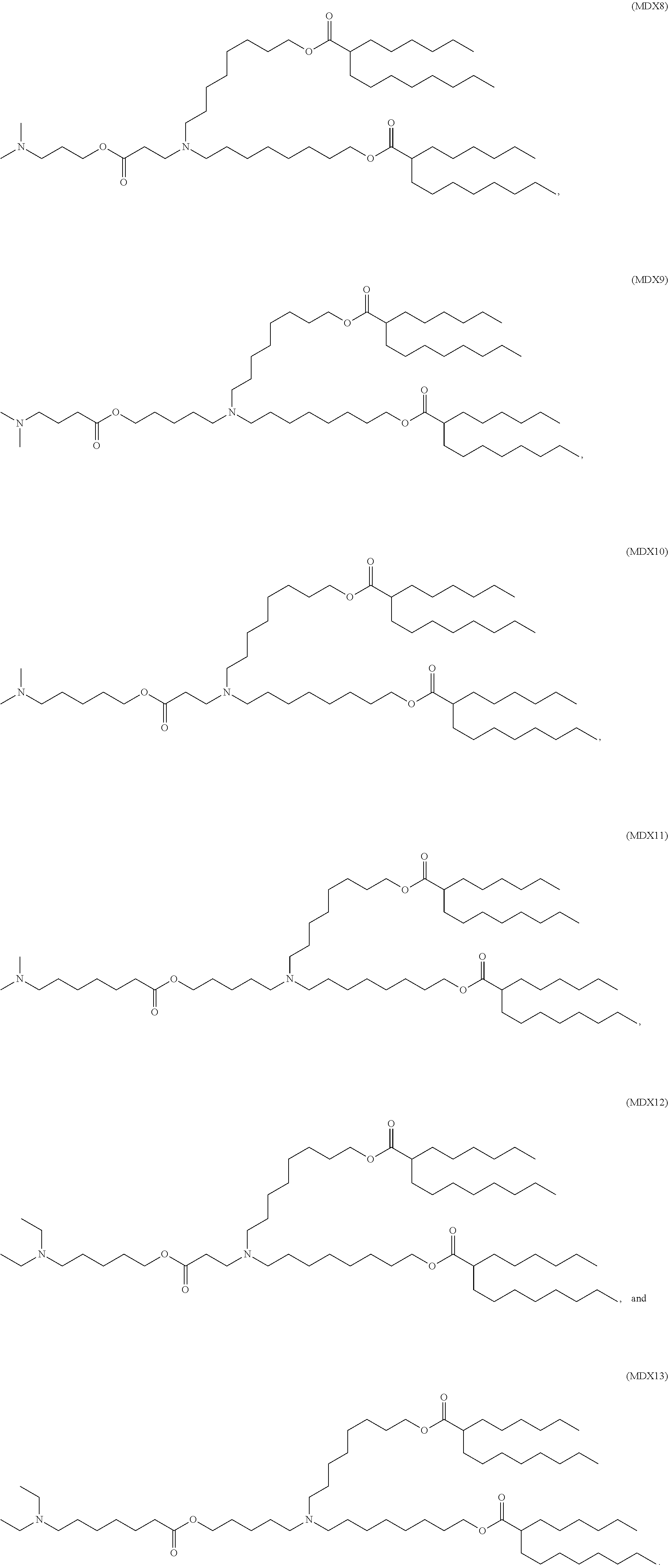

35. An LNP comprising an iLipid of claim 34.

36. A T cell targeted delivery system comprising an iLipid of claim 34.

37. A pharmaceutical composition comprising (i) an LNP or T cell delivery system comprising the iLipid of claim 34, and (ii) a pharmaceutically acceptable excipient.

38. A method to treat a disease or condition comprising administering an LNP, T cell delivery system, or pharmaceutical composition comprising an iLipid of claim 34 to a subject in need thereof.

39. The method of claim 38, wherein the disease or condition is cancer.


SEQUENCE LISTING (570 sequences)

SEQ ID NO: 1 - protein, 940 aa, synthetic construct
  source 1..940: note = MX1501 HC knob chain
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGSSGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGSSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGSGSGGGGSASGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 2 - protein, 930 aa, synthetic construct
  source 1..930: note = MX1505 HC knob chain
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGSSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 3 - protein, 948 aa, synthetic construct
  source 1..948: note = MX1517 HC knob chain
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGSSGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGSSGQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGSGSGGGGSASGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 4 - protein, 948 aa, synthetic construct
  source 1..948: note = MX1518 HC knob chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGGGSGGSGSGGGGSASGSGQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGSGSGGGGSASGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 5 - protein, 450 aa, synthetic construct
  source 1..450: note = MX1507 HC knob chain
QVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 6 - protein, 452 aa, synthetic construct
  source 1..452: note = MX1519 HC knob chain
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 7 - protein, 451 aa, synthetic construct
  source 1..451: note = MX1506 HC knob chain
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 8 - protein, 705 aa, synthetic construct
  source 1..705: note = MX1500 HC knob chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 9 - protein, 940 aa, synthetic construct
  source 1..940: note = MX1502 HC knob chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGSGSGGGGSASGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 10 - protein, 705 aa, synthetic construct; identical to SEQ ID NO: 8
  source 1..705: note = MX1503 HC knob chain

SEQ ID NO: 11 - protein, 705 aa, synthetic construct; identical to SEQ ID NO: 8
  source 1..705: note = MX1504 HC knob chain

SEQ ID NO: 12 - protein, 930 aa, synthetic construct; identical to SEQ ID NO: 2
  source 1..930: note = MX1505 HC knob chain

SEQ ID NO: 13 - protein, 705 aa, synthetic construct; identical to SEQ ID NO: 8
  source 1..705: note = MX1516 HC knob chain

SEQ ID NO: 14 - protein, 481 aa, synthetic construct
  source 1..481: note = MX1520 HC knob chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 15 - protein, 703 aa, synthetic construct
  source 1..703: note = MX1532 HC knob chain
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 16 - protein, 703 aa, synthetic construct; identical to SEQ ID NO: 15
  source 1..703: note = MX1533 HC knob chain

SEQ ID NO: 17 - protein, 711 aa, synthetic construct
  source 1..711: note = MX1542 HC knob chain
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 18 - protein, 481 aa, synthetic construct; identical to SEQ ID NO: 14
  source 1..481: note = MX1243 HC knob chain

SEQ ID NO: 19 - protein, 232 aa, synthetic construct
  source 1..232: note = MX1501 HC hole chain
ASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 20 - protein, 232 aa, synthetic construct; identical to SEQ ID NO: 19
  source 1..232: note = MX1505 HC hole chain

SEQ ID NO: 21 - protein, 232 aa, synthetic construct; identical to SEQ ID NO: 19
  source 1..232: note = MX1517 HC hole chain

SEQ ID NO: 22 - protein, 232 aa, synthetic construct; identical to SEQ ID NO: 19
  source 1..232: note = MX1518 HC hole chain

SEQ ID NO: 23 - protein, 448 aa, synthetic construct
  source 1..448: note = MX1507 HC hole chain
QVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 24 - protein, 450 aa, synthetic construct
  source 1..450: note = MX1519 HC hole chain
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 25 - protein, 449 aa, synthetic construct
  source 1..449: note = MX1506 HC hole chain
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 26 - protein, 701 aa, synthetic construct
  source 1..701: note = MX1500 HC hole chain
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 27 - protein, 232 aa, synthetic construct; identical to SEQ ID NO: 19
  source 1..232: note = MX1502 HC hole chain

SEQ ID NO: 28 - protein, 703 aa, synthetic construct
  source 1..703: note = MX1503 HC hole chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 29 - protein, 232 aa, synthetic construct; identical to SEQ ID NO: 19
  source 1..232: note = MX1504 HC hole chain

SEQ ID NO: 30 - protein, 232 aa, synthetic construct; identical to SEQ ID NO: 19
  source 1..232: note = MX1505 HC hole chain

SEQ ID NO: 31 - protein, 709 aa, synthetic construct
  source 1..709: note = MX1516 HC hole chain
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 32 - protein, 485 aa, synthetic construct
  source 1..485: note = MX1520 HC hole chain
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGGGSGGSGSGGGGSASGSGQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 33 - protein, 701 aa, synthetic construct; identical to SEQ ID NO: 26
  source 1..701: note = MX1532 HC hole chain

SEQ ID NO: 34 - protein, 232 aa, synthetic construct; identical to SEQ ID NO: 19
  source 1..232: note = MX1533 HC hole chain

SEQ ID NO: 35 - protein, 709 aa, synthetic construct; identical to SEQ ID NO: 31
  source 1..709: note = MX1542 HC hole chain

SEQ ID NO: 36 - protein, 477 aa, synthetic construct
  source 1..477: note = MX1243HC hole chain
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 37 - protein, 213 aa, synthetic construct
  source 1..213: note = MX1507 LC
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 219 aa, synthetic construct
  source 1..219: note = MX1519 LC
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 214 aa, synthetic construct
  source 1..214: note = MX1506 LC
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 119 aa, synthetic construct
  source 1..119: note = aCD3_vH
QVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSS

SEQ ID NO: 41 - protein, 106 aa, synthetic construct
  source 1..106: note = aCD3_vL
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIK

SEQ ID NO: 42 - protein, 120 aa, synthetic construct
  source 1..120: note = aCD28_vH
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSS

SEQ ID NO: 43 - protein, 107 aa, synthetic construct
  source 1..107: note = aCD28_vL
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 44 - protein, 121 aa, synthetic construct
  source 1..121: note = SARCD3_vH
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 45 - protein, 112 aa, synthetic construct
  source 1..112: note = SARCD3_vL
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 46 - protein, 5 aa, synthetic construct
  source 1..5: note = aCD3_vH_CDR1
RYTMH

SEQ ID NO: 47 - protein, 17 aa, synthetic construct
  source 1..17: note = aCD3_vH_CDR2
YINPSRGYTNYAQKFQG

SEQ ID NO: 48 - protein, 10 aa, synthetic construct
  source 1..10: note = aCD3_vH_CDR3
YYDDHYSLDY

SEQ ID NO: 49 - protein, 10 aa, synthetic construct
  source 1..10: note = aCD3_vL_CDR1
RASQSISYLN

SEQ ID NO: 50 - protein, 7 aa, synthetic construct
  source 1..7: note = aCD3_vL_CDR2
DTSKLAS

SEQ ID NO: 51 - protein, 10 aa, synthetic construct
  source 1..10: note = aCD3_vL_CDR3
QQWSSNPLTF

SEQ ID NO: 52 - protein, 5 aa, synthetic construct
  source 1..5: note = aCD28_vH_CDR1
SYYIH

SEQ ID NO: 53 - protein, 17 aa, synthetic construct
  source 1..17: note = aCD28_vH_CDR2
SIYPGNVNTNYNEKFKD

SEQ ID NO: 54 - protein, 11 aa, synthetic construct
  source 1..11: note = aCD28_vH_CDR3
SHYGLDWNFDV

SEQ ID NO: 55 - protein, 10 aa, synthetic construct
  source 1..10: note = aCD28_vL_CDR1
HASQNIYVWL

SEQ ID NO: 56 - protein, 7 aa, synthetic construct
  source 1..7: note = aCD28_vL_CDR2
KASNLHT

SEQ ID NO: 57 - protein, 9 aa, synthetic construct
  source 1..9: note = aCD28_vL_CDR3
QQGQTYPYT

SEQ ID NO: 58 - protein, 5 aa, synthetic construct
  source 1..5: note = SARCD3_vH_CDR1
KAWMH

SEQ ID NO: 59 - protein, 19 aa, synthetic construct
  source 1..19: note = SARCD3_vH_CDR2
QIKDKSNSYATYYADSVKG

SEQ ID NO: 60 - protein, 10 aa, synthetic construct
  source 1..10: note = SARCD3_vH_CDR3
VYYALSPFDY

SEQ ID NO: 61 - protein, 16 aa, synthetic construct
  source 1..16: note = SARCD3_vL_CDR1
KSSQSLVHNNANTYLS

SEQ ID NO: 62 - protein, 7 aa, synthetic construct
  source 1..7: note = SARCD3_vL_CDR2
KVSNRFS

SEQ ID NO: 63 - protein, 8 aa, synthetic construct
  source 1..8: note = SARCD3_vL_CDR3
GQGTQYPF

SEQ ID NO: 64 - DNA, 2820 bp, synthetic construct
  source 1..2820: mol_type = other DNA; note = MD2094 HC knob
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggcggatcctctggagacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggaggtggtggctctggcggatctgggagtggtggcggaggctctgcttctggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctggaggcagctccgggcaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggcggaggatctggtggatccggatctggcggaggcggatctgcatcaggatccggagcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 65 - DNA, 2790 bp, synthetic construct
  source 1..2790: mol_type = other DNA; note = MD2176 HC knob
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggcggaggatctggcggaggtggaagtggcggaggcggatccgacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggaggtggtggctctggcggatctgggagtggtggcggaggctctgcttctggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctggaggcagctccgggcaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 66 - DNA, 2844 bp, synthetic construct
  source 1..2844: mol_type = other DNA; note = MD2136 HC knob
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagggcggatcctctggagacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggaggtggtggctctggcggatctgggagtggtggcggaggctctgcttctggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctggaggcagctccgggcaagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggcggaggatctggtggatccggatctggcggaggcggatctgcatcaggatccggagcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 67 - DNA, 2844 bp, synthetic construct
  source 1..2844: mol_type = other DNA; note = MD2194 HC knob
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggcggatcctctggagacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagggaggtggtggctctggcggatctgggagtggtggcggaggctctgcttctggatctggacaagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctggaggcagctccgggcaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggcggaggatctggtggatccggatctggcggaggcggatctgcatcaggatccggagcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 68 - DNA, 1350 bp, synthetic construct
  source 1..1350: mol_type = other DNA; note = MD2127 HC knob
caggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 69 - DNA, 1356 bp, synthetic construct
  source 1..1356: mol_type = other DNA; note = MD2130 HC knob
caagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 70 - DNA, 1353 bp, synthetic construct
  source 1..1353: mol_type = other DNA; note = MD2133 HC knob
caggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 71 - DNA, 2116 bp, synthetic construct
  source 1..2116: mol_type = other DNA; note = MD2155 HC knob
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgtt

SEQ ID NO: 72 - DNA, 2820 bp, synthetic construct
  source 1..2820: mol_type = other DNA; note = MD2154 HC knob
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggcggatcctctggagacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggaggtggtggctctggcggatctgggagtggtggcggaggctctgcttctggatctggacaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctggaggcagctccgggcaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggcggaggatctggtggatccggatctggcggaggcggatctgcatcaggatccggagcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 73 - DNA, 2116 bp, synthetic construct; identical to SEQ ID NO: 71
  source 1..2116: mol_type = other DNA; note = MD2155 HC knob

SEQ ID NO: 74 - DNA, 2116 bp, synthetic construct; identical to SEQ ID NO: 71
  source 1..2116: mol_type = other DNA; note = MD2155 HC knob

SEQ ID NO: 75 - DNA, 2790 bp, synthetic construct; identical to SEQ ID NO: 65
  source 1..2790: mol_type = other DNA; note = MD2176 HC knob

SEQ ID NO: 76 - DNA, 2116 bp, synthetic construct; identical to SEQ ID NO: 71
  source 1..2116: mol_type = other DNA; note = MD2155 HC knob

SEQ ID NO: 77 - DNA, 1444 bp, synthetic construct
  source 1..1444: mol_type = other DNA; note = MD2138 HC knob
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggaggtggtggctctggcggatctgggagtggtggcggaggctctgcttctggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgtt

SEQ ID NO: 78 - DNA, 2109 bp, synthetic construct
  source 1..2109: mol_type = other DNA; note = MD2177 HC knob
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 79 - DNA, 2109 bp, synthetic construct; identical to SEQ ID NO: 78
  source 1..2109: mol_type = other DNA; note = MD2177 HC knob

SEQ ID NO: 80 - DNA, 2133 bp, synthetic construct
  source 1..2133: mol_type = other DNA; note = MD2096 HC knob
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 81 - DNA, 1443 bp, synthetic construct
  source 1..1443: mol_type = other DNA; note = MD2138 HC knob
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggaggtggtggctctggcggatctgggagtggtggcggaggctctgcttctggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggctgt

SEQ ID NO: 82 - DNA, 696 bp, synthetic construct
  source 1..696: mol_type = other DNA; note = MD2174_stump HC hole
gcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 83 - DNA, 696 bp, synthetic construct; identical to SEQ ID NO: 82
  source 1..696: mol_type = other DNA; note = MD2174_stump HC hole

SEQ ID NO: 84 - DNA, 696 bp, synthetic construct; identical to SEQ ID NO: 82
  source 1..696: mol_type = other DNA; note = MD2174_stump HC hole

SEQ ID NO: 85 - DNA, 696 bp, synthetic construct; identical to SEQ ID NO: 82
  source 1..696: mol_type = other DNA; note = MD2174_stump HC hole

SEQ ID NO: 86 - DNA, 1344 bp, synthetic construct
  source 1..1344: mol_type = other DNA; note = MD2128 HC hole
caggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 87 - DNA, 1350 bp, synthetic construct
  source 1..1350: mol_type = other DNA; note = MD2131 HC hole
caagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 88 - DNA, 1347 bp, synthetic construct
  source 1..1347: mol_type = other DNA; note = MD2134 HC hole
caggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 89 - DNA, 2103 bp, synthetic construct
  source 1..2103: mol_type = other DNA; note = MD2175 HC hole
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 90 - DNA, 696 bp, synthetic construct; identical to SEQ ID NO: 82
  source 1..696: mol_type = other DNA; note = MD2174_stump HC hole

SEQ ID NO: 91 - DNA, 2112 bp, synthetic construct
  source 1..2112: mol_type = other DNA; note = MD2095 HC hole
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctggatag

SEQ ID NO: 92 - DNA, 696 bp, synthetic construct; identical to SEQ ID NO: 82
  source 1..696: mol_type = other DNA; note = MD2172 stump HC hole

SEQ ID NO: 93 - DNA, 696 bp, synthetic construct; identical to SEQ ID NO: 82
  source 1..696: mol_type = other DNA; note = MD2174_stump HC hole

SEQ ID NO: 94 - DNA, 2127 bp, synthetic construct
  source 1..2127: mol_type = other DNA; note = MD2097 HC hole
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 95 - DNA, 1455 bp, synthetic construct
  source 1..1455: mol_type = other DNA; note = MD2153 HC hole
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagggaggtggtggctctggcggatctgggagtggtggcggaggctctgcttctggatctggacaagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 96 - DNA, 2103 bp, synthetic construct; identical to SEQ ID NO: 89
  source 1..2103: mol_type = other DNA; note = MD2175 HC hole

SEQ ID NO: 97 - DNA, 696 bp, synthetic construct; identical to SEQ ID NO: 82
  source 1..696: mol_type = other DNA; note = MD2174_stump HC hole

SEQ ID NO: 98 - DNA, 2127 bp, synthetic construct; identical to SEQ ID NO: 94
  source 1..2127: mol_type = other DNA; note = MD2097 HC hole

SEQ ID NO: 99 - DNA, 1431 bp, synthetic construct
  source 1..1431: mol_type = other DNA; note = MD2139 HC hole
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggaggtggtggctctggcggatctgggagtggtggcggaggctctgcttctggatctggacaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 100 - DNA, 639 bp, synthetic construct
  source 1..639: mol_type = other DNA; note = DNA MD2129 LC
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 101 - DNA, 657 bp, synthetic construct
  source 1..657: mol_type = other DNA; note = DNA MD2132 LC
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 102 - DNA, 642 bp, synthetic construct
  source 1..642: mol_type = other DNA; note = DNA MD2135 LC
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 103 - DNA, 357 bp, synthetic construct
  source 1..357: mol_type = other DNA; note = aCD3_vH
caggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatct

SEQ ID NO: 104 - DNA, 318 bp, synthetic construct
  source 1..318: mol_type = other DNA; note = aCD3_vL
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaag

SEQ ID NO: 105 - DNA, 360 bp, synthetic construct
  source 1..360: mol_type = other DNA; note = aCD28_vH
caggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttct

SEQ ID NO: 106 - DNA, 321 bp, synthetic construct
  source 1..321: mol_type = other DNA; note = aCD28_vL
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaag

SEQ ID NO: 107 - DNA, 363 bp, synthetic construct
  source 1..363: mol_type = other DNA; note = SARCD3_vH
caagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatct

SEQ ID NO: 108 - DNA, 336 bp, synthetic construct
  source 1..336: mol_type = other DNA; note = SARCD3_vL
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaag

SEQ ID NO: 109 - protein, 4 aa, synthetic construct
  source 1..4: note = Linker
GGSG

SEQ ID NO: 110 - protein, 5 aa, synthetic construct
  source 1..5: note = Linker
GSSGS

SEQ ID NO: 111 - protein, 5 aa, synthetic construct
  source 1..5: note = Linker
GTVAA

SEQ ID NO: 112 - protein, 5 aa, synthetic construct
  source 1..5: note = Linker
ASGGS

SEQ ID NO: 113 - protein, 5 aa, synthetic construct
  source 1..5: note = Linker
ASTGG

SEQ ID NO: 114 - protein, 6 aa, synthetic construct
  source 1..6: note = Linker
ASGGSG

SEQ ID NO: 115 - protein, 10 aa, synthetic construct
  source 1..10: note = Linker
GGSGGSSGSS

SEQ ID NO: 116 - protein, 10 aa, synthetic construct
  source 1..10: note = Linker
SGGSGSSGGS

SEQ ID NO: 117 - protein, 17 aa, synthetic construct
  source 1..17: note = Linker
GGSGGSGSGGGSASGSG

SEQ ID NO: 118 - protein, 18 aa, synthetic construct
  source 1..18: note = Linker
GGSGGSGSGGGGSASGSG

SEQ ID NO: 119 - protein, 20 aa, synthetic construct
  source 1..20: note = Linker
GGGSSGGGGSGGSGSGGSGS

SEQ ID NO: 120 - protein, 20 aa, synthetic construct
  source 1..20: note = Linker
GGGGSGGSGSGGGGSASGSG

SEQ ID NO: 121 - protein, 20 aa, synthetic construct
  source 1..20: note = Linker
GGGSSGGSGSGGSGSGGSGS

SEQ ID NO: 122 - protein, 22 aa, synthetic construct
  source 1..22: note = Linker
SGGSSGGSGSGGSGSGGSGSSG

SEQ ID NO: 123 - protein, 22 aa, synthetic construct
  source 1..22: note = Linker
GSGSSGGGGSGGSGSGGSGSSG

SEQ ID NO: 124 - protein, 40 aa, synthetic construct
  source 1..40: note = Linker
GGGGSGSGGSGGGSSGGGGSGGGGSGGGGSGGGGSGGGGS

SEQ ID NO: 125 - protein, 40 aa, synthetic construct
  source 1..40: note = Linker
GGGGSGGGGSGGGGSGGGGSGGGGSGGGGSGGGGSGGGGS

SEQ ID NO: 126 - protein, 43 aa, synthetic construct
  source 1..43: note = Linker
GGGGSGSGGSGGGSSGGGGSGGGGSGGGGSGGGGSGGGGSSSS

SEQ ID NO: 127 - protein, 45 aa, synthetic construct
  source 1..45: note = Linker
GGGGSGSGGSGGGSSGGGGSGGGGSGGGGSGGGGSGGGGSSSSGS

SEQ ID NO: 128 - protein, 5 aa, synthetic construct
  source 1..5: note = Linker
GGSGG

SEQ ID NO: 129 - protein, 20 aa, synthetic construct
  source 1..20: note = Linker
GSGGSAGSGSGGGGSASGSG

SEQ ID NO: 130 - protein, 5 aa, synthetic construct
  source 1..5: note = Linker
GGGGS

SEQ ID NO: 131 - protein, 20 aa, synthetic construct
  source 1..20: note = Linker
GSGGSGGSGSGGGGSASGSG

SEQ ID NO: 132 - protein, 107 aa, synthetic construct [sequence omitted]

SEQ ID NO: 133 - protein, 103 aa, synthetic construct
  source 1..103: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 134 - protein, 226 aa, synthetic construct
  source 1..226: note = Fc (LALAPAhole Fc)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 135 - protein, 228 aa, synthetic construct
  source 1..228: note = Fc (LALAPAhole_C Fc)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGC

SEQ ID NO: 136 - protein, 226 aa, synthetic construct
  source 1..226: note = Fc (LALAPAknob Fc)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 228 aa, synthetic construct
  source 1..228: note = Fc (LALAPAknob_C Fc)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 138 - protein, 106 aa, synthetic construct [sequence omitted]

SEQ ID NO: 139 - protein, 100 aa, synthetic construct
  source 1..100: note = CH2
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 140 - DNA, 100 bp, synthetic construct
  source 1..100: mol_type = other DNA; note = CD28Apt7-dimer forward sequence
gaattctaatacgactcactatagggagagaggaagagggatggggattagaccataggctcccaacccccataacccagaggtcgatagtactggatcc

SEQ ID NO: 141 - DNA, 100 bp, synthetic construct
  source 1..100: mol_type = other DNA; note = CD28Apt7-dimer reverse sequence
ggggggatccagtactatcgacctctgggttatgggggttgggagcctatggtctaatccccatccctcttcctctctcccggggggatccagtactatc

SEQ ID NO: 142 - DNA, 22 bp, synthetic construct
  source 1..22: mol_type = other DNA; note = 1018 ISS
tgactgtgaacgttcgagatga

SEQ ID NO: 143 - DNA, 19 bp, synthetic construct
  source 1..19: mol_type = other DNA; note = AEG35156
  modified_base 1: mod_base = OTHER; note = uracil
  modified_base 17..19: mod_base = OTHER; note = uracil
tgcaccctggataccattt

SEQ ID NO: 144 - DNA, 20 bp, synthetic construct
  source 1..20: mol_type = other DNA; note = Abetimus subunit 1
gtgtgtgtgtgtgtgtgtgt

SEQ ID NO: 145 - DNA, 20 bp, synthetic construct
  source 1..20: mol_type = other DNA; note = Abetimus subunit 2
cacacacacacacacacaca

SEQ ID NO: 146 - DNA, 20 bp, synthetic construct
  source 1..20: mol_type = other DNA; note = Abetimus subunit 3
cacacacacacacacacaca

SEQ ID NO: 147 - DNA, 20 bp, synthetic construct
  source 1..20: mol_type = other DNA; note = Abetimus subunit 4
cacacacacacacacacaca

SEQ ID NO: 148 - DNA, 20 bp, synthetic construct
  source 1..20: mol_type = other DNA; note = Abetimus subunit 5
cacacacacacacacacaca

SEQ ID NO: 149 - DNA, 20 bp, synthetic construct
  source 1..20: mol_type = other DNA; note = Abetimus subunit 6
gtgtgtgtgtgtgtgtgtgt

SEQ ID NO: 150 - DNA, 20 bp, synthetic construct
  source 1..20: mol_type = other DNA; note = Abetimus subunit 7
gtgtgtgtgtgtgtgtgtgt

SEQ ID NO: 151 - DNA, 20 bp, synthetic construct
  source 1..20: mol_type = other DNA; note = Abetimus subunit 8
gtgtgtgtgtgtgtgtgtgt

SEQ ID NO: 152 - DNA, 20 bp, synthetic construct
  source 1..20: mol_type = other DNA; note = afovirsen
ttgcttccatcttcctcgtc

SEQ ID NO: 153 - DNA, 25 bp, synthetic construct
  source 1..25: mol_type = other DNA; note = aganirsen
tatccggagggctcgccatgctgct

SEQ ID NO: 154 - DNA, 24 bp, synthetic construct
  source 1..24: mol_type = other DNA; note = agatolimod
tcgtcgttttgtcgttttgtcgtt

SEQ ID NO: 155 - DNA, 20 bp, synthetic construct
  source 1..20: mol_type = other DNA; note = alicaforsen
gcccaagctggcatccgtca

SEQ ID NO: 156 - DNA, 26 bp, synthetic construct
  source 1..26: mol_type = other DNA; note = AGR0100
ggtggtggtggttgtggtggtggtgg

SEQ ID NO: 157 - DNA, 20 bp, synthetic construct
  source 1..20: mol_type = other DNA; note = amlivirsen
  modified_base 18: mod_base = OTHER; note = uracil
gcagaggtgaagcgaagtgc

SEQ ID NO: 158 - DNA, 31 bp, synthetic construct
  source 1..31: mol_type = other DNA; note = pegnivacogin
  modified_base order(2,7,9,16,19,22,26): mod_base = OTHER; note = uracil
gtggactataccgcgtaatgctgcctccact

SEQ ID NO: 159 - DNA, 15 bp, synthetic construct
  source 1..15: mol_type = other DNA; note = anivamersen
cgcggtatagtccac

SEQ ID NO: 160 - DNA, 20 bp, synthetic construct
  source 1..20: mol_type = other DNA; note = apatorsen
gggacgcggcgctcggtcat

SEQ ID NO: 161 - DNA, 26 bp, synthetic construct
  source 1..26: mol_type = other DNA; note = aprinocarsen
ggtggtggtggttgtggtggtggtgg

SEQ ID NO: 162 - RNA, 17 nt, synthetic construct
  source 1..17: mol_type = other RNA; note = ZINTEVIR
gtggtgggtgggtgggt

SEQ ID NO: 163 - DNA, 32 bp, synthetic construct
  source 1..32: mol_type = other DNA; note = ARC19499
ggaatatacttggctcgttaggtgcgtatata

SEQ ID NO: 164 - DNA, 20 bp, synthetic construct
  source 1..20: mol_type = other DNA; note = archexin
gctgcatgatctccttggcg

SEQ ID NO: 165 - DNA, 21 bp, synthetic construct
  source 1..21: mol_type = other DNA; note = Asvasiran antisense
  modified_base order(2,3,7,8,9,11): mod_base = OTHER; note = uracil
cttgactttgctaagagcctt

SEQ ID NO: 166 - DNA, 21 bp, synthetic construct
  source 1..21: mol_type = other DNA; note = Asvasiran sense
  modified_base order(4,6,7,15): mod_base = OTHER; note = uracil
ggctcttagcaaagtcaagtt

SEQ ID NO: 167 - DNA, 20 bp, synthetic construct
  source 1..20: mol_type = other DNA; note = atesidorsen
  modified_base order(1,18,19): mod_base = OTHER; note = uracil
tcagggcattctttccattc

SEQ ID NO: 168 - RNA, 23 nt, synthetic construct
  source 1..23: mol_type = other RNA; note = ATU-027 antisense
agacttgaggacttcctggacaa

SEQ ID NO: 169 - RNA, 23 nt, synthetic construct
  source 1..23: mol_type = other RNA; note = ATU-027 sense
ttgtccaggaagtcctcaagtct

SEQ ID NO: 170 - DNA, 39 bp, synthetic construct
  source 1..39: mol_type = other DNA; note = avacincaptad pegol
  modified_base order(9,11,19,23,25,29,30,31,35): mod_base = OTHER; note = uracil
cgccgcggtctcaggcgctgagtctgagtttacctgcgt

SEQ ID NO: 171 - DNA, 19 bp, synthetic construct
  source 1..19: mol_type = other DNA; note = AVI-7537
gccatggttttttctcagg

SEQ ID NO: 172 - DNA, 16 bp, synthetic construct
  source 1..16: mol_type = other DNA; note = baliforsen
tcccgaatgtccgaca

SEQ ID NO: 173 - DNA, 21 bp, synthetic construct
  source 1..21: mol_type = other DNA; note = Bamosiran antisense
  modified_base order(3,4,6,10,12,15): mod_base = OTHER; note = uracil
cattgtgcatgtgatccagtt

SEQ ID NO: 174 - DNA, 21 bp, synthetic construct
  source 1..21: mol_type = other DNA; note = Bamosiran sense
  modified_base order(2,6,11,18): mod_base = OTHER; note = uracil
ctggatcacatgcacaatgtt

SEQ ID NO: 175 - DNA, 18 bp, synthetic construct
  source 1..18: mol_type = other DNA; note = Bazlitoran
  modified_base order(8,18): mod_base = OTHER; note = uracil
ctatctgtcgttctctgt

SEQ ID NO: 176 - DNA, 16 bp, synthetic construct
  source 1..16: mol_type = other DNA; note = beclanorsen
  modified_base 2: mod_base = OTHER; note = uracil
ctcccaacgtgcgcca

SEQ ID NO: 177 - DNA, 20 bp, synthetic construct
  source 1..20: mol_type = other DNA; note = bepirovirsen
gcagaggtgaagcgaagtgc

SEQ ID NO: 178 - DNA, 21 bp, synthetic construct
  source 1..21: mol_type = other DNA; note = Bevasiranib antisense
  modified_base 4: mod_base = OTHER; note = uracil
acctcaccaaggccagcactt

SEQ ID NO: 179 - DNA, 21 bp, synthetic construct
  source 1..21: mol_type = other DNA; note = Bevasiranib sense
  modified_base order(2,5,10,11,14,19): mod_base = OTHER; note = uracil
gtgctggccttggtgaggttt

SEQ ID NO: 180 - DNA, 23 bp, synthetic construct
  source 1..23: mol_type = other DNA; note = Brivoligide
ctacgcccaccgcccacgcatac

SEQ ID NO: 181 - DNA, 23 bp, synthetic construct
  source 1..23: mol_type = other DNA; note = Brivoligide
gtatgcgtgggcggtgggcgtag

SEQ ID NO: 182 - DNA, 22 bp, synthetic construct
  source 1..22: mol_type = other DNA; note = casimersen
caatgccatcctggagttcctg

SEQ ID NO: 183 - DNA, 24 bp, synthetic construct
  source 1..24: mol_type = other DNA; note = cavrotolimod
tcgtcgttttgtcgttttgtcgtt

SEQ ID NO: 184 - DNA, 25 bp, synthetic construct
  source 1..25: mol_type = other DNA; note = Cemdisiran antisense
  modified_base order(1,3,4,6,12,14,16,17,20,21,22,23): mod_base = OTHER; note = uracil
tattataaaaatatcttgctttttt

SEQ ID NO: 185 - RNA, 22 nt, synthetic construct
  source 1..22: mol_type = other RNA; note = Cemdisiran sense
aagcaagatatttttataatan

SEQ ID NO: 186 - DNA, 20 bp, synthetic construct
  source 1..20: mol_type = other DNA; note = cenersen
ccctgctcccccctggctcc

SEQ ID NO: 187 - DNA, 19 bp, synthetic construct
  source 1..19: mol_type = other DNA; note = cobitolimod
ggaacagttcgtccatggc

SEQ ID NO: 188 - DNA, 30 bp, synthetic construct
  source 1..30: mol_type = other DNA; note = CODA-001
gtaattgcggcaagaagaattgtttctgtc

SEQ ID NO: 189 - DNA, 16 bp, synthetic construct
  source 1..16: mol_type = other DNA; note = cofirasersen
  modified_base order(14,16): mod_base = OTHER; note = uracil
cccgatagctggttgt

SEQ ID NO: 190 - RNA, 19 nt, synthetic construct
  source 1..19: mol_type = other RNA; note = Cosdosiran antisense
gccagaatgtggaactcct

SEQ ID NO: 191 - RNA, 19 nt, synthetic construct
  source 1..19: mol_type = other RNA; note = Cosdosiran sense
aggagttccacattctggc

SEQ ID NO: 192 - DNA, 21 bp, synthetic construct
  source 1..21: mol_type = other DNA; note = CPG-8954
gggggggtgtcgcagcagggg

SEQ ID NO: 193 - DNA, 20 bp, synthetic construct
  source 1..20: mol_type = other DNA; note = cupabimod
ggagggaaatcccttcaagg

SEQ ID NO: 194 - DNA, 20 bp, synthetic construct
  source 1..20: mol_type = other DNA; note = cupabimod
ccttgaagggatttccctcc

SEQ ID NO: 195 - DNA, 21 bp, synthetic construct
  source 1..21: mol_type = other DNA; note = Custirsen
  modified_base order(18,21): mod_base = OTHER; note = uracil
cagcagcagagtcttcatcat

SEQ ID NO: 196 - DNA, 16 bp, synthetic construct
  source 1..16: mol_type = other DNA; note = danvatirsen
  modified_base 2: mod_base = OTHER; note = uracil
ctatttggatgtcagc

SEQ ID NO: 197 - RNA, 21 nt, synthetic construct
  source 1..21: mol_type = other RNA; note = Daplusiran antisense
gtggacttctctcaattttct

SEQ ID NO: 198 - RNA, 21 nt, synthetic construct
  source 1..21: mol_type = other RNA; note = Daplusiran sense
agaaaattgagagaagtccac

SEQ ID NO: 199 - RNA, 25 nt, synthetic construct
  source 1..25: mol_type = other RNA; note = dematirsen
gttgcctccggttctgaaggtgttc

SEQ ID NO: 200 - DNA, 20 bp, synthetic construct
  source 1..20: mol_type = other DNA; note = donidalorsen
tgcaagtctcttggcaaaca

SEQ ID NO: 201 - RNA, 20 nt, synthetic construct
  source 1..20: mol_type = other RNA; note = drisapersen
tcaaggaagatggcatttct

SEQ ID NO: 202 - DNA, 14 bp, synthetic construct
  source 1..14: mol_type = other DNA; note = Edifoligide
ctagatttcccgcg

SEQ ID NO: 203 - DNA, 14 bp, synthetic construct
  source 1..14: mol_type = other DNA; note = Edifoligide
gatccgcgggaaat

SEQ ID NO: 204 - DNA, 40 bp, synthetic construct
  source 1..40: mol_type = other DNA; note = egaptivon pegol
  modified_base order(4,9,13,14,30,34): mod_base = OTHER; note = uracil
gcgtgcagtgccttcggccgtgcggtgcctccgtcacgct

SEQ ID NO: 205 - DNA, 20 bp, synthetic construct
  source 1..20: mol_type = other DNA; note = EIF-4E
tgttatattcctggatcctt

SEQ ID NO: 206 - RNA, 33 nt, synthetic construct
  source 1..33: mol_type = other RNA; note = Eluforsen
atcataggaaacaccaaagatgatattttcttt

SEQ ID NO: 207 - RNA, 40 nt, synthetic construct
  source 1..40: mol_type = other RNA; note = emapticap pegol
gcacgtccctcaccggtgcaagtgaagccgtggctctgcg

SEQ ID NO: 208 - DNA, 20 bp, synthetic construct
  source 1..20: mol_type = other DNA; note = eplontersen
  modified_base order(1,3,4,17): mod_base = OTHER; note = uracil
tcttggttacatgaaatccc

SEQ ID NO: 209 - DNA, 30 bp, synthetic construct
  source 1..30: mol_type = other DNA; note = eteplirsen
ctccaacatcaaggaagatggcatttctag

SEQ ID NO: 210 - DNA, 20 bp, synthetic construct
  source 1..20: mol_type = other DNA; note = fesomersen
  modified_base order(18,19,20): mod_base = OTHER; note = uracil
acggcattggtgcacagttt

SEQ ID NO: 211 - RNA, 23 nt, synthetic construct
  source 1..23: mol_type = other RNA; note = Fitusiran antisense
ttgaagtaaatggtgttaaccag

SEQ ID NO: 212 - RNA, 21 nt, synthetic construct
  source 1..21: mol_type = other RNA; note = Fitusiran sense
ggttaacaccatttacttcaa

SEQ ID NO: 213 - DNA, 21 bp, synthetic construct
  source 1..21: mol_type = other DNA; note = fomivirsen
gcgtttgctcttcttcttgcg

SEQ ID NO: 214 - DNA, 18 bp, synthetic construct
  source 1..18: mol_type = other DNA; note = gataparsen
tgtgctattctgtgaatt

SEQ ID NO: 215 - RNA, 23 nt, synthetic construct
  source 1..23: mol_type = other RNA; note = Givosiran antisense
aatgatgagacactctttctggt

SEQ ID NO: 216 - RNA, 21 nt, synthetic construct
  source 1..21: mol_type = other RNA; note = Givosiran sense
cagaaagagtgtctcatctta

SEQ ID NO: 217 - DNA, 21 bp, synthetic construct
  source 1..21: mol_type = other DNA; note = GNKG-168
tcgtcgacgtcgttcgttctc

SEQ ID NO: 218 - DNA, 25 bp, synthetic construct
  source 1..25: mol_type = other DNA; note = golodirsen
gttgcctccggttctgaaggtgttc

SEQ ID NO: 219 - DNA, 20 bp, synthetic construct
  source 1..20: mol_type = other DNA; note = GPI-2A
ggttcttttggtccttgtct

SEQ ID NO: 220 - DNA, 20 bp, synthetic construct
  source 1..20: mol_type = other DNA; note = GTI-2040
ggctaaatcgctccaccaag

SEQ ID NO: 221 - DNA, 20 bp, synthetic construct
  source 1..20: mol_type = other DNA; note = GTI-2501
ctctagcgtcttaaagccga

SEQ ID NO: 222 - DNA, 24 bp, synthetic construct
  source 1..24: mol_type = other DNA; note = IMT-504
tcatcattttgtcattttgtcatt

SEQ ID NO: 223 - DNA, 22 bp, synthetic construct
  source 1..22: mol_type = other DNA; note = Inclisiran antisense
  modified_base order(2,8,10,12,13,16,17,18,19,21): mod_base = OTHER; note = uracil
ctagacctgttttgcttttgtn

SEQ ID NO: 224 - RNA, 23 nt, synthetic construct
  source 1..23: mol_type = other RNA; note = Inclisiran sense
acaaaagcaaaacaggtctagaa

SEQ ID NO: 225 - DNA, 20 bp, synthetic construct
  source 1..20: mol_type = other DNA; note = inotersen
  modified_base order(1,3,4,17): mod_base = OTHER; note = uracil
tcttggttacatgaaatccc

SEQ ID NO: 226 - DNA, 13 bp, synthetic construct
  source 1..13: mol_type = other DNA; note = imetelstat
tagggttagacaa

SEQ ID NO: 227 - DNA, 19 bp, synthetic construct
  source 1..19: mol_type = other DNA; note = lademirsen
  modified_base 13: mod_base = OTHER; note = uracil
acatcagtctgataagcta

SEQ ID NO: 228 - RNA, 44 nt, synthetic construct
  source 1..44: mol_type = other RNA; note = lexaptepid pegol
gcgccgtatgggattaagtaaatgaggagttggaggaagggcgc

SEQ ID NO: 229 - DNA, 26 bp, synthetic construct
  source 1..26: mol_type = other DNA; note = litenimod
taaacgttataacgttatgacgtcat

SEQ ID NO: 230 - RNA, 21 nt, synthetic construct
  source 1..21: mol_type = other RNA; note = Lumasiran antisense
gactttcatcctggaaatata

SEQ ID NO: 231 - RNA, 23 nt, synthetic construct
  source 1..23: mol_type = other RNA; note = Lumasiran sense
tatatttccaggatgaaagtcca

SEQ ID NO: 232 - DNA, 20 bp, synthetic construct
  source 1..20: mol_type = other DNA; note = mipomersen
  modified_base 4: mod_base = OTHER; note = uracil
gcctcagtctgcttcgcacc

SEQ ID NO: 233 - DNA, 15 bp, synthetic construct
  source 1..15: mol_type = other DNA; note = miravirsen
ccattgtcacactcc

SEQ ID NO: 234 - DNA, 21 bp, synthetic construct
  source 1..21: mol_type = other DNA; note = mongersen
gtcgccccttctccccgcagc

SEQ ID NO: 235 - RNA, 22 nt, synthetic construct
  source 1..22: mol_type = other RNA; note = Nedosiran antisense
tcagataaaaaggacaacatgg

SEQ ID NO: 236 - RNA, 36 nt, synthetic construct
  source 1..36: mol_type = other RNA; note = Nedosiran sense
atgttgtcctttttatctgagcagccgaaaggctgc

SEQ ID NO: 237 - DNA, 18 bp, synthetic construct
  source 1..18: mol_type = other DNA; note = nusinersen
tcacctttataatgctgg

SEQ ID NO: 238 - DNA, 18 bp, synthetic construct
  source 1..18: mol_type = other DNA; note = oblimersen
tctcccagcgtgcgccat

SEQ ID NO: 239 - RNA, 45 nt, synthetic construct
  source 1..45: mol_type = other RNA; note = olaptesed pegol
gcgtggtgtgatctagatgtattggctgatcctagtcaggtacgc

SEQ ID NO: 240 - RNA, 21 nt, synthetic construct
  source 1..21: mol_type = other RNA; note = Olpasiran antisense
cagccccttattgttatacga

SEQ ID NO: 241 - RNA, 21 nt, synthetic construct
  source 1..21: mol_type = other RNA; note = Olpasiran sense
tcgtataacaataaggggctg

SEQ ID NO: 242 - DNA, 21 bp, synthetic construct
  source 1..21: mol_type = other DNA; note = Patisiran antisense
  modified_base order(2,12,14,15,19): mod_base = OTHER; note = uracil
gtaaccaagagtattccattt

SEQ ID NO: 243 - DNA, 21 bp, synthetic construct
  source 1..21: mol_type = other DNA; note = Patisiran sense
  modified_base order(2,7,10,12,13,16,17): mod_base = OTHER; note = uracil
atggaatactcttggttactt

SEQ ID NO: 244 - DNA, 28 bp, synthetic construct
  source 1..28: mol_type = other DNA; note = Pegaptanib
  modified_base order(6,10,14,17,18,20,24): mod_base = OTHER; note = uracil
cggaatcagtgaatgcttatacatccgt

SEQ ID NO: 245 - DNA, 30 bp, synthetic construct
  source 1..30: mol_type = other DNA; note = Pegpleranib
  modified_base order(6,19,28): mod_base = OTHER; note = uracil
caggctacgcgtagagcatcatgatcctgt

SEQ ID NO: 246 - DNA, 20 bp, synthetic construct
  source 1..20: mol_type = other DNA; note = Pelacarsen
tgctccgttggtgcttgttc

SEQ ID NO: 247 - DNA, 18 bp, synthetic construct
  source 1..18: mol_type = other DNA; note = Prexigebersen
atatttggcgatggcttc

SEQ ID NO: 248 - DNA, 30 bp, synthetic construct; identical to SEQ ID NO: 209
  source 1..30: mol_type = other DNA; note = Radavirsen

SEQ ID NO: 249 - RNA, 25 nt, synthetic construct
  source 1..25: mol_type = other RNA; note = Remlarsen antisense
tagcaccatttgaaatcagtgtttt

SEQ ID NO: 250 - RNA, 23 nt, synthetic construct
  source 1..23: mol_type = other RNA; note = Remlarsen sense
aacactgtttacaaatggtccta

SEQ ID NO: 251 - RNA, 18 nt, synthetic construct
  source 1..18: mol_type = other RNA; note = Renadirsen
cctaccgtaacccgtcgc

SEQ ID NO: 252 - DNA, 20 bp, synthetic construct
  source 1..20: mol_type = other DNA; note = AVI-4126
acgttgaggggcatcgtcgc

SEQ ID NO: 253 - RNA, 21 nt, synthetic construct
  source 1..21: mol_type = other RNA; note = Revusiran antisense
tgggatttcatgtaaccaaga

SEQ ID NO: 254 - RNA, 23 nt, synthetic construct
  source 1..23: mol_type = other RNA; note = Revusiran sense
tcttggttacatgaaatcccatc

SEQ ID NO: 255 - skipped

SEQ ID NO: 256 - RNA, 20 nt, synthetic construct
  source 1..20: mol_type = other RNA; note = Rimigorsen
tcagcttctgttagccactg

SEQ ID NO: 257 - DNA, 24 bp, synthetic construct
  source 1..24: mol_type = other DNA; note = Rosomidnar
cacgcacgcgcatccccgcccgtg

SEQ ID NO: 258 - RNA, 20 nt, synthetic construct
  source 1..20: mol_type = other RNA; note = Suvodirsen
tcaaggaagatggcatttct

SEQ ID NO: 259 - DNA, 19 bp, synthetic construct
  source 1..19: mol_type = other DNA; note = Temavirsen
  modified_base order(7,10): mod_base = OTHER; note = uracil
acaccattgtcacactcca

SEQ ID NO: 260 - RNA, 19 nt, synthetic construct
  source 1..19: mol_type = other RNA; note = Teprasiran antisense
tgaagggtgaaatattctc

SEQ ID NO: 261 - RNA, 19 nt, synthetic construct
  source 1..19: mol_type = other RNA; note = Teprasiran sense
gagaatatttcacccttca

SEQ ID NO: 262 - RNA, 19 nt, synthetic construct
  source 1..19: mol_type = other RNA; note = Tivanisiran antisense
aagcgcatcttctacttca

SEQ ID NO: 263 - RNA, 19 nt, synthetic construct
  source 1..19: mol_type = other RNA; note = Tivanisiran sense
tgaagtagaagatgcgctt

SEQ ID NO: 264 - DNA, 20 bp, synthetic construct
  source 1..20: mol_type = other DNA; note = tofersen
  modified_base 20: mod_base = OTHER; note = uracil
caggatacatttctacagct

SEQ ID NO: 265 - DNA, 20 bp, synthetic construct
  source 1..20: mol_type = other DNA; note = tominersen
  modified_base 2: mod_base = OTHER; note = uracil
ctcagtaacattgacaccac

SEQ ID NO: 266 - DNA, 18 bp, synthetic construct
  source 1..18: mol_type = other DNA; note = trabedersen
cggcatgtctattttgta

SEQ ID NO: 267 - DNA, 25 bp, synthetic construct
  source 1..25: mol_type = other DNA; note = trecovirsen
tcttcctctctctacccacgctctc

SEQ ID NO: 268 - DNA, 30 bp, synthetic construct
  source 1..30: mol_type = other DNA; note = vidutolimod
gggggggggggacgatcgtcgggggggggg

SEQ ID NO: 269 - DNA, 21 bp, synthetic construct
  source 1..21: mol_type = other DNA; note = viltolarsen
cctccggttctgaaggtgttc

SEQ ID NO: 270 - DNA, 20 bp, synthetic construct
  source 1..20: mol_type = other DNA; note = volanesorsen
  modified_base order(4,5,16,17,18,20): mod_base = OTHER; note = uracil
agcttcttgtccagctttat

SEQ ID NO: 271 - DNA, 20 bp, synthetic construct
  source 1..20: mol_type = other DNA; note = vupanorsen
ggacattgccagtaatcgca

SEQ ID NO: 272 - RNA, 21 nt, synthetic construct
  source 1..21: mol_type = other RNA; note = Vutrisiran antisense
tgggatttcatgtaaccaaga

SEQ ID NO: 273 - RNA, 23 nt, synthetic construct
  source 1..23: mol_type = other RNA; note = Vutrisiran sense
tcttggttacatgaaatcccatc

SEQ ID NO: 274 - RNA, 20 nt, synthetic construct
  source 1..20: mol_type = other RNA; note = Rovanersen
ggcacaagggcacagacttc

SEQ ID NO: 275 - DNA, 20 bp, synthetic construct
  source 1..20: mol_type = other DNA; note = Lexanersen
  modified_base 2: mod_base = OTHER; note = uracil
gtgcacacagtagatgaggg

SEQ ID NO: 276 - DNA, 16 bp, synthetic construct
  source 1..16: mol_type = other DNA; note = Cepadacursen
aatgctacaaaaccca

SEQ ID NO: 277 - DNA, 16 bp, synthetic construct
  source 1..16: mol_type = other DNA; note = ISIS-863633
aataatctcatgtcag

SEQ ID NO: 278 - DNA, 26 bp, synthetic construct
  source 1..26: mol_type = other DNA; note = AS1411
ggtggtggtggttgtggtggtggtgg

SEQ ID NO: 279 - RNA, 22 nt, synthetic construct
  source 1..22: mol_type = other RNA; note = Belcesiran antisense
tttaagaagacaaagggtttgg

SEQ ID NO: 280 - RNA, 36 nt, synthetic construct
  source 1..36: mol_type = other RNA; note = Belcesiran sense
aaaccctttgtcttcttaaagcagccgaaaggctgc

SEQ ID NO: 281 - DNA, 20 bp, synthetic construct
  source 1..20: mol_type = other DNA; note = Cimderlirsen
ccacctttgggtgaatagca

SEQ ID NO: 282 - DNA, 16 bp, synthetic construct
  source 1..16: mol_type = other DNA; note = Frenlosirsen
  modified_base 14: mod_base = OTHER; note = uracil
agttgtaaatgagtcg

SEQ ID NO: 283 - RNA, 20 nt, synthetic construct
  source 1..20: mol_type = other RNA; note = LSP-GR3
gctaggtttacgggacctct

SEQ ID NO: 284 - DNA, 20 bp, synthetic construct
  source 1..20: mol_type = other DNA; note = Monarsen
ctgcgatattttcttgtacc

SEQ ID NO: 285 - RNA, 20 nt, synthetic construct
  source 1..20: mol_type = other RNA; note = Olezarsen
  modified_base order(4,5,16,17,18,20): mod_base = OTHER; note = uracil
agcttcttgtccagctttat

SEQ ID NO: 286 - DNA, 25 bp, synthetic construct
  source 1..25: mol_type = other DNA; note = PUL-042
tcgtcgtcgttcgaacgacgttgat

SEQ ID NO: 287 - RNA, 20 nt, synthetic construct
  source 1..20: mol_type = other RNA; note = Sapablursen
  modified_base order(2,3,4,20): mod_base = OTHER; note = uracil
ctttattccaaagggcagct

SEQ ID NO: 288 - RNA, 17 nt, synthetic construct
  source 1..17: mol_type = other RNA; note = Sepofarsen
ggtggatcacgagttca

SEQ ID NO: 289 - DNA, 18 bp, synthetic construct
  source 1..18: mol_type = other DNA; note = Tadnersen
  modified_base 17: mod_base = OTHER; note = uracil
gcccctagcgcgcgactc

SEQ ID NO: 290 - DNA, 11 bp, synthetic construct
  source 1..11: mol_type = other DNA; note = Tilsotolimod, chain 1
tcgaacgttcg

SEQ ID NO: 291 - DNA, 11 bp, synthetic construct
  source 1..11: mol_type = other DNA; note = Tilsotolimod, chain 2
tcgaacgttcg

SEQ ID NO: 292 - RNA, 21 nt, synthetic construct
  source 1..21: mol_type = other RNA; note = Tomligisiran antisense
taccaatttatgcctacagcg

SEQ ID NO: 293 - RNA, 21 nt, synthetic construct
  source 1..21: mol_type = other RNA; note = Tomligisiran sense
cgctgtaggcataaattggta

SEQ ID NO: 294 - DNA, 21 bp, synthetic construct
  source 1..21: mol_type = other DNA; note = TOP-1731
tcatgagtggcagctgcaatt

SEQ ID NO: 295 - RNA, 25 nt, synthetic construct
  source 1..25: mol_type = other RNA; note = Varodarsen
tttgccgctgcccaatgccatcctg

SEQ ID NO: 296 - DNA, 21 bp, synthetic construct
  source 1..21: mol_type = other DNA; note = VEGLIN 3
tggcttgaagatgtactcgat

SEQ ID NO: 297 - RNA, 23 nt, synthetic construct
  source 1..23: mol_type = other RNA; note = Zilebesiran
tgtactctcattgtggatgacga

SEQ ID NO: 298 - RNA, 22 nt, synthetic construct
  source 1..22: mol_type = other RNA; note = Zilebesiran
gtcatccacaatgagagtacan

SEQ ID NO: 299 - protein, 548 aa, synthetic construct
  source 1..548: note = acmucabtagene autoleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLP

SEQ ID NO: 300 - protein, 465 aa, synthetic construct
  source 1..465: note = anbalcabtagene autoleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 301 - protein, 467 aa, synthetic construct
  source 1..467: note = axicabtagene ciloleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 302 - protein, 465 aa, synthetic construct
  source 1..465: note = azercabtagene zapreleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 303 - protein, 467 aa, synthetic construct
  source 1..467: note = brexucabtagene autoleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGGSGGLITRLWCKLDSPASTKWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 304 - protein, 475 aa, synthetic construct
  source 1..475: note = cemacabtagene ansegedleucel
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 305 - protein, 484 aa, synthetic construct
  source 1..484: note = evoncabtagene pazurgedleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 306 - protein, 470 aa, synthetic construct
  source 1..470: note = inaticabtagene autoleucel
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSEFTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 307 - protein, 657 aa, synthetic construct
  source 1..657: note = lisocabtagene maraleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQV

SEQ ID NO: 308 - protein, 406 aa, synthetic construct
  source 1..406: note = obecabtagene autoleucel
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRR

SEQ ID NO: 309 - protein, 306 aa, synthetic construct
  source 1..306: note = anitocabtagene autoleucel
GSWSEFWARLGAIRERLDALGGSEAELAAFEKEIAAFESELQAYKGKGNPEVEKLRYTAGTIKRFLQAYRHNGGGGDGGGGSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 310 - protein, 467 aa, synthetic construct
  source 1..467: note = ciltacabtagene autoleucel
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 311 - protein, 1016 aa, synthetic construct
  source 1..1016: note = dalucabtagene autoleucel
MSLPVTALLLPLALLLHAARPDIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKRSGGGGSGGGGSGGGGSQVQLVQSGAEVKKPGASVKLSCKASGYAFSSYWMNWVRQAPGQSLEWLGQIWPGDGDTNYNGKFKGRVTITADESARTAYMELSSLRSGDTAVYFCARRETTTVGRYYYAMDYWGKGTLVTVSSDPTTTPAPRPPTPAPTIASQPLSLRPEACRPAAGGAVHTRGLDFACDIIIAIAVVGALLLVALIFGTASYLIRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAEGRGSLLTCGDVEENPGPMETDTLILWVLLLLVPGSTGDIVMTQSPATLSVSPGERATLSCRSSQSLVHSNGNTYLHWYQQKPGQAPRLLIYKVSNRFSGVPARFSGSGSGAEFTLTISSLQSEDFAVYYCSQSTHVPWTFGQGTRLEIKRSGGGGSGGGGSGGGGSEVQLVESGAEVKKPGSSVKVSCKASGYTFTNYWINWVRQAPGQGLEWMGNIYPSDSFTNYNQKFKDRVTITADKSTSTVYLELRNLRSDDTAVYYCTRDTQERSWYFDVWGQGTLVTVSSDPAGSDLGPQMLRELQETNAALQDVRELLRQQVREITFLKNTVMECDACGSGKKDPKIIAIAVVGALLLVALIFGTASYLIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 312 - protein, 472 aa, synthetic construct
  source 1..472: note = durcabtagene autoleucel
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 313 - protein, 480 aa, synthetic construct
  source 1..480: note = equecabtagene autoleucel
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 314 - protein, 501 aa, synthetic construct
  source 1..501: note = fencabtagene autoleucel
SRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 315 - protein, 462 aa, synthetic construct
  source 1..462: note = firicabtagene autoleucel
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 316 - protein, 316 aa, synthetic construct
  source 1..316: note = gavocabtagene autoleucel
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 317 - protein, 472 aa, synthetic construct
  source 1..472: note = idecabtagene vicleucel
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 318 - protein, 666 aa, synthetic construct
  source 1..666: note = itezocabtagene autoleucel
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 319 - protein, 487 aa, synthetic construct
  source 1..487: note = motacabtagene lurevgedleucel
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 320 - protein, 406 aa, synthetic construct; identical to SEQ ID NO: 308
  source 1..406: note = obecabtagene autoleucel

SEQ ID NO: 321 - protein, 1035 aa, synthetic construct
  source 1..1035: note = orvacabtagene autoleucel
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 322 - protein, 447 aa, synthetic construct
  source 1..447: note = plixacabtagene autoleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 323 - protein, 527 aa, synthetic construct
  source 1..527: note = pomlucabtagene autoleucel
DIQLTQSPSSLSASPGDRVTITCSASSSVSYMHWFQQKPGKAPKLWIYSTSNLASGVPARFSGSGSGTSYSLTISRLQPEDIATYYCQQRSSYPYTFGGGTKVEIKGGGGSGGGGSGGGGSEVQLVQSGAEVKKPGASVKVSCKASGYTFTDYNMDWVKQSPGQGLEWMGYIYPNNGGTGYNQKFKSKVTITVDTSTSTAYMELHSLRSEDTAVYYCATYGHYYGYMFAYWGQGTLVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 324 - protein, 691 aa, synthetic construct
  source 1..691: note = prizloncabtagene autoleucel
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGSTSGGGSGGGSGGGGSSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 325 - protein, 465 aa, synthetic construct
  source 1..465: note = rapcabtagene autoleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 326 - protein, 820 aa, synthetic construct
  source 1..820: note = relmacabtagene autoleucel
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 327 - protein, 348 aa, synthetic construct
  source 1..348: note = ribrecabtagene autoleucel
EVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 328 - protein, 473 aa, synthetic construct
  source 1..473: note = satricabtagene autoleucel
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 329 - protein, 467 aa, synthetic construct; identical to SEQ ID NO: 301
  source 1..467: note = tebrocabtagene autoleucel

SEQ ID NO: 330 - protein, 724 aa, synthetic construct
  source 1..724: note = tinocabtagene autoleucel
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARLPGYESAFDIWGQGTMVTVSSGGGGSQAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLGGSTSGSGKPGSGEGSTKGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQAGLFPYTFGGGTKVEIKFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 331 - protein, 476 aa, synthetic construct
  source 1..476: note = trovocabtagene autoleucel
DIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSGGGGSGGGGSEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 332 - protein, 467 aa, synthetic construct
  source 1..467: note = vadacabtagene leraleucel
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSVEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 333 - protein, 479 aa, synthetic construct
  source 1..479: note = varnimcabtagene autoleucel
NIVLTQSPASLAVSLGQRATISCRASESVDNFGNSFMHWYQQKSGQPPRLLIYIASNLESGVPARFSGSGSRTDFTLTIDPVEADDAATYYCHQNNEDPLTFGAGTKLELKGGGGSGGGGSGGGGSGGGGSHSQIQLQQSGAELVRPGSSVKISCKASGFAFSSYWMNWVKQRPGQGLEWIGQIYPGDGDTKYNVKFRGKATLTADESSSTAYIQLTSLTSEDSGVYFCARKRITAVITTVFDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 334 - protein, 487 aa, synthetic construct
  source 1..487: note = volamcabtagene durzigedleucel
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 335 - protein, 454 aa, synthetic construct
  source 1..454: note = zamtocabtagene autoleucel
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 336 - protein, 468 aa, synthetic construct
  source 1..468: note = zevorcabtagene autoleucel
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 337 - protein, 546 aa, synthetic construct
  source 1..546: note = afamitresgene autoleucel
KNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRA

SEQ ID NO: 338 - protein, 546 aa, synthetic construct
  source 1..546: note = besvatresgene autoleucel
KQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRA

SEQ ID NO: 339 - protein, 546 aa, synthetic construct; identical to SEQ ID NO: 338
  source 1..546: note = letetresgene autoleucel

SEQ ID NO: 340 - protein, 539 aa, synthetic construct
  source 1..539: note = mipetresgene autoleucel
NAGVTQTPKFQVLKTGQSMTLQCAQDMNHEYMSWYRQDPGMGLRLIHYSVAEGITDQGEVPNGYNVSRSTTEDFPLRLLSAAPSQTSVYFCASSYVGAAGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADCGFTSESYQQGVLSATILYEILLGKATLYAVLVSALVLMAMVKRKDSRGSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPQTGGSYIPTFGRGTSLIVHPYIQNPDPAVYQLRRSKSSDKSVCLFTDFDSQTNVSQSKDSDVYITDKTVLDMRSMDFKSNSAVAWSNKSDFACANAFNNSIIPEDTFFPSP

SEQ ID NO: 341 - protein, 540 aa, synthetic construct
  source 1..540: note = olitresgene autoleucel
QKEVEQNSGPLSVPEGAIASLNCTYSDRGSQSFFWYRQYSGKSPELIMSIYSNGDKEDGRFTAQLNKASQYVSLLIRDSQPSDSATYLCAVRGTGRRALTFGSGTRLQVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMSLGLLCCGVFSLLWAGPVNAGVTQTPKFRVLKTGQSMTLLCAQDMNHDYMYWYRQDPGMGLRLIHYSVGEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRA

SEQ ID NO: 342 - protein, 546 aa, synthetic construct; identical to SEQ ID NO: 338
  source 1..546: note = suvutresgene autoleucel

SEQ ID NO: 343 - protein, 254 aa, synthetic construct
  source 1..254: note = tacatresgene autoleucel
LKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVSRERIPEARAKRGSGATNFSLLKQAGDVEENPGPMATGSRTSLLLAFGLLCLPWLQEGSAIQNPDPAVYQLRDSKSSDKSVCLF

SEQ ID NO: 344 - protein, 465 aa, synthetic construct; identical to SEQ ID NO: 325
  source 1..465: note = tisagenlecleuce

SEQ ID NO: 345 - protein, 504 aa, synthetic construct
  source 1..504: note = Blinatumomab
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 346 - protein, 448 aa, synthetic construct
  source 1..448: note = Budoprutug Heavy Chain
VQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 347 - protein, 211 aa, synthetic construct
  source 1..211: note = Budoprutug Light Chain
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 450 aa, synthetic construct
  source 1..450: note = coltuximab ravtansine heavy chain
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 211 aa, synthetic construct
  source 1..211: note = coltuximab ravtansine light chain
EIVLTQSPAIMSASPGERVTMTCSASSGVNYMHWYQQKPGTSPRRWIYDTSKLASGVPARFSGSGSGTDYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 350 - protein, 450 aa, synthetic construct
  source 1..450: note = denintuzumab mafodotin heavy chain
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 213 aa, synthetic construct
  source 1..213: note = denintuzumab mafodotin light chain
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 352 - protein, 502 aa, synthetic construct
  source 1..502: note = Duvortuxizumab (chain 1)
ENVLTQSPATLSVTPGEKATITCRASQSVSYMHWYQQKPGQAPRLLIYDASNRASGVPSRFSGSGSGTDHTLTISSLEAEDAATYYCFQGSVYPFTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 271 aa, synthetic construct
  source 1..271: note = Duvortuxizumab (chain 2)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSSASTKGKVAACKEKVAALKEKVAALKEKVAALKE

SEQ ID NO: 354 - protein, 227 aa, synthetic construct
  source 1..227: note = Duvortuxizumab (chain 3)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 355 - protein, 1230 aa, synthetic construct
  source 1..1230: note = Emfizatamab heavy chain
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKGGGGSGGGGSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSGGGGSGGGGSEVQLVQSGAEVKKPGESLKISCKGSGYSFSSSWIGWVRQAPGKGLEWMGIIYPDDSDTRYSPSFQGQVTISADKSIRTAYLQWSSLKASDTAMYYCARHVTMIWGVIIDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 356 - protein, 214 aa, synthetic construct
  source 1..214: note = Emfizatamab light chain
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 219 aa, synthetic construct
  source 1..219: note = Englumafusp alfa (Chain 1)
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - protein, 451 aa, synthetic construct
  source 1..451: note = Englumafusp alfa (Chain 2)
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 359 - protein, 710 aa, synthetic construct
  source 1..710: note = Englumafusp alfa (Chain 3)
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 291 aa, synthetic construct
  source 1..291: note = Englumafusp alfa (Chain 4)
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 361 - protein, 451 aa, synthetic construct
  source 1..451: note = Inebilizumab heavy chain
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 218 aa, synthetic construct
  source 1..218: note = Inebilizumab light chain
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 363 - protein, 449 aa, synthetic construct
  source 1..449: note = Loncastuximab heavy chain
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 364 - protein, 211 aa, synthetic construct
  source 1..211: note = Loncastuximab light chain
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 365 - protein, 451 aa, synthetic construct
  source 1..451: note = Obexelimab heavy chain
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 219 aa, synthetic construct
  source 1..219: note = Obexelimab light chain
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 367 - protein, 451 aa, synthetic construct
  source 1..451: note = Tafasitamab heavy chain
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 368 - protein, 219 aa, synthetic construct; identical to SEQ ID NO: 366
  source 1..219: note = Tafasitamab light chain

SEQ ID NO: 369-370 - skipped (2 entries)

SEQ ID NO: 371 - protein, 445 aa, synthetic construct
  source 1..445: note = Zeripatamig (Chain 1)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 372 - protein, 215 aa, synthetic construct
  source 1..215: note = Zeripatamig (Chain 2)
DIQMTQSPSSLSASVGDRVTITCRASQSISSYLNWYQQKPGKAPKLLIYAASSLQSGVPSRFSGSGSGTDFTLTISSLQPEDFATYYCQQMHPRAPKTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 373 - protein, 218 aa, synthetic construct
  source 1..218: note = Zeripatamig (Chain 3)
NFMLTQPHSVSESPGKTVTISCTRSSGSIEDKYVQWYQQRPGSSPTIVIYYDNERPSGVPDRFSGSIDSSSNSASLTISGLKTEDEADYYCQTYDQSLYGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 374 - protein, 451 aa, synthetic construct
  source 1..451: note = Divozilimab heavy chain
EVQLVQPGAEVVKPGASVKVSCKASGYTFTSYNMHWVRQAPGRGLEWMGAIYPGNGDTSYNQKFKGRVTMTRDKSTSTVYMELSSLRSEDTAVYYCARSTYYGGDWYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 213 aa, synthetic construct
  source 1..213: note = Divozilimab light chain
QIVLSQSPAILSASPGERVTLTCRASSSVSYIHWFQQKPGKAPKPLIYATSNLASGVPSRFSGSGSGTDFSLTISRVEPEDFAVYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 376 - protein, 454 aa, synthetic construct
  source 1..454: note = Epcoritamab (Chain 1)
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 377 - protein, 215 aa, synthetic construct
  source 1..215: note = Epcoritamab (Chain 2)
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 378 - protein, 451 aa, synthetic construct
  source 1..451: note = Epcoritamab (Chain 3)
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 379 - protein, 214 aa, synthetic construct
  source 1..214: note = Epcoritamab (Chain 4)
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 380-381 - skipped (2 entries)

SEQ ID NO: 382 - protein, 622 aa, synthetic construct
  source 1..622: note = Eramkafusp alfa heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 383 - protein, 213 aa, synthetic construct
  source 1..213: note = Eramkafusp alfa light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 384 - protein, 449 aa, synthetic construct
  source 1..449: note = Glofitamab, chain 1
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 385 - protein, 232 aa, synthetic construct
  source 1..232: note = Glofitamab, chain 2
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 386 - protein, 674 aa, synthetic construct
  source 1..674: note = Glofitamab, chain 3
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 387 - protein, 219 aa, synthetic construct
  source 1..219: note = Glofitamab, chain 4
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 388 - protein, 443 aa, synthetic construct
  source 1..443: note = Ibritumomab tiuxetan, HC
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 389 - protein, 209 aa, synthetic construct
  source 1..209: note = Ibritumomab tiuxetan, LC
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRADAAPTVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 390 - protein, 575 aa, synthetic construct
  source 1..575: note = Imvotamab, chain 1
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 391 - protein, 219 aa, synthetic construct
  source 1..219: note = Imvotamab, chain 2
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 392 - protein, 993 aa, synthetic construct
  source 1..993: note = Imvotamab, chain 3
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 393 - protein, 449 aa, synthetic construct
  source 1..449: note = Mosunetuzumab
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 394 - protein, 219 aa, synthetic construct
  source 1..219: note = Obinutuzumab, Chain 1
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 395 - protein, 452 aa, synthetic construct
  source 1..452: note = Obinutuzumab, Chain 2
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 396 - protein, 213 aa, synthetic construct
  source 1..213: note = Obinutuzumab, Chain 3
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 397 - protein, 449 aa, synthetic construct
  source 1..449: note = Obinutuzumab, Chain 4
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 398 - protein, 218 aa, synthetic construct
  source 1..218: note = Ocaratuzumab
DIVMTQTPLSLPVTPGEPASISCRSSKSLLHSNGITYLYWYLQKPGQSPQLLIYQMSNLVSGVPDRFSGSGSGTDFTKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 399 - protein, 450 aa, synthetic construct
  source 1..450: note = Ocaratuzumab, Chain 1
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKIKDTLMISRTPEVTCVVVDVSHEDPEVKFNWYVDGVEVHNAKTKPREEQYNSTYRVVSVLTVLHQDWLNGKEYKCKVSNKALPAPIEKTISKQKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 400 - protein, 213 aa, synthetic construct
  source 1..213: note = Ocrelizumab, Chain 2
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 401 - protein, 452 aa, synthetic construct
  source 1..452: note = Ocrelizumab, Chain 3
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 402 - protein, 212 aa, synthetic construct
  source 1..212: note = Ocrelizumab, Chain 4
DIQMTQSPSSLSASVGDVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 403 - protein, 453 aa, synthetic construct
  source 1..453: note = Odronextamab/ofatumumab, Chain 1
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 404 - protein, 215 aa, synthetic construct
  source 1..215: note = Odronextamab/ofatumumab, Chain 2
AEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 405 - protein, 452 aa, synthetic construct
  source 1..452: note = Odronextamab/ofatumumab, Chain 3
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 406 - protein, 214 aa, synthetic construct; identical to SEQ ID NO: 379
  source 1..214: note = Odronextamab/ofatumumab, Chain 4

SEQ ID NO: 407 - protein, 450 aa, synthetic construct
  source 1..450: note = Plamotamab, HC
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 408 - protein, 213 aa, synthetic construct
  source 1..213: note = Plamotamab, LC
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 409 - protein, 451 aa, synthetic construct
  source 1..451: note = Ripertamab, HC
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 410 - protein, 213 aa, synthetic construct; identical to SEQ ID NO: 383
  source 1..213: note = Ripertamab, LC

SEQ ID NO: 411 - protein, 451 aa, synthetic construct; identical to SEQ ID NO: 409
  source 1..451: note = Rituximab, HC

SEQ ID NO: 412 - protein, 213 aa, synthetic construct; identical to SEQ ID NO: 383
  source 1..213: note = Rituximab, LC

SEQ ID NO: 413 - protein, 451 aa, synthetic construct
  source 1..451: note = Nofetumomab merpentan, HC
QIQLVQSGPVLKQPGETVKISCKASGYTFTNYGMNWVKQAPGKGLKWMGWINTYTGESTYADDFKGRFAFSLETFVTTACLQINNLKNEDTATYFCARWGPHTAPYSMDNWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 414 - protein, 220 aa, synthetic construct
  source 1..220: note = Nofetumomab merpentan, LC
QLVLTQSSSASFSLGASAKLTCTLSSQHSTYTIEWYQQQPLKPPKYVMELKKDGSHSTGDGIPDRFSGSSSGADRYLRISNIQPEDEAIYICGMGNKIKEQFVYVFGGGTKVTVLGQPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 415 - protein, 451 aa, synthetic construct; identical to SEQ ID NO: 413
  source 1..451: note = Tositumomab, HC

SEQ ID NO: 416 - protein, 220 aa, synthetic construct; identical to SEQ ID NO: 414
  source 1..220: note = Tositumomab, LC

SEQ ID NO: 417 - protein, 448 aa, synthetic construct
  source 1..448: note = Ublituximab, HC
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 418 - protein, 213 aa, synthetic construct
  source 1..213: note = Ublituximab, LC
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 419 - protein, 451 aa, synthetic construct
  source 1..451: note = Veltuzumab, HC
QVQLQQSGAEVKKPGSSVKVSCKASGYTFTSYNMHWVKQAPGQGLEWIGAIYPGMGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 420 - protein, 213 aa, synthetic construct
  source 1..213: note = Veltuzumab, LC
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 421 - protein, 452 aa, synthetic construct
  source 1..452: note = Zuberitamab, HC
EVQLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 422 - protein, 213 aa, synthetic construct
  source 1..213: note = Zuberitamab, LC
DIELSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 423 - protein, 694 aa, synthetic construct
  source 1..694: note = MD2452
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGSSGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGSGSGGGGSASGSGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGSSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSASGGSGESNIKVLPTWSTPVQPMALIVLGGVAGLLLFIGLGIFFCVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 424 - protein, 732 aa, synthetic construct
  source 1..732: note = MD2740
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGSSGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGSGSGGGGSASGSGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGSSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSASGGSGSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 425 - protein, 500 aa, synthetic construct
  source 1..500: note = Extracellular antigen recognition domain of MD2452 and MD2740
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGSSGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGSGSGGGGSASGSGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGSSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSASGGSG

SEQ ID NO: 426 - protein, 20 aa, synthetic construct
  source 1..20: note = Extracellular antigen recognition domain linker of MD2452 and MD2740
GGGGSGGSGSGGGGSASGSG

SEQ ID NO: 427 - protein, 18 aa, synthetic construct
  source 1..18: note = CD4 spacer - CD4 isoform 1, 381..398
ESNIKVLPTWSTPVQPMA

SEQ ID NO: 428 - protein, 10 aa, synthetic construct
  source 1..10: note = CD28 spacer - CD28 isoform 4, 157..166
SPLFPGPSKP

SEQ ID NO: 429 - protein, 9 aa, synthetic construct
  source 1..9: note = IgG2 spacer, hinge derived
KPCPPCKCP

SEQ ID NO: 430 - protein, 11 aa, synthetic construct
  source 1..11: note = IgG2 spacer, hinge derived
KPCPPCKCPAP

SEQ ID NO: 431 - protein, 15 aa, synthetic construct
  source 1..15: note = IgG2 spacer, hinge derived
GPTIKPCPPCKCPAP

SEQ ID NO: 432 - protein, 64 aa, synthetic construct
  source 1..64: note = IgG3 spacer, hinge derived
ELKTPLGDTTHTCPRCPEPKSCDTPPPCPRCPEPKSCDTPPPCPRCPEPKSCDTPPPCPRCPAP

SEQ ID NO: 433 - protein, 52 aa, synthetic construct
  source 1..52: note = IgG3 spacer, hinge derived
CPRCPEPKSCDTPPPCPRCPEPKSCDTPPPCPRCPEPKSCDTPPPCPRCPAP

SEQ ID NO: 434 - protein, 47 aa, synthetic construct
  source 1..47: note = IgG3 spacer, hinge derived
EPKSCDTPPPCPRCPEPKSCDTPPPCPRCPEPKSCDTPPPCPRCPAP

SEQ ID NO: 435 - protein, 32 aa, synthetic construct
  source 1..32: note = IgG3 spacer, hinge derived
EPKSCDTPPPCPRCPEPKSCDTPPPCPRCPAP

SEQ ID NO: 436 - protein, 22 aa, synthetic construct
  source 1..22: note = IgG3 spacer, hinge derived
CPRCPEPKSCDTPPPCPRCPAP

SEQ ID NO: 437 - protein, 17 aa, synthetic construct
  source 1..17: note = IgG3 spacer, hinge derived
EPKSCDTPPPCPRCPAP

SEQ ID NO: 438 - protein, 17 aa, synthetic construct
  source 1..17: note = IgG3 spacer, hinge derived
EPKSCDKTHTCPPCPAP

SEQ ID NO: 439 - protein, 16 aa, synthetic construct
  source 1..16: note = IgG1 spacer, hinge derived
PKSCDKTHTCPPCPAP

SEQ ID NO: 440 - protein, 8 aa, synthetic construct
  source 1..8: note = IgG3 spacer, hinge derived
PCPRCPAP

SEQ ID NO: 441 - protein, 11 aa, synthetic construct
  source 1..11: note = IgG2 spacer, hinge derived
PCPAPPVAGPS

SEQ ID NO: 442 - protein, 7 aa, synthetic construct
  source 1..7: note = IgG4 spacer, hinge derived
PPKPKDT

SEQ ID NO: 443 - protein, 28 aa, synthetic construct
  source 1..28: note = IgA1 spacer, hinge derived
TVPCPVPSTPPTPSPSTPPTPSPSCCHP

SEQ ID NO: 444 - protein, 10 aa, synthetic construct
  source 1..10: note = IgA1 spacer, hinge derived
TVPCPVPSTP

SEQ ID NO: 445 - protein, 10 aa, synthetic construct
  source 1..10: note = IgA1 spacer, hinge derived
TPSPSTPPTP

SEQ ID NO: 446 - protein, 9 aa, synthetic construct
  source 1..9: note = IgA1 spacer, hinge derived
TPSPSCCHP

SEQ ID NO: 447 - protein, 15 aa, synthetic construct
  source 1..15: note = IgA1 spacer, hinge derived
VPCPVPPPPPCCHPR

SEQ ID NO: 448 - protein, 7 aa, synthetic construct
  source 1..7: note = IgA1 spacer, hinge derived
VPCPVPP

SEQ ID NO: 449 - protein, 7 aa, synthetic construct
  source 1..7: note = IgA1 spacer, hinge derived
PPPCCHP

SEQ ID NO: 450 - protein, 13 aa, synthetic construct
  source 1..13: note = IgA1 spacer, hinge derived
PCPVPPPPPCCHP

SEQ ID NO: 451 - protein, 77 aa, synthetic construct
  source 1..77: note = IgD spacer, hinge derived
RWPESPKAQASSVPTAQPQAEGSLAKATTAPATTRNTGRGGEEKKKEKEKEEQEERETKTPECPSHTQPLGVYLLTP

SEQ ID NO: 452 - protein, 19 aa, synthetic construct
  source 1..19: note = IgD spacer, hinge derived
KTPECPSHTQPLGVYLLTP

SEQ ID NO: 453 - protein, 17 aa, synthetic construct
  source 1..17: note = IgD spacer, hinge derived
PECPSHTQPLGVYLLTP

SEQ ID NO: 454 - protein, 14 aa, synthetic construct
  source 1..14: note = IgD spacer, hinge derived
RWPESPKAQASSVP

SEQ ID NO: 455 - protein, 10 aa, synthetic construct
  source 1..10: note = IgE spacer, hinge derived
SVCSRDFTPP

SEQ ID NO: 456 - protein, 26 aa, synthetic construct
  source 1..26: note = IgM spacer, hinge derived
PLPVIAELPPKVSVFVPPRDGFFGNP

SEQ ID NO: 457 - protein, 10 aa, synthetic construct
  source 1..10: note = IgM spacer, hinge derived
PLPVIAELPP

SEQ ID NO: 458 - protein, 12 aa, synthetic construct
  source 1..12: note = IgM spacer, hinge derived
ELPPKVSVFVPP

SEQ ID NO: 459 - protein, 162 aa, synthetic construct
  source 1..162: note = 41BB spacer, 41BB derived
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSP

SEQ ID NO: 460 - protein, 81 aa, synthetic construct
  source 1..81: note = 41BB spacer, 41BB derived
DCKQGQELTKKGCKDCCFGTFNDQKRGICRPWTNCSLDGKSVLVNGTKERDVVCGPSPADLSPGASSVTPPAPAREPGHSP

SEQ ID NO: 461 - protein, 27 aa, synthetic construct
  source 1..27: note = 41BB spacer, 41BB derived
GPSPADLSPGASSVTPPAPAREPGHSP

SEQ ID NO: 462 - protein, 49 aa, synthetic construct
  source 1..49: note = CD8a spacer, CD8a derived
ASSGTTTPAPRPPTPAPTIASQPLSLRPEACRPAAGGAVHTRGLDFACD

SEQ ID NO: 463 - protein, 171 aa, synthetic construct
  source 1..171: note = CD27 spacer, CD27 derived
TPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 464 - protein, 70 aa, synthetic construct
  source 1..70: note = CD27 spacer, CD27 derived
PLPNPSLTARSSQALSPHPQPTHLPYVSEMLEARTAGHMQTLADFRQLPARTLSTHWPPQRSLCSSDFIR

SEQ ID NO: 465 - protein, 39 aa, synthetic construct
  source 1..39: note = CD28 spacer, CD28 derived
IEVMYPPPYLDNEKSNGTIIHVKGKHLCPSPLFPGPSKP

SEQ ID NO: 466 - protein, 12 aa, synthetic construct
  source 1..12: note = IgG4 spacer, IgG4 derived
ESKYGPPSPPSP

SEQ ID NO: 467 - protein, 30 aa, synthetic construct
  source 1..30: note = Dap10 spacer, Dap10 derived
QTTPGERSSLPAFYPGTSGSCSGCGSLSLP

SEQ ID NO: 468 - protein, 12 aa, synthetic construct
  source 1..12: note = IgG4 spacer, IgG4 derived
ESKYGPPSPPSP

SEQ ID NO: 469 - protein, 26 aa, synthetic construct
  source 1..26: note = ICOS spacer, ICOS derived
DPPPFKVTLTGGYLHIYESQLCCQLK

SEQ ID NO: 470 - protein, 48 aa, synthetic construct
  source 1..48: note = OX40 spacer, OX40 derived
EDRDPPATQPQETQGPPARPITVQPTEAWPRTSQGPSTRPVEVPGGRA

SEQ ID NO: 471 - protein, 119 aa, synthetic construct
  source 1..119: note = IgG4 spacer, IgG4 derived
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 472 - protein, 228 aa, synthetic construct
  source 1..228: note = IgG4 spacer, IgG4 CH2CH3
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 473 - protein, 23 aa, synthetic construct
  source 1..23: note = acmucabtagene autoleucel without binding domain
TSGGGGSLESGQVLLESNIKVLP

SEQ ID NO: 474 - protein, 223 aa, synthetic construct
  source 1..223: note = anbalcabtagene autoleucel without binding domain
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 475 - protein, 222 aa, synthetic construct
  source 1..222: note = axicabtagene ciloleucel without binding domain
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 476 - protein, 223 aa, synthetic construct
  source 1..223: note = azercabtagene zapreleucel without binding domain
TTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 477 - protein, 222 aa, synthetic construct; identical to SEQ ID NO: 475
  source 1..222: note = brexucabtagene autoleucel without binding domain

SEQ ID NO: 478 - protein, 227 aa, synthetic construct
  source 1..227: note = cemacabtagene ansegedleucel without binding domain
RSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 479 - protein, 239 aa, synthetic construct
  source 1..239: note = evoncabtagene pazurgedleucel without binding domain
AAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 480 - protein, 225 aa, synthetic construct
  source 1..225: note = inaticabtagene autoleucel without binding domain
EFTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 481 - protein, 412 aa, synthetic construct
  source 1..412: note = lisocabtagene maraleucel without binding domain
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQV

SEQ ID NO: 482 - protein, 166 aa, synthetic construct
  source 1..166: note = obecabtagene autoleucel without binding domain
RSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRR

SEQ ID NO: 483 - protein, 234 aa, synthetic construct
  source 1..234: note = anitocabtagene autoleucel without binding domain
GGGGDGGGGSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 484 - protein, 225 aa, synthetic construct
  source 1..225: note = ciltacabtagene autoleucel without binding domain
TSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 485 - protein, 235 aa, synthetic construct
  source 1..235: note = dalucabtagene autoleucel without binding domain
DPAGSDLGPQMLRELQETNAALQDVRELLRQQVREITFLKNTVMECDACGSGKKDPKIIAIAVVGALLLVALIFGTASYLIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 486 - protein, 223 aa, synthetic construct; identical to SEQ ID NO: 474
  source 1..223: note = durcabtagene autoleucel without binding domain

SEQ ID NO: 487 - protein, 237 aa, synthetic construct
  source 1..237: note = equecabtagene autoleucel without binding domain
FVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 488 - protein, 93 aa, synthetic construct
  source 1..93: note = fencabtagene autoleucel without binding domain
TSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 489 - protein, 226 aa, synthetic construct
  source 1..226: note = firicabtagene autoleucel without binding domain
AAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 490 - protein, 90 aa, synthetic construct
  source 1..90: note = gavocabtagene autoleucel without binding domain
VCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 491 - protein, 226 aa, synthetic construct
  source 1..226: note = idecabtagene vicleucel without binding domain
AAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 492 - protein, 421 aa, synthetic construct
  source 1..421: note = itezocabtagene autoleucel without binding domain
RSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 493 - protein, 242 aa, synthetic construct
  source 1..242: note = motacabtagene lurevgedleucel without binding domain
SAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 494 - protein, 167 aa, synthetic construct
  source 1..167: note = obecabtagene autoleucel without binding domain
KRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRR

SEQ ID NO: 495 - protein, 791 aa, synthetic construct
  source 1..791: note = orvacabtagene autoleucel without binding domain
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 496 - protein, 205 aa, synthetic construct
  source 1..205: note = plixacabtagene autoleucel without binding domain
AAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 497 - protein, 286 aa, synthetic construct
  source 1..286: note = pomlucabtagene autoleucel without binding domain
AAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 498 - protein, 194 aa, synthetic construct
  source 1..194: note = prizloncabtagene autoleucel without binding domain
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 499 - protein, 223 aa, synthetic construct
  source 1..223: note = rapcabtagene autoleucel without binding domain
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 500 - protein, 575 aa, synthetic construct
  source 1..575: note = relmacabtagene autoleucel without binding domain
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 501 - protein, 228 aa, synthetic construct
  source 1..228: note = ribrecabtagene autoleucel without binding domain
PAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 502 - protein, 227 aa, synthetic construct
  source 1..227: note = satricabtagene autoleucel without binding domain
RTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 503 - protein, 222 aa, synthetic construct; identical to SEQ ID NO: 475
  source 1..222: note = tebrocabtagene autoleucel without binding domain

SEQ ID NO: 504 - protein, 237 aa, synthetic construct; identical to SEQ ID NO: 487
  source 1..237: note = tinocabtagene autoleucel without binding domain

SEQ ID NO: 505 - protein, 233 aa, synthetic construct
  source 1..233: note = trovocabtagene autoleucel without binding domain
FVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 506 - protein, 223 aa, synthetic construct
  source 1..223: note = vadacabtagene leraleucel without binding domain
RAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSVEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 507 - protein, 224 aa, synthetic construct
  source 1..224: note = varnimcabtagene autoleucel without binding domain
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 508 - protein, 242 aa, synthetic construct; identical to SEQ ID NO: 493
  source 1..242: note = volamcabtagene durzigedleucel without binding domain

SEQ ID NO: 509 - protein, 225 aa, synthetic construct
  source 1..225: note = zevorcabtagene autoleucel without binding domain
RTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 510 - protein, 129 aa, synthetic construct
  source 1..129: note = afamitresgene autoleucel without binding domain
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRA

SEQ ID NO: 511 - protein, 223 aa, synthetic construct; identical to SEQ ID NO: 499
  source 1..223: note = Tisagenlecleucel without binding domain

SEQ ID NO: 512 - protein, 5 aa, synthetic construct
  source 1..5: note = Gly4-Ser linker
  REGION 1..5: note = sequence can be repeated 1 to n times
GGGGS

SEQ ID NO: 513 - protein, 20 aa, synthetic construct
  source 1..20: note = (Gly4Ser)4 linker
GGGGSGGGGSGGGGSGGGGS

SEQ ID NO: 514 - protein, 15 aa, synthetic construct
  source 1..15: note = (Gly4Ser)3 linker
GGGGSGGGGSGGGGS

SEQ ID NO: 515 - protein, 18 aa, synthetic construct
  source 1..18: note = CD4 CAR spacer
ESNIKVLPTWSTPVQPMA

SEQ ID NO: 516 - protein, 10 aa, synthetic construct
  source 1..10: note = CD28 CAR spacer
SPLFPGPSKP

SEQ ID NO: 517 - protein, 22 aa, synthetic construct
  source 1..22: note = CD4 TM
LIVLGGVAGLLLFIGLGIFFCV

SEQ ID NO: 518 - DNA, 81 bp, synthetic construct
  source 1..81: mol_type = other DNA; note = CD28 TM
ttttgggtgctggtggtggttggtggagtcctggcttgctatagcttgctagtaacagtggcctttattattttctgggtg

SEQ ID NO: 519 - protein, 27 aa, synthetic construct
  source 1..27: note = CD28 TM
FWVLVVVGGVLACYSLLVTVAFIIFWV

SEQ ID NO: 520 - DNA, 123 bp, synthetic construct
  source 1..123: mol_type = other DNA; note = CD28 IC
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 521 - protein, 41 aa, synthetic construct
  source 1..41: note = CD28 IC
RSKRSRLLHSDYMNMTPRRPGPTRKHYQPYAPPRDFAAYRS

SEQ ID NO: 522 - DNA, 126 bp, synthetic construct
  source 1..126: mol_type = other DNA; note = 4-1BB activation domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 523 - protein, 42 aa, synthetic construct
  source 1..42: note = 4-1BB activation domain
KRGRKKLLYIFKQPFMRPVQTTQEEDGCSCRFPEEEEGGCEL

SEQ ID NO: 524 - DNA, 336 bp, synthetic construct
  source 1..336: mol_type = other DNA; note = CD3zeta activation domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 525 - protein, 112 aa, synthetic construct
  source 1..112: note = CD3zeta activation domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 526 - protein, 176 aa, synthetic construct
  source 1..176: note = TM+IC region
LIVLGGVAGLLLFIGLGIFFCVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 527 - protein, 222 aa, synthetic construct
  source 1..222: note = TM+IC region
FWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 528 - protein, 154 aa, synthetic construct
  source 1..154: note = IC region
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 529 - protein, 195 aa, synthetic construct
  source 1..195: note = IC region
RSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 530 - protein, 5 aa, synthetic construct
  source 1..5: note = Gly-Ser linker/spacer
  REGION 1..4: note = Gly can be present 1-100 times in the sequence
  REGION 1..5: note = The entire sequence can occur 1-100 times
GGGGS

SEQ ID NO: 531 - protein, 5 aa, synthetic construct
  source 1..5: note = Gly-Ser linker/spacer
  REGION 1..4: note = Gly can be present 1-4 times in this sequence
  REGION 5: note = Ser can be present 0-1 times in this sequence
  REGION 1..5: note = This sequence can be repeated 1-50 times
GGGGS

SEQ ID NO: 532 - protein, 6 aa, synthetic construct
  source 1..6: note = Gly-Ser linker/spacer
  REGION 1..6: note = GlyAla can be present 1-100 times in this sequence
GAGAGA

SEQ ID NO: 533 - protein, 6 aa, synthetic construct
  source 1..6: note = Gly-Ser linker/spacer
  REGION 1..6: note = GlyGlySer can be present 1-100 times in this sequence
GGSGGS

SEQ ID NO: 534 - DNA, 1167 bp, synthetic construct
  source 1..1167: mol_type = other DNA; note = GFP construct, 5 UTR, coding sequence, 3 UTR, and polyA tail
taatacgactcactataggtcttctggtccccacagactcagagagaacccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaat

SEQ ID NO: 535 - DNA, 57 bp, synthetic construct
  source 1..57: mol_type = other DNA; note = GFP construct, 5' UTR
taatacgactcactataggtcttctggtccccacagactcagagagaacccgccacc

SEQ ID NO: 536 - DNA, 289 bp, synthetic construct
  source 1..289: mol_type = other DNA; note = GFP construct, 3 UTR
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 537 - DNA, 720 bp, synthetic construct
  source 1..720: mol_type = other DNA; note = GFP construct, ORF
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 538 - DNA, 101 bp, synthetic construct
  source 1..101: mol_type = other DNA; note = GFP construct, poly A tail
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaat

SEQ ID NO: 539 - protein, 12 aa, synthetic construct
  source 1..12: note = IgG4 spacer, IgG4 hinge
ESKYGPPCPPCP

SEQ ID NO: 540 - protein, 21 aa, synthetic construct
  source 1..21: note = CD8alpha signal peptide [CD8SP]
MALPVTALLLPLALLLHAARP

SEQ ID NO: 541 - protein, 720 aa, synthetic construct
  source 1..720: note = CD20CAR (MX2960), protein [CD8SP_ahCD20 [ofa]_FcLALPA_CD8bbz] (RN105)
MALPVTALLLPLALLLHAARPEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 542 - protein, 699 aa, synthetic construct
  source 1..699: note = CD20CAR (MX2960), protein [ahCD20 [ofa]_FcLALPA_CD8bbz] (RN105), mature (without CD8SP)
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 543 - DNA, 5019 bp, synthetic construct
  source 1..5019: mol_type = other DNA; note = CD20CAR (MX2960), RNA [ITSv1_5'hHBB_ahCD20 [ofa]_FcLALPA_CD8BBZ_3'xHBB] (RN105) MD4056
tctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgttgacattgattattgactagtgtaatacgactcactataaggacatttgcttctgacacaactgtgttcactagcaacctcaaacagacacctctagacaccatggctctgccggtcactgcgctgcttctcccactagccttgctgcttcacgctgcacgtccagagatcgtgctgacacaatcccccgctacactttccctgtccccaggcgagagagccactctcagttgcagggcttcacagtccgtgagttcttacctggcatggtatcagcagaagcctgggcaagccccacgcctgctgatctatgacgccagcaaccgcgctaccggaatcccagcgagattcagtggcagcgggagcggcacagacttcaccctcaccatcagctcactggagccagaggacttcgccgtgtattattgccagcagcgctcaaattggcccatcaccttcggccaggggacccggttagagatcaagggcggaggaggctccggcggtggggggagcgggggcgggggaagcgaggttcagcttgtggaaagcggcgggggactggtccagccaggccgttccctacggctctcttgtgcagcctctggatttaccttcaatgattacgcaatgcactgggtgagacaggcacccggaaagggcctggaatgggtgagtacaatttcctggaacagcggatcgataggatatgctgacagcgttaaaggtaggttcacaatttcacgggacaacgccaaaaagtcactgtacctgcaaatgaatagccttcgggctgaggacacggccctctactactgcgccaaggacatacagtacggtaactactactatggaatggatgtttggggccaagggacaaccgtgacagtaagtagcgcgagcggaggatcggacaagacacacacctgcccaccctgtccagcccctgaggctgcaggaggaccctctgtgttccttttcccaccgaaaccaaaggatacgcttatgatcagccgcacacctgaggtgacgtgcgtggtggtagacgtctcccacgaggacccggaagtcaaattcaactggtatgtagacggggtcgaggtgcacaatgcaaaaactaagccaagagaagaacagtacaactccacatacagggtcgtgagcgttttgactgtcctccatcaggactggctaaacgggaaagagtataagtgtaaagtgagtaataaggccctggccgccccaatcgagaagaccatctctaaggcaaaaggccaaccaagagagccgcaggtctacactctccctcctagccgggacgagttgactaaaaatcaggtttcgctgacatgccttgtgaagggattctaccctagcgacattgccgtggaatgggagagcaatggccaaccagaaaacaactacaaaaccaccccacctgtgcttgacagcgacggaagcttctttctctattctaagcttaccgtggataagagtcgatggcagcagggcaacgtgttttcctgttcagtcatgcacgaagcattgcacaatcattatacacaaaaatccctgtctctgtcgccaggcgccactaccacaccagcacccaggccacctaccccagccccaacaatcgccagtcagccactgagcctcaggcccgaggcttgccggcctgctgcggggggtgccgtccatacacggggattggacttcgcctgtgatatatatatctgggcacccctggctgggacctgcggcgtgctactgctgtctcttgttatcaccctctactgcaaacgaggccgcaaaaagttgttgtatattttcaaacagccgtttatgcgacccgtgcagaccacacaggaagaggacggctgctcctgccgatttccggaagaggaggagggagggtgtgaactccgcgtcaagttttccaggtctgcagatgcccccgcctacaagcaaggtcagaaccagctgtataacgagctgaatctgggtcgacgcgaagagtacgacgtcctggataaacggcggggacgcgacccagaaatgggcggcaagcctagaagaaagaacccacaagaggggctgtataacgagctacagaaagacaagatggccgaagcctattccgagatcgggatgaaaggggaacgcagaaggggaaaagggcacgacgggctctaccagggcctgagtaccgcgacaaaggatacctatgatgcactccatatgcaagccctcccccctagatgataggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgcggccgcgaattcggtacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctctcgacggatcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 544 - protein, 493 aa, synthetic construct
  source 1..493: note = CD79bCAR (MX2961), protein [CD8SP_anti-hCD79b [23F11wt]_CD8bbz] (RN111)
MALPVTALLLPLALLLHAARPDIQMTQSPSSLSASVGDRVTITCRASQSISSYLNWYQQKPRKAPKLLIYGASSLQSGVPSRFSGSGSGTDFTLTISSLQPEDFATYYCQQSYSTPLTFGGGTKVEIKGGGGSGGGGSGGGGSQVQLVESGGGVVQPGRSLRLSCAASGFTFSIYGMHWVRQAPGKGLEWMTTIWYDGSNKYYADSVKGRFTISRDNSKNTLYLQMNSLRAEDTAVYYCARAEELGEGVDYYYYYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 545 - protein, 472 aa, synthetic construct
  source 1..472: note = CD79bCAR (MX2961), protein [anti-hCD79b [23F11wt]_CD8bbz] (RN111), mature (without CD8SP)
DIQMTQSPSSLSASVGDRVTITCRASQSISSYLNWYQQKPRKAPKLLIYGASSLQSGVPSRFSGSGSGTDFTLTISSLQPEDFATYYCQQSYSTPLTFGGGTKVEIKGGGGSGGGGSGGGGSQVQLVESGGGVVQPGRSLRLSCAASGFTFSIYGMHWVRQAPGKGLEWMTTIWYDGSNKYYADSVKGRFTISRDNSKNTLYLQMNSLRAEDTAVYYCARAEELGEGVDYYYYYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 546 - DNA, 4337 bp, synthetic construct
  source 1..4337: mol_type = other DNA; note = CD79bCAR (MX2961), RNA [mRNA_5'hHBB_CD8SP_anti-hCD79b [23F11wt]_CD8BBZ_3'xHBB] (RN111) MD4143
tctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgttgacattgattattgactagtgtaatacgactcactataagaacatttgcttctgacacaactgtgttcactagcaacctcaaacagacacctctagacaccatggctttgccagtcacagcactgcttctcccactggctttgctgttgcacgccgcacgtccagacatccagatgacgcagagtccgtcctccctcagtgcctctgttggggaccgagtgaccattacatgccgtgcctcccaatcaatatcaagctatctaaactggtaccaacagaagccccggaaagcccccaaactgcttatctacggggcctctagccttcagtccggtgtgcccagcaggttctctggctctggtagcggaaccgattttacattgaccatctcatccctacagccggaagattttgctacctactactgccagcagagctactcgacaccactgacattcggcggagggaccaaggtggagataaagggtggcggcggcagcggtggtggcgggtcaggcgggggaggaagccaggtccagctcgtcgagtctgggggtggtgtggtacagcctgggaggagcctgcgactgagctgtgctgcaagcggctttactttcagcatttatggaatgcactgggtccggcaggcacccgggaagggtctcgagtggatgactacgatatggtatgacggcagcaataagtactacgccgattcagttaaggggagattcaccatctccagggacaactctaaaaatactctgtacttgcaaatgaatagcctgagagctgaagatactgccgtgtactattgtgctcgagctgaggagttaggagagggcgtggattattattattactatgggatggacgtttggggccaagggacaactgtgacagtctccagtaccacgactcccgctccacggcccccaacccctgcgccaactatcgcctctcagcctctgtccctgagacctgaggcctgccgccccgccgccggaggagccgttcatacccgcggccttgattttgcatgcgacatctacatttgggccccactagccggcacgtgcggagtgctgctcttaagtctggtaattaccctctactgtaaacggggacgcaaaaaactgctgtacattttcaagcagcctttcatgagaccggtgcagactacccaggaggaggacggatgtagttgtaggttccctgaagaggaggaaggcgggtgcgagctgcgcgtaaaattttcaagatcggctgacgcgcccgcatataagcagggccagaatcaactttacaacgaactcaacttaggccgccgggaagagtatgacgtgctggataaaaggcgaggccgggaccccgaaatgggcggaaagcctagaaggaaaaacccacaggaagggctgtataacgaacttcaaaaggataagatggccgaggcatattccgaaatcggcatgaagggagaacggaggcgcggcaagggtcatgacggactctaccaggggctgagtacagcaacaaaagacacctacgatgcgttgcacatgcaagcgctccctcccagatgataggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgcggccgcgaattcggtacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctctcgacggatcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 547 - protein, 494 aa, synthetic construct
  source 1..494: note = UTR_hHBB_xHBB_CD19CAR, protein [CD8SP_ahCD19 [hB43]_CD8bbz ] (RN068)
MALPVTALLLPLALLLHAARPDIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGGGSGGGGSGGGGSQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 548 - protein, 473 aa, synthetic construct
  source 1..473: note = UTR_hHBB_xHBB_CD19CAR, protein [ahCD19 [hB43]_CD8bbz ] (RN068), mature (without CD8SP)
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGGGSGGGGSGGGGSQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 549 - DNA, 4984 bp, synthetic construct
  source 1..4984: mol_type = other DNA; note = UTR_hHBB_xHBB_CD19CAR,, RNA [5'hHBB_CD19_CD8TM_3'xHBB] (RN068) MD3385
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagtgtaatacgactcactataagaacatttgcttctgacacaactgtgttcactagcaacctcaaacagacacctctagacaccatggccctgcctgtaactgcactgctgttgcctctggctctgctcctccacgcagcccgccccgatattcaactgacacagagcccctccttcctgagtgccagcgtgggggatagggtgacgatcacatgcaaggcttctcaatcagtggactacagcggagactcctacttgaactggtatcagcaaaagccgggtaaggcccccaagctcctgatctatgatgcgagcaatttggtctccggcgtcccctctaggtttagcggctcaggaagcggcaccgaatttaccctcaccatcagttccctgcagcccgaggattttgcaacgtactattgccagcagagcactgagaacccttggaccttcgggggcggaactaagttagagatcaaaggcggtggagggagcggcggaggcgggtctgggggaggcggtagccaagtgcagcttgtgcaaagtggtgcggaagtcaagaaaccaggctcctcagtcaaagtgtcgtgtaaagcctccgggtacgcctttagctcttactggatgaattgggtcaggcaggcccctggccaaggcctcgaatggatgggtcagatttggccaggcgatagcgacactaattatgctcaaaaattccaggggagagtaaccataacagctgacgaatccacttccacagcctatatggaattaagttcacttcgttctgaggatactgcagtgtattattgcgcccgaagggagaccaccacagtgggccggtactattacgccatggactactggggacagggaaccactgttacagtaagttctactaccacaccagctcctcggcctcctacaccggcccccacaatcgcatcacagcccctcagcctgcggcccgaggcgtgccggccagcagctggcggagccgttcacacccgagggctggacttcgcctgcgacatttatatatgggcaccactcgcagggacctgtggggtcttgctcctgtcccttgtgatcacgctttattgtaaaagaggtagaaaaaagttgttatacattttcaagcagcctttcatgaggcccgtgcagacaacgcaagaagaggatggttgctcttgtagattcccagaggaggaggagggaggctgtgagctccgggtgaagtttagtcgatcggctgatgctcccgcctacaagcagggacagaaccagctttacaatgaactgaacctaggtcgcagagaggagtacgatgttctggacaagagacgcggacgtgacccagaaatgggcggaaaaccgcgccggaaaaacccacaggaaggcctgtataacgaactacagaaagacaagatggctgaggcctacagcgagataggaatgaaaggcgaaaggcgccgagggaagggacacgatggcctgtaccaggggctgtcaaccgcgaccaaggacacttacgacgcactgcatatgcaggctctaccacctcgctgataggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgcggccgcgaattcggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 550 - protein, 494 aa, synthetic construct; identical to SEQ ID NO: 547
  source 1..494: note = UTR_hHBB_hHBB_CD19CAR, protein [CD8SP_ahCD19 [hB43]_CD8bbz] (RN082)

SEQ ID NO: 551 - protein, 473 aa, synthetic construct; identical to SEQ ID NO: 548
  source 1..473: note = UTR_hHBB_hHBB_CD19CAR, protein [ahCD19 [hB43]_CD8bbz] (RN082), mature (without CD8SP)

SEQ ID NO: 552 - DNA, 4974 bp, synthetic construct
  source 1..4974: mol_type = other DNA; note = UTR_hHBB_hHBB_CD19CAR, RNA [5'hHBB_CD19_CD8TM_3'hHBB] (RN082) MD3495
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagtgtaatacgactcactataagaacatttgcttctgacacaactgtgttcactagcaacctcaaacagacacctctagacaccatggccctgcctgtaactgcactgctgttgcctctggctctgctcctccacgcagcccgccccgatattcaactgacacagagcccctccttcctgagtgccagcgtgggggatagggtgacgatcacatgcaaggcttctcaatcagtggactacagcggagactcctacttgaactggtatcagcaaaagccgggtaaggcccccaagctcctgatctatgatgcgagcaatttggtctccggcgtcccctctaggtttagcggctcaggaagcggcaccgaatttaccctcaccatcagttccctgcagcccgaggattttgcaacgtactattgccagcagagcactgagaacccttggaccttcgggggcggaactaagttagagatcaaaggcggtggagggagcggcggaggcgggtctgggggaggcggtagccaagtgcagcttgtgcaaagtggtgcggaagtcaagaaaccaggctcctcagtcaaagtgtcgtgtaaagcctccgggtacgcctttagctcttactggatgaattgggtcaggcaggcccctggccaaggcctcgaatggatgggtcagatttggccaggcgatagcgacactaattatgctcaaaaattccaggggagagtaaccataacagctgacgaatccacttccacagcctatatggaattaagttcacttcgttctgaggatactgcagtgtattattgcgcccgaagggagaccaccacagtgggccggtactattacgccatggactactggggacagggaaccactgttacagtaagttctactaccacaccagctcctcggcctcctacaccggcccccacaatcgcatcacagcccctcagcctgcggcccgaggcgtgccggccagcagctggcggagccgttcacacccgagggctggacttcgcctgcgacatttatatatgggcaccactcgcagggacctgtggggtcttgctcctgtcccttgtgatcacgctttattgtaaaagaggtagaaaaaagttgttatacattttcaagcagcctttcatgaggcccgtgcagacaacgcaagaagaggatggttgctcttgtagattcccagaggaggaggagggaggctgtgagctccgggtgaagtttagtcgatcggctgatgctcccgcctacaagcagggacagaaccagctttacaatgaactgaacctaggtcgcagagaggagtacgatgttctggacaagagacgcggacgtgacccagaaatgggcggaaaaccgcgccggaaaaacccacaggaaggcctgtataacgaactacagaaagacaagatggctgaggcctacagcgagataggaatgaaaggcgaaaggcgccgagggaagggacacgatggcctgtaccaggggctgtcaaccgcgaccaaggacacttacgacgcactgcatatgcaggctctaccacctcgctgataggatccgctcgctttcttgctgtccaatttctattaaaggttcctttgttccctaagtccaactactaaactgggggatattatgaagggccttgagcatctggattctgcctaataaaaaacatttattttcattgcgatatccacctgcgccgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgcggccgcgaattcggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 553 - protein, 494 aa, synthetic construct; identical to SEQ ID NO: 547
  source 1..494: note = UTR_TEV_xHBB _CD19CAR, protein [CD8SP_ahCD19 [hB43]_CD8bbz] (RN083)

SEQ ID NO: 554 - protein, 473 aa, synthetic construct; identical to SEQ ID NO: 548
  source 1..473: note = UTR_TEV_xHBB _CD19CAR, protein [ahCD19 [hB43]_CD8bbz] (RN083), mature (without CD8SP)

SEQ ID NO: 555 - DNA, 5073 bp, synthetic construct
  source 1..5073: mol_type = other DNA; note = UTR_TEV_xHBB _CD19CAR, RNA [5'TEV_CD19_CD8TM_3'xHBB] (RN083) MD3496
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagtgtaatacgactcactataagcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagctctagacaccatggccctgcctgtaactgcactgctgttgcctctggctctgctcctccacgcagcccgccccgatattcaactgacacagagcccctccttcctgagtgccagcgtgggggatagggtgacgatcacatgcaaggcttctcaatcagtggactacagcggagactcctacttgaactggtatcagcaaaagccgggtaaggcccccaagctcctgatctatgatgcgagcaatttggtctccggcgtcccctctaggtttagcggctcaggaagcggcaccgaatttaccctcaccatcagttccctgcagcccgaggattttgcaacgtactattgccagcagagcactgagaacccttggaccttcgggggcggaactaagttagagatcaaaggcggtggagggagcggcggaggcgggtctgggggaggcggtagccaagtgcagcttgtgcaaagtggtgcggaagtcaagaaaccaggctcctcagtcaaagtgtcgtgtaaagcctccgggtacgcctttagctcttactggatgaattgggtcaggcaggcccctggccaaggcctcgaatggatgggtcagatttggccaggcgatagcgacactaattatgctcaaaaattccaggggagagtaaccataacagctgacgaatccacttccacagcctatatggaattaagttcacttcgttctgaggatactgcagtgtattattgcgcccgaagggagaccaccacagtgggccggtactattacgccatggactactggggacagggaaccactgttacagtaagttctactaccacaccagctcctcggcctcctacaccggcccccacaatcgcatcacagcccctcagcctgcggcccgaggcgtgccggccagcagctggcggagccgttcacacccgagggctggacttcgcctgcgacatttatatatgggcaccactcgcagggacctgtggggtcttgctcctgtcccttgtgatcacgctttattgtaaaagaggtagaaaaaagttgttatacattttcaagcagcctttcatgaggcccgtgcagacaacgcaagaagaggatggttgctcttgtagattcccagaggaggaggagggaggctgtgagctccgggtgaagtttagtcgatcggctgatgctcccgcctacaagcagggacagaaccagctttacaatgaactgaacctaggtcgcagagaggagtacgatgttctggacaagagacgcggacgtgacccagaaatgggcggaaaaccgcgccggaaaaacccacaggaaggcctgtataacgaactacagaaagacaagatggctgaggcctacagcgagataggaatgaaaggcgaaaggcgccgagggaagggacacgatggcctgtaccaggggctgtcaaccgcgaccaaggacacttacgacgcactgcatatgcaggctctaccacctcgctgataggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgcggccgcgaattcggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 556 - protein, 494 aa, synthetic construct; identical to SEQ ID NO: 547
  source 1..494: note = UTR_hHBA1_hAES/mtRNR1 _CD19CAR, protein [CD8SP_ahCD19 [hB43]_CD8bbz] NR1 (RN084)

SEQ ID NO: 557 - protein, 473 aa, synthetic construct; identical to SEQ ID NO: 548
  source 1..473: note = UTR_hHBA1_hAES/mtRNR1 _CD19CAR, protein [ahCD19 [hB43]_CD8bbz] NR1 (RN084), mature (without CD8SP)

SEQ ID NO: 558 - DNA, 5097 bp, synthetic construct
  source 1..5097: mol_type = other DNA; note = UTR_hHBA1_hAES/mtRNR1 _CD19CAR, RNA [5'HBA1_CD19_CD8TM_3'hAES+mtRNR1] NR1 (RN084) MD3497
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagtgtaatacgactcactataagatcttctggtccccacagactcagagagaacccgccaccatggccctgcctgtaactgcactgctgttgcctctggctctgctcctccacgcagcccgccccgatattcaactgacacagagcccctccttcctgagtgccagcgtgggggatagggtgacgatcacatgcaaggcttctcaatcagtggactacagcggagactcctacttgaactggtatcagcaaaagccgggtaaggcccccaagctcctgatctatgatgcgagcaatttggtctccggcgtcccctctaggtttagcggctcaggaagcggcaccgaatttaccctcaccatcagttccctgcagcccgaggattttgcaacgtactattgccagcagagcactgagaacccttggaccttcgggggcggaactaagttagagatcaaaggcggtggagggagcggcggaggcgggtctgggggaggcggtagccaagtgcagcttgtgcaaagtggtgcggaagtcaagaaaccaggctcctcagtcaaagtgtcgtgtaaagcctccgggtacgcctttagctcttactggatgaattgggtcaggcaggcccctggccaaggcctcgaatggatgggtcagatttggccaggcgatagcgacactaattatgctcaaaaattccaggggagagtaaccataacagctgacgaatccacttccacagcctatatggaattaagttcacttcgttctgaggatactgcagtgtattattgcgcccgaagggagaccaccacagtgggccggtactattacgccatggactactggggacagggaaccactgttacagtaagttctactaccacaccagctcctcggcctcctacaccggcccccacaatcgcatcacagcccctcagcctgcggcccgaggcgtgccggccagcagctggcggagccgttcacacccgagggctggacttcgcctgcgacatttatatatgggcaccactcgcagggacctgtggggtcttgctcctgtcccttgtgatcacgctttattgtaaaagaggtagaaaaaagttgttatacattttcaagcagcctttcatgaggcccgtgcagacaacgcaagaagaggatggttgctcttgtagattcccagaggaggaggagggaggctgtgagctccgggtgaagtttagtcgatcggctgatgctcccgcctacaagcagggacagaaccagctttacaatgaactgaacctaggtcgcagagaggagtacgatgttctggacaagagacgcggacgtgacccagaaatgggcggaaaaccgcgccggaaaaacccacaggaaggcctgtataacgaactacagaaagacaagatggctgaggcctacagcgagataggaatgaaaggcgaaaggcgccgagggaagggacacgatggcctgtaccaggggctgtcaaccgcgaccaaggacacttacgacgcactgcatatgcaggctctaccacctcgctgataggatccctggtactgcatgcacgcaatctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgatatccacctgcgccgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgcggccgcgaattcggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 559 - protein, 709 aa, synthetic construct
  source 1..709: note = Anti-hCD8#1 (MX2862) [scFab_ahCD8_OKT8xscFab_ahCD8_OKT8 hIgG1LALAPA-C], protein sequence 1
DIVMTQSPASLAVSLGQRATISCRASESVDSYDNSLMHWYQQKPGQPPKVLIYLASNLESGVPARFSGSGSRTDFTLTIDPVEADDAATYYCQQNNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLLESGPELLKPGASVKMSCKASGYTFTDYNMHWVKQSHGKSLEWIGYIYPYTGGTGYNQKFKNKATLTVDSSSSTAYMELRSLTSEDSAVYYCARNFRYTYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 560 - protein, 707 aa, synthetic construct
  source 1..707: note = Anti-hCD8#1 (MX2862) [scFab_ahCD8_OKT8xscFab_ahCD8_OKT8 hIgG1LALAPA-C], protein sequence 2
DIVMTQSPASLAVSLGQRATISCRASESVDSYDNSLMHWYQQKPGQPPKVLIYLASNLESGVPARFSGSGSRTDFTLTIDPVEADDAATYYCQQNNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLLESGPELLKPGASVKMSCKASGYTFTDYNMHWVKQSHGKSLEWIGYIYPYTGGTGYNQKFKNKATLTVDSSSSTAYMELRSLTSEDSAVYYCARNFRYTYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 561 - DNA, 2127 bp, synthetic construct
  source 1..2127: mol_type = other DNA; note = Anti-hCD8#1 (MX2862) [scFab_ahCD8_OKT8xscFab_ahCD8_OKT8 hIgG1LALAPA-C], DNA sequence 1
gatatcgtaatgacccaaagccctgcctcattagccgtgtccctcgggcagcgggccacgatctcatgcagggcaagcgagtctgtggatagttacgacaacagtctgatgcactggtaccaacagaagcccggccagccgcccaaagtcctgatttatcttgcttccaacctcgaaagcggagttccagcaagattctccggttctggatcgcgcaccgattttactctgacaatagacccagtggaggcggacgacgccgctacatactattgtcagcagaataatgaagatccttatactttcgggggcggcaccaaattggagattaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaggtccagcttctggagagcgggcctgaactcttaaagcccggagcttctgtgaagatgtcctgcaaagctagcgggtataccttcacagattataacatgcactgggtcaagcagagccatggcaaatctctggaatggattggttacatctacccatacacaggtggcaccgggtataatcaaaagtttaaaaacaaggccactctcacagtggactcttcatccagtactgcctacatggagttgcggagcctgacgtcagaggattcggcagtgtactattgtgcgaggaatttcagatacacctactggtattttgacgtttggggccagggaacaaccgtaactgtgtccagtgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggatgt

SEQ ID NO: 562 - DNA, 2121 bp, synthetic construct
  source 1..2121: mol_type = other DNA; note = Anti-hCD8#1 (MX2862) [scFab_ahCD8_OKT8xscFab_ahCD8_OKT8 hIgG1LALAPA-C], DNA sequence 2
gatatcgtaatgacccaaagccctgcctcattagccgtgtccctcgggcagcgggccacgatctcatgcagggcaagcgagtctgtggatagttacgacaacagtctgatgcactggtaccaacagaagcccggccagccgcccaaagtcctgatttatcttgcttccaacctcgaaagcggagttccagcaagattctccggttctggatcgcgcaccgattttactctgacaatagacccagtggaggcggacgacgccgctacatactattgtcagcagaataatgaagatccttatactttcgggggcggcaccaaattggagattaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaggtccagcttctggagagcgggcctgaactcttaaagcccggagcttctgtgaagatgtcctgcaaagctagcgggtataccttcacagattataacatgcactgggtcaagcagagccatggcaaatctctggaatggattggttacatctacccatacacaggtggcaccgggtataatcaaaagtttaaaaacaaggccactctcacagtggactcttcatccagtactgcctacatggagttgcggagcctgacgtcagaggattcggcagtgtactattgtgcgaggaatttcagatacacctactggtattttgacgtttggggccagggaacaaccgtaactgtgtccagtgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 563 - protein, 703 aa, synthetic construct
  source 1..703: note = Anti-hCD8#2 (MX2863) [scFab_ahCD8_ZED8xscFab_ahCD8_ZED8 hIgG1LALAPA-C], protein sequence 1
DVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 564 - protein, 701 aa, synthetic construct
  source 1..701: note = Anti-hCD8#2 (MX2863) [scFab_ahCD8_ZED8xscFab_ahCD8_ZED8 hIgG1LALAPA-C], protein sequence 2
DVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 565 - DNA, 2109 bp, synthetic construct
  source 1..2109: mol_type = other DNA; note = Anti-hCD8#2 (MX2863) [scFab_ahCD8_ZED8xscFab_ahCD8_ZED8 hIgG1LALAPA-C], DNA sequence 1
gacgtccagattacccaatcaccttccagcctttcggcctccgtgggagatagggtaacaatcacctgcagaacgagtcggagcatttctcaatacctggcatggtaccaggaaaagccaggaaagacaaataaattattgatttacagtggctcaactctccagtctggcatacccagccgcttttctgggagcgggtccggtactgatttcacactgaccatctcctcactgcagcccgaagacttcgctacctattattgtcagcaggtgaacgagtttcctccaactttcggccagggtacaaaagttgagatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctccgaagttcaacttgttcaaagtggcgccgaggtgaaaaagccaggggcatctgtaaaggtcagctgcaaagcgtccggcttcaatatcaaggacacatacattcattgggtgagacaggctcctgggcaggggttggagtggatcggccgcatagatcccgccaacgataacacgctgtacgcatcaaaatttcagggtagggccaccattaccgctgacacttcaacatccactgcctacctggaactgagtagcttacgatctgaggacacagctgtgtactattgtggacggggatatggatattatgtcttcgatcactggggccagggtaccctcgtgaccgtgtccagcgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggatgt

SEQ ID NO: 566 - DNA, 2103 bp, synthetic construct
  source 1..2103: mol_type = other DNA; note = Anti-hCD8#2 (MX2863) [scFab_ahCD8_ZED8xscFab_ahCD8_ZED8 hIgG1LALAPA-C], DNA sequence 2
gacgtccagattacccaatcaccttccagcctttcggcctccgtgggagatagggtaacaatcacctgcagaacgagtcggagcatttctcaatacctggcatggtaccaggaaaagccaggaaagacaaataaattattgatttacagtggctcaactctccagtctggcatacccagccgcttttctgggagcgggtccggtactgatttcacactgaccatctcctcactgcagcccgaagacttcgctacctattattgtcagcaggtgaacgagtttcctccaactttcggccagggtacaaaagttgagatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctccgaagttcaacttgttcaaagtggcgccgaggtgaaaaagccaggggcatctgtaaaggtcagctgcaaagcgtccggcttcaatatcaaggacacatacattcattgggtgagacaggctcctgggcaggggttggagtggatcggccgcatagatcccgccaacgataacacgctgtacgcatcaaaatttcagggtagggccaccattaccgctgacacttcaacatccactgcctacctggaactgagtagcttacgatctgaggacacagctgtgtactattgtggacggggatatggatattatgtcttcgatcactggggccagggtaccctcgtgaccgtgtccagcgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga

SEQ ID NO: 567 - protein, 713 aa, synthetic construct
  source 1..713: note = Anti-hCD2 (MX2864) [scFab_ahCD2_sipxscFab_ahCD2_sip hIgG1LALAPA-C], protein sequence 1
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARGRTEYIVVAEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGC

SEQ ID NO: 568 - protein, 711 aa, synthetic construct
  source 1..711: note = Anti-hCD2 (MX2864) [scFab_ahCD2_sipxscFab_ahCD2_sip hIgG1LALAPA-C], protein sequence 2
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARGRTEYIVVAEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 569 - DNA, 2139 bp, synthetic construct
  source 1..2139: mol_type = other DNA; note = Anti-hCD2 (MX2864) [scFab_ahCD2_sipxscFab_ahCD2_sip hIgG1LALAPA-C], DNA sequence 1
gatgtggtcatgacgcagtctccacctagcctgctggtgacacttggacaacccgccagcatttcctgtcggagctcacagtctttactgcactcctccggcaacacatatttgaattggcttctccagaggcccggacagtcacctcagccgttgatatacctcgtgagcaaactggaatctggtgttccagacagatttagtggaagtgggtcggggaccgacttcaccctgaaaatctccggggtcgaggctgaggatgtgggcgtatactactgcatgcaattcactcattatccctatacctttggtcagggcactaagctagaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaagtgcaactggtgcagagtggcgcggaggttaaaaagcctggcgccagcgtcaaggtttcatgtaaggcttccggatacactttcactgggtactatatgcactgggtccgccaggccccaggacagggtcttgagtggatggggagaattaaccccaatagtggggggaccaactatgcccagaaattccagggtcgggtgacaatgacaagggatacatctataagcaccgcctatatggaactgtcacgactcaggtccgatgacaccgcagtgtattactgcgctagaggccgtaccgaatacatcgtggtggcagagggctttgactactggggccagggaacgttggtaactgtcagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgatgcacgaggccctgcacaaccactacacccagaagtccctgagcctgtctcctggaggatgt

SEQ ID NO: 570 - DNA, 2133 bp, synthetic construct
  source 1..2133: mol_type = other DNA; note = Anti-hCD2 (MX2864) [scFab_ahCD2_sipxscFab_ahCD2_sip hIgG1LALAPA-C], DNA sequence 2
gatgtggtcatgacgcagtctccacctagcctgctggtgacacttggacaacccgccagcatttcctgtcggagctcacagtctttactgcactcctccggcaacacatatttgaattggcttctccagaggcccggacagtcacctcagccgttgatatacctcgtgagcaaactggaatctggtgttccagacagatttagtggaagtgggtcggggaccgacttcaccctgaaaatctccggggtcgaggctgaggatgtgggcgtatactactgcatgcaattcactcattatccctatacctttggtcagggcactaagctagaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggaggcggttccggcggaggtggtagtggtggtgggggctcaggtggaggggggagtgggggaggcggatctggcggtggcggatctggaggtggaggatcaggtggaggaggctcccaagtgcaactggtgcagagtggcgcggaggttaaaaagcctggcgccagcgtcaaggtttcatgtaaggcttccggatacactttcactgggtactatatgcactgggtccgccaggccccaggacagggtcttgagtggatggggagaattaaccccaatagtggggggaccaactatgcccagaaattccagggtcgggtgacaatgacaagggatacatctataagcaccgcctatatggaactgtcacgactcaggtccgatgacaccgcagtgtattactgcgctagaggccgtaccgaatacatcgtggtggcagagggctttgactactggggccagggaacgttggtaactgtcagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttgtacccttcctccaagccgggatgagctgaccaagaaccaggtgtccctgagctgtgccgtgaagggcttctaccccagcgacattgccgtggaatgggagagcaatggccagcctgagaacaactacaagacaacccctcctgtgctggacagcgacggctcattcttcctggtgagcaagctgacagtcgacaagtccagatggcagcagggcaacgtgttcagttgcagcgtgatgcacgaggccctgcacaaccactacacccagaagtccctgagcctgtctcctgga